(19) United States
(12) Patent Application Publication
(10) Pub. No.: US 2025/0090687 A1
(43) Pub. Date: Mar. 20, 2025

(54) MITOCHONDRIAL BASE EDITORS AND METHODS FOR EDITING MITOCHONDRIAL DNA

(71) Applicants: The Broad Institute, Inc., Cambridge, MA (US); President and Fellows of Harvard College, Cambridge, MA (US)

(72) Inventors: David R. Liu, Cambridge, MA (US); Julian Wills, Cambridge, MA (US)

(73) Assignees: The Broad Institute, Inc., Cambridge, MA (US); President and Fellows of Harvard College, Cambridge, MA (US)

(21) Appl. No.: 18/957,358

(22) Filed: Nov. 22, 2024

Related U.S. Application Data
(63) Continuation of application No. PCT/US2023/067558, filed on May 26, 2023.
(60) Provisional application No. 63/388,815, filed on Jul. 13, 2022.
(60) Provisional application No. 63/346,639, filed on May 27, 2022.

(51) Int. Cl. A61K 48/00; C12N 9/22; C12N 9/80; C12N 15/11; C12N 15/86

(52) U.S. Cl. CPC A61K 48/005; C12N 9/22; C12N 9/80; C12N 15/111; C12N 15/86; C07K 2319/09; C07K 2319/095; C07K 2319/81; C12N 2310/20; C12N 2750/14143; C12Y 305/04005

(57) ABSTRACT

The present disclosure provides zinc finger domain-containing proteins comprising optimized α-, β-, and linker motifs, and fusion proteins comprising said zinc finger domain-containing proteins fused to an effector domain. The present disclosure also provides double-stranded DNA deaminase A (DddA) variants and fusion proteins comprising said DddA variants fused to a programmable DNA binding protein (e.g., any of the zinc finger domain-containing proteins disclosed herein, a TALE protein, or a CRISPR/Cas9 protein). Methods for editing DNA (including genomic DNA and mitochondrial DNA) using the fusion proteins described herein are also provided by the present disclosure. The present disclosure further provides polynucleotides, vectors, cells, kits, and pharmaceutical compositions comprising the zinc finger domain-containing proteins, DddA variants, and fusion proteins described herein.

### RELATED APPLICATIONS

This application claims priority under 35 U.S.C. 119(e) to U.S. Provisional Application Ser. No. 63/346,639, filed May 27, 2022, and to U.S. Provisional Application Ser. No. 63/388,815, filed Jul. 13, 2022, the contents of each of which are incorporated by reference herein.

### GOVERNMENT SUPPORT

This invention was made with government support under Grant Nos. RM1HG009490, R01EB027793, R01EB031172, R35GM118062, U01A1142756, and T32GM095450 awarded by the National Institutes of Health. The government has certain rights in the invention.

### BACKGROUND OF THE INVENTION

Inherited or acquired mutations in mitochondrial DNA (mtDNA) can profoundly impact cell physiology and are associated with a spectrum of human diseases, ranging from rare inborn errors of metabolism, certain cancers, age-associated neurodegeneration, and even the aging process itself. Tools for introducing specific modifications to mtDNA are needed both for modeling diseases and for their therapeutic potential. The development of such tools, however, has been constrained in part by the challenge of transporting RNAs into mitochondria, including guide RNAs required to facilitate nucleic acid modification and/or editing using CRISPR-associated proteins.

Each mammalian cell contains hundreds to thousands of copies of circular mtDNA. Homoplasmy refers to a state in which all mtDNA molecules are identical, while heteroplasmy refers to a state in which a cell contains a mixture of wild-type and mutant mtDNA. Current approaches to engineering and/or altering mtDNA rely on RNA-free DNA-binding proteins, such as transcription activator-like effector nucleases (mitoTALENs) and zinc finger nucleases fused to mitochondrial targeting sequences (mitoZFNs), to induce double-strand breaks (DSBs). Upon cleavage, the linearized mtDNA is rapidly degraded, resulting in heteroplasmic shifts to favor uncut mtDNA genomes. As a candidate therapy however, this approach cannot be applied to homoplasmic mtDNA mutations since destroying all mtDNA copies is presumed to be harmful. In addition, using DSBs to eliminate heteroplasmic mtDNA mutations, which tend to be functionally recessive, implicitly requires the edited cell to restore its wild-type mtDNA copy number. During this transient period of mtDNA repopulation, the loss of mtDNA copies could cause cellular toxicity resulting in deleterious effects (e.g., apoptosis).

A favorable alternative to targeted destruction of DNA through DSBs is precision genome editing. The ability to precisely install or correct pathogenic mutations, rather than destroy targeted mtDNA, could accelerate the ability to model mtDNA diseases in cells and animal models, and in principle could also enable therapeutic approaches that correct pathogenic mtDNA and genomic DNA mutations.

Therefore, the development of programmable base editors that are capable of introducing a nucleotide change and/or that could alter or modify the nucleotide sequence at a target site with high specificity and efficiency within DNA, including genomic DNA and mtDNA, would substantially expand the scope and therapeutic potential of genome editing technologies.

### SUMMARY OF THE INVENTION

The present disclosure is based on the development of engineered zinc finger domain-containing proteins, engineered double-stranded DNA deaminase A (DddA variants), and fusion proteins comprising engineered zinc finger domain-containing proteins and/or engineered DddA variants that display increased on-target base editing activity and/or decreased off-target base editing activity, including when acting on mtDNA. Thus, in one aspect, the present disclosure provides engineered zinc finger domain-containing proteins comprising (i) one or more linker motifs, wherein each linker motif independently comprises the amino acid sequence of any one of SEQ ID NOs: 1-24; (ii) one or more α-motifs, wherein each α-motif independently comprises the amino acid sequence of any one of SEQ ID NOs: 25-42 and 346; and (iii) one or more β-motifs, wherein each β-motif independently comprises the amino acid sequence of any one of SEQ ID NOs: 43-138 and 336-345, or an amino acid sequence that is at least 90%, at least 95%, or at least 99% identical to the amino acid sequence of any one of SEQ ID NOs: 43-138 and 336-345. In some embodiments, a zinc finger domain-containing protein comprises the structure [first β-motif]-[first DNA recognition motif]-[first α-motif]-[first linker motif]-[second β-motif]-[second DNA recognition motif]-[second α-motif]-[second linker motif]-[third β-motif]-[third DNA recognition motif]-[third α-motif]. In certain embodiments, each of the first, second, and third β-motifs comprise the same amino acid sequence, each of the first, second, and third α-motifs comprise the same amino acid sequence, and/or each of the first and second linker motifs comprise the same amino acid sequence. In some embodiments, a zinc finger domain-containing protein comprises the structure [first β-motif]-[first DNA recognition motif]-[first α-motif]-[first linker motif]-[second β-motif]-[second DNA recognition motif]-[second α-motif]-[second linker motif]-[third β-motif]-[third DNA recognition motif]-[third α-motif]-[third linker motif]-[fourth β-motif]-[fourth DNA recognition motif]-[fourth α-motif]. In certain embodiments, each of the first, second, third, and fourth β-motifs comprise the same amino acid sequence, each of the first, second, third, and fourth α-motifs comprise the same amino acid sequence, and/or each of the first, second, and third linker motifs comprise the same amino acid sequence. In some embodiments, a zinc finger domain-containing protein comprises the structure [first β-motif]-[first DNA recognition motif]-[first α-motif]-[first linker motif]-[second β-motif]-[second DNA recognition motif]-[second α-motif]-[second linker motif]-[third β-motif]-[third DNA recognition motif]-[third α-motif]-[third linker motif]-[fourth β-motif]-[fourth DNA recognition motif]-[fourth α-motif]-[fourth linker motif]-[fifth β-motif]-[fifth DNA recognition motif]-[fifth α-motif]. In certain embodiments, each of the first, second, third, fourth, and fifth β-motifs comprise the same amino acid sequence, each of the first, second, third, fourth, and fifth α-motifs comprise the same amino acid sequence, and/or each of the first, second, third, and fourth linker motifs comprise the same amino acid sequence. In some embodiments, a zinc finger domain-containing protein comprises the structure [first β-motif]-[first DNA recognition motif]-[first α-motif]-[first linker motif]-[second β-motif]-[second DNA recognition motif]-[second α-motif]-[second linker motif]-[third β-motif]-[third DNA recognition motif]-[third α-motif]-[third linker motif]-[fourth β-motif]-[fourth DNA recognition motif]-[fourth α-motif]-[fourth linker motif]-[fifth β-motif]-[fifth DNA recognition motif]-[fifth α-motif]-[fifth linker motif]-[sixth β-motif]-[sixth DNA recognition motif]-[sixth α-motif]. In certain embodiments, each of the first, second, third, fourth, fifth, and sixth β-motifs comprise the same amino acid sequence, each of the first, second, third, fourth, fifth, and sixth α-motifs comprise the same amino acid sequence, and each of the first, second, third, fourth, and fifth linker motifs comprise the same amino acid sequence. In some embodiments, any of the zinc finger domain-containing proteins provided herein may comprise an N-terminal cap (e.g., the amino acid sequence MAERP). In some embodiments, any of the zinc finger domain-containing proteins provided herein may comprise a C-terminal cap (e.g., the amino acid sequence HTKIHLR).

Each of the linker, alpha, and beta motifs may comprise or consist of any of the various amino acid sequences provided herein, in any combination with one another. In certain preferred embodiments, the present disclosure provides zinc finger domain-containing proteins that comprise multiple instances of the same linker sequence, the same beta motif sequence, and the same alpha motif sequence, including embodiments in which the zinc finger protein comprises the same sequence for all instances of the linker motif within the protein, the same sequence for all instances of the beta motif within the protein, and the same sequence for all instances of the alpha motif within the protein.

In some embodiments, a zinc finger domain-containing protein comprises one or more linker motifs comprising the amino acid sequence of any one of TGEKP (SEQ ID NO: 1), SGEKP (SEQ ID NO: 13), SGERP (SEQ ID NO: 14), and SGDKP (SEQ ID NO: 17). In certain embodiments, all of the linker motifs present in a zinc finger domain-containing protein each comprise the same amino acid sequence selected from the group consisting of TGEKP (SEQ ID NO: 1), SGEKP (SEQ ID NO: 13), SGERP (SEQ ID NO: 14), and SGDKP (SEQ ID NO: 17).

In some embodiments, a zinc finger domain-containing protein comprises one or more α-motifs comprising the amino acid sequence of any one of HMRTH (SEQ ID NO: 33), HMKIH (SEQ ID NO: 34), HMKVH (SEQ ID NO: 35), HMKTH (SEQ ID NO: 36), and HIRTH (SEQ ID NO: 346). In certain embodiments, all of the α-motifs present in a zinc finger domain-containing protein each comprise the same amino acid sequence selected from the group consisting of HMRTH (SEQ ID NO: 33), HMKIH (SEQ ID NO: 34), HMKVH (SEQ ID NO: 35), HMKTH (SEQ ID NO: 36), and HIRTH (SEQ ID NO: 346).

In some embodiments, a zinc finger domain-containing protein comprises one or more β-motifs comprising the amino acid sequence of any one of YKCNECGKAFN (SEQ ID NO: 51), YKCNECGKSFN (SEQ ID NO: 54), YKCSECGKAFN (SEQ ID NO: 57), YKCEECGKAFN (SEQ ID NO: 63), FKCNECGKAFN (SEQ ID NO: 99), FKCNECGKSFN (SEQ ID NO: 102), FKCSECGKAFN (SEQ ID NO: 105), FKCEECGKAFS (SEQ ID NO: 109), FKCEECGKAFN (SEQ ID NO: 111), FKCEECGKSFN (SEQ ID NO: 114), YACPECGKSFS (SEQ ID NO: 337), and FACDICGRKFA (SEQ ID NO: 345). In certain embodiments, all of the β-motifs present in a zinc finger domain-containing protein each comprise the same amino acid sequence selected from the group consisting of YKCNECGKAFN (SEQ ID NO: 51), YKCNECGKSFN (SEQ ID NO: 54), YKCSECGKAFN (SEQ ID NO: 57), YKCEECGKAFN (SEQ ID NO: 63), FKCNECGKAFN (SEQ ID NO: 99), FKCNECGKSFN (SEQ ID NO: 102), FKCSECGKAFN (SEQ ID NO: 105), FKCEECGKAFS (SEQ ID NO: 109), FKCEECGKAFN (SEQ ID NO: 111), FKCEECGKSFN (SEQ ID NO: 114), YACPECGKSFS (SEQ ID NO: 337), and FACDICGRKFA (SEQ ID NO: 345).

In certain embodiments, the present disclosure provides zinc finger domain-containing proteins in which every β-motif comprises the amino acid sequence FACDICGRKFA (SEQ ID NO: 345), every α-motif comprises the amino acid sequence HIRTH (SEQ ID NO: 346), and every linker motif comprises the amino acid sequence TGEKP (SEQ ID NO: 1). In certain embodiments, every β-motif comprises the amino acid sequence YACPECGKSFS (SEQ ID NO: 337), every α-motif comprises the amino acid sequence HIRTH (SEQ ID NO: 346), and every linker motif comprises the amino acid sequence TGEKP (SEQ ID NO: 1). In certain embodiments, every β-motif comprises the amino acid sequence FKCEECGKAFN (SEQ ID NO: 111), every α-motif comprises the amino acid sequence HIRTH (SEQ ID NO: 346), and every linker motif comprises the amino acid sequence TGEKP (SEQ ID NO: 1). In certain embodiments, every β-motif comprises the amino acid sequence YKCEECGKAFN (SEQ ID NO: 63), every α-motif comprises the amino acid sequence HIRTH (SEQ ID NO: 346), and every linker motif comprises the amino acid sequence TGEKP (SEQ ID NO: 1).

In another aspect, the present disclosure provides fusion proteins comprising any of the zinc finger domain-containing proteins disclosed herein, and an effector protein. In some embodiments, the effector protein comprises nuclease activity, nickase activity, recombinase activity, deaminase activity, methyltransferase activity, methylase activity, acetylase activity, acetyltransferase activity, transcriptional activation activity, transcriptional repression activity, or polymerase activity. In some embodiments, the effector protein is a nucleic acid editing protein, such as a deaminase (e.g., an adenosine deaminase or a cytidine deaminase). In certain embodiments, the effector protein comprises a double-stranded DNA cytidine deaminase (DddA) domain. The fusion proteins provided herein may, in some embodiments, comprise one or more additional domains such as one or more mitochondrial targeting sequences, one or more nuclear export sequences (e.g., the NES of mitogen-activated protein kinase kinase (MAPKK)), one or more nuclear localization sequences, and/or one or more UGI domains. In some embodiments, the zinc finger domain-containing protein and the effector protein are joined by a linker (e.g., a glycine and serine-rich amino acid linker, optionally wherein the linker is about 13 amino acids in length). In certain embodiments, the fusion proteins comprise the structure NH₂-[MTS]-[FLAG tag]-[NES]-[NES]-[first zinc finger domain]-[second zinc finger domain]-[third zinc finger domain]-[optional fourth zinc finger domain]-[optional fifth zinc finger domain]-[optional sixth zinc finger domain]-[linker]-[split DddA]-[UGI]-COOH or NH₂-[MTS]-[FLAG tag]-[NES]-[NES]-[split DddA]-[linker]-[first zinc finger domain]-[second zinc finger domain]-[third zinc finger domain]-[optional fourth zinc finger domain]-[optional fifth zinc finger domain]-[optional sixth zinc finger domain]-[UGI]-COOH.

In another aspect, the present disclosure provides double-stranded DNA cytidine deaminase (DddA) variants comprising a first fragment comprising an amino acid sequence that is at least 80%, at least 85%, at least 90%, at least 95%, or at least 99% identical to the amino acid sequence of SEQ ID NO: 139, and a second fragment comprising an amino acid sequence that is at least 80%, at least 85%, at least 90%, at least 95%, or at least 99% identical to the amino acid sequence of SEQ ID NO: 283, wherein the first fragment comprises one or more amino acid substitutions, truncations, or extensions relative to the amino acid sequence of SEQ ID NO: 139, and/or wherein the second fragment comprises one or more amino acid substitutions, truncations, or extensions relative to the amino acid sequence of SEQ ID NO: 283. The DddA variants provided by the present disclosure may comprise one or more modifications relative to a wild type DddA sequence including, but not limited to, one or more point mutations, and N- and/or C-terminal amino acid truncations and/or extensions.

In some embodiments, the first fragment of a DddA variant comprises one or more amino acid substitutions relative to the amino acid sequence of SEQ ID NO: 139. In some embodiments, the first fragment of a DddA variant comprises an amino acid sequence of any one of SEQ ID NOs: 140-252, or an amino acid sequence at least 80%, at least 85%, at least 90%, at least 95%, or at least 99% identical to the amino acid sequence of any one of SEQ ID NOs: 140-252. In some embodiments, the first fragment of a DddA variant comprises an amino acid substitution at position N18. In certain embodiments, the amino acid substitution is an N18K substitution. In some embodiments, the first fragment of a DddA variant comprises an amino acid substitution at position P25. In certain embodiments, the amino acid substitution is a P25K substitution. In certain embodiments, the amino acid substitution is a P25A substitution.

In some embodiments, the first fragment of a DddA variant comprises an N-terminal amino acid truncation. In some embodiments, the first fragment of a DddA variant comprises an N-terminal amino acid truncation of 1-15 amino acids in length. In certain embodiments, the first fragment of a DddA variant comprises the amino acid sequence of any one of SEQ ID NOs: 253-267.

In some embodiments, the first fragment of a DddA variant comprises a C-terminal amino acid truncation. In some embodiments, the first fragment of a DddA variant comprises a C-terminal amino acid truncation of 1-15 amino acids in length. In certain embodiments, the first fragment of a DddA variant comprises the amino acid sequence of any one of SEQ ID NOs: 268-282.

In some embodiments, the second fragment of a DddA variant comprises a C-terminal amino acid truncation. In some embodiments, the second fragment of a DddA variant comprises a C-terminal amino acid truncation of 1-10 amino acids in length. In certain embodiments, the second fragment of a DddA variant comprises a C-terminal amino acid truncation of 3 amino acids in length. In certain embodiments, the first fragment of a DddA variant comprises the amino acid sequence of any one of SEQ ID NOs: 284-293.

In some embodiments, the second fragment of a DddA variant comprises a C-terminal amino acid extension. In some embodiments, the second fragment of a DddA variant comprises a C-terminal amino acid extension of 1-15 amino acids in length. In certain embodiments, the first fragment of a DddA variant comprises the amino acid sequence of any one of SEQ ID NOs: 294-308.

In some embodiments, a DddA variant further comprises a sequence of charged amino acid residues (e.g., of the amino acid sequence of any one of SEQ ID NOs: 309-334) to weaken the binding affinity of the first fragment and the second fragment of the DddA variant to one another.

In some embodiments, a DddA variant further comprises a catalytically dead second DddA fragment fused to the first DddA fragment. In some embodiments, the catalytically dead second DddA fragment comprises the amino acid sequence of SEQ ID NO: 335, or an amino acid sequence that is at least 80%, at least 85%, at least 90%, at least 95%, or at least 99% identical to the amino acid sequence of SEQ ID NO: 335.

In certain embodiments, the present disclosure provides a DddA variant comprising a first fragment that comprises amino acid substitutions at positions N18 (e.g., an N18K substitution) and P25 (e.g., a P25A or P25K substitution), and a second fragment that comprises a C-terminal amino acid truncation of 3 amino acids in length.

In another aspect, the present disclosure provides fusion proteins comprising a programmable DNA binding protein and a first or second fragment of any of the DddA variants provided herein. In some embodiments, the programmable DNA binding protein is a nucleic acid-programmable DNA binding protein (napDNAbp), e.g., a Cas9 protein (including Cas9 nickases and nuclease-inactive Cas9 proteins). In some embodiments, the napDNAbp is selected from the group consisting of Cas9, Cas12e, Cas12d, Cas12a, Cas12b1, Cas13a, Cas12c, and Argonaute, and optionally has a nickase activity. In some embodiments, the programmable DNA binding protein is a zinc finger protein, such as any of the zinc finger domain-containing proteins disclosed herein. In some embodiments, the programmable DNA binding protein is a TALE protein. The fusion proteins provided herein may, in certain embodiments, comprise one or more additional domains such as one or more mitochondrial targeting sequences, one or more nuclear export sequences (e.g., the NES of mitogen-activated protein kinase kinase (MAPKK)), one or more nuclear localization sequences, and/or one or more UGI domains. In some embodiments, the pDNAbp and the first or second fragment of the DddA variant are joined by a linker (e.g., a glycine and serine-rich amino acid linker, optionally wherein the linker is about 13 amino acids in length). In certain embodiments, the fusion proteins comprise the structure NH₂-[MTS]-[FLAG tag]-[NES]-[NES]-[first zinc finger domain]-[second zinc finger domain]-[third zinc finger domain]-[optional fourth zinc finger domain]-[optional fifth zinc finger domain]-[optional sixth zinc finger domain]-[linker]-[split DddA]-[UGI]-COOH or NH₂-[MTS]-[FLAG tag]-[NES]-[NES]-[split DddA]-[linker]-[first zinc finger domain]-[second zinc finger domain]-[third zinc finger domain]-[optional fourth zinc finger domain]-[optional fifth zinc finger domain]-[optional sixth zinc finger domain]-[UGI]-COOH.

In another aspect, the present disclosure provides fusion proteins comprising any of the zinc finger domain-containing proteins provided herein and the first or second fragment of any of the DddA variants provided herein.

In another aspect, the present disclosure provides methods for editing a target nucleic acid molecule comprising contacting the target nucleic acid molecule with any of the fusion proteins disclosed herein. The target nucleic acid molecule may comprise, for example, nuclear DNA or mitochondrial DNA. In some embodiments, the contacting is performed in vitro. In some embodiments, the contacting is performed in vivo (e.g., in a subject). In some embodiments, the contacting is performed in a subject that has been diagnosed with a disease or disorder. In some embodiments, the target sequence comprises a genomic sequence associated with a disease or disorder. For example, the target sequence may comprise a point mutation associated with a disease or disorder, such as a T→C point mutation associated with a disease or disorder or an A→G point mutation associated with a disease or disorder. In some embodiments, the step of editing the target nucleic acid results in correction of the point mutation. In some embodiments, the target nucleic acid comprises MT-TK, Nd1, HBB, or MT-TL1. In certain embodiments, the fusion protein used in the methods provided herein comprises the architecture of any of the fusion proteins provided in Table 7, Table 8, and Table 31.

In another aspect, the present disclosure provides polynucleotides encoding any of the zinc finger domain-containing proteins, DddA variants, or fusion proteins provided herein. In another aspect, the present disclosure provides vectors comprising any of the polynucleotides provided herein.

In another aspect, the present disclosure provides cells comprising any of the zinc finger domain-containing proteins, DddA variants, fusion proteins, polynucleotides, or vectors provided herein.

In another aspect, the present disclosure provides kits comprising any of the zinc finger domain-containing proteins, DddA variants, fusion proteins, polynucleotides, vectors, or cells provided herein.

In another aspect, the present disclosure provides pharmaceutical compositions comprising any of the zinc finger domain-containing proteins, DddA variants, fusion proteins, polynucleotides, or vectors provided herein, and a pharmaceutically acceptable excipient.

In another aspect, the present disclosure provides AAVs comprising any of the fusion proteins, polynucleotides, or vectors provided herein.

In some embodiments, any of the zinc finger domain-containing proteins, DddA variants, fusion proteins, polynucleotides, vectors, pharmaceutical compositions, and AAVs provided herein may be for use in medicine. In some embodiments, the present disclosure provides for the use of any of the zinc finger domain-containing proteins, DddA variants, fusion proteins, polynucleotides, vectors, pharmaceutical compositions, and AAVs disclosed herein in the manufacture of a medicament for the treatment of a disease or disorder.

It should be appreciated that the foregoing concepts, and additional concepts discussed below, may be arranged in any suitable combination, as the present disclosure is not limited in this respect. Further, other advantages and novel features of the present disclosure will become apparent from the following detailed description of various non-limiting embodiments when considered in conjunction with the accompanying figures.

### BRIEF DESCRIPTION OF THE DRAWINGS

The following drawings form part of the present specification and are included to further demonstrate certain aspects of the present disclosure, which can be better understood by reference to one or more of these drawings in combination with the detailed description of specific embodiments presented herein.

FIGS. 1A-1E: Architectural improvements increase zinc finger double-stranded DNA deaminase cytosine base editor (ZF-DdCBE) editing activity. A schematic of evolution of DddA via PACE is shown in FIG. 1C.

FIG. 2: Schematic of C-terminal ZF-DdCBE architecture.

FIG. 3: Schematic of N- or C-terminal ZF-DdCBE architecture.

FIGS. 4A-4E: Canonical zinc finger scaffolds. Typical consensus sequences for a 3ZF array (FIG. 4A), a 4ZF array (FIG. 4B), a 5ZF array (FIG. 4C), and a 6ZF array (FIG. 4D) are shown. FIG. 4E provides exemplary sequences of the zinc finger proteins shown in FIGS. 4A-4D comprising different variable DNA-binding residues.

FIGS. 5A-5C: Testing of permutations of β-motif, α-motif, and linker motif combinations to find improved ZF scaffolds. X1 represents a single 1ZF protein

FIGS. 6A-6D: Improvements of variant X1 hold across different ZF array lengths and different sites.

FIG. 7: Schematic representing workflow for finding further improvements for optimized ZF scaffolds.

FIG. 8: Data from searching the human proteome for ZF sequences.

FIGS. 9A-9B: Identification of linker motif consensus sequences.

FIG. 10: Percent C to T editing efficiency for various diverse linker motifs tested to improve ZF activity.

FIG. 11: Percent C to T editing for top linker motifs.

FIGS. 12A-12B: Identification of α-motif consensus sequences.

FIG. 13: Percent C to T editing efficiency for various diverse α-motifs tested to improve ZF activity.

FIG. 14: Percent C to T editing for top α-motifs.

FIGS. 15A-15B: Identification of β-motif consensus sequences.

FIGS. 16A-16D: Percent C to T editing efficiency for various diverse β-motifs tested to improve ZF activity.

FIG. 17: Percent C to T editing for top β-motifs.

FIG. 18: Schematic showing workflow for combining improvements in β-motifs, α-motifs, and linker motifs to produce optimized ZF scaffolds.

FIG. 19: TALE-DdCBEs exhibit minimal off-target editing.

FIG. 20: Amplicon-wide sequencing reveals off-target editing by ZF-DdCBEs.

FIG. 21: Average amplicon-wide percent C to T or G to A editing shows that off-target editing is caused by DddA.

FIG. 22: Architectural differences underlie the discrepancy in DddA off-target editing.

FIGS. 23A-23C: Off-target editing depends on the interaction strength between split deaminase halves.

FIG. 24: Schematic showing tuning of the interaction strength between split deaminase halves.

FIG. 25: Structure of a split double-stranded DNA deaminase, split at amino acid position G1397. Fragments G1397N and G1397C are shown.

FIG. 26: Structures of truncation options for split DddA.

FIG. 27: Percent on-target activity for various N-terminal truncations of DddA-C and C-terminal truncations of DddA-N.

FIG. 28: Percent off-target activity for various N-terminal truncations of DddA-C and C-terminal truncations of DddA-N.

FIG. 29: Percent on-target activity for various C-terminal truncations of DddA-C and C-terminal truncations of DddA-N.

FIG. 30: Percent off-target activity for various C-terminal truncations of DddA-C and C-terminal truncations of DddA-N.

FIG. 31: Maximizing on-target editing and minimizing off-target editing of DddA.

FIG. 32: Minimizing off-target editing of DddA using truncations.

FIG. 33: Alanine scanning mutagenesis of DddA.

FIG. 34: Lysine scanning mutagenesis of DddA.

FIG. 35: Aspartate scanning mutagenesis of DddA.

FIG. 36: Glutamate scanning mutagenesis of DddA.

FIG. 37: Comparison between positively charged mutations (lysine, arginine, and histidine).

FIGS. 38A-38B: Additive combination of single mutations in DddA (FIG. 38A) and single+double mutations in DddA (FIG. 38B). Percent on-target editing and percent off-target editing are shown.

FIG. 39: Effect of combining mutations and truncations on DddA activity. Percent on-target editing and percent off-target editing are shown.

FIGS. 40A-40B: Capping of DddA with a dead deaminase. A schematic of a capped deaminase is provided (FIG. 40A), and percent on-target editing and average amplicon-wide off-target editing for a dead DddA (dDddA) capped DddA are shown.

FIG. 41: Schematic showing the introduction of charged residues into the flexible linker upstream of DddA.

FIGS. 42A-42C: Percent on-target editing and average-amplicon wide off-target editing for DddA variants incorporating positively charged residues into the upstream flexible linker. Data for incorporation of arginine residues (FIG. 42A), lysine residues (FIG. 42B), and histidine residues (FIG. 42C) are shown.

FIGS. 43A-43B: Percent on-target editing and average-amplicon wide off-target editing for DddA variants incorporating negatively charged residues into the upstream flexible linker. Data for incorporation of aspartate residues (FIG. 43A) and glutamate residues (FIG. 43B) are shown.

FIGS. 44A-44D: Data showing on-target editing and off-target editing demonstrate that orthogonal approaches for improving DddA activity can be combined additively.

FIGS. 45A-45B: Specificity-optimized ZF-DdCBEs reduce off-target editing.

FIGS. 46A-46B: ZF β-motif sequences. FIG. 46A shows the most commonly-used sequences in canonical ZF scaffolds. FIG. 46B shows additional newly defined ZF scaffold sequences.

FIGS. 47A-47D: Example ZF proteins comprising one of the newly defined ZF scaffold sequences from FIG. 46B (X1). A 3ZF array (FIG. 47A), a 4ZF array (FIG. 47B), a 5ZF array (FIG. 47C), and a 6ZF array (FIG. 47D) are shown.

FIGS. 48A-48H: Improved ZF scaffolds show increased editing activity at a panel of different target sites.

FIG. 49: ZF scaffolds for additional β-motif sequences.

FIGS. 50A-50C: Percent on-target editing and average off-target editing for specificity-optimized DddA mutants. In FIGS. 50A and 50B, the three farthest rightmost dots represent canonical DddA scaffolds, and gray dots represent a selection of the most promising DddA mutants based on observed activity.

FIG. 51: Mutations and sequences of improved DddA variants.

FIGS. 52A-52E: Optimizing ZF-DdCBEs increases base editing efficiency in mitochondria. FIG. 52A: Architectures of optimized ZF-DdCBEs showing progression from v1 to v8. The components are a mitochondrial targeting signal, FLAG tag, nuclear export signal(s), ZF array with either canonical ZF scaffold (dark grey) or optimized ZF scaffold (light grey), Gly/Ser-rich flexible linker, split DddA deaminase (with or without activity-enhancing mutations and specificity-enhancing mutations) and UGI. FIGS. 52B-52C: Mitochondrial DNA base editing efficiencies of HEK293T cells treated with (FIG. 52B) six optimized ZF-DdCBE pairs used to establish architectural improvements or (FIG. 52C) seven additional optimized ZF-DdCBE pairs.

FIGS. 52D-52E: Comparison of mitochondrial DNA base editing efficiencies of HEK293T cells treated with either ZFD or optimized ZF-DdCBE pairs at genomic target sites chosen by (FIG. 52D) Lim et al.²⁵, or this study (FIG. 52E). For FIGS. 52B-52E, values and errors reflect the mean±s.d. of n=3 independent biological replicates. The editing efficiencies shown are for the most efficiently edited C•G within the spacing region.

FIGS. 53A-53L: High-specificity ZF-DdCBE variants reduce mitochondrial off-target editing. FIG. 53A: Mitochondrial DNA base editing efficiencies within amplicon ND4 of HEK293T cells treated with ND4-DdCBE. FIG. 53B: Mitochondrial DNA base editing efficiencies within amplicon ATP8 of HEK293T cells treated with v7 ZF-DdCBE pair R8-3i-ATP8+4-3i-ATP8. FIG. 53C: Off-target editing efficiencies within mitochondrial off-target amplicon ND5.1 of HEK293T cells treated with ND4-DdCBE, v7 ZF-DdCBE pair R8-3i-ATP8+4-3i-ATP8, or individual components of the v7 ZF-DdCBE architecture. FIGS. 53D-53L: On-target and average off-target editing efficiencies within amplicon ATP8 of HEK293T cells treated with canonical v7 ZF-DdCBE pair R8-3i-ATP8+4-3i-ATP8 (indicated with an arrow) or variants containing (FIG. 53D) DddA^{N} and DddA^{C} truncations, (FIG. 53E) Ala, (FIG. 53F) Lys, (FIG. 53G) Asp, or (FIG. 53H) Glu point mutations within DddA^{C}, (FIG. 53I) Asp or (FIG. 53J) Glu residues upstream or downstream of DddA^{N} and DddA^{C}, (FIG. 53K) fused catalytically inactivated DddA^{N}, or (FIG. 53L) combinations thereof. High-specificity variants HS1 to HS5 are labeled accordingly. For FIGS. 53A-53B and FIGS. 53D-53L, values reflect the mean of n=3 independent biological replicates. For FIG. 53C, values and errors reflect the mean±s.d. of n=3 independent biological replicates. For FIGS. 53D-53L, the editing efficiencies shown are for the most efficiently edited C•G within the spacing region.

FIGS. 54A-54E: ZF-DdCBEs install pathogenic mutations in cultured cells in vitro. FIG. 54A: The m.8340G>A mutation in human MT-TK disrupts the T-arm of mt-tRNA^{Lys}. FIG. 54B: Mitochondrial DNA base editing efficiencies of HEK293T cells treated with an optimized ZF-DdCBE pair designed to install m.8340G>A. FIG. 54C: The m.7743G>A mutation in mouse Mt-tk disrupts the T-arm of mt-tRNA^{Lys}. FIG. 54D: Mitochondrial DNA base editing efficiencies of C2C12 cells treated with an optimized ZF-DdCBE pair designed to install m.7743G>A. FIG. 54E: Mitochondrial DNA base editing efficiencies of C2C12 cells treated with an optimized ZF-DdCBE pair designed to install m.3177G>A. For FIGS. 54B, 54D, and 54E, values and errors reflect the mean±s.d. of n=3 independent biological replicates. For each site the DNA spacing region, split DddA orientation, ZF array lengths, and ZF-targeted DNA strands (LT=left top; LB=left bottom; RB=right bottom) are shown, and the cytosine with the highest editing efficiency is colored in light gray.

FIGS. 55A-55B: ZF-DdCBEs enable base editing of nuclear DNA. FIG. 55A: Nuclear DNA base editing efficiencies of HEK293T cells treated with five 3ZF+3ZF nuclear-targeted ZF-DdCBE pairs, or ZF-DdCBE variants with extended ZF arrays. The editing efficiencies shown are for the most efficiently edited C•G within the spacing region. FIG. 55B: Nuclear DNA base editing efficiencies of HEK293T-HBB cells treated with an optimized ZF-DdCBE pair designed to correct the HBB-28(A>G) mutation. The DNA spacing region, split DddA orientation, ZF array lengths, and ZF-targeted DNA strands (LT=left top; RB=right bottom) are shown, and the pathogenic cytosine is colored in light gray. For FIGS. 55A-55B, values and errors reflect the mean±s.d. of n=3 independent biological replicates.

FIGS. 56A-56F: In vivo base editing of pathogenic sites in mtDNA. FIG. 56A: Mitochondrial DNA base editing efficiencies installing m.7743G>A of tissue samples from mice treated with buffer, dAAV-Mt-tk, or AAV-Mt-tk. FIG. 56B: Mitochondrial DNA base editing efficiencies of tissue samples from AAV-Mt-tk-treated mice. FIG. 56C: Off-target editing efficiencies within representative mitochondrial off-target amplicon OT8 of tissue samples from mice treated with buffer, dAAV-Mt-tk, or AAV-Mt-tk. FIG. 56D: Mitochondrial DNA base editing efficiencies installing m.3177G>A of tissue samples from mice treated with buffer or AAV-Nd1. FIG. 56E: Mitochondrial DNA base editing efficiencies of tissue samples from AAV-Nd1-treated mice. FIG. 56F: Off-target editing efficiencies within representative mitochondrial off-target amplicon OT7 of tissue samples from mice treated with buffer, or AAV-Nd1. For FIGS. 56A-56B, values and errors reflect the mean±s.d. of n=4, 4 and 7 for mice treated with buffer, AAV-Mt-tk, or dAAV-Mt-tk, respectively. For FIG. 56C, values reflect the mean of n=4, 4 and 7 for mice treated with buffer, AAV-Mt-tk, or dAAV-Mt-tk, respectively. For FIGS. 56D-56E, values and errors reflect the mean±s.d. of n=4 and 7 for mice treated with buffer or AAV-Nd1, respectively. For FIG. 56F, values reflect the mean of n=4 and 7 for mice treated with buffer or AAV-Nd1, respectively.

FIG. 57: All-protein base editor size comparison. The area of each hexagon is proportional to the length of DNA sequence required to encode that protein. The total AAV packaging capacity of ˜4.7 kb is represented proportionally in brown. The total size of DNA encoding a ZF-DdCBE is well below the AAV packaging capacity limit, whereas the total size of DNA encoding a TALE-DdCBE exceeds the packaging limit of a single AAV capsid. The ZF and TALE hexagons each represent a six-zinc finger (6ZF) array and an 18-repeat TALE array, respectively.

FIGS. 58A-58E: ZF-DdCBE architecture optimization. FIG. 58A: Initial mitochondrial ZF-DdCBE pairs used to establish v1 to v5 architectural improvements. For each site the DNA spacing region, split DddA orientation, ZF array lengths, and ZF-targeted DNA strands (LB=left bottom, RT=right top) are shown, and the cytosine with the highest editing efficiency is colored in light gray. ZF-DdCBE naming convention follows A+B where A and B specify the left and right ZF, respectively. Nucleotide numbering starts with the first 5′-nucleotide in the spacing region designated position 1. For R8-ATP8+4-ATP8, nucleotide C5 has the highest editing efficiency. FIGS. 58B-58E: Mitochondrial DNA base editing efficiencies of HEK293T cells treated with four ZF-DdCBE pairs testing the effects of: (FIG. 58B) replacing the two-amino acid linker in architecture v1 with a 7- or 13-amino acid Gly/Ser-rich flexible linker, or a 32-amino acid XTEN linker; (FIG. 58C), inserting a FLAG or HA tag immediately downstream of the MTS in architecture v2; (FIG. 58D), adding an additional NES from HIV-1 Rev (NES1), MAPKK (NES2), or MVM NS2 (NES3) to architecture v3, either downstream of the existing internal NES or at the C-terminus of the protein; or (FIG. 58E), moving the location of UGI within the fusion protein to a position N-terminal of the 5ZF array, appending a second copy of UGI to the C-terminus (2×UGI), or expressing a separate mitochondrially targeted UGI in trans using a self-cleaving P2A peptide (with (P2A UGI only) or without (+P2A UGI) removing the C-terminally fused UGI) compared to architecture v3. Values and errors reflect the mean±s.d. of n=3 independent biological replicates. The editing efficiencies shown are for the most efficiently edited C•G within the spacing region.

FIGS. 59A-59I: ZF array length and positioning influences ZF-DdCBE editing efficiency. FIG. 59A: Truncation of 5ZF arrays to create a set of two 4ZFs and a set of three 3ZFs by removing either one or two individual ZFs, respectively, creates four resulting 4ZF+4ZF combinations and nine 3ZF+3ZF combinations derived from the original 5ZF+5ZF ZF-DdCBE pair. FIGS. 59B-59I: Mitochondrial DNA base editing efficiencies of HEK293T cells treated with truncated v5 ZF-DdCBE pairs derived from (FIG. 59B and FIG. 59F) R8-ATP8+4-ATP8, (FIG. 59C and FIG. 59G) R8-ATP8+10-ATP8, (FIG. 59D and FIG. 59H) 9-ND51+R13-ND51, or (FIG. 59E and FIG. 59I) 12-ND51+R13-ND51. For FIGS. 59B-59E, values and errors reflect the mean±s.d. of n=3 independent biological replicates. The editing efficiencies shown are for the most efficiently edited C•G within the spacing region.

FIGS. 60A-60E: Design of ZF-DdCBEs at (GNN)ₙ-rich sites. Design of 3ZF, 4ZF, and 5ZF arrays at (FIG. 60A) ND1 (GNN)ₙ-rich site 1, (FIG. 60B) COX1 (GNN)ₙ-rich site 1, (FIG. 60C) COX1 (GNN)ₙ-rich site 2, (FIG. 60D) COX2 (GNN)ₙ-rich site 1, and (FIG. 60E) ND6 (GNN)ₙ-rich site 1. (GNN)ₙ sequences are underlined, and ZF-targeted DNA sequences are indicated by thick black lines vertically above or below the corresponding DNA sequence.

FIG. 61: Extension of ZF array length improves ZF-DdCBE editing efficiency, but including extended linkers is detrimental. Mitochondrial DNA base editing efficiencies of HEK293T cells treated with 3ZF+3ZF, 4ZF+4ZF, and 5ZF+5ZF ZF-v5 DdCBE pairs targeting ND1 (GNN)ₙ-rich site 1, COX1 (GNN)ₙ-rich site 1 and 2, COX2 (GNN)ₙ-rich site 1, and ND6 (GNN)ₙ-rich site 1. To generate the ZF array length series, 3ZF arrays were extended outwards away from the spacing region to create longer 4ZF or 5ZF arrays, all of which share the same split DddA positioning and therefore maintained a fixed spacing region. 4ZF-Ext+4ZF-Ext and 5ZF-Ext+5ZF-Ext reflect ZF-DdCBE pairs in which an extended linker (TGSEKP) was incorporated into each ZF array following ZF3 (the third ZF repeat) in 4ZF and 5ZF arrays, respectively. Values shown reflect the fold-change editing efficiency for the most efficiently edited C•G within the spacing region for n=3 independent biological replicates, compared to the corresponding 3ZF+3ZF pair. A single data point for 4ZF+4ZF at ND6 (GNN)ₙ-rich site 1 at a value of 16.0-fold change is omitted from the axes range for clarity.

FIGS. 62A-62K: Defining new ZF scaffolds improves ZF-DdCBE editing efficiency. FIGS. 62A-62D: Secondary structure and amino acid sequence of canonical (FIG. 62A) 3ZF, (FIG. 62B) 4ZF, (FIG. 62C) 5ZF, and (FIG. 62D) 6ZF arrays. FIG. 62E: Amino acid sequences of ZF scaffolds X1 to X8. Different beta-motif, alpha-motif, and linker-motif sequences are colored in grey. FIGS. 62F-62K: Mitochondrial DNA base editing efficiencies of HEK293T cells treated with v5 ZF-DdCBE pairs (FIG. 62F) R8-ATP8+4-ATP8, (FIG. 62G) R8-ATP8+10-ATP8, (FIG. 62H) R8-3i-ATP8+4-3i-ATP8, (FIG. 62I) R8-3i-ATP8+10-3ii-ATP8, (FIG. 62J) 9-ND51+R13-ND51, or (FIG. 62K) 12-ND51+R13-ND51 with either canonical ZF scaffold or ZF scaffolds X1 to X8. For FIGS. 62F-62K, values and errors reflect the mean±s.d. of n=3 independent biological replicates. The editing efficiencies shown are for the most efficiently edited C•G within the spacing region.

FIGS. 63A-63F: Defining new ZF scaffolds derived from the human proteome. FIGS. 63A, 63C, and 63E: Amino acid frequencies at each sequence position from (FIG. 63A) 3,356 unique beta-motifs, (FIG. 63C) 625 unique alpha-motifs, and (FIG. 63E) 549 unique linker motifs in the human proteome. FIGS. 63B, 63D, and 63F: Amino acid frequencies at each sequence position displayed as a sequence logo (top) used to define (FIG. 63B) consensus beta-motif, (FIG. 63D) consensus alpha-motif, and (FIG. 63F) consensus linker motif sequences by applying a 10% frequency cut-off at each sequence position (bottom).

FIGS. 64A-64I: Identifying new ZF scaffolds derived from the human proteome that improve ZF-DdCBE editing efficiency. FIGS. 64A-64F: Mitochondrial DNA base editing efficiencies of HEK293T cells treated with v5 ZF-DdCBE pair R8-ATP8+4-ATP8 with either canonical or X1 ZF scaffolds, or ZF scaffolds containing (FIG. 64A) consensus beta-motifs YB1 to YB24, (FIG. 64B) YB25 to YB48, (FIG. 64C) YB49 to YB72, (FIG. 64D) YB73 to YB96, (FIG. 64E) consensus alpha-motifs YA1 to YA18, or (FIG. 64F) consensus linker motifs YL1 to YL24. FIGS. 64G-64I: The editing efficiencies of (FIG. 64G) the ten top-performing consensus beta-motifs, (FIG. 64H) four top-performing consensus alpha-motifs, or (FIG. 64I) four top-performing linker motifs. For FIGS. 64A-64I, values and errors reflect the mean±s.d. of n=3 independent biological replicates. The editing efficiencies shown are for the most efficiently edited C•G within the spacing region.

FIGS. 65A-65C: Identifying new ZF scaffolds derived from ZFN268(F1) and Sp1C that improve ZF-DdCBE editing efficiency. FIG. 65A: Amino acid sequences of ZF scaffolds based on ZF scaffold X1 and containing beta-motifs derived from ZFN268(F1) and Sp1C sequences. Amino acid changes are colored in grey. FIGS. 65B-65C: Mitochondrial DNA base editing efficiencies of HEK293T cells treated with (FIG. 65B) v5 ZF-DdCBE pairs R8-3i-ATP8+4-3i-ATP8, or (FIG. 65C) R8-3i-ATP8+10-3ii-ATP8 with either canonical ZF scaffold or ZF scaffolds from KGKS to VSGRS. For FIGS. 65B-65C, values and errors reflect the mean±s.d. of n=3 independent biological replicates. The editing efficiencies shown are for the most efficiently edited C•G within the spacing region.

FIGS. 66A-66F: Optimized ZF scaffolds increase ZF-DdCBE editing efficiency. FIGS. 66A-66F: Mitochondrial DNA base editing efficiencies of HEK293T cells treated with (FIG. 66A) v5 ZF-DdCBE pairs R8-ATP8+4-ATP8, (FIG. 66B) R8-ATP8+10-ATP8, (FIG. 66C) R8-3i-ATP8+4-3i-ATP8, (FIG. 66D) R8-3i-ATP8+10-3ii-ATP8, (FIG. 66E) 9-ND51+R13-ND51, or (FIG. 66F) 12-ND51+R13-ND51 with either canonical or optimized ZF scaffolds. For FIG. 66A and FIGS. 66C-66F, values and errors reflect the mean±s.d. of n=2 independent biological replicates. For FIG. 66B, values and errors reflect the mean±s.d. of n=3 independent biological replicates. The editing efficiencies shown are for the most efficiently edited C•G within the spacing region.

FIGS. 67A-67D: DddA mutations enhance ZF-DdCBE editing efficiency. FIGS. 67A-67D: Mitochondrial DNA base editing efficiencies of HEK293T cells treated with v5 ZF-DdCBE pairs (FIG. 67A) R8-ATP8+4-ATP8, (FIG. 67B) R8-ATP8+10-ATP8, (FIG. 67C) 9-ND51+R13-ND51, or (FIG. 67D) 12-ND51+R13-ND51 containing combinations of mutations in DddA^{N} and DddA^{C}. The triple mutant T1380I, E1396K, T1413I is colored in grey. For FIGS. 67A-67D, values and errors reflect the mean±s.d. of n=3 independent biological replicates. The editing efficiencies shown are for the most efficiently edited C•G within the spacing region.

FIGS. 68A-68G: Optimized ZF scaffolds increase ZF-DdCBE editing efficiency. FIGS. 68A-68G: Mitochondrial DNA base editing efficiencies of HEK293T cells treated with v5 ZF-DdCBE pairs (FIG. 68A) G24-R1b+G32-R1b, (FIG. 68B) G22-R13+G24-R13, (FIG. 68C) G32-R6a+G21-R6a, (FIG. 68D) G36-R6c+G212-R6c, (FIG. 68E) G33-V1+G35-V1, (FIG. 68F) G22-V2+G34-V2, or (FIG. 68G) G33-V5+G36-V5 with either canonical or optimized ZF scaffolds. For FIGS. 68A-68G, values and errors reflect the mean±s.d. of n=3 independent biological replicates. The editing efficiencies shown are for the most efficiently edited C•G within the spacing region.

FIG. 69: Identifying ZF scaffolds that support the highest editing efficiency for ZFD-derived ZF-DdCBEs. Mitochondrial DNA base editing efficiencies of HEK293T cells treated with v7 ZF-DdCBE pairs ND1-Left+ND1-Right, ND2-Left+ND2-Right, ND4L-Left+ND4L-Right, ND4-Left+ND4-Right, ND5-Left+ND5-Right, ND52-Left+ND52-Right, COX1-Left+COX1-Right, COX2-Left+COX2-Right, or CYB-Left+CYB-Right with the indicated optimized ZF scaffolds. Values and errors reflect the mean±s.d. of n=3 independent biological replicates. The editing efficiencies shown are for the most efficiently edited C•G within the spacing region.

FIGS. 70A-70B: Time course of TALE-DdCBE and ZF-DdCBE editing efficiencies over time. Mitochondrial DNA base editing efficiencies of HEK293T cells treated with (FIG. 70A) TALE-DdCBE pair ND4-DdCBE, or (FIG. 70B) v5 ZF-DdCBE pair R8-3i-ATP8+4-3i-ATP8 with the indicated amount of plasmid DNA. Cells were lysed after the indicated time period. For FIGS. 70A-70B, values and errors reflect the mean±s.d. of n=2 independent biological replicates. The editing efficiencies shown are for the most efficiently edited C•G within the spacing region.

FIG. 71: Amino acid sequences immediately upstream of DddA^{N} and DddA^{C} influence non-targeted editing activity. Average non-targeted editing efficiencies within amplicon ATP8 of HEK293T cells treated with DddA^{N}-UGI and DddA^{C}-UGI preceded by the indicated sequences. Naming convention follows A/B, where A and B correspond to the amino acid sequences immediately upstream of DddA^{N} and DddA^{C}, respectively. Values reflect the mean of n=3 independent biological replicates.

FIGS. 72A-72H: DddA truncation reduces ZF-DdCBE off-target editing. FIG. 72A: Crystal structure of DddA (PDB 6U08) complexed with DddI, the natural protein inhibitor of DddA (not shown). DddA^{N} and DddA^{C} are colored in light gray and dark gray, respectively, and have N- and C-termini indicated. FIGS. 72B-72D: (FIG. 72B) C-terminal truncation of DddA^{N}, (FIG. 72C) N-terminal truncation of DddA^{C}, and (FIG. 72D) C-terminal truncation of DddA^{C} are shown with residues incrementally removed colored in white. FIGS. 72E-72H: (FIG. 72E and FIG. 72G) On-target and (FIG. 72F and FIG. 72H) average off-target editing efficiencies within amplicon ATP8 of HEK293T cells treated with canonical v7 ZF-DdCBE pair R8-3i-ATP8+4-3i-ATP8 or variants containing DddA^{N} and DddA^{C} truncations. For FIGS. 72E-72H, values reflect the mean of n=3 independent biological replicates. The on-target editing efficiencies shown are for the most efficiently edited C•G within the spacing region.

FIGS. 73A-73B: Shifting the position of the canonical G1397 split site within DddA. FIG. 73A: On-target and average off-target editing efficiencies within amplicon ATP8 of HEK293T cells treated with canonical v7 ZF-DdCBE pair R8-3i-ATP8+4-3i-ATP8 (indicated with an arrow) or variants containing C-terminally extended DddA^{N} and N-terminally truncated DddA^{C}. FIG. 73B: Mitochondrial DNA base editing efficiencies of HEK293T cells treated with only a single ZF-DdCBE half (R8-3i-ATP8 from ZF-DdCBE pair R8-3i-ATP8+4-3i-ATP8) carrying canonical DddA^{N} or C-terminally extended DddA^{N} variants. Naming convention C+X signifies DddA_{C+X}^{N}. For FIG. 73A, values reflect the mean of n=3 independent biological replicates. For FIG. 73B, values and errors reflect the mean±s.d. of n=3 independent biological replicates. The editing efficiencies shown are for the most efficiently edited C•G within the spacing region.

FIGS. 74A-74C: Introducing negative charge at the termini of DddA or capping with catalytically inactivated DddA^{N}. Architectures of canonical ZF-DdCBEs and ZF-DdCBE variants containing a ZF array, Gly/Ser-rich flexible linker, split DddA deaminase, and UGI (N-terminal mitochondrial targeting signal, FLAG tag, and nuclear export signals are not shown). FIG. 74A: ZF-DdCBE variants are shown in which three, six, or nine residues in the 13-amino acid Gly/Ser-rich flexible linker upstream of DddA^{N} and DddA^{C} were mutated to either Glu (E) or Asp (D) residues. ZF-DdCBE variants are also shown in which three, six, or nine Glu (E) or Asp (D) residues were inserted into the Gly/Ser-rich flexible linker downstream of DddA^{N}. FIG. 74B: Off-target editing efficiencies within mitochondrial off-target amplicon ATP8 of HEK293T cells treated with individual components of the v7 ZF-DdCBE architecture, with or without the DddA catalytically inactivating E1347A mutation. FIG. 74C: ZF-DdCBE variants are shown in which dDddA^{N} was fused downstream of DddA^{C} using Gly/Ser-rich flexible linkers, either before or after the UGI domain.

FIGS. 75A-75D: Combining approaches to reduce ZF-DdCBE off-target editing. FIG. 75A: On-target and average off-target editing efficiencies within amplicon ATP8 of HEK293T cells treated with canonical v7 ZF-DdCBE pair R8-3i-ATP8+4-3i-ATP8 (indicated with an arrow) or (FIG. 75A) variants containing one (grey) or two (black) DddA^{C} point mutations from the following set: [K5A, R6A, G7A, T9A, V14A, P25A, T12K, V14K, N18K, P25K], (FIG. 75B) variants containing one or two DddA^{C} point mutations from the following set: [K5A, R6A, G7A, T9A, V14A, P25A, T12K, V14K, N18K, P25K], in combination with either DddA^{N} or DddA_{CΔ3}^{N}, (FIG. 75C) variants containing one or two DddA^{C} point mutations from the following set: [R6A, G7A, T9A, V14A, P25A, T12K, V14K, N18K, P25K], in combination with either DddA^{N} and DddA_{NΔ5}^{C}, or DddA_{CΔ3}^{N} and DddA_{NΔC}^{C}, (FIG. 75D) variants containing one, two or three changes in total, selected from any of the four approaches of single point mutations, truncations, electrostatic repulsion, and dDddA^{N} capping. For FIGS. 75A-75D, values reflect the mean of n=3 independent biological replicates. The on-target editing efficiencies shown are for the most efficiently edited C•G within the spacing region.

FIGS. 76A-76G: v8^{HS} ZF-DdCBE variants reduce off-target editing. (FIGS. 76A-76G) On-target and average off-target editing efficiencies of HEK293T cells treated with v7 (indicated with an arrow), v8, or v8^{HS1} to v8^{HS5} ZF-DdCBE pairs (FIG. 76A) G24-R1b+G32-R1b, (FIG. 76B) G22-R13+G24-R13, (FIG. 76C) G32-R6a+G21-R6a, (FIG. 76D) G36-R6c+G212-R6c, (FIG. 76E) G33-V1+G35-V1, (FIG. 76F) G22-V2+G34-V2, or (FIG. 76G) G33-V5+G36-V5. For FIGS. 76A-76G, values reflect the mean of n=3 independent biological replicates. The on-target editing efficiencies shown are for the most efficiently edited C•G within the spacing region.

FIGS. 77A-77I: Comparison between v8^{HS1} ZF-DdCBEs and ZFDs. FIGS. 77A-77I: On-target and average off-target editing efficiencies of HEK293T cells treated with ZFDs (indicated with an arrow), v7, v8, or v8^{HS1} ZF-DdCBE pairs (FIG. 77A) ND1-Left+ND1-Right, (FIG. 77B) ND2-Left+ND2-Right, (FIG. 77C) ND4L-Left+ND4L-Right, (FIG. 77D) ND4-Left+ND4-Right, (FIG. 77E) ND5-Left+ND5-Right, (FIG. 77F) ND52-Left+ND52-Right, (FIG. 77G) COX1-Left+COX1-Right, (FIG. 77H) COX2-Left+COX2-Right, or (FIG. 77I) CYB-Left+CYB-Right. For FIGS. 77A-77G, values reflect the mean of n=3 independent biological replicates. The on-target editing efficiencies shown are for the most efficiently edited C•G within the spacing region.

FIGS. 78A-78C: Optimized ZF-DdCBEs install m.8340G>A in HEK293T cells. FIG. 78A: Design of 3ZF arrays for ZF-DdCBE-mediated installation of m.8340G>A in human MT-TK. ZF-targeted DNA sequences are indicated by thick black lines vertically above or below the corresponding DNA sequence, and the target cytosine is colored light gray. FIG. 78B: Mitochondrial DNA base editing efficiencies of HEK293T cells treated with v7 ZF-DdCBE pairs with the indicated split DddA orientation (DddA^{N}/DddA^{C} signifies that the left ZF array is fused to DddA^{N} and the right ZF array is fused to DddA^{C}). FIG. 78C: Mitochondrial DNA base editing efficiencies of HEK293T cells treated with 3ZF+3ZF v7^{AGKS} ZF-DdCBE pair G21-MT-TK+G23-MT-TK or variants with the left and right ZF array extended to 4ZF or 5ZF as indicated. For FIG. 78B and FIG. 78C, values and errors reflect the mean±s.d. of n=3 independent biological replicates. The on-target editing efficiencies shown are for the most efficiently edited C•G within the spacing region.

FIGS. 79A-79G: Optimized ZF-DdCBEs install m.7743G>A in C2C12 cells. FIG. 79A: 3ZF arrays for ZF-DdCBEs designed to install m.7743G>A in mouse Mt-tk. ZF-targeted DNA sequences are indicated by thick black lines vertically above or below the corresponding DNA sequence, and the target cytosine is colored light gray. FIGS. 79B, 79D, and 79F: Mitochondrial DNA base editing efficiencies of C2C12 cells treated with (FIG. 79B) the top 27 performing v7 ZF-DdCBE pairs from the initial 3ZF+3ZF panel designed to install m.7743G>A, (FIG. 79D) the top 12 performing extended v7 ZF-DdCBE pairs designed to install m.7743G>A, (FIG. 79F) the v7 ZF-DdCBE pair LT51-Mt-tk+RB38-Mt-tk with the indicated optimized ZF scaffolds. FIG. 79C: Extension of ZF arrays from 3ZF to 4ZF, 5ZF, or 6ZF (adding additional ZF repeats to the ZF arrays extending away from the spacing region in order to maintain a fixed deaminase positioning) to test the effects of ZF extension on ZF-DdCBE editing efficiency. FIG. 79E: Mitochondrial DNA base editing efficiencies of C2C12 cells plated on either poly-D-lysine- or collagen-coated plates treated with the indicated ZF-DdCBE pairs. FIG. 79G: On-target and average off-target editing efficiencies of C2C12 cells treated with v7 (indicated with an arrow), v8, or v8^{HS1} to v8^{HS5} ZF-DdCBE pair LT51-Mt-tk+RB38-Mt-tk. For FIGS. 79D-79F, values and errors reflect the mean±s.d. of n=3 independent biological replicates. For FIG. 79G, values reflect the mean of n=3 independent biological replicates. The on-target editing efficiencies shown are for the most efficiently edited C•G within the spacing region. For FIGS. 79D-79E, all ZF-DdCBE pairs use the split DddA orientation DddA^{C}/DddA^{N}.

FIGS. 80A-80G: Optimized ZF-DdCBEs install m.3177G>A in C2C12 cells. FIG. 80A: 3ZF arrays for ZF-DdCBEs designed to install m.3177G>A in mouse Nd1. ZF-targeted DNA sequences are indicated by thick black lines vertically above or below the corresponding DNA sequence, and the target cytosine is colored light gray. FIGS. 80B, 80C, and 80E: Mitochondrial DNA base editing efficiencies of C2C12 cells treated with (FIG. 80B) the top 26 performing v7 ZF-DdCBE pairs from the initial 3ZF+3ZF panel designed to install m.3177G>A, (FIG. 80C) the top 18 performing extended v7 ZF-DdCBE pairs designed to install m.3177G>A, (FIG. 80E) the v7 ZF-DdCBE pair LB510-Nd1+RB54-Nd1 with the indicated optimized ZF scaffolds. FIG. 80D: Mitochondrial DNA base editing efficiencies of C2C12 cells plated on either poly-D-lysine- or collagen-coated plates treated with the indicated ZF-DdCBE pairs. FIG. 80F: On-target and average off-target editing efficiencies of C2C12 cells treated with v7 (indicated with an arrow), v8, or v8^{HS1} to v8^{HS5} ZF-DdCBE pair LB510-Nd1+RB54-Nd1. FIG. 80G: The m.3177G>A mutation in mouse Nd1 creates a missense E143K mutation. For FIGS. 80B-80E, values and errors reflect the mean±s.d. of n=3 independent biological replicates. For FIG. 80F, values reflect the mean of n=3 independent biological replicates. The on-target editing efficiencies shown are for the most efficiently edited C•G within the spacing region. For FIGS. 80C-80D, all ZF-DdCBE pairs use the split DddA orientation DddA^{C}/DddA^{N}.

FIGS. 81A-81C: Converting mitochondrial ZF-DdCBEs into nuclear ZF-DdCBEs. FIGS. 81A-81C: 3ZF arrays for ZF-DdCBEs designed to edit mitochondrial sites, or nuclear sites with high sequence similarity. ZF-targeted DNA sequences are indicated by thick black lines vertically above or below the corresponding DNA sequence, spacing regions are marked with arrows, and the target cytosine(s) edited in mitochondrial DNA with high efficiency are colored light gray.

FIGS. 82A-82B: Correction of a nuclear disease-causing mutation using ZF-DdCBEs. FIG. 82A: 3ZF arrays for ZF-DdCBEs designed to correct human HBB-28(A>G). ZF-targeted DNA sequences are indicated by thick black lines vertically above or below the corresponding DNA sequence, and the target cytosine is colored light gray. FIG. 82B: Mitochondrial DNA base editing efficiencies of HEK293T-HBB cells nuclear ZF-DdCBE pairs designed to correct HBB-28(A>G). All ZF-DdCBE pairs use the split DddA orientation DddA^{N}/DddA^{C}. For FIG. 82B, values and errors reflect the mean±s.d. of n=3 independent biological replicates.

FIGS. 83A-83F: Off-target editing analysis of mice treated with AAV-Mt-tk. FIGS. 83A-83F: Off-target editing efficiencies within mitochondrial off-target amplicon (FIG. 83A) OT1, (FIG. 83B) OT3, (FIG. 83C) OT4, (FIG. 83D) OT10, (FIG. 83E) OT11, or (FIG. 83F) OT12 of tissue samples from mice treated with buffer, dAAV-Mt-tk or AAV-Mt-tk. Values reflect the mean of n=4, 4, and 7 for mice treated with buffer, AAV-Mt-tk, or dAAV-Mt-tk, respectively.

FIGS. 84A-84F: Off-target editing analysis of mice treated with AAV-Nd1. FIGS. 84A-84F: Off-target editing efficiencies within mitochondrial off-target amplicon (FIG. 84A) OT2, (FIG. 84B) OT3, (FIG. 84C) OT5, (FIG. 84D) OT6, (FIG. 84E) OT9, or (FIG. 84F) OT12 of tissue samples from mice treated with buffer or AAV-Nd1. Values reflect the mean of n=4 and 7 for mice treated with buffer or AAV-Nd1, respectively.

FIGS. 85A-85D: Configurations and DNA sequences of spacing regions for the ZF-DdCBE pairs described herein. FIG. 85A: Initial mitochondrial ZF-DdCBE pairs used to establish v1 to v8 architectural improvements. FIG. 85B: Additional mitochondrial ZF-DdCBE pairs used to validate optimized architectures and HS variants. FIG. 85C: ZFD-derived mitochondrial ZF-DdCBE pairs. FIG. 85D: Nuclear ZF-DdCBE pairs. For each site the DNA spacing region, split DddA orientation, ZF array lengths, and ZF-targeted DNA strands (LT, LB, RT, RB=left top, left bottom, right top, right bottom, respectively) are shown, and the cytosine with the highest editing efficiency is colored in light gray. ZF-DdCBE naming convention follows A+B where A and B specify the left and right ZF, respectively. Nucleotide numbering starts with the first 5′-nucleotide in the spacing region designated position 1. For R8-ATP8+4-ATP8, nucleotide C5 has the highest editing efficiency.

FIGS. 86A-86C: ZF-DdCBEs correct the MELAS-causing pathogenic mutation in cultured cells in vitro. FIG. 86A: The m.3243A>G mutation in human MT-TL1 alters the D-loop of mt-tRNA^{Leu(UUR)}. FIGS. 86B-86C: Mitochondrial DNA base editing efficiencies of (FIG. 86B) HEK293T cells or (FIG. 86C) RN164 cybrid 143BTK⁻ cells treated with an optimized ZF-DdCBE pair designed to correct m.3243A>G. Values and errors reflect the mean±s.d. of n=3 independent biological replicates. For each site, the DNA spacing region, split DddA orientation, ZF array lengths, and ZF-targeted DNA strands (LT, RB=left top, right bottom, respectively) are shown, and the cytosine with highest editing efficiency is colored in light gray.

FIGS. 87A-87C: Correction of a mitochondrial disease-causing mutation using ZF-DdCBEs. FIG. 87A: 3ZF arrays for ZF-DdCBEs designed to correct m.3243A>G in human MT-TL1. ZF-targeted DNA sequences are indicated by thick black lines vertically above or below the corresponding DNA sequence, and the target cytosine is colored light gray. FIG. 87B: mtDNA base editing efficiencies of HEK293T cells (encoding wild-type MT-TL1, which lacks the m.3243A>G mutation) treated with v7 ZF-DdCBE pairs designed to correct m.3243A>G. Editing of the adjacent base at position m.3242 (CTC context) is considered a proxy for on-target editing activity. FIG. 87C: mtDNA base editing efficiencies of RN164 cybrid 143BTK− cells homoplasmic for m.3243A>G treated with v7 ZF-DdCBE pair MT-TL1•pB7-LT32/pB6N-RB6458 or variants containing additional mutations in DddAN. For FIG. 87B, values and errors reflect the mean±s.d. of n=3 independent biological replicates. For FIG. 87C, values and errors reflect the mean±s.d. of n=2 independent biological replicates.

### DEFINITIONS

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2^{nd} ed. 1994); The Cambridge Dictionary of Science and Technology (Walker ed., 1988); The Glossary of Genetics, 5^{th} Ed., R. Rieger et al. (eds.), Springer Verlag (1991); and Hale & Marham, The Harper Collins Dictionary of Biology (1991). As used herein, the following terms have the meanings ascribed to them unless specified otherwise.

### AAV

An “adeno-associated virus” or “AAV” is a virus which infects humans and some other primate species. The wild-type AAV genome is a single-stranded deoxyribonucleic acid (ssDNA), either positive- or negative-sensed. The genome comprises two inverted terminal repeats (ITRs), one at each end of the DNA strand, and two open reading frames (ORFs): rep and cap between the ITRs. The rep ORF comprises four overlapping genes encoding Rep proteins required for the AAV life cycle. The cap ORF comprises overlapping genes encoding capsid proteins: VP1, VP2 and VP3, which interact together to form the viral capsid. VP1, VP2 and VP3 are translated from one mRNA transcript, which can be spliced in two different manners: either a longer or shorter intron can be excised, resulting in the formation of two isoforms of mRNAs: a ˜2.3 kb- and a ˜2.6 kb-long mRNA isoform. The capsid forms a supramolecular assembly of approximately 60 individual capsid protein subunits into a non-enveloped, T-1 icosahedral lattice capable of protecting the AAV genome. The mature capsid is composed of VP1, VP2, and VP3 (molecular masses of approximately 87, 73, and 62 kDa respectively) in a ratio of about 1:1:10.

rAAV particles may comprise a nucleic acid vector (e.g., a recombinant genome), which may comprise at a minimum: (a) one or more heterologous nucleic acid regions comprising a sequence encoding a protein or polypeptide of interest (e.g., a split Cas9 or split nucleobase) or an RNA of interest (e.g., a gRNA), or one or more nucleic acid regions comprising a sequence encoding a Rep protein; and (b) one or more regions comprising inverted terminal repeat (ITR) sequences (e.g., wild-type ITR sequences or engineered ITR sequences) flanking the one or more nucleic acid regions (e.g., heterologous nucleic acid regions). In some embodiments, the nucleic acid vector is between 4 kb and 5 kb in size (e.g., 4.2 to 4.7 kb in size). In some embodiments, the nucleic acid vector further comprises a region encoding a Rep protein. In some embodiments, the nucleic acid vector is circular. In some embodiments, the nucleic acid vector is single-stranded. In some embodiments, the nucleic acid vector is double-stranded. In some embodiments, a double-stranded nucleic acid vector may be, for example, a self-complimentary vector that contains a region of the nucleic acid vector that is complementary to another region of the nucleic acid vector, initiating the formation of the double-strandedness of the nucleic acid vector.

### Adenosine Deaminase

As used herein, the term “adenosine deaminase” or “adenosine deaminase domain” refers to a protein or enzyme that catalyzes a deamination reaction of an adenosine (or adenine). The terms are used interchangeably. In certain embodiments, the disclosure provides base editor fusion proteins comprising one or more adenosine deaminase domains (for example, fused to any of the zinc finger domain-containing proteins provided herein). For instance, an adenosine deaminase domain may comprise a heterodimer of a first adenosine deaminase and a second deaminase domain, connected by a linker. Adenosine deaminases (e.g., engineered adenosine deaminases or evolved adenosine deaminases) provided herein may be enzymes that convert adenine (A) to inosine (I) in DNA or RNA. Such adenosine deaminase can lead to an A:T to G:C base pair conversion. In some embodiments, the deaminase is a variant of a naturally occurring deaminase from an organism. In some embodiments, the deaminase does not occur in nature. For example, in some embodiments, the deaminase is at least 50%, at least 55%, at least 60%, at least 65%, at least 70%, at least 75% at least 80%, at least 85%, at least 90%, at least 95%, at least 96%, at least 97%, at least 98%, at least 99%, or at least 99.5% identical to a naturally-occurring deaminase.

In some embodiments, the adenosine deaminase is derived from a bacterium, such as, E. coli, S. aureus, S. typhi, S. putrefaciens, H. influenzae, or C. crescentus. In some embodiments, the adenosine deaminase is a TadA deaminase. In some embodiments, the TadA deaminase is an E. coli TadA deaminase (ecTadA). In some embodiments, the TadA deaminase is a truncated E. coli TadA deaminase. For example, the truncated ecTadA may be missing one or more N-terminal amino acids relative to a full-length ecTadA. In some embodiments, the truncated ecTadA may be missing 1, 2, 3, 4, 5, 6, 7, 8, 9, 10, 11, 12, 13, 14, 15, 6, 17, 18, 19, or 20 N-terminal amino acid residues relative to the full length ecTadA. In some embodiments, the truncated ecTadA may be missing 1, 2, 3, 4, 5, 6, 7, 8, 9, 10, 11, 12, 13, 14, 15, 6, 17, 18, 19, or 20 C-terminal amino acid residues relative to the full length ecTadA. In some embodiments, the ecTadA deaminase does not comprise an N-terminal methionine. Reference is made to U.S. Patent Publication No. 2018/0073012, published Mar. 15, 2018, which is incorporated herein by reference.

### Base Editing

“Base editing” refers to genome editing technology that involves the conversion of a specific nucleic acid base into another at a targeted genomic locus (e.g., including in a mtDNA). In certain embodiments, this can be achieved without requiring double-stranded DNA breaks (DSB), or single stranded breaks (i.e., nicking). To date, other genome editing techniques, including CRISPR-based systems, begin with the introduction of a DSB at a locus of interest. Subsequently, cellular DNA repair enzymes mend the break, commonly resulting in random insertions or deletions (indels) of bases at the site of the DSB. However, when the introduction or correction of a point mutation at a target locus is desired rather than stochastic disruption of the entire gene, these genome editing techniques are unsuitable, as correction rates are low (e.g., typically 0.1% to 5%), with the major genome editing products being indels. In order to increase the efficiency of gene correction without simultaneously introducing random indels, the present inventors previously modified the CRISPR/Cas9 system to directly convert one DNA base into another without DSB formation. See, Komor, A. C., et al., Programmable editing of a target base in genomic DNA without double-stranded DNA cleavage. Nature 533, 420-424 (2016), the entire contents of which is incorporated by reference herein.

### Base Editor

The term “base editor (BE)” as used herein, refers to an agent comprising a polypeptide that is capable of making a modification to a base (e.g., A, T, C, G, or U) within a nucleic acid sequence (e.g., mtDNA) that converts one base to another (e.g., A to G, A to C, A to T, C to T, C to G, C to A, G to A, G to C, G to T, T to A, T to C, T to G). In some embodiments, the BE refers to those fusion proteins described herein which are capable of modifying bases directly in mitochondrial DNA. Such BEs can also be referred to herein as “mtDNA base editors” or “mtDNA BEs.” Such BEs can refer to those fusion proteins comprising a programmable DNA binding protein (“pDNAbp”) (e.g., any of the zinc finger domain-containing proteins provided herein, including mitoZFPs, or a CRISPR/Cas9) and a deaminase (such as a double-stranded DNA deaminase (“DddA”)) to precisely install nucleotide changes and/or correct pathogenic mutations in DNA, including mtDNA, rather than destroying the mtDNA with double-strand breaks (DSBs).

In some embodiments, the base editors contemplated herein comprise any of the zinc finger domain-containing proteins provided herein. In some embodiments, the base editors contemplated herein comprise any of the DddA variants provided herein.

In some embodiments, the base editors contemplated herein comprise a nuclease-inactive Cas9 (dCas9) fused to a deaminase which binds a nucleic acid in a guide RNA-programmed manner via the formation of an R-loop, but does not cleave the nucleic acid. For example, the dCas9 domain of the fusion protein may include a D10A and a H840A mutation (which renders Cas9 capable of cleaving only one strand of a nucleic acid duplex), as described in PCT/US2016/058344, which published as WO 2017/070632 on Apr. 27, 2017, and is incorporated herein by reference in its entirety. The DNA cleavage domain of S. pyogenes Cas9 includes two subdomains, the HNH nuclease subdomain and the RuvC1 subdomain. The HNH subdomain cleaves the strand complementary to the gRNA (the “targeted strand,” or the strand in which editing or deamination occurs), whereas the RuvC1 subdomain cleaves the non-complementary strand containing the PAM sequence (the “non-edited strand”). The RuvC1 mutant D10A generates a nick in the targeted strand, while the HNH mutant H840A generates a nick on the non-edited strand (see Jinek et al., Science, 337:816-821(2012); Qi et al., Cell. 28; 152(5):1173-83 (2013)).

BEs that convert a C to T, in some embodiments, comprise a cytidine deaminase (e.g., a double-stranded DNA deaminase or DddA). A “cytidine deaminase” (including those DddAs disclosed herein) refers to an enzyme that catalyzes the chemical reaction “cytosine+H₂O→uracil+NH₃” or “5-methyl-cytosine+H₂O→thymine+NH₃.” As it may be apparent from the reaction formula, such chemical reactions result in a C to U/T nucleobase change. In the context of a gene, such a nucleotide change, or mutation, may in turn lead to an amino acid change in the protein, which may affect the protein's function, e.g., loss-of-function or gain-of-function. In some embodiments, the C to T nucleobase editor comprises a zinc finger protein fused to a cytidine deaminase. In some embodiments, the cytidine deaminase domain is fused to the N-terminus of the zinc finger protein, or to the C-terminus of the zinc finger protein. In some embodiments, the C to T nucleobase editor comprises a Cas9 protein (e.g., an nCas9 or dCas9 protein) fused to a cytidine deaminase. In some embodiments, the cytidine deaminase is fused to the N-terminus of the Cas9 protein, or to the C-terminus of the Cas9 protein.

In some embodiments, the nucleobase editor further comprises a domain that inhibits uracil glycosylase, and/or a nuclear localization signal.

Cas9 domains used in base editing have been described in the following references, the contents of which may be applied in the instant disclosure to modify and/or include in BEs described herein, which can target mtDNA, e.g., in Rees & Liu, Nat Rev Genet. 2018; 19(12):770-788 and Koblan et al., Nat Biotechnol. 2018; 36(9):843-846; as well as U.S. Patent Publication No. 2018/0073012, published Mar. 15, 2018, which issued as U.S. Pat. No. 10,113,163; on Oct. 30, 2018; U.S. Patent Publication No. 2017/0121693, published May 4, 2017, which issued as U.S. Pat. No. 10,167,457 on Jan. 1, 2019; International Publication No. WO 2017/070633, published Apr. 27, 2017; U.S. Patent Publication No. 2015/0166980, published Jun. 18, 2015; U.S. Pat. No. 9,840,699, issued Dec. 12, 2017; U.S. Pat. No. 10,077,453, issued Sep. 18, 2018; International Publication No. WO 2019/023680, published Jan. 31, 2019; International Publication No. WO 2018/0176009, published Sep. 27, 2018, International Application No PCT/US2019/033848, filed May 23, 2019, International Application No. PCT/US2019/47996, filed Aug. 23, 2019; International Application No. PCT/US2019/049793, filed Sep. 5, 2019; U.S. Provisional Application No. 62/835,490, filed Apr. 17, 2019; International Application No. PCT/US2019/61685, filed Nov. 15, 2019; International Application No. PCT/US2019/57956, filed Oct. 24, 2019; U.S. Provisional Application No. 62/858,958, filed Jun. 7, 2019; International Publication No. PCT/US2019/58678, filed Oct. 29, 2019, the contents of each of which are incorporated herein by reference in their entireties.

Exemplary adenine and cytosine base editors are also described in Rees & Liu, Base editing: precision chemistry on the genome and transcriptome of living cells, Nat. Rev. Genet. 2018; 19(12):770-788; as well as U.S. Patent Publication No. 2018/0073012, published Mar. 15, 2018, which issued as U.S. Pat. No. 10,113,163, on Oct. 30, 2018; U.S. Patent Publication No. 2017/0121693, published May 4, 2017, which issued as U.S. Pat. No. 10,167,457 on Jan. 1, 2019; International Publication No. WO 2017/070633, published Apr. 27, 2017; U.S. Patent Publication No. 2015/0166980, published Jun. 18, 2015; U.S. Pat. No. 9,840,699, issued Dec. 12, 2017; and U.S. Pat. No. 10,077,453, issued Sep. 18, 2018, PCT Application PCT/US2017/045381, filed Aug. 3, 2017, which published as WO 2018/027078, and PCT Application No. PCT/US2019/033848, which published as WO 2019/226953, each of which is herein incorporated by reference. Any of the deaminase components of these adenine or cytidine BEs could be modified using a method of directed evolution (e.g., PACE or PANCE) to obtain a deaminase which may use double-stranded DNA as a substrate, and thus, which could be used in the BEs described herein, which are intended, for example, for use in conducting base editing directly on mtDNA, i.e., on a double-stranded DNA target.

### Cas9

The term “Cas9” or “Cas9 nuclease” refers to an RNA-guided nuclease comprising a Cas9 domain, or a fragment thereof (e.g., a protein comprising an active or inactive DNA cleavage domain of Cas9, and/or the gRNA binding domain of Cas9). A “Cas9 domain” as used herein, is a protein fragment comprising an active or inactive cleavage domain of Cas9 and/or the gRNA binding domain of Cas9. A “Cas9 protein” is a full length Cas9 protein. A Cas9 nuclease is also referred to sometimes as a casn1 nuclease or a CRISPR (Clustered Regularly Interspaced Short Palindromic Repeat)-associated nuclease. CRISPR is an adaptive immune system that provides protection against mobile genetic elements (viruses, transposable elements, and conjugative plasmids). CRISPR clusters contain spacers, sequences complementary to antecedent mobile elements, and target invading nucleic acids. CRISPR clusters are transcribed and processed into CRISPR RNA (crRNA). In type II CRISPR systems, correct processing of pre-crRNA requires a trans-encoded small RNA (tracrRNA), endogenous ribonuclease 3 (rnc), and a Cas9 domain. The tracrRNA serves as a guide for ribonuclease III-aided processing of pre-crRNA. Subsequently, Cas9/crRNA/tracrRNA endonucleolytically cleaves linear or circular dsDNA target complementary to the spacer. The target strand not complementary to crRNA is first cut endonucleolytically, then trimmed 3′-5′ exonucleolytically. In nature, DNA-binding and cleavage typically requires protein and both RNAs. However, single guide RNAs (“sgRNA”, or simply “gRNA”) can be engineered so as to incorporate aspects of both the crRNA and tracrRNA into a single RNA species. See, e.g., Jinek M., Chylinski K., Fonfara I., Hauer M., Doudna J. A., Charpentier E. Science 337:816-821(2012), the entire contents of which are hereby incorporated by reference. Cas9 recognizes a short motif in the CRISPR repeat sequences (the PAM or protospacer adjacent motif) to help distinguish self versus non-self. Cas9 nuclease sequences and structures are well known to those of skill in the art (see, e.g., “Complete genome sequence of an M1 strain of Streptococcus pyogenes.” Ferretti et al., J. J., McShan W. M., Ajdic D. J., Savic D. J., Savic G., Lyon K., Primeaux C., Sezate S., Suvorov A. N., Kenton S., Lai H. S., Lin S. P., Qian Y., Jia H. G., Najar F. Z., Ren Q., Zhu H., Song L., White J., Yuan X., Clifton S. W., Roe B. A., McLaughlin R. E., Proc. Natl. Acad. Sci. U.S.A. 98:4658-4663(2001); “CRISPR RNA maturation by trans-encoded small RNA and host factor Rnase III.” Deltcheva E., Chylinski K., Sharma C. M., Gonzales K., Chao Y., Pirzada Z. A., Eckert M. R., Vogel J., Charpentier E., Nature 471:602-607(2011); and “A programmable dual-RNA-guided DNA endonuclease in adaptive bacterial immunity.” Jinek M., Chylinski K., Fonfara I., Hauer M., Doudna J. A., Charpentier E. Science 337:816-821(2012), the entire contents of each of which are incorporated herein by reference). Cas9 orthologs have been described in various species, including, but not limited to, S. pyogenes and S. thermophilus. Additional suitable Cas9 nucleases and sequences will be apparent to those of skill in the art based on this disclosure, and such Cas9 nucleases and sequences include Cas9 sequences from the organisms and loci disclosed in Chylinski, Rhun, and Charpentier, “The tracrRNA and Cas9 families of type II CRISPR-Cas immunity systems” (2013) RNA Biology 10:5, 726-737; the entire contents of which are incorporated herein by reference. In some embodiments, a Cas9 nuclease comprises one or more mutations that partially impair or inactivate the DNA cleavage domain.

A nuclease-inactivated Cas9 domain may interchangeably be referred to as a “dCas9” protein (for nuclease-“dead” Cas9). Methods for generating a Cas9 domain (or a fragment thereof) having an inactive DNA cleavage domain are known (see, e.g., Jinek et al., Science. 337:816-821(2012); Qi et al., “Repurposing CRISPR as an RNA-Guided Platform for Sequence-Specific Control of Gene Expression” (2013) Cell. 28; 152(5):1173-83, the entire contents of each of which are incorporated herein by reference). For example, the DNA cleavage domain of Cas9 is known to include two subdomains, the HNH nuclease subdomain and the RuvC1 subdomain. The HNH subdomain cleaves the strand complementary to the gRNA, whereas the RuvC1 subdomain cleaves the non-complementary strand. Mutations within these subdomains can silence the nuclease activity of Cas9. For example, the mutations D10A and H840A completely inactivate the nuclease activity of S. pyogenes Cas9 (Jinek et al., Science. 337:816-821(2012); Qi et al., Cell. 28; 152(5):1173-83 (2013)). In some embodiments, proteins comprising fragments of Cas9 are provided. For example, in some embodiments, a protein comprises one of two Cas9 domains: (1) the gRNA binding domain of Cas9; or (2) the DNA cleavage domain of Cas9. In some embodiments, proteins comprising Cas9, or fragments thereof, are referred to as “Cas9 variants.” A Cas9 variant shares homology to Cas9, or a fragment thereof. For example, a Cas9 variant is at least about 70% identical, at least about 80% identical, at least about 90% identical, at least about 95% identical, at least about 96% identical, at least about 97% identical, at least about 98% identical, at least about 99% identical, at least about 99.5% identical, at least about 99.8% identical, or at least about 99.9% identical to wild type Cas9 (e.g., SpCas9 of SEQ ID NO: 450). In some embodiments, the Cas9 variant may have 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50, or more amino acid changes compared to wild type Cas9 (e.g., SpCas9 of SEQ ID NO: 450). In some embodiments, the Cas9 variant comprises a fragment of Cas9 (e.g., a gRNA binding domain or a DNA-cleavage domain), such that the fragment is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to the corresponding fragment of wild type Cas9 (e.g., SpCas9 of SEQ ID NO: 450). In some embodiments, the fragment is at least 30%, at least 35%, at least 40%, at least 45%, at least 50%, at least 55%, at least 60%, at least 65%, at least 70%, at least 75%, at least 80%, at least 85%, at least 90%, at least 95% identical, at least 96%, at least 97%, at least 98%, at least 99%, or at least 99.5% of the amino acid length of a corresponding wild type Cas9 (e.g., SpCas9 of SEQ ID NO: 450).

The amino acid sequence of wild type SpCas9 is:

(SEQ ID NO: 450)
MDKKYSIGLDIGTNSVGWAVITDEYKVPSKKFKVLGNTDRHSIKKNLIG
ALLFDSGETAEATRLKRTARRRYTRRKNRICYLQEIFSNEMAKVDDSFF
HRLEESFLVEEDKKHERHPIFGNIVDEVAYHEKYPTIYHLRKKLVDSTD
KADLRLIYLALAHMIKFRGHFLIEGDLNPDNSDVDKLFIQLVQTYNQLF
EENPINASGVDAKAILSARLSKSRRLENLIAQLPGEKKNGLFGNLIALS
LGLTPNFKSNFDLAEDAKLQLSKDTYDDDLDNLLAQIGDQYADLFLAAK
NLSDAILLSDILRVNTEITKAPLSASMIKRYDEHHQDLTLLKALVRQQL
PEKYKEIFFDQSKNGYAGYIDGGASQEEFYKFIKPILEKMDGTEELLVK
LNREDLLRKQRTFDNGSIPHQIHLGELHAILRRQEDFYPFLKDNREKIE
KILTFRIPYYVGPLARGNSRFAWMTRKSEETITPWNFEEVVDKGASAQS
FIERMTNFDKNLPNEKVLPKHSLLYEYFTVYNELTKVKYVTEGMRKPAF
LSGEQKKAIVDLLFKTNRKVTVKQLKEDYFKKIECFDSVEISGVEDRFN
ASLGTYHDLLKIIKDKDFLDNEENEDILEDIVLTLTLFEDREMIEERLK
TYAHLFDDKVMKQLKRRRYTGWGRLSRKLINGIRDKQSGKTILDFLKSD
GFANRNFMQLIHDDSLTFKEDIQKAQVSGQGDSLHEHIANLAGSPAIKK
GILQTVKVVDELVKVMGRHKPENIVIEMARENQTTQKGQKNSRERMKRI
EEGIKELGSQILKEHPVENTQLQNEKLYLYYLQNGRDMYVDQELDINRL
SDYDVDHIVPQSFLKDDSIDNKVLTRSDKNRGKSDNVPSEEVVKKMKNY
WRQLLNAKLITQRKFDNLTKAERGGLSELDKAGFIKRQLVETRQITKHV
AQILDSRMNTKYDENDKLIREVKVITLKSKLVSDFRKDFQFYKVREINN
YHHAHDAYLNAVVGTALIKKYPKLESEFVYGDYKVYDVRKMIAKSEQEI
GKATAKYFFYSNIMNFFKTEITLANGEIRKRPLIETNGETGEIVWDKGR
DFATVRKVLSMPQVNIVKKTEVQTGGFSKESILPKRNSDKLIARKKDWD
PKKYGGFDSPTVAYSVLVVAKVEKGKSKKLKSVKELLGITIMERSSFEK
NPIDFLEAKGYKEVKKDLIIKLPKYSLFELENGRKRMLASAGELQKGNE
LALPSKYVNFLYLASHYEKLKGSPEDNEQKQLFVEQHKHYLDEIIEQIS
EFSKRVILADANLDKVLSAYNKHRDKPIREQAENIIHLFTLTNLGAPAA
FKYFDTTIDRKRYTSTKEVLDATLIHQSITGLYETRIDLSQLGGD

As used herein, the term “nCas9” or “Cas9 nickase” refers to a Cas9 or a variant thereof, which cleaves or nicks only one of the strands of a target cut site thereby introducing a nick in a double strand DNA molecule rather than creating a double strand break. This can be achieved by introducing appropriate mutations in a wild-type Cas9 which inactivates one of the two endonuclease activities of the Cas9. Any suitable mutation which inactivates one Cas9 endonuclease activity but leaves the other intact is contemplated, such as one of D10A or H840A mutations in the wild-type S. pyogenes Cas9 amino acid sequence, or a D10A mutation in the wild-type S. aureus Cas9 amino acid sequence, may be used to form the nCas9.

The amino acid sequence of SpCas9 nickase is:

(SEQ ID NO: 451)
MDKKYSIGLDIGTNSVGWAVITDEYKVPSKKFKVLGNTDRHSIKKNLIG
ALLFDSGETAEATRLKRTARRRYTRRKNRICYLQEIFSNEMAKVDDSFF
HRLEESFLVEEDKKHERHPIFGNIVDEVAYHEKYPTIYHLRKKLVDSTD
KADLRLIYLALAHMIKFRGHFLIEGDLNPDNSDVDKLFIQLVQTYNQLF
EENPINASGVDAKAILSARLSKSRRLENLIAQLPGEKKNGLFGNLIALS
LGLTPNFKSNFDLAEDAKLQLSKDTYDDDLDNLLAQIGDQYADLFLAAK
NLSDAILLSDILRVNTEITKAPLSASMIKRYDEHHQDLTLLKALVRQQL
PEKYKEIFFDQSKNGYAGYIDGGASQEEFYKFIKPILEKMDGTEELLVK
LNREDLLRKQRTFDNGSIPHQIHLGELHAILRRQEDFYPFLKDNREKIE
KILTFRIPYYVGPLARGNSRFAWMTRKSEETITPWNFEEVVDKGASAQS
FIERMTNFDKNLPNEKVLPKHSLLYEYFTVYNELTKVKYVTEGMRKPAF
LSGEQKKAIVDLLFKTNRKVTVKQLKEDYFKKIECFDSVEISGVEDRFN
ASLGTYHDLLKIIKDKDFLDNEENEDILEDIVLTLTLFEDREMIEERLK
TYAHLFDDKVMKQLKRRRYTGWGRLSRKLINGIRDKQSGKTILDFLKSD
GFANRNFMQLIHDDSLTFKEDIQKAQVSGQGDSLHEHIANLAGSPAIKK
GILQTVKVVDELVKVMGRHKPENIVIEMARENQTTQKGQKNSRERMKRI
EEGIKELGSQILKEHPVENTQLQNEKLYLYYLQNGRDMYVDQELDINRL
SDYDVDAIVPQSFLKDDSIDNKVLTRSDKNRGKSDNVPSEEVVKKMKNY
WRQLLNAKLITQRKFDNLTKAERGGLSELDKAGFIKRQLVETRQITKHV
AQILDSRMNTKYDENDKLIREVKVITLKSKLVSDFRKDFQFYKVREINN
YHHAHDAYLNAVVGTALIKKYPKLESEFVYGDYKVYDVRKMIAKSEQEI
GKATAKYFFYSNIMNFFKTEITLANGEIRKRPLIETNGETGEIVWDKGR
DFATVRKVLSMPQVNIVKKTEVQTGGFSKESILPKRNSDKLIARKKDWD
PKKYGGFDSPTVAYSVLVVAKVEKGKSKKLKSVKELLGITIMERSSFEK
NPIDFLEAKGYKEVKKDLIIKLPKYSLFELENGRKRMLASAGELQKGNE
LALPSKYVNFLYLASHYEKLKGSPEDNEQKQLFVEQHKHYLDEIIEQIS
EFSKRVILADANLDKVLSAYNKHRDKPIREQAENIIHLFTLTNLGAPAA
FKYFDTTIDRKRYTSTKEVLDATLIHQSITGLYETRIDLSQLGGD

### Cytidine Deaminase

As used herein, a “cytidine deaminase” encoded by the CDA gene is an enzyme that catalyzes the removal of an amine group from cytidine (i.e., the base cytosine when attached to a ribose ring) to uridine (C to U) and deoxycytidine to deoxyuridine (C to U). A non-limiting example of a cytidine deaminase is APOBEC1 (“apolipoprotein B mRNA editing enzyme, catalytic polypeptide 1”). Another example is AID (“activation-induced cytidine deaminase”). Under standard Watson-Crick hydrogen bond pairing, a cytosine base hydrogen bonds to a guanine base. When cytidine is converted to uridine (or deoxycytidine is converted to deoxyuridine), the uridine (or the uracil base of uridine) undergoes hydrogen bond pairing with the base adenine. Thus, a conversion of “C” to uridine (“U”) by cytidine deaminase will cause the insertion of “A” instead of a “G” during cellular repair and/or replication processes. Since the adenine “A” pairs with thymine “T”, the cytidine deaminase in coordination with DNA replication causes the conversion of an C-G pairing to a T-A pairing in the double-stranded DNA molecule.

### Deaminase

The term “deaminase” or “deaminase domain” refers to a protein or enzyme that catalyzes a deamination reaction. In some embodiments, the deaminase is an adenosine (or adenine) deaminase, which catalyzes the hydrolytic deamination of adenine or adenosine. In some embodiments, the adenosine deaminase catalyzes the hydrolytic deamination of adenine or adenosine in deoxyribonucleic acid (DNA) to inosine. In other embodiments, the deaminase is a cytidine (or cytosine) deaminase, which catalyzes the hydrolytic deamination of cytidine or cytosine. In preferred aspects, the deaminase is a double-stranded DNA deaminase, or is modified, evolved, or otherwise altered to be able to utilize double-strand DNA as a substrate for deamination.

The deaminase embraces the DddA domains described herein and defined below. The DddA is a type of deaminase, but where the activity of the deaminase is against double-stranded DNA, rather than single-stranded DNA, which is the case for deaminases prior to the present disclosure.

The deaminases provided herein may be from any organism, such as a bacterium. In some embodiments, the deaminase or deaminase domain is a variant of a naturally-occurring deaminase from an organism. In some embodiments, the deaminase or deaminase domain does not occur in nature. For example, in some embodiments, the deaminase or deaminase domain is at least 50%, at least 55%, at least 60%, at least 65%, at least 70%, at least 75% at least 80%, at least 85%, at least 90%, at least 95%, at least 96%, at least 97%, at least 98%, at least 99%, or at least 99.5% identical to a naturally-occurring deaminase.

### DNA Editing Efficiency

The term “DNA editing efficiency,” as used herein, refers to the number or proportion of intended base pairs that are edited. For example, if a base editor edits 10% of the base pairs that it is intended to target (e.g., within a cell or within a population of cells), then the base editor can be described as being 10% efficient. Some aspects of editing efficiency embrace the modification (e.g., deamination) of a specific nucleotide within DNA, without generating a large number or percentage of insertions or deletions (i.e., indels). It is generally accepted that editing while generating less than 5% indels (as measured over total target nucleotide substrates) is high editing efficiency. The generation of more than 20% indels is generally accepted as poor or low editing efficiency. Indel formation may be measured by techniques known in the art, including high-throughput screening of sequencing reads.

### DddA

The term “double-stranded DNA deaminase domain” or “DddA” (or equivalently, DddE) refers to a protein that catalyzes a deamination of a target nucleotide (e.g., C, A, G, C) in a double-stranded DNA molecule. References to DddA and double-stranded DNA deaminase are equivalent. In one embodiment, the DddA deaminates a cytidine. Deamination of cytidine results in a uracil (or deoxyuracil in the case of deoxycytidine), and through replication and/or repair processes, converts the original C:G base pair to a T:A base pair. This change can also be referred to as a “C-to-T” edit because the C of the C:G pair is converted to a T of T:A pair. DddA, when expressed naturally, can be toxic to biological systems. While the mechanism of action is not clearly documented, one rationale for the observed toxicity is that DddA's activity may cause indiscriminate deamination of cytidine in vivo on double-stranded target DNA (e.g., the cellular genome). Such indiscriminate deaminations may provoke cellular repair responses, including, but not limited to, degradation of genomic DNA. Canonical DddA was described in Mok et al., “A bacterial cytidine deaminase toxin enables CRISPR-free mitochondrial base editing,” Nature, 2020; 583(7817): 631-637 (“Mok et al., 2020”), (incorporated herein by reference). Canonical DddA was discovered in Burkholderia cenocepia and reported Mok et al. and in the Protein Data Bank as PDB ID: 6U08, which has the following full-length amino acid sequence (1427 amino acids):

>tr|A0A1V6L4E7|A0A1V6L4E7_9BURK YD repeat (Two copies) OS =
Burkholderia cenocepacia OX = 95486 GN = UE95_03830 PE = 1 SV = 1
(1427 AA-the canonical protein or “canonical DddA”)
(SEQ ID NO: 356)
MYEAARVTDPIDHTSALAGFLVGAVLGIALIAAVAFATFTCGFGVALLAGMMAGIGAQ
ALLSIGESIGKMFSSQSGNIITGSPDVYVNSLSAAYATLSGVACSKHNPIPLVAQGSTNIFI
NGRPAARKDDKITCGATIGDGSHDTFFHGGTQTYLPVDDEVPPWLRTATDWAFTLAGL
VGGLGGLLKASGGLSRAVLPCAAKFIGGYVLGEAFGRYVAGPAINKAIGGLFGNPIDVT
TGRKILLAESETDYVIPSPLPVAIKRFYSSGIDYAGTLGRGWVLPWEIRLHARDGRLWYT
DAQGRESGFPMLRAGQAAFSEADQRYLTRTPDGRYILHDLGERYYDFGQYDPESGRIA
WVRRVEDQAGQWYQFERDSRGRVTEILTCGGLRAVLDYETVFGRLGTVTLVHEDERRL
AVTYGYDENGQLASVTDANGAVVRQFAYTNGLMTSHMNALGFTSSYVWSKIEGEPRV
VETHTSEGENWTFEYDVAGRQTRVRHADGRTAHWRFDAQSQIVEYTDLDGAFYRIKY
DAVGMPVMLMLPGDRTVMFEYDDAGRIIAETDPLGRTTRTRYDGNSLRPVEVVGPDGG
AWRVEYDQQGRVVSNQDSLGRENRYEYPKALTALPSAHIDALGGRKTLEWNSLGKLV
GYTDCSGKTTRTSFDAFGRICSRENALGQRITYDVRPTGEPRRVTYPDGSSETFEYDAAG
TLVRYIGLGGRVQELLRNARGQLIEAVDPAGRRVQYRYDVEGRLRELQQDHARYTFTY
SAGGRLLTETRPDGILRRFEYGEAGELLGLDIVGAPDPHATGNRSVRTIRFERDRMGVLK
VQRTPTEVTRYQHDKGDRLVKVERVPTPSGIALGIVPDAVEFEYDKGGRLVAEHGSNGS
VIYTLDELDNVVSLGLPHDQTLQMLRYGSGHVHQIRFGDQVVADFERDDLHREVSRTQ
GRLTQRSGYDPLGRKVWQSAGIDPEMLGRGSGQLWRNYGYDAAGDLIETSDSLRGSTR
FSYDPAGRLISRANPLDRKFEEFAWDAAGNLLDDAQRKSRGYVEGNRLLMWQDLRFE
YDPFGNLATKRRGANQTQRFTYDGQDRLITVHTQDVRGVVETRFAYDPLGRRIAKTDT
AFDLRGMKLRAETKRFVWEGLRLVQEVRETGVSSYVYSPDAPYSPVARADTVMAEAL
AATVIDSAKRAARIFHFHTDPVGAPQEVTDEAGEVAWAGQYAAWGKVEATNRGVTAA
RTDQPLRFAGQYADDSTGLHYNTFRFYDPDVGRFINQDPIGLNGGANVYHYAPNPVGW
VDPWGLAGSYALGPYQISAPQLPAYNGQTVGTFYYVNDAGGLESKVFSSGGPTPYPNY
ANAGHVEGQSALFMRDNGISEGLVFHNNPEGTCGFCVNMTETLLPENAKMTVVPPEGA
IPVKRGATGETKVFTGNSNSPKSPTKGGC.

### Effective Amount

The term “effective amount,” as used herein, refers to an amount of a biologically active agent that is sufficient to elicit a desired biological response. For example, in some embodiments, an effective amount of any of the fusion proteins as described herein, or compositions thereof, may refer to the amount of the fusion proteins sufficient to edit a target nucleotide sequence (e.g., mtDNA). In some embodiments, an effective amount of any of the fusion proteins as described herein, or compositions thereof (e.g., a fusion protein comprising any of the zinc finger domain-containing proteins disclosed herein and any of the DddA variants disclosed herein) that is sufficient to induce editing of a target nucleotide, which is proximal to a target nucleic acid sequence specifically bound and edited by the fusion protein. As will be appreciated by the skilled artisan, the effective amount of an agent (e.g., a fusion protein), may vary depending on various factors such as, for example, the desired biological response on the specific allele, genome, or target site to be edited, on the cell or tissue being targeted, and on the agent being used.

### Fusion Protein

The term “fusion protein” as used herein refers to a hybrid polypeptide which comprises protein domains from at least two different proteins (e.g., a programmable DNA binding protein, such as any of the zinc finger domain-containing proteins disclosed herein, and a deaminase, such as any of the DddA variants disclosed herein). One protein may be located at the amino-terminal (N-terminal) portion of the fusion protein or at the carboxy-terminal (C-terminal) portion of the fusion protein, thus forming an “amino-terminal fusion protein” or a “carboxy-terminal fusion protein,” respectively. A protein may comprise different domains, for example, a nucleic acid binding protein (e.g., a zinc finger domain-containing protein) and a catalytic domain of a nucleic-acid editing protein (e.g., a DddA variant, or a portion of a DddA variant). Any of the proteins provided herein may be produced by any method known in the art. For example, the proteins provided herein may be produced via recombinant protein expression and purification, which is especially suited for fusion proteins comprising a peptide linker. Methods for recombinant protein expression and purification are well known, and include those described by Green and Sambrook, Molecular Cloning: A Laboratory Manual (4^{th} ed., Cold Spring Harbor Laboratory Press, Cold Spring Harbor, N.Y. (2012)), the entire contents of which are incorporated herein by reference.

### Lentiviral Vectors

Lentiviral vectors are derived from human immunodeficiency virus-1 (HIV-1). The lentiviral genome consists of single-stranded RNA that is reverse-transcribed into DNA and then integrated into the host cell genome. Lentiviruses can infect both dividing and non-dividing cells, making them attractive tools for gene therapy.

The lentiviral genome is around 9 kb in length and contains three major structural genes: gag, pol, and env. The gag gene is translated into three viral core proteins: 1) matrix (MA) proteins, which are necessary for virion assembly and infection of non-dividing cells; 2) capsid (CA) proteins, which form the hydrophobic core of the virion; and 3) nucleocapsid (NC) proteins, which protect the viral genome by coating and associating tightly with the RNA. The pol gene encodes for the viral protease, reverse transcriptase, and integrase enzymes that are essential for viral replication. The env gene encodes for the viral surface glycoproteins, which are essential for virus entry into the host cell by enabling binding to cellular receptors and fusion with cellular membranes. In some embodiments, the viral glycoprotein is derived from vesicular stomatitis virus (VSV-G). The viral genome also contains regulatory genes, including tat and rev. Tat encodes transactivators critical for activating viral transcription, while rev encodes a protein that regulates the splicing and export of viral transcripts. Tat and rev are the first proteins synthesized following viral integration and are required to accelerate production of viral mRNAs.

To improve the safety of lentivirus, the components necessary for viral production are split across multiple vectors. In some embodiments, the disclosure relates to delivery of a heterologous gene (e.g., transgene) via a recombinant lentiviral transfer vector encoding one or more transgenes of interest flanked by long terminal repeat (LTR) sequences. These LTRs are identical nucleotide sequences that are repeated hundreds or thousands of times and facilitate the integration of the transfer plasmid sequences into the host cell genome. Methods of the current disclosure also describe one or more accessory plasmids. These accessory plasmids may include one or more lentiviral packaging plasmids, which encode the pol and rev genes that are necessary for the replication, splicing, and export of viral particles. The accessory plasmids may also include a lentiviral envelope plasmid, which encodes the genes necessary for producing the viral glycoproteins that will allow the viral particle to fuse with the host cell.

### Linker

In various embodiments, the herein disclosed fusion proteins (e.g., base editors comprising, for example, any of the zinc finger domain-containing proteins and DddA variants disclosed herein) or the polypeptides that comprise the fusion proteins (e.g., the zinc finger domain-containing proteins or other pDNAbps, and DddA variants or other deaminases) may be engineered to include one or more linker sequences that join two or more polypeptides (e.g., a pDNAbp and a DddA half) to one another.

The term “linker,” as used herein, refers to a molecule linking two other molecules or moieties. The linker can be an amino acid sequence in the case of a linker joining two fusion proteins. For example, a zinc finger domain-containing protein can be fused to a first or second portion of a DddA, by an amino acid linker sequence. The linker can also be a nucleotide sequence in the case of joining two nucleotide sequences together. In other embodiments, the linker is an organic molecule, group, polymer, or chemical moiety. In some embodiments, the linker is 1-100 amino acids in length, for example, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 30-35, 35-40, 40-45, 45-50, 50-60, 60-70, 70-80, 80-90, 90-100, 100-150, or 150-200 amino acids in length. Longer linkers are also contemplated.

mitoZFP

In various embodiments, the mtDNA base editors embrace fusion proteins comprising a DddA (or inactive fragment thereof) and a mitoZFP domain. A “mitoZFP” refers to a zinc finger DNA binding protein that has been modified to comprise one or more mitochondrial targeting sequences (MTS), as described further herein.

### Mitochondrial Targeting Sequence (MTS)

In various embodiments, the base editors or the polypeptides that comprise the base editors (e.g., the pDNAbps (such as zinc finger domain-containing proteins) and DddA) disclosed herein may be engineered to include one or more mitochondrial targeting sequences (MTS) (or mitochondrial localization sequence (MLS)) that facilitate the translocation of a polypeptide into the mitochondria. Such base editors may be referred to herein as mtDNA base editors. MTS are known in the art, and exemplary sequences are provided herein. In general MTSs are short peptide sequences (about 3-70 amino acids long) that direct a newly synthesized protein to the mitochondria within a cell. It is usually found at the N-terminus and consists of an alternating pattern of hydrophobic and positively charged amino acids to form what is called an amphipathic helix. Mitochondrial localization sequences can contain additional signals that subsequently target the protein to different regions of the mitochondria, such as the mitochondrial matrix. One exemplary mitochondrial localization sequence is the mitochondrial localization sequence derived from Cox8, a mitochondrial cytochrome c oxidase subunit VIII. In some embodiments, a mitochondrial localization sequence derived from Cox8 includes the amino acid sequence: MSVLTPLLLRGLTGSARRLPVPRAKIHSL (SEQ ID NO: 357). In some embodiments, the mitochondrial localization sequence derived from Cox8 includes an amino acid sequence that is about 50%, 55%, 60%, 65%, 70%, 75%, 80%, 85%, 90% or 95% identity to SEQ ID NO: 357.

napDNAbp

In various embodiments, the base editors provided herein may comprise pDNAbps that are nucleic acid programmable (e.g., a base editor comprising a napDNAbp such as Cas9 and any of the DddA variants disclosed herein). The term “napDNAbp” which stands for “nucleic acid programmable DNA binding protein” refers to any protein that may associate (e.g., form a complex) with one or more nucleic acid molecules (i.e., which may broadly be referred to as a “napDNAbp-programming nucleic acid molecule” and includes, for example, guide RNA in the case of Cas systems) that direct or otherwise program the protein to localize to a specific target nucleotide sequence (e.g., a gene locus of a genome) that is complementary to the one or more nucleic acid molecules (or a portion or region thereof) associated with the protein, thereby causing the protein to bind to the nucleotide sequence at the specific target site. The term napDNAbp embraces CRISPR-Cas9 proteins, as well as Cas9 equivalents, homologs, orthologs, or paralogs, whether naturally occurring or non-naturally occurring (e.g., engineered or modified), and may include a Cas9 equivalent from any type of CRISPR system (e.g., type II, V, VI), including Cpf1 (a type-V CRISPR-Cas systems), C2c1 (a type V CRISPR-Cas system), C2c2 (a type VI CRISPR-Cas system), C2c3 (a type V CRISPR-Cas system), dCas9, GeoCas9, CjCas9, Cas12a, Cas12b, Cas12c, Cas12d, Cas12g, Cas12h, Cas12i, Cas13d, Cas14, Argonaute, and nCas9. Further Cas-equivalents are described in Makarova et al., “C2c2 is a single-component programmable RNA-guided RNA-targeting CRISPR effector,” Science 2016; 353 (6299), the contents of which are incorporated herein by reference. However, the nucleic acid programmable DNA binding proteins (napDNAbps) that may be used in connection with this invention are not limited to CRISPR-Cas systems. The invention embraces any such programmable protein, such as the Argonaute protein from Natronobacterium gregoryi (NgAgo), which may also be used for DNA-guided genome editing. The NgAgo-guide DNA system does not require a PAM sequence or guide RNA molecules, which means genome editing can be performed simply by the expression of generic NgAgo protein and introduction of synthetic oligonucleotides on any genomic sequence. See Gao et al., DNA-guided genome editing using the Natronobacterium gregoryi Argonaute. Nature Biotechnology 2016; 34(7):768-73, which is incorporated herein by reference.

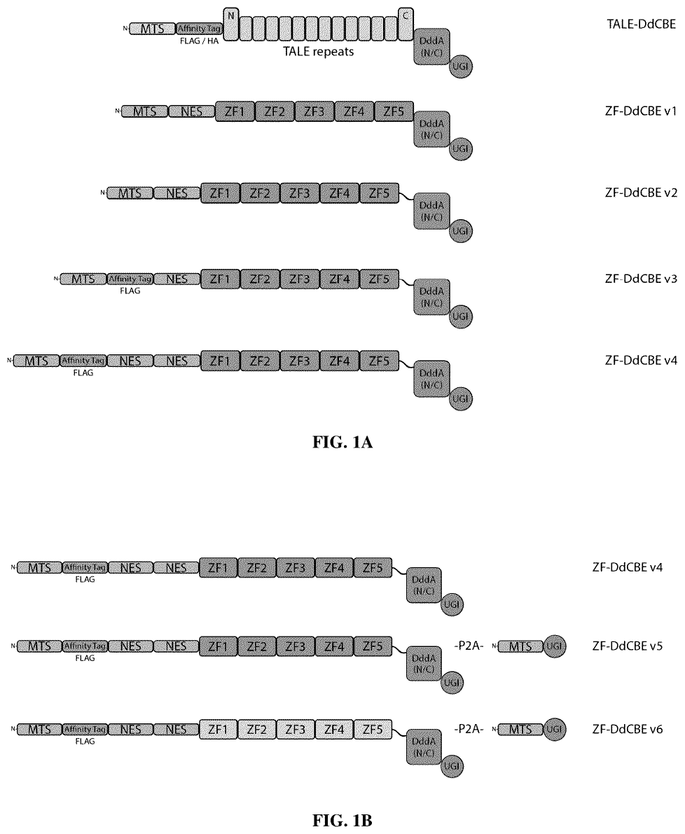

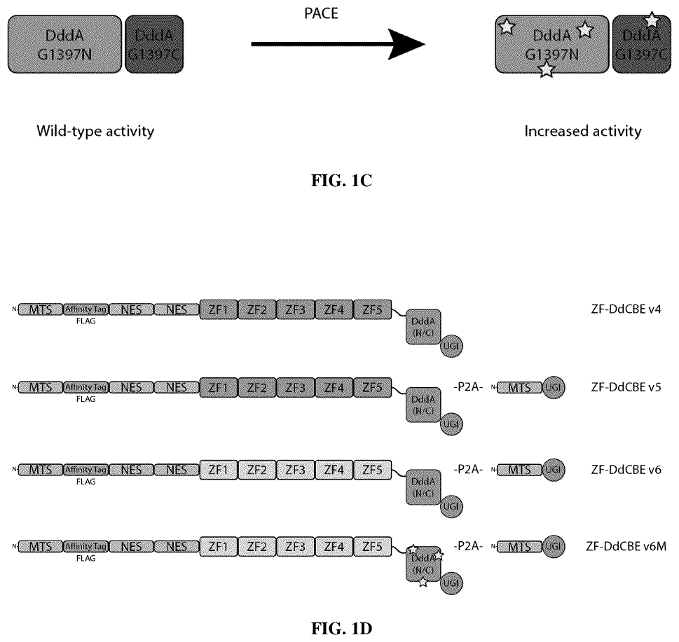

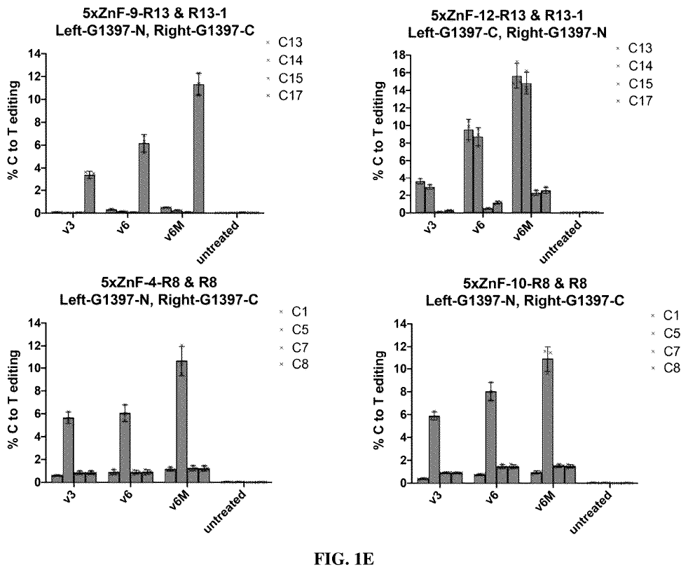

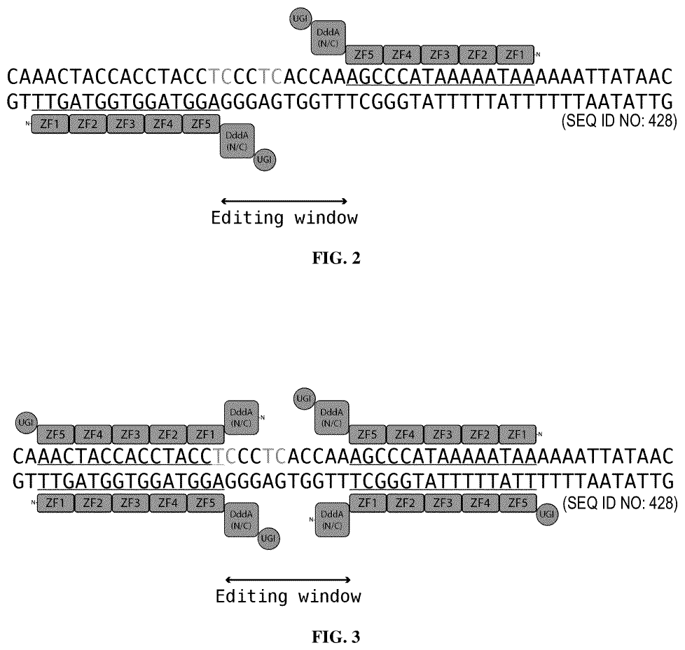

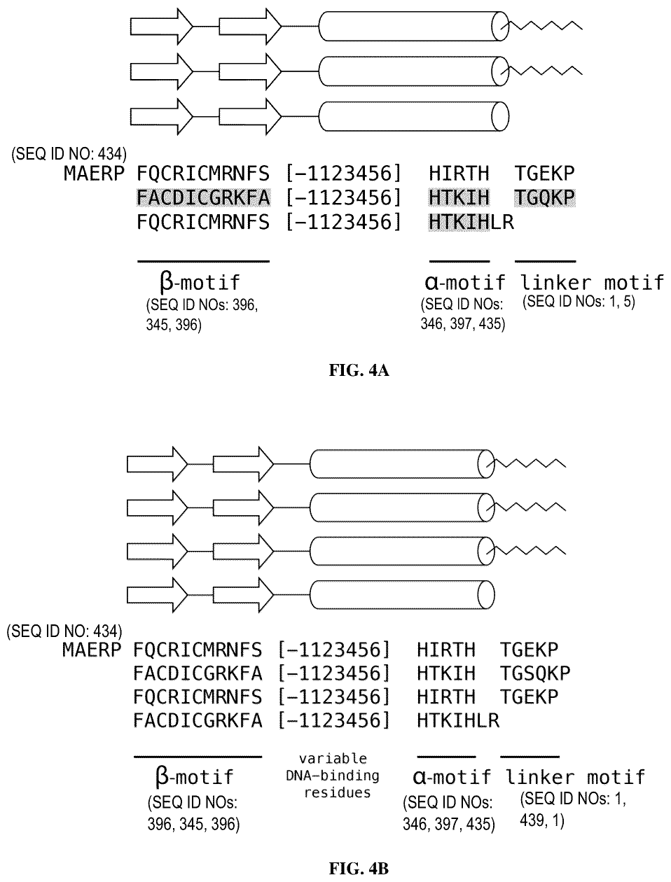

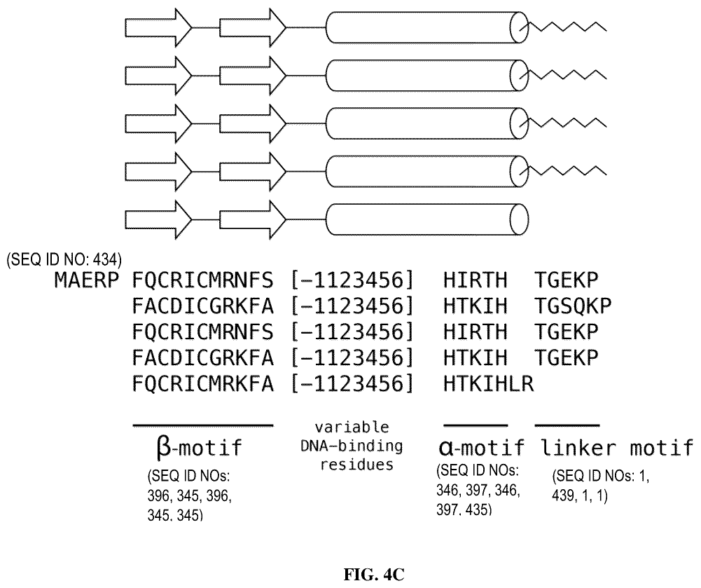

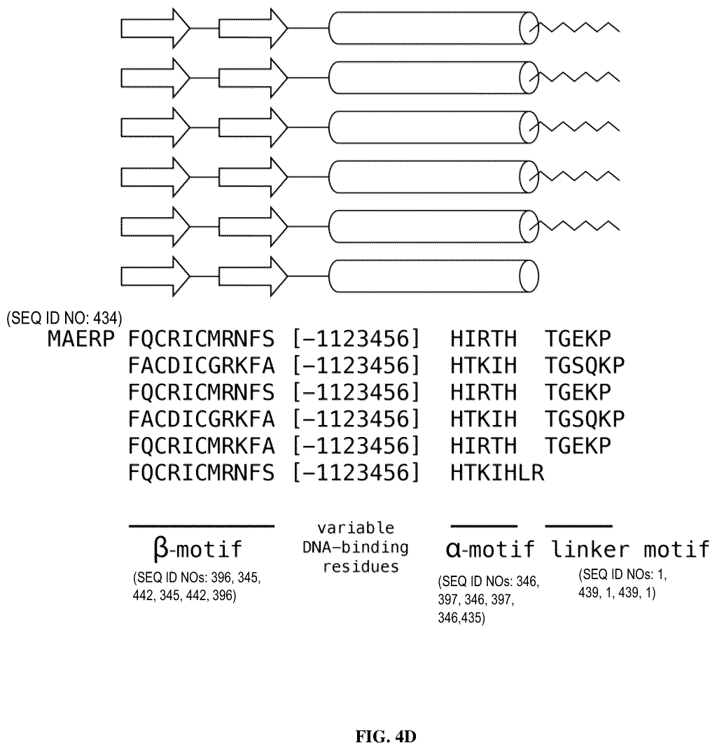

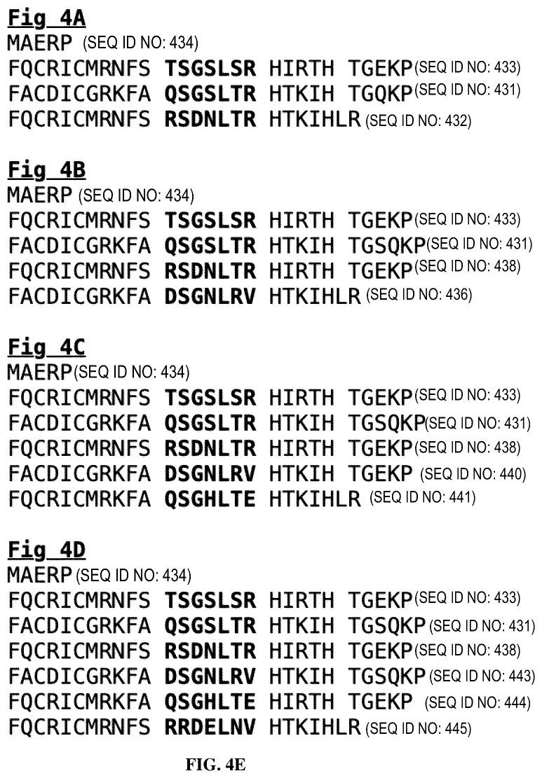

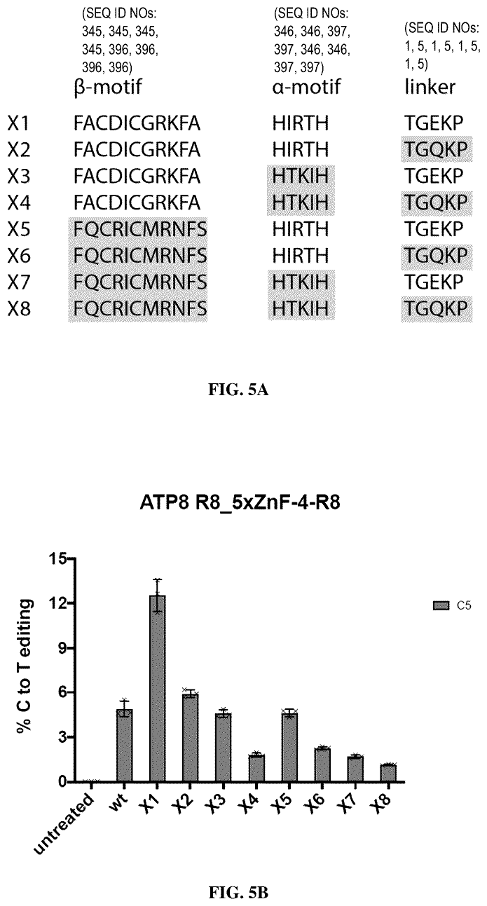

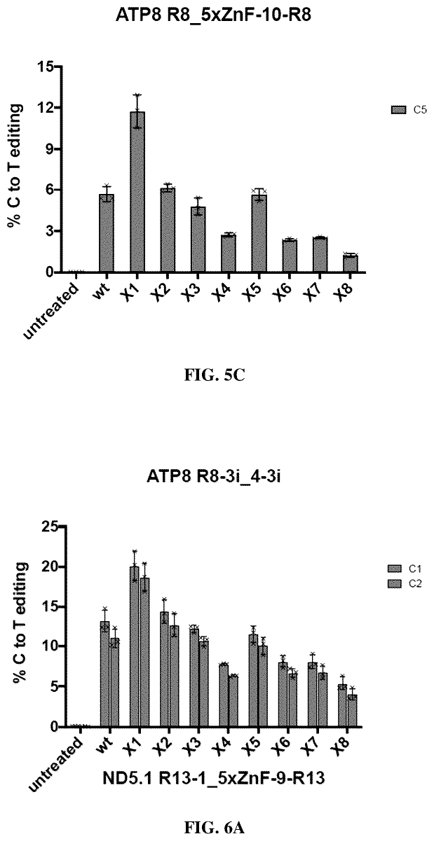

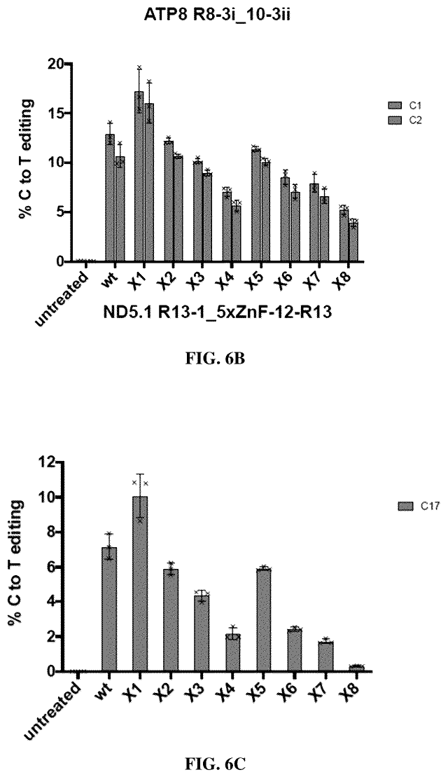

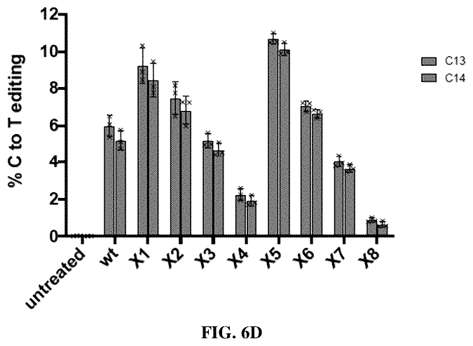

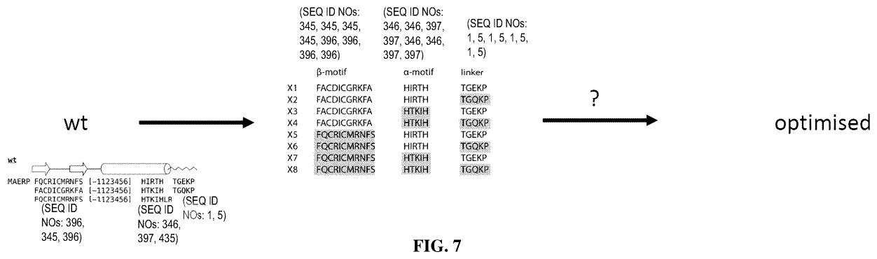

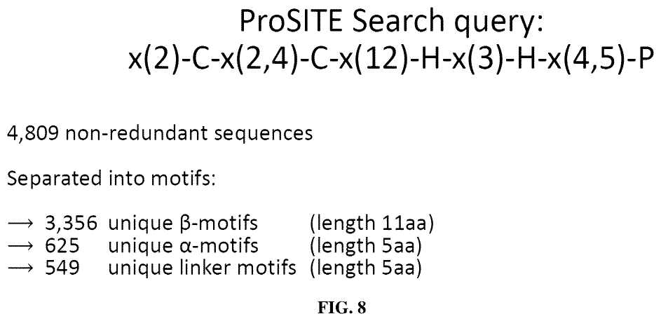

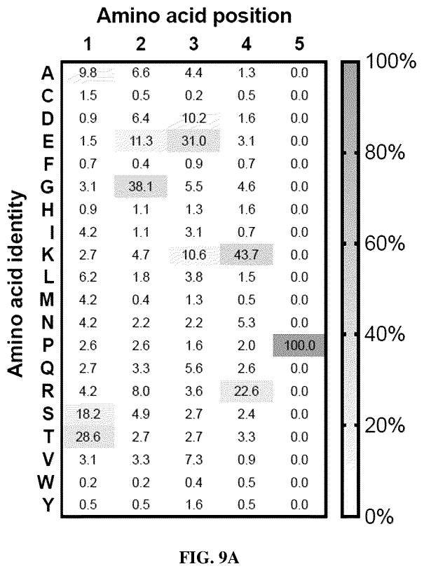

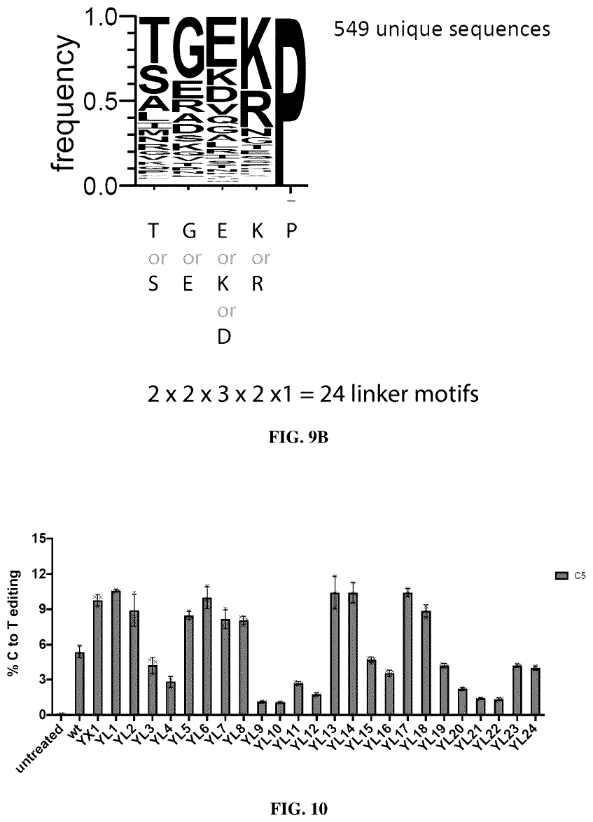

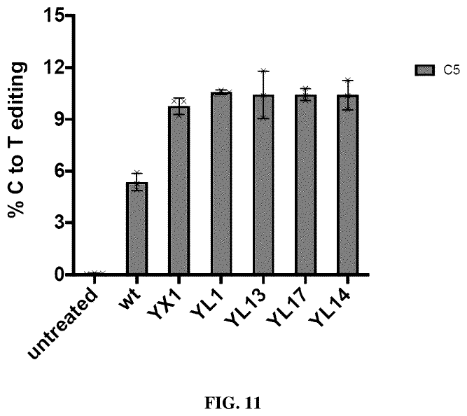

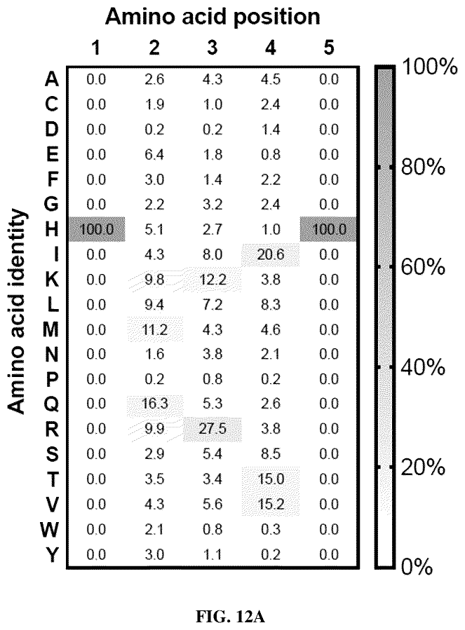

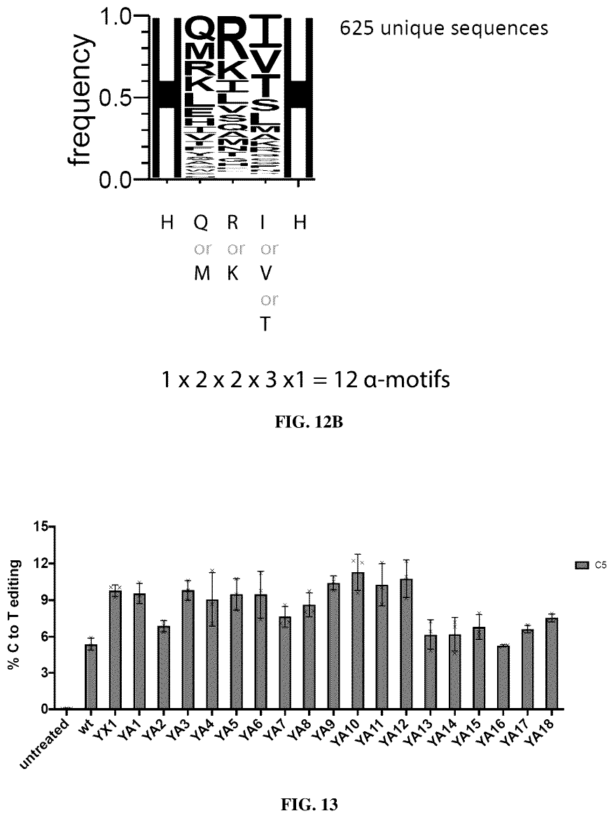

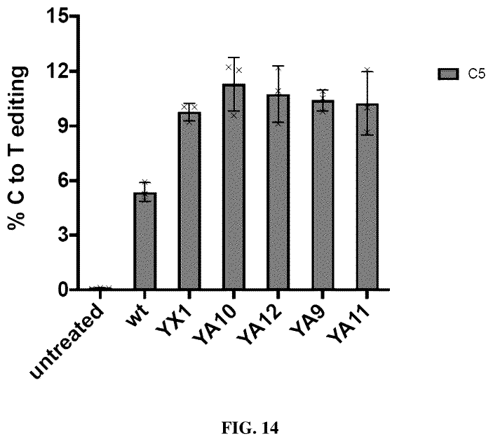

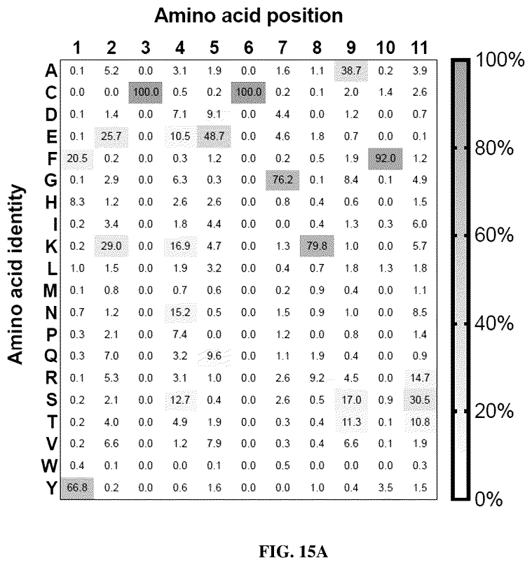

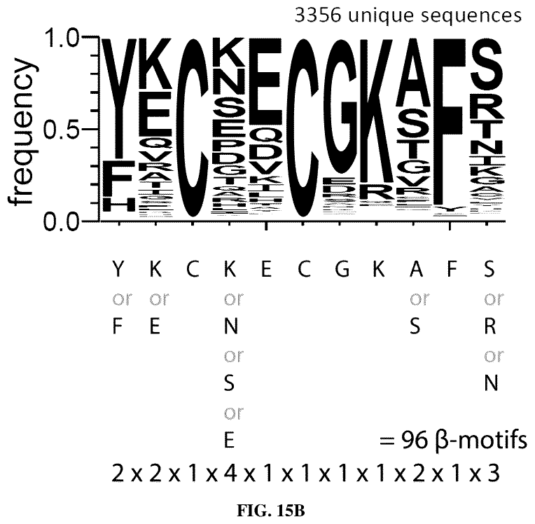

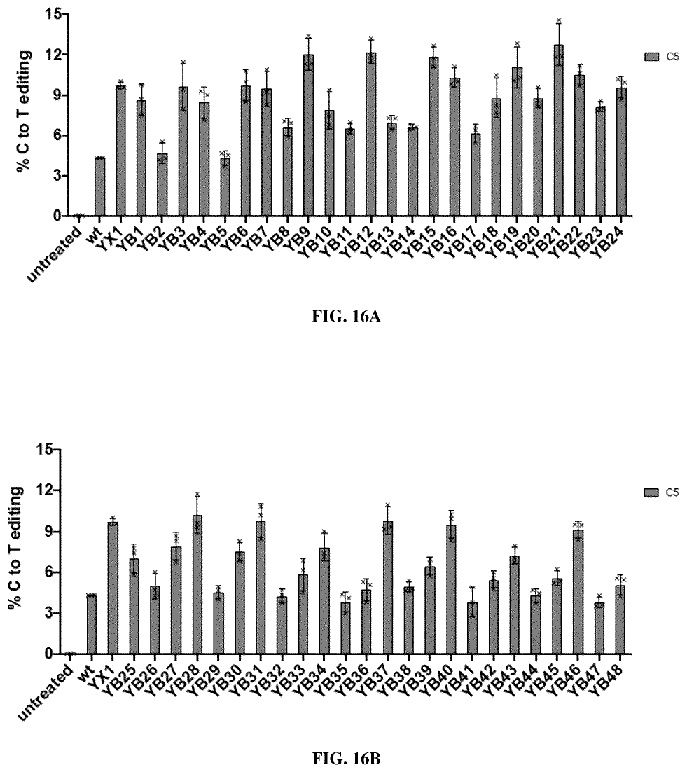

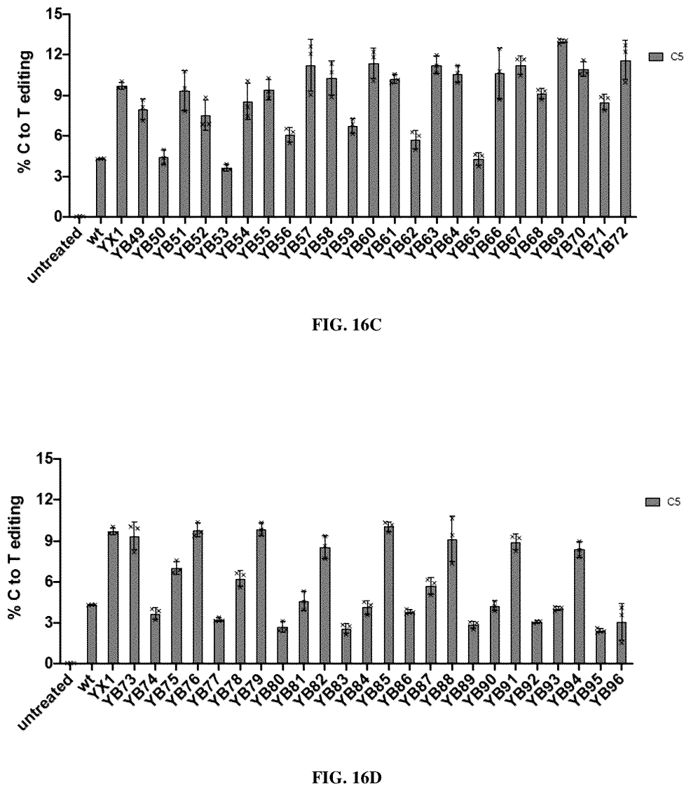

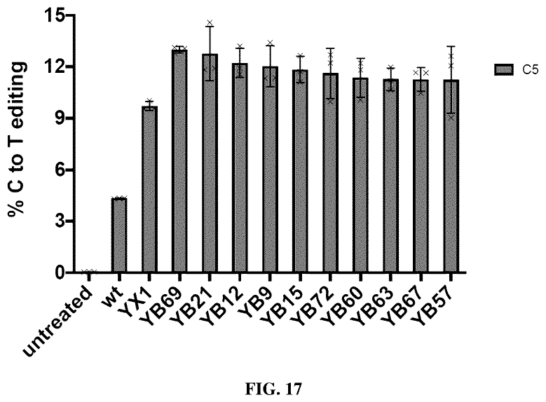

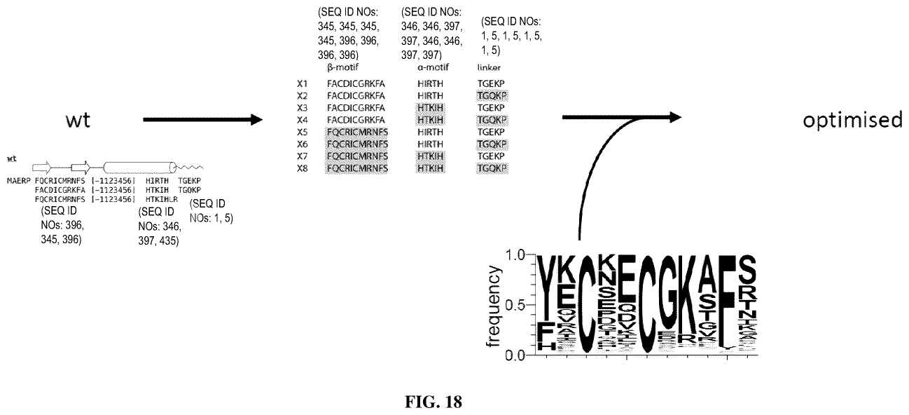

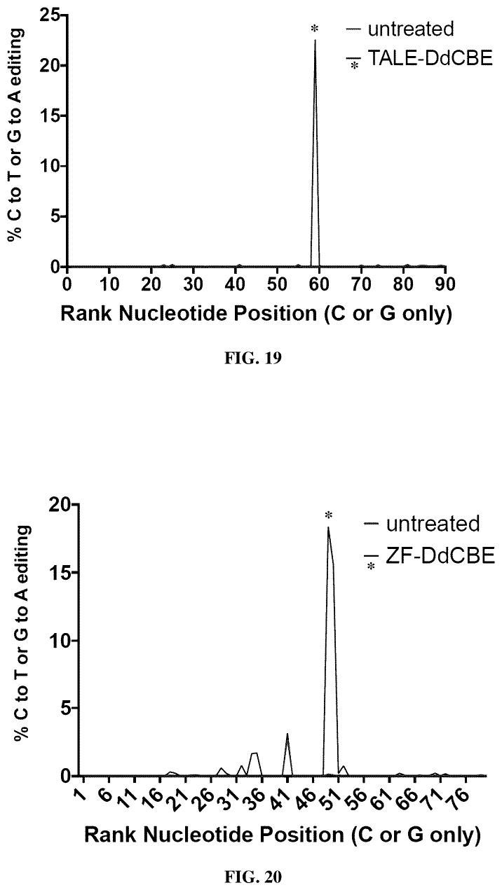

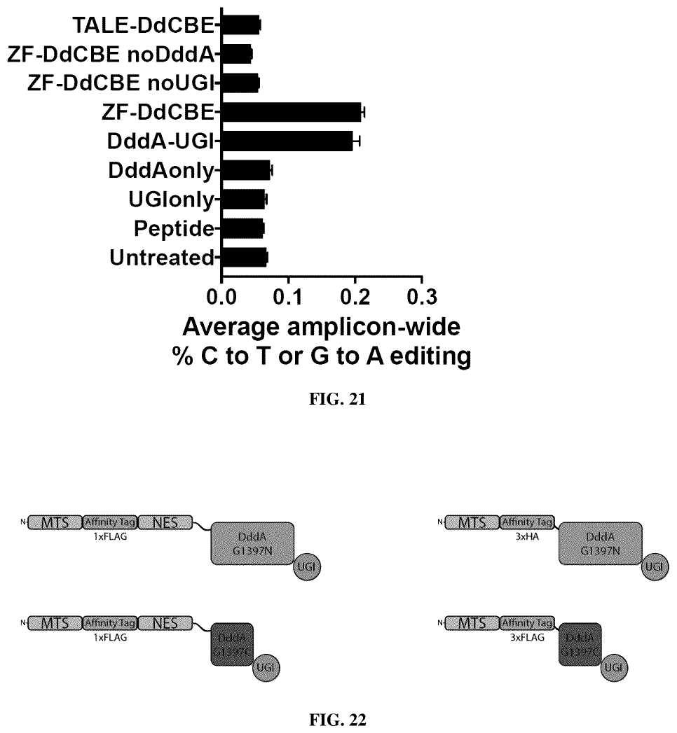

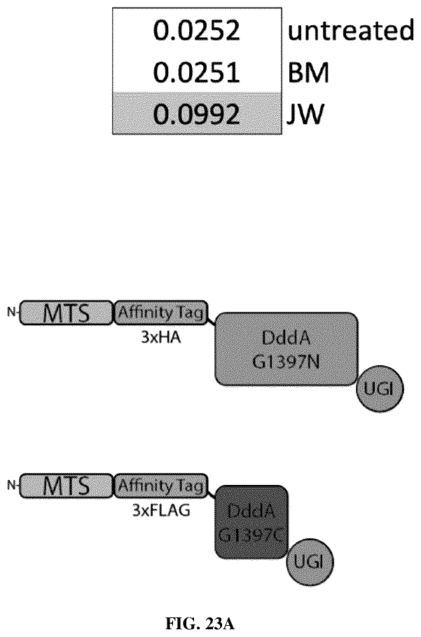

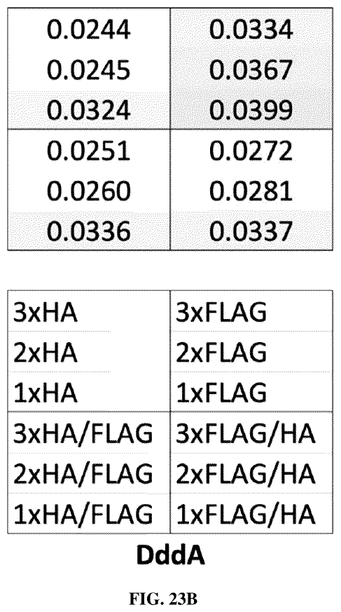

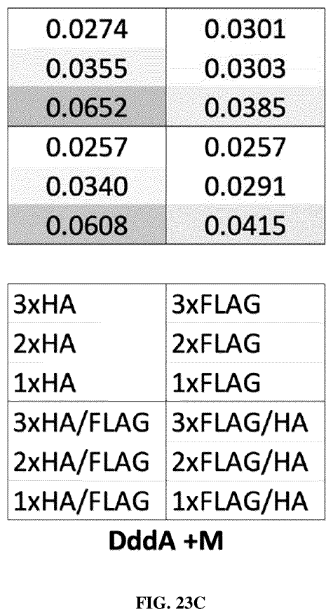

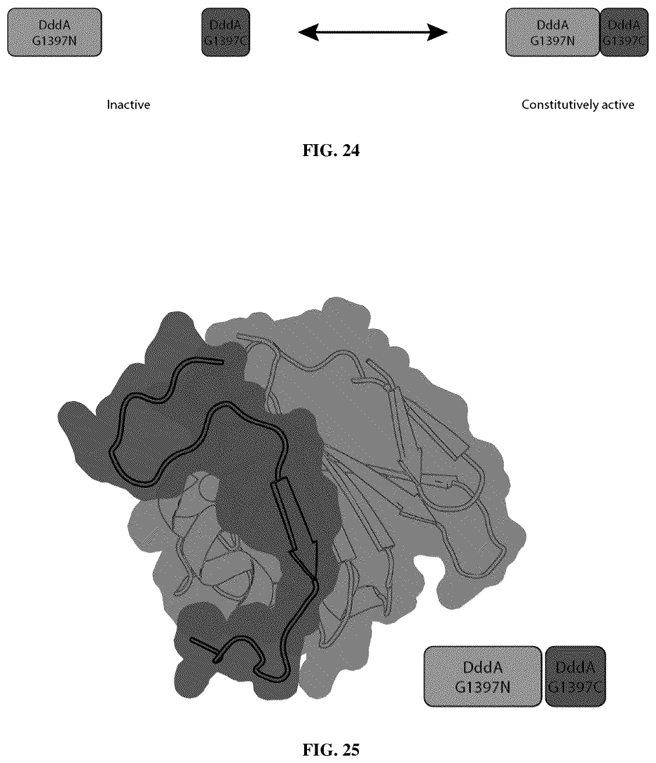

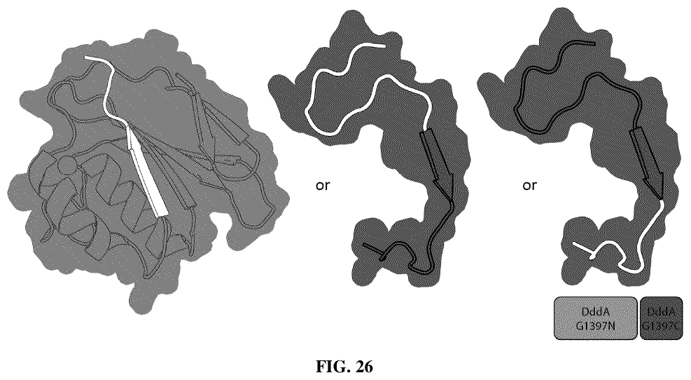

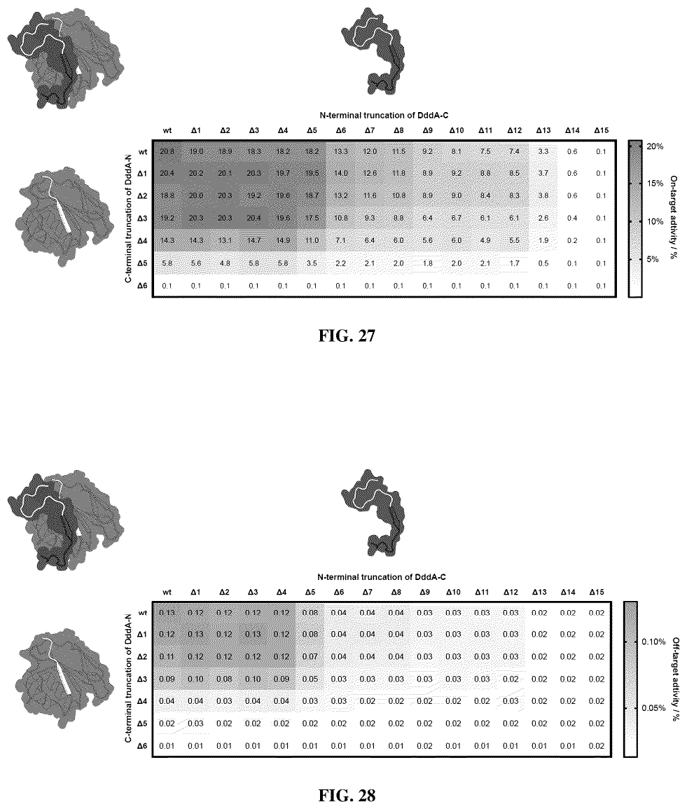

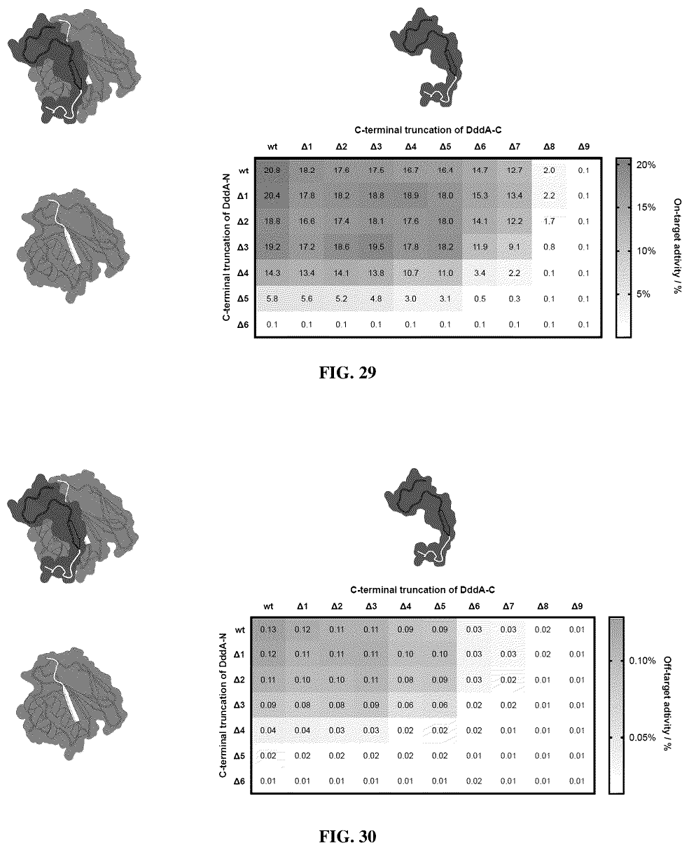

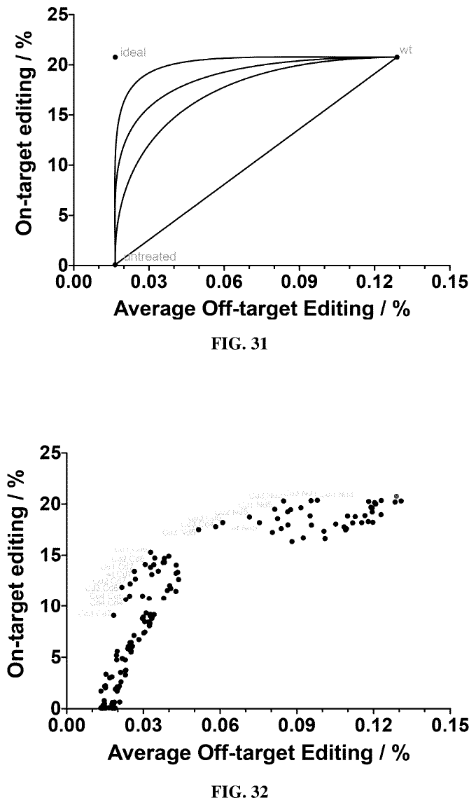

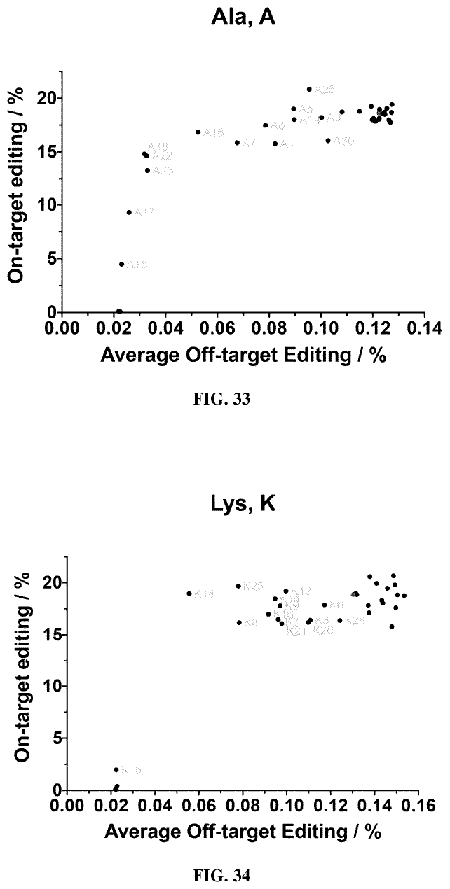

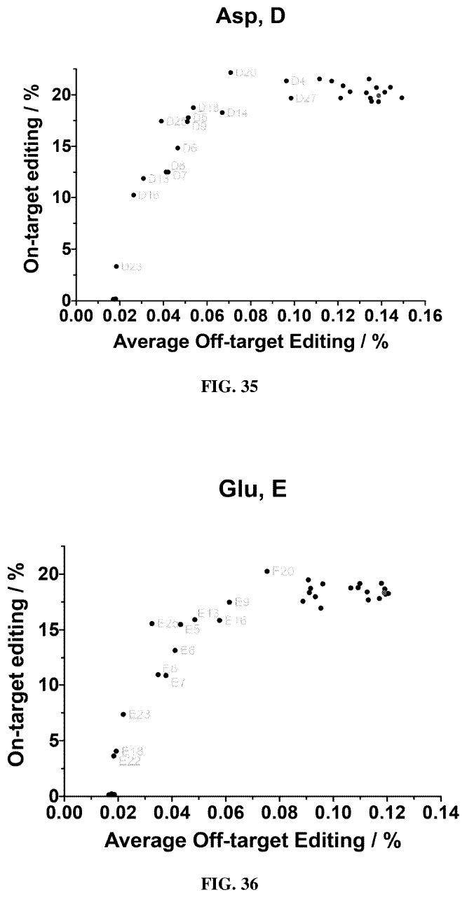

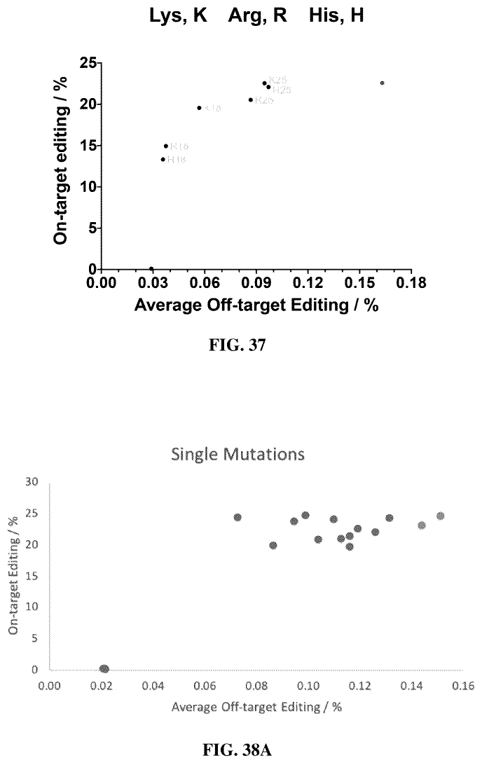

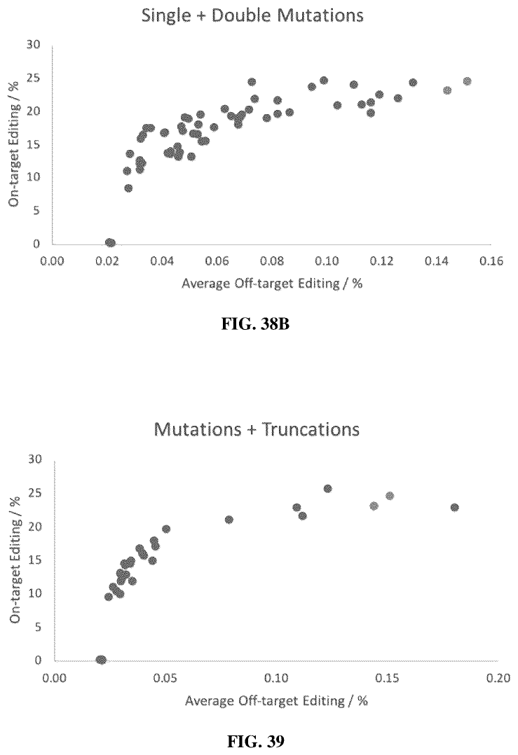

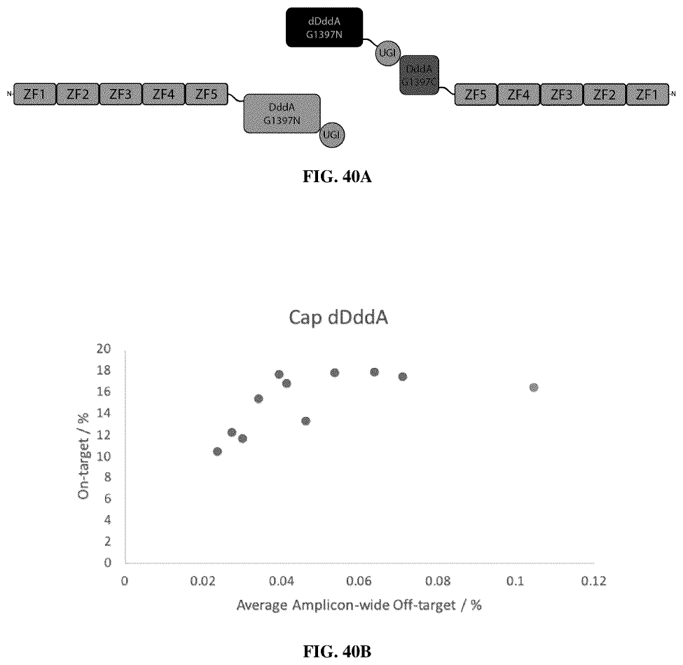

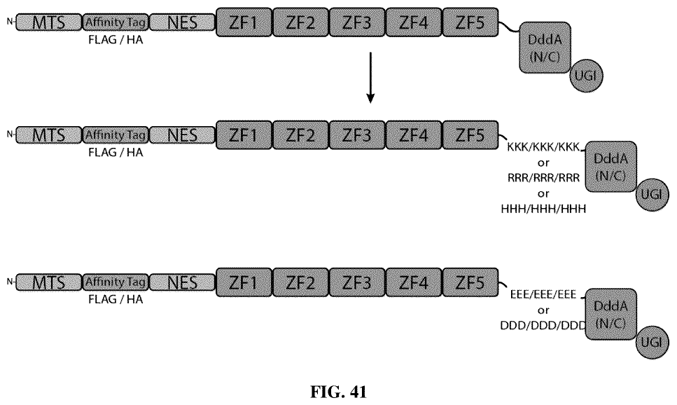

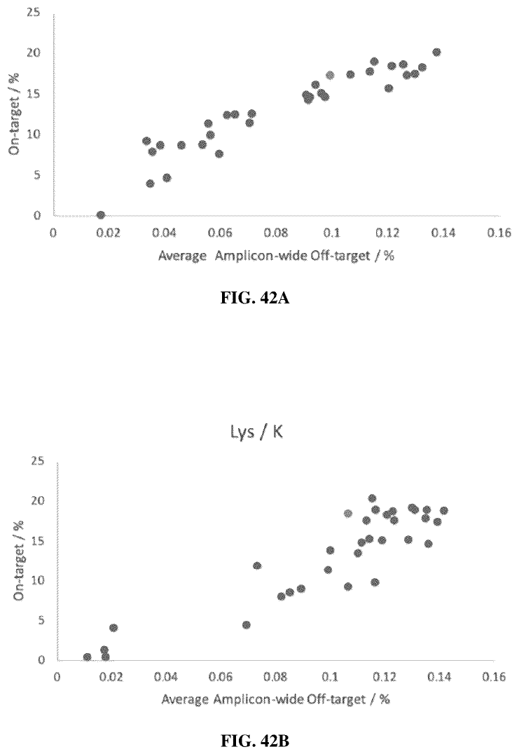

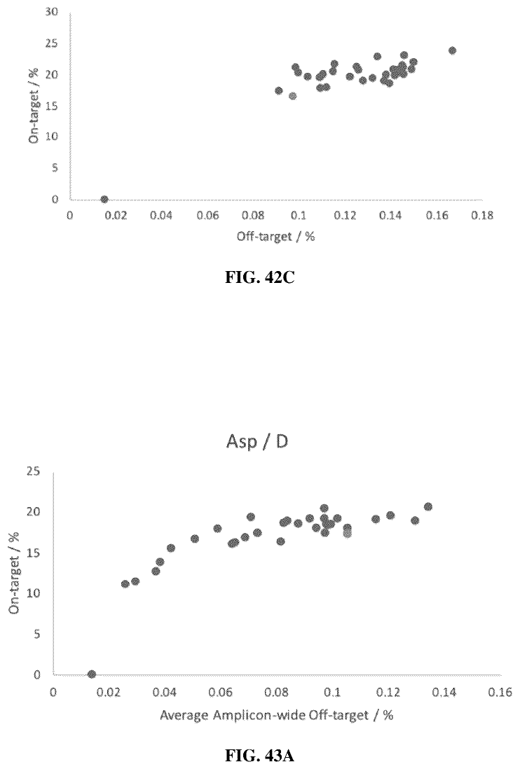

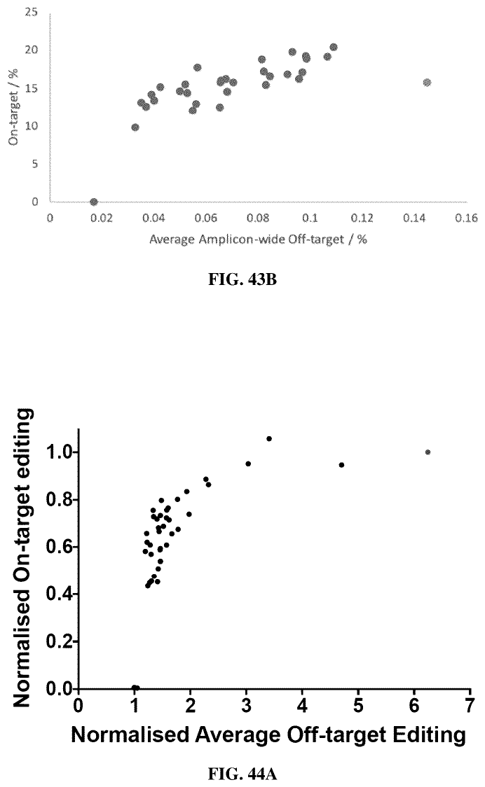

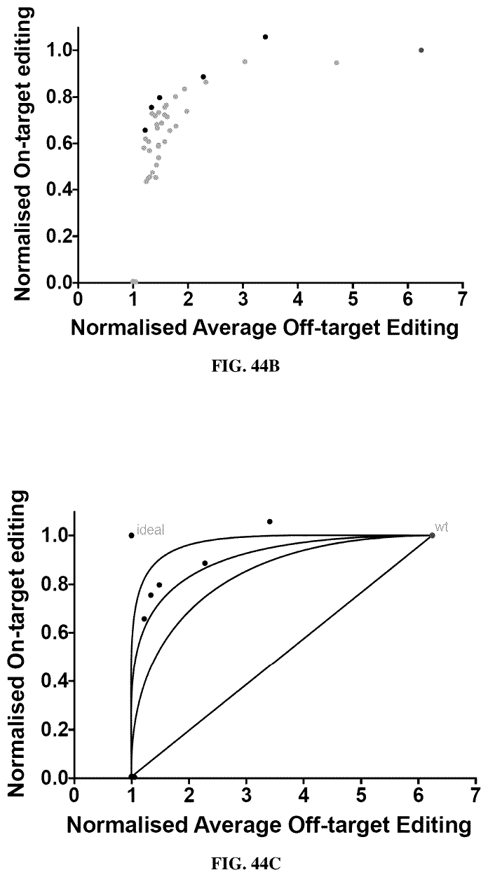

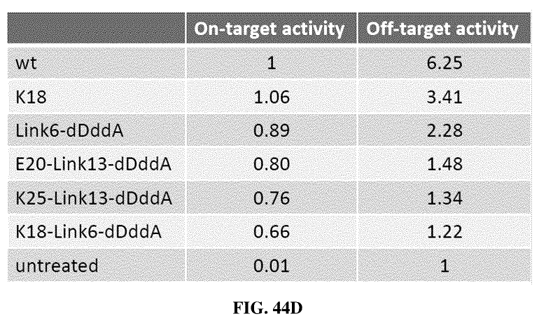

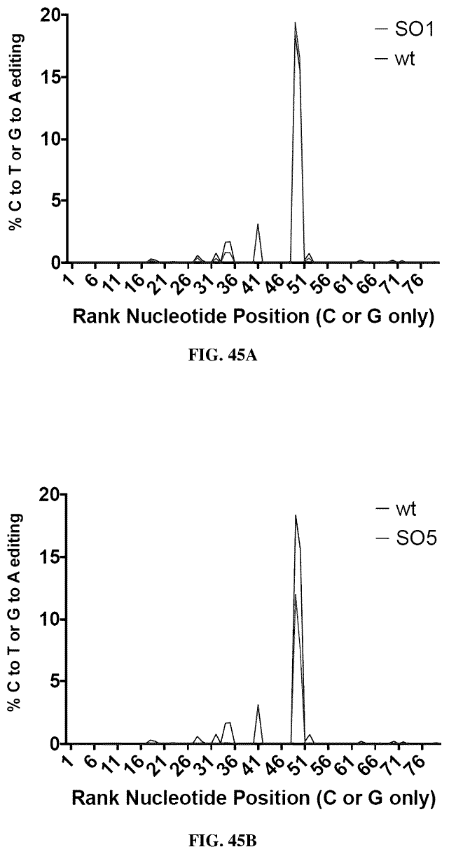

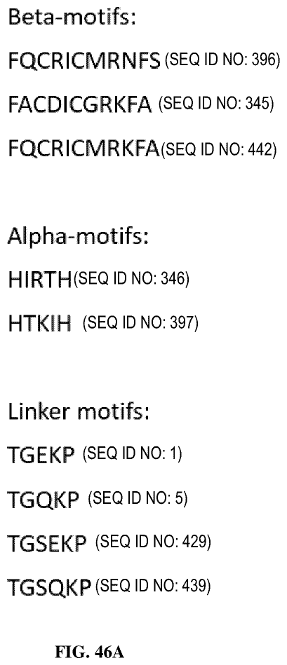

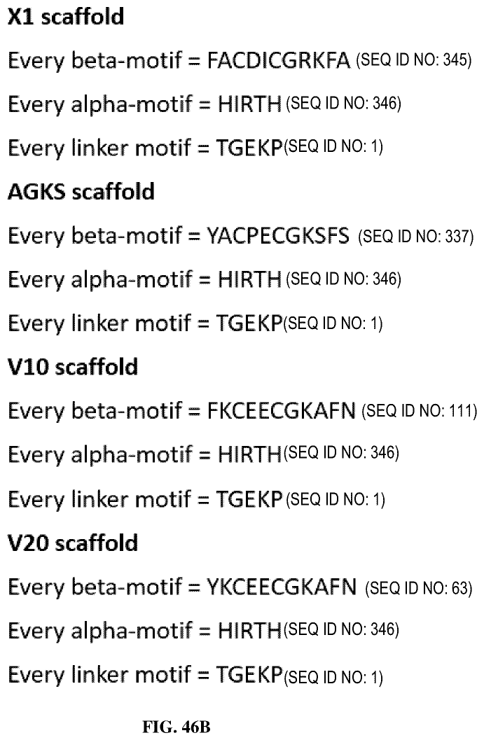

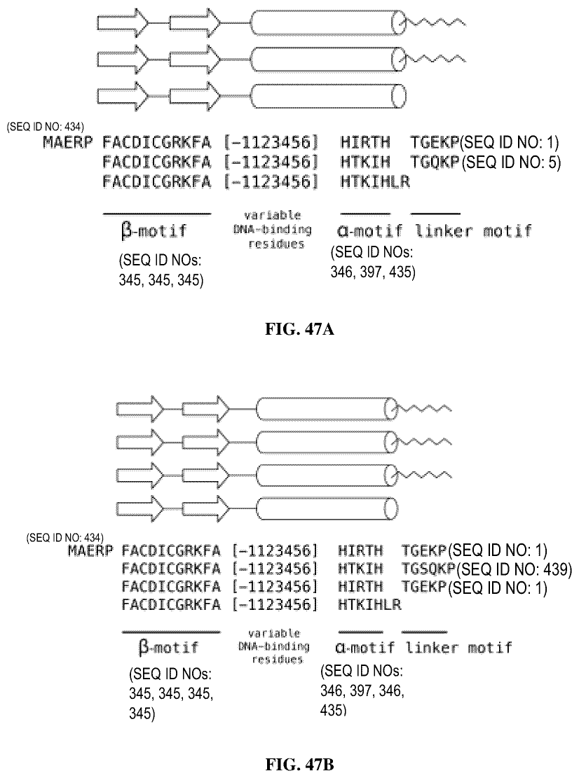

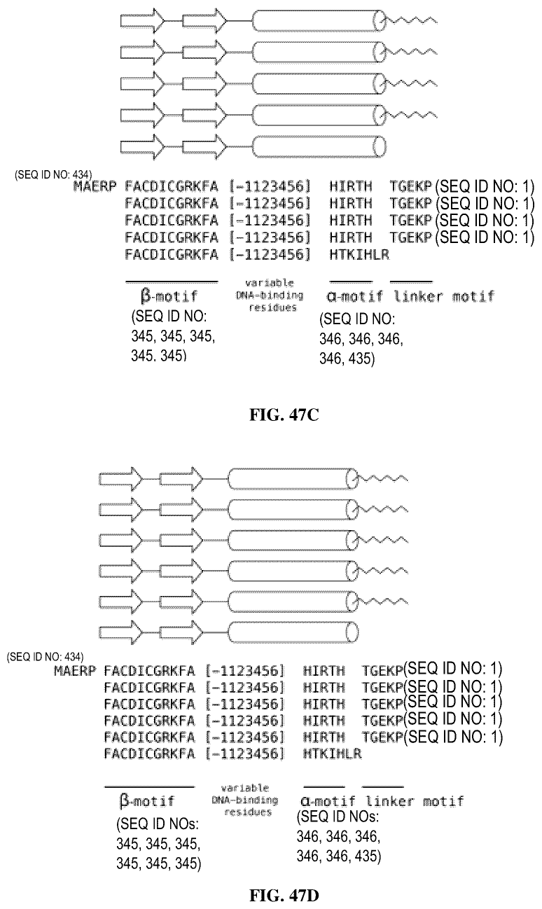

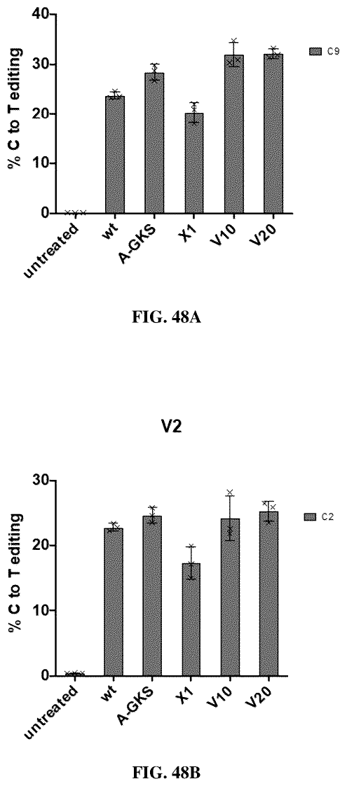

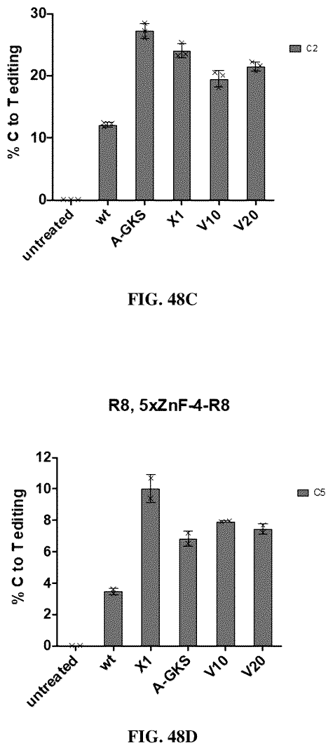

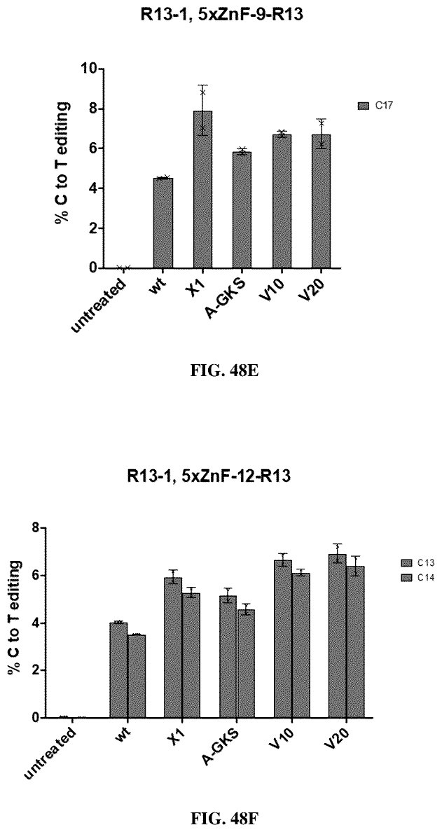

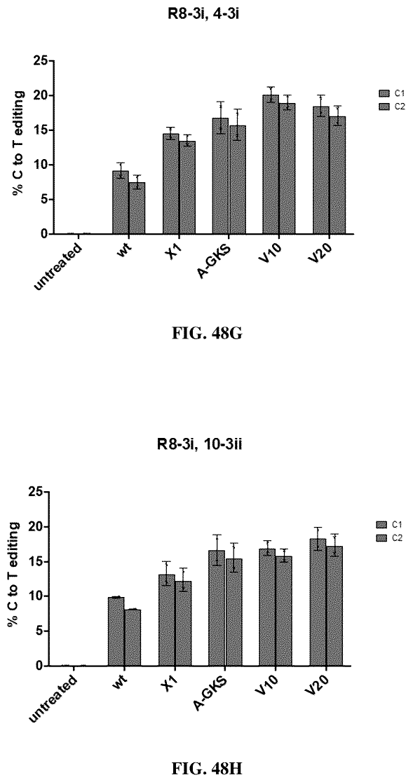

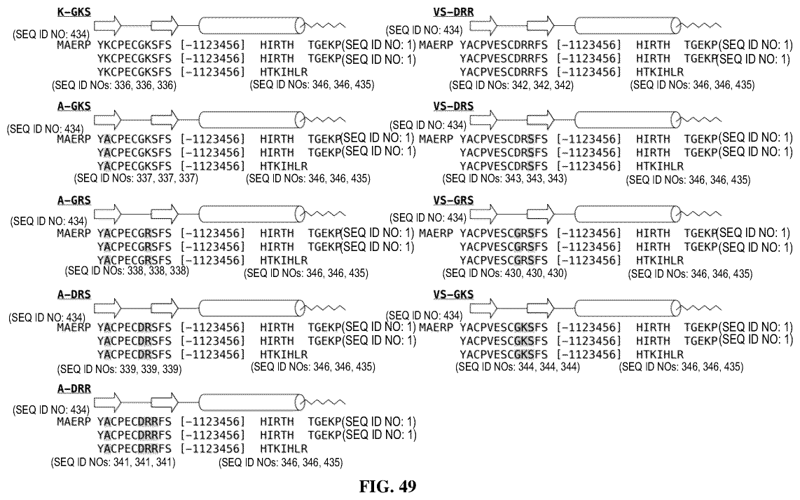

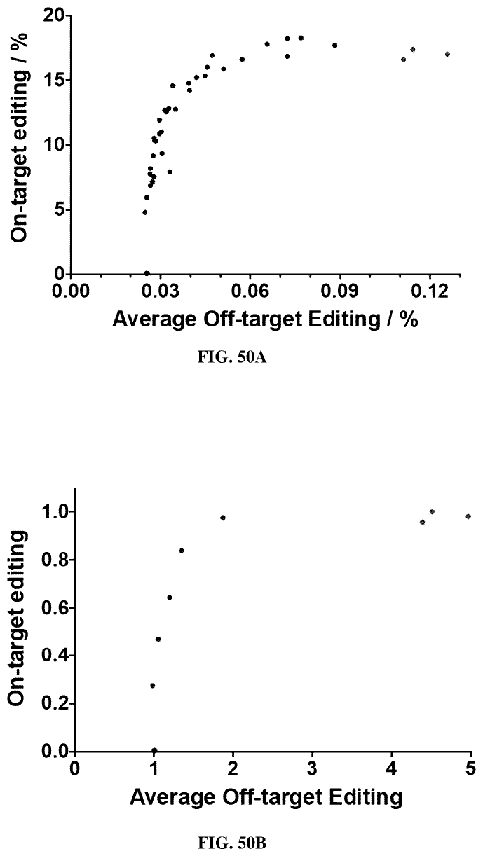

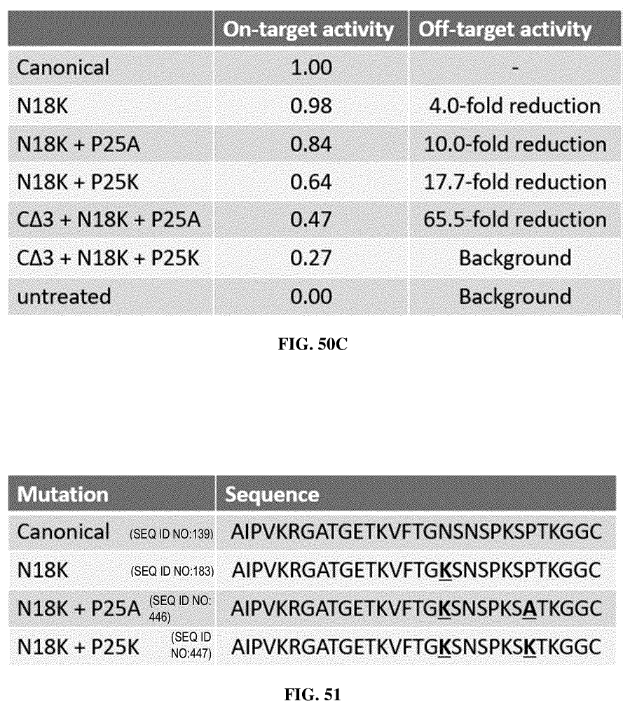

In some embodiments, the napDNAbp is an RNA-programmable nuclease, which, when in a complex with an RNA, may be referred to as a nuclease:RNA complex. Typically, the bound RNA(s) is referred to as a guide RNA (gRNA). gRNAs can exist as a complex of two or more RNAs, or as a single RNA molecule. gRNAs that exist as a single RNA molecule may be referred to as single-guide RNAs (sgRNAs), though “gRNA” is used interchangeably to refer to guide RNAs that exist as either single molecules or as a complex of two or more molecules. Typically, gRNAs that exist as single RNA species comprise two domains: (1) a domain that shares homology to a target nucleic acid (e.g., and directs binding of a Cas9 (or equivalent) complex to the target); and (2) a domain that binds a Cas9 protein. In some embodiments, domain (2) corresponds to a sequence known as a tracrRNA and comprises a stem-loop structure. For example, in some embodiments, domain (2) is homologous to a tracrRNA as depicted in FIG. 1E of Jinek et al., Science 337:816-821(2012), the entire contents of which is incorporated herein by reference. Other examples of gRNAs (e.g., those including domain 2) can be found in U.S. Pat. No. 9,340,799, entitled “mRNA-Sensing Switchable gRNAs,” and International Patent Application No. PCT/US2014/054247, filed Sep. 6, 2013, published as WO 2015/035136 and entitled “Delivery System For Functional Nucleases,” the entire contents of each of which are incorporated herein by reference. In some embodiments, a gRNA comprises two or more of domains (1) and (2) and may be referred to as an “extended gRNA.” For example, an extended gRNA will, e.g., bind two or more Cas9 proteins and bind a target nucleic acid at two or more distinct regions, as described herein. The gRNA comprises a nucleotide sequence that complements a target site, which mediates binding of the nuclease/RNA complex to said target site, providing the sequence specificity of the nuclease:RNA complex. In some embodiments, the RNA-programmable nuclease is the (CRISPR-associated system) Cas9 endonuclease, for example Cas9 (Csn1) from Streptococcus pyogenes (see, e.g., “Complete genome sequence of an M1 strain of Streptococcus pyogenes.” Ferretti J. J. et al., Proc. Natl. Acad. Sci. U.S.A. 98:4658-4663(2001); “CRISPR RNA maturation by trans-encoded small RNA and host factor Rnase III.” Deltcheva E. et al., Nature 471:602-607(2011); and “A programmable dual-RNA-guided DNA endonuclease in adaptive bacterial immunity.” Jinek M. et al., Science 337:816-821(2012), the entire contents of each of which are incorporated herein by reference.

Since the napDNAbp nucleases (e.g., Cas9) use RNA:DNA hybridization to target DNA cleavage sites, these proteins are able to be targeted, in principle, to any sequence specified by the guide RNA. Methods of using napDNAbp nucleases, such as Cas9, for site-specific cleavage (e.g., to modify a genome) are known in the art (see e.g., Cong, L. et al. Multiplex genome engineering using CRISPR/Cas systems. Science 339, 819-823 (2013); Mali, P. et al. RNA-guided human genome engineering via Cas9. Science 339, 823-826 (2013); Hwang, W. Y. et al. Efficient genome editing in zebrafish using a CRISPR-Cas system. Nature Biotechnology 31, 227-229 (2013); Jinek, M. et al. RNA-programmed genome editing in human cells. eLife 2, e00471 (2013); Dicarlo, J. E. et al., Genome engineering in Saccharomyces cerevisiae using CRISPR-Cas systems. Nucleic Acid Res. (2013); Jiang, W. et al. RNA-guided editing of bacterial genomes using CRISPR-Cas systems. Nature Biotechnology 31, 233-239 (2013); the entire contents of each of which are incorporated herein by reference).

### Nickase

The term “nickase” refers to a napDNAbp having only a single nuclease activity that cuts only one strand of a target DNA, rather than both strands. Thus, a nickase type napDNAbp does not leave a double-strand break. In some embodiments, any of the base editors disclosed herein may comprise a nickase (such as a Cas9 nickase) fused, for example, to any of the DddA variants disclosed herein.

### Nuclear Localization Signal

In various embodiments, the base editors or the polypeptides that comprise the base editors disclosed herein (e.g., the zinc finger domain-containing protein and DddA variant fusions described herein) may be further engineered to include one or more nuclear localization signals.

A nuclear localization signal or sequence (NLS) is an amino acid sequence that tags, designates, or otherwise marks a protein for import into the cell nucleus by nuclear transport. Typically, this signal consists of one or more short sequences of positively charged lysine or arginine residues exposed on the protein surface. Different nuclear localized proteins may share the same NLS. An NLS has the opposite function of a nuclear export signal (NES), which targets proteins out of the nucleus. Thus, a single nuclear localization signal can direct the entity with which it is associated to the nucleus of a cell. Such sequences may be of any size and composition, for example more than 4, 5, 6, 7, 8, 9, 10, 12, 15, 20, or 25 amino acids, but will preferably comprise at least a four to eight amino acid sequence known to function as a nuclear localization signal (NLS).

### Nucleic Acid Molecule

The term “nucleic acid,” as used herein, refers to a polymer of nucleotides. The polymer may include natural nucleosides (i.e., adenosine, thymidine, guanosine, cytidine, uridine, deoxyadenosine, deoxythymidine, deoxyguanosine, and deoxycytidine), nucleoside analogs (e.g., 2-aminoadenosine, 2-thiothymidine, inosine, pyrrolo-pyrimidine, 3-methyl adenosine, 5-methylcytidine, C5 bromouridine, C5 fluorouridine, C5 iodouridine, C5 propynyl uridine, C5 propynyl cytidine, C5 methylcytidine, 7 deazaadenosine, 7 deazaguanosine, 8 oxoadenosine, 8 oxoguanosine, 0(6) methylguanine, 4-acetylcytidine, 5-(carboxyhydroxymethyl)uridine, dihydrouridine, methylpseudouridine, 1-methyl adenosine, 1-methyl guanosine, N6-methyl adenosine, and 2-thiocytidine), chemically modified bases, biologically modified bases (e.g., methylated bases), intercalated bases, modified sugars (e.g., 2′-fluororibose, ribose, 2′-deoxyribose, 2′-O-methylcytidine, arabinose, and hexose), or modified phosphate groups (e.g., phosphorothioates and 5′-N-phosphoramidite linkages).

Programmable DNA Binding Protein (pDNAbp)

As used herein, the term “programmable DNA binding protein,” “pDNA binding protein,” “pDNA binding protein domain” or “pDNAbp” refers to any protein that localizes to and binds a specific target DNA nucleotide sequence (e.g., a gene locus of a genome). This term embraces RNA-programmable proteins, which associate (e.g., form a complex) with one or more nucleic acid molecules (i.e., which includes, for example, guide RNA in the case of Cas systems) that direct or otherwise program the protein to localize to a specific target nucleotide sequence (e.g., DNA sequence) that is complementary to the one or more nucleic acid molecules (or a portion or region thereof) associated with the protein. The term also embraces proteins which bind directly to a nucleotide sequence in an amino acid-programmable manner, e.g., zinc finger proteins and TALE proteins. Exemplary RNA-programmable proteins are CRISPR-Cas9 proteins, as well as Cas9 equivalents, homologs, orthologs, or paralogs, whether naturally occurring or non-naturally occurring (e.g., engineered or modified), and may include a Cas9 equivalent from any type of CRISPR system (e.g., type II, V, VI), including Cpf1 (a type-V CRISPR-Cas systems), C2c1 (a type V CRISPR-Cas system), C2c2 (a type VI CRISPR-Cas system), C2c3 (a type V CRISPR-Cas system), dCas9, GeoCas9, CjCas9, Cas12a, Cas12b, Cas12c, Cas12d, Cas12g, Cas12h, Cas12i, Cas13d, Cas14, Argonaute, and nCas9. Further Cas-equivalents are described in Makarova et al., “C2c2 is a single-component programmable RNA-guided RNA-targeting CRISPR effector,” Science 2016; 353(6299), the contents of which are incorporated herein by reference.

### Protein, Peptide, and Polypeptide

The terms “protein,” “peptide,” and “polypeptide” are used interchangeably herein and refer to a polymer of amino acid residues linked together by peptide (amide) bonds. The terms refer to a protein, peptide, or polypeptide of any size, structure, or function. Typically, a protein, peptide, or polypeptide will be at least three amino acids long. A protein, peptide, or polypeptide may refer to an individual protein or a collection of proteins. One or more of the amino acids in a protein, peptide, or polypeptide may be modified, for example, by the addition of a chemical entity such as a carbohydrate group, a hydroxyl group, a phosphate group, a farnesyl group, an isofarnesyl group, a fatty acid group, a linker for conjugation, functionalization, or other modification, etc. A protein, peptide, or polypeptide may also be a single molecule or may be a multi-molecular complex. A protein, peptide, or polypeptide may be just a fragment of a naturally occurring protein or peptide. A protein, peptide, or polypeptide may be naturally occurring, recombinant, or synthetic, or any combination thereof. Any of the proteins provided herein may be produced by any method known in the art. For example, the proteins provided herein may be produced via recombinant protein expression and purification, which is especially suited for fusion proteins comprising a peptide linker. Methods for recombinant protein expression and purification are well known, and include those described by Green and Sambrook, Molecular Cloning: A Laboratory Manual (4^{th} ed., Cold Spring Harbor Laboratory Press, Cold Spring Harbor, N.Y. (2012)), the contents of which are incorporated herein by reference.

### Split Site (e.g., of a DddA)

As used herein, the term “split site,” as in a split site of a DddA, refers to a specific peptide bond between any two immediately adjacent amino acid residues in the amino acid sequence of a DddA at which the complete DddA polypeptide is divided into two half portions, i.e., an N-terminal half portion and a C-terminal half portion. The N-terminal half portion of the DddA may be referred to as “DddA-N half” and the C-terminal half portion of the DddA may be referred to as the “DddA-C half.” Alternately, DddA-N half may be referred to as the “DddA-N fragment or portion” and the DddA-C half may be referred to as the “DddA-C fragment or portion.” Depending on the location of the split site, the DddA-N half and the DddA-C half may be the same or different size and/or sequence length. The term “half” does not connote the requirement that the DddA-N and DddA-C portions are identically half of the size and/or sequence length of a complete DddA, or that the split site is required to be at the midpoint of the complete DddA polypeptide. To the contrary, and as noted above, the split site can be between any pair of residues in the DddA polypeptide, thereby giving rise to half portions which are unequal in size and/or sequence length. For example, the split site may be such that the DddA polypeptide is split at amino acid position 1397 of DddA (e.g., as in the DddA variant proteins disclosed herein).

For clarity, as used herein, the term “half” when used in the context of a split molecule (e.g., protein, intein, delivery molecule, nucleic acid, etc.), shall not be interpreted to require, and shall not imply, that the size of the resulting portions (e.g., as “split” or broken into smaller portions) of the molecule are one-half (e.g., ½, 50%) of the original molecule. The term shall be interpreted to be illustrative of the idea that they are portion(s) of a larger molecule that has been broken into smaller fragments (e.g., portions), but that when reconstituted may regain the activity of the molecule as a whole. Thus, by way of example, a half (e.g., portion) may be any portion of the molecule from which it is obtained (e.g., is less than 100% of the whole of the molecule), such that there is at least one additional portion formed (e.g., a second half, other half, second portion), which also is less than 100% of the whole of the molecule. It is important to note that the molecule may be formed into additional portions (e.g., third, fourth, etc., halves (e.g., portions)), and such additional halves do not constitute a molecule larger than or in addition to the whole from which they were derived. Further, it should be noted that in the event there are more than two halves (e.g., two portions) formed from the splitting of a molecule, it may only require two of the portions to reconstitute the activity of the molecule as a whole. By way of example, if an enzyme is split into three halves (e.g., three portions), wherein the catalytic domain of the enzyme possessing the enzymatic activity of interest is only split into two halves (e.g., two portions), only the two portions of the catalytic domain may be necessary to be used to carry out the activity of interest. Thus, when referring to using two halves, it is not necessary that the two halves, together, comprise 100% of the whole of the molecule from which they were derived. In certain embodiments, the split site is within a loop region of the DddA.

As used herein, reference to “splitting a DddA at a split site” embraces direct and indirect means for obtaining two half portions of a DddA. In one embodiment, splitting a DddA refers to the direct splitting of a DddA polypeptide at a split site in the protein to obtain the DddA-N and DddA-C half portions. For example, the cleaving of a peptide bond between two adjacent amino acid residues at a split site may be achieved by enzymatic or chemical means. In another embodiment, a DddA may be split by engineering separate nucleic acid sequences, each encoding a different half portion of the DddA. Such methods can be used to obtain expression vectors for expressing the DddA half portions in a cell in order to reconstitute DddA activity.

### Subject

The term “subject,” as used herein, refers to an individual organism, for example, an individual mammal. In some embodiments, the subject is a human. In some embodiments, the subject is a non-human mammal. In some embodiments, the subject is a non-human primate. In some embodiments, the subject is a rodent. In some embodiments, the subject is a sheep, a goat, a cattle, a cat, or a dog. In some embodiments, the subject is a vertebrate, an amphibian, a reptile, a fish, an insect, a fly, or a nematode. In some embodiments, the subject is a research animal. In some embodiments, the subject is genetically engineered, e.g., a genetically engineered non-human subject. The subject may be of either sex and at any stage of development.

### Substitution

The terms “substitution” and “mutation,” as used herein, refer to a substitution of a residue within a sequence, e.g., a nucleic acid or amino acid sequence, with another residue; a deletion or insertion of one or more residues within a sequence; or a substitution of a residue within a sequence of a genome in a subject to be corrected. Mutations are typically described herein by identifying the original residue followed by the position of the residue within the sequence, and then by the identity of the newly substituted residue. Various methods for making the amino acid substitutions (mutations) provided herein are well known in the art, and are provided by, for example, Green and Sambrook, Molecular Cloning: A Laboratory Manual (4^{th} ed., Cold Spring Harbor Laboratory Press, Cold Spring Harbor, N.Y. (2012)). The terms mutation and substitution can include a variety of categories, such as single base polymorphisms, microduplication regions, indels, and inversions, and are not meant to be limiting in any way. Mutations can include “loss-of-function” mutations, which are mutations that reduce or abolish a protein activity. Most loss-of-function mutations are recessive, because in a heterozygote the second chromosome copy carries an unmutated version of the gene coding for a fully functional protein whose presence compensates for the effect of the mutation. There are some exceptions where a loss-of-function mutation is dominant, one example being haploinsufficiency, where the organism is unable to tolerate the approximately 50% reduction in protein activity suffered by the heterozygote. This is the explanation for a few genetic diseases in humans, including Marfan syndrome, which results from a mutation in the gene for the connective tissue protein called fibrillin. Mutations also embrace “gain-of-function” mutations, which are substitutions that confer an abnormal activity on a protein or cell that is otherwise not present in a normal (wild type) condition. Many gain-of-function mutations are in regulatory sequences rather than in coding regions, and they can therefore have a number of consequences. For example, a mutation might lead to one or more genes being expressed in the wrong tissues, these tissues gaining functions that they normally lack. Alternatively, the mutation could lead to overexpression of one or more genes involved in control of the cell cycle, thus leading to uncontrolled cell division and hence to cancer. Because of their nature, gain-of-function mutations are usually dominant.

### Target Site

The term “target site” refers to a sequence within a nucleic acid molecule that is edited by a zinc finger base editor disclosed herein. The target site further refers to the sequence within a nucleic acid molecule to which a base editor binds.

### Treatment

The terms “treatment,” “treat,” and “treating,” refer to a clinical intervention aimed to reverse, alleviate, delay the onset of, or inhibit the progress of a disease or disorder, or one or more symptoms thereof, as described herein. As used herein, the terms “treatment,” “treat,” and “treating” refer to a clinical intervention aimed to reverse, alleviate, delay the onset of, or inhibit the progress of a disease or disorder, or one or more symptoms thereof, as described herein. In some embodiments, treatment may be administered after one or more symptoms have developed and/or after a disease has been diagnosed. In other embodiments, treatment may be administered in the absence of symptoms, e.g., to prevent or delay onset of a symptom or inhibit onset or progression of a disease. For example, treatment may be administered to a susceptible individual prior to the onset of symptoms (e.g., in light of a history of symptoms and/or in light of genetic or other susceptibility factors). Treatment may also be continued after symptoms have resolved, for example, to prevent or delay their recurrence.

### Uracil Glycosylase Inhibitor (UGI)

The term “uracil glycosylase inhibitor” or “UGI,” as used herein, refers to a protein that is capable of inhibiting a uracil-DNA glycosylase base-excision repair enzyme. In some embodiments, a UGI domain comprises a wild-type UGI or a UGI as set forth in SEQ ID NO: 351. In some embodiments, the UGI proteins provided herein include fragments of UGI and proteins homologous to a UGI or a UGI fragment. For example, in some embodiments, a UGI domain comprises a fragment of the amino acid sequence set forth in SEQ ID NO: 351. In some embodiments, a UGI fragment comprises an amino acid sequence that comprises at least 60%, at least 65%, at least 70%, at least 75%, at least 80%, at least 85%, at least 90%, at least 95%, at least 96%, at least 97%, at least 98%, at least 99%, or at least 99.5% of the amino acid sequence as set forth in SEQ ID NO: 351. In some embodiments, a UGI comprises an amino acid sequence homologous to the amino acid sequence set forth in SEQ ID NO: 351, or an amino acid sequence homologous to a fragment of the amino acid sequence set forth in SEQ ID NO: 351. In some embodiments, proteins comprising UGI, or fragments of UGI or homologs of UGI, are referred to as “UGI variants.” A UGI variant shares homology to UGI, or a fragment thereof. For example, a UGI variant is at least 70% identical, at least 75% identical, at least 80% identical, at least 85% identical, at least 90% identical, at least 95% identical, at least 96% identical, at least 97% identical, at least 98% identical, at least 99% identical, at least 99.5% identical, or at least 99.9% identical to a wild type UGI or a UGI as set forth in SEQ ID NO: 351. In some embodiments, the UGI variant comprises a fragment of UGI, such that the fragment is at least 70% identical, at least 80% identical, at least 90% identical, at least 95% identical, at least 96% identical, at least 97% identical, at least 98% identical, at least 99% identical, at least 99.5% identical, or at least 99.9% to the corresponding fragment of wild-type UGI or a UGI as set forth in SEQ ID NO: 351. In some embodiments, the UGI comprises the following amino acid sequence: MTNLSDIIEKETGKQLVIQESILMLPEEVEEVIGNKPESDILVHTAYDESTDENVMLLTSD APEYKPWALVIQDSNGENKIKML (SEQ ID NO: 351) (P14739|UNGI_BPPB2 Uracil-DNA glycosylase inhibitor), or the same sequence but without the N-terminal methionine.

Other UGI proteins may include those described in Example 6, as follows:

| | | SEQ |
|---|---|---|
| | | ID |
| UGI | Sequence | NO: |
| Canonical | TNLSDIIEKETGKQLVIQESILMLPEEVEEVIGNK | 358 |
| UGI | PESDILVHTAYDESTDENVMLLTSDAPEYKPWALV | |
| | IQDSNGENKIKML | |
| UGI2 | MTLELQLKHYITNLFNLPKDEKWHCESIEEIADDI | 352 |
| | LPDQYVRLGALSNKILQTYTYYSDTLHESNIYPFI | |
| | LYYQKQLIAIGYIDENHDMDFLYLHNTIMPLLDQR | |
| | YLLTGGQ | |
| UGI3 | MNKNFDEVKADLRTVTGKKIEFKERLKNILRVQMN | 353 |
| | QLGFEDSYMIQVQVSSDQEEWVECHENMSLSDFEV | |
| | MYGNISGEIKRMTVVKYEEANIEKLVELKFEYEYA | |
| | KAHQEYIRAYTKLMSNTLYGRKPSL | |
| UGI5 | MNEEKMHYRDAIKEVELTMMSLDSHFRTHKEFTDS | 354 |
| | YLLVLILEDVVGETRVEVSEGLTFDEASYIIGGTS | |
| | DNILNMHMINYCEKNREEIYKWLKVSRVNTFKSNY | |
| | AKMLLNTAYGKDLLKGVVK | |
| UGI7 | MNNHFMSIGRNCSKCNNVRLNEDFSKSEEICNECF | 355 |
| | DKEERFVDSYTLIYITEDETGKRFEAILENQTIEE | |
| | TEIIYGNIIDKIIVWNVILTM | |
| UGI12 | DGNEHWEVHPGLSLSDFEVVYGNNPHQIVKLRLDK | 350 |
| | EVGGSGGSMVQNDFIDSYTLCWLLRDDSGGGGSMV | |
| | QNDFIDSYTLCWLLRDDDGNEHWEVHPGLSLSDFE | |
| | VVYGNNPHQIVKLRLDKEV | |

### Variant

As used herein, the term “variant” should be taken to mean the exhibition of qualities that have a pattern that deviates from what occurs in nature, e.g., a variant zinc finger protein is a zinc finger protein comprising one or more changes in amino acid residues as compared to a wild type zinc finger protein amino acid sequence. A variant deaminase is a deaminase comprising one or more changes in amino acid residues as compared to a wild type deaminase amino acid sequence. The term “variant” encompasses homologous proteins having at least 75%, at least 80%, at least 85%, at least 90%, at least 95%, or at least 99% identity with a reference sequence and having the same or substantially the same functional activity or activities as the reference sequence. The term also encompasses mutants, truncations, or domains of a reference sequence that display the same or substantially the same functional activity or activities as the reference sequence.

### Vector

The term “vector,” as used herein, refers to a nucleic acid that can be modified to encode a gene of interest and that is able to enter a host cell, mutate, and replicate within the host cell, and then transfer a replicated form of the vector into another host cell. Exemplary suitable vectors include viral vectors, such as retroviral vectors or bacteriophages and filamentous phage, and conjugative plasmids. Additional suitable vectors will be apparent to those of skill in the art based on the instant disclosure.

### Wild Type

As used herein the term “wild type” is a term of the art understood by skilled persons and means the typical form of an organism, strain, gene, or characteristic as it occurs in nature as distinguished from mutant or variant forms.

### Zinc Finger DNA Binding Protein and Zinc Finger Motifs

A “zinc finger DNA binding protein or polypeptide” is a protein or polypeptide that comprises at least one zinc finger motif and is capable of and/or has the property of being able to bind to a DNA molecule in a “programmable manner.” As used herein, a “zinc finger motif” is a polypeptide comprising an amino acid sequence that folds into a three-dimensional structure that is held together and stabilized by the coordinated binding by certain amino acid residues (e.g., cysteine and histidine) in the zinc finger motif to a zinc ion. The amino acid sequence of the zinc finger motif “programs” or determines the sequence of DNA to which it can bind. As used herein, a protein domain that comprises at least one zinc finger motif may be referred to as a “zinc finger domain.” Further, a zinc finger DNA binding protein may be regarded more broadly as a type of “zinc finger domain-containing protein or polypeptide.” A zinc finger domain-containing protein or polypeptide is any protein or polypeptide that comprises at least one zinc finger motif. In certain embodiments, the zinc finger domain-containing protein may comprise an array of two or more zinc finger motifs arranged in a continuous or non-continuous pattern or repeating array (e.g., 2, 3, 4, 5, 6, 7, 8, 9, 10, 11, or 12 or more zinc finger motifs).

Zinc finger DNA binding proteins or polypeptides) (which may be referred more generally as “zinc finger protein or polypeptide” or “ZFP”) can be “engineered” to bind to a predetermined or target nucleotide sequence. Non-limiting examples of methods for engineering zinc finger proteins include sequence design and selection approaches. Such engineered proteins do not occur in nature. Rational criteria for engineering such proteins include application of substitution rules and computerized algorithms for processing information in a database storing information of existing ZFP designs, sequences, and binding data. See, for example, U.S. Pat. Nos. 6,140,081; 6,453,242; 6,534,261; and 6,785,613; see, also WO 98/53058; WO 98/53059; WO 98/53060; WO 02/016536; and WO 03/016496; and U.S. Pat. Nos. 6,746,838; 6,866,997; and 7,030,215, each of which are incorporated herein by reference.

The present application also relates to zinc finger nucleases (“ZFNs”). Zinc finger nucleases (“ZFNs”) are artificial restriction enzymes generated by fusing a zinc finger DNA-binding protein or domain to a DNA-cleavage domain. Zinc finger DNA-binding domains can be engineered to target specific desired DNA sequences, and this enables zinc finger nucleases to target unique sequences within complex genomes.

The DNA-binding domains of individual ZFNs typically contain between three and six individual zinc finger motifs (each containing a β-motif, a DNA recognition motif, and an α-motif as described further herein) and can each recognize between 9 and 18 base pairs. The repeating units of individual zinc finger motifs of the DNA-binding domain can be referred to as a “zinc finger repeat” or “zinc finger array.” Each individual zinc finger motif is typically joined together by a linker motif. If the zinc finger domains are specific for their intended target site, a pair of 3-finger ZFNs that recognize a total of 18 base pairs can, in theory, target a single locus in a mammalian genome. The most straightforward method to generate new zinc finger arrays is to combine smaller zinc finger “modules” of known specificity. The most common modular assembly process involves combining three separate zinc finger motifs that can each recognize a 3 base pair DNA sequence to generate a 3-finger zinc finger array that can recognize a 9 base pair target site.

### DETAILED DESCRIPTION

The present disclosure is based on the development by the inventors of engineered zinc finger domain-containing proteins, DddA variants, and fusion proteins comprising the same that display increased on-target base editing activity and/or decreased off-target base editing activity. In particular, the proteins and fusion proteins provided herein may be especially useful for editing mitochondrial DNA due to the small size of zinc finger proteins, as described further herein. Thus, the present disclosure provides zinc finger domain-containing proteins comprising optimized α-, β-, and/or linker motifs, and fusion proteins comprising said zinc finger domain-containing proteins fused to an effector domain (e.g., a deaminase, or any other effector protein including but not limited to those described herein). The present disclosure also provides DddA variants and fusion proteins comprising said DddA variants (for example, fused to a programmable DNA binding protein, such as any of the zinc finger domain-containing proteins disclosed herein, or a CRISPR/Cas9 protein). Methods for editing DNA (including, e.g., genomic DNA and mitochondrial DNA) using the fusion proteins described herein are also provided by the present disclosure. The present disclosure further provides polynucleotides, vectors, cells, kits, and pharmaceutical compositions comprising the zinc finger domain-containing proteins, DddA variants, and fusion proteins described herein.

### Zinc Finger Domain-Containing Proteins

In one aspect, the present disclosure provides engineered zinc finger domain-containing proteins. Engineered zinc finger arrays are most commonly constructed based on the sequence of Zif268, a murine transcription factor. As described further herein, it was found by the inventors that zinc finger scaffold sequences with improved activity (for example, improved base editing activity when linked to a fusion protein in the context of a deaminase) could be developed by searching the human proteome for the ZF consensus sequence: x(2)-C-x(2,4)-C-x(12)-H-x(3)-H-x(4,5)-P, where C and H are conserved Cys and His residues that coordinate the Zn²⁺ ion, P is a conserved Pro residue at the end of the linker motif, and x can be any amino acid residue. Through this search, several ZF sequences from the human proteome were discovered, and these sequences were separated and filtered to extract new beta-motif sequences, new alpha-motif sequences, and new linker motif sequences. As described herein, all of the sequences identified within each class were aligned, and an amino acid frequency calculation was performed to determine the frequency at which each amino acid was found at each position within each of the three types of motif sequences. This provided a basis set of amino acids from which to construct new motif sequences. All possible permutations of these sequences were created, which resulted in the creation of new linker motifs, alpha-motifs, and beta-motifs. Sequences for each of these motifs are provided in the following tables.

Zinc finger linker motif sequences disclosed herein include those of SEQ ID NOs: 1-24:

TGEKP (SEQ ID NO: 1)
TGERP (SEQ ID NO: 2)
TGKKP (SEQ ID NO: 3)
TGKRP (SEQ ID NO: 4)
TGDKP (SEQ ID NO: 5)
TGDRP (SEQ ID NO: 6)
TEEKP (SEQ ID NO: 7)
TEERP (SEQ ID NO: 8)
TEKKP (SEQ ID NO: 9)
TEKRP (SEQ ID NO: 10)
TEDKP (SEQ ID NO: 11)
TEDRP (SEQ ID NO: 12)
SGEKP (SEQ ID NO: 13)
SGERP (SEQ ID NO: 14)
SGKKP (SEQ ID NO: 15)
SGKRP (SEQ ID NO: 16)
SGDKP (SEQ ID NO: 17)
SGDRP (SEQ ID NO: 18)
SEEKP (SEQ ID NO: 19)
SEERP (SEQ ID NO: 20)
SEKKP (SEQ ID NO: 21)
SEKRP (SEQ ID NO: 22)
SEDKP (SEQ ID NO: 23)
SEDRP (SEQ ID NO: 24)

In some embodiments, the present disclosure provides zinc finger proteins comprising one or more linker motifs of SEQ ID NOs: 1-24, or one or more linker motifs comprising an amino acid sequence at least 80%, at least 85%, at least 90%, at least 95%, at least 96%, at least 97%, at least 98%, or at least 99% identical to the amino acid sequence of any one of SEQ ID NOs: 1-24. In some embodiments, a zinc finger domain-containing protein comprises one or more linker motifs comprising the amino acid sequence of any one of TGEKP (SEQ ID NO: 1), SGEKP (SEQ ID NO: 13), SGERP (SEQ ID NO: 14), and SGDKP (SEQ ID NO: 17), or one or more linker motifs comprising an amino acid sequence at least 80%, at least 85%, at least 90%, at least 95%, at least 96%, at least 97%, at least 98%, or at least 99% identical to the amino acid sequence of any one of TGEKP (SEQ ID NO: 1), SGEKP (SEQ ID NO: 13), SGERP (SEQ ID NO: 14), and SGDKP (SEQ ID NO: 17). In certain embodiments, all of the linker motifs present in a zinc finger domain-containing protein each comprise the same amino acid sequence selected from the group consisting of TGEKP (SEQ ID NO: 1), SGEKP (SEQ ID NO: 13), SGERP (SEQ ID NO: 14), and SGDKP (SEQ ID NO: 17), or the same amino acid sequence that is at least 80%, at least 85%, at least 90%, at least 95%, at least 96%, at least 97%, at least 98%, or at least 99% identical to the amino acid sequence of any one of TGEKP (SEQ ID NO: 1), SGEKP (SEQ ID NO: 13), SGERP (SEQ ID NO: 14), and SGDKP (SEQ ID NO: 17).

Zinc Finger α-motif sequences disclosed herein include those of SEQ ID NOs: 25-42 and 346:

HQRIH (SEQ ID NO: 25)
HQRVH (SEQ ID NO: 26)
HQRTH (SEQ ID NO: 27)
HQKIH (SEQ ID NO: 28)
HQKVH (SEQ ID NO: 29)
HQKTH (SEQ ID NO: 30)
HMRIH (SEQ ID NO: 31)
HMRVH (SEQ ID NO: 32)
HMRTH (SEQ ID NO: 33)
HMKIH (SEQ ID NO: 34)
HMKVH (SEQ ID NO: 35)
HMKTH (SEQ ID NO: 36)
HKRIH (SEQ ID NO: 37)
HKRVH (SEQ ID NO: 38)
HKRTH (SEQ ID NO: 39)
HKKIH (SEQ ID NO: 40)
HKKVH (SEQ ID NO: 41)
HKKTH (SEQ ID NO: 42)
HIRTH (SEQ ID NO: 346)

In some embodiments, the present disclosure provides zinc finger proteins comprising one or more alpha motifs of SEQ ID NOs: 25-42 and 346, or one or more alpha motifs comprising an amino acid sequence at least 80%, at least 85%, at least 90%, at least 95%, at least 96%, at least 97%, at least 98%, or at least 99% identical to the amino acid sequence of any one of SEQ ID NOs: 25-42 and 346. In some embodiments, a zinc finger domain-containing protein comprises one or more α-motifs comprising the amino acid sequence of any one of HMRTH (SEQ ID NO: 33), HMKIH (SEQ ID NO: 34), HMKVH (SEQ ID NO: 35), HMKTH (SEQ ID NO: 36), and HIRTH (SEQ ID NO: 346), or one or more alpha motifs comprising an amino acid sequence at least 80%, at least 85%, at least 90%, at least 95%, at least 96%, at least 97%, at least 98%, or at least 99% identical to the amino acid sequence of any one of HMRTH (SEQ ID NO: 33), HMKIH (SEQ ID NO: 34), HMKVH (SEQ ID NO: 35), HMKTH (SEQ ID NO: 36), and HIRTH (SEQ ID NO: 346). In certain embodiments, all of the α-motifs present in a zinc finger domain-containing protein each comprise the same amino acid sequence selected from the group consisting of HMRTH (SEQ ID NO: 33), HMKIH (SEQ ID NO: 34), HMKVH (SEQ ID NO: 35), HMKTH (SEQ ID NO: 36), and HIRTH (SEQ ID NO: 346), or the same amino acid sequence that is at least 80%, at least 85%, at least 90%, at least 95%, at least 96%, at least 97%, at least 98%, or at least 99% identical to the amino acid sequence of any one of HMRTH (SEQ ID NO: 33), HMKJH (SEQ ID NO: 34), HMKVH (SEQ ID NO: 35), HMKTH (SEQ ID NO: 36), and HIRTH (SEQ ID NO: 346).

Zinc Finger β-motif sequences disclosed herein include those of SEQ ID NOs: 43-138 and 336-345:

YKCKECGKAFS (SEQ ID NO: 43)
YKCKECGKAFR (SEQ ID NO: 44)
YKCKECGKAFN (SEQ ID NO: 45)
YKCKECGKSFS (SEQ ID NO: 46)
YKCKECGKSFR (SEQ ID NO: 47)
YKCKECGKSEN (SEQ ID NO: 48)
YKCNECGKAFS (SEQ ID NO: 49)
YKCNECGKAFR (SEQ ID NO: 50)
YKCNECGKAFN (SEQ ID NO: 51)
YKCNECGKSFS (SEQ ID NO: 52)
YKCNECGKSFR (SEQ ID NO: 53)
YKCNECGKSEN (SEQ ID NO: 54)
YKCSECGKAFS (SEQ ID NO: 55)
YKCSECGKAFR (SEQ ID NO: 56)
YKCSECGKAFN (SEQ ID NO: 57)
YKCSECGKSFS (SEQ ID NO: 58)
YKCSECGKSFR (SEQ ID NO: 59)
YKCSECGKSEN (SEQ ID NO: 60)
YKCEECGKAFS (SEQ ID NO: 61)
YKCEECGKAFR (SEQ ID NO: 62)
YKCEECGKAFN (SEQ ID NO: 63)
YKCEECGKSFS (SEQ ID NO: 64)
YKCEECGKSFR (SEQ ID NO: 65)
YKCEECGKSEN (SEQ ID NO: 66)
YECKECGKAFS (SEQ ID NO: 67)
YECKECGKAFR (SEQ ID NO: 68)
YECKECGKAFN (SEQ ID NO: 69)
YECKECGKSFS (SEQ ID NO: 70)
YECKECGKSFR (SEQ ID NO: 71)
YECKECGKSEN (SEQ ID NO: 72)
YECNECGKAFS (SEQ ID NO: 73)
YECNECGKAFR (SEQ ID NO: 74)
YECNECGKAFN (SEQ ID NO: 75)
YECNECGKSFS (SEQ ID NO: 76)
YECNECGKSFR (SEQ ID NO: 77)
YECNECGKSEN (SEQ ID NO: 78)
YECSECGKAFS (SEQ ID NO: 79)
YECSECGKAFR (SEQ ID NO: 80)
YECSECGKAFN (SEQ ID NO: 81)
YECSECGKSFS (SEQ ID NO: 82)
YECSECGKSFR (SEQ ID NO: 83)
YECSECGKSFN (SEQ ID NO: 84)
YECEECGKAFS (SEQ ID NO: 85)
YECEECGKAFR (SEQ ID NO: 86)
YECEECGKAFN (SEQ ID NO: 87)
YECEECGKSFS (SEQ ID NO: 88)
YECEECGKSFR (SEQ ID NO: 89)
YECEECGKSEN (SEQ ID NO: 90)
FKCKECGKAFS (SEQ ID NO: 91)
FKCKECGKAFR (SEQ ID NO: 92)
FKCKECGKAFN (SEQ ID NO: 93)
FKCKECGKSFS (SEQ ID NO: 94)
FKCKECGKSFR (SEQ ID NO: 95)
FKCKECGKSFN (SEQ ID NO: 96)
FKCNECGKAFS (SEQ ID NO: 97)
FKCNECGKAFR (SEQ ID NO: 98)
FKCNECGKAFN (SEQ ID NO: 99)
FKCNECGKSFS (SEQ ID NO: 100)
FKCNECGKSFR (SEQ ID NO: 101)
FKCNECGKSEN (SEQ ID NO: 102)
FKCSECGKAFS (SEQ ID NO: 103)
FKCSECGKAFR (SEQ ID NO: 104)
FKCSECGKAFN (SEQ ID NO: 105)
FKCSECGKSFS (SEQ ID NO: 106)
FKCSECGKSFR (SEQ ID NO: 107)
FKCSECGKSFN (SEQ ID NO: 108)
FKCEECGKAFS (SEQ ID NO: 109)
FKCEECGKAFR (SEQ ID NO: 110)
FKCEECGKAFN (SEQ ID NO: 111)
FKCEECGKSFS (SEQ ID NO: 112)
FKCEECGKSFR (SEQ ID NO: 113)
FKCEECGKSEN (SEQ ID NO: 114)
FECKECGKAFS (SEQ ID NO: 115)
FECKECGKAFR (SEQ ID NO: 116)
FECKECGKAFN (SEQ ID NO: 117)
FECKECGKSFS (SEQ ID NO: 118)
FECKECGKSFR (SEQ ID NO: 119)
FECKECGKSEN (SEQ ID NO: 120)
FECNECGKAFS (SEQ ID NO: 121)
FECNECGKAFR (SEQ ID NO: 122)
FECNECGKAFN (SEQ ID NO: 123)
FECNECGKSFS (SEQ ID NO: 124)
FECNECGKSFR (SEQ ID NO: 125)
FECNECGKSEN (SEQ ID NO: 126)
FECSECGKAFS (SEQ ID NO: 127)
FECSECGKAFR (SEQ ID NO: 128)
FECSECGKAFN (SEQ ID NO: 129)
FECSECGKSFS (SEQ ID NO: 130)
FECSECGKSFR (SEQ ID NO: 131)
FECSECGKSEN (SEQ ID NO: 132)
FECEECGKAFS (SEQ ID NO: 133)
FECEECGKAFR (SEQ ID NO: 134)
FECEECGKAFN (SEQ ID NO: 135)
FECEECGKSFS (SEQ ID NO: 136)
FECEECGKSFR (SEQ ID NO: 137)
FECEECGKSEN (SEQ ID NO: 138)
YKCPECGKSFS (SEQ ID NO: 336)
YACPECGKSFS (SEQ ID NO: 337)
YACPECGRSFS (SEQ ID NO: 338)
YACPECDRSES (SEQ ID NO: 339)
YACPECDRSFS (SEQ ID NO: 340)
YACPECDRRES (SEQ ID NO: 341)
YACPVESCDRRFS (SEQ ID NO: 342)
YACPVESCDRSFS (SEQ ID NO: 343)
YACPVESCGKSFS (SEQ ID NO: 344)
FACDICGRKFA (SEQ ID NO: 345)

In some embodiments, the present disclosure provides zinc finger proteins comprising one or more beta motifs of SEQ ID NOs: 43-138 and 336-345, or one or more beta motifs comprising an amino acid sequence at least 80%, at least 85%, at least 90%, at least 95%, at least 96%, at least 97%, at least 98%, or at least 99% identical to the amino acid sequence of any one of SEQ ID NOs: 43-138 and 336-345. In some embodiments, a zinc finger domain-containing protein comprises one or more β-motifs comprising the amino acid sequence of any one of YKCNECGKAFN (SEQ ID NO: 51), YKCNECGKSFN (SEQ ID NO: 54), YKCSECGKAFN (SEQ ID NO: 57), YKCEECGKAFN (SEQ ID NO: 63), FKCNECGKAFN (SEQ ID NO: 99), FKCNECGKSFN (SEQ ID NO: 102), FKCSECGKAFN (SEQ ID NO: 105), FKCEECGKAFS (SEQ ID NO: 109), FKCEECGKAFN (SEQ ID NO: 111), FKCEECGKSFN (SEQ ID NO: 114), YACPECGKSFS (SEQ ID NO: 337), and FACDICGRKFA (SEQ ID NO: 345), or an amino acid sequence at least 80%, at least 85%, at least 90%, at least 95%, at least 96%, at least 97%, at least 98%, or at least 99% identical to any one of YKCNECGKAFN (SEQ ID NO: 51), YKCNECGKSFN (SEQ ID NO: 54), YKCSECGKAFN (SEQ ID NO: 57), YKCEECGKAFN (SEQ ID NO: 63), FKCNECGKAFN (SEQ ID NO: 99), FKCNECGKSFN (SEQ ID NO: 102), FKCSECGKAFN (SEQ ID NO: 105), FKCEECGKAFS (SEQ ID NO: 109), FKCEECGKAFN (SEQ ID NO: 111), FKCEECGKSFN (SEQ ID NO: 114), YACPECGKSFS (SEQ ID NO: 337), and FACDICGRKFA (SEQ ID NO: 345). In certain embodiments, all of the β-motifs present in a zinc finger domain-containing protein each comprise the same amino acid sequence selected from the group consisting of YKCNECGKAFN (SEQ ID NO: 51), YKCNECGKSFN (SEQ ID NO: 54), YKCSECGKAFN (SEQ ID NO: 57), YKCEECGKAFN (SEQ ID NO: 63), FKCNECGKAFN (SEQ ID NO: 99), FKCNECGKSFN (SEQ ID NO: 102), FKCSECGKAFN (SEQ ID NO: 105), FKCEECGKAFS (SEQ ID NO: 109), FKCEECGKAFN (SEQ ID NO: 111), FKCEECGKSFN (SEQ ID NO: 114), YACPECGKSFS (SEQ ID NO: 337), and FACDICGRKFA (SEQ ID NO: 345), or the same amino acid sequence that is at least 80%, at least 85%, at least 90%, at least 95%, at least 96%, at least 97%, at least 98%, or at least 99% identical to any one of YKCNECGKAFN (SEQ ID NO: 51), YKCNECGKSFN (SEQ ID NO: 54), YKCSECGKAFN (SEQ ID NO: 57), YKCEECGKAFN (SEQ ID NO: 63), FKCNECGKAFN (SEQ ID NO: 99), FKCNECGKSFN (SEQ ID NO: 102), FKCSECGKAFN (SEQ ID NO: 105), FKCEECGKAFS (SEQ ID NO: 109), FKCEECGKAFN (SEQ ID NO: 111), FKCEECGKSFN (SEQ ID NO: 114), YACPECGKSFS (SEQ ID NO: 337), and FACDICGRKFA (SEQ ID NO: 345).

Thus, in one aspect, the present disclosure provides zinc finger domain-containing proteins comprising (i) one or more linker motifs, wherein each linker motif independently comprises the amino acid sequence of any one of SEQ ID NOs: 1-24; (ii) one or more α-motifs, wherein each α-motif independently comprises the amino acid sequence of any one of SEQ ID NOs: 25-42 and 346; and (iii) one or more β-motifs, wherein each β-motif independently comprises the amino acid sequence of any one of SEQ ID NOs: 43-138 and 336-345, or an amino acid sequence that is at least 90%, at least 95%, or at least 99% identical to the amino acid sequence of any one of SEQ ID NOs: 43-138 and 336-345.

Zinc finger proteins consist of repeating subunits of the general structure [β-motif]-[DNA recognition motif]-[α-motif]joined together by a linker motif. Zinc finger proteins generally comprise at least three repeats of this general structure. In some embodiments, a zinc finger protein comprises three repeats of this general structure. In some embodiments, a zinc finger protein comprises four repeats of this general structure. In some embodiments, a zinc finger protein comprises five repeats of this general structure. In some embodiments, a zinc finger protein comprises six repeats of this general structure. In certain embodiments, a zinc finger domain-containing protein comprises any of the following structures:
[first β-motif]-[first DNA recognition motif]-[first α-motif]-[first linker motif]-[second β-motif]-[second DNA recognition motif]-[second α-motif]-[second linker motif]-[third β-motif]-[third DNA recognition motif]-[third α-motif];
[first β-motif]-[first DNA recognition motif]-[first α-motif]-[first linker motif]-[second β-motif]-[second DNA recognition motif]-[second α-motif]-[second linker motif]-[third β-motif]-[third DNA recognition motif]-[third α-motif]-[third linker motif]-[fourth β-motif]-[fourth DNA recognition motif]-[fourth α-motif];
[first β-motif]-[first DNA recognition motif]-[first α-motif]-[first linker motif]-[second β-motif]-[second DNA recognition motif]-[second α-motif]-[second linker motif]-[third β-motif]-[third DNA recognition motif]-[third α-motif]-[third linker motif]-[fourth β-motif]-[fourth DNA recognition motif]-[fourth α-motif]-[fourth linker motif]-[fifth β-motif]-[fifth DNA recognition motif]-[fifth α-motif]; or
[first β-motif]-[first DNA recognition motif]-[first α-motif]-[first linker motif]-[second β-motif]-[second DNA recognition motif]-[second α-motif]-[second linker motif]-[third β-motif]-[third DNA recognition motif]-[third α-motif]-[third linker motif]-[fourth β-motif]-[fourth DNA recognition motif]-[fourth α-motif]-[fourth linker motif]-[fifth β-motif]-[fifth DNA recognition motif]-[fifth α-motif]-[fifth linker motif]-[sixth β-motif]-[sixth DNA recognition motif]-[sixth α-motif].

Any of the zinc finger domain-containing proteins provided herein may further comprise an N-terminal cap. In some embodiments, an N-terminal cap comprises the amino acid sequence MAERP. Thus, in certain embodiments, a zinc finger domain-containing protein may comprise any of the following structures:
[N-terminal cap]-[first β-motif]-[first DNA recognition motif]-[first α-motif]-[first linker motif]-[second β-motif]-[second DNA recognition motif]-[second α-motif]-[second linker motif]-[third β-motif]-[third DNA recognition motif]-[third α-motif];
[N-terminal cap]-[first β-motif]-[first DNA recognition motif]-[first α-motif]-[first linker motif]-[second β-motif]-[second DNA recognition motif]-[second α-motif]-[second linker motif]-[third β-motif]-[third DNA recognition motif]-[third α-motif]-[third linker motif]-[fourth β-motif]-[fourth DNA recognition motif]-[fourth α-motif];
[N-terminal cap]-[first β-motif]-[first DNA recognition motif]-[first α-motif]-[first linker motif]-[second β-motif]-[second DNA recognition motif]-[second α-motif]-[second linker motif]-[third β-motif]-[third DNA recognition motif]-[third α-motif]-[third linker motif]-[fourth β-motif]-[fourth DNA recognition motif]-[fourth α-motif]-[fourth linker motif]-[fifth β-motif]-[fifth DNA recognition motif]-[fifth α-motif]; or
[N-terminal cap]-[first β-motif]-[first DNA recognition motif]-[first α-motif]-[first linker motif]-[second β-motif]-[second DNA recognition motif]-[second α-motif]-[second linker motif]-[third β-motif]-[third DNA recognition motif]-[third α-motif]-[third linker motif]-[fourth β-motif]-[fourth DNA recognition motif]-[fourth α-motif]-[fourth linker motif]-[fifth β-motif]-[fifth DNA recognition motif]-[fifth α-motif]-[fifth linker motif]-[sixth β-motif]-[sixth DNA recognition motif]-[sixth α-motif].

Any of the zinc finger domain-containing proteins provided herein may also further comprise a C-terminal cap. In some embodiments a C-terminal cap comprises the amino acid sequence HTKIHLR. Thus, in certain embodiments, a zinc finger domain-containing protein may comprise any of the following structures:
[first β-motif]-[first DNA recognition motif]-[first α-motif]-[first linker motif]-[second β-motif]-[second DNA recognition motif]-[second α-motif]-[second linker motif]-[third β-motif]-[third DNA recognition motif]-[third α-motif]-[C-terminal cap];
[first β-motif]-[first DNA recognition motif]-[first α-motif]-[first linker motif]-[second β-motif]-[second DNA recognition motif]-[second α-motif]-[second linker motif]-[third β-motif]-[third DNA recognition motif]-[third α-motif]-[third linker motif]-[fourth β-motif]-[fourth DNA recognition motif]-[fourth α-motif]-[C-terminal cap];
[first β-motif]-[first DNA recognition motif]-[first α-motif]-[first linker motif]-[second β-motif]-[second DNA recognition motif]-[second α-motif]-[second linker motif]-[third β-motif]-[third DNA recognition motif]-[third α-motif]-[third linker motif]-[fourth β-motif]-[fourth DNA recognition motif]-[fourth α-motif]-[fourth linker motif]-[fifth β-motif]-[fifth DNA recognition motif]-[fifth α-motif]-[C-terminal cap]; or
[first β-motif]-[first DNA recognition motif]-[first α-motif]-[first linker motif]-[second β-motif]-[second DNA recognition motif]-[second α-motif]-[second linker motif]-[third β-motif]-[third DNA recognition motif]-[third α-motif]-[third linker motif]-[fourth β-motif]-[fourth DNA recognition motif]-[fourth α-motif]-[fourth linker motif]-[fifth β-motif]-[fifth DNA recognition motif]-[fifth α-motif]-[fifth linker motif]-[sixth β-motif]-[sixth DNA recognition motif]-[sixth α-motif]-[C-terminal cap].

In certain embodiments, any of the zinc finger domain-containing proteins provided herein may comprise both an N-terminal cap (e.g., MAERP) and a C-terminal cap (e.g., HTKIHLR). Thus, in certain embodiments, a zinc finger domain-containing protein may comprise any of the following structures:
[N-terminal cap]-[first β-motif]-[first DNA recognition motif]-[first α-motif]-[first linker motif]-[second β-motif]-[second DNA recognition motif]-[second α-motif]-[second linker motif]-[third β-motif]-[third DNA recognition motif]-[third α-motif]-[C-terminal cap];
[N-terminal cap]-[first β-motif]-[first DNA recognition motif]-[first α-motif]-[first linker motif]-[second β-motif]-[second DNA recognition motif]-[second α-motif]-[second linker motif]-[third β-motif]-[third DNA recognition motif]-[third α-motif]-[third linker motif]-[fourth β-motif]-[fourth DNA recognition motif]-[fourth α-motif]-[C-terminal cap];
[N-terminal cap]-[first β-motif]-[first DNA recognition motif]-[first α-motif]-[first linker motif]-[second β-motif]-[second DNA recognition motif]-[second α-motif]-[second linker motif]-[third β-motif]-[third DNA recognition motif]-[third α-motif]-[third linker motif]-[fourth β-motif]-[fourth DNA recognition motif]-[fourth α-motif]-[fourth linker motif]-[fifth β-motif]-[fifth DNA recognition motif]-[fifth α-motif]-[C-terminal cap]; or
[N-terminal cap]-[first β-motif]-[first DNA recognition motif]-[first α-motif]-[first linker motif]-[second β-motif]-[second DNA recognition motif]-[second α-motif]-[second linker motif]-[third β-motif]-[third DNA recognition motif]-[third α-motif]-[third linker motif]-[fourth β-motif]-[fourth DNA recognition motif]-[fourth α-motif]-[fourth linker motif]-[fifth β-motif]-[fifth DNA recognition motif]-[fifth α-motif]-[fifth linker motif]-[sixth β-motif]-[sixth DNA recognition motif]-[sixth α-motif]-[C-terminal cap].

Each of the linker, alpha, and beta motifs may comprise or consist of any of the various amino acid sequences provided herein, in any combination with one another. In certain embodiments, the present disclosure provides zinc finger proteins wherein each of the linker motifs present in the protein comprises the same amino acid sequence, each of the alpha-motifs present in the protein comprises the same amino acid sequence, and each of the beta-motifs present in the protein comprises the same amino acid sequence. For example, in some embodiments, the present disclosure provides zinc finger proteins comprising three repeating zinc finger motifs wherein each of the first, second, and third β-motifs comprise the same amino acid sequence, each of the first, second, and third α-motifs comprise the same amino acid sequence, and/or each of the first and second linker motifs comprise the same amino acid sequence. In some embodiments, the present disclosure provides zinc finger proteins comprising four repeating zinc finger motifs wherein each of the first, second, third, and fourth β-motifs comprise the same amino acid sequence, each of the first, second, third, and fourth α-motifs comprise the same amino acid sequence, and/or each of the first, second, and third linker motifs comprise the same amino acid sequence. In some embodiments, the present disclosure provides zinc finger proteins comprising five repeating zinc finger motifs wherein each of the first, second, third, fourth, and fifth β-motifs comprise the same amino acid sequence, each of the first, second, third, fourth, and fifth α-motifs comprise the same amino acid sequence, and/or each of the first, second, third, and fourth linker motifs comprise the same amino acid sequence. In some embodiments, the present disclosure provides zinc finger proteins comprising six repeating zinc finger motifs wherein each of the first, second, third, fourth, fifth, and sixth β-motifs comprise the same amino acid sequence, each of the first, second, third, fourth, fifth, and sixth α-motifs comprise the same amino acid sequence, and each of the first, second, third, fourth, and fifth linker motifs comprise the same amino acid sequence.

In certain embodiments, the present disclosure provides zinc finger domain-containing proteins in which every β-motif comprises the amino acid sequence FACDICGRKFA (SEQ ID NO: 345), every α-motif comprises the amino acid sequence HIRTH (SEQ ID NO: 346), and every linker motif comprises the amino acid sequence TGEKP (SEQ ID NO: 1). In certain embodiments, every β-motif comprises the amino acid sequence YACPECGKSFS (SEQ ID NO: 337), every α-motif comprises the amino acid sequence HIRTH (SEQ ID NO: 346), and every linker motif comprises the amino acid sequence TGEKP (SEQ ID NO: 1). In certain embodiments, every β-motif comprises the amino acid sequence FKCEECGKAFN (SEQ ID NO: 111), every α-motif comprises the amino acid sequence HIRTH (SEQ ID NO: 346), and every linker motif comprises the amino acid sequence TGEKP (SEQ ID NO: 1). In certain embodiments, every β-motif comprises the amino acid sequence YKCEECGKAFN (SEQ ID NO: 63), every α-motif comprises the amino acid sequence HIRTH (SEQ ID NO: 346), and every linker motif comprises the amino acid sequence TGEKP (SEQ ID NO: 1).

The DNA-binding domains of individual zinc finger proteins typically contain between three and six individual zinc finger motifs (each containing a β-motif, a DNA recognition motif, and an α-motif, as described above) each connected to one another by a linker motif. Each zinc finger protein can typically recognize between 9 and 18 base pairs. For example, a zinc finger protein comprising an array of three zinc finger motifs will typically recognize a nine-nucleotide sequence. A zinc finger protein comprising an array of four zinc finger motifs will typically recognize a twelve-nucleotide sequence. A zinc finger protein comprising an array of five zinc finger motifs will typically recognize a fifteen-nucleotide sequence. And a zinc finger protein comprising an array of six zinc finger motifs will typically recognize an eighteen-nucleotide sequence.

Amino acid sequences of various zinc finger DNA-binding domains that recognize particular three-nucleotide DNA sequences have been characterized and are well known in the art. These variable amino acid sequences generally contain seven amino acid residues that can recognize and interact with (e.g., bind to) specific nucleotide sequences (generally of three nucleotides in length). The seven variable DNA-binding residues (typically numbered from −1 to 6) are inserted in between the beta-motif and the alpha-motif within each individual ZF repeat and vary between each individual ZF repeat depending on the target DNA sequence. The variable DNA-binding residues are therefore distinct from, and do not overlap with, the beta-motif and the alpha-motif sequences. For example, the following seven-amino acid DNA recognition sequences that recognize particular three-nucleotide DNA sequences may be used in the ZF domain-containing proteins described herein:

| Target DNA | | | ZF nt |
|---|---|---|---|
| sequence | ZF amino acid | ZF nucleotide | sequence |
| (5′ to 3′) | sequence | sequence | SEQ ID NO: |
| AAA | QRANLRA (SEQ ID NO: | cagagagctaatctcagggcc | 816 |
| | 753) | | |
| AAC | DSGNLRV (SEQ ID NO: | gattcagggaatctccgggtt | 817 |
| | 754) | | |
| AAG | RKDNLKN (SEQ ID NO: | cgaaaagataatctgaagaat | 818 |
| | 755) | | |
| AAT | TTGNLTV (SEQ ID NO: | accactggaaacctcacggtg | 819 |
| | 756) | | |
| ACA | SPADLTR (SEQ ID NO: | agtcctgcagatcttacccga | 820 |
| | 757) | | |
| ACC | DKKDLTR (SEQ ID NO: | gacaagaaggatctgacacga | 821 |
| | 758) | | |
| ACG | RTDTLRD (SEQ ID NO: | aggactgatacgctgcgcgat | 822 |
| | 759) | | |
| ACT | THLDLIR (SEQ ID NO: | acccacctggacctcatcaga | 823 |
| | 760) | | |
| AGA | QLAHLRA (SEQ ID NO: | caactcgctcatctgcgagca | 824 |
| | 761) | | |
| AGC | ERSHLRE (SEQ ID NO: | gaacgaagccacctgcgcgaa | 825 |
| | 762) | | |
| AGG | RSDHLTN (SEQ ID NO: | cgcagcgaccatttgactaac | 826 |
| | 763) | | |
| AGT | HRTTLTN (SEQ ID NO: | caccgaacgaccttgactaac | 827 |
| | 764) | | |
| ATA | QKSSLIA (SEQ ID NO: | cagaaatcttctttgatagct | 828 |
| | 765) | | |
| ATC | RRSACRR (SEQ ID NO: | cggagatcagcctgtcgacgc | 829 |
| | 766) | | |
| ATG | RRDELNV (SEQ ID NO: | aggcgggacgaactgaacgtg | 830 |
| | 767) | | |
| ATT | HKNALQN (SEQ ID NO: | cacaaaaatgccttgcaaaac | 831 |
| | 768) | | |
| CAA | QSGNLTE (SEQ ID NO: | caatctggcaatcttacagag | 832 |
| | 769) | | |
| CAC | SKKALTE (SEQ ID NO: | tctaaaaaggcgctgacggag | 833 |
| | 770) | | |
| CAG | RADNLTE (SEQ ID NO: | cgggcggataatctcactgag | 834 |
| | 771) | | |
| CAT | TSGNLTE (SEQ ID NO: | acgagtggaaatcttacggaa | 835 |
| | 772) | | |
| CCA | TSHSLTE (SEQ ID NO: | acgtcccacagtttgaccgaa | 836 |
| | 773) | | |
| CCC | SKKHLAE (SEQ ID NO: | agcaagaaacaccttgcagaa | 837 |
| | 774) | | |
| CCG | RNDTLTE (SEQ ID NO: | aggaatgatactcttaccgag | 838 |
| | 775) | | |
| CCT | TKNSLTE (SEQ ID NO: | acaaagaacagcctcaccgag | 839 |
| | 776) | | |
| CGA | QSGHLTE (SEQ ID NO: | cagtcagggcatctcacggag | 840 |
| | 777) | | |
| CGC | HTGHLLE (SEQ ID NO: | cacacaggccatttgttggag | 841 |
| | 778) | | |
| CGG | RSDKLTE (SEQ ID NO: | cggagtgataaactcaccgaa | 842 |
| | 779) | | |
| CGT | SRRTCRA (SEQ ID NO: | tcacgacgcacctgtagagcg | 843 |
| | 780) | | |
| CTA | QNSTLTE (SEQ ID NO: | cagaattcaactctcaccgaa | 844 |
| | 781) | | |
| CTC | QRHHLVE (SEQ ID NO: | cagcgacaccatttggtcgag | 845 |
| | 782) | | |
| CTG | RNDALTE (SEQ ID NO: | cggaacgatgcacttaccgag | 846 |
| | 783) | | |
| CTT | TTGALTE (SEQ ID NO: | actacaggggctctcactgaa | 847 |
| | 784) | | |
| GAA | QSSNLVR (SEQ ID NO: | cagagtagtaacctggtgagg | 848 |
| | 785) | | |
| GAC | DPGNLVR (SEQ ID NO: | gatcccgggaacctcgttaga | 849 |
| | 786) | | |
| GAG | RSDNLVR (SEQ ID NO: | cgctctgataacctggtcaga | 850 |
| | 787) | | |
| GAT | TSGNLVR (SEQ ID NO: | actagcgggaacctcgtccgg | 851 |
| | 788) | | |
| GCA | QSGDLRR (SEQ ID NO: | caaagcggggacttgagaagg | 852 |
| | 789) | | |
| GCC | DCRDLAR (SEQ ID NO: | gattgccgagatcttgctcgg | 853 |
| | 790) | | |
| GCG | RSDDLVR (SEQ ID NO: | cgctcagatgatctggttcgc | 854 |
| | 791) | | |
| GCT | TSGELVR (SEQ ID NO: | acgtctggggagttggttagg | 855 |
| | 792) | | |
| GGA | QRAHLER (SEQ ID NO: | caaagagcccatctggaaagg | 856 |
| | 793) | | |
| GGC | DPGHLVR (SEQ ID NO: | gatcccggacacttggttcga | 857 |
| | 794) | | |
| GGG | RSDKLVR (SEQ ID NO: | cgcagcgacaaactcgttaga | 858 |
| | 795) | | |
| GGT | TSGHLVR (SEQ ID NO: | acttcaggccatcttgtaaga | 859 |
| | 796) | | |
| GTA | QSSSLVR (SEQ ID NO: | caatcttcctcacttgtgagg | 860 |
| | 797) | | |
| GTC | DPGALVR (SEQ ID NO: | gacccaggggctttggttcgg | 861 |
| | 798) | | |
| GTG | RSDELVR (SEQ ID NO: | cggtcagatgagctggtacgc | 862 |
| | 799) | | |
| GTT | TSGSLVR (SEQ ID NO: | acaagcggctctctcgttaga | 863 |
| | 800) | | |
| TAA | QASNLIS (SEQ ID NO: | caagcctctaacttgattagc | 864 |
| | 801) | | |
| TAC | SRGNLKS (SEQ ID NO: | agcaggggtaacttgaaatcc | 865 |
| | 802) | | |
| TAG | REDNLHT (SEQ ID NO: | cgggaagacaaccttcatacg | 866 |
| | 803) | | |
| TAT | ARGNLRT (SEQ ID NO: | gcacgcgggaacttgcggact | 867 |
| | 804) | | |
| TCA | RSDHLTT (SEQ ID NO: | cgaagtgatcacttgacaacc | 868 |
| | 811) | | |
| TCC | RSDERKR (SEQ ID NO: | cggtcagacgagagaaagcga | 869 |
| | 806) | | |
| TCG | RLRALDR (SEQ ID NO: | cgcttgcgggcgctcgaccga | 870 |
| | 807) | | |
| TCT | RLRDIQF (SEQ ID NO: | agactcagggatatacaattt | 871 |
| | 808) | | |
| TGA | QAGHLAS (SEQ ID NO: | caaggggccacctcgccagc | 872 |
| | 809) | | |
| TGC | APKALGW (SEQ ID NO: | gccccaaaagcactgggctgg | 873 |
| | 810) | | |
| TGG | RSDHLTT (SEQ ID NO: | cggagcgaccatctcactact | 874 |
| | 811) | | |
| TGT | WRDSLLA (SEQ ID NO: | tggcgcgactcccttctcgcg | 875 |
| | 812) | | |
| TTA | QKWPRDS (SEQ ID NO: | cagaagtggcccagggattca | 876 |
| | 813) | | |
| TTC | DNSYLPR (SEQ ID NO: | gacaattcttacttgcccagg | 877 |
| | 814) | | |
| TTG | RKDALRG (SEQ ID NO: | aggaaagatgcgcttagaggg | 878 |
| | 815) | | |

Several methods to generate a zinc finger array of repeating zinc finger units that each recognize a three-nucleotide sequence have been developed and are known in the art. The most straightforward method to generate new zinc finger arrays is to combine individual zinc finger motifs or shorter zinc finger arrays with known DNA specificity (i.e., “zinc finger modules”) to form longer zinc finger arrays have a particular DNA sequence binding affinity. The concept of obtaining zinc finger DNA binding domains for each of the 64 possible combinations of three-nucleotide sequences and then assembling these domains together to design zinc finger proteins with specificity for any target sequence has been described in the art (see, for example, Pavletich et al. Zinc finger-DNA recognition: crystal structure of a Zif268-DNA complex at 2.1 Å. Science 1991, 252(5007), 809-817, which is incorporated herein by reference). The most common modular assembly process involves combining three separate zinc finger motifs that can each recognize a 3 base pair DNA sequence to generate a zinc finger repeat comprising three zinc finger motifs that can recognize a nine base pair target site. Longer zinc finger arrays that recognize longer target sites can be generated as well, as discussed above. Methods utilizing two zinc finger modules to generate zinc finger arrays comprising up to six individual zinc finger motifs have also been described (see, for example, Shukla et al. Precise genome modification in the crop species Zea mays using zinc finger nucleases. Nature 2009, 459(7245), 437-441, which is incorporated herein by reference). Additionally, variants of the modular assembly approach that take into account the context of neighboring DNA binding domains in the other zinc finger domains within an array have also been described (see, for example, Sander et al. Selection-free zinc finger-nuclease engineering by context-dependent assembly (CoDA). Nature 2011, 8(1), 67-69, which is incorporated herein by reference).

Methods utilizing phage display to select for zinc finger DNA binding domains that recognize a particular DNA sequence have also been developed, as described, e.g., in Segal et al. Toward controlling gene expression at will: selection and design of zinc finger domains recognizing each of the 5′-GNN-3′ DNA target sequences. PNAS 1999, 96(6), 2758-63; Dreier et al. Development of zinc finger domains for recognition of the 5′-CNN-3′ family DNA sequences and their use in the construction of artificial transcription factors. J. Biol. Chem. 2005, 280(42), 35588-35597; and Dreier et al. Development of zinc finger domains for recognition of the 5′-ANN-3′ family of DNA sequences and their use in the construction of artificial transcription factors. J. Biol. Chem. 2001, 276(31), 29466-29478, the contents of each of which are incorporated herein by reference. Methods utilizing yeast one-hybrid systems, bacterial one-hybrid systems, bacterial two-hybrid systems, and mammalian cells have also been developed. For example, a method known as “OPEN” has been developed to select novel three-zinc finger arrays. OPEN utilizes a bacterial two-hybrid system and combines pre-selected pools of individual zinc fingers that have each been selected to recognize and bind to a particular three-nucleotide DNA sequence. A second round of selection is then utilized to obtain three-zinc finger arrays capable of binding a desired nine-nucleotide DNA sequence. The OPEN system is described further in Maeder et al. Rapid “open-source” engineering of customized zinc finger nucleases for highly efficient gene modification. Molecular Cell 2008, 31(2), 294-301, the contents of which are incorporated herein by reference.

Additional references that describe the selection of DNA binding domains to design zinc finger arrays that recognize particular nucleotide sequences (and that describe zinc finger proteins more generally) include, but are not limited to, Hossain et al. Artificial Zinc Finger DNA Binding Domains: Versatile Tools for Genome Engineering and Modulation of Gene Expression. J. Cell Biochem. 2015, 116(11), 2435-2444; Gupta, R. M. and Musunuru, K. Expanding the genetic editing tool kit: ZFNs, TALENs, and CRISPR-Cas9. J. Clin. Invest. 2014, 124(10), 4154-4161; Collin, J. and Lako, M. Concise Review: Putting a Zinc Finger on Stem Cell Biology: Zinc Finger Nuclease-Driven Targeted Genetic Editing in Human Pluripotent Stem Cells. Stem Cells 2011, 29, 1021-1033; Carroll, D. Genome Engineering With Zinc finger Nucleases. Genetics 2011, 188, 773-782; Yang, X. et al. Strategies for mitochondrial gene editing. Comput. Struct. Biotechnol. J. 2021, 19, 3319-3329; Lim et al. Nuclear and mitochondrial DNA editing in human cells with zinc finger deaminases. Nat. Commun. 2022, 13(366); Elrod-Erickson et al. Zif268 protein-DNA complex refined at 1.6 Å: a model system for understanding zinc finger-DNA interactions. Structure 1996, 4(10), 1171-1180; and Jamieson et al. A zinc finger directory for high-affinity DNA recognition. Proc. Natl. Acad. Sci. USA 1996, 93, 12834-12839, each of which is incorporated by reference herein.

### DddA Variants

In some aspects, the present disclosure provides double-stranded DNA deaminase A (DddA) variants. For example, the present disclosure provides DddA variants that exhibit increased on-target editing efficiency and/or decreased off-target editing. As described further herein, the DddA protein is often split into two halves or portions (e.g., at position 1397 of DddA as described herein). The spontaneous reassembly of the two split DddA halves can lead to off-target deamination independent from the on-target site. This can lead to unwanted mutagenesis and increased off-target editing generally if not controlled.

In some embodiments, the DddA variants provided herein are designed to weaken the affinity of the two split DddA halves for one another. Such weaking of the interaction between the two DddA portions allows for fine-tuning of the deaminase activity to eliminate its off-target activity while still preserving high on-target editing efficiency.

In various embodiments involving obtaining a DddA variant by way of one or more methodologies, such as, but not limited to, mutagenesis (e.g., through alanine scanning, lysine scanning, glutamate scanning, and/or aspartate scanning), protein truncation or elongation, and insertion of charged residues into a linker upstream of DddA (e.g., in the context of a fusion protein, such as the base editors described herein), the process may begin with a “starter” protein, such as canonical DddA or a fragment of DddA.

In various embodiments, the starter DddA protein from which variants are derived can be the canonical protein, or a fragment thereof. As reported in Mok et al. 2020, DddA was discovered in Burkholderia cenocepia and reported in the Protein Data Bank as PDB ID: 6U08, which has the following full-length amino acid sequence (1427 amino acids):

>tr|A0A1V6L4E7|A0A1V6L4E7_9BURK YD repeat (Two copies)
OS = Burkholderia cenocepacia OX = 95486 GN = UE95_03830
PE = 1 SV = 1
(SEQ ID NO: 356)
MYEAARVTDPIDHTSALAGFLVGAVLGIALIAAVAFATFTCGFGVALLAGMMAGIGAQ
ALLSIGESIGKMFSSQSGNIITGSPDVYVNSLSAAYATLSGVACSKHNPIPLVAQGSTNIFI
NGRPAARKDDKITCGATIGDGSHDTFFHGGTQTYLPVDDEVPPWLRTATDWAFTLAGL
VGGLGGLLKASGGLSRAVLPCAAKFIGGYVLGEAFGRYVAGPAINKAIGGLFGNPIDVT
TGRKILLAESETDYVIPSPLPVAIKRFYSSGIDYAGTLGRGWVLPWEIRLHARDGRLWYT
DAQGRESGFPMLRAGQAAFSEADQRYLTRTPDGRYILHDLGERYYDFGQYDPESGRIA
WVRRVEDQAGQWYQFERDSRGRVTEILTCGGLRAVLDYETVFGRLGTVTLVHEDERRL
AVTYGYDENGQLASVTDANGAVVRQFAYTNGLMTSHMNALGFTSSYVWSKIEGEPRV
VETHTSEGENWTFEYDVAGRQTRVRHADGRTAHWRFDAQSQIVEYTDLDGAFYRIKY
DAVGMPVMLMLPGDRTVMFEYDDAGRIIAETDPLGRTTRTRYDGNSLRPVEVVGPDGG
AWRVEYDQQGRVVSNQDSLGRENRYEYPKALTALPSAHIDALGGRKTLEWNSLGKLV
GYTDCSGKTTRTSFDAFGRICSRENALGQRITYDVRPTGEPRRVTYPDGSSETFEYDAAG
TLVRYIGLGGRVQELLRNARGQLIEAVDPAGRRVQYRYDVEGRLRELQQDHARYTFTY
SAGGRLLTETRPDGILRRFEYGEAGELLGLDIVGAPDPHATGNRSVRTIRFERDRMGVLK
VQRTPTEVTRYQHDKGDRLVKVERVPTPSGIALGIVPDAVEFEYDKGGRLVAEHGSNGS
VIYTLDELDNVVSLGLPHDQTLQMLRYGSGHVHQIRFGDQVVADFERDDLHREVSRTQ
GRLTQRSGYDPLGRKVWQSAGIDPEMLGRGSGQLWRNYGYDAAGDLIETSDSLRGSTR
FSYDPAGRLISRANPLDRKFEEFAWDAAGNLLDDAQRKSRGYVEGNRLLMWQDLRFE
YDPFGNLATKRRGANQTQRFTYDGQDRLITVHTQDVRGVVETRFAYDPLGRRIAKTDT
AFDLRGMKLRAETKRFVWEGLRLVQEVRETGVSSYVYSPDAPYSPVARADTVMAEAL
AATVIDSAKRAARIFHFHTDPVGAPQEVTDEAGEVAWAGQYAAWGKVEATNRGVTAA
RTDQPLRFAGQYADDSTGLHYNTFRFYDPDVGRFINQDPIGLNGGANVYHYAPNPVGW
VDPWGLAGSYALGPYQISAPQLPAYNGQTVGTFYYVNDAGGLESKVFSSGGPTPYPNY
ANAGHVEGQSALFMRDNGISEGLVFHNNPEGTCGFCVNMTETLLPENAKMTVVPPEGA
IPVKRGATGETKVFTGNSNSPKSPTKGGC.

In various other embodiments, the starter DddA protein can be a split DddA can have the following sequences:
Split DddA (DddA-G1397N) GSYALGPYQISAPQLPAYNGQTVGTFYYVNDAGGLESKVFSSGGPTPYPNYANAGHVE GQSALFMRDNGISEGLVFHNNPEGTCGFCVNMTETLLPENAKMTVVPPEG (SEQ ID NO: 283), and can include fragments or variants thereof, including amino acid sequences having at least 70%, at least 75%, at least 80%, at least 85%, at least 90%, at least 95%, at least 96%, at least 97%, at least 98%, at least 99%, or 100% sequence identify with DddA of SEQ ID NO: 283.

Split DddA (DddA-G1397C)
(SEQ ID NO: 139)
AIPVKRGATGETKVFTGNSNSPKSPTKGGC.

It has been found that the whole, intact DddA protein is toxic to cells. Thus, in order to utilize DddA in the context of the base editors described herein, DddA may be delivered in an inactive form. One of ordinary skill in the art will appreciate that various methods, techniques, and modifications known in the art can be adapted for reversibly inactivating DddA such that the enzyme may be delivered to a cell in an inactive state, but then become activated inside the cell (or the mitochondria) under one or more conditions, or in the presence of one or more inducing agents, in order to conduct the desired deamination.

In preferred embodiments, DddA (including the DddA variants described herein) may be split into inactive fragments that can be separately delivered to a target deamination site on separate fusion constructs that target each fragment of the DddA to sites positioned on either side of a target edit site.

In some embodiments, the DddA variants provided herein comprise a first portion and a second portion. In some embodiments, the first portion and the second portion together comprise a full length DddA. In some embodiments, the first and second portion comprise less than the full length DddA portion. In some embodiments, the first and second portion independently do not have any, or have minimal, native DddA activity (e.g., deamination activity). In some embodiments, the first and second portion can re-assemble (i.e., dimerize) into a DddA protein with (at least partial) native DddA activity (e.g., deamination activity).

In some embodiments, the first and second portion of the DddA are formed by truncating (i.e., dividing or splitting the DddA protein) at specified amino acid residues (e.g., amino acid residue 1397). In some embodiments, the first portion of a DddA comprises a full-length DddA truncated at its N-terminus. In some embodiments, the second portion of a DddA comprises a full-length DddA truncated at its C-terminus. In some embodiments, additional truncations are performed to either the full-length DddA or to the first or second portions of the DddA. In some embodiments, the first and second portions of a DddA may comprise additional truncations, but the first and second portion can dimerize or re-assemble to restore (at least partially) native DddA activity (e.g., deamination).

In certain embodiments, the DddA can be separated into two fragments by dividing the DddA at a split site. A “split site” refers to a position between two adjacent amino acids (in a wildtype DddA amino acid sequence) that marks a point of division of a DddA. In certain embodiments, the DddA can have a least one split site, such that once divided at that split site, the DddA forms an N-terminal fragment and a C-terminal fragment. The N-terminal and C-terminal fragments can be the same or difference sizes (or lengths), wherein the size and/or polypeptide length depends on the location or position of the split site. As used herein, reference to a “fragment” of DddA (or any other polypeptide) can be referred to equivalently as a “portion.” Thus, a DddA that is divided at a split site can form an N-terminal portion and a C-terminal portion. Preferably, the N-terminal fragment (or portion) and the C-terminal fragment (or portion) of DddA do not have deaminase activity on their own, and preferably the N-terminal and C-terminal fragments do have deaminase activity when associated with one another.

In various embodiments, a DddA may be split into two or more inactive fragments by directly cleaving the DddA at one or more split sites. Direct cleaving can be carried out by a protease (e.g., trypsin) or another enzyme or chemical reagent. In certain embodiments, such chemical cleavage reactions can be designed to be site-selective (e.g., Elashal and Raj, “Site-selective chemical cleavage of peptide bonds,” Chemical Communications, 2016, Vol. 52, pages 6304-6307, the contents of which are incorporated herein by reference). In other embodiments, chemical cleavage reactions can be designed to be non-selective and/or occur in a random fashion.

In other embodiments, the two or more inactive DddA fragments can be engineered as separately expressed polypeptides. For instance, for a DddA having one split site, the N-terminal DddA fragment could be engineered from a first nucleotide sequence that encodes the N-terminal DddA fragment (which extends from the N-terminus of the DddA up to and including the residue on the amino-terminal side of the split site). In such an example, the C-terminal DddA fragment could be engineered from a second nucleotide sequence that encodes the C-terminal DddA fragment (which extends from the carboxy-terminus of the split site up to including the natural C-terminus of the DddA protein). The first and second nucleotide sequences could be on the same or different nucleotide molecules (e.g., the same or different expression vectors).

In various embodiments, the N-terminal portion of the DddA variants provided herein may be referred to as “DddA-N half” and the C-terminal portion of the DddA variants provided herein may be referred to as the “DddA-C half.” Reference to the term “half” does not connote the requirement that the DddA-N and DddA-C portions are identically half of the size and/or sequence length of a complete DddA, or that the split site is required to be at the midpoint of the complete DddA polypeptide. To the contrary, and as noted above, the split site can be between any pair of residues in the DddA polypeptide, thereby giving rise to half portions that are unequal in size and/or sequence length. In certain embodiments, the split site is within a loop region of the DddA.

In one aspect, the present disclosure provides DddA variants comprising a first fragment comprising an amino acid sequence that is at least 80%, at least 85%, at least 90%, at least 95%, or at least 99% identical to the amino acid sequence of SEQ ID NO: 139, and a second fragment comprising an amino acid sequence that is at least 80%, at least 85%, at least 90%, at least 95%, or at least 99% identical to the amino acid sequence of SEQ ID NO: 283, wherein the first fragment comprises one or more amino acid substitutions, truncations, or extensions relative to the amino acid sequence of SEQ ID NO: 139, and/or wherein the second fragment comprises one or more amino acid substitutions, truncations, or extensions relative to the amino acid sequence of SEQ ID NO: 283.

In some embodiments, the DddA variants provided herein comprise point mutations relative to a wild type DddA sequence. As described further herein, it was hypothesized by the inventors that introduction of individual point mutations in the C-terminal DddA fragment (G1397C) would reduce the interaction interface between the two split DddA halves and weaken the spontaneous reassembly of DddA at off-target sites. Thus, alanine scanning (to remove side chain interactions), lysine scanning (to introduce positive charge), and glutamate and aspartate scanning (to introduce negative charge) were performed. In this way, 120 constructs were tested in which each of the 30 residues in the C-terminal DddA fragment (G1397C) was individually mutated to either Ala, Lys, Glu or Asp. In some embodiments, the present disclosure provides DddA point mutants that exhibit lower off-target editing without an observed decrease in on-target editing, or point mutants that exhibit large reductions in off-target editing with only minor decreases in on-target editing. Such exemplary point mutants include DddA variants with amino acid substitutions at positions A5, A6, A7, A9, A14, A25, K12, K14, K18, K25, D3, D4, D5, D9, D14, DA, D19, D20, D25, D27, E5, E13, E16 and E20.

Exemplary DddA point mutants provided by the present disclosure include those comprising the following point mutations in the DddA C-terminal fragment G1397C:

| Mutation: | Sequence: |
|---|---|
| Canonical | AIPVKRGATGETKVFTGNSNSPKSPTKGGC (SEQ ID NO: 139) |
| I2A | AAPVKRGATGETKVFTGNSNSPKSPTKGGC (SEQ ID NO: 140) |
| P3A | AIAVKRGATGETKVFTGNSNSPKSPTKGGC (SEQ ID NO: 141) |
| V4A | AIPAKRGATGETKVFTGNSNSPKSPTKGGC (SEQ ID NO: 142) |
| K5A | AIPVARGATGETKVFTGNSNSPKSPTKGGC (SEQ ID NO: 143) |
| R6A | AIPVKAGATGETKVFTGNSNSPKSPTKGGC (SEQ ID NO: 144) |
| G7A | AIPVKRAATGETKVFTGNSNSPKSPTKGGC (SEQ ID NO: 145) |
| T9A | AIPVKRGAAGETKVFTGNSNSPKSPTKGGC (SEQ ID NO: 146) |
| G10A | AIPVKRGATAETKVFTGNSNSPKSPTKGGC (SEQ ID NO: 147) |
| E11A | AIPVKRGATGATKVFTGNSNSPKSPTKGGC (SEQ ID NO: 148) |
| T12A | AIPVKRGATGEAKVFTGNSNSPKSPTKGGC (SEQ ID NO: 149) |
| K13A | AIPVKRGATGETAVFTGNSNSPKSPTKGGC (SEQ ID NO: 150) |
| V14A | AIPVKRGATGETKAFTGNSNSPKSPTKGGC (SEQ ID NO: 151) |
| F15A | AIPVKRGATGETKVATGNSNSPKSPTKGGC (SEQ ID NO: 152) |
| T16A | AIPVKRGATGETKVFAGNSNSPKSPTKGGC (SEQ ID NO: 153) |
| G17A | AIPVKRGATGETKVFTANSNSPKSPTKGGC (SEQ ID NO: 154) |
| N18A | AIPVKRGATGETKVFTGASNSPKSPTKGGC (SEQ ID NO: 155) |
| S19A | AIPVKRGATGETKVFTGNANSPKSPTKGGC (SEQ ID NO: 156) |
| N20A | AIPVKRGATGETKVFTGNSASPKSPTKGGC (SEQ ID NO: 157) |
| S21A | AIPVKRGATGETKVFTGNSNAPKSPTKGGC (SEQ ID NO: 158) |
| P22A | AIPVKRGATGETKVFTGNSNSAKSPTKGGC (SEQ ID NO: 159) |
| K23A | AIPVKRGATGETKVFTGNSNSPASPTKGGC (SEQ ID NO: 160) |
| S24A | AIPVKRGATGETKVFTGNSNSPKAPTKGGC (SEQ ID NO: 161) |
| P25A | AIPVKRGATGETKVFTGNSNSPKSATKGGC (SEQ ID NO: 162) |
| T26A | AIPVKRGATGETKVFTGNSNSPKSPAKGGC (SEQ ID NO: 163) |
| K27A | AIPVKRGATGETKVFTGNSNSPKSPTAGGC (SEQ ID NO: 164) |
| G28A | AIPVKRGATGETKVFTGNSNSPKSPTKAGC (SEQ ID NO: 165) |
| G29A | AIPVKRGATGETKVFTGNSNSPKSPTKGAC (SEQ ID NO: 166) |
| C30A | AIPVKRGATGETKVFTGNSNSPKSPTKGGA (SEQ ID NO: 167) |
| A1K | KIPVKRGATGETKVFTGNSNSPKSPTKGGC (SEQ ID NO: 168) |
| I2K | AKPVKRGATGETKVFTGNSNSPKSPTKGGC (SEQ ID NO: 169) |
| P3K | AIKVKRGATGETKVFTGNSNSPKSPTKGGC (SEQ ID NO: 170) |
| V4K | AIPKKRGATGETKVFTGNSNSPKSPTKGGC (SEQ ID NO: 171) |
| R6K | AIPVKKGATGETKVFTGNSNSPKSPTKGGC (SEQ ID NO: 172) |
| G7K | AIPVKRKATGETKVFTGNSNSPKSPTKGGC (SEQ ID NO: 173) |
| A8K | AIPVKRGKTGETKVFTGNSNSPKSPTKGGC (SEQ ID NO: 174) |
| T9K | AIPVKRGAKGETKVFTGNSNSPKSPTKGGC (SEQ ID NO: 175) |
| G10K | AIPVKRGATKETKVFTGNSNSPKSPTKGGC (SEQ ID NO: 176) |
| E11K | AIPVKRGATGKTKVFTGNSNSPKSPTKGGC (SEQ ID NO: 177) |
| T12K | AIPVKRGATGEKKVFTGNSNSPKSPTKGGC (SEQ ID NO: 178) |
| V14K | AIPVKRGATGETKKFTGNSNSPKSPTKGGC (SEQ ID NO: 179) |
| F15K | AIPVKRGATGETKVKTGNSNSPKSPTKGGC (SEQ ID NO: 180) |
| T16K | AIPVKRGATGETKVFKGNSNSPKSPTKGGC (SEQ ID NO: 181) |
| G17K | AIPVKRGATGETKVFTKNSNSPKSPTKGGC (SEQ ID NO: 182) |
| N18K | AIPVKRGATGETKVFTGKSNSPKSPTKGGC (SEQ ID NO: 183) |
| S19K | AIPVKRGATGETKVFTGNKNSPKSPTKGGC (SEQ ID NO: 184) |
| N20K | AIPVKRGATGETKVFTGNSKSPKSPTKGGC (SEQ ID NO: 185) |
| S21K | AIPVKRGATGETKVFTGNSNKPKSPTKGGC (SEQ ID NO: 186) |
| P22K | AIPVKRGATGETKVFTGNSNSKKSPTKGGC (SEQ ID NO: 187) |
| S24K | AIPVKRGATGETKVFTGNSNSPKKPTKGGC (SEQ ID NO: 188) |
| P25K | AIPVKRGATGETKVFTGNSNSPKSKTKGGC (SEQ ID NO: 189) |
| T26K | AIPVKRGATGETKVFTGNSNSPKSPKKGGC (SEQ ID NO: 190) |
| G28K | AIPVKRGATGETKVFTGNSNSPKSPTKKGC (SEQ ID NO: 191) |
| G29K | AIPVKRGATGETKVFTGNSNSPKSPTKGKC (SEQ ID NO: 192) |
| C30K | AIPVKRGATGETKVFTGNSNSPKSPTKGGK (SEQ ID NO: 193) |
| A1D | DIPVKRGATGETKVFTGNSNSPKSPTKGGC (SEQ ID NO: 194) |
| I2D | ADPVKRGATGETKVFTGNSNSPKSPTKGGC (SEQ ID NO: 195) |
| P3D | AIDVKRGATGETKVFTGNSNSPKSPTKGGC (SEQ ID NO: 196) |
| V4D | AIPDKRGATGETKVFTGNSNSPKSPTKGGC (SEQ ID NO: 197) |
| K5D | AIPVDRGATGETKVFTGNSNSPKSPTKGGC (SEQ ID NO: 198) |
| R6D | AIPVKDGATGETKVFTGNSNSPKSPTKGGC (SEQ ID NO: 199) |
| G7D | AIPVKRDATGETKVFTGNSNSPKSPTKGGC (SEQ ID NO: 200) |
| A8D | AIPVKRGDTGETKVFTGNSNSPKSPTKGGC (SEQ ID NO: 201) |
| T9D | AIPVKRGADGETKVFTGNSNSPKSPTKGGC (SEQ ID NO: 202) |
| G10D | AIPVKRGATDETKVFTGNSNSPKSPTKGGC (SEQ ID NO: 203) |
| E11D | AIPVKRGATGDTKVFTGNSNSPKSPTKGGC (SEQ ID NO: 204) |
| T12D | AIPVKRGATGEDKVFTGNSNSPKSPTKGGC (SEQ ID NO: 205) |
| K13D | AIPVKRGATGETDVFTGNSNSPKSPTKGGC (SEQ ID NO: 206) |
| V14D | AIPVKRGATGETKDFTGNSNSPKSPTKGGC (SEQ ID NO: 207) |
| F15D | AIPVKRGATGETKVDTGNSNSPKSPTKGGC (SEQ ID NO: 208) |
| T16D | AIPVKRGATGETKVFDGNSNSPKSPTKGGC (SEQ ID NO: 209) |
| G17D | AIPVKRGATGETKVFTDNSNSPKSPTKGGC (SEQ ID NO: 210) |
| N18D | AIPVKRGATGETKVFTGDSNSPKSPTKGGC (SEQ ID NO: 211) |
| S19D | AIPVKRGATGETKVFTGNDNSPKSPTKGGC (SEQ ID NO: 212) |
| N20D | AIPVKRGATGETKVFTGNSDSPKSPTKGGC (SEQ ID NO: 213) |
| S21D | AIPVKRGATGETKVFTGNSNDPKSPTKGGC (SEQ ID NO: 214) |
| P22D | AIPVKRGATGETKVFTGNSNSDKSPTKGGC (SEQ ID NO: 215) |
| K23D | AIPVKRGATGETKVFTGNSNSPDSPTKGGC (SEQ ID NO: 216) |
| S24D | AIPVKRGATGETKVFTGNSNSPKDPTKGGC (SEQ ID NO: 217) |
| P25D | AIPVKRGATGETKVFTGNSNSPKSDTKGGC (SEQ ID NO: 218) |
| T26D | AIPVKRGATGETKVFTGNSNSPKSPDKGGC (SEQ ID NO: 219) |
| K27D | AIPVKRGATGETKVFTGNSNSPKSPTDGGC (SEQ ID NO: 220) |
| G28D | AIPVKRGATGETKVFTGNSNSPKSPTKDGC (SEQ ID NO: 221) |
| G29D | AIPVKRGATGETKVFTGNSNSPKSPTKGDC (SEQ ID NO: 222) |
| C30D | AIPVKRGATGETKVFTGNSNSPKSPTKGGD (SEQ ID NO: 223) |
| A1E | EIPVKRGATGETKVFTGNSNSPKSPTKGGC (SEQ ID NO: 224) |
| I2E | AEPVKRGATGETKVFTGNSNSPKSPTKGGC (SEQ ID NO: 225) |
| P3E | AIEVKRGATGETKVFTGNSNSPKSPTKGGC (SEQ ID NO: 226) |
| V4E | AIPEKRGATGETKVFTGNSNSPKSPTKGGC (SEQ ID NO: 227) |
| K5E | AIPVERGATGETKVFTGNSNSPKSPTKGGC (SEQ ID NO: 228) |
| R6E | AIPVKEGATGETKVFTGNSNSPKSPTKGGC (SEQ ID NO: 229) |
| G7E | AIPVKREATGETKVFTGNSNSPKSPTKGGC (SEQ ID NO: 230) |
| A8E | AIPVKRGETGETKVFTGNSNSPKSPTKGGC (SEQ ID NO: 231) |
| T9E | AIPVKRGAEGETKVFTGNSNSPKSPTKGGC (SEQ ID NO: 232) |
| G10E | AIPVKRGATEETKVFTGNSNSPKSPTKGGC (SEQ ID NO: 233) |
| T12E | AIPVKRGATGEEKVFTGNSNSPKSPTKGGC (SEQ ID NO: 234) |
| K13E | AIPVKRGATGETEVFTGNSNSPKSPTKGGC (SEQ ID NO: 235) |
| V14E | AIPVKRGATGETKEFTGNSNSPKSPTKGGC (SEQ ID NO: 236) |
| F15E | AIPVKRGATGETKVETGNSNSPKSPTKGGC (SEQ ID NO: 237) |
| T16E | AIPVKRGATGETKVFEGNSNSPKSPTKGGC (SEQ ID NO: 238) |
| G17E | AIPVKRGATGETKVFTENSNSPKSPTKGGC (SEQ ID NO: 239) |
| N18E | AIPVKRGATGETKVFTGESNSPKSPTKGGC (SEQ ID NO: 240) |
| S19E | AIPVKRGATGETKVFTGNENSPKSPTKGGC (SEQ ID NO: 241) |
| N20E | AIPVKRGATGETKVFTGNSESPKSPTKGGC (SEQ ID NO: 242) |
| S21E | AIPVKRGATGETKVETGNSNEPKSPTKGGC (SEQ ID NO: 243) |
| P22E | AIPVKRGATGETKVFTGNSNSEKSPTKGGC (SEQ ID NO: 244) |
| K23E | AIPVKRGATGETKVFTGNSNSPESPTKGGC (SEQ ID NO: 245) |
| S24E | AIPVKRGATGETKVFTGNSNSPKEPTKGGC (SEQ ID NO: 246) |
| P25E | AIPVKRGATGETKVFTGNSNSPKSETKGGC (SEQ ID NO: 247) |
| T26E | AIPVKRGATGETKVFTGNSNSPKSPEKGGC (SEQ ID NO: 248) |
| K27E | AIPVKRGATGETKVFTGNSNSPKSPTEGGC (SEQ ID NO: 249) |
| G28E | AIPVKRGATGETKVFTGNSNSPKSPTKEGC (SEQ ID NO: 250) |
| G29E | AIPVKRGATGETKVFTGNSNSPKSPTKGEC (SEQ ID NO: 251) |
| C30E | AIPVKRGATGETKVFTGNSNSPKSPTKGGE (SEQ ID NO: 252) |

In some embodiments, a DddA variant comprises one or more amino acid substitutions relative to the amino acid sequence of SEQ ID NO: 139 (i.e., the C-terminal fragment of DddA split at position 1397). In some embodiments, a DddA variant comprises the point mutation D20. In some embodiments, a DddA variant comprises the point mutation E20. In some embodiments, a DddA variant comprises the point mutation K18. In some embodiments, a DddA variant comprises the point mutation K25. In some embodiments, a DddA variant comprises a C-terminal fragment comprising an amino acid sequence of any one of SEQ ID NOs: 140-252, or an amino acid sequence at least 80%, at least 85%, at least 90%, at least 95%, or at least 99% identical to the amino acid sequence of any one of SEQ ID NOs: 140-252.

In some embodiments, a DddA variant comprises a C-terminal fragment comprising an amino acid substitution at position N18. In certain embodiments, the amino acid substitution is an N18K substitution. In some embodiments, a DddA variant comprises a C-terminal fragment comprising an amino acid substitution at position P25. In certain embodiments, the amino acid substitution is a P25K substitution. In certain embodiments, the amino acid substitution is a P25A substitution. In certain embodiments, a DddA variant comprises a C-terminal fragment comprising an N18K substitution and a P25K substitution relative to the amino acid sequence of SEQ ID NO: 139. In certain embodiments, a DddA variant comprises a C-terminal fragment comprising an N18K substitution and a P25A substitution relative to the amino acid sequence of SEQ ID NO: 139.

In some embodiments, the DddA variants provided herein comprise truncations and/or extensions of either DddA fragment. As described further herein, it was hypothesized by the inventors that truncation of the N-terminal DddA fragment (G1397N) and/or truncation of the C-terminal DddA fragment (G1397C) would reduce the interaction interface between the two split DddA halves and weaken the spontaneous reassembly of DddA at off-target sites. In some embodiments, the N-terminal DddA fragment (G1397N) is truncated at its C-terminus (e.g., by deletion of 1, 2, 3, 4, 5, 6, 7, 8, 9, 10, or more than 10 amino acids). In some embodiments, the C-terminal DddA fragment (G1397C) is truncated at its N-terminus (e.g., by deletion of between 1-15 amino acids). In some embodiments, the C-terminal DddA fragment (G1397C) is truncated at its C-terminus by deletion of 1, 2, 3, 4, 5, 6, 7, 8, 9, 10, 11, 12, 13, 14, 15, or more than 15 amino acids. In particular, it was found that off-target editing was reduced by truncation of the N-terminal DddA fragment (G1397N) at its C-terminus by deletion of three amino acids without any observed lowering of on-target editing. This produced an even greater effect when combined with truncation of the C-terminal DddA fragment (G1397C) at its N-terminus by deletion of 5 amino acids.

Thus, in some embodiments, a DddA variant provided herein comprises a C-terminal fragment comprising an N-terminal amino acid truncation. In some embodiments, the C-terminal fragment comprises an N-terminal amino acid truncation of 1-15 amino acids in length (e.g., 1, 2, 3, 4, 5, 6, 7, 8, 9, 10, 11, 12, 13, 14, 15, or more than 15 amino acids in length). In some embodiments, a DddA variant comprises a C-terminal fragment comprising the amino acid sequence of any one of SEQ ID NOs: 253-267:

### N-Terminal Truncations of G1397C DddA Fragment:

| Truncation: | Sequence: |
|---|---|
| Canonical | AIPVKRGATGETKVFTGNSNSPKSPTKGGC (SEQ ID NO: 139) |
| NA1 | _IPVKRGATGETKVFTGNSNSPKSPTKGGC (SEQ ID NO: 253) |
| NA2 | __PVKRGATGETKVFTGNSNSPKSPTKGGC (SEQ ID NO: 254) |
| NA3 | ___VKRGATGETKVFTGNSNSPKSPTKGGC (SEQ ID NO: 255) |
| NA4 | ____KRGATGETKVFTGNSNSPKSPTKGGC (SEQ ID NO: 256) |
| NA5 | _____RGATGETKVFTGNSNSPKSPTKGGC (SEQ ID NO: 257) |
| NA6 | _______GATGETKVFTGNSNSPKSPTKGGC (SEQ ID NO: 258) |
| NA7 | ________ATGETKVFTGNSNSPKSPTKGGC (SEQ ID NO: 259) |
| NA8 | _________TGETKVFTGNSNSPKSPTKGGC (SEQ ID NO: 260) |
| NA9 | __________GETKVFTGNSNSPKSPTKGGC (SEQ ID NO: 261) |
| NA10 | ___________ETKVFTGNSNSPKSPTKGGC (SEQ ID NO: 262) |
| NA11 | ____________TKVFTGNSNSPKSPTKGGC (SEQ ID NO: 263) |
| NA12 | _____________KVFTGNSNSPKSPTKGGC (SEQ ID NO: 264) |
| NA13 | ______________VFTGNSNSPKSPTKGGC (SEQ ID NO: 265) |
| NA14 | _______________FTGNSNSPKSPTKGGC (SEQ ID NO: 266) |
| NA15 | ________________TGNSNSPKSPTKGGC (SEQ ID NO: 267) |

In some embodiments, a DddA variant provided herein comprises a C-terminal fragment comprising a C-terminal amino acid truncation. In some embodiments, the C-terminal fragment comprises a C-terminal amino acid truncation of 1-15 amino acids in length (e.g., 1, 2, 3, 4, 5, 6, 7, 8, 9, 10, 11, 12, 13, 14, 15, or more than 15 amino acids in length). In some embodiments, a DddA variant comprises a C-terminal fragment comprising the amino acid sequence of any one of SEQ ID NOs: 268-282:

### C-Terminal Truncations of G1397C DddA Fragment:

| Truncation: | Sequence: | |
|---|---|---|
| Canonical | AIPVKRGATGETKVFTGNSNSPKSPTKGGC | (SEQ ID NO: 139) |
| CA1 | AIPVKRGATGETKVFTGNSNSPKSPTKGG_ | (SEQ ID NO: 268) |
| CA2 | AIPVKRGATGETKVFTGNSNSPKSPTKG__ | (SEQ ID NO: 269) |
| CA3 | AIPVKRGATGETKVFTGNSNSPKSPTK___ | (SEQ ID NO: 270) |
| CA4 | AIPVKRGATGETKVFTGNSNSPKSPT____ | (SEQ ID NO: 271) |
| CA5 | AIPVKRGATGETKVFTGNSNSPKSP_____ | (SEQ ID NO: 272) |
| CA6 | AIPVKRGATGETKVFTGNSNSPKS______ | (SEQ ID NO: 273) |
| CA7 | AIPVKRGATGETKVFTGNSNSPK_______ | (SEQ ID NO: 274) |
| CA8 | AIPVKRGATGETKVFTGNSNSP________ | (SEQ ID NO: 275) |
| CA9 | AIPVKRGATGETKVFTGNSNS_________ | (SEQ ID NO: 276) |
| CA10 | AIPVKRGATGETKVFTGNSN__________ | (SEQ ID NO: 277) |
| CA11 | AIPVKRGATGETKVFTGNS___________ | (SEQ ID NO: 278) |
| CA12 | AIPVKRGATGETKVFTGN____________ | (SEQ ID NO: 279) |
| CA13 | AIPVKRGATGETKVFTG_____________ | (SEQ ID NO: 280) |
| CA14 | AIPVKRGATGETKVFT______________ | (SEQ ID NO: 281) |
| CA15 | AIPVKRGATGETKVF_______________ | (SEQ ID NO: 282) |

In some embodiments, a DddA variant provided herein comprises an N-terminal fragment comprising a C-terminal amino acid truncation. In some embodiments, the N-terminal fragment comprises a C-terminal amino acid truncation of 1-10 amino acids in length (e.g., 1, 2, 3, 4, 5, 6, 7, 8, 9, 10, or more than 10 amino acids in length). In certain embodiments, the N-terminal fragment comprises a C-terminal amino acid truncation of 3 amino acids in length. In some embodiments, a DddA variant comprises an N-terminal fragment comprising the amino acid sequence of any one of SEQ ID NOs: 284-293:

### C-Terminal Truncations of G1397N Fragment:

| Truncation: | Sequence: |
|---|---|
| Canonical | GSYALGPYQISAPQLPAYNGQTVGTFYYVNDAGGLESKVFSSGGPTPYPNYAN |
| | AGHVEGQSALFMRDNGISEGLVFHNNPEGTCGFCVNMTETLLPENAKMTVVPP |
| | EG (SEQ ID NO: 283) |
| CA1 | GSYALGPYQISAPQLPAYNGQTVGTFYYVNDAGGLESKVFSSGGPTPYPNYAN |
| | AGHVEGQSALFMRDNGISEGLVFHNNPEGTCGFCVNMTETLLPENAKMTVVPP |
| | E_ (SEQ ID NO: 284) |
| CA2 | GSYALGPYQI SAPQLPAYNGQTVGTFYYVNDAGGLESKVFSSGGPTPYPNYAN |
| | AGHVEGQSALFMRDNGISEGLVFHNNPEGTCGFCVNMTETLLPENAKMTVVPP__ |
| | (SEQ ID NO: 285) |
| CA3 | GSYALGPYQISAPQLPAYNGQTVGTFYYVNDAGGLESKVFSSGGPTPYPNYAN |
| | AGHVEGQSALFMRDNGISEGLVFHNNPEGTCGFCVNMTETLLPENAKMTVVP___ |
| | (SEQ ID NO: 286) |
| CA4 | GSYALGPYQISAPQLPAYNGQTVGTFYYVNDAGGLESKVFSSGGPTPYPNYAN |
| | AGHVEGQSALFMRDNGISEGLVFHNNPEGTCGFCVNMTETLLPENAKMTVV____ |
| | (SEQ ID NO: 287) |
| CA5 | GSYALGPYQISAPQLPAYNGQTVGTFYYVNDAGGLESKVFSSGGPTPYPNYAN |
| | AGHVEGQSALFMRDNGISEGLVFHNNPEGTCGFCVNMTETLLPENAKMTV_____ |
| | (SEQ ID NO: 288) |
| CA6 | GSYALGPYQISAPQLPAYNGQTVGTFYYVNDAGGLESKVFSSGGPTPYPNYAN |
| | AGHVEGQSALFMRDNGISEGLVFHNNPEGTCGFCVNMTETLLPENAKMT______ |
| | (SEQ ID NO: 289) |
| CA7 | GSYALGPYQISAPQLPAYNGQTVGTFYYVNDAGGLESKVFSSGGPTPYPNYAN |
| | AGHVEGQSALFMRDNGISEGLVFHNNPEGTCGFCVNMTETLLPENAKM_______ |
| | (SEQ ID NO: 290) |
| CA8 | GSYALGPYQISAPQLPAYNGQTVGTFYYVNDAGGLESKVFSSGGPTPYPNYAN |
| | AGHVEGQSALFMRDNGISEGLVFHNNPEGTCGFCVNMTETLLPENAK________ |
| | (SEQ ID NO: 291) |
| CA9 | GSYALGPYQISAPQLPAYNGQTVGTFYYVNDAGGLESKVFSSGGPTPYPNYAN |
| | AGHVEGQSALFMRDNGISEGLVFHNNPEGTCGFCVNMTETLLPENA_________ |
| | (SEQ ID NO: 292) |
| CA10 | GSYALGPYQISAPQLPAYNGQTVGTFYYVNDAGGLESKVFSSGGPTPYPNYAN |
| | AGHVEGQSALFMRDNGISEGLVFHNNPEGTCGFCVNMTETLLPEN__________ |
| | (SEQ ID NO: 293) |

In some embodiments, a DddA variant provided herein comprises an N-terminal fragment comprising a C-terminal amino acid extension. In some embodiments, the N-terminal fragment comprises a C-terminal amino acid extension of 1-15 amino acids in length (e.g., 1, 2, 3, 4, 5, 6, 7, 8, 9, 10, 11, 12, 13, 14, 15, or more than 15 amino acids in length). In some embodiments, a DddA variant comprises an N-terminal fragment comprising the amino acid sequence of any one of SEQ ID NOs: 294-308:

C-terminal extensions of G1397N fragment:

| Extension: | Sequence: |
|---|---|
| Canonical | GSYALGPYQISAPQLPAYNGQTVGTFYYVNDAGGLESKVFSSGGPTPYPNYA |
| | NAGHVEGQSALFMRDNGISEGLVFHNNPEGTCGFCVNMTETLLPENAKMTVV |
| | PPEG (SEQ ID NO: 283) |
| C + 1 | GSYALGPYQISAPQLPAYNGQTVGTFYYVNDAGGLESKVFSSGGPTPYPNYA |
| | NAGHVEGQSALFMRDNGISEGLVFHNNPEGTCGFCVNMTETLLPENAKMTVV |
| | PPEGA (SEQ ID NO: 294) |
| C + 2 | GSYALGPYQISAPQLPAYNGQTVGTFYYVNDAGGLESKVFSSGGPTPYPNYA |
| | NAGHVEGQSALFMRDNGISEGLVFHNNPEGTCGFCVNMTETLLPENAKMTVV |
| | PPEGAI (SEQ ID NO: 295) |
| C + 3 | GSYALGPYQISAPQLPAYNGQTVGTFYYVNDAGGLESKVFSSGGPTPYPNYA |
| | NAGHVEGQSALFMRDNGISEGLVFHNNPEGTCGFCVNMTETLLPENAKMTVV |
| | PPEGAIP (SEQ ID NO: 296) |
| C + 4 | GSYALGPYQISAPQLPAYNGQTVGTFYYVNDAGGLESKVFSSGGPTPYPNYA |
| | NAGHVEGQSALFMRDNGISEGLVFHNNPEGTCGFCVNMTETLLPENAKMTVV |
| | PPEGAIPV (SEQ ID NO: 297) |
| C + 5 | GSYALGPYQISAPQLPAYNGQTVGTFYYVNDAGGLESKVFSSGGPTPYPNYA |
| | NAGHVEGQSALFMRDNGISEGLVFHNNPEGTCGFCVNMTETLLPENAKMTVV |
| | PPEGAIPVK (SEQ ID NO: 298) |
| C + 6 | GSYALGPYQI SAPQLPAYNGQTVGTFYYVNDAGGLESKVFSSGGPTPYPNYA |
| | NAGHVEGQSALFMRDNGISEGLVFHNNPEGTCGFCVNMTETLLPENAKMTVV |
| | PPEGAIPVKR (SEQ ID NO: 299) |
| C + 7 | GSYALGPYQISAPQLPAYNGQTVGTFYYVNDAGGLESKVFSSGGPTPYPNYA |
| | NAGHVEGQSALFMRDNGISEGLVFHNNPEGTCGFCVNMTETLLPENAKMTVV |
| | PPEGAIPVKRG (SEQ ID NO: 300) |
| C + 8 | GSYALGPYQISAPQLPAYNGQTVGTFYYVNDAGGLESKVFSSGGPTPYPNYA |
| | NAGHVEGQSALFMRDNGISEGLVFHNNPEGTCGFCVNMTETLLPENAKMTVV |
| | PPEGAIPVKRGA (SEQ ID NO: 301) |
| C + 9 | GSYALGPYQISAPQLPAYNGQTVGTFYYVNDAGGLESKVFSSGGPTPYPNYA |
| | NAGHVEGQSALFMRDNGISEGLVFHNNPEGTCGFCVNMTETLLPENAKMTVV |
| | PPEGAIPVKRGAT (SEQ ID NO: 302) |
| C + 10 | GSYALGPYQISAPQLPAYNGQTVGTFYYVNDAGGLESKVFSSGGPTPYPNYA |
| | NAGHVEGQSALFMRDNGISEGLVFHNNPEGTCGFCVNMTETLLPENAKMTVV |
| | PPEGAIPVKRGATG (SEQ ID NO: 303) |
| C + 11 | GSYALGPYQISAPQLPAYNGQTVGTFYYVNDAGGLESKVFSSGGPTPYPNYA |
| | NAGHVEGQSALFMRDNGISEGLVFHNNPEGTCGFCVNMTETLLPENAKMTVV |
| | PPEGAIPVKRGATGE (SEQ ID NO: 304) |
| C + 12 | GSYALGPYQISAPQLPAYNGQTVGTFYYVNDAGGLESKVFSSGGPTPYPNYA |
| | NAGHVEGQSALFMRDNGISEGLVFHNNPEGTCGFCVNMTETLLPENAKMTVV |
| | PPEGAIPVKRGATGET (SEQ ID NO: 305) |
| C + 13 | GSYALGPYQISAPQLPAYNGQTVGTFYYVNDAGGLESKVFSSGGPTPYPNYA |
| | NAGHVEGQSALFMRDNGISEGLVFHNNPEGTCGFCVNMTETLLPENAKMTVV |
| | PPEGAIPVKRGATGETK (SEQ ID NO: 306) |
| C + 14 | GSYALGPYQISAPQLPAYNGQTVGTFYYVNDAGGLESKVFSSGGPTPYPNYA |
| | NAGHVEGQSALFMRDNGISEGLVFHNNPEGTCGFCVNMTETLLPENAKMTVV |
| | PPEGAIPVKRGATGETKV (SEQ ID NO: 307) |
| C + 15 | GSYALGPYQISAPQLPAYNGQTVGTFYYVNDAGGLESKVFSSGGPTPYPNYA |
| | NAGHVEGQSALFMRDNGISEGLVFHNNPEGTCGFCVNMTETLLPENAKMTVV |
| | PPEGAIPVKRGATGETKVF (SEQ ID NO: 308) |

In certain embodiments, a DddA variant further comprises a sequence of charged amino acid residues (for example, upstream of the DddA variant, e.g., in a linker joining the DddA variant to a pDNAbp such as a zinc finger domain-containing protein as described herein). As described further herein, it was hypothesized by the inventors that introduction of charged residues in the flexible linker between the ZF and the split DddA halves would introduce electrostatic repulsion that would weaken the spontaneous reassembly of DddA at off-target sites. In some embodiments, the charged sequence is GSGGGGSGDDDGS (SEQ ID NO: 319), GSGGGDDDDDDGS (SEQ ID NO: 320), GSDDDDDDDDDGS (SEQ ID NO: 321), GSGGGGSGGSDDD (SEQ ID NO: 316), GSGGGGSDDDDDD (SEQ ID NO: 317), GSGGDDDDDDDDD (SEQ ID NO: 318), GSGGGGSGEEEGS (SEQ ID NO: 313), GSGGGEEEEEEGS (SEQ ID NO: 314), GSEEEEEEEEEGS (SEQ ID NO: 315), GSGGGGSGGSEEE (SEQ ID NO: 310), GSGGGGSEEEEEE (SEQ ID NO: 311), or GSGGEEEEEEEEE (SEQ ID NO: 312). In some embodiments, the charged sequence is SGDDDGS (SEQ ID NO: 236), SGDDDDDDGS (SEQ ID NO: 327), SGDDDDDDDDDGS (SEQ ID NO: 328), DDDGS (SEQ ID NO: 323), DDDDDDGS (SEQ ID NO: 324), DDDDDDDDDGS (SEQ ID NO: 325), SGDDDGS (SEQ ID NO: 236), SGDDDDDDGS (SEQ ID NO: 327), SGDDDDDDDDDGS (SEQ ID NO: 328), DDDGS (SEQ ID NO: 323), DDDDDDGS (SEQ ID NO: 324), or DDDDDDDDDGS (SEQ ID NO: 325). In some embodiments, the sequence of charged amino acid residues comprises the amino acid sequence of any one of SEQ ID NOs: 309-334:

Charged residues upstream or downstream of split DddA to weaken binding affinity between split halves and lower off-target activity:

GSGGGGSGGSGGS (SEQ ID NO: 309)
GSGGGGSGGSEEE (SEQ ID NO: 310)
GSGGGGSEEEEEE (SEQ ID NO: 311)
GSGGEEEEEEEEE (SEQ ID NO: 312)
GSGGGGSGEEEGS (SEQ ID NO: 313)
GSGGGEEEEEEGS (SEQ ID NO: 314)
GSEEEEEEEEEGS (SEQ ID NO: 315)
GSGGGGSGGSDDD (SEQ ID NO: 316)
GSGGGGSDDDDDD (SEQ ID NO: 317)
GSGGDDDDDDDDD (SEQ ID NO: 318)
GSGGGGSGDDDGS (SEQ ID NO: 319)
GSGGGDDDDDDGS (SEQ ID NO: 320)
GSDDDDDDDDDGS (SEQ ID NO: 321)
SGGS (SEQ ID NO: 322)
DDDGS (SEQ ID NO: 323)
DDDDDDGS (SEQ ID NO: 324)
DDDDDDDDDGS (SEQ ID NO: 325)
SGDDDGS (SEQ ID NO: 326)
SGDDDDDDGS (SEQ ID NO: 327)
SGDDDDDDDDDGS (SEQ ID NO: 328)
EEEGS (SEQ ID NO: 329)
EEEEEEGS (SEQ ID NO: 330)
EEEEEEEEEGS (SEQ ID NO: 331)
SGEEEGS (SEQ ID NO: 332)
SGEEEEEEGS (SEQ ID NO: 333)
SGEEEEEEEEEGS (SEQ ID NO: 334)

In some embodiments, the sequence of charged amino acid residues may weaken the binding affinity of the first fragment and the second fragment of the DddA variant to one another.

In some embodiments, a DddA variant further comprises a catalytically dead second DddA fragment fused to the first DddA fragment. As described further herein, DddA can be catalytically inactivated by introduction of an E1347A mutation. In the G1397-split architecture, this mutation lies in the N-terminal DddA fragment (G1397N). It was hypothesized by the inventors that by fusing a catalytically-inactivated N-terminal DddA fragment (G1397N) adjacent to the C-terminal DddA fragment (G1397C), the catalytically-inactivated fragment would compete for reassembly and would weaken the spontaneous reassembly of catalytically-active DddA at off-target sites. Thus, the present disclosure provides ZF-DdCBE constructs in which a catalytically-inactivated N-terminal DddA fragment (G1397N) was fused downstream of the C-terminal DddA fragment (G1397C), either before or after the UGI, using flexible linkers of different lengths. In some embodiments, the catalytically dead second DddA fragment comprises the amino acid sequence of SEQ ID NO: 335, or an amino acid sequence that is at least 80%, at least 85%, at least 90%, at least 95%, or at least 99% identical to the amino acid sequence of SEQ ID NO: 335:

### Fusion of “Dead” DddA N-Terminal Domain to C-Terminal DddA Fragment to Reduce Off-Target Activity:

| | |
|---|---|
| Canonical | GSYALGPYQISAPQLPAYNGQTVGTFYYVNDAGGLESKVF |
| | SSGGPTPYPNYANAGHVEGQSALFMRDNGISEGLVFHNN |
| | PEGTCGFCVNMTETLLPENAKMTVVPPEG |
| | (SEQ ID NO: 283) |
| Dead | GSYALGPYQISAPQLPAYNGQTVGTFYYVNDAGGLESKVF |
| (E1347A) | SSGGPTPYPNYANAGHVAGQSALFMRDNGISEGLVFHNNP |
| | EGTCGFCVNMTETLLPENAKMTVVPPEG |
| | (SEQ ID NO: 335) |

The changes made in each of the DddA variants provided herein relative to wild type DddA may be made in any combination with one another. In some embodiments, combining two or more of the point mutations, truncation, extensions, etc. described herein will result in a DddA variant with even more increased on-target editing activity and/or decreased off-target editing activity relative to a DddA variant comprising only a single point mutation, truncation, extension, etc. Mutants comprising an N18K mutation, N18K and P25A mutations, and N18K and P25K mutations showed particularly promising increases in activity. Variants comprising a truncation of the three C-terminal amino acids of the N-terminal DddA fragment also showed particularly promising increases in activity, especially in combination with N18K and/or P25A or P25K mutations. Thus, in some embodiments, a DddA variant comprises a C-terminal fragment comprising amino acid substitutions at positions N18 and P25 and an N-terminal fragment comprising a C-terminal amino acid truncation of 3 amino acids in length. In certain embodiments, the C-terminal fragment comprises the amino acid substitutions N18K and P25A, and the N-terminal fragment comprises a C-terminal amino acid truncation of 3 amino acids in length. In certain embodiments, the C-terminal fragment comprises the amino acid substitutions N18K and P25K, and the N-terminal fragment comprises a C-terminal amino acid truncation of 3 amino acids in length.

Any of the point mutations, amino acid truncations, extensions, etc. described herein can also be made at corresponding positions in other DddA enzymes and homologs. In various embodiments, the following exemplary DddA enzymes, or variants thereof, can be used to create additional DddA variants comprising the point mutations, amino acid truncations, extensions, etc. described herein, or a sequence (amino acid or nucleotide as the case may be) having at least 70%, 75%, 80%, 85%, 90%, 95%, or 99% sequence identity with any one of the following DddA sequences:

| DddA | |
|---|---|
| Description | DddA amino acid and/or nucleotide sequence |
| DddA | >ATF83755.1 hypothetical protein CO712_00910 |
| homolog in | [Burkholderia gladioli pv. Gladioli] |
| Burkholderia | MYEAARVTDPIEHTSALAGFLVGAVLGIALIAAVAFATFTCGFGVALLAGM |
| gladioli | AAGIGAQVLLSLGESIGKMFSSQSGAITLGSPNVYVNGKQAAYATLSSVTCS |
| PROTEIN | KHNPTPLVAQGSTNIFINGKPAARKDDKITCGAAISDGSHDTYFHGGIQTCLP |
| | IDDEVPPWLRTATDWAFALAGLVGGLGGLLKEAGGLSHAVMPCAAKFIGG |
| | YVLGEAASRYVIGPAINSAIGGMFGNPVDVTTGRKILPAESETDYVVPSPMP |
| | VAIRRFYSSDLDYVGTLGRGWVLPWELRLHARDGRLWYTDAQGRESGFPIL |
| | KPGQAAFSEADQRYLTCTPDGRYILHDVGETYYDFGRYEPGSGRIGWVRRIE |
| | DQAGQWCQFERDSRGRVREIQTCGGLLAVLDYEPEHERLAEVSLVSGDQRR |
| | LVVAYGYDENGQMASVTDANGAVVRRFTYADGRMTSHSNALGFTSGYTW |
| | KVIDGTPRVVATHTSEGEAWAFEYDIEGRRTHVRHADGRHAQWRYDAQFQ |
| | IVEYLDFDGRRYGLKYNAAGMPVMLTLPGERTVMFEYDDAGRIVAETDPLG |
| | RTTKTRYDGNSMRPVEIILPDGSAWHAEYDRQGRLLVTRDPLDRENRYEYP |
| | EALSALPVAHVDALGGRKTFEWNRLGELVAYTDCSGKTTRNFFDAFGLPLA |
| | RENALGHRVSFDLRPTGETRRVTYPDGSSESYEYDAAGLMIRHIGLGGRMQ |
| | TLQRNARGQLVEAVDPAGRRTRYHYDAEGRLRELQQAHARYAFAYSAGGR |
| | LVSETRPDGVLRRFEYGEAGDLAALEIVGTADDCAPNDRPVRAIRFERDRM |
| | GNLCVQHTPTEVTRYERDAGGRLLEVASVPTAAGLALGIAPDTLTFEYDKA |
| | GRLSAEHGANGSVQYTLDALDNVLKLALPHEQTLQMLRYGSGHVHQIRHG |
| | DQVVSDFERDDLHRELTRTQGPLTERTAYDLLGRKIWQSAGFQPDALARGQ |
| | GQLWRNYGYDAAGELVESHDSLRGSTQFSYDPAGYLTQRVNTADRQLESF |
| | AWDAAGNLLDDAQRSSRGYVEGNRLRMWQNLRFDYDAFGNLATKLRGAN |
| | QRQQFTYDGQDRLVAVRTQGARGVVETRFAYDPLGRRIAKTDRTLDVRGV |
| | TLREETKRFVWEGLRLAQEVRDTGVSSYVYSPDAPYMPAARVDAVKAEAL |
| | ANAAIDKARQATRIYHFHTDVSGAPQEATNEAGDIVWAGQYSAWGKVAPN |
| | QHAPARIDQPLRYAGQYADDSTELHYNTFRFYDPDVGRFINQDPIGLMGGL |
| | NLYQYAPNSIAWTDWWGLAGSYTLGSYQISAPQLPAYNGQTVGTFYYVND |
| | AGGLESRTFSSGGPTPYPNYANAGHVEGQSALFMRDNGISDGLVFHNNPEG |
| | TCGFCVNMTETLLPENSKLTVVPPEGSIPVKRGATGETRTFTGNSKSPKSPVK |
| | GGC (SEQ ID NO: 361) |
| DddA | >CO712_00910 NZ_CP023522.1:185368-189645 |
| homolog in | Burkholderia gladioli pv. Gladioli strain |
| Burkholderia | FDAARGOS_389 chromosome 1, complete sequence |
| gladioli | GTGTACGAAGCGGCCCGCGTCACGGATCCGATCGAGCACACCAGCGCGC |
| DNA | TGGCCGGCTTCCTGGTGGGCGCCGTGCTCGGTATCGCCCTGATTGCTGCC |
| | GTGGCGTTCGCCACGTTCACCTGCGGCTTCGGCGTGGCACTGCTGGCCGG |
| | CATGGCGGCCGGCATCGGCGCGCAGGTGCTGTTGTCGTTAGGGGAATCG |
| | ATCGGGAAGATGTTCAGTTCGCAATCCGGCGCGATCACGCTCGGCTCGCC |
| | GAACGTCTACGTGAACGGCAAGCAGGCCGCCTACGCCACGCTCAGCAGC |
| | GTGACGTGCAGCAAGCACAACCCGACGCCGCTCGTCGCGCAGGGCTCCA |
| | CCAACATCTTCATCAACGGCAAGCCGGCCGCGCGCAAGGACGACAAGAT |
| | CACCTGCGGCGCGGCCATCTCGGACGGCTCGCACGACACCTACTTCCACG |
| | GAGGCATCCAGACCTGCCTGCCGATCGACGACGAAGTGCCGCCGTGGCT |
| | GCGCACCGCCACCGACTGGGCGTTCGCGCTGGCCGGGCTGGTGGGCGGG |
| | CTCGGCGGCCTACTCAAGGAAGCGGGCGGGCTGTCGCACGCGGTGATGC |
| | CGTGCGCGGCGAAGTTCATCGGCGGCTACGTGCTCGGCGAGGCGGCGAG |
| | CCGCTACGTGATCGGCCCGGCCATCAACAGCGCGATCGGCGGGATGTTC |
| | GGCAACCCGGTAGACGTCACCACTGGGCGCAAGATCCTCCCTGCCGAAT |
| | CGGAAACCGATTACGTCGTGCCCAGCCCGATGCCGGTGGCGATCCGGCG |
| | CTTCTATTCGAGCGACCTCGATTACGTCGGCACGCTTGGGCGCGGCTGGG |
| | TGCTGCCGTGGGAGCTGCGCCTGCACGCGCGTGACGGTCGGCTCTGGTAC |
| | ACCGACGCGCAGGGGCGCGAGAGCGGCTTCCCGATCCTGAAACCGGGCC |
| | AGGCCGCGTTCAGCGAGGCCGATCAGCGCTATCTGACCTGCACGCCGGA |
| | TGGCCGCTACATCCTCCACGACGTCGGCGAAACCTATTACGACTTCGGCC |
| | GCTACGAGCCGGGCTCGGGCCGCATCGGCTGGGTGCGCCGGATCGAGGA |
| | TCAGGCCGGCCAGTGGTGCCAGTTCGAGCGCGACAGCCGTGGCCGCGTG |
| | CGTGAAATCCAGACCTGCGGCGGCTTGCTGGCCGTGCTCGATTACGAGCC |
| | GGAGCACGAGCGGCTCGCCGAGGTGTCGCTCGTCAGCGGCGATCAGCGC |
| | CGCCTCGTCGTGGCCTACGGCTACGACGAAAACGGCCAGATGGCCTCCG |
| | TGACCGACGCGAACGGCGCGGTGGTGCGCCGCTTCACCTATGCCGACGG |
| | GCGCATGACGAGCCATTCGAACGCGCTCGGTTTCACGTCGGGCTATACGT |
| | GGAAGGTCATCGACGGCACGCCGCGAGTGGTCGCCACCCACACCAGCGA |
| | GGGCGAGGCCTGGGCGTTCGAGTACGACATCGAAGGCCGCCGCACCCAT |
| | GTGCGGCATGCCGACGGCCGCCACGCGCAATGGCGCTACGACGCGCAAT |
| | TCCAGATCGTCGAGTACCTCGATTTCGACGGCCGTCGCTACGGGCTCAAG |
| | TACAACGCTGCCGGCATGCCCGTGATGCTGACGCTGCCCGGCGAACGAA |
| | CCGTGATGTTCGAGTACGACGACGCCGGCCGCATCGTCGCCGAAACCGA |
| | TCCCCTCGGCCGCACCACGAAAACGCGCTACGACGGCAACAGCATGCGG |
| | CCCGTCGAGATCATCTTGCCCGACGGCAGCGCCTGGCACGCCGAATACG |
| | ACCGGCAGGGCCGGCTGCTCGTCACCCGTGATCCGCTCGACCGGGAGAA |
| | TCGCTACGAATATCCGGAGGCACTGAGCGCGCTCCCGGTGGCGCATGTC |
| | GATGCGCTGGGCGGGCGCAAGACGTTCGAGTGGAACCGGCTCGGCGAGC |
| | TGGTGGCCTACACCGATTGCTCGGGCAAGACCACGCGCAATTTTTTCGAT |
| | GCATTCGGCCTGCCGCTCGCGCGCGAGAACGCGCTCGGGCACCGCGTGT |
| | CGTTCGATCTGCGCCCGACCGGCGAGACGCGCCGCGTCACCTATCCCGAC |
| | GGCAGTTCCGAAAGCTACGAATACGACGCCGCCGGGCTGATGATCCGGC |
| | ACATCGGGCTGGGCGGCCGGATGCAGACGTTGCAGCGCAATGCGCGCGG |
| | GCAACTCGTCGAGGCGGTCGATCCGGCCGGGCGGCGAACCCGCTACCAC |
| | TACGACGCCGAAGGGCGGCTGCGCGAGCTGCAACAGGCCCACGCGCGCT |
| | ACGCATTCGCGTACAGCGCAGGCGGGCGGCTTGTCAGCGAAACGCGGCC |
| | CGACGGCGTGCTGCGCCGCTTCGAATACGGCGAGGCCGGCGATCTGGCG |
| | GCGCTCGAGATCGTCGGAACGGCCGATGATTGCGCTCCAAACGATCGCC |
| | CGGTTCGCGCGATCCGCTTCGAGCGCGACCGGATGGGTAACCTGTGCGTG |
| | CAGCACACGCCTACCGAGGTGACGCGCTACGAGCGCGACGCCGGCGGCC |
| | GCCTGCTCGAAGTCGCGAGCGTGCCGACCGCGGCCGGACTGGCGCTCGG |
| | CATCGCGCCCGACACGCTGACCTTCGAATACGACAAGGCCGGGCGGCTG |
| | AGCGCCGAACACGGCGCGAACGGCAGCGTCCAGTACACGCTCGACGCGC |
| | TCGACAACGTGTTGAAGCTCGCCTTGCCGCACGAACAGACGCTGCAGAT |
| | GCTGCGCTACGGCTCGGGGCACGTGCACCAGATTCGCCACGGCGACCAG |
| | GTCGTCAGCGATTTCGAGCGCGACGACCTGCATCGCGAGTTGACGCGCA |
| | CGCAGGGCCCCCTGACCGAGCGGACCGCCTACGACCTGCTGGGCCGCAA |
| | GATCTGGCAATCAGCCGGCTTCCAGCCCGACGCGCTTGCGCGTGGGCAG |
| | GGCCAGCTGTGGCGCAACTACGGCTACGACGCCGCCGGGGAACTGGTCG |
| | AGAGCCACGACAGCCTGCGCGGCAGCACGCAGTTCAGCTACGATCCGGC |
| | CGGCTATCTGACGCAGCGCGTGAACACCGCCGACCGGCAGCTCGAATCG |
| | TTCGCCTGGGACGCCGCCGGCAACCTGCTCGACGATGCGCAACGCAGCA |
| | GCCGCGGCTATGTCGAGGGCAACCGGCTGCGCATGTGGCAGAACCTGCG |
| | CTTCGACTACGACGCGTTCGGCAATCTCGCGACCAAGCTGCGCGGCGCG |
| | AATCAGCGCCAGCAGTTCACGTACGATGGGCAGGATCGGCTCGTGGCCG |
| | TGCGCACGCAGGGCGCGCGCGGCGTGGTGGAGACGCGTTTCGCCTACGA |
| | TCCGCTCGGGCGGCGCATCGCCAAGACCGATAGGACACTCGACGTGCGC |
| | GGCGTAACGCTGCGCGAGGAAACGAAGCGGTTCGTATGGGAAGGGCTGC |
| | GGCTCGCGCAGGAGGTGCGCGACACCGGCGTGAGCAGCTACGTGTACAG |
| | CCCGGATGCGCCTTACATGCCCGCGGCGCGGGTCGATGCGGTGAAAGCC |
| | GAAGCGCTCGCAAACGCCGCGATCGACAAGGCCAGACAGGCGACGCGG |
| | ATCTATCACTTTCATACCGATGTGTCGGGCGCACCGCAAGAAGCGACGA |
| | ACGAGGCCGGCGACATTGTTTGGGCCGGCCAATACTCAGCCTGGGGCAA |
| | GGTGGCGCCGAACCAGCATGCCCCAGCCCGGATCGATCAGCCGCTCCGC |
| | TACGCCGGACAATATGCCGATGACAGTACCGAGCTGCACTACAACACGT |
| | TTCGTTTCTACGATCCGGATGTCGGCCGGTTTATCAATCAGGATCCAATC |
| | GGGTTGATGGGGGGGCTGAATCTTTACCAATATGCACCCAACTCAATCGC |
| | GTGGACCGACTGGTGGGGGCTGGCCGGCAGCTATACGCTCGGTTCCTATC |
| | AAATTTCTGCTCCTCAACTTCCCGCCTACAATGGGCAGACTGTTGGGACC |
| | TTCTACTATGTAAACGACGCGGGCGGGCTCGAATCGAGGACATTCTCTTC |
| | TGGAGGGCCGACCCCTTATCCAAATTATGCCAATGCCGGGCACGTGGAA |
| | GGCCAGTCCGCACTGTTCATGAGGGATAACGGAATTTCAGACGGACTGG |
| | TTTTCCACAACAACCCTGAGGGTACTTGCGGATTCTGCGTCAATATGACC |
| | GAAACGCTTTTGCCTGAAAATTCCAAACTTACCGTCGTTCCGCCCGAGGG |
| | CTCGATTCCGGTCAAGCGGGGCGCGACGGGCGAAACGAGAACATTTACA |
| | GGGAACAGCAAGTCTCCGAAGTCCCCTGTCAAAGGAGGATGTTGA (SEQ |
| | ID NO: 362) |
| DddA | >AJY63123.1 RHS repeat-associated core domain pro- |
| homolog in | tein [Burkholderia glumae LMG 2196 = ATCC 33617] |
| Burkholderia | MYEAARVTDPIEHTSALTGFLVGAVLGIALIAAVAFATFTCGFGVALLAGMA |
| glumae LMG | AGIGAQVLLSLGESIGKMFSSQSGAITLGSPNVYVNGKPTAYAMLSSVTCSK |
| 2196 | HNPTPLVAQGSTNIFINGKPAARKDDKITCGATISDGSHDTYFHGGTQTCLPI |
| PROTEIN | DDEVPPWLRTATDWAFALAGLVGGLGGLLKEAGGLSRAVMPCAAKFIGGY |
| | VLGEAASRYVVGPAINSAIGGMFGNPVDVTTGRKILLAESETDYVVPSPMPV |
| | AIRRFYSSDLDYVGTLGRGWVLPWELRLHARDGRLWYTDAQGRESGFPML |
| | QPGHAAFSEADQRYLTCTPDGRYILHDLGETYYDFGHYEPGSGRIGWVRRIE |
| | DQAGQWCQFERDSRGRVREIQTCGGLLAVLDYEPEHGRLAGVSLVSGDQR |
| | RLVVAYGYDEHGQMASVTDANGALVRRFTYADGRMTSHSNALGFTSGYT |
| | WQAVGGAPRVVATHTSEGEAWAFEYDIEGRRTHVRHADGRHAQWRYDAQ |
| | FQIVEYLDFDGRRYGLKYNDAGMPVMLTLPGERTVTFEYDDAGRIVAETDP |
| | LGRTTKTRYDGNSRRPVEIIAPDGSAWHAEYDRQGRLLATRDPLDRENRYE |
| | YPKALSALPIAHVDALGGRKTFEWNRLGELVAYTDCSGKTTRNFYDAFGLP |
| | LARENALGHRVTFDLRPTGEARRVTYPDGSTESYEYDAAGLMIRHVGLGGR |
| | TQIALRNARGQIVEAVDPAGRRTCYRYDAEGRLRELQQGHARYAFTYSAGG |
| | RLTSETRPDGVRRRFEYGEAGDLAALDIVGAADDATANDRPVRTIRFERDR |
| | MGNLCAQHTPTEVTRYTRDTGGRLLEVACVPTAAGLALGIAPDTLTFEYDK |
| | AGRLSAEHGANGSVRYTLDALDNVMKLALPHEQTLQMLRYGSGHVHQIRC |
| | GDQVVSDFERDDLHRELTRTQGRLTERTAYDLLGRKIWQSAGFQPDALARG |
| | QGQVWRNYGYDAAGELAESHDSLRGSTQFSYDPAGYLTQRVNTADRQLES |
| | FAWDAAGNLLDDAQRRSRGYVEGNRLRMWQNLRFEYDPFGNLATKLRGA |
| | NQRQQFTYDGQDRLVAVRTQDARGVVETRFAYDPLGRRIAKTDIVRDARG |
| | VALREETKRFVWEGLRLAQEVRDTGVSSYVYSPDAPYTPAARVDAVLAEA |
| | MAAAAIEQARQATRIYHFHTDVSGAPQEATNEAGDIVWAGQYSAWGKVAP |
| | NQHAPARIDOPLRYAGQYADDSTELHYNTFRFYDPDVGRFINQDPIGLMGG |
| | LNLYQYAPNSIAWTDWWGLAGSYTLGSYQISAPQLPAYNGQTVGTFYYVN |
| | GAGGLESRTFSSGGPTPYPNYANAGHVEGQSALFMRDNGISDGLVFHNNPE |
| | GTCGFCVNMTETLLPENSKLTVVPPEGAIPVKRGATGETRTFTGNSKSPKSPV |
| | KGEC (SEQ ID NO: 363) |
| DddA | >KS03_3390 CP009434.1:65330-69607 Burkholderia |
| homolog in | glumae LMG 2196 = ATCC 33617 chromosome II, |
| Burkholderia | complete sequence |
| glumae LMG | GTGTACGAAGCGGCCCGCGTCACCGACCCGATCGAACACACCAGCGCGC |
| 2196 | TGACCGGCTTTCTGGTGGGCGCCGTGCTCGGCATTGCCCTGATCGCCGCG |
| DNA | GTGGCGTTCGCCACCTTCACCTGCGGCTTCGGCGTGGCGCTGCTGGCCGG |
| | CATGGCCGCCGGCATCGGCGCGCAGGTGCTGTTGTCGTTAGGAGAATCG |
| | ATCGGGAAGATGTTCAGTTCGCAATCCGGCGCGATCACGCTCGGCTCGCC |
| | GAACGTCTATGTGAACGGCAAGCCGACCGCCTACGCCATGCTCAGCAGC |
| | GTGACGTGCAGCAAGCACAACCCGACGCCGCTCGTCGCGCAGGGGTCCA |
| | CCAACATCTTCATCAACGGCAAGCCGGCCGCCCGCAAGGACGACAAGAT |
| | CACCTGCGGCGCGACCATCTCCGACGGCTCGCACGACACCTATTTCCACG |
| | GCGGCACCCAGACCTGCCTGCCGATCGACGACGAAGTGCCGCCGTGGCT |
| | GCGCACCGCCACCGACTGGGCGTTCGCGCTGGCCGGGCTGGTGGGCGGG |
| | CTCGGCGGCCTGCTCAAGGAAGCGGGCGGGCTGTCGCGCGCGGTGATGC |
| | CGTGCGCGGCGAAGTTCATCGGCGGCTACGTGCTCGGCGAGGCGGCGAG |
| | CCGCTACGTGGTCGGCCCGGCCATCAACAGCGCGATCGGCGGGATGTTC |
| | GGCAACCCGGTGGACGTCACCACCGGGCGCAAGATCCTGCTGGCGGAAT |
| | CGGAAACCGATTACGTGGTGCCCAGCCCGATGCCGGTGGCGATCCGGCG |
| | CTTCTATTCGAGCGACCTCGACTACGTCGGCACGCTCGGGCGCGGCTGGG |
| | TGCTGCCGTGGGAACTGCGGCTGCACGCGCGCGACGGGCGGCTCTGGTA |
| | CACCGACGCGCAGGGGCGCGAGAGCGGCTTCCCGATGCTCCAGCCGGGC |
| | CATGCCGCGTTCAGCGAGGCCGACCAGCGCTATCTGACCTGCACCCCGG |
| | ATGGCCGCTACATCCTGCACGACCTCGGCGAAACCTATTACGACTTCGGC |
| | CACTACGAGCCGGGCTCGGGCCGCATCGGCTGGGTGCGCCGCATCGAGG |
| | ATCAGGCCGGCCAGTGGTGCCAGTTCGAGCGCGACAGCCGCGGCCGCGT |
| | GCGCGAAATCCAGACCTGCGGCGGCTTGCTGGCCGTGCTCGATTACGAG |
| | CCGGAACACGGGCGGCTCGCCGGGGTGTCGCTCGTCAGCGGGGATCAGC |
| | GCCGCCTCGTGGTGGCTTACGGCTATGACGAGCACGGCCAGATGGCGTC |
| | CGTGACCGATGCGAACGGCGCGCTGGTGCGCCGCTTCACCTATGCCGAC |
| | GGGCGCATGACGAGCCATTCGAACGCGCTCGGCTTCACGTCGGGCTATA |
| | CGTGGCAAGCCGTCGGCGGCGCGCCGCGGGTGGTTGCCACCCACACCAG |
| | CGAGGGCGAGGCCTGGGCCTTCGAGTACGACATTGAAGGACGCCGCACC |
| | CACGTGCGTCACGCCGACGGCCGCCACGCGCAATGGCGCTACGACGCGC |
| | AATTCCAGATCGTCGAGTACCTCGATTTCGACGGCCGGCGCTACGGGCTC |
| | AAGTACAACGACGCCGGCATGCCCGTGATGCTGACGCTGCCCGGCGAAC |
| | GGACCGTGACGTTCGAGTACGACGATGCCGGCCGCATCGTCGCCGAAAC |
| | CGATCCACTCGGCCGCACCACGAAAACGCGCTACGACGGCAACAGCAGG |
| | CGGCCCGTCGAGATCATCGCGCCCGACGGCAGCGCCTGGCACGCCGAAT |
| | ACGACCGGCAAGGCCGGCTGCTCGCCACCCGCGATCCGCTCGACCGGGA |
| | AAACCGCTACGAATACCCGAAGGCGCTCAGCGCGCTGCCGATCGCGCAC |
| | GTCGATGCGCTGGGCGGGCGCAAGACGTTCGAGTGGAACCGGCTCGGCG |
| | AGCTGGTGGCCTATACCGATTGCTCGGGCAAGACCACACGCAATTTTTAC |
| | GACGCATTCGGTCTGCCGCTCGCGCGCGAGAACGCGCTCGGCCACCGCG |
| | TGACGTTCGACCTGCGCCCGACCGGCGAGGCGCGGCGCGTCACCTATCCC |
| | GACGGCAGTACAGAAAGCTACGAATACGACGCCGCCGGGCTGATGATCC |
| | GGCACGTCGGGCTGGGCGGCCGGACGCAGATTGCGCTGCGCAACGCGCG |
| | TGGGCAGATCGTGGAGGCGGTCGATCCGGCCGGACGGCGCACCTGCTAC |
| | CGCTACGACGCCGAGGGGCGGCTGCGCGAGCTGCAACAGGGGCACGCGC |
| | GTTACGCGTTCACCTACAGCGCGGGCGGGCGGCTCACCAGCGAAACCCG |
| | GCCCGACGGCGTGCGGCGCCGCTTCGAATACGGCGAGGCCGGCGATCTG |
| | GCGGCGCTCGACATCGTCGGCGCGGCCGACGACGCCACGGCGAACGATC |
| | GTCCGGTTCGCACCATCCGCTTCGAGCGCGACCGCATGGGCAATCTGTGC |
| | GCGCAGCACACGCCCACCGAGGTGACGCGCTACACGCGCGACACCGGCG |
| | GCCGCCTGCTCGAAGTCGCATGCGTGCCGACCGCGGCCGGGCTGGCGCT |
| | CGGCATCGCGCCCGACACGCTGACCTTCGAATACGACAAGGCCGGGCGG |
| | CTGAGTGCCGAACACGGCGCGAACGGCAGCGTCCGATACACGCTCGACG |
| | CGCTCGACAACGTGATGAAGCTCGCCCTGCCGCACGAGCAGACGCTGCA |
| | GATGCTGCGCTACGGCTCGGGGCACGTGCATCAGATCCGCTGCGGCGAC |
| | CAGGTGGTCAGCGATTTCGAGCGCGACGACCTGCATCGCGAGCTGACGC |
| | GCACTCAGGGCCGCCTGACCGAGCGTACCGCCTACGACCTGCTGGGCCG |
| | CAAGATCTGGCAATCGGCCGGCTTCCAGCCCGACGCGCTTGCGCGCGGG |
| | CAGGGCCAGGTGTGGCGCAACTACGGCTACGACGCCGCCGGCGAACTGG |
| | CCGAGAGCCACGATAGCCTGCGCGGCAGCACGCAGTTCAGCTACGATCC |
| | GGCCGGCTATCTGACGCAGCGCGTCAATACCGCCGACCGGCAGCTCGAA |
| | TCGTTCGCCTGGGATGCCGCCGGCAACCTGCTCGACGATGCGCAGCGCCG |
| | CAGCCGCGGTTATGTCGAGGGCAACCGGCTGCGCATGTGGCAGAACCTG |
| | CGCTTCGAATACGACCCGTTCGGCAATCTCGCGACCAAGCTGCGCGGCGC |
| | GAACCAGCGCCAGCAGTTCACTTACGACGGGCAGGATCGGCTCGTGGCG |
| | GTGCGCACGCAGGACGCGCGCGGCGTGGTGGAGACGCGTTTCGCCTACG |
| | ATCCGCTGGGGCGGCGCATCGCCAAGACGGATATTGTGCGCGACGCGCG |
| | CGGCGTAGCGCTGCGCGAGGAAACGAAGCGGTTCGTGTGGGAGGGGCTG |
| | CGGCTCGCGCAGGAGGTGCGCGACACGGGCGTGAGCAGCTACGTGTACA |
| | GCCCGGACGCGCCCTATACGCCCGCGGCGCGCGTGGATGCCGTGCTGGC |
| | CGAGGCCATGGCCGCCGCTGCCATCGAGCAGGCCAGACAGGCGACGCGG |
| | ATCTATCACTTTCATACCGATGTGTCGGGCGCACCGCAAGAAGCGACGA |
| | ACGAGGCTGGCGACATTGTTTGGGCCGGCCAATACTCAGCCTGGGGCAA |
| | GGTGGCGCCGAACCAGCATGCCCCCGCCCGGATCGATCAGCCGCTCCGC |
| | TACGCCGGACAATATGCCGACGACAGTACCGAGCTGCACTACAACACGT |
| | TTCGTTTCTACGATCCGGACGTCGGCCGGTTTATCAATCAGGATCCAATC |
| | GGGTTGATGGGGGGGCTGAATCTTTACCAATATGCACCCAACTCGATCGC |
| | ATGGACCGACTGGTGGGGGCTGGCCGGCAGCTATACGCTCGGTTCCTATC |
| | AAATTTCTGCGCCTCAACTTCCGGCCTACAATGGACAGACTGTTGGGACC |
| | TTCTACTACGTGAACGGCGCGGGCGGGCTCGAATCGAGGACATTCTCTTC |
| | CGGAGGGCCGACCCCTTATCCAAATTATGCCAATGCCGGGCACGTGGAG |
| | GGCCAGTCCGCGCTGTTCATGAGGGATAACGGAATTTCAGACGGACTGG |
| | TTTTCCACAACAACCCTGAGGGCACTTGCGGATTCTGCGTTAATATGACC |
| | GAAACGCTTTTGCCTGAAAATTCCAAACTTACCGTCGTTCCGCCCGAGGG |
| | CGCGATCCCGGTCAAGCGGGGCGCGACGGGCGAAACGAGAACATTTACG |
| | GGGAACAGCAAGTCTCCGAAGTCCCCTGTCAAAGGAGAATGTTGA (SEQ |
| | ID NO: 365) |
| DddA | >ACR30728.1 Rhs family protein [Burkholderia |
| homolog in | glumae BGR1] |
| Burkholderia | MYEAARVTDPIEHTSALTGFLVGAVLGIALIAAVAFATFTCGFGVALLAGMA |
| glumae | AGIGAQVLLSLGESIGKMFSSQSGAITLGSPNVYVNGKPTAYAMLSSVTCSK |
| BGR1 | HNPTPLVAQGSTNIFINGKPAARKDDKITCGATISDGSHDTYFHGGTQTCLPI |
| PROTEIN | DDEVPPWLRTATDWAFALAGLVGGLGGLLKEAGGLSRAVMPCAAKFIGGY |
| | VLGEAASRYVVGPAINSAIGGMFGNPVDVTTGRKILLAESETDYVVPSPMPV |
| | AIRRFYSSDLDYVGTLGRGWVLPWELRLHARDGRLWYTDAQGRESGFPML |
| | QPGHAAFSEADQRYLTCTPDGRYILHDLGETYYDFGHYEPGSGRIGWVRRIE |
| | DQAGQWCQFERDSRGRVREIQTCGGLLAVLDYEPEHGRLAGVSLVSGDQR |
| | RLVVAYGYDEHGQMASVTDANGALVRRFTYADGRMTSHSNALGFTSGYT |
| | WQAVGGAPRVVATHTSEGEAWAFEYDIEGRRTHVRHADGRHAQWRYDAQ |
| | FQIVEYLDFDGRRYGLKYNDAGMPVMLTLPGERTVTFEYDDAGRIVAETDP |
| | LGRTTKTRYDGNSRRPVEIIAPDGSAWHAEYDRQGRLLATRDPLDRENRYE |
| | YPKALSALPIAHVDALGGRKTFEWNRLGELVAYTDCSGKTTRNFYDAFGLP |
| | LARENALGHRVTFDLRPTGEARRVTYPDGSTESYEYDAAGLMIRHVGLGGR |
| | TQIALRNARGQIVEAVDPAGRRTCYRYDAEGRLRELQQGHARYAFTYSAGG |
| | RLTSETRPDGVRRRFEYGEAGDLAALDIVGAADDATANDRPVRTIRFERDR |
| | MGNLCAQHTPTEVTRYTRDTGGRLLEVACVPTAAGLALGIAPDTLTFEYDK |
| | AGRLSAEHGANGSVRYTLDALDNVMKLALPHEQTLQMLRYGSGHVHQIRC |
| | GDQVVSDFERDDLHRELTRTQGRLTERTAYDLLGRKIWQSAGFQPDALARG |
| | QGQVWRNYGYDAAGELAESHDSLRGSTQFSYDPAGYLTQRVNTADRQLES |
| | FAWDAAGNLLDDAQRRSRGYVEGNRLRMWQNLRFEYDPFGNLATKLRGA |
| | NQRQQFTYDGQDRLVAVRTQDARGVVETRFAYDPLGRRIAKTDIVRDARG |
| | VALREETKRFVWEGLRLAQEVRDTGVSSYVYSPDAPYTPAARVDAVLAEA |
| | MAAAAIEQARQATRIYHFHTDVSGAPQEATNEAGDIVWAGQYSAWGKVAP |
| | NQHAPARIDOPLRYAGQYADDSTELHYNTFRFYDPDVGRFINQDPIGLMGG |
| | LNLYQYAPNSIAWTDWWGLAGSYTLGSYQISAPQLPAYNGQTVGTFYYVN |
| | GAGGLESRTFSSGGPTPYPNYANAGHVEGQSALFMRDNGISDGLVFHNNPE |
| | GTCGFCVNMTETLLPENSKLTVVPPEGAIPVKRGATGETRTFTGNSKSPKSPV |
| | KGEC (SEQ ID NO: 364) |
| DddA | >bglu_2g02600 NC_012721.2:303868-308145 |
| homolog in | Burkholderia glumae BGR1 chromosome 2, complete |
| Burkholderia | sequence |
| glumae | GTGTACGAAGCGGCCCGCGTCACCGACCCGATCGAACACACCAGCGCGC |
| BGR1 | TGACCGGCTTTCTGGTGGGCGCCGTGCTCGGCATTGCCCTGATCGCCGCG |
| DNA | GTGGCGTTCGCCACCTTCACCTGCGGCTTCGGCGTGGCGCTGCTGGCCGG |
| | CATGGCCGCCGGCATCGGCGCGCAGGTGCTGTTGTCGTTAGGAGAATCG |
| | ATCGGGAAGATGTTCAGTTCGCAATCCGGCGCGATCACGCTCGGCTCGCC |
| | GAACGTCTATGTGAACGGCAAGCCGACCGCCTACGCCATGCTCAGCAGC |
| | GTGACGTGCAGCAAGCACAACCCGACGCCGCTCGTCGCGCAGGGGTCCA |
| | CCAACATCTTCATCAACGGCAAGCCGGCCGCCCGCAAGGACGACAAGAT |
| | CACCTGCGGCGCGACCATCTCCGACGGCTCGCACGACACCTATTTCCACG |
| | GCGGCACCCAGACCTGCCTGCCGATCGACGACGAAGTGCCGCCGTGGCT |
| | GCGCACCGCCACCGACTGGGCGTTCGCGCTGGCCGGGCTGGTGGGCGGG |
| | CTCGGCGGCCTGCTCAAGGAAGCGGGCGGGCTGTCGCGCGCGGTGATGC |
| | CGTGCGCGGCGAAGTTCATCGGCGGCTACGTGCTCGGCGAGGCGGCGAG |
| | CCGCTACGTGGTCGGCCCGGCCATCAACAGCGCGATCGGCGGGATGTTC |
| | GGCAACCCGGTGGACGTCACCACCGGGCGCAAGATCCTGCTGGCGGAAT |
| | CGGAAACCGATTACGTGGTGCCCAGCCCGATGCCGGTGGCGATCCGGCG |
| | CTTCTATTCGAGCGACCTCGACTACGTCGGCACGCTCGGGCGCGGCTGGG |
| | TGCTGCCGTGGGAACTGCGGCTGCACGCGCGCGACGGGCGGCTCTGGTA |
| | CACCGACGCGCAGGGGCGCGAGAGCGGCTTCCCGATGCTCCAGCCGGGC |
| | CATGCCGCGTTCAGCGAGGCCGACCAGCGCTATCTGACCTGCACCCCGG |
| | ATGGCCGCTACATCCTGCACGACCTCGGCGAAACCTATTACGACTTCGGC |
| | CACTACGAGCCGGGCTCGGGCCGCATCGGCTGGGTGCGCCGCATCGAGG |
| | ATCAGGCCGGCCAGTGGTGCCAGTTCGAGCGCGACAGCCGCGGCCGCGT |
| | GCGCGAAATCCAGACCTGCGGCGGCTTGCTGGCCGTGCTCGATTACGAG |
| | CCGGAACACGGGCGGCTCGCCGGGGTGTCGCTCGTCAGCGGGGATCAGC |
| | GCCGCCTCGTGGTGGCTTACGGCTATGACGAGCACGGCCAGATGGCGTC |
| | CGTGACCGATGCGAACGGCGCGCTGGTGCGCCGCTTCACCTATGCCGAC |
| | GGGCGCATGACGAGCCATTCGAACGCGCTCGGCTTCACGTCGGGCTATA |
| | CGTGGCAAGCCGTCGGCGGCGCGCCGCGGGTGGTTGCCACCCACACCAG |
| | CGAGGGCGAGGCCTGGGCCTTCGAGTACGACATTGAAGGACGCCGCACC |
| | CACGTGCGTCACGCCGACGGCCGCCACGCGCAATGGCGCTACGACGCGC |
| | AATTCCAGATCGTCGAGTACCTCGATTTCGACGGCCGGCGCTACGGGCTC |
| | AAGTACAACGACGCCGGCATGCCCGTGATGCTGACGCTGCCCGGCGAAC |
| | GGACCGTGACGTTCGAGTACGACGATGCCGGCCGCATCGTCGCCGAAAC |
| | CGATCCACTCGGCCGCACCACGAAAACGCGCTACGACGGCAACAGCAGG |
| | CGGCCCGTCGAGATCATCGCGCCCGACGGCAGCGCCTGGCACGCCGAAT |
| | ACGACCGGCAAGGCCGGCTGCTCGCCACCCGCGATCCGCTCGACCGGGA |
| | AAACCGCTACGAATACCCGAAGGCGCTCAGCGCGCTGCCGATCGCGCAC |
| | GTCGATGCGCTGGGCGGGCGCAAGACGTTCGAGTGGAACCGGCTCGGCG |
| | AGCTGGTGGCCTATACCGATTGCTCGGGCAAGACCACACGCAATTTTTAC |
| | GACGCATTCGGTCTGCCGCTCGCGCGCGAGAACGCGCTCGGCCACCGCG |
| | TGACGTTCGACCTGCGCCCGACCGGCGAGGCGCGGCGCGTCACCTATCCC |
| | GACGGCAGTACAGAAAGCTACGAATACGACGCCGCCGGGCTGATGATCC |
| | GGCACGTCGGGCTGGGCGGCCGGACGCAGATTGCGCTGCGCAACGCGCG |
| | TGGGCAGATCGTGGAGGCGGTCGATCCGGCCGGACGGCGCACCTGCTAC |
| | CGCTACGACGCCGAGGGGCGGCTGCGCGAGCTGCAACAGGGGCACGCGC |
| | GTTACGCGTTCACCTACAGCGCGGGCGGGCGGCTCACCAGCGAAACCCG |
| | GCCCGACGGCGTGCGGCGCCGCTTCGAATACGGCGAGGCCGGCGATCTG |
| | GCGGCGCTCGACATCGTCGGCGCGGCCGACGACGCCACGGCGAACGATC |
| | GTCCGGTTCGCACCATCCGCTTCGAGCGCGACCGCATGGGCAATCTGTGC |
| | GCGCAGCACACGCCCACCGAGGTGACGCGCTACACGCGCGACACCGGCG |
| | GCCGCCTGCTCGAAGTCGCATGCGTGCCGACCGCGGCCGGGCTGGCGCT |
| | CGGCATCGCGCCCGACACGCTGACCTTCGAATACGACAAGGCCGGGCGG |
| | CTGAGTGCCGAACACGGCGCGAACGGCAGCGTCCGATACACGCTCGACG |
| | CGCTCGACAACGTGATGAAGCTCGCCCTGCCGCACGAGCAGACGCTGCA |
| | GATGCTGCGCTACGGCTCGGGGCACGTGCATCAGATCCGCTGCGGCGAC |
| | CAGGTGGTCAGCGATTTCGAGCGCGACGACCTGCATCGCGAGCTGACGC |
| | GCACTCAGGGCCGCCTGACCGAGCGTACCGCCTACGACCTGCTGGGCCG |
| | CAAGATCTGGCAATCGGCCGGCTTCCAGCCCGACGCGCTTGCGCGCGGG |
| | CAGGGCCAGGTGTGGCGCAACTACGGCTACGACGCCGCCGGCGAACTGG |
| | CCGAGAGCCACGATAGCCTGCGCGGCAGCACGCAGTTCAGCTACGATCC |
| | GGCCGGCTATCTGACGCAGCGCGTCAATACCGCCGACCGGCAGCTCGAA |
| | TCGTTCGCCTGGGATGCCGCCGGCAACCTGCTCGACGATGCGCAGCGCCG |
| | CAGCCGCGGTTATGTCGAGGGCAACCGGCTGCGCATGTGGCAGAACCTG |
| | CGCTTCGAATACGACCCGTTCGGCAATCTCGCGACCAAGCTGCGCGGCGC |
| | GAACCAGCGCCAGCAGTTCACTTACGACGGGCAGGATCGGCTCGTGGCG |
| | GTGCGCACGCAGGACGCGCGCGGCGTGGTGGAGACGCGTTTCGCCTACG |
| | ATCCGCTGGGGCGGCGCATCGCCAAGACGGATATTGTGCGCGACGCGCG |
| | CGGCGTAGCGCTGCGCGAGGAAACGAAGCGGTTCGTGTGGGAGGGGCTG |
| | CGGCTCGCGCAGGAGGTGCGCGACACGGGCGTGAGCAGCTACGTGTACA |
| | GCCCGGACGCGCCCTATACGCCCGCGGCGCGCGTGGATGCCGTGCTGGC |
| | CGAGGCCATGGCCGCCGCTGCCATCGAGCAGGCCAGACAGGCGACGCGG |
| | ATCTATCACTTTCATACCGATGTGTCGGGCGCACCGCAAGAAGCGACGA |
| | ACGAGGCTGGCGACATTGTTTGGGCCGGCCAATACTCAGCCTGGGGCAA |
| | GGTGGCGCCGAACCAGCATGCCCCCGCCCGGATCGATCAGCCGCTCCGC |
| | TACGCCGGACAATATGCCGACGACAGTACCGAGCTGCACTACAACACGT |
| | TTCGTTTCTACGATCCGGACGTCGGCCGGTTTATCAATCAGGATCCAATC |
| | GGGTTGATGGGGGGGCTGAATCTTTACCAATATGCACCCAACTCGATCGC |
| | ATGGACCGACTGGTGGGGGCTGGCCGGCAGCTATACGCTCGGTTCCTATC |
| | AAATTTCTGCGCCTCAACTTCCGGCCTACAATGGACAGACTGTTGGGACC |
| | TTCTACTACGTGAACGGCGCGGGCGGGCTCGAATCGAGGACATTCTCTTC |
| | CGGAGGGCCGACCCCTTATCCAAATTATGCCAATGCCGGGCACGTGGAG |
| | GGCCAGTCCGCGCTGTTCATGAGGGATAACGGAATTTCAGACGGACTGG |
| | TTTTCCACAACAACCCTGAGGGCACTTGCGGATTCTGCGTTAATATGACC |
| | GAAACGCTTTTGCCTGAAAATTCCAAACTTACCGTCGTTCCGCCCGAGGG |
| | CGCGATCCCGGTCAAGCGGGGCGCGACGGGCGAAACGAGAACATTTACG |
| | GGGAACAGCAAGTCTCCGAAGTCCCCTGTCAAAGGAGAATGTTGA (SEQ |
| | ID NO: 366) |
| DddA | >AOT60363.1 tRNA nuclease WapA precursor |
| homolog in | [Streptomyces rubrolavendulae] |
| Streptomyces | MSSSDAGRAFGVPENVLARFTRYPGGARRRAGRTARARRLGIVLSAVLSAT |
| rubrolavendulae | LLPAEAWAIAPPAPRTGPTLDALQQEEEVDPDPAAMEELDDWDGGPVEPPA |
| PROTEIN | DYTPTEVTPPTGGTAPVPLDSAGEELVPAGTLPVRIGQASPTEEDPAPPAPSG |
| | TWDVTVEPRATTEAAAVDGAIIKLTPPASGSTPVDVELDYGRFEDLFGTEWS |
| | SRLKLTQLPECFLTTPELEECGTPITIPTSNDPATGTVRATVDPADGQPQGLA |
| | AQSGGGPAVLAATDSASGAGGTYKATSLSATGSWTAGGSGGGFSWSYPLTI |
| | PDTPAGPAPKISLSYSSQSVDGRTSVANGQASWIGDGWDYHPGFVERRYRSC |
| | NDDRSGTPNNDNSADKEKSDLCWASDNVVMSLGGSTTELVRDDTTGTWVA |
| | QNDTGARIEYKDKDGGALAAQTAGYDGEHWVVTTRDGTRYWFGRNTLPG |
| | RGAPTNSALTVPVFGNHTGEPCHAATYAASSCTQAWRWNLDYVEDVHGNA |
| | MVVDWKKEQNRYAKNEKFKAAVSYDRDAYPTQILYGLRADDLAGPPAGK |
| | VVFHAAPRCLESAATCSEAKFESKNYADKQPWWDTPATLHCKAGDENCYV |
| | TSPTFWSRVRLSAIETQGQRTPGSTALSTVDRWTLHQSFPKQRTDTHPPLWL |
| | ESITRVGFGRPDASGNQSSKALPAVTFLPNKVDMPNRVLKSTTDQTPDFDRL |
| | RVEVIRTETGGETHVTYSAPCPVGGTRPTPASNGTRCFPVHWSPDPAAFSDE |
| | NLDKSGYEPPLEWFNKYVVTKVTEMDLVAEQPSVETVYTYEGDAAWAKNT |
| | DEYGKPALRTYDQWRGYASVVTRTGTTANTGAADATEQSQTRTRYFRGMS |
| | GDAGRAKVHVTLTDVTGTATTVEDLLPYQGMAAETLTYTKAGGDVAAREL |
| | AFPYSRKTASRARPGLPALEAYRTGTTRTDSIQHISGDRTRAAQNHTTYDDA |
| | YGLPTQTYSLTLSPNDSGTLVAGDERCTVTTYVHNTAAHIIGLPDRVRATTG |
| | DCAAAPNATTGQIVSDSRTAYDALGAFGTAPVKGLPVQVDTISGGGTSWITS |
| | ARTEYDALGRATKVTDAAGNSTTTTYSPATGPAFEVTVTNAAGHATTTTLD |
| | PGRGSALTVTDQNGRKTTSTYDELGRATGVWTPSRPVNQDASVRFVYQIED |
| | SKVPAVHTRVLRDAGTYEESIELYDGFLRPRQTQREALGGGRIVTETLYNAN |
| | GSAKEVRDGYLAEGEPARELFVPLSLDQVPSATRTAYDGLGRPVRTTTLHR |
| | GVPRHSATTAYGGDWELSRTGMSPDGTTPLSGSRAVKATTDALGRPARIQH |
| | FTTQNVSAESVDTTYTYDPRGPLAQVTDAQQNTWTYTYDARGRKTSSTDPD |
| | AGAAYFGYNALDQQVWSKDNQGRLQYTTYDVLGRQTELRDDSASGPLVA |
| | KWTFDTLPGAKGHPVASTRYNDGAAFTSEVTGYDTEYRPTGNKVTIPSTPM |
| | TTGLAGTYTYASTYTPTGKVQSVDLPATPGGLAAEKVITRYDGEDSPTTMSG |
| | LAWYTADTFLGPYGEVLRTASGEAPRRVWTTNVYDEDTRRLTRTTAHRET |
| | APHPVSTTTYGYDTVGNITSIADQQPAGTEEQCFSYDPMGRLVHAWTDGNS |
| | AVCPRTSTAPGAGPARADVSAGVDGGGYWHSYAFDAIGNRTKLTVHDRTD |
| | AALDDTYTYTYGKTLPGNPQPVQPHTLTQVDAVLNEPGSRVEPRSTYAYDT |
| | SGNTTQRVIGGDTQTLAWDRRNKLTSVDTNNDGTPDVKYLYDASGNRLVE |
| | DDGTTRTLFLGEAEIVVNTAGQAVDARRYYSSPGAPTTIRTTGGKTTGHKLT |
| | VMLSDHHSTATTAVELTDTQPVTRRRFDPYGNPRGTEPTTWPDRRTYLGVG |
| | IDDPATGLTHIGAREYDASTGRFISVDPVMDLTDPLQMNGYTYANADPINNS |
| | DPTGLLLDARGGGTQKCVGTCVKDVTNRKGIPLPPGEEWKHEGEAQTDFNG |
| | DGFITVFPTVNVPAKWKKAKKYTEAFYKAVDTACFYGRESCADPEYPSRAH |
| | SINNWKGKACKAVGGKCPERLSWGEGPAFAGGFAIAAEEYAGRGGYRGGG |
| | ARRGSPCKCFLAGTEVLMADGSTKSIEDIKLGDEVVATDPVTGEAGAHPVSA |
| | LIATENDKRFNELVIITSEGVERLTATHEHPFWSPSEGEWLEAGELRTGMTLR |
| | SDSGETLVVAGNRAFTQRARTYNLTVADLHTYYVLAGQTPVLVHNANCGP |
| | HLKDLQKDYPRRTVGILDVGTDQLPMISGPGGQSGLLKNLPGRTKANGEHV |
| | ETHAAAFLRMNPGVRKAVLYIDYPTGTCGTCRSTLPDMLPEGVQLWVISPR |
| | RTEKFTGLPD (SEQ ID NO: 367) |
| DddA | >A4G23_03234 CP017316.1:3756245-3763321 |
| homolog in | Streptomyces rubrolavendulae strain MJM4426, |
| Streptomyces | complete genome |
| rubrolavendulae | ATGTCCTCGTCCGATGCGGGACGCGCCTTCGGCGTGCCCGAAAACGTCCT |
| DNA | GGCGCGTTTCACGCGGTATCCCGGCGGGGCGCGACGCCGTGCCGGGCGC |
| | ACGGCGCGCGCCCGGCGCCTGGGCATCGTGCTGTCCGCCGTCCTCTCGGC |
| | GACCCTGCTGCCCGCCGAGGCATGGGCCATCGCGCCCCCGGCGCCGCGC |
| | ACCGGTCCGACCCTGGACGCCCTCCAGCAGGAGGAGGAGGTCGATCCGG |
| | ACCCGGCCGCCATGGAAGAGCTGGACGACTGGGACGGTGGGCCGGTCGA |
| | GCCCCCGGCCGACTACACCCCCACCGAGGTCACGCCTCCCACCGGCGGC |
| | ACCGCCCCGGTGCCGCTGGACAGCGCGGGCGAGGAACTGGTCCCGGCCG |
| | GGACCCTGCCCGTGCGCATCGGCCAGGCGTCCCCCACCGAGGAGGACCC |
| | GGCACCCCCGGCACCCAGCGGCACGTGGGACGTCACCGTGGAGCCCCGC |
| | GCCACCACCGAGGCGGCCGCCGTGGACGGCGCCATCATCAAGCTCACCC |
| | CGCCCGCCAGCGGCTCCACACCGGTCGACGTGGAACTCGACTACGGCCG |
| | GTTCGAGGACCTGTTCGGCACCGAGTGGTCCTCCCGGCTCAAGCTGACGC |
| | AGCTCCCGGAGTGCTTCCTCACGACGCCCGAGCTGGAGGAGTGCGGCAC |
| | CCCCATCACCATCCCGACGAGCAACGACCCGGCCACCGGGACGGTCCGG |
| | GCCACCGTCGACCCGGCCGACGGGCAGCCGCAGGGCCTGGCCGCGCAGT |
| | CGGGCGGCGGTCCCGCCGTCCTCGCCGCGACCGACTCGGCGTCCGGCGC |
| | CGGCGGCACGTACAAGGCGACCTCCCTCTCGGCCACCGGCTCCTGGACG |
| | GCCGGCGGCAGCGGCGGCGGCTTCTCCTGGTCGTATCCGCTCACCATCCC |
| | GGACACCCCGGCCGGCCCCGCGCCGAAGATCTCCCTGTCGTACTCCTCCC |
| | AGTCCGTCGACGGCCGCACCTCCGTCGCCAACGGCCAGGCGTCGTGGAT |
| | AGGCGACGGCTGGGACTACCACCCCGGCTTCGTCGAGCGCCGCTACCGC |
| | TCCTGCAACGACGACCGCTCCGGCACCCCGAACAACGACAACAGTGCGG |
| | ACAAGGAGAAGTCCGACCTGTGCTGGGCGAGCGACAACGTCGTGATGTC |
| | GCTCGGCGGCTCCACCACCGAACTCGTCCGCGACGACACGACCGGCACG |
| | TGGGTCGCGCAGAACGACACCGGTGCCCGGATCGAGTACAAGGACAAGG |
| | ACGGCGGAGCCCTGGCCGCCCAGACCGCCGGCTACGACGGCGAGCACTG |
| | GGTCGTCACCACCCGCGACGGAACCCGCTACTGGTTCGGCCGCAACACC |
| | CTCCCCGGCCGCGGCGCCCCCACGAACTCCGCCCTCACCGTCCCCGTCTT |
| | CGGCAACCACACCGGCGAGCCCTGCCACGCCGCCACCTACGCCGCCTCCT |
| | CCTGCACCCAGGCGTGGCGCTGGAACCTCGACTACGTCGAGGACGTCCA |
| | CGGCAACGCGATGGTCGTCGACTGGAAGAAGGAGCAGAACCGGTACGCG |
| | AAGAACGAGAAGTTCAAGGCGGCTGTCTCCTACGACCGCGACGCGTATC |
| | CGACGCAGATCCTCTACGGCCTGCGCGCCGACGACCTGGCGGGCCCGCC |
| | CGCCGGCAAGGTCGTCTTCCACGCCGCCCCGCGCTGCCTCGAAAGCGCG |
| | GCCACCTGCTCCGAAGCCAAGTTCGAGTCCAAGAACTACGCGGACAAGC |
| | AGCCCTGGTGGGACACACCGGCCACCCTGCACTGCAAGGCCGGTGACGA |
| | GAACTGCTACGTCACCTCGCCGACGTTCTGGAGCCGCGTCCGCCTGTCGG |
| | CGATCGAGACGCAGGGTCAGCGCACGCCCGGCTCGACGGCGCTGTCCAC |
| | GGTCGACCGCTGGACCCTGCACCAGTCGTTCCCGAAGCAGCGCACCGAC |
| | ACCCACCCGCCGCTCTGGCTGGAGTCGATCACCCGCGTGGGCTTCGGCCG |
| | GCCGGACGCCTCCGGCAACCAGTCGAGCAAGGCCCTCCCGGCGGTGACC |
| | TTCCTGCCCAACAAGGTCGACATGCCGAACCGCGTGCTGAAGAGCACGA |
| | CGGACCAGACGCCCGATTTCGACCGCCTGCGCGTCGAGGTCATCCGCAC |
| | GGAGACCGGCGGCGAGACCCATGTGACGTACTCCGCCCCCTGCCCCGTC |
| | GGCGGCACCCGCCCCACCCCGGCCTCCAACGGCACCCGCTGCTTCCCGGT |
| | CCACTGGTCCCCCGACCCGGCGGCCTTCTCCGACGAGAACCTGGACAAG |
| | AGCGGCTACGAGCCGCCCCTCGAGTGGTTCAACAAGTACGTCGTCACCA |
| | AGGTCACCGAGATGGACCTCGTGGCGGAGCAGCCCAGCGTCGAGACCGT |
| | CTACACCTACGAGGGCGACGCCGCCTGGGCGAAGAACACCGACGAGTAC |
| | GGCAAGCCCGCCCTGCGCACCTACGACCAGTGGCGCGGCTACGCGAGCG |
| | TCGTCACCCGCACGGGCACCACGGCCAACACCGGCGCCGCCGACGCCAC |
| | CGAGCAGTCCCAGACCCGCACCCGGTACTTCCGCGGCATGTCCGGCGAC |
| | GCGGGCCGCGCCAAGGTGCACGTCACGCTCACGGACGTGACCGGCACCG |
| | CGACCACCGTCGAGGACCTGCTCCCGTACCAGGGCATGGCCGCCGAGAC |
| | CCTTACCTACACCAAGGCGGGCGGCGACGTCGCCGCCCGCGAGCTGGCC |
| | TTCCCCTACAGCAGGAAGACCGCCTCCCGCGCCCGCCCCGGCCTCCCCGC |
| | CCTGGAGGCGTACCGCACGGGCACGACGCGCACGGACTCCATCCAGCAC |
| | ATCAGCGGCGACCGGACGCGCGCCGCTCAGAACCACACCACATACGACG |
| | ACGCGTACGGCCTGCCCACCCAGACCTACTCGCTGACACTCTCGCCGAAC |
| | GACTCCGGCACCCTTGTCGCCGGTGACGAGCGGTGCACCGTCACGACGT |
| | ACGTCCACAACACCGCCGCGCACATCATCGGCCTCCCCGACCGCGTCCGC |
| | GCCACGACGGGCGACTGCGCCGCCGCGCCGAACGCCACCACCGGCCAGA |
| | TCGTCTCCGACAGCCGCACCGCGTACGACGCGCTCGGCGCCTTCGGCACG |
| | GCCCCGGTCAAGGGCCTGCCGGTCCAGGTGGACACGATCTCCGGAGGCG |
| | GCACGAGCTGGATCACCTCGGCGCGCACGGAGTACGACGCGCTGGGCCG |
| | TGCGACCAAGGTCACCGACGCGGCGGGCAACTCCACCACGACCACGTAC |
| | AGCCCGGCGACCGGCCCCGCGTTCGAGGTCACCGTGACCAACGCGGCTG |
| | GTCATGCCACGACCACCACCCTCGACCCCGGTCGCGGCTCGGCGCTGACC |
| | GTCACCGACCAGAACGGCCGCAAGACCACCAGCACGTACGACGAACTCG |
| | GCCGGGCCACCGGCGTGTGGACGCCCTCCCGCCCGGTGAACCAGGACGC |
| | GTCCGTGCGCTTCGTCTACCAGATCGAGGACAGCAAGGTCCCGGCGGTG |
| | CACACTCGGGTCCTGCGCGACGCCGGTACGTACGAGGAGTCGATCGAGC |
| | TCTACGACGGCTTCCTCCGCCCCCGTCAGACCCAGCGCGAGGCGCTGGGC |
| | GGCGGCCGAATCGTCACCGAGACCCTCTACAACGCCAACGGCTCTGCGA |
| | AGGAAGTGCGCGACGGCTACCTGGCGGAGGGCGAGCCCGCGCGGGAACT |
| | GTTCGTCCCGCTCTCCCTCGACCAGGTGCCGAGCGCGACGAGGACGGCCT |
| | ATGACGGCCTGGGCCGGCCCGTCCGGACGACGACCCTCCACAGGGGAGT |
| | CCCCCGGCACTCCGCCACCACGGCGTACGGCGGCGACTGGGAACTGAGC |
| | CGCACCGGCATGTCGCCCGACGGAACGACGCCGCTCTCTGGCAGCCGCG |
| | CCGTGAAGGCGACGACGGACGCGCTCGGCCGCCCGGCCCGCATCCAGCA |
| | CTTCACCACCCAGAACGTGTCGGCCGAGAGCGTCGACACCACGTACACC |
| | TACGACCCCCGCGGCCCCCTTGCCCAGGTCACCGACGCCCAGCAGAACA |
| | CCTGGACGTACACGTACGACGCCCGTGGGCGCAAGACGTCCTCCACCGA |
| | CCCGGACGCGGGCGCCGCCTACTTCGGCTACAACGCGCTGGACCAGCAG |
| | GTCTGGTCGAAGGACAACCAGGGCCGCCTGCAGTACACGACGTACGACG |
| | TCCTGGGCCGCCAGACCGAGCTGCGCGACGACTCCGCGTCCGGCCCGCT |
| | GGTGGCGAAGTGGACCTTCGACACCCTGCCGGGCGCCAAGGGCCACCCG |
| | GTCGCGTCGACCCGCTACAACGACGGCGCCGCGTTCACCAGCGAGGTGA |
| | CCGGTTACGACACCGAGTACCGTCCGACCGGCAACAAGGTCACCATCCC |
| | CAGCACCCCGATGACCACGGGCCTCGCCGGCACGTACACGTACGCCAGC |
| | ACGTACACCCCGACCGGCAAGGTCCAGTCCGTCGACCTGCCCGCGACGC |
| | CCGGCGGGCTCGCCGCGGAGAAGGTGATCACCCGCTACGACGGCGAGGA |
| | CTCGCCCACCACGATGTCGGGCCTGGCCTGGTACACGGCCGACACCTTCC |
| | TCGGCCCGTACGGGGAAGTGCTGCGCACGGCGTCGGGCGAGGCCCCGCG |
| | CCGCGTGTGGACGACCAACGTCTACGACGAGGACACCCGCCGCCTCACC |
| | AGGACCACCGCGCACCGGGAGACGGCTCCCCACCCGGTCAGCACGACCA |
| | CCTACGGCTACGACACGGTCGGCAACATCACGTCCATCGCCGACCAGCA |
| | GCCGGCGGGTACCGAGGAGCAGTGCTTCTCGTACGACCCGATGGGGCGC |
| | CTCGTCCACGCCTGGACGGACGGCAACAGCGCCGTCTGCCCCAGGACGT |
| | CCACGGCACCGGGCGCCGGCCCGGCCCGCGCCGACGTCTCGGCCGGTGT |
| | CGACGGCGGCGGATACTGGCACTCGTACGCGTTCGACGCGATCGGCAAC |
| | CGGACGAAGCTGACCGTCCACGACCGCACCGACGCGGCCCTGGACGACA |
| | CGTACACCTACACCTACGGCAAGACCCTGCCGGGTAACCCGCAGCCGGT |
| | CCAGCCGCACACCCTCACCCAGGTCGACGCGGTGCTCAACGAGCCCGGA |
| | TCGAGAGTCGAACCGCGCTCCACATACGCCTACGACACCTCCGGCAACA |
| | CCACCCAGCGCGTCATCGGCGGCGACACCCAGACCCTGGCCTGGGACCG |
| | CCGCAACAAGCTGACGTCCGTCGACACGAACAACGACGGCACACCGGAC |
| | GTGAAGTACCTGTACGACGCGTCGGGCAACCGCCTGGTCGAGGACGACG |
| | GCACCACGCGCACCCTCTTCCTCGGCGAGGCCGAGATCGTCGTCAACACG |
| | GCCGGCCAGGCCGTGGACGCGCGCCGCTACTACAGCAGCCCCGGCGCCC |
| | CGACGACGATCCGCACGACCGGCGGCAAGACCACGGGCCACAAGCTGAC |
| | CGTCATGCTGTCGGACCACCACAGCACGGCGACGACCGCGGTCGAGCTG |
| | ACCGACACCCAGCCGGTCACCCGCCGCCGCTTCGACCCGTACGGCAACC |
| | CCCGCGGCACCGAGCCGACCACCTGGCCCGACCGCCGCACCTACCTGGG |
| | CGTCGGCATCGACGACCCCGCCACGGGCCTGACCCACATCGGCGCCCGC |
| | GAATACGACGCATCGACGGGCCGCTTCATCTCCGTCGATCCGGTCATGGA |
| | CCTCACGGACCCGCTCCAGATGAACGGGTACACCTACGCCAACGCGGAC |
| | CCGATCAACAACAGCGACCCCACCGGACTGTTGCTCGACGCCCGAGGCG |
| | GCGGCACTCAGAAGTGCGTGGGAACCTGCGTCAAGGACGTCACGAACCG |
| | AAAGGGAATTCCGCTCCCGCCTGGCGAGGAGTGGAAGCATGAAGGGGAG |
| | GCGCAAACCGATTTCAACGGTGACGGCTTCATCACCGTCTTCCCGACCGT |
| | GAATGTTCCGGCGAAGTGGAAGAAGGCGAAGAAGTACACGGAGGCTTTC |
| | TACAAGGCGGTTGATACTGCTTGCTTCTATGGACGCGAAAGCTGTGCGGA |
| | TCCGGAGTACCCTTCGCGGGCGCATAGCATCAACAACTGGAAGGGAAAG |
| | GCATGCAAAGCCGTAGGGGGAAAATGCCCTGAGAGGTTGTCGTGGGGGG |
| | AGGGTCCGGCGTTCGCTGGTGGCTTCGCGATAGCAGCGGAAGAGTATGC |
| | GGGGAGAGGGGGCTACCGGGGCGGTGGGGCGAGGAGGGGGTCGCCCTG |
| | TAAGTGCTTCCTTGCCGGCACCGAGGTGCTCATGGCGGATGGCAGCACTA |
| | AAAGTATCGAGGACATCAAGCTCGGTGACGAAGTGGTTGCGACTGATCC |
| | GGTAACCGGTGAGGCCGGTGCGCACCCTGTCTCGGCGCTGATCGCCACC |
| | GAGAACGACAAGCGTTTCAACGAGCTGGTCATTATCACCAGCGAGGGTG |
| | TAGAGCGTCTTACCGCAACGCATGAGCACCCCTTCTGGTCGCCATCCGAA |
| | GGGGAGTGGTTGGAGGCGGGTGAGCTGCGCACTGGCATGACGCTGCGCT |
| | CCGACTCTGGCGAAACTCTCGTAGTCGCAGGAAACCGCGCCTTCACCCAG |
| | CGAGCCCGGACCTACAACCTCACGGTTGCAGACCTCCACACGTACTATGT |
| | GCTGGCGGGCCAGACTCCGGTACTGGTTCACAATGCAAACTGTGGACCTC |
| | ACCTGAAGGACCTGCAAAAGGACTACCCCCGGCGCACTGTGGGCATCCT |
| | TGACGTCGGAACTGATCAGCTCCCGATGATTAGCGGCCCAGGTGGCCAG |
| | TCGGGACTTCTCAAGAACCTCCCAGGTCGTACGAAGGCCAACGGGGAGC |
| | ACGTGGAGACTCACGCAGCAGCGTTCTTGCGTATGAACCCGGGTGTCAG |
| | AAAGGCCGTGCTCTACATCGACTACCCGACGGGGACCTGCGGAACATGT |
| | AGAAGTACATTGCCTGACATGCTGCCCGAGGGTGTTCAGTTGTGGGTGAT |
| | CTCGCCGCGTAGGACTGAAAAATTCACGGGACTTCCTGACTGA (SEQ ID |
| | NO: 368) |
| DddA | >AVT32940.1 hypothetical protein C6361_29650 |
| homolog in | [Plantactinospora sp. BC1] |
| Plantactinospora | MGDRLPAFVDGGDTLGIFSRGGIERDLASGVAGPASSLPKGTPGFNGLVKSH |
| sp. BC1 | VEGHAAALMRQNGIPNAELYINRVPCGSGNGCAAMLPHMLPEGATLRVYG |
| PROTEIN | PNGYDRTFTGLPD (SEQ ID NO: 369) |
| DddA | >C6361_29650 CP028158.1:6764267-6764614 |
| homolog in | Plantactinospora sp. BC1 chromosome, complete |
| Plantactinospora | genome |
| sp. BC1 | CTGGGTGACCGGCTCCCTGCCTTCGTGGACGGTGGAGACACGTTGGGCAT |
| DNA | CTTTTCTCGCGGAGGTATTGAGCGGGACCTCGCCAGCGGAGTTGCGGGTC |
| | CTGCAAGTAGCCTTCCTAAAGGCACGCCTGGCTTCAATGGTCTTGTAAAG |
| | AGTCATGTTGAAGGGCATGCGGCTGCGCTAATGAGACAAAATGGAATTC |
| | CGAACGCTGAGCTGTATATCAACAGAGTGCCGTGCGGTTCAGGTAATGG |
| | CTGCGCAGCGATGTTGCCGCATATGCTTCCGGAAGGTGCCACCCTCCGCG |
| | TATATGGGCCGAACGGGTACGATAGAACCTTCACTGGACTTCCGGACTG |
| | A (SEQ ID NO: 370) |
| DddA | >BAJ27137.1 hypothetical protein KSE_13070 |
| homolog in | [Kitasatospora setae KM-6054] |
| Kitasatospora | MAAVPSAEALAAKRARDTIWTPPNTPLGSQTKSVDGENLVPGRLPGPLEPEP |
| setae KM- | ADWTPGGPASVPAPGSADVTLGFDSAEAAAARKATGGAAPASDGAALRAG |
| 6054 | SLPVVIGAAKDAKSGAHRIRVELVDQAKSRAAHLDSPLIALTDTEPDTPPSGR |
| PROTEIN | TTKVSLDLKGIGAQTWADRARLVALPACALETPDRPECQQQTPVQSSVDLR |
| | SGLLTAEVILPAATEGTAPPTKSSLGSGTASGVVQAGLTTAAPAKAAPTVLA |
| | ATAGASGSGGSFSATSLSPSAAWGAGSNVGNFTYSYPIQTPPSLGGTAPSVG |
| | LGYDSSAVDGKTSAQNSQSSWLGEGWGYEAGFIERGYKSCNTAGIANSSDM |
| | CWGGQNATLSLAGHSGTLVRDDTTGVWHLQSDDGTKIEQLTGAPNGLQNG |
| | EHWRITTTDGTQFYFGRNHLPGGDGTDPASNSAFKEPVYSPKSGDPCYNSST |
| | ATGSWCTMGWRWNLDYAVDVHGNLITYTYAQETNYYSRGAGQNSGSGTL |
| | TDYTRAGYLTQIAYGQRLSEQVTAKGAAKAAALITFTAAERCVPSGSITCTE |
| | AQRTTANASYWPDTPLDQVCASTGTCTRAGPTFFTTKRLASLTTQVLVSGA |
| | YRTVDTWTLTHSFKDPGDGNAKSLWLDSIQRTGTNGQTAVTMPPVTFTAV |
| | MKPNRVDGDLTLKDGTKVTVTPFNRPRLQQVTTETGGQINVVYTTSSDAAH |
| | PACSRLAGTMPAAADGNTLACAPVKWYLPGSSSPDPVDDWFNKYLISAVTE |
| | QDAISGTTLIKATNYTYNGDAAWHRNDAEFTDAKTRTWDGFRGYQSVTSTT |
| | GSAYPGEAPRTQQTATYLRGMDGDVKADGSTRSVQVANPLGGPALTDSPW |
| | LAGSSFATQTYDQAGGTVISANGSVAGGQQVTATHAQSGGMPALVARYPA |
| | SQVTTTSKSKLSDGTWRTNTTVSTSDPAHANRPLSSDDKGDGTPGAELCSTN |
| | GYATGTNPMMLNILAERTVTKGACGTPVTSANTVSSARTLYDGKPYGQAG |
| | DLAESTSALTLDHYDTGGNPVYVHTAASTFDAYGRLTSVSEANGATYDAAG |
| | NQLTAPNLTPATTRTAYTPATGAIATTVTQTTPTGWTTTLTQDPGRAEALVS |
| | TDANGRATTQQYDGLGRLTAAWSPERATNLTPSQKFSYAVNGTTGPSVVTS |
| | QWLKEAGGYAYKNELYDGLGRLRQVQRTSDTYSGRLITDTVYDSHGWPVK |
| | TASPYYEKTTAPNSTVYLPQDSQVPAQTWVTFDGIGRTTRSAFVSYGQQQW |
| | ATTTAYPGADRTDVTPPNGKYPTSTFTDGRNQVSALWQYRTATPTGNPADA |
| | TVTTYTYDAANRPATRKDAAGNTWSYGYDLRGRQTTVTDPDTGTTTTAYD |
| | VNSRAVSTTDGKGNTLVVSYDLIGRKTGLYQGSIAPANQLAGWTYDTLPGG |
| | KGKPTSSTRYVGGAGGSAYTQAVTGYDAGYRPTGTSVTIPASEGKLAGTYT |
| | TGLTYNPVLGTLKQTDLPAIGAAPAESVMYTYNISGVLQKSYSDTYYVYDV |
| | QYDAFGRPVRTTTGDAGTQVVSTQLDKTDYTYNQAGDVTSVTDVQNGTAT |
| | DAQCFTYDHLGRLTQAWTDTAGSTSTTSGTWTDTSGTVHNSGSSQSVPALG |
| | ACANANGPASTGSPAKLSVGGPSPYWQSYGYDSTGNRTTLVQHDTTGNTTK |
| | DTTTTQTFGPAGSVNTATGAPNTGGGTGGPHALLTSSTTGPTGTQVTSYQYD |
| | QLGNTTAVTETSGTTTLAWNGEDKLASVTKTGQAQATSYLYDADGNQLIRR |
| | NPGKTTLNLGSDEVTLDTAANSLTDTRYYSAPGGISIARTTGPTGASALAYQ |
| | ASDPHGTANVQINVDAAQTTTRRPTDPFGNPRGTQPAPNTWAGDKGFVGGT |
| | KDDTTGLTNLGAREYQPTTGRFLNPDPLLDAGNPQQWNGYAYSDNDPVNS |
| | SDPSGLITNALADGDTYVARPAAFCVTMSCVEQTSGPGFWEDKRVGDAVFA |
| | AVVQATTQSNGNGSSQTKKEKGIWGQAWDWTKKNGGAILGALVEGAVFST |
| | CFIGAGFAAPATGGITVIAGAAACGAVAGEAGALTTNILTPDADHSVDGITN |
| | DMVVGEITGAAVSAASEGASSLAKPAVRKLLGMEAEEGLEAAGRAATGPC |
| | NSFPAGVTVLLADGTTKPIEQIAQGDQVTATDPQTGTTQAEPVTDTIIGHDDT |
| | EFTDLTLTNDADPRAPPSEITSTTHHPYWNATTSRWTDAGDLKPGDHVRTPD |
| | GTELTVNTVYSYTTQPRTARNLTVADLHTYYVLAGNTPVLVHNTGPGCGEP |
| | GFVSDAANSLSGRRITTGQIFDASGNPIGPEITSGGGSLADRAQSYLADSPNIR |
| | NLPAKARYASADHVEAQYAVWMRENGVTDASVVINQNYVCGLPLGCQAA |
| | VPAILPRGSTMTVWYPGSGSPIVLRGVG (SEQ ID NO: 371) |
| DddA | >KSE_13070 NC_016109.1:1451556-1458878 |
| homolog in | Kitasatospora setae KM-6054 DNA, complete genome |
| Kitasatospora | GTGCTGGGGACAGCGGCCGCGCTCGCGGTCATGATGTCCATGGCGGCGG |
| setae KM- | TGCCGTCCGCCGAGGCACTGGCCGCGAAGCGGGCACGCGACACCATCTG |
| 6054 | GACGCCGCCCAACACCCCGCTGGGCAGCCAGACCAAGTCCGTCGACGGC |
| DNA | GAGAACCTCGTCCCGGGCCGCCTGCCCGGCCCCCTGGAGCCGGAACCGG |
| | CCGACTGGACACCCGGCGGACCGGCATCCGTGCCCGCTCCGGGCAGCGC |
| | GGACGTCACCCTCGGCTTCGACTCCGCGGAGGCCGCCGCCGCCCGCAAG |
| | GCCACCGGCGGCGCCGCCCCCGCCTCCGACGGCGCGGCCCTCCGCGCGG |
| | GCTCCCTCCCCGTCGTCATCGGCGCGGCGAAGGACGCCAAGAGCGGCGC |
| | CCACCGGATCCGCGTCGAGCTCGTGGACCAGGCCAAGAGCCGTGCCGCA |
| | CACCTCGACAGCCCGCTGATCGCACTCACCGACACCGAGCCGGACACCC |
| | CGCCCTCCGGTCGGACCACGAAGGTGTCCCTCGACCTGAAGGGCATCGG |
| | CGCCCAGACCTGGGCGGACCGCGCGCGACTCGTCGCCCTGCCCGCCTGC |
| | GCCCTGGAGACGCCCGACAGGCCCGAGTGCCAGCAGCAGACCCCCGTGC |
| | AGAGCTCCGTCGACCTGCGCTCCGGACTGCTGACGGCCGAGGTCATTCTG |
| | CCCGCCGCCACCGAGGGCACCGCCCCGCCCACCAAGAGCTCCCTCGGCT |
| | CGGGCACCGCCTCCGGCGTCGTCCAGGCCGGCCTCACCACGGCGGCGCC |
| | CGCCAAGGCCGCGCCCACGGTGCTCGCCGCGACCGCCGGCGCGTCCGGC |
| | TCGGGCGGCAGCTTCTCGGCGACCTCGCTGTCGCCCTCCGCGGCCTGGGG |
| | CGCCGGCTCCAACGTCGGCAACTTCACCTACTCGTACCCGATCCAGACGC |
| | CTCCCTCGCTCGGCGGGACCGCCCCCTCCGTGGGCCTCGGGTACGACTCG |
| | TCCGCCGTCGACGGGAAGACCTCCGCGCAGAACTCCCAGTCCTCCTGGCT |
| | CGGCGAGGGCTGGGGCTACGAGGCCGGGTTCATCGAGCGCGGCTACAAG |
| | TCCTGCAACACGGCCGGCATCGCGAACTCCTCGGACATGTGCTGGGGCG |
| | GGCAGAACGCCACCCTCTCGCTGGCCGGCCACTCCGGCACCCTGGTGCGC |
| | GACGACACCACCGGCGTCTGGCACCTGCAGAGCGACGACGGCACGAAGA |
| | TCGAACAGCTCACCGGCGCGCCCAACGGCCTGCAGAACGGCGAGCACTG |
| | GCGGATCACCACGACCGACGGCACGCAGTTCTACTTCGGCCGCAACCAC |
| | CTGCCCGGCGGCGACGGCACCGACCCGGCGAGCAACTCCGCCTTCAAGG |
| | AACCGGTGTACTCGCCCAAGAGCGGCGACCCCTGCTACAACTCCTCCACC |
| | GCCACCGGCTCCTGGTGCACGATGGGCTGGCGCTGGAACCTCGACTACG |
| | CCGTCGACGTCCACGGCAACCTGATCACCTACACCTACGCCCAGGAGAC |
| | CAACTACTACAGCCGAGGCGCCGGCCAGAACAGCGGCAGCGGCACCCTG |
| | ACCGACTACACCCGCGCCGGCTACCTCACCCAGATCGCCTACGGCCAGC |
| | GCCTGAGCGAGCAGGTCACCGCCAAGGGCGCGGCCAAGGCCGCTGCCCT |
| | CATCACCTTCACCGCCGCGGAACGCTGCGTCCCGTCCGGCTCGATCACCT |
| | GCACCGAGGCACAGCGCACGACCGCGAACGCCTCGTACTGGCCGGACAC |
| | CCCGCTCGACCAGGTCTGCGCCTCCACCGGCACCTGCACCCGGGCCGGCC |
| | CGACGTTCTTCACCACCAAGCGCCTCGCCTCCCTCACCACCCAGGTCCTG |
| | GTCTCCGGCGCCTACCGCACCGTCGACACCTGGACGCTCACCCATTCCTT |
| | CAAGGACCCGGGCGACGGCAACGCCAAGTCGCTGTGGCTCGACTCGATC |
| | CAGCGCACCGGCACCAACGGGCAGACCGCGGTCACCATGCCGCCCGTCA |
| | CCTTCACGGCGGTGATGAAGCCGAACCGGGTGGACGGGGACCTCACCCT |
| | CAAGGACGGCACCAAGGTCACCGTCACCCCGTTCAACCGGCCCCGCCTC |
| | CAGCAGGTCACCACGGAGACCGGCGGCCAGATCAACGTCGTCTACACCA |
| | CCTCCTCCGACGCCGCGCACCCCGCCTGCTCGCGCCTGGCCGGCACCATG |
| | CCCGCCGCGGCGGACGGCAACACCCTCGCCTGCGCCCCCGTCAAGTGGT |
| | ACCTGCCCGGATCCAGCTCCCCGGACCCGGTCGACGACTGGTTCAACAA |
| | GTACCTGATCAGCGCCGTCACCGAACAGGACGCGATCAGCGGCACCACC |
| | CTGATCAAGGCCACCAACTACACCTACAACGGCGACGCCGCCTGGCACC |
| | GCAACGACGCCGAGTTCACCGACGCCAAGACCCGCACCTGGGACGGCTT |
| | CCGCGGCTACCAGTCCGTCACCAGCACCACCGGCAGCGCCTACCCGGGC |
| | GAGGCCCCCAGGACCCAGCAGACCGCGACCTACCTGCGCGGCATGGACG |
| | GCGACGTCAAGGCCGACGGCTCCACCCGCAGCGTCCAGGTCGCCAACCC |
| | GCTCGGCGGCCCGGCCCTCACCGACAGCCCGTGGCTGGCCGGCTCCAGCT |
| | TCGCCACCCAGACCTACGACCAGGCCGGCGGCACCGTCATCTCCGCCAA |
| | CGGCTCCGTCGCCGGCGGCCAGCAGGTCACCGCCACCCACGCCCAGAGC |
| | GGCGGCATGCCGGCCCTGGTCGCCCGCTACCCCGCCTCCCAGGTCACCAC |
| | CACCTCCAAGTCCAAGCTCTCCGACGGGACCTGGCGCACCAACACCACC |
| | GTCAGCACCAGCGACCCCGCGCACGCCAACCGCCCCCTCAGCAGCGACG |
| | ACAAGGGCGACGGCACCCCCGGCGCCGAACTGTGCAGCACCAACGGCTA |
| | CGCCACCGGCACCAACCCGATGATGCTGAACATCCTCGCCGAGCGGACG |
| | GTCACCAAGGGCGCCTGCGGCACCCCCGTGACCTCGGCCAACACCGTCTC |
| | CTCCGCCCGCACCCTCTACGACGGCAAGCCCTACGGCCAGGCCGGCGAC |
| | CTCGCCGAGTCCACCAGCGCCCTGACCCTGGACCACTACGACACCGGCG |
| | GCAACCCCGTCTACGTCCACACCGCCGCCTCCACCTTCGACGCCTACGGC |
| | CGGCTTACCAGCGTCAGCGAGGCCAACGGCGCCACCTACGACGCCGCGG |
| | GCAACCAGCTCACCGCGCCCAACCTCACCCCCGCCACCACCCGCACCGCC |
| | TACACCCCGGCCACCGGCGCCATCGCCACCACCGTCACCCAGACCACGC |
| | CCACCGGCTGGACCACCACCCTCACCCAGGACCCGGGCCGCGCCGAAGC |
| | TCTGGTCTCCACCGACGCCAACGGCCGCGCCACCACCCAGCAGTACGAC |
| | GGCCTCGGCCGCCTGACCGCCGCCTGGTCACCGGAGCGCGCGACCAACC |
| | TCACCCCCAGCCAGAAGTTCTCCTACGCGGTCAACGGCACCACCGGCCCC |
| | TCCGTCGTCACCTCCCAGTGGCTCAAGGAAGCCGGCGGCTACGCGTACA |
| | AGAACGAGCTGTACGACGGCCTCGGCCGCCTGCGCCAGGTCCAGCGCAC |
| | CAGCGACACCTACTCCGGGCGGCTGATCACCGACACCGTCTACGACTCGC |
| | ACGGCTGGCCCGTCAAGACCGCCAGCCCGTACTACGAGAAGACCACCGC |
| | GCCCAACAGCACCGTCTACCTGCCGCAGGACTCCCAGGTGCCCGCCCAG |
| | ACCTGGGTCACCTTCGACGGCATCGGCCGGACCACCCGCTCCGCGTTCGT |
| | CTCCTACGGACAGCAGCAGTGGGCCACCACCACCGCCTACCCCGGCGCC |
| | GACCGCACCGACGTCACCCCGCCCAACGGCAAATACCCGACCAGCACCT |
| | TCACCGACGGCCGCAACCAGGTCAGCGCCCTGTGGCAGTACCGCACCGC |
| | CACCCCCACCGGCAACCCGGCCGACGCGACCGTCACCACCTACACCTAC |
| | GACGCCGCCAACCGGCCCGCCACCCGCAAGGACGCCGCCGGGAACACCT |
| | GGAGCTACGGCTACGACCTGCGCGGCCGCCAGACCACCGTCACCGACCC |
| | CGACACCGGCACCACCACCACCGCCTACGACGTCAACTCGCGCGCCGTCT |
| | CCACCACCGACGGCAAGGGCAACACCCTCGTCGTCAGCTACGACCTGAT |
| | CGGCCGCAAGACCGGCCTCTACCAGGGCAGCATCGCCCCGGCCAACCAG |
| | CTCGCCGGCTGGACGTACGACACCCTGCCGGGCGGAAAGGGCAAGCCCA |
| | CCTCCTCCACCCGCTACGTCGGGGGCGCCGGCGGCTCGGCCTACACCCAG |
| | GCCGTCACCGGCTACGACGCCGGCTACCGGCCCACCGGCACCTCGGTGA |
| | CGATCCCCGCCAGCGAAGGCAAGCTCGCCGGTACCTACACCACCGGCCT |
| | GACGTACAACCCGGTCCTCGGCACGCTCAAGCAGACCGACCTGCCGGCC |
| | ATCGGCGCGGCGCCCGCCGAGAGCGTCATGTACACCTACAACATCTCCG |
| | GCGTCCTGCAGAAGTCCTACAGCGACACCTACTACGTCTACGACGTGCAG |
| | TACGACGCCTTCGGCCGCCCGGTCCGCACGACCACCGGCGACGCCGGAA |
| | CCCAGGTCGTCTCCACCCAGCTCGACAAGACCGACTACACCTACAACCA |
| | GGCCGGCGACGTCACCTCGGTCACCGACGTCCAGAACGGCACCGCCACC |
| | GACGCCCAGTGCTTCACCTACGACCACCTCGGGCGCCTCACCCAGGCCTG |
| | GACCGACACCGCGGGCTCCACCAGCACCACCAGCGGCACCTGGACCGAC |
| | ACCTCCGGCACCGTCCACAACAGCGGCTCCTCCCAGTCCGTCCCCGCACT |
| | CGGCGCCTGCGCCAACGCCAACGGCCCCGCCAGCACCGGCAGCCCCGCC |
| | AAGCTCTCCGTCGGCGGCCCCTCCCCGTACTGGCAGAGCTACGGCTACGA |
| | CAGCACCGGCAACCGCACCACCCTCGTCCAGCACGACACCACCGGCAAC |
| | ACCACCAAGGACACCACCACCACCCAGACCTTCGGCCCCGCCGGATCGG |
| | TCAACACCGCCACCGGCGCCCCCAACACCGGCGGCGGCACCGGCGGCCC |
| | GCACGCCCTGCTCACCAGCAGCACCACCGGACCCACCGGGACCCAGGTC |
| | ACCAGCTACCAGTACGACCAGCTCGGCAACACCACCGCGGTCACCGAGA |
| | CGTCCGGAACCACCACCCTCGCCTGGAACGGCGAGGACAAGCTCGCCTC |
| | CGTCACCAAGACCGGCCAGGCCCAGGCCACCAGCTACCTCTACGACGCC |
| | GACGGCAACCAGCTCATCCGCCGCAACCCCGGCAAGACCACCCTCAACC |
| | TCGGCAGCGACGAGGTCACCCTCGACACCGCCGCCAACTCCCTCACCGA |
| | CACCCGCTACTACAGCGCCCCCGGCGGCATCAGCATCGCCCGCACCACC |
| | GGACCCACCGGCGCAAGCGCCCTCGCCTACCAGGCCTCCGACCCCCACG |
| | GCACCGCCAACGTCCAGATCAACGTCGACGCCGCCCAGACCACCACCCG |
| | CCGCCCCACCGACCCCTTCGGCAACCCCCGCGGCACCCAGCCCGCCCCCA |
| | ACACCTGGGCCGGCGACAAGGGCTTCGTCGGCGGCACCAAGGACGACAC |
| | CACCGGACTCACCAACCTCGGCGCCCGCGAATACCAACCCACCACCGGC |
| | CGCTTCCTCAACCCCGACCCACTCCTCGACGCCGGCAACCCCCAGCAGTG |
| | GAACGGCTACGCCTACAGCGACAACGACCCCGTCAACAGCTCCGACCCC |
| | AGCGGACTCATCACCAACGCCCTGGCCGACGGCGACACCTACGTCGCCC |
| | GCCCCGCCGCCTTCTGCGTCACCATGTCGTGCGTCGAGCAGACCAGCGGC |
| | CCCGGTTTCTGGGAGGACAAGCGCGTCGGTGACGCCGTCTTCGCCGCCGT |
| | CGTCCAGGCCACCACGCAGAGCAACGGCAACGGGTCATCCCAGACCAAG |
| | AAAGAGAAGGGCATCTGGGGCCAGGCCTGGGACTGGACCAAGAAGAAC |
| | GGCGGCGCCATCCTCGGAGCGCTGGTAGAGGGAGCGGTCTTCAGCACAT |
| | GCTTCATCGGAGCTGGATTCGCCGCACCTGCAACGGGAGGAATCACCGT |
| | CATCGCCGGTGCTGCGGCCTGCGGGGCTGTGGCCGGCGAGGCAGGGGCA |
| | CTGACCACCAATATCCTCACCCCAGATGCCGACCACTCCGTCGACGGCAT |
| | CACCAACGACATGGTCGTTGGTGAAATCACCGGGGCGGCTGTCAGCGCA |
| | GCGAGCGAGGGCGCAAGCTCCCTCGCCAAGCCGGCGGTCCGCAAACTCC |
| | CCGGACCTTGCAACAGTTTCCCGGCCGGCGTCACCGTCCTCCTCGCCGAC |
| | GGCACCACCAAGCCCATCGAACAGATCGCCCAGGGCGACCAGGTAACCG |
| | CCACCGACCCGCAGACAGGCACCACCCAGGCAGAACCCGTCACCGACAC |
| | GATCATCGGCCACGACGACACGGAATTCACCGACCTCACCCTCACCAAC |
| | GACGCAGACCCCCGCGCCCCGCCCAGCGAGATCACCTCCACCACCCACC |
| | ACCCCTACTGGAACGCCACCACCAGCCGCTGGACCGATGCCGGCGACCT |
| | CAAGCCCGGCGACCACGTCCGCACCCCCGACGGCACCGAACTGACCGTC |
| | AACACCGTCTACAGCTACACCACACAACCCCGGACCGCGCGCAACCTCA |
| | CCGTCGCAGACCTCCACACGTACTATGTGCTCGCTGGAAATACGCCGGTC |
| | CTAGTGCATAACACCGGCCCGGGATGTGGTGAGCCGGGATTCGTTAGTG |
| | ACGCTGCTAATTCTCTCTCGGGCAGGCGCATCACCACGGGACAAATATTT |
| | GATGCGAGCGGGAATCCGATCGGGCCTGAGATCACGAGCGGCGGCGGCA |
| | GTCTGGCAGATAGGGCGCAGAGTTATCTTGCCGACTCCCCTAATATTCGA |
| | AATCTGCCCGCTAAGGCGAGATATGCGTCGGCTGACCACGTTGAGGCGC |
| | AATATGCAGTGTGGATGCGAGAAAATGGAGTGACCGACGCCAGTGTGGT |
| | CATCAATCAAAACTATGTATGTGGGCTGCCCCTAGGCTGCCAGGCGGCG |
| | GTGCCCGCTATCCTCCCTCGCGGCTCGACCATGACGGTATGGTATCCAGG |
| | GTCAGGAAGTCCCATCGTATTGCGGGGAGTGGGTTAA |
| | (SEQ ID NO: 372) |
| DddA | >ATE59819.1 type IV secretion protein Rhs |
| homolog in | [Thauera sp. K11] |
| Thauera sp. | MRAFRLIACLLAFSAAAAPAAADTSSMLGRLPEASARQLKERLAPRGLASA |
| K11 | AALRQYLDASQRELDTAPEADDVPARSQRFAARAGELTALREQARRDLASL |
| PROTEIN | EDAAKASGSAEATQRIGRIRGQVDARFDRLEGLFTTWRNAPQGSERRQARR |
| | ELRAALATLRHAGTPAPAAIPVPTLGPLQPAGEPAANPPAARLPAYAQADDA |
| | TGDPFTPGGFRLMKVAALPPAVAAEAATDCSATSADLADDGKDVRLTQPIR |
| | DLAASLDYSPARILRWTQQNVAFEPYWGALKGAEGVLQTRAGNSTDQASL |
| | LIALLRASNIPARYVRGTVQLNDTAAQDDAGGRAQRWLGTKRYRASAAVL |
| | AGGGTSAGLQSIDGTVRGIRFSHVWVQACVPHGAYRGARAEAGGYRWLAL |
| | DAAVKDHDYQQGIAVDVPLTDAAFYTPYLAARSDQLPHEHFAQKVAEAAR |
| | ATDANAALADVPYAGTPRPLRYDVLPGSLPYEVEAFTNWPGLGSSETASLP |
| | DAHRHTFTVTVRNGATTLASAALPYPQNAFKRVTLSYQPTAASQAAWNAW |
| | TGDLPAAADGSIQVVPQIKADGTVLAAGAPANALPLAGVHNVILKVSQGER |
| | SGAACINDSGNPADPKDTDGTCLNKTVYTNIKAGAYHALGLNALHTSNAFL |
| | GQRLEALAAGVQAYPVAPTPAAGAGYEATVGELLHLVLQDYLHQTEQADQ |
| | RNAALRGFKSVGPYDLGLTASDLETDYLFDIPVAIKPAGVFVDFKGGLYGFV |
| | KLDTTAETAAARAAENVDLAKLSIYSGSALEHHVWQQALRTDAVSTVRGL |
| | QFAAEQGIPLVTFTAANIGQYDSLMQMSGATSMAAYKSAIQNAVKGSDNGN |
| | HGVVTVPRAQIAYADPVDPASKWTGAVYMSQNPVTGEYGAIINGTIAGGFP |
| | LLNSTPFSNLYNFDSFVPNTLLGTNGGAGAVQTLPGGTQGESSWITKAGDPV |
| | NMLTGNYTLQARDFTIKGRGGLPIVLERWFNAQNATDGPFGFGWTHSFNHQ |
| | LRFYGIESGQSKVGWVDGTGAQRFYAVAAAGSIAPGTTLAAQAGVFTTLSR |
| | LADGRFQVRETNGLTYSFESLTSPTTPPAAGSEPRARLLAIADRHGNTLTLNY |
| | SGSQLASVSDSLGRTVLSFTWNGNRIGKVKDVSGREVNYAYEDGNGNLTRV |
| | TDPLGQATRYSYYTSADGAKLDHALRRHTLPRGNGMEFEYYAGGQVFRHT |
| | PFDTSGNLIPESALTFHYNSYRRESWTVDGRGAEERFLFDTHGNVIQQTAAN |
| | GATHTYAYADPNDPHLRTRMTDPVGRVTQYSYTAEGYLQTLTLPSGAVQA |
| | WRDYDAFGQPRRVKDARGNWTLHHYDTAGTRTDSIRVKSGVVPTVGTAPA |
| | AANVVSWIKYQGDSVGNLTGVKRLRDWTGATLGNFASGSGPVVTTTFDAA |
| | RLNVASVGRSGNRNGSQISETSPIFSHDALGRLTGGVDGRWYPVAFDYDVL |
| | DRVTRATDATGQPRRYAFDVNGNRIGTELIAGGSRIDSSVAAFDVQDRVAH |
| | VLDHAGNRVAYAYDAVGNRVSVESPDGYAIGFDYDLAGRPYSAYDEDGNR |
| | VFSAFDVAGRVRAVIDPNGAATLYDYHGDEQDGRLARVEQPAIPGQNAGR |
| | AAETDYDAGGLPIRVRQVSAGGEAREGYRFHDELGRVVRSVSAPDDVGQRL |
| | QVCYSYDALSNLTQVRAGATTDTTSAACAGSPAVQLTQSWDDFGNLLTRT |
| | DALGRVWKFEYDAHGNLVASQTPEQAKVSTRSTYRYDPALHGLLAGRSVP |
| | GSGSAGQSVSYARNALGQVIRAETRDGAGNLVVAYDYQYDAAHRVVRIVD |
| | SRGGKALDYAWTPAGRLASITLDGHVWRFQYDGVGRLAAIVAPNGATIAM |
| | ARDAAGRLTERRWPDGAKSAFDWLPEGSLAAIEHSAGGSALAQFAYAYDA |
| | WGNRTSATETLAGTSRSLAYGYDALDRLKTVTTDGATETHAFDLFGNRTSK |
| | TTGGVTTDYLFDAAHQLTQVQIAGTPTERLAYDDNGNLRKHCVGSPSGSTS |
| | DCTGTTVLSLAWNGLDQLIQAARTGLPAESYAYDDAGRRVTKAVGSSATHF |
| | AYDGPDILAEYASPAGSPTAVYAHGAGIDEPLLRLTGATSTPAASAHHYAQD |
| | GLGSIVAAYGEIGASGPVSAASVSATHSYSAGSYPPAKLIDGETTGSTGFWA |
| | GSSGNFAADPAVITLELGAEKSVSRVRLHRVASYLPDYVVKDAEVQVRKPD |
| | NSWQTVGTLTNNTSEDSPEIVLTGAPGSALRVLVKGVRNGSLVLMAEVTMS |
| | ADGGAASVATARYDAWGNVTQASGSIPAFGYTGREPDATGLVYYRARYYH |
| | PALGRFASRDPLGLAAGINPYAYAGGNPILYNDPDGLLAQLAWNTAASYWG |
| | QPIVQETVATIRNGAAVAAGNFVPDTVNGATGWFEQFLHQESGSFGRMDSW |
| | VDVRNPVAQDVAQDLRGVAAVGLMMTPLRYGRASNASFNPPVANLPLNTG |
| | GKTSGMLHIPGQESLSLTSGIAGPSQVVRGQGLPGFNGNQLTHVEGHAAAY |
| | MRTHKVSEAVLDINKAPCTAGSGGGCNGLLPRMLPEGAHLTIRHPNGVQVY |
| | IGTPD (SEQ ID NO: 373) |
| DddA | >CCZ27_07525 NZ_CP023439.1:1708666-1716450 Thauera |
| homolog in | sp. K11 chromosome, complete genome |
| Thauera sp. | ATGCGTGCCTTCCGCCTGATCGCCTGCCTTCTCGCCTTTTCGGCGGCAGCC |
| K11 | GCACCTGCTGCGGCTGACACGTCGTCGATGCTGGGGCGTCTGCCTGAAGC |
| DNA | AAGCGCCCGCCAGCTCAAGGAGCGGTTGGCGCCGCGTGGCCTTGCCTCC |
| | GCTGCCGCCTTGCGCCAGTACCTGGACGCCTCGCAACGCGAGCTGGACA |
| | CCGCACCGGAAGCGGACGACGTACCCGCCCGCAGCCAACGCTTTGCCGC |
| | AAGGGCGGGCGAACTCACCGCGCTGCGCGAACAGGCGCGCCGGGATCTC |
| | GCCAGTCTGGAGGACGCCGCGAAGGCGAGCGGCTCGGCCGAGGCGACGC |
| | AGCGCATCGGTCGAATCCGCGGGCAGGTGGACGCACGCTTCGACCGGCT |
| | CGAAGGGCTTTTTACCACTTGGCGCAATGCGCCCCAGGGCAGCGAACGC |
| | CGCCAGGCCCGCCGCGAACTGCGTGCCGCGCTCGCCACGCTCCGCCATGC |
| | CGGCACCCCGGCTCCGGCTGCGATTCCTGTTCCTACCCTCGGCCCCCTGC |
| | AACCGGCCGGCGAGCCGGCTGCCAACCCACCGGCCGCGCGCTTGCCAGC |
| | CTATGCGCAAGCGGATGACGCGACTGGCGACCCCTTTACCCCCGGTGGCT |
| | TCCGGCTGATGAAGGTCGCCGCACTGCCGCCGGCGGTCGCGGCCGAGGC |
| | GGCAACGGACTGCTCCGCCACCAGCGCCGACCTGGCCGACGACGGCAAG |
| | GACGTGCGCCTGACCCAGCCGATCCGCGACCTCGCGGCATCGCTCGACTA |
| | CTCACCGGCACGCATCCTGCGCTGGACGCAGCAGAACGTCGCCTTCGAA |
| | CCCTACTGGGGGGCACTCAAGGGGGCGGAAGGCGTGCTGCAGACGCGCG |
| | CCGGCAACAGCACCGACCAGGCCAGCCTGCTGATCGCACTCTTGCGGGC |
| | CTCCAACATTCCCGCCCGCTACGTACGCGGCACCGTGCAGCTCAACGACA |
| | CTGCCGCGCAGGACGACGCAGGCGGGCGGGCGCAGCGCTGGCTGGGCAC |
| | CAAGCGCTACCGTGCATCGGCCGCGGTACTCGCCGGCGGCGGAACTTCC |
| | GCCGGCCTGCAGTCGATCGACGGCACCGTCCGCGGCATCCGCTTCAGCCA |
| | TGTCTGGGTCCAGGCCTGCGTTCCCCATGGCGCTTACCGCGGTGCCCGCG |
| | CGGAAGCCGGCGGCTATCGCTGGCTGGCGCTGGACGCGGCGGTGAAGGA |
| | CCATGACTACCAGCAGGGCATCGCGGTCGATGTGCCGCTCACCGATGCC |
| | GCGTTCTACACGCCCTATCTGGCGGCGCGCAGCGACCAGTTGCCGCACGA |
| | GCATTTCGCACAGAAGGTGGCGGAGGCGGCGCGTGCGACCGACGCCAAT |
| | GCGGCGCTGGCCGACGTGCCCTACGCCGGTACGCCGCGGCCGCTGCGCT |
| | ACGACGTGCTGCCCGGTTCGCTGCCCTACGAGGTCGAAGCCTTCACCAAC |
| | TGGCCCGGCCTCGGTTCGTCCGAAACCGCAAGCCTGCCGGACGCACACC |
| | GCCACACCTTCACCGTGACGGTCAGGAACGGCGCCACCACGTTGGCGAG |
| | CGCCGCGCTGCCCTATCCGCAGAACGCCTTCAAGCGCGTCACGCTGTCCT |
| | ATCAGCCGACTGCCGCCTCGCAGGCGGCCTGGAACGCCTGGACGGGCGA |
| | TCTGCCCGCCGCGGCCGACGGCAGCATCCAGGTCGTGCCGCAGATCAAG |
| | GCCGACGGTACCGTGCTCGCCGCAGGTGCGCCCGCCAACGCGCTGCCGC |
| | TCGCCGGCGTGCACAACGTCATCCTCAAGGTCTCGCAGGGCGAGCGCAG |
| | CGGTGCCGCGTGCATCAACGACAGCGGCAACCCCGCCGACCCGAAGGAC |
| | ACCGACGGCACCTGCCTCAACAAGACCGTCTACACCAACATCAAGGCCG |
| | GCGCCTACCACGCCCTGGGCCTGAATGCGCTGCACACCTCGAATGCCTTC |
| | CTCGGCCAGCGGCTCGAAGCGCTGGCGGCCGGCGTGCAGGCCTATCCCG |
| | TCGCGCCCACGCCGGCCGCGGGTGCCGGCTACGAGGCCACGGTCGGTGA |
| | ATTGCTGCATCTGGTGCTGCAGGACTACCTGCACCAGACCGAGCAGGCC |
| | GACCAGCGCAACGCCGCGTTGCGCGGCTTCAAGAGCGTGGGGCCGTACG |
| | ACCTCGGGCTGACCGCGTCCGACCTCGAAACCGACTACCTCTTCGACATC |
| | CCGGTCGCGATCAAGCCGGCCGGCGTGTTCGTGGACTTCAAGGGCGGCC |
| | TCTACGGTTTCGTCAAACTCGATACCACGGCCGAGACGGCCGCGGCACG |
| | CGCCGCCGAAAACGTGGATCTGGCCAAGCTCTCGATCTACTCCGGCTCCG |
| | CGCTCGAACACCACGTCTGGCAGCAGGCGCTGCGCACCGATGCGGTGTC |
| | CACCGTGCGTGGGCTGCAGTTCGCCGCCGAGCAGGGCATTCCGCTCGTCA |
| | CCTTCACCGCGGCCAACATCGGCCAGTACGACAGCCTCATGCAGATGAG |
| | CGGCGCCACCAGCATGGCCGCTTACAAGAGCGCGATCCAGAACGCGGTG |
| | AAGGGCTCGGACAACGGCAACCACGGCGTCGTCACCGTGCCGCGCGCCC |
| | AGATCGCCTACGCCGACCCCGTCGATCCGGCGAGCAAATGGACCGGCGC |
| | GGTCTACATGTCTCAGAACCCCGTCACCGGAGAGTACGGGGCGATCATC |
| | AACGGCACCATCGCCGGCGGCTTCCCGCTGCTCAACAGCACGCCCTTCAG |
| | CAATCTCTACAACTTCGATTCCTTCGTGCCCAACACCCTCCTTGGCACGA |
| | ACGGGGGTGCCGGTGCGGTGCAGACCCTGCCCGGCGGCACCCAGGGCGA |
| | GAGTTCCTGGATCACCAAGGCCGGCGACCCGGTGAACATGCTCACCGGC |
| | AACTACACGCTGCAGGCACGCGACTTCACCATCAAGGGCCGGGGCGGAC |
| | TGCCGATCGTGCTGGAGCGCTGGTTCAACGCGCAGAACGCCACCGACGG |
| | GCCGTTCGGCTTCGGCTGGACGCACAGCTTCAACCATCAGTTGCGTTTCT |
| | ACGGCATCGAGAGCGGCCAGTCCAAGGTCGGCTGGGTGGACGGCACTGG |
| | CGCCCAGCGCTTCTACGCCGTGGCCGCCGCCGGCAGCATTGCGCCGGGC |
| | ACGACGCTGGCCGCGCAGGCCGGGGTGTTCACGACGCTGTCGCGTCTGG |
| | CCGACGGCCGCTTCCAGGTGCGCGAGACCAACGGCCTCACCTACAGCTTC |
| | GAATCGCTCACGAGCCCGACCACCCCGCCGGCCGCGGGCAGCGAACCGC |
| | GCGCAAGACTGCTGGCCATCGCCGACCGCCACGGCAACACCCTGACGCT |
| | CAACTACAGCGGCAGCCAGCTTGCCTCGGTGAGCGACAGCCTCGGCCGC |
| | ACGGTGCTCAGCTTCACCTGGAACGGCAACCGCATCGGCAAGGTGAAGG |
| | ACGTCAGCGGACGGGAAGTGAACTACGCCTACGAGGACGGCAACGGCA |
| | ACCTCACGCGCGTCACCGATCCGCTGGGTCAAGCCACGCGCTACAGCTAC |
| | TACACCAGTGCCGACGGTGCCAAGCTCGACCACGCCCTGCGCCGCCACA |
| | CCCTGCCGCGCGGCAACGGCATGGAGTTCGAGTACTACGCCGGTGGCCA |
| | GGTCTTCCGCCACACGCCGTTCGACACCAGCGGCAACCTCATTCCCGAAT |
| | CGGCGCTGACCTTCCACTACAACAGTTATCGGCGCGAGAGCTGGACGGT |
| | CGATGGCCGCGGTGCCGAGGAGCGCTTCCTGTTCGACACGCACGGCAAC |
| | GTGATCCAGCAGACCGCCGCCAACGGTGCCACCCACACCTACGCGTACG |
| | CCGACCCGAACGATCCGCATCTGCGCACGCGCATGACAGACCCGGTCGG |
| | CCGCGTCACCCAGTACAGCTATACCGCCGAAGGCTATCTGCAGACCCTGA |
| | CGCTGCCGTCGGGCGCCGTGCAGGCGTGGCGCGACTACGACGCCTTCGG |
| | CCAGCCCCGCCGCGTCAAGGACGCGCGCGGCAACTGGACGCTCCACCAC |
| | TACGACACCGCCGGGACACGGACCGACTCCATCCGGGTCAAATCGGGCG |
| | TGGTCCCCACCGTCGGCACCGCGCCTGCCGCGGCCAACGTCGTTTCCTGG |
| | ATCAAGTACCAGGGCGACAGCGTGGGCAACCTCACCGGCGTCAAGCGCC |
| | TGCGCGACTGGACGGGCGCGACCCTGGGCAATTTCGCCAGCGGCAGCGG |
| | CCCCGTCGTCACCACCACCTTCGATGCGGCCAGGCTCAACGTCGCCAGCG |
| | TCGGCCGTAGCGGCAACCGCAACGGCAGCCAGATCAGCGAGACCAGCCC |
| | GATCTTCTCCCACGACGCGCTGGGGCGCCTCACCGGCGGGGTGGACGGG |
| | CGCTGGTATCCGGTCGCCTTCGATTACGACGTGCTCGACCGCGTCACCCG |
| | CGCCACCGACGCCACGGGCCAGCCGCGCCGCTACGCGTTCGACGTCAAC |
| | GGCAACCGCATCGGTACGGAGCTGATTGCCGGCGGCAGCCGTATCGATT |
| | CCTCGGTGGCCGCCTTCGACGTGCAGGACCGCGTCGCCCACGTCCTCGAT |
| | CACGCCGGCAACCGCGTGGCCTACGCCTACGATGCGGTGGGCAACCGGG |
| | TGAGCGTGGAAAGCCCCGACGGCTACGCCATCGGCTTCGACTACGACCT |
| | CGCCGGACGGCCCTATTCGGCCTACGACGAAGACGGCAACCGCGTCTTCT |
| | CCGCCTTCGACGTGGCCGGGCGCGTGCGAGCGGTCATCGACCCCAACGG |
| | CGCCGCGACGCTCTACGACTATCACGGCGACGAGCAGGACGGGCGTCTC |
| | GCGCGCGTGGAGCAGCCCGCCATCCCGGGCCAGAACGCGGGCCGCGCCG |
| | CCGAGACCGACTACGATGCGGGTGGGTTGCCCATCCGCGTGCGCCAGGT |
| | CTCGGCCGGCGGCGAAGCGCGCGAAGGCTACCGTTTCCACGACGAGCTT |
| | GGCCGCGTGGTGCGCAGCGTCTCCGCGCCGGACGACGTCGGCCAGCGGC |
| | TGCAGGTCTGCTACAGCTACGATGCACTCTCGAACCTCACCCAGGTGCGC |
| | GCCGGCGCCACCACCGACACCACCAGTGCCGCCTGCGCCGGCAGCCCCG |
| | CGGTGCAGCTCACCCAGAGCTGGGACGACTTTGGCAACCTGCTGACGCG |
| | CACCGACGCGCTGGGCCGGGTGTGGAAGTTCGAGTACGACGCCCACGGC |
| | AACCTCGTCGCCAGCCAGACGCCCGAGCAGGCCAAGGTCTCGACGCGCA |
| | GCACCTACCGCTACGATCCGGCGCTGCACGGCTTGCTGGCCGGGCGCAG |
| | CGTGCCGGGCAGCGGCAGTGCGGGCCAGAGCGTGAGCTATGCGCGCAAC |
| | GCGCTCGGCCAGGTCATCCGCGCCGAGACGCGCGACGGCGCGGGCAACC |
| | TCGTCGTCGCCTACGACTACCAGTACGACGCCGCCCACCGTGTGGTGCGC |
| | ATCGTCGACAGCCGCGGCGGCAAGGCGCTCGACTACGCCTGGACGCCCG |
| | CCGGGCGGCTGGCGAGCATTACCCTGGACGGCCATGTCTGGCGCTTCCAG |
| | TACGACGGCGTCGGCCGGCTCGCCGCGATCGTCGCGCCCAACGGCGCCA |
| | CCATAGCGATGGCACGCGATGCCGCCGGGCGGCTCACCGAGCGGCGCTG |
| | GCCCGACGGCGCGAAGAGCGCCTTCGACTGGCTGCCCGAAGGCAGCCTC |
| | GCCGCCATCGAGCACAGCGCGGGCGGCAGCGCGCTCGCACAGTTCGCCT |
| | ATGCCTACGATGCCTGGGGCAACCGCACGAGCGCCACCGAGACCCTCGC |
| | GGGCACCAGCCGCAGCCTCGCCTACGGCTACGACGCGCTCGACCGCCTG |
| | AAGACCGTCACCACCGACGGTGCGACCGAAACCCATGCCTTCGATCTCTT |
| | CGGCAATCGCACCAGCAAGACCACGGGGGGGTGACCACCGACTATCTC |
| | TTCGACGCGGCGCACCAGCTCACCCAGGTGCAGATCGCCGGCACCCCCA |
| | CCGAGCGGCTCGCCTACGACGACAACGGTAATCTCCGCAAGCACTGCGT |
| | CGGCAGTCCGAGTGGCAGCACCAGCGATTGCACCGGCACCACCGTGCTG |
| | AGCCTCGCCTGGAACGGCCTCGACCAGTTGATCCAGGCCGCCAGGACGG |
| | GCCTGCCCGCCGAGTCCTACGCCTACGACGATGCCGGGCGGCGTGTCACC |
| | AAGGCGGTGGGCAGCAGCGCCACCCACTTCGCCTACGACGGTCCCGACA |
| | TCCTGGCCGAGTACGCCAGCCCGGCCGGCAGCCCCACCGCCGTCTATGCC |
| | CACGGTGCCGGCATCGACGAACCGCTGCTGCGCCTCACCGGCGCGACGA |
| | GCACGCCGGCCGCTTCCGCGCACCACTACGCGCAGGACGGGCTGGGCAG |
| | CATCGTCGCGGCCTATGGCGAGATCGGCGCCAGCGGTCCGGTCAGTGCC |
| | GCGAGCGTATCGGCCACCCACAGTTACAGCGCCGGCAGCTACCCGCCGG |
| | CAAAGCTGATCGACGGCGAGACGACCGGAAGCACCGGGTTCTGGGCTGG |
| | CAGCTCGGGCAACTTCGCTGCCGATCCAGCCGTGATCACGCTGGAACTGG |
| | GTGCGGAGAAAAGCGTGAGCCGCGTGAGGCTGCACCGGGTGGCCAGCTA |
| | CCTGCCCGACTACGTGGTCAAGGATGCCGAGGTGCAGGTCCGAAAACCG |
| | GACAATTCGTGGCAGACGGTCGGCACGCTGACAAACAACACCAGCGAAG |
| | ACAGTCCCGAGATCGTGCTCACCGGCGCCCCCGGCAGCGCGCTGCGCGT |
| | GCTCGTCAAGGGCGTGCGCAACGGCAGCCTGGTGCTGATGGCCGAGGTG |
| | ACGATGAGTGCGGACGGTGGCGCGGCCAGCGTGGCCACCGCCCGCTACG |
| | ACGCCTGGGGCAACGTCACGCAGGCGAGCGGCAGCATCCCGGCCTTCGG |
| | CTACACCGGACGCGAGCCCGATGCCACGGGCCTGGTCTACTACCGCGCC |
| | CGCTACTACCACCCCGCGCTCGGCCGCTTCGCCAGCCGCGACCCGCTGGG |
| | GCTGGCGGCGGGGATCAATCCCTACGCCTACGCGGGCGGCAATCCCATC |
| | CTCTACAACGATCCGGATGGCTTGCTGGCGCAACTGGCGTGGAATACGG |
| | CGGCCAGCTACTGGGGACAGCCGATAGTTCAAGAAACGGTCGCCACGAT |
| | TCGAAATGGGGCCGCAGTGGCCGCTGGCAACTTCGTTCCAGACACGGTC |
| | AACGGTGCAACAGGTTGGTTTGAGCAGTTCCTGCACCAAGAATCGGGCT |
| | CGTTCGGGCGCATGGACTCGTGGGTGGATGTGCGAAACCCCGTTGCGCA |
| | GGACGTAGCCCAGGACCTGCGCGGTGTCGCAGCCGTTGGGTTAATGATG |
| | ACGCCGCTGCGGTATGGTCGTGCCTCCAACGCGTCTTTCAATCCGCCAGT |
| | AGCCAATCTTCCGCTCAACACTGGAGGAAAAACATCTGGCATGTTGCAC |
| | ATTCCAGGGCAAGAATCACTGTCGCTCACGAGCGGAATTGCGGGGCCGT |
| | CTCAAGTCGTTAGAGGTCAAGGTTTGCCAGGATTCAACGGTAATCAGTTG |
| | ACCCATGTGGAAGGTCATGCTGCTGCTTACATGCGGACTCACAAGGTCTC |
| | TGAGGCTGTTCTGGACATAAACAAAGCACCTTGCACCGCTGGTAGTGGTG |
| | GTGGATGTAATGGGTTGCTTCCCCGAATGCTGCCGGAGGGGGCTCATTTA |
| | ACAATTCGACACCCAAATGGTGTTCAAGTTTATATTGGCACTCCTGACTA |
| | A (SEQ ID NO: 374) |
| Chondromyces | >AKT41505.1 type IV secretion protein Rhs |
| crocatus | [Chondromyces crocatus] |
| PROTEIN | MSMSASRSQPAFPFVSASSPRPRRRPPFPRALLLLIAVLLVGACGDAGGPLLW |
| | SSSSQALWEPSPIPPLPPLLCLGPGDGPSPFPPDLTQGTTTAAGTLPGSFSVTST |
| | GEATYTIPVPTLPGRAGIEPSLAITYDSAQGEGLLGIGFHLQGLSSVDRCPRNV |
| | AQDGHIAPVRDAEDDALCLDGQRLVPVDPQPGRAPREYRTFPDSFTRVEAD |
| | FAESEGWPAERGPKRLRAHGKAGLIYEYGGESSGRVLAQGEAVRSWLLTRL |
| | SDRDGNTMAVVYRNDLHAKGYTVEHAPQRITYTRHPTVPASRMVEFTYGP |
| | LEAADVRVHYARGMELRRSLSLRSIQMFGPGHVLARELRFGYGHGPATGRL |
| | RLEAVRECAGDGTCKPPTRFTWHTAGAAGYTQQQTLVEVPLSERGTLMTM |
| | DVSGDGLDDLVTSDMVVEAGTEEPITRWSVALNRSQELTPGFFEAAVTGQE |
| | QPHFIDAEPPYQPELGTPLDYDHDGRMDLFLHDVHGQSMTWEVLLSNGDGR |
| | FTRRDTGVPRPFTMGMTPAGLRSPDASTHLVDVDGDGMVDLLQCYLSAHE |
| | QLWYLHRWTAAAGGFAPHGDRVHALSSYPCHAELHAVDVDADGRVDLVM |
| | QELILVGSQVRAGWQYVAFSYELSDGSWTRALTGLRLTPPGDRVFFLDVNG |
| | DGLPDAVQSSRDDEQLYTSMNIGAGFAAPVPSLATPTLGAARFVRFASVLDH |
| | NADGRQDLLLAMSDGGSESLPAWKVLQATGEVGPGTFEIVHPGLPMGIVLQ |
| | QDELPTPDHPLTPRVTDVNGDGAQDLLYAFNNQVHVFENVLGQEDLLAAV |
| | TDGMNAHAPEDAEYLPNVQIRYDHLIDRARTTEGFEDAPGIPSPEQRTYRPL |
| | EQSDEEPCRYPVRCVVGHRRVVSGYVLNNGADRPRTFQVAYRNGRHHRLG |
| | RGFLGFGTRIVRDLDTGAGTAEFYDNVTFDGAFQAFPFRGQVQRSWRWSPS |
| | LPLDAHSAEPASLELLTTRSYAVVIPTQAGTYFTLSLLEGKSRHQGTFSPGSG |
| | KTLEEAVRALEGDLASRMSDTLRTVSDFDLYGNILAEQTQTEGVDLDLSVTR |
| | SFDNDPLSWRLGELTRETTCSKAGGETQCRVMHRSYDGRGHVRLERVGGEP |
| | FDPEMQLDVWFSRDALGNIHSTRSRDGTGQVRASCTSYDALGLMPYAHRNL |
| | EGHQSYTRYDPAVGVLRASVDPNGLVSRWAYDGFGRVTLESLPGRMPTVIR |
| | RTWTKDGGAAGNAWNLKIRTASVGGQDETVQLDGLGREVRWWWQALDV |
| | GEEQAPRMMQEVAFDARGEHLAWRSLPIVDPAPPGSVQVRETWQYDGMGR |
| | VLRHVTPWGAATTHEYIGRDEVITAPGQAVTRIASDPLGRPTAVGDPEGGVS |
| | RYTYGPFGGLREVTTPAGAVTLTERDAFGRVRRQVSPDRGVSTAHYDGYGQ |
| | KISSLDAAGRAVTTRYDTLGRIFRQVDEDGVTEFRWDDAQHGVGQLALVVS |
| | PDGHRLRYGFDHLGRPATTTLEIGGESFTSRLSYDLSGRLERIEYPSAPGIGSF |
| | AIEREYDPHGRLRALKDAGSGAEFWRATAIDAGNRITGERFGGGTATTLRTF |
| | DAARERVSRIETQTAGGPVQQLSYLWNDRRKLVERSDGLHANVERFRYDLL |
| | DRLTCAQFGLINAALCERPFTYGPDGNLLQKPGVGAYEYDPAQPHAVVRAG |
| | SAFYGYDAVGNQTSRPGATIAYTAFDLPKRIALTSGDTVDFAYDGLQQRVR |
| | KTTATQEIASFGEVYERVTDVVTGAVEHRYHVRNDERVVTLVRRSVAQGTR |
| | TLHVHVDHLGSIDVLTDGVTGSVAERRSYDAFGAPRHPDWGSGQPPSPHEL |
| | SSLGFTGHEADLDLGLVNMKGRIYDPKLGRFLTPDPLVPRPLFGQSWNSYSY |
| | VLNSPLSLVDPSGFQEQPPATEDGCSQGCTIWVFGPPREPKPPAPPKVVEGNL |
| | EDAAGTGSTQAPVDVGTSGVRSGWSPQLPATLQTLGRGDAIARRIMDGVRI |
| | GMARMLLESAKLGILGGTSRVYVAYTNLTAAWNGYKESGLPGALDAVNPA |
| | SQMVQAGVEAYEAAAAEDWEAAGASLFKAGSIGMSILATAVGVGGAITAT |
| | VGSTAGAAGRAAARAPSLPAYAGGKTSGVLRTTAGDTALLSGYKGPSASMP |
| | RGTPGMNGRIKSHVEAHAAAVMREQGMKEGTLYINRVPCSGATGCDAMLP |
| | RMLPPDAHLRVVGPNGYDQVFVGLPD (SEQ ID NO: 375) |
| Chondromyces | >CMC5_057130 NZ_CP012159.1:7808731-7815414 |
| crocatus | Chondromyces crocatus strain Cm c5, complete genome |
| DNA | ATGTCCATGTCGGCCTCACGGAGTCAGCCCGCATTCCCCTTCGTGTCGGC |
| | CTCCTCTCCGCGTCCGCGCCGGCGCCCTCCCTTTCCCCGAGCGCTGCTCCT |
| | CCTCATCGCCGTGCTCCTCGTCGGCGCATGCGGCGACGCTGGCGGCCCGC |
| | TTCTCTGGTCGAGCAGCTCCCAGGCCCTCTGGGAACCCTCCCCGATCCCG |
| | CCGCTCCCCCCGCTCCTGTGCCTCGGCCCCGGCGACGGTCCCTCCCCCTTT |
| | CCGCCTGACCTTACGCAGGGGACCACCACCGCGGCGGGGACCCTGCCAG |
| | GGAGCTTTTCGGTCACGAGCACGGGCGAGGCGACGTACACGATCCCGGT |
| | CCCCACGCTGCCTGGCCGTGCCGGCATCGAGCCCTCGCTGGCGATCACCT |
| | ACGACAGTGCGCAGGGTGAAGGGCTGCTCGGGATCGGCTTCCACTTGCA |
| | GGGCCTCTCGTCGGTCGATCGCTGCCCCCGGAACGTCGCGCAGGATGGTC |
| | ACATCGCGCCGGTCCGGGATGCCGAGGACGACGCCTTGTGCCTCGATGG |
| | GCAGCGGCTCGTCCCCGTGGACCCGCAGCCAGGGCGTGCGCCGCGGGAA |
| | TACCGCACGTTCCCGGACAGCTTCACGCGCGTCGAGGCCGACTTCGCGGA |
| | GAGCGAGGGGTGGCCGGCGGAGCGTGGGCCGAAGCGGCTGCGGGCGCA |
| | TGGCAAAGCGGGGCTGATCTACGAATACGGTGGAGAATCATCGGGCCGG |
| | GTGCTCGCGCAAGGGGAGGCGGTGCGGTCCTGGTTGCTGACGCGGCTCA |
| | GCGACCGGGATGGCAACACGATGGCGGTGGTCTACCGGAATGACCTCCA |
| | CGCGAAGGGCTACACCGTCGAGCACGCGCCGCAGCGGATCACCTACACC |
| | AGGCACCCGACTGTGCCGGCCTCGCGCATGGTGGAGTTCACGTACGGGC |
| | CGCTGGAGGCGGCGGACGTGCGCGTACACTATGCCCGCGGGATGGAGCT |
| | GCGCCGCTCGCTGAGCTTGCGCTCGATCCAGATGTTCGGGCCGGGACACG |
| | TGCTCGCGAGGGAGCTGCGCTTCGGTTACGGGCATGGGCCGGCGACGGG |
| | TCGCTTGCGACTGGAGGCGGTTCGGGAGTGCGCAGGTGACGGGACGTGC |
| | AAGCCGCCGACACGCTTCACCTGGCACACGGCCGGAGCGGCTGGATACA |
| | CGCAGCAGCAGACACTGGTGGAGGTGCCGCTGTCGGAGCGCGGCACGTT |
| | GATGACGATGGACGTCAGCGGCGATGGCCTCGACGACCTGGTGACGTCC |
| | GACATGGTGGTGGAGGCCGGCACGGAAGAGCCGATCACCCGCTGGTCGG |
| | TCGCGCTCAACCGGAGCCAGGAGCTGACGCCGGGGTTCTTCGAGGCGGC |
| | CGTCACTGGGCAGGAGCAGCCGCATTTCATCGACGCAGAGCCGCCGTAC |
| | CAGCCGGAGCTGGGGACGCCGCTCGACTACGACCACGATGGCCGGATGG |
| | ACCTGTTTCTGCACGATGTGCACGGGCAGTCGATGACGTGGGAGGTGCTG |
| | CTGTCGAATGGAGATGGGCGGTTCACGCGGCGGGATACGGGGGTGCCGC |
| | GGCCGTTCACGATGGGCATGACGCCGGCGGGATTGCGCAGCCCGGATGC |
| | GTCGACCCATCTGGTGGATGTTGACGGTGACGGGATGGTGGACCTGCTGC |
| | AGTGCTACCTGAGCGCGCACGAGCAGCTCTGGTACTTGCACCGCTGGAC |
| | GGCAGCGGCGGGGGGCTTCGCGCCGCACGGCGATCGGGTGCATGCGCTG |
| | AGCTCCTACCCGTGCCACGCCGAGCTGCACGCGGTCGATGTCGACGCGG |
| | ATGGGCGGGTGGACCTGGTGATGCAGGAGCTGATCCTCGTCGGGAGCCA |
| | GGTGCGGGCGGGGTGGCAGTACGTGGCGTTCTCGTACGAGCTGTCCGAT |
| | GGATCGTGGACGCGCGCGCTGACGGGGCTGCGGCTCACGCCGCCTGGGG |
| | ACCGGGTGTTCTTCCTCGACGTCAACGGCGATGGGCTGCCCGATGCGGTG |
| | CAGAGCAGCCGGGACGATGAGCAGCTGTACACGTCGATGAATATCGGCG |
| | CGGGATTCGCGGCGCCGGTACCGAGCCTGGCGACGCCGACGCTCGGGGC |
| | TGCGAGGTTCGTTCGGTTTGCGTCGGTGCTCGATCACAACGCGGATGGGC |
| | GACAAGACCTGCTGCTGGCCATGAGCGATGGGGGATCGGAGTCGCTGCC |
| | CGCGTGGAAGGTGCTCCAGGCGACGGGGGAGGTCGGTCCGGGGACGTTC |
| | GAGATCGTCCATCCCGGGCTGCCGATGGGCATCGTGCTCCAGCAGGACG |
| | AGCTGCCCACGCCCGACCATCCGCTCACGCCGCGGGTCACTGACGTGAAT |
| | GGGGATGGGGCGCAGGATCTGCTCTATGCGTTCAACAACCAGGTCCATG |
| | TGTTCGAGAACGTGCTCGGCCAGGAGGACCTGCTCGCGGCCGTGACCGA |
| | CGGCATGAATGCGCACGCTCCGGAGGACGCCGAGTACCTGCCCAACGTG |
| | CAGATCCGGTACGACCACCTGATCGATCGTGCGCGGACGACGGAGGGCT |
| | TCGAGGATGCTCCAGGGATCCCGTCACCCGAGCAGCGCACCTACCGGCC |
| | TCTGGAGCAAAGCGATGAGGAGCCCTGCCGCTATCCGGTGCGGTGCGTG |
| | GTCGGGCATCGGCGGGTGGTGAGCGGCTATGTGCTCAACAATGGCGCGG |
| | ATCGGCCGCGCACCTTCCAGGTGGCCTACCGCAATGGCCGTCACCATCGC |
| | CTGGGCCGAGGGTTTCTGGGGTTCGGGACGCGGATCGTGCGTGACCTCG |
| | ATACCGGCGCGGGGACGGCCGAGTTCTACGACAACGTCACGTTTGATGG |
| | CGCCTTCCAGGCCTTCCCTTTCCGAGGGCAGGTACAGCGCTCGTGGCGCT |
| | GGAGTCCGAGCTTGCCGCTGGACGCGCATAGCGCGGAGCCGGCGTCCCT |
| | CGAGCTGCTGACGACGCGGAGCTACGCGGTGGTGATCCCCACGCAAGCG |
| | GGGACGTACTTCACCCTCTCGCTGCTGGAGGGCAAGAGCCGTCATCAGG |
| | GCACGTTCTCACCGGGGAGTGGGAAAACGCTCGAAGAAGCCGTGCGCGC |
| | TCTGGAAGGAGATCTCGCCTCGCGAATGAGCGACACGCTCCGCACCGTC |
| | AGCGACTTCGACCTCTACGGGAACATCCTCGCCGAGCAAACGCAGACGG |
| | AGGGCGTCGACCTCGACCTCTCGGTGACGCGCAGCTTCGACAACGACCC |
| | GCTCTCCTGGCGCCTTGGCGAGCTGACGCGAGAGACGACGTGCAGCAAA |
| | GCGGGCGGTGAGACGCAGTGCCGGGTGATGCACCGGAGCTATGACGGGC |
| | GCGGCCACGTTCGCCTGGAGCGCGTCGGGGGAGAGCCCTTCGACCCGGA |
| | GATGCAGCTCGATGTCTGGTTCTCGCGGGACGCGCTGGGCAACATCCACA |
| | GCACCCGGTCACGTGATGGGACGGGGCAGGTGCGCGCGAGCTGCACCAG |
| | CTACGACGCGCTGGGCTTGATGCCTTATGCCCACCGCAACCTGGAGGGCC |
| | ACCAGAGCTATACGCGCTACGACCCGGCCGTGGGCGTGCTGCGGGCGTC |
| | GGTGGATCCCAACGGCCTGGTGAGCCGCTGGGCCTACGATGGCTTCGGG |
| | CGGGTGACGCTGGAGAGCCTCCCCGGGCGCATGCCCACCGTCATCCGGC |
| | GGACCTGGACGAAGGACGGCGGAGCGGCTGGCAACGCCTGGAACCTGA |
| | AGATCCGCACCGCCTCGGTGGGGGGCCAGGACGAGACCGTGCAGCTCGA |
| | TGGTCTCGGGCGGGAGGTGCGCTGGTGGTGGCAAGCGCTCGACGTGGGG |
| | GAAGAGCAAGCGCCGCGGATGATGCAGGAGGTCGCCTTCGATGCGCGGG |
| | GCGAGCACCTCGCGTGGCGCTCGCTGCCGATCGTGGATCCCGCGCCACCA |
| | GGCTCGGTGCAGGTGCGAGAGACGTGGCAATACGACGGGATGGGGCGG |
| | GTGCTCCGGCACGTCACGCCGTGGGGGGCGGCGACGACGCACGAGTACA |
| | TCGGGCGGGACGAGGTCATCACCGCGCCTGGGCAGGCCGTCACCCGAAT |
| | CGCCAGCGATCCGCTCGGGAGGCCCACGGCAGTGGGTGATCCCGAAGGT |
| | GGCGTCAGCCGGTACACCTACGGTCCCTTCGGGGGGCTGCGCGAGGTGA |
| | CCACGCCCGCTGGTGCCGTGACGCTGACCGAGCGGGATGCGTTTGGCCG |
| | CGTGCGACGGCAGGTGAGCCCGGACCGGGGAGTCTCTACTGCGCACTAC |
| | GACGGTTACGGGCAGAAGATCTCATCGCTCGACGCGGCAGGACGCGCGG |
| | TCACGACCCGCTACGACACGCTGGGTCGGATTTTCAGGCAGGTCGACGA |
| | AGACGGCGTCACCGAGTTCCGTTGGGATGACGCGCAGCATGGAGTGGGT |
| | CAGCTCGCGCTGGTGGTCAGCCCCGATGGGCATCGGCTGCGCTACGGCTT |
| | CGACCACCTCGGGCGACCAGCGACGACGACGCTGGAGATCGGAGGGGA |
| | AAGCTTCACCAGCCGGCTGTCTTATGATCTGAGCGGCCGGCTCGAGCGGA |
| | TCGAGTACCCGAGCGCGCCGGGGATTGGCAGCTTCGCCATCGAGCGGGA |
| | GTACGATCCTCACGGGCGGCTGCGGGCGCTGAAGGATGCGGGGTCGGGG |
| | GCGGAGTTCTGGCGAGCCACCGCGATCGATGCGGGGAATCGCATCACGG |
| | GGGAGCGCTTCGGTGGGGGGACCGCCACCACGCTCCGCACGTTCGACGC |
| | GGCACGGGAGCGGGTGAGTCGGATCGAGACGCAGACGGCAGGTGGGCC |
| | CGTCCAGCAGCTCTCCTACCTCTGGAACGATCGCCGCAAGCTCGTCGAGC |
| | GCTCCGATGGCCTCCACGCCAACGTCGAGCGCTTTCGTTACGACCTGCTG |
| | GACCGGCTGACGTGCGCGCAGTTCGGGCTGATCAATGCTGCCCTCTGCGA |
| | GCGACCGTTCACCTACGGACCCGACGGCAACCTGCTCCAGAAGCCCGGC |
| | GTCGGTGCCTACGAGTACGACCCCGCGCAGCCCCACGCCGTCGTCCGAG |
| | CTGGTAGCGCGTTCTACGGCTACGACGCCGTCGGCAACCAGACCTCACG |
| | ACCCGGCGCGACCATCGCCTACACCGCGTTCGACCTACCGAAGCGAATC |
| | GCGCTCACCAGCGGCGACACCGTCGACTTCGCGTACGACGGCCTCCAGC |
| | AGCGGGTGCGCAAGACCACGGCGACGCAGGAGATCGCCTCCTTCGGCGA |
| | GGTGTACGAGCGCGTGACCGATGTCGTCACGGGAGCCGTCGAGCATCGC |
| | TACCACGTGCGCAACGACGAGCGCGTCGTCACGCTGGTGCGGCGCTCGG |
| | TCGCGCAAGGCACGCGCACGCTGCATGTCCATGTCGACCACCTCGGGTCG |
| | ATCGATGTGCTCACCGACGGTGTGACCGGCAGCGTCGCCGAGCGCCGCA |
| | GCTACGATGCCTTCGGCGCACCGCGCCATCCCGACTGGGGTTCGGGTCAG |
| | CCTCCGTCACCCCACGAGCTGTCGTCGCTTGGCTTCACCGGGCACGAGGC |
| | CGACCTCGACCTCGGCCTCGTGAACATGAAGGGGCGCATCTACGACCCC |
| | AAGCTCGGACGGTTCCTCACGCCCGATCCGCTCGTGCCGCGGCCTCTCTT |
| | CGGGCAGAGCTGGAATAGCTATTCGTACGTGCTAAACAGCCCGCTGTCG |
| | CTGGTCGATCCCAGTGGGTTTCAAGAGCAGCCACCTGCGACAGAGGACG |
| | GATGCTCGCAGGGCTGCACCATCTGGGTGTTCGGTCCTCCCCGCGAGCCG |
| | AAGCCACCTGCGCCGCCCAAGGTCGTCGAGGGCAACCTGGAGGACGCCG |
| | CTGGCACTGGTTCGACCCAGGCGCCGGTCGATGTCGGGACCTCCGGGGTC |
| | CGTAGCGGATGGAGTCCGCAGCTCCCGGCCACGTTGCAGACCTTGGGCC |
| | GTGGTGACGCCATCGCCAGGCGCATCATGGACGGCGTCCGCATCGGGAT |
| | GGCCAGGATGCTGCTGGAGTCCGCAAAGCTCGGCATCCTGGGCGGCACC |
| | AGCCGCGTCTACGTCGCCTACACCAACCTCACCGCCGCCTGGAATGGCTA |
| | CAAAGAGAGCGGGCTCCCCGGCGCTCTCGACGCCGTCAATCCCGCCAGC |
| | CAGATGGTCCAAGCCGGCGTGGAGGCCTACGAGGCTGCCGCCGCAGAGG |
| | ACTGGGAGGCCGCCGGCGCCAGCTTGTTCAAGGCCGGGTCGATCGGGAT |
| | GTCGATCCTGGCGACGGCTGTTGGCGTCGGGGGAGCGATCACTGCGACA |
| | GTGGGCTCGACGGCAGGAGCGGCGGGGAGGGCAGCCGCAAGAGCCCCC |
| | TCACTCCCTGCATATGCTGGCGGAAAAACGTCGGGAGTACTACGGACCA |
| | CCGCAGGCGATACAGCACTGCTGAGCGGCTACAAGGGGCCGTCCGCATC |
| | GATGCCTCGAGGAACGCCAGGCATGAACGGACGCATCAAGTCGCATGTA |
| | GAAGCTCATGCGGCTGCCGTGATGCGAGAGCAAGGGATGAAGGAAGGA |
| | ACCCTGTACATCAATCGAGTCCCCTGCTCTGGCGCCACCGGATGCGACGC |
| | GATGCTCCCAAGAATGCTCCCACCAGATGCACACCTTCGCGTGGTCGGTC |
| | CGAATGGTTACGATCAAGTTTTTGTCGGGCTGCCCGACTGA |
| | (SEQ ID NO: 376) |

### Fusion Proteins

In some aspects, the present disclosure provides fusion proteins comprising any of the zinc finger domain-containing proteins provided herein and/or any of the DddA variants provided herein.

In one aspect, the present disclosure provides fusion proteins comprising a zinc finger domain-containing protein disclosed herein and an effector protein. In some embodiments, the effector protein comprises nuclease activity, nickase activity, recombinase activity, deaminase activity, methyltransferase activity, methylase activity, acetylase activity, acetyltransferase activity, transcriptional activation activity, transcriptional repression activity, or polymerase activity. In some embodiments, the effector protein comprises a nucleic acid editing domain. In certain embodiments, the nucleic acid editing domain comprises a deaminase domain (e.g., an adenosine deaminase domain or a cytidine deaminase domain). In certain embodiments, the cytidine deaminase domain is a double-stranded DNA cytidine deaminase (DddA) domain (e.g., a wild type DddA deaminase domain, or any of the DddA variant deaminase domains disclosed herein).

In this aspect, the structure of a fusion protein may comprise, for example:
NH₂-[zinc finger domain-containing protein]-[effector protein]-COOH; or
NH₂-[effector protein]-[zinc finger domain-containing protein]-COOH.

In some embodiments, the structure of a fusion protein comprises:
NH₂-[zinc finger domain-containing protein]-[nuclease]-COOH; or
NH₂-[nuclease]-[zinc finger domain-containing protein]-COOH.

In some embodiments, the structure of a fusion protein comprises:
NH₂-[zinc finger domain-containing protein]-[nickase]-COOH; or
NH₂-[nickase]-[zinc finger domain-containing protein]-COOH.

In some embodiments, the structure of a fusion protein comprises:
NH₂-[zinc finger domain-containing protein]-[recombinase]-COOH; or
NH₂-[recombinase]-[zinc finger domain-containing protein]-COOH.

In some embodiments, the structure of a fusion protein comprises:
NH₂-[zinc finger domain-containing protein]-[deaminase]-COOH; or
NH₂-[deaminase]-[zinc finger domain-containing protein]-COOH.

In some embodiments, the structure of a fusion protein comprises:
NH₂-[zinc finger domain-containing protein]-[methyltransferase]-COOH; or
NH₂-[methyltransferase]-[zinc finger domain-containing protein]-COOH.

In some embodiments, the structure of a fusion protein comprises:
NH₂-[zinc finger domain-containing protein]-[methylase]-COOH; or
NH₂-[methylase]-[zinc finger domain-containing protein]-COOH.

In some embodiments, the structure of a fusion protein comprises:
NH₂-[zinc finger domain-containing protein]-[acetylase]-COOH; or
NH₂-[acetylase]-[zinc finger domain-containing protein]-COOH.

In some embodiments, the structure of a fusion protein comprises:
NH₂-[zinc finger domain-containing protein]-[acetyltransferase]-COOH; or
NH₂-[acetyltransferase]-[zinc finger domain-containing protein]-COOH.

In some embodiments, the structure of a fusion protein comprises:
NH₂-[zinc finger domain-containing protein]-[transcriptional activator]-COOH; or
NH₂-[transcriptional activator]-[zinc finger domain-containing protein]-COOH.

In some embodiments, the structure of a fusion protein comprises:
NH₂-[zinc finger domain-containing protein]-[transcriptional repressor]-COOH; or
NH₂-[transcriptional repressor]-[zinc finger domain-containing protein]-COOH.

In some embodiments, the structure of a fusion protein comprises:
NH₂-[zinc finger domain-containing protein]-[polymerase]-COOH; or
NH₂-[polymerase]-[zinc finger domain-containing protein]-COOH.

In another aspect, the present disclosure provides fusion proteins comprising a programmable DNA binding protein and a first fragment or second fragment of any of the DddA variants disclosed herein. In some embodiments, the programmable DNA binding protein is a nucleic acid-programmable DNA binding protein (napDNAbp), such as a Cas9 protein. In certain embodiments, the napDNAbp is a nickase (e.g., a Cas9 nickase). In certain embodiments, the napDNAbp is a nuclease-inactive napDNAbp (e.g., a dead Cas9). In some embodiments, the napDNAbp is selected from the group consisting of Cas9, Cas12e, Cas12d, Cas12a, Cas12b1, Cas13a, Cas12c, and Argonaute, and optionally has a nickase activity. In some embodiments, the programmable DNA binding protein is a zinc finger protein. In some embodiments, the programmable DNA binding protein is a TALE protein.

In some aspects, the present disclosure provides fusion proteins comprising any of the zinc finger domain-containing proteins disclosed herein fused to a first fragment or a second fragment of any of the DddA variants disclosed herein.

Accordingly, in one aspect, the disclosure relates to a pair of fusion proteins useful for making modifications to the sequence of, for example, genomic DNA and/or mitochondrial DNA (mtDNA). The pair of fusion proteins, in some embodiments, can comprise a first fusion protein comprising a first pDNAbp (e.g., any of the zinc finger domain-containing proteins provided herein) and a first portion or fragment of a DddA, and a second fusion protein comprising a second pDNAbp (e.g., any of the zinc finger domain-containing proteins provided herein) and a second portion or fragment of a DddA, such that the first and the second portions of the DddA reconstitute a DddA upon co-localization in a cell and/or mitochondria. In certain embodiments, that first portion of the DddA is an N-terminal fragment of a DddA and the second portion of the DddA is C-terminal fragment of a DddA. In other embodiments, the first portion of the DddA is a C-terminal fragment of a DddA and the second portion of the DddA is an N-terminal fragment of a DddA. In this aspect, the structure of the pair of fusion proteins can be, for example:
NH₂-[pDNAbp]-[DddA half^{A}]-COOH and NH₂-[pDNAbp]-[DddA half^{B}]-COOH;
NH₂-[DddA-half^{A}]-[pDNAbp]-COOH and NH₂-[DddA-half^{B}]-[pDNAbp]-COOH;
NH₂-[pDNAbp]-[DddA half^{A}]-COOH and NH₂-[DddA-half^{B}]-[pDNAbp]-COOH; or
NH₂-[DddA-half^{A}]-[pDNAbp]-COOH and NH₂-[pDNAbp]-[DddA half^{B}]-COOH,
   wherein “A” or “B” can be the N-terminal or C-terminal half of DddA.

In yet another aspect, the disclosure relates to a pair of fusion proteins useful for making modifications to the sequence of, for example, genomic DNA and/or mitochondrial DNA (mtDNA). The pair of fusion proteins can comprise a first fusion protein comprising a first zinc finger domain-containing protein and a first portion or fragment of a DddA, and a second fusion protein comprising a second zinc finger domain-containing protein and a second portion or fragment of a DddA, such that the first and the second portions of the DddA, upon co-localization in a cell and/or mitochondria, reconstitute an active DddA. In certain embodiments, that first portion of the DddA is an N-terminal fragment of a DddA and the second portion of the DddA is C-terminal fragment of a DddA. In other embodiments, the first portion of the DddA is a C-terminal fragment of a DddA and the second portion of the DddA is an N-terminal fragment of a DddA. In this aspect, the structure of the pair of fusion proteins can be, for example:
NH₂-[zinc finger domain-containing protein]-[DddA half^{A}]-COOH and NH₂-[zinc finger domain-containing protein]-[DddA half^{B}]-COOH;
NH₂-[DddA-half^{A}]-[zinc finger domain-containing protein]-COOH and NH₂-[DddA-half^{B}]-[zinc finger domain-containing protein]-COOH;
NH₂-[zinc finger domain-containing protein]-[DddA half^{A}]-COOH and NH₂-[DddA-half^{B}]-[zinc finger domain-containing protein]-COOH; or
NH₂-[DddA-half^{A}]-[zinc finger domain-containing protein]-COOH and NH₂-[zinc finger domain-containing protein]-[DddA half^{B}]-COOH, wherein “A” or “B” can be the N-terminal or C-terminal half of DddA.

In yet another aspect, the disclosure relates to a pair of fusion proteins useful for making modifications to the sequence of, for example, genomic DNA and/or mitochondrial DNA (mtDNA). The pair of fusion proteins can comprise a first fusion protein comprising a first Cas9 and a first portion or fragment of a DddA, and a second fusion protein comprising a second Cas9 and a second portion or fragment of a DddA, such that the first and the second portions of the DddA, upon co-localization in a cell and/or mitochondria, reconstitute an active DddA. In certain embodiments, that first portion of the DddA is an N-terminal fragment of a DddA (i.e., “DddA halfA”) and the second portion of the DddA is C-terminal fragment of a DddA (i.e., “DddA halfB”). In other embodiments, the first portion of the DddA is a C-terminal fragment of a DddA and the second portion of the DddA is an N-terminal fragment of a DddA. In this aspect, the structure of the pair of fusion proteins can be, for example:
NH₂-[Cas9]-[DddA half^{A}]-COOH and NH₂-[Cas9]-[DddA half^{B}]-COOH;
NH₂-[DddA-half^{A}]-[Cas9]-COOH and NH₂-[DddA-half^{B}]-[Cas9]-COOH;
NH₂-[Cas9]-[DddA half^{A}]-COOH and NH₂-[DddA-half^{B}]-[Cas9]-COOH; or
NH₂-[DddA-half^{A}]-[Cas9]-COOH and [Cas9]-[DddA half^{B}]-COOH, wherein “A” or “B” can be the N-terminal or C-terminal half of DddA.
   Each instance above of “]-[” can be in reference to a linker sequence (e.g., any of the various linker sequences provided herein).

In some embodiments, a first fusion protein comprises a first zinc finger domain-containing protein and a first portion of a DddA variant. In some embodiments, the first portion of the DddA variant comprises an N-terminal truncated DddA. In some embodiments, the first zinc finger domain-containing protein is configured to bind a first nucleic acid sequence proximal to a target nucleotide. In some embodiments, the first portion of a DddA is linked to the remainder of the first fusion protein by the C-terminus of the first portion of a DddA.

In one aspect, the present disclosure provides base editor fusion proteins for use in editing mitochondrial DNA. As used herein, these mitochondrial DNA editor fusion proteins may be referred to as “mtDNA editors” or “mtDNA editing systems.”

In various embodiments, the mtDNA editors described herein comprise (1) a programmable DNA binding protein (“pDNAbp”) (e.g., a zinc finger domain-containing protein, or a CRISPR/Cas9 domain) and a double-stranded DNA deaminase domain, which is capable of carrying out a deamination of a nucleobase at a target site associated with the binding site of the programmable DNA binding protein (pDNAbp).

In some embodiments, the double-stranded DNA deaminase is split into two inactive half portions, with each half portion being fused to a programmable DNA binding protein that binds to a nucleotide sequence either upstream or downstream of a target edit site, and wherein once in the mitochondria, the two half portions (i.e., the N-terminal half and the C-terminal half) reassociate at the target edit site by the co-localization of the programmable DNA binding proteins to binding sites upstream and downstream of the target edit site to be acted on by the DNA deaminase. The reassociation of the two half portions of the double-stranded DNA deaminase restores the deaminase activity at the target edit site. In other embodiments, the double-stranded DNA deaminase can initially be set in an inactive state that can be induced when in the mitochondria. The double-stranded DNA deaminase is preferably delivered initially in an inactive form in order to avoid toxicity inherent with the protein. Any means to regulate the toxic properties of the double-stranded DNA deaminase until such time as the activity is desired to be activated (e.g., in the mitochondria) is contemplated.

### Linkers

In certain embodiments, linkers may be used to link any of the peptides or peptide domains or moieties of the invention (e.g., to link a zinc finger domain-containing protein to a DddA variant).

As defined above, the term “linker,” as used herein, refers to a chemical group or a molecule linking two molecules or moieties (e.g., a binding domain (e.g., a zinc finger domain-containing protein) and an editing domain (e.g., DddA, or portion thereof)). In some embodiments, a linker joins a binding domain (e.g., a zinc finger domain-containing protein) and a catalytic domain (e.g., DddA, or a portion thereof). Typically, the linker is positioned between, or flanked by, two groups, molecules, or other moieties and connected to each one via a covalent bond, thus connecting the two. In some embodiments, the linker is an amino acid or a plurality of amino acids (e.g., a peptide or protein). In some embodiments, the linker is an organic molecule, group, polymer, or chemical moiety. In some embodiments, the linker is 1-100 amino acids in length, for example, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 30-35, 35-40, 40-45, 45-50, 50-60, 60-70, 70-80, 80-90, 90-100, 100-150, or 150-200 amino acids in length. Longer linkers are also contemplated.

The linker may be as simple as a covalent bond, or it may be a polymeric linker many atoms in length. In certain embodiments, the linker is a polypeptide or is otherwise based on amino acids. In other embodiments, the linker is not peptide-like. In certain embodiments, the linker is a covalent bond (e.g., a carbon-carbon bond, disulfide bond, carbon-heteroatom bond, etc.). In certain embodiments, the linker is a carbon-nitrogen bond of an amide linkage. In certain embodiments, the linker is a cyclic or acyclic, substituted or unsubstituted, branched or unbranched aliphatic or heteroaliphatic linker. In certain embodiments, the linker is polymeric (e.g., polyethylene, polyethylene glycol, polyamide, polyester, etc.). In certain embodiments, the linker comprises a monomer, dimer, or polymer of aminoalkanoic acid. In certain embodiments, the linker comprises an aminoalkanoic acid (e.g., glycine, ethanoic acid, alanine, beta-alanine, 3-aminopropanoic acid, 4-aminobutanoic acid, 5-pentanoic acid, etc.). In certain embodiments, the linker comprises a monomer, dimer, or polymer of aminohexanoic acid (Ahx). In certain embodiments, the linker is based on a carbocyclic moiety (e.g., cyclopentane, cyclohexane). In other embodiments, the linker comprises a polyethylene glycol moiety (PEG). In other embodiments, the linker comprises amino acids. In certain embodiments, the linker comprises a peptide. In certain embodiments, the linker comprises an aryl or heteroaryl moiety. In certain embodiments, the linker is based on a phenyl ring. The linker may include functionalized moieties to facilitate attachment of a nucleophile (e.g., thiol, amino) from the peptide to the linker. Any electrophile may be used as part of the linker. Exemplary electrophiles include, but are not limited to, activated esters, activated amides, Michael acceptors, alkyl halides, aryl halides, acyl halides, and isothiocyanates.

In some other embodiments, the linker comprises an amino acid sequence that is greater than one amino acid residue in length. In some embodiments, the linker comprises less than six amino acids in length. In some embodiments, the linker is two amino acid residues in length. In some embodiments, the linker comprises the amino acid sequence of any one of SEQ ID NOs: 202-221.

In some embodiments, a linker comprises the amino acid sequence SGSETPGTSESATPES (SEQ ID NO: 360), which may also be referred to as the XTEN linker. In some embodiments, the linker is 32 amino acids in length. In some embodiments, the linker comprises the amino acid sequence (SGGS)₂-SGSETPGTSESATPES-(SGGS)₂ (SEQ ID NO: 413), which may also be referred to as (SGGS)₂—XTEN-(SGGS)₂ (SEQ ID NO: 413). In some embodiments, the linker comprises the amino acid sequence, wherein n is 0, 1, 2, 3, 4, 5, 6, 7, 8, 9, or 10. In some embodiments, a linker comprises the amino acid sequence SGGS (SEQ ID NO: 322). In some embodiments, a linker comprises (SGGS)ₙ(SEQ ID NO: 414), (GGGS)ₙ (SEQ ID NO: 415), (GGGGS)ₙ (SEQ ID NO: 416), (G)ₙ(SEQ ID NO: 417), (EAAAK)ₙ (SEQ ID NO: 418), (SGGS)ₙ-SGSETPGTSESATPES-(SGGS)ₙ (SEQ ID NO: 419), (GGS)ₙ (SEQ ID NO: 420), SGSETPGTSESATPES (SEQ ID NO: 360), or (XP)ₙ (SEQ ID NO: 421) motif, or a combination of any of these, wherein n is independently an integer between 1 and 30, and wherein X is any amino acid. In some embodiments, n is 1, 2, 3, 4, 5, 6, 7, 8, 9, 10, 11, 12, 13, 14, or 15. In some embodiments, a linker comprises SGSETPGTSESATPES (SEQ ID NO: 360), and SGGS (SEQ ID NO: 322). In some embodiments, a linker comprises SGGSSGSETPGTSESATPESSGGS (SEQ ID NO: 422). In some embodiments, a linker comprises SGGSSGGSSGSETPGTSESATPESSGGSSGGS (SEQ ID NO: 413). In some embodiments, a linker comprises GGSGGSPGSPAGSPTSTEEGTSESATPESGPGTSTEPSEGSAPGSPAGSPTSTEEGTSTEPSE GSAPGTSTEPSEGSAPGTSESATPESGPGSEPATSGGSGGS (SEQ ID NO: 423). In some embodiments, the linker is 24 amino acids in length. In some embodiments, the linker comprises the amino acid sequence SGGSSGGSSGSETPGTSESATPES (SEQ ID NO: 424). In some embodiments, the linker is 40 amino acids in length. In some embodiments, the linker comprises the amino acid sequence SGGSSGGSSGSETPGTSESATPESSGGSSGGSSGGSSGGS (SEQ ID NO: 425). In some embodiments, the linker is 64 amino acids in length. In some embodiments, the linker comprises the amino acid sequence SGGSSGGSSGSETPGTSESATPESSGGSSGGSSGGSSGGSSGSETPGTSESATPESSGGSSG GS (SEQ ID NO: 426). In some embodiments, the linker is 92 amino acids in length. In some embodiments, the linker comprises the amino acid sequence PGSPAGSPTSTEEGTSESATPESGPGTSTEPSEGSAPGSPAGSPTSTEEGTSTEPSEGSAPGT STEPSEGSAPGTSESATPESGPGSEPATS (SEQ ID NO: 427). It should be appreciated that any of the linkers provided herein may be used to link a pDNAbp and a deaminase (e.g., a zinc finger domain-containing protein and a DddA variant); a pDNAbp and an NLS or MTS; or
deaminase and an NLS or MTS.

In some embodiments, any of the fusion proteins provided herein comprise a DddA variant and a zinc finger domain-containing protein that are fused to each other via a linker (e.g., a glycine and serine-rich amino acid linker, optionally wherein the linker is about 13 amino acids in length). In some embodiments, any of the fusion proteins provided herein, comprise an NLS or an MTS, which may be fused to a deaminase (e.g., a DddA variant disclosed herein) or a programmable DNA binding protein (e.g., a zinc finger domain-containing protein disclosed herein). Various linker lengths and flexibilities between a deaminase and a pDNAbp such as a zinc finger protein can be employed (e.g., ranging from very flexible linkers of the form (GGGGS)ₙ (SEQ ID NO: 416) and (G)ₙ(SEQ ID NO: 417) to more rigid linkers of the form (EAAAK)ₙ (SEQ ID NO: 418), (SGGS)ₙ(SEQ ID NO: 414), SGSETPGTSESATPES (SEQ ID NO: 360) (see, e.g., Guilinger J P, Thompson D B, Liu D R. Fusion of catalytically inactive Cas9 to FokI nuclease improves the specificity of genome modification. Nat. Biotechnol. 2014; 32(6): 577-82; the entire contents are incorporated herein by reference) and (XP). (SEQ ID NO: 421)) in order to achieve the optimal length for deaminase activity for the specific application. In some embodiments, n is 1, 2, 3, 4, 5, 6, 7, 8, 9, 10, 11, 12, 13, 14, or 15. In some embodiments, the linker comprises a (GGS)ₙ (SEQ ID NO: 420) motif, wherein n is 1, 3, or 7. In some embodiments, the deaminase and the pDNAbp provided herein are fused via a linker comprising the amino acid sequence SGSETPGTSESATPES (SEQ ID NO: 360), SGGS (SEQ ID NO: 322), SGGSSGSETPGTSESATPESSGGS (SEQ ID NO: 422), SGGSSGGSSGSETPGTSESATPESSGGSSGGS (SEQ ID NO: 413), or GGSGGSPGSPAGSPTSTEEGTSESATPESGPGTSTEPSEGSAPGSPAGSPTSTEEGTSTEPSE GSAPGTSTEPSEGSAPGTSESATPESGPGSEPATSGGSGGS (SEQ ID NO: 323). In some embodiments, the linker is 24 amino acids in length. In some embodiments, the linker comprises the amino acid sequence SGGSSGGSSGSETPGTSESATPES (SEQ ID NO: 424). In some embodiments, the linker is 32 amino acids in length. In some embodiments, the linker is 32 amino acids in length. In some embodiments, the linker comprises the amino acid sequence (SGGS)₂—SGSETPGTSESATPES-(SGGS)₂ (SEQ ID NO: 413), which may also be referred to as (SGGS)₂—XTEN-(SGGS)₂ (SEQ ID NO: 413). In some embodiments, the linker comprises the amino acid sequence, wherein n is 0, 1, 2, 3, 4, 5, 6, 7, 8, 9, or 10. In some embodiments, the linker is 40 amino acids in length. In some embodiments, the linker comprises the amino acid sequence SGGSSGGSSGSETPGTSESATPESSGGSSGGSSGGSSGGS (SEQ ID NO: 425). In some embodiments, the linker is 64 amino acids in length. In some embodiments, the linker comprises the amino acid sequence SGGSSGGSSGSETPGTSESATPESSGGSSGGSSGGSSGGSSGSETPGTSESATPESSGGSSG GS (SEQ ID NO: 426). In some embodiments, the linker is 92 amino acids in length. In some embodiments, the linker comprises the amino acid sequence PGSPAGSPTSTEEGTSESATPESGPGTSTEPSEGSAPGSPAGSPTSTEEGTSTEPSEGSAPGT STEPSEGSAPGTSESATPESGPGSEPATS (SEQ ID NO: 427).

### Uracil Glycosylase Inhibitor (UGI)

In some embodiments, the fusion proteins of the disclosure comprise one or more UGI domains. When the DddA enzyme is employed and deaminates the target nucleotide, it may trigger uracil repair activity in the cell, thereby causing excision of the deaminated nucleotide. This may cause degradation of the nucleic acid or otherwise inhibit the effect of the correction or nucleotide alteration induced by the fusion protein. To inhibit this activity, a UGI may be desired. In some embodiments, a fusion protein comprises more than one UGI. In some embodiments, a fusion protein comprises two UGIs. In some embodiments, a fusion protein contains two UGIs. The UGI or multiple UGIs may be appended or attached to any portion of the fusion protein. In some embodiments, the UGI is attached to the first or second portion of a DddA in the fusion protein. In some embodiments, a second UGI is attached to the first UGI, which is attached to the first or second portion of a DddA in the fusion protein.

In other embodiments, the base editors described herein may comprise one or more uracil glycosylase inhibitors. The term “uracil glycosylase inhibitor” or “UGI,” as used herein, refers to a protein that is capable of inhibiting a uracil-DNA glycosylase base-excision repair enzyme. In some embodiments, a UGI domain comprises a wild-type UGI or a UGI as set forth in SEQ ID NO: 351. In some embodiments, the UGI proteins provided herein include fragments of UGI and proteins homologous to a UGI or a UGI fragment. For example, in some embodiments, a UGI domain comprises a fragment of the amino acid sequence set forth in SEQ ID NO: 351. In some embodiments, a UGI fragment comprises an amino acid sequence that comprises at least 60%, at least 65%, at least 70%, at least 75%, at least 80%, at least 85%, at least 90%, at least 95%, at least 96%, at least 97%, at least 98%, at least 99%, or at least 99.5% of the amino acid sequence as set forth in SEQ ID NO: 351. In some embodiments, a UGI comprises an amino acid sequence homologous to the amino acid sequence set forth in SEQ ID NO: 351, or an amino acid sequence homologous to a fragment of the amino acid sequence set forth in SEQ ID NO: 351. In some embodiments, proteins comprising UGI or fragments of UGI or homologs of UGI or UGI fragments are referred to as “UGI variants.” A UGI variant shares homology to UGI, or a fragment thereof. For example, a UGI variant is at least 70% identical, at least 75% identical, at least 80% identical, at least 85% identical, at least 90% identical, at least 95% identical, at least 96% identical, at least 97% identical, at least 98% identical, at least 99% identical, at least 99.5% identical, or at least 99.9% identical to a wild type UGI or a UGI as set forth in SEQ ID NO: 351. In some embodiments, the UGI variant comprises a fragment of UGI, such that the fragment is at least 70% identical, at least 80% identical, at least 90% identical, at least 95% identical, at least 96% identical, at least 97% identical, at least 98% identical, at least 99% identical, at least 99.5% identical, or at least 99.9% identical to the corresponding fragment of wild-type UGI or a UGI as set forth in SEQ ID NO: 351. In some embodiments, the UGI comprises the following amino acid sequence:

Uracil-DNA glycosylase inhibitor
(>sp|P14739|UNGI_BPPB2)
(SEQ ID NO: 351)
MTNLSDIIEKETGKQLVIQESILMLPEEVEEVIGNKPESDILVHTAYDES
TDENVMLLTSDAPEYKPWALVIQDSNGENKIKML.

The base editors described herein may comprise more than one UGI domain, which may be separated by one or more linkers as described herein. It will also be understood that in the context of the herein disclosed base editors, the UGI domain may be linked to a deaminase domain.

In some embodiments, a UGI is absent from a base editor. In some embodiments, where a base editor comprises a ZFP or mitoZFP, UGIs are removed or are absent from the base editor. In some embodiments, the removal and/or absence of UGIs increases the activity of a DddA.

### NLS Domains

In various embodiments, the fusion proteins described herein may comprise one or more nuclear localization sequences (NLS), which help promote translocation of a protein into the cell nucleus. Such sequences are well-known in the art and can include the following examples:

| | | SEQUENCE |
|---|---|---|
| | | IDENTI- |
| DESCRIPTION | SEQUENCE | FIER |
| NLS OF SV40 | PKKKRKV | 377 |
| LARGE T-AG | | |
| NLS OF POLYOMA | VSRKRPRP | 378 |
| LARGE T-AG | | |
| NLS OF C-MYC | PAAKRVKLD | 379 |
| NLS OF TUS- | KLKIKRPVK | 380 |
| PROTEIN | | |
| NLS OF HEPATITIS | EGAPPAKRAR | 381 |
| D VIRUS ANTIGEN | | |
| NLS OF MURINE | PPQPKKKPLDGE | 382 |
| P53 | | |
| NLS | MKRTADGSEFESPKKKRKV | 383 |
| NLS OF | AVKRPAATKKAGQAKKKKLD | 384 |
| NUCLEOPLASMIN | | |
| NLS OF PE1 AND | SGGSKRTADGSEFEPKKKRKV | 385 |
| PE2 | | |
| NLS OF EGL-13 | MSRRRKANPTKLSENAKKLAKEVEN | 386 |
| NLS | MDSLLMNRRKFLYQFKNVRWAKGRR | 387 |
| | ETYLC | |

The NLS examples above are non-limiting. The PE fusion proteins may comprise any known NLS sequence, including any of those described in Cokol et al., “Finding nuclear localization signals,” EMBO Rep., 2000, 1(5): 411-415 and Freitas et al., “Mechanisms and Signals for the Nuclear Import of Proteins,” Current Genomics, 2009, 10(8): 550-7, each of which are incorporated herein by reference.

### Mitochondrial Targeting Sequence (MTS)

In various embodiments, the DddA variant-containing base editors or the polypeptides that comprise the DddA variant-containing base editors (e.g., the pDNAbps such as ZFPs fused to the DddA variants described herein) may be engineered to include one or more mitochondrial targeting sequences (MTS) (or mitochondrial localization sequence (MLS)) that facilitate the translocation of a polypeptide into the mitochondria. MTS are known in the art, and exemplary sequences are provided herein. In general, MTSs are short peptide sequences (about 3-70 amino acids long) that direct a newly synthesized protein to the mitochondria within a cell. They are usually found at the N-terminus and consist of an alternating pattern of hydrophobic and positively charged amino acids to form what is called an amphipathic helix. Mitochondrial localization sequences can contain additional signals that subsequently target the protein to different regions of the mitochondria, such as the mitochondrial matrix. One exemplary mitochondrial localization sequence is the mitochondrial localization sequence derived from Cox8, a mitochondrial cytochrome c oxidase subunit VIII. In some embodiments, a mitochondrial localization sequence derived from Cox8 includes the amino acid sequence: MSVLTPLLLRGLTGSARRLPVPRAKIHSL (SEQ ID NO: 357). In some embodiments, the mitochondrial localization sequence derived from Cox8 includes an amino acid sequence that is about 50%, 55%, 60%, 65%, 70%, 75%, 80%, 85%, 90% or 95% identical to SEQ ID NO: 357.

### Methods of Treatment

The evolved DddA-containing base editors may be used to deaminate a target base in a double stranded DNA substrate.

The instant disclosure provides methods for the treatment of a subject diagnosed with a disease associated with or caused by a point mutation that can be corrected by the base editors provided herein (e.g., deamination of DNA, including mitochondrial DNA, by a base editor fusion protein). For example, in some embodiments, a method is provided that comprises administering to a subject having such a disease (e.g., MELAS/Leigh syndrome and Leber's hereditary optic neuropathy, or other disorders associated with a point mutation as described herein), an effective amount of a base editor provided herein that corrects the point mutation or introduces a point mutation comprising desired genetic change. In some embodiments, a method is provided that comprises administering to a subject having such a disease, (e.g., MELAS/Leigh syndrome and Leber's hereditary optic neuropathy, other disorders associated with a point mutation as described above), an effective amount of a base editor provided herein (e.g., for deamination of mitochondrial DNA by a base editor fusion protein) that corrects the point mutation or introduces a deactivating mutation into a disease-associated gene. In some embodiments, the disease is a proliferative disease. In some embodiments, the disease is a genetic disease. In some embodiments, the disease is a mitochondrial disease. In some embodiments, the disease is a metabolic disease. In some embodiments, the disease is a lysosomal storage disease. Other diseases that can be treated by correcting a point mutation or introducing a deactivating mutation into a disease-associated gene will be known to those of skill in the art, and the disclosure is not limited in this respect. In some embodiments, the methods comprise editing genes such as MT-TK, Nd1, HBB, or MT-TL1 (e.g., using a fusion protein comprising the architecture of any of the fusion proteins provided in Table 7, Table 8, or Table 31 herein).

The instant disclosure provides methods for the treatment of additional diseases or disorders (e.g., diseases or disorders that are associated with or caused by a point mutation that can be corrected by the base editors provided herein (e.g., through deamination of mitochondrial DNA)). Some such diseases are described herein, and additional suitable diseases that can be treated with the strategies and fusion proteins, or nucleic acids thereof, provided herein will be apparent to those of skill in the art based on the instant disclosure. Exemplary suitable diseases and disorders are listed below. It will be understood that the numbering of the specific positions or residues in the respective sequences depends on the particular protein and numbering scheme used. Numbering might be different (e.g., in precursors of a mature protein and the mature protein itself), and differences in sequences from species to species may affect numbering. One of skill in the art will be able to identify the respective residue in any homologous protein and in the respective encoding nucleic acid by methods well known in the art (e.g., by sequence alignment and determination of homologous residues). Exemplary suitable diseases and disorders include, without limitation: MELAS/Leigh syndrome and Leber's hereditary optic neuropathy.

The base editors described herein may be used to treat any mitochondrial disease or disorder. As used herein, “mitochondrial disorders” related to disorders that are due to abnormal mitochondria such as for example, a mitochondrial genetic mutation, enzyme pathways, etc. Examples of disorders include but are not limited to: loss of motor control, muscle weakness and pain, gastro-intestinal disorders and swallowing difficulties, poor growth, cardiac disease, liver disease, diabetes, respiratory complications, seizures, visual/hearing problems, lactic acidosis, developmental delays, and susceptibility to infection.

The mitochondrial abnormalities give rise to “mitochondrial diseases” that include, but are not limited to: AD: Alzheimer's Disease; ADPD: Alzheimer's Disease and Parkinsons's Disease; AMDF: Ataxia, Myoclonus and Deafness CIPO: Chronic Intestinal Pseudoobstruction with myopathy and Opthalmoplegia; CPEO: Chronic Progressive External Opthalmoplegia; DEAF: Maternally inherited DEAFness or aminoglycoside-induced DEAFness; DEMCHO: Dementia and Chorea; DMDF: Diabetes Mellitus & DeaFness; Exercise Intolerance; ESOC: Epilepsy, Strokes, Optic atrophy, & Cognitive decline; FBSN: Familial Bilateral Striatal Necrosis; FICP: Fatal Infantile Cardiomyopathy Plus, a MELAS-associated cardiomyopathy; GER: Gastrointestinal Reflux; KSS Kearns Sayre Syndrome LDYT: Leber's hereditary optic neuropathy and DYsTonia; LHON: Leber Hereditary Optic Neuropathy; LFMM: Lethal Infantile Mitochondrial Myopathy; MDM: Myopathy and Diabetes Mellitus; MELAS: Mitochondrial Encephalomyopathy, Lactic Acidosis, and Stroke-like episodes; MEPR: Myoclonic Epilepsy and Psychomotor Regression; MERME: MERRF/MELAS overlap disease; MERRF: Myoclonic Epilepsy and Ragged Red Muscle Fibers; MHCM: Maternally Inherited Hypertrophic CardioMyopathy; MICM: Maternally Inherited Cardiomyopathy; MILS: Maternally Inherited Leigh Syndrome; Mitochondrial Encephalocardiomyopathy; Mitochondrial Encephalomyopathy; MM: Mitochondrial Myopathy; MMC: Maternal Myopathy and Cardiomyopathy; Multisystem Mitochondrial Disorder (myopathy, encephalopathy, blindness, hearing loss, peripheral neuropathy); NARP: Neurogenic muscle weakness, Ataxia, and Retinitis Pigmentosa; alternate phenotype at this locus is reported as Leigh Disease; NIDDM: Non-Insulin Dependent Diabetes Mellitus; PEM: Progressive Encephalopathy; PME: Progressive Myoclonus Epilepsy; RTT: Rett Syndrome; and SIDS: Sudden Infant Death Syndrome.

In some embodiments, a mitochondrial disorder that may be treatable using the base editors described herein include Myoclonic Epilepsy with Ragged Red Fibers (MERRF); Mitochondrial Myopathy, Encephalopathy, Lactacidosis, and Stroke (MELAS); Maternally Inherited Diabetes and Deafness (MIDD); Leber's Hereditary Optic Neuropathy (LHON); chronic progressive external ophthalmoplegia (CPEO); Leigh Disease; Kearns-Sayre Syndrome (KSS); Friedreich's Ataxia (FRDA); Co-Enzyme QIO (CoQIO) Deficiency; Complex I Deficiency; Complex II Deficiency; Complex III Deficiency; Complex IV Deficiency; Complex V Deficiency; other myopathies; cardiomyopathy; encephalomyopathy; renal tubular acidosis; neurodegenerative diseases; Parkinson's disease; Alzheimer's disease; amyotrophic lateral sclerosis (ALS); motor neuron diseases; hearing and balance impairments; or other neurological disorders; epilepsy; genetic diseases; Huntington's Disease; mood disorders; nucleoside reverse transcriptase inhibitors (NRTI) treatment; HIV-associated neuropathy; schizophrenia; bipolar disorder; age-associated diseases; cerebral vascular diseases; macular degeneration; diabetes; and cancer.

### Delivery Methods

In another aspect, the present disclosure provides for the delivery of fusion proteins in vitro and in vivo using split DddA protein formulations. The presently disclosed methods for delivering fusion proteins via various methods. In some embodiments, the present disclosure provides AAVs for delivering any of the fusion proteins, polynucleotides, or vectors described herein. For example, DddA proteins have exhibited toxic effects in vivo, and so require special solutions. One such solution is formulating the DddA, and fusion protein thereof, split into pairs that are packaged into two separate rAAV particles that, when co-delivered to a cell, reconstitute the functional DddA protein. Several other special considerations to account for the unique features of fusion protein are described, including the optimization of split sites. MitoTALE-DddA and/or mitoZF-DddA and/or Cas9-DddA fusion proteins, mRNA expressing the fusion proteins, or DNA can be packaged into lipid nanoparticles, rAAV, or lentivirus and injected, ingested, or inhaled to alter genomic DNA in vivo and ex vivo, including for the purposes of establishing animal models of human disease, testing therapeutic and scientific hypotheses in animal models of human disease, and treating disease in humans.

In another aspect, the present disclosure provides for the delivery of base editors, including mtDNA base editors, in vitro and in vivo using various strategies, including on separate vectors using split inteins and as well as direct delivery strategies of the ribonucleoprotein complex (i.e., the base editor complexed to the gRNA and/or the second-site gRNA) using techniques such as electroporation, use of cationic lipid-mediated formulations, and induced endocytosis methods using receptor ligands fused to the ribonucleoprotein complexes. In addition, mRNA delivery methods may also be employed. Any such methods are contemplated herein. The mtDNA BE fusion proteins, or components thereof, preferably be modified with an MTS or other signal sequence that facilitates entry of the mitoZF-DddA (in the case where a pDNAbp is a ZF) or of the polypeptides and the guide RNAs (in the case where a pDNAbp is Cas9) into the mitochondria.

In another aspect, the present disclosure provides for the delivery of base editors in vitro and in vivo using various strategies, including on separate vectors using split inteins and as well as direct delivery strategies of the programmable base editor using techniques such as electroporation, use of cationic lipid-mediated formulations, and induced endocytosis methods using receptor ligands fused to the ribonucleoprotein complexes. Any such methods are contemplated herein.

In some aspects, the invention provides methods comprising delivering one or more base editor-encoding polynucleotides, such as or one or more vectors as described herein encoding one or more components of the base editing system described herein, one or more transcripts thereof, and/or one or proteins transcribed therefrom, to a host cell. In some aspects, the invention further provides cells produced by such methods, and organisms (such as animals, plants, or fungi) comprising or produced from such cells. In some embodiments, a base editor as described herein in combination with (and optionally complexed with) a guide sequence is delivered to a cell. Conventional viral and non-viral based gene transfer methods can be used to introduce nucleic acids in mammalian cells or target tissues. Such methods can be used to administer nucleic acids encoding components of a base editor to cells in culture, or in a host organism. Non-viral vector delivery systems include DNA plasmids, RNA (e.g. a transcript of a vector described herein), naked nucleic acid, and nucleic acid complexed with a delivery vehicle, such as a liposome. Viral vector delivery systems include DNA and RNA viruses, which have either episomal or integrated genomes after delivery to the cell. For a review of gene therapy procedures, see Anderson, Science 256:808-813 (1992); Nabel & Felgner, TIBTECH 11:211-217 (1993); Mitani & Caskey, TIBTECH 11:162-166 (1993); Dillon, TIBTECH 11:167-175 (1993); Miller, Nature 357:455-460 (1992); Van Brunt, Biotechnology 6(10):1149-1154 (1988); Vigne, Restorative Neurology and Neuroscience 8:35-36 (1995); Kremer & Perricaudet, British Medical Bulletin 51(1):31-44 (1995); Haddada et al., in Current Topics in Microbiology and Immunology Doerfler and Bihm (eds) (1995); and Yu et al., Gene Therapy 1:13-26 (1994).

Methods of non-viral delivery of nucleic acids include lipofection, nucleofection, microinjection, biolistics, virosomes, liposomes, immunoliposomes, polycation or lipid:nucleic acid conjugates, naked DNA, artificial virions, and agent-enhanced uptake of DNA. Lipofection is described in e.g., U.S. Pat. Nos. 5,049,386, 4,946,787; and 4,897,355) and lipofection reagents are sold commercially (e.g., Transfectam™ and Lipofectin™). Cationic and neutral lipids that are suitable for efficient receptor-recognition lipofection of polynucleotides include those of Feigner, WO 91/17424; WO 91/16024. Delivery can be to cells (e.g. in vitro or ex vivo administration) or target tissues (e.g. in vivo administration).

The preparation of lipid:nucleic acid complexes, including targeted liposomes such as immunolipid complexes, is well known to one of skill in the art (see, e.g., Crystal, Science 270:404-410 (1995); Blaese et al., Cancer Gene Ther. 2:291-297 (1995); Behr et al., Bioconjugate Chem. 5:382-389 (1994); Remy et al., Bioconjugate Chem. 5:647-654 (1994); Gao et al., Gene Therapy 2:710-722 (1995); Ahmad et al., Cancer Res. 52:4817-4820 (1992); U.S. Pat. Nos. 4,186,183, 4,217,344, 4,235,871, 4,261,975, 4,485,054, 4,501,728, 4,774,085, 4,837,028, and 4,946,787).

The use of RNA or DNA viral based systems for the delivery of nucleic acids take advantage of highly evolved processes for targeting a virus to specific cells in the body and trafficking the viral payload to the nucleus. Viral vectors can be administered directly to patients (in vivo) or they can be used to treat cells in vitro, and the modified cells may optionally be administered to patients (ex vivo). Conventional viral based systems could include retroviral, lentivirus, adenoviral, adeno-associated and herpes simplex virus vectors for gene transfer. Integration in the host genome is possible with the retrovirus, lentivirus, and adeno-associated virus gene transfer methods, often resulting in long term expression of the inserted transgene. Additionally, high transduction efficiencies have been observed in many different cell types and target tissues.

The tropism of a viruses can be altered by incorporating foreign envelope proteins, expanding the potential target population of target cells. Lentiviral vectors are retroviral vectors that are able to transduce or infect non-dividing cells and typically produce high viral titers. Selection of a retroviral gene transfer system would therefore depend on the target tissue. Retroviral vectors are comprised of cis-acting long terminal repeats with packaging capacity for up to 6-10 kb of foreign sequence. The minimum cis-acting LTRs are sufficient for replication and packaging of the vectors, which are then used to integrate the therapeutic gene into the target cell to provide permanent transgene expression. Widely used retroviral vectors include those based upon murine leukemia virus (MuLV), gibbon ape leukemia virus (GaLV), Simian Immuno deficiency virus (SIV), human immuno deficiency virus (HIV), and combinations thereof (see, e.g., Buchscher et al., J. Virol. 66:2731-2739 (1992); Johann et al., J. Virol. 66:1635-1640 (1992); Sommnerfelt et al., Virol. 176:58-59 (1990); Wilson et al., J. Virol. 63:2374-2378 (1989); Miller et al., J. Virol. 65:2220-2224 (1991); PCT/US94/05700). In applications where transient expression is preferred, adenoviral based systems may be used. Adenoviral based vectors are capable of very high transduction efficiency in many cell types and do not require cell division. With such vectors, high titer and levels of expression have been obtained. This vector can be produced in large quantities in a relatively simple system. Adeno-associated virus (“AAV”) vectors may also be used to transduce cells with target nucleic acids, e.g., in the in vitro production of nucleic acids and peptides, and for in vivo and ex vivo gene therapy procedures (see, e.g., West et al., Virology 160:38-47 (1987); U.S. Pat. No. 4,797,368; WO 93/24641; Kotin, Human Gene Therapy 5:793-801 (1994); Muzyczka, J. Clin. Invest. 94:1351 (1994). Construction of recombinant AAV vectors are described in a number of publications, including U.S. Pat. No. 5,173,414; Tratschin et al., Mol. Cell. Biol. 5:3251-3260 (1985); Tratschin, et al., Mol. Cell. Biol. 4:2072-2081 (1984); Hermonat & Muzyczka, PNAS 81:6466-6470 (1984); and Samulski et al., J. Virol. 63:03822-3828 (1989).

Packaging cells are typically used to form virus particles that are capable of infecting a host cell. Such cells include 293 cells, which package adenovirus, and W2 cells or PA317 cells, which package retrovirus. Viral vectors used in gene therapy are usually generated by producing a cell line that packages a nucleic acid vector into a viral particle. The vectors typically contain the minimal viral sequences required for packaging and subsequent integration into a host, other viral sequences being replaced by an expression cassette for the polynucleotide(s) to be expressed. The missing viral functions are typically supplied in trans by the packaging cell line. For example, AAV vectors used in gene therapy typically only possess ITR sequences from the AAV genome which are required for packaging and integration into the host genome. Viral DNA is packaged in a cell line, which contains a helper plasmid encoding the other AAV genes, namely rep and cap, but lacking ITR sequences. The cell line may also be infected with adenovirus as a helper. The helper virus promotes replication of the AAV vector and expression of AAV genes from the helper plasmid. The helper plasmid is not packaged in significant amounts due to a lack of ITR sequences. Contamination with adenovirus can be reduced by, e.g., heat treatment to which adenovirus is more sensitive than AAV. Additional methods for the delivery of nucleic acids to cells are known to those skilled in the art. See, for example, US20030087817, incorporated herein by reference.

In various embodiments, the base editor constructs (including, the split-constructs) may be engineered for delivery in one or more rAAV vectors. An rAAV as related to any of the methods and compositions provided herein may be of any serotype including any derivative or pseudotype (e.g., 1, 2, 3, 4, 5, 6, 7, 8, 9, 10, 11, 12, 13, 2/1, 2/5, 2/8, 2/9, 3/1, 3/5, 3/8, or 3/9). An rAAV may comprise a genetic load (i.e., a recombinant nucleic acid vector that expresses a gene of interest, such as a whole or split base editor fusion protein that is carried by the rAAV into a cell) that is to be delivered to a cell. An rAAV may be chimeric.

As used herein, the serotype of an rAAV refers to the serotype of the capsid proteins of the recombinant virus. Non-limiting examples of derivatives and pseudotypes include rAAV2/1, rAAV2/5, rAAV2/8, rAAV2/9, AAV2-AAV3 hybrid, AAVrh.10, AAVhu.14, AAV3a/3b, AAVrh32.33, AAV-HSC15, AAV-HSC17, AAVhu.37, AAVrh.8, CHt-P6, AAV2.5, AAV6.2, AAV2i8, AAV-HSC15/17, AAVM41, AAV9.45, AAV6(Y445F/Y731F), AAV2.5T, AAV-HAE1/2, AAV clone 32/83, AAVShH10, AAV2 (Y->F), AAV8 (Y733F), AAV2.15, AAV2.4, AAVM41, and AAVr3.45. A non-limiting example of derivatives and pseudotypes that have chimeric VP1 proteins is rAAV2/5-1VP1u, which has the genome of AAV2, capsid backbone of AAV5 and VP1u of AAV1. Other non-limiting example of derivatives and pseudotypes that have chimeric VP1 proteins are rAAV2/5-8VP1u, rAAV2/9-1VP1u, and rAAV2/9-8VP1u.

AAV derivatives/pseudotypes, and methods of producing such derivatives/pseudotypes are known in the art (see, e.g., Mol Ther. 2012 April; 20(4):699-708. doi: 10.1038/mt.2011.287. Epub 2012 Jan. 24. The AAV vector toolkit: poised at the clinical crossroads. Asokan A1, Schaffer D V, Samulski R J.). Methods for producing and using pseudotyped rAAV vectors are known in the art (see, e.g., Duan et al., J. Virol., 75:7662-7671, 2001; Halbert et al., J. Virol., 74:1524-1532, 2000; Zolotukhin et al., Methods, 28:158-167, 2002; and Auricchio et al., Hum. Molec. Genet., 10:3075-3081, 2001).

Methods of making or packaging rAAV particles are known in the art and reagents are commercially available (see, e.g., Zolotukhin et al. Production and purification of serotype 1, 2, and 5 recombinant adeno-associated viral vectors. Methods 28 (2002) 158-167; and U.S. Patent Publication Numbers US20070015238 and US20120322861, which are incorporated herein by reference; and plasmids and kits available from ATCC and Cell Biolabs, Inc.). For example, a plasmid comprising a gene of interest may be combined with one or more helper plasmids, e.g., that contain a rep gene (e.g., encoding Rep78, Rep68, Rep52 and Rep40) and a cap gene (encoding VP1, VP2, and VP3, including a modified VP2 region as described herein), and transfected into a recombinant cells such that the rAAV particle can be packaged and subsequently purified.

Recombinant AAV may comprise a nucleic acid vector, which may comprise at a minimum: (a) one or more heterologous nucleic acid regions comprising a sequence encoding a protein or polypeptide of interest or an RNA of interest (e.g., a siRNA or microRNA), and (b) one or more regions comprising inverted terminal repeat (ITR) sequences (e.g., wild-type ITR sequences or engineered ITR sequences) flanking the one or more nucleic acid regions (e.g., heterologous nucleic acid regions). Herein, heterologous nucleic acid regions comprising a sequence encoding a protein of interest or RNA of interest are referred to as genes of interest.

Any one of the rAAV particles provided herein may have capsid proteins that have amino acids of different serotypes outside of the VP1u region. In some embodiments, the serotype of the backbone of the VP1 protein is different from the serotype of the ITRs and/or the Rep gene. In some embodiments, the serotype of the backbone of the VP1 capsid protein of a particle is the same as the serotype of the ITRs. In some embodiments, the serotype of the backbone of the VP1 capsid protein of a particle is the same as the serotype of the Rep gene. In some embodiments, capsid proteins of rAAV particles comprise amino acid mutations that result in improved transduction efficiency.

In some embodiments, the nucleic acid vector comprises one or more regions comprising a sequence that facilitates expression of the nucleic acid (e.g., the heterologous nucleic acid), e.g., expression control sequences operatively linked to the nucleic acid. Numerous such sequences are known in the art. Non-limiting examples of expression control sequences include promoters, insulators, silencers, response elements, introns, enhancers, initiation sites, termination signals, and poly(A) tails. Any combination of such control sequences is contemplated herein (e.g., a promoter and an enhancer).

Final AAV constructs may incorporate a sequence encoding the gRNA. In other embodiments, the AAV constructs may incorporate a sequence encoding the second-site nicking guide RNA. In still other embodiments, the AAV constructs may incorporate a sequence encoding the second-site nicking guide RNA and a sequence encoding the gRNA.

In various embodiments, programmable base editor fusion proteins can be expressed from appropriate promoters, such as a human U6 (hU6) promoter, a mouse U6 (mU6) promoter, or other appropriate promoter. The programmable base editor fusion proteins can be driven by the same promoters or different promoters.

In some embodiments, a rAAV constructs or the herein compositions are administered to a subject enterally. In some embodiments, a rAAV constructs or the herein compositions are administered to the subject parenterally. In some embodiments, a rAAV particle or the herein compositions are administered to a subject subcutaneously, intraocularly, intravitreally, subretinally, intravenously (IV), intracerebro-ventricularly, intramuscularly, intrathecally (IT), intracisternally, intraperitoneally, via inhalation, topically, or by direct injection to one or more cells, tissues, or organs. In some embodiments, a rAAV particle or the herein compositions are administered to the subject by injection into the hepatic artery or portal vein.

In other aspects, the base editors can be divided at a split site and provided as two halves of a whole/complete base editor. The two halves can be delivered to cells (e.g., as expressed proteins or on separate expression vectors) and once in contact inside the cell, the two halves form the complete base editor through the self-splicing action of the inteins on each base editor half. Split intein sequences can be engineered into each of the halves of the encoded base editor to facilitate their transplicing inside the cell and the concomitant restoration of the complete, functioning base editor.

These split intein-based methods overcome several barriers to in vivo delivery. For example, the DNA encoding base editors is larger than the rAAV packaging limit, and so requires special solutions. One such solution is formulating the editor fused to split intein pairs that are packaged into two separate rAAV particles that, when co-delivered to a cell, reconstitute the functional editor protein. Several other special considerations to account for the unique features of prime editing are described, including the optimization of second-site nicking targets and properly packaging base editors into virus vectors, including lentiviruses and rAAV.

In this aspect, the base editors can be divided at a split site and provided as two halves of a whole/complete base editor. The two halves can be delivered to cells (e.g., as expressed proteins or on separate expression vectors) and once in contact inside the cell, the two halves form the complete base editor through the self-splicing action of the inteins on each base editor half. Split intein sequences can be engineered into each of the halves of the encoded base editor to facilitate their transplicing inside the cell and the concomitant restoration of the complete, functioning base editor.

In various embodiments, the base editors may be engineered as two half proteins (i.e., a BE N-terminal half and a BE C-terminal half) by “splitting” the whole base editor as a “split site.” The “split site” refers to the location of insertion of split intein sequences (i.e., the N intein and the C intein) between two adjacent amino acid residues in the base editor. More specifically, the “split site” refers to the location of dividing the whole base editor into two separate halves, wherein in each halve is fused at the split site to either the N intein or the C intein motifs. The split site can be at any suitable location in the base editor fusion protein, but preferably the split site is located at a position that allows for the formation of two half proteins which are appropriately sized for delivery (e.g., by expression vector) and wherein the inteins, which are fused to each half protein at the split site termini, are available to sufficiently interact with one another when one half protein contacts the other half protein inside the cell.

In some embodiments, the split site is located in the pDNAbp domain. In other embodiments, the split site is located in the double stranded deaminase domain (DddA). In other embodiments, the split site is located in a linker that joins the pDNAbp domain and the double stranded deaminase domain. Preferably, the DddA is split so as to inactivate the deaminase activity until the split fragments are co-localized in the mitochondria a the target site.

In various embodiments, split site design requires finding sites to split and insert an N- and C-terminal intein that are both structurally permissive for purposes of packaging the two half base editor domains into two different AAV genomes. Additionally, intein residues necessary for trans splicing can be incorporated by mutating residues at the N terminus of the C terminal extein or inserting residues that will leave an intein “scar.”

In various embodiments, using SpCas9 nickase (SEQ ID NO: 451, 1368 amino acids) as an example, the split can be between any two amino acids between 1 and 1368. Preferred splits, however, will be located between the central region of the protein, e.g., from amino acids 50-1250, or from 100-1200, or from 150-1150, or from 200-1100, or from 250-1050, or from 300-1000, or from 350-950, or from 400-900, or from 450-850, or from 500-800, or from 550-750, or from 600-700 of SEQ ID NO: 451. In specific exemplary embodiments, the split site may be between 740/741, or 801/802, or 1010/1011, or 1041/1042. In other embodiments the split site may be between 1/2, 2/3, 3/4, 4/5, 5/6, 6/7, 7/8, 8/9, 9/10, 10/11, 12/13, 14/15, 15/16, 17/18, 19/20 . . . 50/51 . . . 100/101 . . . 200/201 . . . 300/301 . . . 400/401 . . . 500/501 . . . 600/601 . . . 700/701 . . . 800/801 . . . 900/901 . . . 1000/1001 . . . 1100/1101 . . . 1200/1201 . . . 1300/1301 . . . and 1367/1368, including all adjacent pairs of amino acid residues.

In various embodiments, the split intein sequences can be engineered by from the following intein sequences.

2-4 INTEIN:
(SEQ ID NO: 388)
CLAEGTRIFDPVTGTTHRIEDVVDGRKPIHVVAAAKDGTLLARPVVSWFDQGTR
DVIGLRIAGGAIVWATPDHKVLTEYGWRAAGELRKGDRVAGPGGSGNSLALSLTADQ
MVSALLDAEPPILYSEYDPTSPFSEASMMGLLTNLADRELVHMINWAKRVPGFVDLTLH
DQAHLLECAWLEILMIGLVWRSMEHPGKLLFAPNLLLDRNQGKCVEGMVEIFDMLLAT
SSRFRMMNLQGEEFVCLKSIILLNSGVYTFLSSTLKSLEEKDHIHRALDKITDTLIHLMAK
AGLTLQQQHQRLAQLLLILSHIRHMSNKGMEHLYSMKYKNVVPLYDLLLEMLDAHRL
HAGGSGASRVQAFADALDDKFLHDMLAEELRYSVIREVLPTRRARTFDLEVEELHTLVA
EGVVVHNC
3-2 INTEIN
(SEQ ID NO: 389)
CLAEGTRIFDPVTGTTHRIEDVVDGRKPIHVVAVAKDGTLLARPVVSWFDQGTR
DVIGLRIAGGAIVWATPDHKVLTEYGWRAAGELRKGDRVAGPGGSGNSLALSLTADQ
MVSALLDAEPPILYSEYDPTSPFSEASMMGLLTNLADRELVHMINWAKRVPGFVDLTLH
DQAHLLECAWLEILMIGLVWRSMEHPGKLLFAPNLLLDRNQGKCVEGMVEIFDMLLAT
SSRFRMMNLQGEEFVCLKSIILLNSGVYTFLSSTLKSLEEKDHIHRALDKITDTLIHLMAK
AGLTLQQQHQRLAQLLLILSHIRHMSNKGMEHLYSMKYTNVVPLYDLLLEMLDAHRLH
AGGSGASRVQAFADALDDKFLHDMLAEELRYSVIREVLPTRRARTFDLEVEELHTLVAE
GVVVHNC
30R3-1 INTEIN
(SEQ ID NO: 390)
CLAEGTRIFDPVTGTTHRIEDVVDGRKPIHVVAAAKDGTLLARPVVSWFDQGTR
DVIGLRIAGGATVWATPDHKVLTEYGWRAAGELRKGDRVAGPGGSGNSLALSLTADQ
MVSALLDAEPPIPYSEYDPTSPFSEASMMGLLTNLADRELVHMINWAKRVPGFVDLTLH
DQAHLLECAWLEILMIGLVWRSMEHPGKLLFAPNLLLDRNQGKCVEGMVEIFDMLLAT
SSRFRMMNLQGEEFVCLKSIILLNSGVYTFLSSTLKSLEEKDHIHRALDKITDTLIHLMAK
AGLTLQQQHQRLAQLLLILSHIRHMSNKGMEHLYSMKYKNVVPLYDLLLEMLDAHRL
HAGGSGASRVQAFADALDDKFLHDMLAEGLRYSVIREVLPTRRARTFDLEVEELHTLV
AEGVVVHNC
30R3-2 INTEIN
(SEQ ID NO: 391)
CLAEGTRIFDPVTGTTHRIEDVVDGRKPIHVVAAAKDGTLLARPVVSWFDQGTR
DVIGLRIAGGATVWATPDHKVLTEYGWRAAGELRKGDRVAGPGGSGNSLALSLTADQ
MVSALLDAEPPILYSEYDPTSPFSEASMMGLLTNLADRELVHMINWAKRVPGFVDLTLH
DQAHLLECAWLEILMIGLVWRSMEHPGKLLFAPNLLLDRNQGKCVEGMVEIFDMLLAT
SSRFRMMNLQGEEFVCLKSIILLNSGVYTFLSSTLKSLEEKDHIHRALDKITDTLIHLMAK
AGLTLQQQHQRLAQLLLILSHIRHMSNKGMEHLYSMKYKNVVPLYDLLLEMLDAHRL
HAGGSGASRVQAFADALDDKFLHDMLAEELRYSVIREVLPTRRARTFDLEVEELHTLVA
EGVVVHNC
30R3-3 INTEIN
(SEQ ID NO: 392)
CLAEGTRIFDPVTGTTHRIEDVVDGRKPIHVVAAAKDGTLLARPVVSWFDQGTR
DVIGLRIAGGATVWATPDHKVLTEYGWRAAGELRKGDRVAGPGGSGNSLALSLTADQ
MVSALLDAEPPIPYSEYDPTSPFSEASMMGLLTNLADRELVHMINWAKRVPGFVDLTLH
DQAHLLECAWLEILMIGLVWRSMEHPGKLLFAPNLLLDRNQGKCVEGMVEIFDMLLAT
SSRFRMMNLQGEEFVCLKSIILLNSGVYTFLSSTLKSLEEKDHIHRALDKITDTLIHLMAK
AGLTLQQQHQRLAQLLLILSHIRHMSNKGMEHLYSMKYKNVVPLYDLLLEMLDAHRL
HAGGSGASRVQAFADALDDKFLHDMLAEELRYSVIREVLPTRRARTFDLEVEELHTLVA
EGVVVHNC
37R3-1 INTEIN
((SEQ ID NO: 393)
CLAEGTRIFDPVTGTTHRIEDVVDGRKPIHVVAAAKDGTLLARPVVSWFDQGTR
DVIGLRIAGGATVWATPDHKVLTEYGWRAAGELRKGDRVAGPGGSGNSLALSLTADQ
MVSALLDAEPPILYSEYNPTSPFSEASMMGLLTNLADRELVHMINWAKRVPGFVDLTLH
DQAHLLERAWLEILMIGLVWRSMEHPGKLLFAPNLLLDRNQGKCVEGMVEIFDMLLAT
SSRFRMMNLQGEEFVCLKSIILLNSGVYTFLSSTLKSLEEKDHIHRALDKITDTLIHLMAK
AGLTLQQQHQRLAQLLLILSHIRHMSNKGMEHLYSMKYKNVVPLYDLLLEMLDAHRL
HAGGSGASRVQAFADALDDKFLHDMLAEGLRYSVIREVLPTRRARTFDLEVEELHTLV
AEGVVVHNC
37R3-2 INTEIN
(SEQ ID NO: 394)
CLAEGTRIFDPVTGTTHRIEDVVDGRKPIHVVAAAKDGTLLARPVVSWFDQGTR
DVIGLRIAGGAIVWATPDHKVLTEYGWRAAGELRKGDRVAGPGGSGNSLALSLTADQ
MVSALLDAEPPILYSEYDPTSPFSEASMMGLLTNLADRELVHMINWAKRVPGFVDLTLH
DQAHLLERAWLEILMIGLVWRSMEHPGKLLFAPNLLLDRNQGKCVEGMVEIFDMLLAT
SSRFRMMNLQGEEFVCLKSIILLNSGVYTFLSSTLKSLEEKDHIHRALDKITDTLIHLMAK
AGLTLQQQHQRLAQLLLILSHIRHMSNKGMEHLYSMKYKNVVPLYDLLLEMLDAHRL
HAGGSGASRVQAFADALDDKFLHDMLAEGLRYSVIREVLPTRRARTFDLEVEELHTLV
AEGVVVHNC
37R3-3 INTEIN
(SEQ ID NO: 395)
CLAEGTRIFDPVTGTTHRIEDVVDGRKPIHVVAVAKDGTLLARPVVSWFDQGTR
DVIGLRIAGGATVWATPDHKVLTEYGWRAAGELRKGDRVAGPGGSGNSLALSLTADQ
MVSALLDAEPPILYSEYDPTSPFSEASMMGLLTNLADRELVHMINWAKRVPGFVDLTLH
DQAHLLERAWLEILMIGLVWRSMEHPGKLLFAPNLLLDRNQGKCVEGMVEIFDMLLAT
SSRFRMMNLQGEEFVCLKSIILLNSGVYTFLSSTLKSLEEKDHIHRALDKITDTLIHLMAK
AGLTLQQQHQRLAQLLLILSHIRHMSNKGMEHLYSMKYKNVVPLYDLLLEMLDAHRL
HAGGSGASRVQAFADALDDKFLHDMLAEELRYSVIREVLPTRRARTFDLEVEELHTLVA
EGVVVHNC

In various embodiments, the split inteins can be used to separately deliver separate portions of a complete Base editor fusion protein to a cell, which upon expression in a cell, become reconstituted as a complete Base editor fusion protein through the trans splicing.

In some embodiments, the disclosure provides a method of delivering a Base editor fusion protein to a cell, comprising: constructing a first expression vector encoding an N-terminal fragment of the Base editor fusion protein fused to a first split intein sequence;
constructing a second expression vector encoding a C-terminal fragment of the Base editor fusion protein fused to a second split intein sequence; delivering the first and second expression vectors to a cell, wherein the N-terminal and C-terminal fragment are reconstituted as the Base editor fusion protein in the cell as a result of trans splicing activity causing self-excision of the first and second split intein sequences.

In other embodiments, the split site is in the pDNAbp domain.

In still other embodiments, the split site is in the deaminase domain.

In yet other embodiments, the split site is in the linker.

In other embodiments, the base editors may be delivered by ribonucleoprotein complexes.

In this aspect, the base editors may be delivered by non-viral delivery strategies involving delivery of a base editor protein or nucleic acids encoding a base editor by various methods, including electroporation and lipid nanoparticles. Methods of non-viral delivery of nucleic acids include lipofection, nucleofection, microinjection, biolistics, virosomes, liposomes, immunoliposomes, polycation or lipid:nucleic acid conjugates, naked DNA, artificial virions, and agent-enhanced uptake of DNA. Lipofection is described in e.g., U.S. Pat. Nos. 5,049,386, 4,946,787; and 4,897,355) and lipofection reagents are sold commercially (e.g., Transfectam™ and Lipofectin™). Cationic and neutral lipids that are suitable for efficient receptor-recognition lipofection of polynucleotides include those of Feigner, WO 91/17424; WO 91/16024. Delivery can be to cells (e.g. in vitro or ex vivo administration) or target tissues (e.g. in vivo administration).

The preparation of lipid:nucleic acid complexes, including targeted liposomes such as immunolipid complexes, is well known to one of skill in the art (see, e.g., Crystal, Science 270:404-410 (1995); Blaese et al., Cancer Gene Ther. 2:291-297 (1995); Behr et al., Bioconjugate Chem. 5:382-389 (1994); Remy et al., Bioconjugate Chem. 5:647-654 (1994); Gao et al., Gene Therapy 2:710-722 (1995); Ahmad et al., Cancer Res. 52:4817-4820 (1992); U.S. Pat. Nos. 4,186,183, 4,217,344, 4,235,871, 4,261,975, 4,485,054, 4,501,728, 4,774,085, 4,837,028, and 4,946,787).

### Pharmaceutical Compositions

Other aspects of the present disclosure relate to pharmaceutical compositions comprising any of the zinc finger protein variants, deaminase variants, and fusion proteins described herein. The term “pharmaceutical composition”, as used herein, refers to a composition formulated for pharmaceutical use. In some embodiments, the pharmaceutical composition further comprises a pharmaceutically acceptable carrier. In some embodiments, the pharmaceutical composition comprises additional agents (e.g., for specific delivery, increasing half-life, or other therapeutic compounds).

As used here, the term “pharmaceutically-acceptable carrier” means a pharmaceutically-acceptable material, composition, or vehicle, such as a liquid or solid filler, diluent, excipient, manufacturing aid (e.g., lubricant, talc magnesium, calcium or zinc stearate, or steric acid), or solvent encapsulating material, involved in carrying or transporting the fusion protein or zinc finer proteins variant or deaminase variant from one site (e.g., the delivery site) of the body, to another site (e.g., organ, tissue, or portion of the body). A pharmaceutically acceptable carrier is “acceptable” in the sense of being compatible with the other ingredients of the formulation and not injurious to the tissue of the subject (e.g., physiologically compatible, sterile, physiologic pH, etc.). Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids; (23) serum component, such as serum albumin, HDL and LDL; (22) C2-C12 alcohols, such as ethanol; and (23) other non-toxic compatible substances employed in pharmaceutical formulations. Wetting agents, coloring agents, release agents, coating agents, sweetening agents, flavoring agents, perfuming agents, preservatives, and antioxidants can also be present in the formulation. The terms such as “excipient”, “carrier”, “pharmaceutically acceptable carrier” or the like are used interchangeably herein.

In some embodiments, the pharmaceutical composition is formulated for delivery to a subject, e.g., for gene editing. Suitable routes of administering the pharmaceutical composition described herein include, without limitation: topical, subcutaneous, transdermal, intradermal, intralesional, intraarticular, intraperitoneal, intravesical, transmucosal, gingival, intradental, intracochlear, transtympanic, intraorgan, epidural, intrathecal, intramuscular, intravenous, intravascular, intraosseus, periocular, intratumoral, intracerebral, and intracerebroventricular administration.

In some embodiments, the pharmaceutical composition described herein is administered locally to a diseased site (e.g., tumor site). In some embodiments, the pharmaceutical composition described herein is administered to a subject by injection, by means of a catheter, by means of a suppository, or by means of an implant, the implant being of a porous, non-porous, or gelatinous material, including a membrane, such as a sialastic membrane, or a fiber.

In other embodiments, the pharmaceutical composition described herein is delivered in a controlled release system. In one embodiment, a pump may be used (see, e.g., Langer, 1990, Science 249:1527-1533; Sefton, 1989, CRC Crit. Ref Biomed. Eng. 14:201; Buchwald et al., 1980, Surgery 88:507; Saudek et al., 1989, N. Engl. J. Med. 321:574). In another embodiment, polymeric materials can be used. (See, e.g., Medical Applications of Controlled Release (Langer and Wise eds., CRC Press, Boca Raton, Fla., 1974); Controlled Drug Bioavailability, Drug Product Design and Performance (Smolen and Ball eds., Wiley, New York, 1984); Ranger and Peppas, 1983, Macromol. Sci. Rev. Macromol. Chem. 23:61. See also Levy et al., 1985, Science 228:190; During et al., 1989, Ann. Neurol. 25:351; Howard et al., 1989, J. Neurosurg. 71:105). Other controlled release systems are discussed, for example, in Langer, supra.

In some embodiments, the pharmaceutical composition is formulated in accordance with routine procedures as a composition adapted for intravenous or subcutaneous administration to a subject, e.g., a human. In some embodiments, pharmaceutical compositions for administration by injection are solutions in sterile isotonic aqueous buffer. Where necessary, the pharmaceutical composition can also include a solubilizing agent and a local anesthetic such as lignocaine to ease pain at the site of the injection. Generally, the ingredients are supplied either separately or mixed together in unit dosage form, for example, as a dry lyophilized powder or water-free concentrate in a hermetically sealed container such as an ampoule or sachette indicating the quantity of active agent. Where the pharmaceutical composition is to be administered by infusion, it can be dispensed with an infusion bottle containing sterile pharmaceutical grade water or saline. Where the pharmaceutical composition is administered by injection, an ampoule of sterile water for injection or saline can be provided so that the ingredients can be mixed prior to administration.

A pharmaceutical composition for systemic administration may be a liquid, e.g., sterile saline, lactated Ringer's, or Hank's solution. In addition, the pharmaceutical composition can be in solid forms and re-dissolved or suspended immediately prior to use. Lyophilized forms are also contemplated.

The pharmaceutical composition can be contained within a lipid particle or vesicle, such as a liposome or microcrystal, which is also suitable for parenteral administration. The particles can be of any suitable structure, such as unilamellar or plurilamellar, so long as compositions are contained therein. Proteins can be entrapped in “stabilized plasmid-lipid particles” (SPLP) containing the fusogenic lipid dioleoylphosphatidylethanolamine (DOPE), low levels (5-10 mol %) of cationic lipid and stabilized by a polyethyleneglycol (PEG) coating (Zhang Y. P. et al., Gene Ther. 1999, 6:1438-47). Positively charged lipids such as N-[1-(2,3-dioleoyloxi)propyl]-N,N,N-trimethyl-amoniummethylsulfate, or “DOTAP,” are particularly preferred for such particles and vesicles. The preparation of such lipid particles is well known. See, e.g., U.S. Pat. Nos. 4,880,635; 4,906,477; 4,911,928; 4,917,951; 4,920,016; and 4,921,757; each of which is incorporated herein by reference.

The pharmaceutical compositions described herein may be administered or packaged as a unit dose, for example. The term “unit dose” when used in reference to a pharmaceutical composition of the present disclosure refers to physically discrete units suitable as unitary dosage for the subject, each unit containing a predetermined quantity of active material calculated to produce the desired therapeutic effect in association with the required diluent; i.e., carrier, or vehicle.

Further, the pharmaceutical composition can be provided as a pharmaceutical kit comprising (a) a container containing a zinc finger protein variant, deaminase variant, and/or fusion protein of the present disclosure in lyophilized form and (b) a second container containing a pharmaceutically acceptable diluent (e.g., sterile water) for injection. The pharmaceutically acceptable diluent can be used for reconstitution or dilution of the lyophilized zinc finger protein variant, deaminase variant, and/or fusion protein of the present disclosure. Optionally associated with such container(s) can be a notice in the form prescribed by a governmental agency regulating the manufacture, use, or sale of pharmaceuticals or biological products, which notice reflects approval by the agency of manufacture, use, or sale for human administration.

In another aspect, an article of manufacture containing materials useful for the treatment of the diseases described above is included. In some embodiments, the article of manufacture comprises a container and a label. Suitable containers include, for example, bottles, vials, syringes, and test tubes. The containers may be formed from a variety of materials such as glass or plastic. In some embodiments, the container holds a composition that is effective for treating a disease and may have a sterile access port. For example, the container may be an intravenous solution bag or a vial having a stopper pierce-able by a hypodermic injection needle. The active agent in the composition is a zinc finger protein variant, deaminase variant, and/or fusion protein of the present disclosure. In some embodiments, the label on or associated with the container indicates that the composition is used for treating the disease of choice. The article of manufacture may further comprise a second container comprising a pharmaceutically-acceptable buffer, such as phosphate-buffered saline, Ringer's solution, or dextrose solution. It may further include other materials desirable from a commercial and user standpoint, including other buffers, diluents, filters, needles, syringes, and package inserts with instructions for use.

### Kits and Cells

The zinc finger protein variants, deaminase variants, fusion proteins, and compositions of the present disclosure may be assembled into kits. In some embodiments, the kit comprises polynucleotides for expression of the zinc finger protein variants, deaminase variants, and/or fusion proteins described herein.

The kits described herein may include one or more containers housing components for performing the methods described herein, and optionally instructions for use. Any of the kits described herein may further comprise components needed for performing the methods described herein. Each component of the kits, where applicable, may be provided in liquid form (e.g., in solution) or in solid form, (e.g., a dry powder). In certain cases, some of the components may be reconstitutable or otherwise processible (e.g., to an active form), for example, by the addition of a suitable solvent or other species (for example, water), which may or may not be provided with the kit.

In some embodiments, the kits may optionally include instructions and/or promotion for use of the components provided. As used herein, “instructions” can define a component of instruction and/or promotion, and typically involve written instructions on or associated with packaging of the disclosure. Instructions also can include any oral or electronic instructions provided in any manner such that a user will clearly recognize that the instructions are to be associated with the kit, for example, audiovisual (e.g., videotape, DVD, etc.), Internet, and/or web-based communications, etc. The written instructions may be in a form prescribed by a governmental agency regulating the manufacture, use, or sale of pharmaceuticals or biological products, which can also reflect approval by the agency of manufacture, use or sale for animal administration. As used herein, “promoted” includes all methods of doing business including methods of education, hospital and other clinical instruction, scientific inquiry, drug discovery or development, academic research, pharmaceutical industry activity including pharmaceutical sales, and any advertising or other promotional activity including written, oral, and electronic communication of any form, associated with the disclosure. Additionally, the kits may include other components depending on the specific application, as described herein.

The kits may contain any one or more of the components described herein in one or more containers. The components may be prepared sterilely, packaged in a syringe, and shipped refrigerated. Alternatively, they may be housed in a vial or other container for storage. A second container may have other components prepared sterilely. Alternatively, the kits may include the active agents premixed and shipped in a vial, tube, or other container.

The kits may have a variety of forms, such as a blister pouch, a shrink-wrapped pouch, a vacuum sealable pouch, a sealable thermoformed tray, or a similar pouch or tray form, with the accessories loosely packed within the pouch, one or more tubes, containers, a box, or a bag. The kits may be sterilized after the accessories are added, thereby allowing the individual accessories in the container to be otherwise unwrapped. The kits can be sterilized using any appropriate sterilization techniques, such as radiation sterilization, heat sterilization, or other sterilization methods known in the art. The kits may also include other components, depending on the specific application, for example, containers, cell media, salts, buffers, reagents, syringes, needles, a fabric, such as gauze, for applying or removing a disinfecting agent, disposable gloves, a support for the agents prior to administration, etc. Some aspects of this disclosure provide kits comprising a nucleic acid construct comprising a nucleotide sequence encoding the zinc finger protein variants, deaminase variants, and/or fusion proteins described herein, or various components or portions thereof. In some embodiments, the nucleotide sequence(s) comprises a heterologous promoter (or more than a single promoter) that drives expression of the protein(s).

Cells that may contain any of the zinc finger protein variants, deaminase variants, fusion proteins, and compositions described herein include prokaryotic cells and eukaryotic cells. The methods described herein may be used to deliver a zinc finger protein variant, deaminase variant, or fusion protein into a eukaryotic cell (e.g., a mammalian cell, such as a human cell). In some embodiments, the cell is in vitro (e.g., cultured cell). In some embodiments, the cell is in vivo (e.g., in a subject such as a human subject). In some embodiments, the cell is ex vivo (e.g., isolated from a subject and may be administered back to the same or a different subject).

Mammalian cells of the present disclosure include human cells, primate cells (e.g., vero cells), rat cells (e.g., GH3 cells, OC23 cells) or mouse cells (e.g., MC3T3 cells). There are a variety of human cell lines, including, without limitation, human embryonic kidney (HEK) cells, HeLa cells, cancer cells from the National Cancer Institute's 60 cancer cell lines (NCI60), DU145 (prostate cancer) cells, Lncap (prostate cancer) cells, MCF-7 (breast cancer) cells, MDA-MB-438 (breast cancer) cells, PC3 (prostate cancer) cells, T47D (breast cancer) cells, THP-1 (acute myeloid leukemia) cells, U87 (glioblastoma) cells, SHSY5Y human neuroblastoma cells (cloned from a myeloma) and Saos-2 (bone cancer) cells. In some embodiments, zinc finger protein variants, deaminase variants, and/or fusion proteins of the present disclosure are delivered into human embryonic kidney (HEK) cells (e.g., HEK293 or HEK293T cells). In some embodiments, zinc finger protein variants, deaminase variants, and/or fusion proteins of the present disclosure are delivered into stem cells (e.g., human stem cells) such as, for example, pluripotent stem cells (e.g., human pluripotent stem cells including human induced pluripotent stem cells (hiPSCs)). A stem cell refers to a cell with the ability to divide for indefinite periods in culture and to give rise to specialized cells. A pluripotent stem cell refers to a type of stem cell that is capable of differentiating into all tissues of an organism, but not alone capable of sustaining full organismal development. A human induced pluripotent stem cell refers to a somatic (e.g., mature or adult) cell that has been reprogrammed to an embryonic stem cell-like state by being forced to express genes and factors important for maintaining the defining properties of embryonic stem cells (see, e.g., Takahashi and Yamanaka, Cell 126 (4): 663-76, 2006, incorporated by reference herein). Human induced pluripotent stem cells express stem cell markers and are capable of generating cells characteristic of all three germ layers (i.e., ectoderm, endoderm, mesoderm).

Additional non-limiting examples of cell lines that may be used in accordance with the present disclosure include 293-T, 293-T, 3T3, 4T1, 721, 9L, A-549, A172, A20, A253, A2780, A2780ADR, A2780cis, A431, ALC, B16, B35, BCP-1, BEAS-2B, bEnd.3, BHK-21, BR 293, BxPC3, C2C12, C3H-10T1/2, C6, C6/36, Cal-27, CGR8, CHO, CML T1, CMT, COR-L23, COR-L23/5010, COR-L23/CPR, COR-L23/R23, COS-7, COV-434, CT26, D17, DH82, DU145, DuCaP, E14Tg2a, EL4, EM2, EM3, EMT6/AR1, EMT6/AR10.0, FM3, H1299, H69, HB54, HB55, HCA2, Hepa1c1c7, High Five cells, HL-60, HMEC, HT-29, HUVEC, J558L cells, Jurkat, JY cells, K562 cells, KCL22, KG1, Ku812, KYO1, LNCap, Ma-Mel 1, 2, 3 . . . 48, MC-38, MCF-10A, MCF-7, MDA-MB-231, MDA-MB-435, MDA-MB-468, MDCK II, MG63, MONO-MAC 6, MOR/0.2R, MRC5, MTD-1A, MyEnd, NALM-1, NCI-H69/CPR, NCI-H69/LX10, NCI-H69/LX20, NCI-H69/LX4, NIH-3T3, NW-145, OPCN/OPCT Peer, PNT-1A/PNT 2, PTK2, Raji, RBL cells, RenCa, RIN-5F, RMA/RMAS, S2, Saos-2 cells, Sf21, Sf9, SiHa, SKBR3, SKOV-3, T-47D, T2, T84, THP1, U373, U87, U937, VCaP, WM39, WT-49, X63, YAC-1, and YAR cells.

Some aspects of this disclosure provide cells comprising any of the constructs disclosed herein. In some embodiments, a host cell is transiently or non-transiently transfected with one or more vectors described herein. In some embodiments, a cell is transfected as it naturally occurs in a subject. In some embodiments, a cell that is transfected is taken from a subject. In some embodiments, the cell is derived from cells taken from a subject, such as a cell line. A wide variety of cell lines for tissue culture are known in the art. Examples of cell lines include, but are not limited to, C8161, CCRF-CEM, MOLT, mIMCD-3, NHDF, HeLa-S3, Huhl, Huh4, Huh7, HUVEC, HASMC, HEKn, HEKa, MiaPaCell, Panc1, PC-3, TF1, CTLL-2, C1R, Rat6, CV1, RPTE, A10, T24, J82, A375, ARH-77, Calu1, SW480, SW620, SKOV3, SK-UT, CaCo2, P388D1, SEM-K2, WEHI-231, HB56, TIB55, Jurkat, J45.01, LRMB, Bcl-1, BC-3, IC21, DLD2, Raw264.7, NRK, NRK-52E, MRC5, MEF, Hep G2, HeLa B, HeLa T4, COS, COS-1, COS-6, COS-M6A, BS-C-1 monkey kidney epithelial, BALB/3T3 mouse embryo fibroblast, 3T3 Swiss, 3T3-L1, 132-d5 human fetal fibroblasts; 10.1 mouse fibroblasts, 293-T, 3T3, 721, 9L, A2780, A2780ADR, A2780cis, A 172, A20, A253, A431, A-549, ALC, B16, B35, BCP-1 cells, BEAS-2B, bEnd.3, BHK-21, BR 293. BxPC3. C3H-10T1/2, C6/36, Cal-27, CHO, CHO-7, CHO-IR, CHO-K1, CHO-K2, CHO-T, CHO Dhfr −/−, COR-L23, COR-L23/CPR, COR-L23/5010, COR-L23/R23, COS-7, COV-434, CML T1, CMT, CT26, D17, DH82, DU145, DuCaP, EL4, EM2, EM3, EMT6/AR1, EMT6/AR10.0, FM3, H1299, H69, HB54, HB55, HCA2, HEK-293, HeLa, Hepa1c1c7, HL-60, HMEC, HT-29, Jurkat, JY cells, K562 cells, Ku812, KCL22, KG1, KYO1, LNCap, Ma-Mel 1-48, MC-38, MCF-7, MCF-10A, MDA-MB-231, MDA-MB-468, MDA-MB-435, MDCK II, MDCK 11, MOR/0.2R, MONO-MAC 6, MTD-1A, MyEnd, NCI-H69/CPR, NCI-H69/LX10, NCI-H69/LX20, NCI-H69/LX4, NIH-3T3, NALM-1, NW-145, OPCN/OPCT cell lines, Peer, PNT-1A/PNT 2, RenCa, RIN-5F, RMA/RMAS, Saos-2 cells, Sf-9, SkBr3, T2, T-47D, T84, THP1 cell line, U373, U87, U937, VCaP, Vero cells, WM39, WT-49, X63, YAC-1, YAR, and transgenic varieties thereof.

Cell lines are available from a variety of sources known to those with skill in the art (see, e.g., the American Type Culture Collection (ATCC) (Manassas, Va.)). In some embodiments, a cell transfected with one or more vectors described herein is used to establish a new cell line comprising one or more vector-derived sequences. In some embodiments, a cell transiently transfected with the components of a CRISPR system as described herein (such as by transient transfection of one or more vectors, or transfection with RNA), and modified through the activity of a CRISPR complex, is used to establish a new cell line comprising cells containing the modification but lacking any other exogenous sequence. In some embodiments, cells transiently or non-transiently transfected with one or more vectors described herein, or cell lines derived from such cells, are used in assessing one or more test compounds.

### EXAMPLES

### Example 1. Creation of Improved ZF Scaffolds Optimized for Higher Efficiency ZF-DdCBEs

### Optimized Zif268-Derived ZF Scaffolds

Natural ZF arrays are found in transcription factors that localize to the nucleus inside mammalian cells. This occurs due the cryptic nuclear localization signals (NLSs) that are present in canonical ZF arrays. These NLS motifs are located within the DNA binding domains and impair the localization of ZF-DdCBEs to the mitochondria, limiting mitochondrial base editing activity. It is important to remove these NLS motifs without compromising the ability for ZFs to bind their target DNA sequences.

ZF arrays normally consist of between 3 and 6 individual ZF repeats. Each individual ZF repeat consists of (i) an alpha-helical motif, (ii) seven variable DNA-binding residues (which specify the target DNA sequence), and (iii) a beta-sheet motif. Individual ZF repeats are then joined together by a flexible linker motif. In both natural ZF arrays and designed ZF arrays, the sequences of the alpha-helical motif, beta-sheet motif, and a flexible linker motif all commonly vary between individual ZF repeats.

Work was performed to establish an optimized ZF sequence in which the alpha-helical motif, beta-sheet motif, and a flexible linker motif were identical for every ZF repeat within a ZF array. It was hypothesized that a particular combination of alpha-helical motif, beta-sheet motif, and flexible linker motif would be optimal for a ZF-DdCBE and would give rise to the highest on-target editing activity, compared to other combinations.

A computational tool, cNLS Mapper (nls-mapper.iab.keio.ac.jp/cgi-bin/NLS_Mapper_form.cgi) that scores the predicted NLS strength within a given protein sequence was used to test all possible different permutations of ZF arrays built and score these for predicted NLS strength.

For ZF arrays derived from the Zif268 sequence, it was found that the FQCRICMRNFS (SEQ ID NO: 396) alpha-helical motif was preferable to FACDICGRKFA (SEQ ID NO: 345); the HIRTH (SEQ ID NO: 346) beta-sheet motif was preferable to HTKIH (SEQ ID NO: 397); and the TGEKP (SEQ ID NO: 1) flexible linker motif was preferable to TGQKP (SEQ ID NO: 449). These gave rise to ZF arrays with a lower predicted NLS strength according to cNLS Mapper, and in combination gave the lowest possible precited NLS strength.

This particular combination (FQCRICMRNFS (SEQ ID NO: 396), HIRTH (SEQ ID NO: 346), TGEKP (SEQ ID NO: 1)) was designated as an “optimized” ZF scaffold, and it was demonstrated using two different ZF-DdCBE pairs that this gave higher editing efficiency compared to ZF-DdCBEs designed using the canonical ZF scaffold.

### Optimized Sp1C-Derived ZF Scaffolds

ZFs are most commonly designed using sequences derived from the natural Zif268 scaffold. An alternative natural scaffold from which to design ZFs is the Sp1C scaffold. The Zif268 and Sp1C scaffolds share the same beta-sheet motifs and flexible linker motifs but differ in their alpha-helical motif sequences. The Sp1C scaffold uses two different sequences for the alpha-helical motif of each ZF repeat within a ZF array—one of which is YKCPECGKSFS (SEQ ID NO: 336), and the other of which is YACPVESCDRRFS (SEQ ID NO: 342). As shown in the sequence alignment below (SEQ ID NOs: 336, 342), these naturally differ in two aspects:

Sp1C YKCP-E-CGKSFS
Sp1C YACPVESCDRRFS
* ** * * : **

Firstly, there is an insertion of two residues (V and S). Secondly, the identity of the amino acids at positions 2 and 7-9 in this motif are changed from K . . . GKS to A . . . DRR.

It was investigated whether alpha helical motifs derived from Sp1C conferred advantages over the Zif268alpha helical motif, in the context of an optimized ZF scaffold.

ZF arrays exclusively containing the shorter YKCPECGKSFS (SEQ ID NO: 336) Sp1C alpha-helical motif were created, and this scaffold was named K-GKS according to the identity of the amino acids at positions 2 and 7-9 in this motif. A set of different ZF arrays were then created in which the Sp1C alpha-helical motif was successively mutated at residues 2, 7, 8, and 9 to incrementally change these residues to the sequences found in the longer YACPVESCDRRFS (SEQ ID NO: 342) Sp1C motif. These were named A-GKS, A-GRS, A-DRS and A-DRR.

Next, ZF arrays exclusively containing the longer YACPVESCDRRFS (SEQ ID NO: 342) Sp1C alpha-helical motif were created, and this scaffold was named VS-DRR according to the identity of the amino acids at positions 5, 7 and 9-11 in this motif. A set of different ZF arrays were then created in which the Sp1C alpha-helical motif was successively mutated at residues 5, 7, 9, 10, and 11 to incrementally change these residues to the sequences found in the shorter YKCPECGKSFS (SEQ ID NO: 336) Sp1C motif. These were named VS-DRS, VS-GRS, and VS-GKS.

ZF-DdCBEs designed using these ZF scaffolds were tested to determine which gave the highest editing efficiency. Across the ZF-DdCBEs tested, it was found that the A-GKS alpha-helical motif derived from Sp1C, in combination with the earlier optimized ZF scaffold, gave rise to the highest editing efficiency.

Taken together, these results enabled the definition of a new ZF scaffold specifically optimized for mitochondrial localization, as evidenced by increased editing efficiency.

### Further Optimized Zinc Finger Scaffolds

Canonical ZF arrays derived from the Zif268 sequence can be constructed by using either FQCRICMRNFS (SEQ ID NO: 396) or FACDICGRKFA (SEQ ID NO: 345) as the alpha-helical motif sequence, HIRTH (SEQ ID NO: 346) or HTKIH (SEQ ID NO: 397) as the beta-sheet motif sequence, and TGEKP (SEQ ID NO: 1) or TGQKP (SEQ ID NO: 449) as the linker motif sequence. To determine the optimal combination of these sequences, all eight combinations of these sequences were constructed and tested. It was found that permutation X1 was consistently the best ZF scaffold architecture and gave rise to significantly higher base editing activity. In all permutations tested, the beta-sheet motif FACDICGRKFA (SEQ ID NO: 345) outperformed FQCRICMRNFS (SEQ ID NO: 396); the alpha-helical motif HIRTH (SEQ ID NO: 346) outperformed HTKIH (SEQ ID NO: 397); and the flexible linker motif TGEKP (SEQ ID NO: 1) outperformed TGQKP (SEQ ID NO: 449). The sequences in these three motifs appear to be able to be mixed and matched in an independent fashion, and thus are interchangeable.

These results were consistent when ZF-DdCBEs constructed from 5ZF arrays were tested at two different sites (site ATP8 and site ND5.1), and these results were also consistent when ZF-DdCBEs constructed from either 3ZF arrays or 5ZF arrays were tested at the same site (ATP8). Therefore, these findings seem to be generally applicable at different sites and with different ZF array lengths.

To explore whether there were other ZF scaffold sequences that could confer even higher base editing activity to ZF-DdCBEs than the canonical Zif268-derived sequences, the human proteome was searched for the ZF consensus sequence: x(2)-C-x(2,4)-C-x(12)-H-x(3)-H-x(4,5)-P, where C and H are conserved Cys and His residues that coordinate the Zn², ion, P is a conserved Pro residue at the end of the linker motif, and x can be any amino acid residue. This search query found a very large number of ZF sequences that are naturally occurring in the human proteome. These sequences were separated and filtered to extract new beta-motif sequences, new alpha-helical motif sequences, and new linker motif sequences. All the sequences identified were aligned within each class, and an amino acid frequency calculation was performed to determine the frequency at which each of the 20 amino acids were found at each position within the motif sequences. This analysis was performed with and without removing duplicate sequences after the query search, and the results were approximately consistent. A cut-off filter of 10% frequency was chosen, and amino acids that occurred at a frequency higher than 10% at each amino acid position were retained. This provided a basis set of amino acids from which to construct new motif sequences. All possible permutations of these sequences were tested, which resulted in the creation of 24 linker motifs, 12 alpha-motifs, and 96 beta-motifs. ZF-DdCBEs designed to edit site ATP8 were constructed based on the X1 architecture, in which either the linker motif only (YL series), the alpha-motif only (YA series), or the beta-motif only (YB series) was changed. The YL, YA and YB series were tested against the architecture to determine if any of these new ZF scaffold sequences could offer any further improvements.

It was found that top hits in the YL series displayed equivalent editing activity to the X1 architecture. However, it was found that top hits in each of the YA and YB series could outperform the X1 architecture.

A finalized ZF architecture was also constructed and tested that combined the best hits from the YA and YB series into the X1 architecture to see if these can combine additively and create an optimized ZF scaffold sequence that confers substantially improved base editing activity over the canonical Zif268-derived scaffold.

Several ZF scaffold sequences have been defined, including the “X1” scaffold (every beta-motif is FACDICGRKFA (SEQ ID NO: 345), every alpha-motif is HIRTH (SEQ ID NO: 346), and every linker motif is TGEKP (SEQ ID NO: 1)), the “AGKS” scaffold (every beta-motif is YACPECGKSFS (SEQ ID NO: 337), every alpha-motif is HIRTH (SEQ ID NO: 346), and every linker motif is TGEKP (SEQ ID NO: 1)), the “V10” scaffold (every beta-motif is FKCEECGKAFN (SEQ ID NO: 111), every alpha-motif is HIRTH (SEQ ID NO: 346), and every linker motif is TGEKP (SEQ ID NO: 1)), and the “V20” scaffold (every beta-motif is YKCEECGKAFN (SEQ ID NO: 63), every alpha-motif is HIRTH (SEQ ID NO: 346), and every linker motif is TGEKP (SEQ ID NO: 1)).

### Zinc Finger Linker Sequences:

(SEQ ID NO: 1)
TGEKP
(SEQ ID NO: 2)
TGERP
(SEQ ID NO: 3)
TGKKP
(SEQ ID NO: 4)
TGKRP
(SEQ ID NO: 5)
TGDKP
(SEQ ID NO: 6)
TGDRP
(SEQ ID NO: 7)
TEEKP
(SEQ ID NO: 8)
TEERP
(SEQ ID NO: 9)
TEKKP
(SEQ ID NO: 10)
TEKRP
(SEQ ID NO: 11)
TEDKP
(SEQ ID NO: 12)
TEDRP
(SEQ ID NO: 13)
SGEKP
(SEQ ID NO: 14)
SGERP
(SEQ ID NO: 15)
SGKKP
(SEQ ID NO: 16)
SGKRP
(SEQ ID NO: 17)
SGDKP
(SEQ ID NO: 18)
SGDRP
(SEQ ID NO: 19)
SEEKP
(SEQ ID NO: 20)
SEERP
(SEQ ID NO: 21)
SEKKP
(SEQ ID NO: 22)
SEKRP
(SEQ ID NO: 23)
SEDKP
(SEQ ID NO: 24)
SEDRP

### Zinc Finger α-Motif Sequences:

(SEQ ID NO: 25)
HQRIH
(SEQ ID NO: 26)
HQRVH
(SEQ ID NO: 27)
HQRTH
(SEQ ID NO: 28)
HQKIH
(SEQ ID NO: 29)
HQKVH
(SEQ ID NO: 30)
HQKTH
(SEQ ID NO: 31)
HMRIH
(SEQ ID NO: 32)
HMRVH
(SEQ ID NO: 33)
HMRTH
(SEQ ID NO: 34)
HMKIH
(SEQ ID NO: 35)
HMKVH
(SEQ ID NO: 36)
HMKTH
(SEQ ID NO: 37)
HKRIH
(SEQ ID NO: 38)
HKRVH
(SEQ ID NO: 39)
HKRTH
(SEQ ID NO: 40)
HKKIH
(SEQ ID NO: 41)
HKKVH
(SEQ ID NO: 42)
HKKTH
(SEQ ID NO: 346)
HIRTH

### Zinc Finger β-Motif Sequences:

(SEQ ID NO: 43)
YKCKECGKAFS
(SEQ ID NO: 44)
YKCKECGKAFR
(SEQ ID NO: 45)
YKCKECGKAFN
(SEQ ID NO: 46)
YKCKECGKSFS
(SEQ ID NO: 47)
YKCKECGKSFR
(SEQ ID NO: 48)
YKCKECGKSFN
(SEQ ID NO: 49)
YKCNECGKAFS
(SEQ ID NO: 50)
YKCNECGKAFR
(SEQ ID NO: 51)
YKCNECGKAFN
(SEQ ID NO: 52)
YKCNECGKSFS
(SEQ ID NO: 53)
YKCNECGKSFR
(SEQ ID NO: 54)
YKCNECGKSFN
(SEQ ID NO: 55)
YKCSECGKAFS
(SEQ ID NO: 56)
YKCSECGKAFR
(SEQ ID NO: 57)
YKCSECGKAFN
(SEQ ID NO: 58)
YKCSECGKSFS
(SEQ ID NO: 59)
YKCSECGKSFR
(SEQ ID NO: 60)
YKCSECGKSFN
(SEQ ID NO: 61)
YKCEECGKAFS
(SEQ ID NO: 62)
YKCEECGKAFR
(SEQ ID NO: 63)
YKCEECGKAFN
(SEQ ID NO: 64)
YKCEECGKSFS
(SEQ ID NO: 65)
YKCEECGKSFR
(SEQ ID NO: 66)
YKCEECGKSFN
(SEQ ID NO: 67)
YECKECGKAFS
(SEQ ID NO: 68)
YECKECGKAFR
(SEQ ID NO: 69)
YECKECGKAFN
(SEQ ID NO: 70)
YECKECGKSFS
(SEQ ID NO: 71)
YECKECGKSFR
(SEQ ID NO: 72)
YECKECGKSFN
(SEQ ID NO: 73)
YECNECGKAFS
(SEQ ID NO: 74)
YECNECGKAFR
(SEQ ID NO: 75)
YECNECGKAFN
(SEQ ID NO: 76)
YECNECGKSFS
(SEQ ID NO: 77)
YECNECGKSFR
(SEQ ID NO: 78)
YECNECGKSFN
(SEQ ID NO: 79)
YECSECGKAFS
(SEQ ID NO: 80)
YECSECGKAFR
(SEQ ID NO: 81)
YECSECGKAFN
(SEQ ID NO: 82)
YECSECGKSFS
(SEQ ID NO: 83)
YECSECGKSFR
(SEQ ID NO: 84)
YECSECGKSFN
(SEQ ID NO: 85)
YECEECGKAFS
(SEQ ID NO: 86)
YECEECGKAFR
(SEQ ID NO: 87)
YECEECGKAFN
(SEQ ID NO: 88)
YECEECGKSFS
(SEQ ID NO: 89)
YECEECGKSFR
(SEQ ID NO: 90)
YECEECGKSFN
(SEQ ID NO: 91)
FKCKECGKAFS
(SEQ ID NO: 92)
FKCKECGKAFR
(SEQ ID NO: 93)
FKCKECGKAFN
(SEQ ID NO: 94)
FKCKECGKSFS
(SEQ ID NO: 95)
FKCKECGKSFR
(SEQ ID NO: 96)
FKCKECGKSFN
(SEQ ID NO: 97)
FKCNECGKAFS
(SEQ ID NO: 98)
FKCNECGKAFR
(SEQ ID NO: 99)
FKCNECGKAFN
(SEQ ID NO: 100)
FKCNECGKSFS
(SEQ ID NO: 101)
FKCNECGKSFR
(SEQ ID NO: 102)
FKCNECGKSFN
(SEQ ID NO: 103)
FKCSECGKAFS
(SEQ ID NO: 104)
FKCSECGKAFR
(SEQ ID NO: 105)
FKCSECGKAFN
(SEQ ID NO: 106)
FKCSECGKSFS
(SEQ ID NO: 107)
FKCSECGKSFR
(SEQ ID NO: 108)
FKCSECGKSFN
(SEQ ID NO: 109)
FKCEECGKAFS
(SEQ ID NO: 110)
FKCEECGKAFR
(SEQ ID NO: 111)
FKCEECGKAFN
(SEQ ID NO: 112)
FKCEECGKSFS
(SEQ ID NO: 113)
FKCEECGKSFR
(SEQ ID NO: 114)
FKCEECGKSFN
(SEQ ID NO: 115)
FECKECGKAFS
(SEQ ID NO: 116)
FECKECGKAFR
(SEQ ID NO: 117)
FECKECGKAFN
(SEQ ID NO: 118)
FECKECGKSFS
(SEQ ID NO: 119)
FECKECGKSFR
(SEQ ID NO: 120)
FECKECGKSFN
(SEQ ID NO: 121)
FECNECGKAFS
(SEQ ID NO: 122)
FECNECGKAFR
(SEQ ID NO: 123)
FECNECGKAFN
(SEQ ID NO: 124)
FECNECGKSFS
(SEQ ID NO: 125)
FECNECGKSFR
(SEQ ID NO: 126)
FECNECGKSFN
(SEQ ID NO: 127)
FECSECGKAFS
(SEQ ID NO: 128)
FECSECGKAFR
(SEQ ID NO: 129)
FECSECGKAFN
(SEQ ID NO: 130)
FECSECGKSFS
(SEQ ID NO: 131)
FECSECGKSFR
(SEQ ID NO: 132)
FECSECGKSFN
(SEQ ID NO: 133)
FECEECGKAFS
(SEQ ID NO: 134)
FECEECGKAFR
(SEQ ID NO: 135)
FECEECGKAFN
(SEQ ID NO: 136)
FECEECGKSFS
(SEQ ID NO: 137)
FECEECGKSFR
(SEQ ID NO: 138)
FECEECGKSFN
(SEQ ID NO: 336)
YKCPECGKSFS
(SEQ ID NO: 337)
YACPECGKSFS
(SEQ ID NO: 338)
YACPECGRSFS
(SEQ ID NO: 339)
YACPECDRSFS
(SEQ ID NO: 340)
YACPECDRSFS
(SEQ ID NO: 341)
YACPECDRRFS
(SEQ ID NO: 342)
YACPVESCDRRFS
(SEQ ID NO: 343)
YACPVESCDRSFS
(SEQ ID NO: 344)
YACPVESCGKSFS
(SEQ ID NO: 345)
FACDICGRKFA

### Example 2. Creation of Specificity-Optimized ZF-DdCBEs with Lower Off-Target Editing Efficiency

An ideal DdCBE would exhibit high on-target editing efficiency, but low or no off-target editing. The spontaneous reassembly of split DddA halves can lead to off-target deamination independent from the on-target site, which, if not controlled, causes unwanted mutagenesis of the mitochondrial genome.

First, it was identified that treatment with ZF-DdCBEs leads to off-target editing in addition to the intended on-target editing. At the on-target site ATP8, there is targeted C-to-T conversion of 22%, which represents the desired on-target editing. However, within the same region of mtDNA, this is accompanied by the introduction of unwanted C-to-T or G-to-A edits of up to 3% when compared with the untreated control. This off-target editing was seen at two other sites in the mtDNA (ND5.1 and V1).

It was hypothesized that weakening the interaction affinity between the two DddA halves could fine-tune the deaminase activity to eliminate its off-target activity while still preserving high on-target editing efficiency.

### Truncation

It was hypothesized that truncation of the N-terminal DddA fragment (G1397N) and/or truncation of the C-terminal DddA fragment (G1397C) would reduce the interaction interface between the two split DddA halves and weaken the spontaneous reassembly of DddA off-target sites.

Truncations of the N-terminal DddA fragment (G1397N) at its C-terminus were created by deletion of between 1-10 amino acids. This was tested in combination with truncation of the C-terminal DddA fragment (G1397C) at its N-terminus by deletion of between 1-15 amino acids or truncation of the C-terminal DddA fragment (G1397C) at its C-terminus by deletion of between 1-15 amino acids.

It was found that off-target editing was reduced by truncation of the N-terminal DddA fragment (G1397N) at its C-terminus by deletion of 3 amino acids without any observed lowering on-target editing (Cd3). This produced an even greater effect when combined with truncation of the C-terminal DddA fragment (G1397C) at its N-terminus by deletion of 5 amino acids (Nd5).

### Point Mutations

It was hypothesized that introduction of individual point mutations in the C-terminal DddA fragment (G1397C) would reduce the interaction interface between the two split DddA halves and weaken the spontaneous reassembly of DddA off-target sites.

Alanine scanning (to remove side chain interactions), Lysine scanning (to introduce positive charge), and Glutamate and Aspartate scanning (to introduce negative charge) were tested. In this way, 120 constructs were tested in which each of the 30 residues in the C-terminal DddA fragment (G1397C) was individually mutated to either Ala, Lys, Glu or Asp. Point mutants that gave lower off-target editing without decreasing on-target editing, or point mutations that gave large reductions in off-target editing with only minor decreases in off-target editing, were observed, including: A5, A6, A7, A9, A14, A25, K12, K14, K18, K25, D3, D4, D5, D9, D14, D18, D19, D20, D25, D27, E5, E13, E16 and E20.

In particular, the four individual point mutations that gave the greatest reduction in off-target editing without decreasing on-target editing were D20, E20, K18, and K25.

### Charged Sequences Upstream

It was hypothesized that introduction of charged residues in the flexible linker between the ZF and the split DddA halves would introduce electrostatic repulsion that would weaken the spontaneous reassembly of DddA off-target sites.

ZF-DdCBE constructs were created in which the 13-amino acid flexible linker (GSGGGGSGGSGGS (SEQ ID NO: 309)) was mutated by introducing either 3, 6 or 9 consecutive negatively-charged residues (either Asp or Glu): GSGGGGSGDDDGS (SEQ ID NO: 319), GSGGGDDDDDDGS (SEQ ID NO: 320), GSDDDDDDDDDGS (SEQ ID NO: 321), GSGGGGSGGSDDD (SEQ ID NO: 316), GSGGGGSDDDDDD (SEQ ID NO: 317), GSGGDDDDDDDDD (SEQ ID NO: 318), GSGGGGSGEEEGS (SEQ ID NO: 313), GSGGGEEEEEEGS (SEQ ID NO: 314), GSEEEEEEEEEGS (SEQ ID NO: 315), GSGGGGSGGSEEE (SEQ ID NO: 310), GSGGGGSEEEEEE (SEQ ID NO: 311), and GSGGEEEEEEEEE (SEQ ID NO: 312).

Constructs were also tested in which the 4-amino acid flexible linker (SGGS) between the N-terminal DddA fragment (G1397N) and the UGI was replaced with linker sequences containing either 3, 6 or 9 consecutive negatively-charged residues (either Asp or Glu): SGDDDGS (SEQ ID NO: 236), SGDDDDDDGS (SEQ ID NO: 327), SGDDDDDDDDDGS (SEQ ID NO: 328), DDDGS (SEQ ID NO: 323), DDDDDDGS (SEQ ID NO: 324), DDDDDDDDDGS (SEQ ID NO: 325), SGDDDGS (SEQ ID NO: 236), SGDDDDDDGS (SEQ ID NO: 327), SGDDDDDDDDDGS (SEQ ID NO: 328), DDDGS (SEQ ID NO: 323), DDDDDDGS (SEQ ID NO: 324), and DDDDDDDDDGS (SEQ ID NO: 325).

Constructs that gave lower off-target editing without decreasing on-target editing, or point mutations that gave large reductions in off-target editing with only minor decreases in off-target editing, were observed.

Capping with a Catalytically-Inactivated (Dead) Deaminase

DddA can be catalytically inactivated by introduction of a E1347A mutation. In the G1397-split architecture, this mutation lies in the N-terminal DddA fragment (G1397N).

It was hypothesized that fusing a catalytically-inactivated N-terminal DddA fragment (G1397N) adjacent to the C-terminal DddA fragment (G1397C) would compete for reassembly and would weaken the spontaneous reassembly of catalytically-active DddA off-target sites.

ZF-DdCBE constructs were created in which a catalytically-inactivated N-terminal DddA fragment (G1397N) was fused downstream of the C-terminal DddA fragment (G1397C), either before or after the UGI, using flexible linkers of different lengths.

Constructs that gave lower off-target editing without decreasing on-target editing, or point mutations that gave large reductions in off-target editing with only minor decreases in off-target editing, were observed.

Overall, double-stranded DNA deaminase (DddA) mutants comprising point mutations, truncations, extensions, and dead deaminase caps were tested. Various combinations were also tested. Mutants comprising an N18K mutation, N18K and P25A mutations, and N18K and P25K mutations showed particularly promising increases in activity. Variants comprising a truncation of the three C-terminal amino acids of the N-terminal DddA fragment also showed particularly promising increases in activity, especially in combination with N18K and/or P25A or P25K mutations.

### Point Mutations in DddA C-Terminal Fragment G1397C:

| Mutation: | Sequence: |
|---|---|
| Canonical | AIPVKRGATGETKVFTGNSNSPKSPTKGGC (SEQ ID NO: 139) |
| I2A | AAPVKRGATGETKVFTGNSNSPKSPTKGGC (SEQ ID NO: 140) |
| P3A | AIAVKRGATGETKVFTGNSNSPKSPTKGGC (SEQ ID NO: 141) |
| V4A | AIPAKRGATGETKVFTGNSNSPKSPTKGGC (SEQ ID NO: 142) |
| K5A | AIPVARGATGETKVFTGNSNSPKSPTKGGC (SEQ ID NO: 143) |
| R6A | AIPVKAGATGETKVFTGNSNSPKSPTKGGC (SEQ ID NO: 144) |
| G7A | AIPVKRAATGETKVFTGNSNSPKSPTKGGC (SEQ ID NO: 145) |
| T9A | AIPVKRGAAGETKVFTGNSNSPKSPTKGGC (SEQ ID NO: 146) |
| G10A | AIPVKRGATAETKVFTGNSNSPKSPTKGGC (SEQ ID NO: 147) |
| E11A | AIPVKRGATGATKVFTGNSNSPKSPTKGGC (SEQ ID NO: 148) |
| T12A | AIPVKRGATGEAKVFTGNSNSPKSPTKGGC (SEQ ID NO: 149) |
| K13A | AIPVKRGATGETAVFTGNSNSPKSPTKGGC (SEQ ID NO: 150) |
| V14A | AIPVKRGATGETKAFTGNSNSPKSPTKGGC (SEQ ID NO: 151) |
| F15A | AIPVKRGATGETKVATGNSNSPKSPTKGGC (SEQ ID NO: 152) |
| T16A | AIPVKRGATGETKVFAGNSNSPKSPTKGGC (SEQ ID NO: 153) |
| G17A | AIPVKRGATGETKVFTANSNSPKSPTKGGC (SEQ ID NO: 154) |
| N18A | AIPVKRGATGETKVFTGASNSPKSPTKGGC (SEQ ID NO: 155) |
| S19A | AIPVKRGATGETKVFTGNANSPKSPTKGGC (SEQ ID NO: 156) |
| N20A | AIPVKRGATGETKVFTGNSASPKSPTKGGC (SEQ ID NO: 157) |
| S21A | AIPVKRGATGETKVFTGNSNAPKSPTKGGC (SEQ ID NO: 158) |
| P22A | AIPVKRGATGETKVFTGNSNSAKSPTKGGC (SEQ ID NO: 159) |
| K23A | AIPVKRGATGETKVFTGNSNSPASPTKGGC (SEQ ID NO: 160) |
| S24A | AIPVKRGATGETKVFTGNSNSPKAPTKGGC (SEQ ID NO: 161) |
| P25A | AIPVKRGATGETKVFTGNSNSPKSATKGGC (SEQ ID NO: 162) |
| T26A | AIPVKRGATGETKVFTGNSNSPKSPAKGGC (SEQ ID NO: 163) |
| K27A | AIPVKRGATGETKVFTGNSNSPKSPTAGGC (SEQ ID NO: 164) |
| G28A | AIPVKRGATGETKVFTGNSNSPKSPTKAGC (SEQ ID NO: 165) |
| G29A | AIPVKRGATGETKVFTGNSNSPKSPTKGAC (SEQ ID NO: 166) |
| C30A | AIPVKRGATGETKVFTGNSNSPKSPTKGGA (SEQ ID NO: 167) |
| A1K | KIPVKRGATGETKVFTGNSNSPKSPTKGGC (SEQ ID NO: 168) |
| I2K | AKPVKRGATGETKVFTGNSNSPKSPTKGGC (SEQ ID NO: 169) |
| P3K | AIKVKRGATGETKVFTGNSNSPKSPTKGGC (SEQ ID NO: 170) |
| V4K | AIPKKRGATGETKVFTGNSNSPKSPTKGGC (SEQ ID NO: 171) |
| R6K | AIPVKKGATGETKVFTGNSNSPKSPTKGGC (SEQ ID NO: 172) |
| G7K | AIPVKRKATGETKVFTGNSNSPKSPTKGGC (SEQ ID NO: 173) |
| A8K | AIPVKRGKTGETKVFTGNSNSPKSPTKGGC (SEQ ID NO: 174) |
| T9K | AIPVKRGAKGETKVFTGNSNSPKSPTKGGC (SEQ ID NO: 175) |
| G10K | AIPVKRGATKETKVFTGNSNSPKSPTKGGC (SEQ ID NO: 176) |
| E11K | AIPVKRGATGKTKVFTGNSNSPKSPTKGGC (SEQ ID NO: 177) |
| T12K | AIPVKRGATGEKKVFTGNSNSPKSPTKGGC (SEQ ID NO: 178) |
| V14K | AIPVKRGATGETKKFTGNSNSPKSPTKGGC (SEQ ID NO: 179) |
| F15K | AIPVKRGATGETKVKTGNSNSPKSPTKGGC (SEQ ID NO: 180) |
| T16K | AIPVKRGATGETKVFKGNSNSPKSPTKGGC (SEQ ID NO: 181) |
| G17K | AIPVKRGATGETKVFTKNSNSPKSPTKGGC (SEQ ID NO: 182) |
| N18K | AIPVKRGATGETKVFTGKSNSPKSPTKGGC (SEQ ID NO: 183) |
| S19K | AIPVKRGATGETKVFTGNKNSPKSPTKGGC (SEQ ID NO: 184) |
| N20K | AIPVKRGATGETKVFTGNSKSPKSPTKGGC (SEQ ID NO: 185) |
| S21K | AIPVKRGATGETKVFTGNSNKPKSPTKGGC (SEQ ID NO: 186) |
| P22K | AIPVKRGATGETKVFTGNSNSKKSPTKGGC (SEQ ID NO: 187) |
| S24K | AIPVKRGATGETKVFTGNSNSPKKPTKGGC (SEQ ID NO: 188) |
| P25K | AIPVKRGATGETKVFTGNSNSPKSKTKGGC (SEQ ID NO: 189) |
| T26K | AIPVKRGATGETKVFTGNSNSPKSPKKGGC (SEQ ID NO: 190) |
| G28K | AIPVKRGATGETKVFTGNSNSPKSPTKKGC (SEQ ID NO: 191) |
| G29K | AIPVKRGATGETKVFTGNSNSPKSPTKGKC (SEQ ID NO: 192) |
| C30K | AIPVKRGATGETKVFTGNSNSPKSPTKGGK (SEQ ID NO: 193) |
| A1D | DIPVKRGATGETKVFTGNSNSPKSPTKGGC (SEQ ID NO: 194) |
| I2D | ADPVKRGATGETKVFTGNSNSPKSPTKGGC (SEQ ID NO: 195) |
| P3D | AIDVKRGATGETKVFTGNSNSPKSPTKGGC (SEQ ID NO: 196) |
| V4D | AIPDKRGATGETKVFTGNSNSPKSPTKGGC (SEQ ID NO: 197) |
| K5D | AIPVDRGATGETKVFTGNSNSPKSPTKGGC (SEQ ID NO: 198) |
| R6D | AIPVKDGATGETKVFTGNSNSPKSPTKGGC (SEQ ID NO: 199) |
| G7D | AIPVKRDATGETKVFTGNSNSPKSPTKGGC (SEQ ID NO: 200) |
| A8D | AIPVKRGDTGETKVFTGNSNSPKSPTKGGC (SEQ ID NO: 201) |
| T9D | AIPVKRGADGETKVFTGNSNSPKSPTKGGC (SEQ ID NO: 202) |
| G10D | AIPVKRGATDETKVFTGNSNSPKSPTKGGC (SEQ ID NO: 203) |
| E11D | AIPVKRGATGDTKVFTGNSNSPKSPTKGGC (SEQ ID NO: 204) |
| T12D | AIPVKRGATGEDKVFTGNSNSPKSPTKGGC (SEQ ID NO: 205) |
| K13D | AIPVKRGATGETDVFTGNSNSPKSPTKGGC (SEQ ID NO: 206) |
| V14D | AIPVKRGATGETKDFTGNSNSPKSPTKGGC (SEQ ID NO: 207) |
| F15D | AIPVKRGATGETKVDTGNSNSPKSPTKGGC (SEQ ID NO: 208) |
| T16D | AIPVKRGATGETKVFDGNSNSPKSPTKGGC (SEQ ID NO: 209) |
| G17D | AIPVKRGATGETKVFTDNSNSPKSPTKGGC (SEQ ID NO: 210) |
| N18D | AIPVKRGATGETKVFTGDSNSPKSPTKGGC (SEQ ID NO: 211) |
| S19D | AIPVKRGATGETKVFTGNDNSPKSPTKGGC (SEQ ID NO: 212) |
| N20D | AIPVKRGATGETKVFTGNSDSPKSPTKGGC (SEQ ID NO: 213) |
| S21D | AIPVKRGATGETKVFTGNSNDPKSPTKGGC (SEQ ID NO: 214) |
| P22D | AIPVKRGATGETKVFTGNSNSDKSPTKGGC (SEQ ID NO: 215) |
| K23D | AIPVKRGATGETKVFTGNSNSPDSPTKGGC (SEQ ID NO: 216) |
| S24D | AIPVKRGATGETKVFTGNSNSPKDPTKGGC (SEQ ID NO: 217) |
| P25D | AIPVKRGATGETKVFTGNSNSPKSDTKGGC (SEQ ID NO: 218) |
| T26D | AIPVKRGATGETKVFTGNSNSPKSPDKGGC (SEQ ID NO: 219) |
| K27D | AIPVKRGATGETKVFTGNSNSPKSPTDGGC (SEQ ID NO: 220) |
| G28D | AIPVKRGATGETKVFTGNSNSPKSPTKDGC (SEQ ID NO: 221) |
| G29D | AIPVKRGATGETKVFTGNSNSPKSPTKGDC (SEQ ID NO: 222) |
| C30D | AIPVKRGATGETKVFTGNSNSPKSPTKGGD (SEQ ID NO: 223) |
| A1E | EIPVKRGATGETKVFTGNSNSPKSPTKGGC (SEQ ID NO: 224) |
| I2E | AEPVKRGATGETKVFTGNSNSPKSPTKGGC (SEQ ID NO: 225) |
| P3E | AIEVKRGATGETKVFTGNSNSPKSPTKGGC (SEQ ID NO: 226) |
| V4E | AIPEKRGATGETKVFTGNSNSPKSPTKGGC (SEQ ID NO: 227) |
| K5E | AIPVERGATGETKVFTGNSNSPKSPTKGGC (SEQ ID NO: 228) |
| R6E | AIPVKEGATGETKVFTGNSNSPKSPTKGGC (SEQ ID NO: 229) |
| G7E | AIPVKREATGETKVFTGNSNSPKSPTKGGC (SEQ ID NO: 230) |
| A8E | AIPVKRGETGETKVFTGNSNSPKSPTKGGC (SEQ ID NO: 231) |
| T9E | AIPVKRGAEGETKVFTGNSNSPKSPTKGGC (SEQ ID NO: 232) |
| G10E | AIPVKRGATEETKVFTGNSNSPKSPTKGGC (SEQ ID NO: 233) |
| T12E | AIPVKRGATGEEKVFTGNSNSPKSPTKGGC (SEQ ID NO: 234) |
| K13E | AIPVKRGATGETEVFTGNSNSPKSPTKGGC (SEQ ID NO: 235) |
| V14E | AIPVKRGATGETKEFTGNSNSPKSPTKGGC (SEQ ID NO: 236) |
| F15E | AIPVKRGATGETKVETGNSNSPKSPTKGGC (SEQ ID NO: 237) |
| T16E | AIPVKRGATGETKVFEGNSNSPKSPTKGGC (SEQ ID NO: 238) |
| G17E | AIPVKRGATGETKVFTENSNSPKSPTKGGC (SEQ ID NO: 239) |
| N18E | AIPVKRGATGETKVFTGESNSPKSPTKGGC (SEQ ID NO: 240) |
| S19E | AIPVKRGATGETKVFTGNENSPKSPTKGGC (SEQ ID NO: 241) |
| N20E | AIPVKRGATGETKVFTGNSESPKSPTKGGC (SEQ ID NO: 242) |
| S21E | AIPVKRGATGETKVFTGNSNEPKSPTKGGC (SEQ ID NO: 243) |
| P22E | AIPVKRGATGETKVFTGNSNSEKSPTKGGC (SEQ ID NO: 244) |
| K23E | AIPVKRGATGETKVFTGNSNSPESPTKGGC (SEQ ID NO: 245) |
| S24E | AIPVKRGATGETKVFTGNSNSPKEPTKGGC (SEQ ID NO: 246) |
| P25E | AIPVKRGATGETKVFTGNSNSPKSETKGGC (SEQ ID NO: 247) |
| T26E | AIPVKRGATGETKVFTGNSNSPKSPEKGGC (SEQ ID NO: 248) |
| K27E | AIPVKRGATGETKVFTGNSNSPKSPTEGGC (SEQ ID NO: 249) |
| G28E | AIPVKRGATGETKVFTGNSNSPKSPTKEGC (SEQ ID NO: 250) |
| G29E | AIPVKRGATGETKVFTGNSNSPKSPTKGEC (SEQ ID NO: 251) |
| C30E | AIPVKRGATGETKVFTGNSNSPKSPTKGGE (SEQ ID NO: 252) |

### N-Terminal Truncations of G1397C DddA Fragment:

| Truncation: | Sequence: |
|---|---|
| Canonical | AIPVKRGATGETKVFTGNSNSPKSPTKGGC (SEQ ID NO: 139) |
| NΔ1 | _IPVKRGATGETKVFTGNSNSPKSPTKGGC (SEQ ID NO: 253) |
| NΔ2 | __PVKRGATGETKVFTGNSNSPKSPTKGGC (SEQ ID NO: 254) |
| NΔ3 | ___VKRGATGETKVFTGNSNSPKSPTKGGC (SEQ ID NO: 255) |
| NΔ4 | ____KRGATGETKVFTGNSNSPKSPTKGGC (SEQ ID NO: 256) |
| NΔ5 | _____RGATGETKVFTGNSNSPKSPTKGGC (SEQ ID NO: 257) |
| NΔ6 | ______GATGETKVFTGNSNSPKSPTKGGC (SEQ ID NO: 258) |
| NΔ7 | _______ATGETKVFTGNSNSPKSPTKGGC (SEQ ID NO: 259) |
| NΔ8 | ________TGETKVFTGNSNSPKSPTKGGC (SEQ ID NO: 260) |
| NΔ9 | _________GETKVFTGNSNSPKSPTKGGC (SEQ ID NO: 261) |
| NΔ10 | __________ETKVFTGNSNSPKSPTKGGC (SEQ ID NO: 262) |
| NΔ11 | ___________TKVFTGNSNSPKSPTKGGC (SEQ ID NO: 263) |
| NΔ12 | ____________KVFTGNSNSPKSPTKGGC (SEQ ID NO: 264) |
| NΔ13 | _____________VFTGNSNSPKSPTKGGC (SEQ ID NO: 265) |
| NΔ14 | ______________FTGNSNSPKSPTKGGC (SEQ ID NO: 266) |
| NΔ15 | _______________TGNSNSPKSPTKGGC (SEQ ID NO: 267) |

### C-Terminal Truncations of G1397C DddA Fragment:

| Truncation: | Sequence: |
|---|---|
| Canonical | AIPVKRGATGETKVFTGNSNSPKSPTKGGC (SEQ ID NO: 139) |
| CΔ1 | AIPVKRGATGETKVFTGNSNSPKSPTKGG_ (SEQ ID NO: 268) |
| CΔ2 | AIPVKRGATGETKVFTGNSNSPKSPTKG__ (SEQ ID NO: 269) |
| CΔ3 | AIPVKRGATGETKVFTGNSNSPKSPTK___ (SEQ ID NO: 270) |
| CΔ4 | AIPVKRGATGETKVFTGNSNSPKSPT____ (SEQ ID NO: 271) |
| CΔ5 | AIPVKRGATGETKVFTGNSNSPKSP_____ (SEQ ID NO: 272) |
| CΔ6 | AIPVKRGATGETKVFTGNSNSPKS______ (SEQ ID NO: 273) |
| CΔ7 | AIPVKRGATGETKVFTGNSNSPK_______ (SEQ ID NO: 274) |
| CΔ8 | AIPVKRGATGETKVFTGNSNSP________ (SEQ ID NO: 275) |
| CΔ9 | AIPVKRGATGETKVFTGNSNS_________ (SEQ ID NO: 276) |
| CΔ10 | AIPVKRGATGETKVFTGNSN__________ (SEQ ID NO: 277) |
| CΔ11 | AIPVKRGATGETKVFTGNS___________ (SEQ ID NO: 278) |
| CΔ12 | AIPVKRGATGETKVFTGN____________ (SEQ ID NO: 279) |
| CΔ13 | AIPVKRGATGETKVFTG_____________ (SEQ ID NO: 280) |
| CΔ14 | AIPVKRGATGETKVFT______________ (SEQ ID NO: 281) |
| CΔ15 | AIPVKRGATGETKVF_______________ (SEQ ID NO: 282) |

### C-Terminal Truncations of G1397N Fragment:

| Truncation: | Sequence: |
|---|---|
| Canonical | GSYALGPYQISAPQLPAYNGQTVGTFYYVNDAGGLESKVFSSGGPTPYPNYAN |
| | AGHVEGQSALFMRDNGISEGLVFHNNPEGTCGFCVNMTETLLPENAKMTVVPP |
| | EG (SEQ ID NO: 283) |
| CΔ1 | GSYALGPYQISAPQLPAYNGQTVGTFYYVNDAGGLESKVFSSGGPTPYPNYAN |
| | AGHVEGQSALFMRDNGISEGLVFHNNPEGTCGFCVNMTETLLPENAKMTVVPP |
| | E_ (SEQ ID NO: 284) |
| CΔ2 | GSYALGPYQISAPQLPAYNGQTVGTFYYVNDAGGLESKVFSSGGPTPYPNYAN |
| | AGHVEGQSALFMRDNGISEGLVFHNNPEGTCGFCVNMTETLLPENAKMTVVPP |
| | __ (SEQ ID NO: 285) |
| CΔ3 | GSYALGPYQISAPQLPAYNGQTVGTFYYVNDAGGLESKVFSSGGPTPYPNYAN |
| | AGHVEGQSALFMRDNGISEGLVFHNNPEGTCGFCVNMTETLLPENAKMTVVP_ |
| | __ (SEQ ID NO: 286) |
| CΔ4 | GSYALGPYQISAPQLPAYNGQTVGTFYYVNDAGGLESKVFSSGGPTPYPNYAN |
| | AGHVEGQSALFMRDNGISEGLVFHNNPEGTCGFCVNMTETLLPENAKMTVV__ |
| | __ (SEQ ID NO: 287) |
| CΔ5 | GSYALGPYQISAPQLPAYNGQTVGTFYYVNDAGGLESKVFSSGGPTPYPNYAN |
| | AGHVEGQSALFMRDNGISEGLVFHNNPEGTCGFCVNMTETLLPENAKMTV___ |
| | __ (SEQ ID NO: 288) |
| CΔ6 | GSYALGPYQISAPQLPAYNGQTVGTFYYVNDAGGLESKVFSSGGPTPYPNYAN |
| | AGHVEGQSALFMRDNGISEGLVFHNNPEGTCGFCVNMTETLLPENAKMT____ |
| | __ (SEQ ID NO: 289) |
| CΔ7 | GSYALGPYQISAPQLPAYNGQTVGTFYYVNDAGGLESKVFSSGGPTPYPNYAN |
| | AGHVEGQSALFMRDNGISEGLVFHNNPEGTCGFCVNMTETLLPENAKM_____ |
| | __ (SEQ ID NO: 290) |
| CΔ8 | GSYALGPYQISAPQLPAYNGQTVGTFYYVNDAGGLESKVFSSGGPTPYPNYAN |
| | AGHVEGQSALFMRDNGISEGLVFHNNPEGTCGFCVNMTETLLPENAK______ |
| | __ (SEQ ID NO: 291) |
| CΔ9 | GSYALGPYQISAPQLPAYNGQTVGTFYYVNDAGGLESKVFSSGGPTPYPNYAN |
| | AGHVEGQSALFMRDNGISEGLVFHNNPEGTCGFCVNMTETLLPENA________ |
| | __ (SEQ ID NO: 292) |
| CΔ10 | GSYALGPYQISAPQLPAYNGQTVGTFYYVNDAGGLESKVFSSGGPTPYPNYAN |
| | AGHVEGQSALFMRDNGISEGLVFHNNPEGTCGFCVNMTETLLPEN_________ |
| | __ (SEQ ID NO: 293) |

### C-Terminal Extensions of G1397N Fragment:

| Extension: | Sequence: |
|---|---|
| Canonical | GSYALGPYQISAPQLPAYNGQTVGTFYYVNDAGGLESKVFSSGGPTPYPNYA |
| | NAGHVEGQSALFMRDNGISEGLVFHNNPEGTCGFCVNMTETLLPENAKMTVV |
| | PPEG (SEQ ID NO: 283) |
| C+1 | GSYALGPYQISAPQLPAYNGQTVGTFYYVNDAGGLESKVFSSGGPTPYPNYA |
| | NAGHVEGQSALFMRDNGISEGLVFHNNPEGTCGFCVNMTETLLPENAKMTVV |
| | PPEGA (SEQ ID NO: 294) |
| C+2 | GSYALGPYQISAPQLPAYNGQTVGTFYYVNDAGGLESKVFSSGGPTPYPNYA |
| | NAGHVEGQSALFMRDNGISEGLVFHNNPEGTCGFCVNMTETLLPENAKMTVV |
| | PPEGAI (SEQ ID NO: 295) |
| C+3 | GSYALGPYQISAPQLPAYNGQTVGTFYYVNDAGGLESKVFSSGGPTPYPNYA |
| | NAGHVEGQSALFMRDNGISEGLVFHNNPEGTCGFCVNMTETLLPENAKMTVV |
| | PPEGAIP (SEQ ID NO: 296) |
| C+4 | GSYALGPYQISAPQLPAYNGQTVGTFYYVNDAGGLESKVFSSGGPTPYPNYA |
| | NAGHVEGQSALFMRDNGISEGLVFHNNPEGTCGFCVNMTETLLPENAKMTVV |
| | PPEGAIPV (SEQ ID NO: 297) |
| C+5 | GSYALGPYQISAPQLPAYNGQTVGTFYYVNDAGGLESKVFSSGGPTPYPNYA |
| | NAGHVEGQSALFMRDNGISEGLVFHNNPEGTCGFCVNMTETLLPENAKMTVV |
| | PPEGAIPVK (SEQ ID NO: 298) |
| C+6 | GSYALGPYQISAPQLPAYNGQTVGTFYYVNDAGGLESKVFSSGGPTPYPNYA |
| | NAGHVEGQSALFMRDNGISEGLVFHNNPEGTCGFCVNMTETLLPENAKMTVV |
| | PPEGAIPVKR (SEQ ID NO: 299) |
| C+7 | GSYALGPYQISAPQLPAYNGQTVGTFYYVNDAGGLESKVFSSGGPTPYPNYA |
| | NAGHVEGQSALFMRDNGISEGLVFHNNPEGTCGFCVNMTETLLPENAKMTVV |
| | PPEGAIPVKRG (SEQ ID NO: 300) |
| C+8 | GSYALGPYQISAPQLPAYNGQTVGTFYYVNDAGGLESKVFSSGGPTPYPNYA |
| | NAGHVEGQSALFMRDNGISEGLVFHNNPEGTCGFCVNMTETLLPENAKMTVV |
| | PPEGAIPVKRGA (SEQ ID NO: 301) |
| C+9 | GSYALGPYQISAPQLPAYNGQTVGTFYYVNDAGGLESKVFSSGGPTPYPNYA |
| | NAGHVEGQSALFMRDNGISEGLVFHNNPEGTCGFCVNMTETLLPENAKMTVV |
| | PPEGAIPVKRGAT (SEQ ID NO: 302) |
| C+10 | GSYALGPYQISAPQLPAYNGQTVGTFYYVNDAGGLESKVFSSGGPTPYPNYA |
| | NAGHVEGQSALFMRDNGISEGLVFHNNPEGTCGFCVNMTETLLPENAKMTVV |
| | PPEGAIPVKRGATG (SEQ ID NO: 303) |
| C+11 | GSYALGPYQISAPQLPAYNGQTVGTFYYVNDAGGLESKVFSSGGPTPYPNYA |
| | NAGHVEGQSALFMRDNGISEGLVFHNNPEGTCGFCVNMTETLLPENAKMTVV |
| | PPEGAIPVKRGATGE (SEQ ID NO: 304) |
| C+12 | GSYALGPYQISAPQLPAYNGQTVGTFYYVNDAGGLESKVFSSGGPTPYPNYA |
| | NAGHVEGQSALFMRDNGISEGLVFHNNPEGTCGFCVNMTETLLPENAKMTVV |
| | PPEGAIPVKRGATGET (SEQ ID NO: 305) |
| C+13 | GSYALGPYQISAPQLPAYNGQTVGTFYYVNDAGGLESKVFSSGGPTPYPNYA |
| | NAGHVEGQSALFMRDNGISEGLVFHNNPEGTCGFCVNMTETLLPENAKMTVV |
| | PPEGAIPVKRGATGETK (SEQ ID NO: 306) |
| C+14 | GSYALGPYQISAPQLPAYNGQTVGTFYYVNDAGGLESKVFSSGGPTPYPNYA |
| | NAGHVEGQSALFMRDNGISEGLVFHNNPEGTCGFCVNMTETLLPENAKMTVV |
| | PPEGAIPVKRGATGETKV (SEQ ID NO: 307) |
| C+15 | GSYALGPYQISAPQLPAYNGQTVGTFYYVNDAGGLESKVFSSGGPTPYPNYA |
| | NAGHVEGQSALFMRDNGISEGLVFHNNPEGTCGFCVNMTETLLPENAKMTVV |
| | PPEGAIPVKRGATGETKVE (SEQ ID NO: 308) |

### Charged Residues Upstream or Downstream of Split DddA to Weaken Binding Affinity Between Split Halves and Lower Off-Target Activity:

(SEQ ID NO: 309)
GSGGGGSGGSGGS
(SEQ ID NO: 310)
GSGGGGSGGSEEE
(SEQ ID NO: 311)
GSGGGGSEEEEEE
(SEQ ID NO: 312)
GSGGEEEEEEEEE
(SEQ ID NO: 313)
GSGGGGSGEEEGS
(SEQ ID NO: 314)
GSGGGEEEEEEGS
(SEQ ID NO: 315)
GSEEEEEEEEEGS
(SEQ ID NO: 316)
GSGGGGSGGSDDD
(SEQ ID NO: 317)
GSGGGGSDDDDDD
(SEQ ID NO: 318)
GSGGDDDDDDDDD
(SEQ ID NO: 319)
GSGGGGSGDDDGS
(SEQ ID NO: 320)
GSGGGDDDDDDGS
(SEQ ID NO: 321)
GSDDDDDDDDDGS
(SEQ ID NO: 322)
SGGS
(SEQ ID NO: 323)
DDDGS
(SEQ ID NO: 324)
DDDDDDGS
(SEQ ID NO: 325)
DDDDDDDDDGS
(SEQ ID NO: 326)
SGDDDGS
(SEQ ID NO: 327)
SGDDDDDDGS
(SEQ ID NO: 328)
SGDDDDDDDDDGS
(SEQ ID NO: 329)
EEEGS
(SEQ ID NO: 330)
EEEEEEGS
(SEQ ID NO: 331)
EEEEEEEEEGS
(SEQ ID NO: 332)
SGEEEGS
(SEQ ID NO: 333)
SGEEEEEEGS
(SEQ ID NO: 334)
SGEEEEEEEEEGS

### Fusion of “Dead” DddA N-Terminal Domain to C-Terminal DddA Fragment to Reduce Off-Target Activity:

Canonical
(SEQ ID NO: 283)
GSYALGPYQISAPQLPAYNGQTVGTFYYVNDAGGLESKVFSSGGPTPYPN
YANAGHVEGQSALFMRDNGISEGLVFHNNPEGTCGFCVNMTETLLPENAK
MTVVPPEG
Dead (E1347A)
(SEQ ID NO: 335)
GSYALGPYQISAPQLPAYNGQTVGTFYYVNDAGGLESKVFSSGGPTPYPN
YANAGHVAGQSALFMRDNGISEGLVFHNNPEGTCGFCVNMTETLLPENAK
MTVVPPEG

ZF-DdCBE sequence
MTS
(SEQ ID NO: 402)
MLGFVGRVAAAPASGALRRLTPSASLPPAQLLLRAAPTAVHPVRDYAAQ
FLAG tag
(SEQ ID NO: 399)
DYKDDDDK
NES
(SEQ ID NO: 403)
VDEMTKKFGTLTIHDTEK
Linker
(SEQ ID NO: 400)
GS
NES2
(SEQ ID NO: 401)
LQKKLEELELD
Linker
(SEQ ID NO: 398)
AA
ZF
See below
Linker
(SEQ ID NO: 309)
GSGGGGSGGSGGS
Split DddA (DddA-G1397N or DddA-G1397C)
(SEQ ID NO: 283)
GSYALGPYQISAPQLPAYNGQTVGTFYYVNDAGGLESKVFSSGGPTPYPN
YANAGHVEGQSALFMRDNGISEGLVFHNNPEGTCGFCVNMTETLLPENAK
MTVVPPEG
or
(SEQ ID NO: 139)
AIPVKRGATGETKVFTGNSNSPKSPTKGGC
Linker
(SEQ ID NO: 322)
SGGS
UGI
(SEQ ID NO: 358)
TNLSDIIEKETGKQLVIQESILMLPEEVEEVIGNKPESDILVHTAYDEST
DENVMLLTSDAPEYKPWALVIQDSNGENKIKML
ZF sequences
R8
(SEQ ID NO: 404)
MAERPFQCRICMRNFSTSGSLSR
HIRTHTGEKPFACDICGRKFAQSGSLTRHTKIHTGGQRPFQCRICMRNFS
RSDALSQHIRTHTGEKPFACDICGRKFARNDNRITHTKIHTGEKPFQCRI
CMRKFARSDHLTQHTKIHLR
5xZnF-4-R8
(SEQ ID NO: 405)
MAERPFQCRICMRNFSQASNLISHIRTHTGEKPFACDICGRKFATSHSLT
EHTKIHTGSQKPFQCRICMRNFSERSHLREHIRTHTGEKPFACDICGRKF
AQSGNLTEHTKIHTGEKPFQCRICMRKFASKKALTEHTKIHLR
5xZnF-10-R8
(SEQ ID NO: 406)
MAERPFQCRICMRNFSQASNLISHIRTHTGEKPFACDICGRKFAQRANLR
AHTKIHTGSQKPFQCRICMRNFSQASNLISHIRTHTGEKPFACDICGRKF
ATSHSLTEHTKIHTGEKPFQCRICMRKFAERSHLREHTKIHLR
[403] R8-3i
(SEQ ID NO: 407)
MAERPFQCRICMRNFSTSGSLSRHIRTHTGEKPFACDICGRKFAQSGSLT
RHTKIHTGQKPFQCRICMRNFSRSDALSQHTKIHLR
3xZnF-4-R8_3i
(SEQ ID NO: 408)
MAERPFQCRICMRNFSQASNLISHIRTHTGEKPFACDICGRKFATSHSLT
EHTKIHTGQKPFQCRICMRNFSERSHLREHTKIHLR
3xZnF-10-R8_3ii
(SEQ ID NO: 409)
MAERPFQCRICMRNFSQRANLRAHIRTHTGEKPFACDICGRKFAQASNLI
SHTKIHTGQKPFQCRICMRNFSTSHSLTEHTKIHL
R13-1
(SEQ ID NO: 410)
MAERPFQCRICMRNFSRSDNLSTHIRTHTGEKPFACDICGRKFADRSDLS
RHTKIHTGEKPFQCRICMRKFAQSGDLTRHTKIHTGSQKPFQCRICMRNF
SRSDSLSAHIRTHTGEKPFACDICGRKFAQKATRITHTKIHLR
5xZnF-9-R13
(SEQ ID NO: 411)
MAERPFQCRICMRNFSQSSSLVRHIRTHTGEKPFACDICGRKFARSDNLV
RHTKIHTGSQKPFQCRICMRNFSQAGHLASHIRTHTGEKPFACDICGRKF
ARKDNLKNHTKIHTGEKPFQCRICMRKFARKDALRGHTKIHLR
5xZnF-12-R13
(SEQ ID NO: 412)
MAERPFQCRICMRNFSRSDHLTTHIRTHTGEKPFACDICGRKFAQSSSLV
RHTKIHTGSQKPFQCRICMRNFSRSDNLVRHIRTHTGEKPFACDICGRKF
AQAGHLASHTKIHTGEKPFQCRICMRKFARKDNLKNHTKIHLR

### Example 3. High-Performance, Compact Zinc Finger Base Editors that Precisely Edit Mitochondrial or Nuclear DNA In Vitro and In Vivo

DddA-derived cytosine base editors (DdCBEs) use programmable DNA-binding TALE repeat arrays, rather than CRISPR proteins, together with a split double-stranded DNA-specific cytidine deaminase (DddA) and a uracil glycosylase inhibitor (UGI) to mediate targeted C•G-to-T•A editing in nuclear, mitochondrial, and chloroplast DNA¹³. Zinc finger (ZF) arrays are programmable DNA-binding proteins that offer much smaller size, lower immunogenicity, and different targeting features compared to TALE arrays⁴. The development of zinc finger DdCBEs (ZF-DdCBEs) is described herein, as is the extensive improvement of their on-target editing performance through engineering their architectures, defining improved ZF scaffolds, and installing DddA activity-enhancing mutations. These resulting optimized ZF-DdCBEs yielded substantially higher mitochondrial editing efficiencies (averaging >3.6-fold higher over 17 tested target sites) than recently reported ZF deaminases (ZFDs). Four strategies were identified to minimize off-target editing by ZF-DdCBEs, and these approaches were integrated to engineer high-specificity variants with minimal off-target editing and efficient on-target editing. These optimized ZF-DdCBEs were used to install or correct disease-associated mutations in mitochondria and in the nucleus. Leveraging their small size, a single AAV9 was used to deliver in vivo optimized ZF-DdCBEs programmed to install m.7743G>A or m.3177G>A, mutations that cause mitochondrial myopathy or Leber's hereditary optic neuropathy, respectively, into post-natal mice, achieving average bulk quadriceps mitochondrial base editing efficiencies of 60% and 46%, respectively. These findings demonstrate a compact, all-protein in vitro and in vivo base editing platform for the precise editing of organelle or nuclear DNA without double-strand DNA breaks.

Mitochondria are essential organelles in almost all eukaryotic cells. Each mitochondrion among hundreds per cell contains tens of circular copies of mtDNA encoding a set of proteins, rRNAs, and tRNAs that facilitate mitochondrial ATP productions⁵⁻⁸. Mutations in the mitochondrial genome can give rise to mitochondrial genetic diseases such as mitochondrial encephalopathy, lactic acidosis, stroke-like episodes (MELAS), and Leber hereditary optic neuropathy (LHON), among many others⁹⁻¹². The ability to install precise sequence changes within mtDNA could be invaluable to study and potentially treat mitochondrial genetic diseases, which collectively afflict approximately one in 5,000 people¹³.

Base editors use programmable DNA-binding proteins together with a natural or laboratory-evolved DNA deaminase to mediate precise targeted sequence changes in DNA within human cells^{14, 15}. Because no system for the efficient import of nucleic acids into mitochondria has been identified thus far, CRISPR base editors, which require a guide RNA component, currently cannot be used effectively in mitochondria^{16, 17}.

In contrast, protein import into mitochondria is well-characterized¹⁸, raising the possibility that all-protein, CRISPR-free base editors might enable the precision editing of organellar as well as nuclear genomes. The discovery of the first dsDNA-specific cytidine deaminase (DddA) enabled the development of efficient CRISPR-free base editors that edit nuclear and organelle DNA¹. The first all-protein base editors, DdCBEs, use programmable DNA-binding TALE repeat array proteins together with a split DddA and a uracil glycosylase inhibitor (UGI) to mediate targeted C•G-to-T•A editing in nuclear, mitochondrial, and chloroplast DNA¹⁻³. Full-length DddA can be split at position G1397 into two catalytically inactive halves, a 108-residue N-terminal fragment (DddA^{N}) and a 30-amino acid C-terminal fragment (DddA^{C}). The binding of two TALE-split-DddA-UGI fusions to adjacent sites promotes the reassembly of functional DddA for deamination of target cytosines within the dsDNA spacing region between the adjacent target sites.

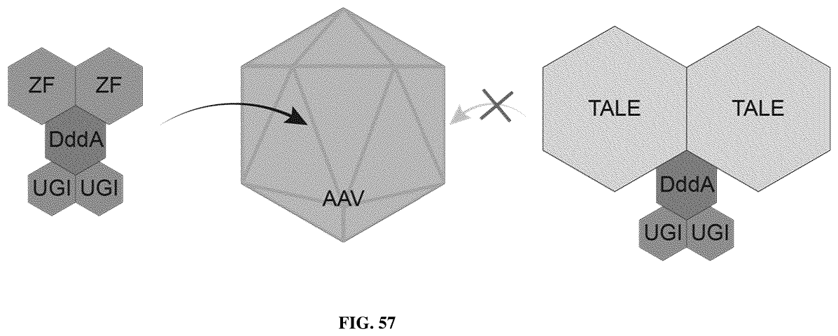

Due primarily to the large size of TALE repeat arrays, DdCBEs are too large to package in a single AAV construct for in vivo delivery, complicating their application in animals and as potential therapeutics (FIG. 57). TALE arrays can also be challenging to construct due to their repetitive sequence^{4, 19}, have certain target sequence requirements²⁰, and add a large number of immunogenic epitopes when fused to a protein. The development of all-protein zinc finger DdCBEs (ZF-DdCBEs) that can edit mitochondrial or nuclear DNA in vitro and in vivo is described herein. ZFs offer compact DNA recognition; each 28-residue ZF repeat recognizes three target nucleotides, while each 34-residue TALE repeat recognizes only a single nucleotide. In addition to being natively less repetitive in sequence and thus easier to construct, ZFs represent the most abundant class of proteins in the human proteome and are thought to be less immunogenic than most foreign proteins^{21, 22}. The development of ZF-DdCBEs thus offers more compact base editors with different targeting properties and potentially lower immunogenicity than TALE-based DdCBEs.

Efforts to develop ZF-targeted deaminases using a ZF array fused to activation-induced cytidine deaminase (AID)²³ have been previously reported. These efforts led to very low editing efficiencies in human cells because ZF arrays bind dsDNA, but all cytidine deaminases reported until 2020 require a ssDNA substrate²⁴. Independently, ZF deaminases (ZFDs) composed of a ZF array fused to split DddA and UGI were also reported²⁵. ZFDs support base editing of mitochondrial or nuclear DNA in vitro, but their optimization was primarily limited to the length of the amino acid linkers connecting the ZF arrays and DddA halves. To develop efficient ZF-DdCBEs, including for in vivo applications, DdCBE architecture, ZF scaffolds, and DddA deaminase components were comprehensively engineered. This v7 architecture supports a 10-fold average improvement in mitochondrial base editing efficiency over an initial v1 architecture that simply replaced TALE repeat arrays in DdCBE with ZF arrays, and a >3.6-fold average improvement over ZFDs in side-by-side comparisons. Four strategies were identified to minimize off-target editing caused by spontaneous split DddA reassembly, and these approaches were integrated to engineer high-specificity ZF-DdCBE variants with minimal off-target editing and efficient on-target editing of mitochondrial or nuclear DNA. Their compact size enables ZF-DdCBEs to be delivered with a single AAV in vivo in mice, resulting in efficient mitochondrial base editing in the heart, liver, and skeletal muscle. ZF-DdCBEs enable compact, all-protein in vitro and in vivo base editing for the precise editing of nuclear or organelle DNA without double-strand DNA breaks.

### Architecture Engineering to Optimize ZF-DdCBE On-Target Activity

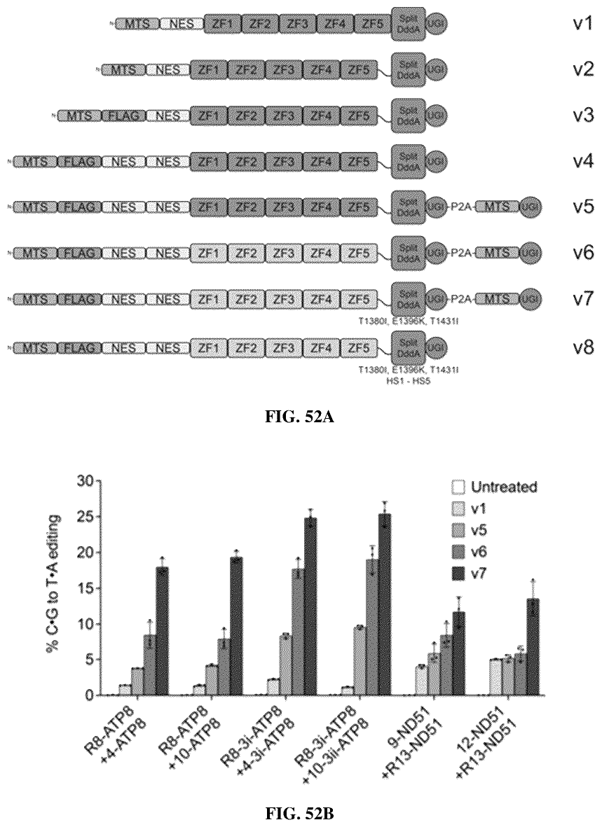

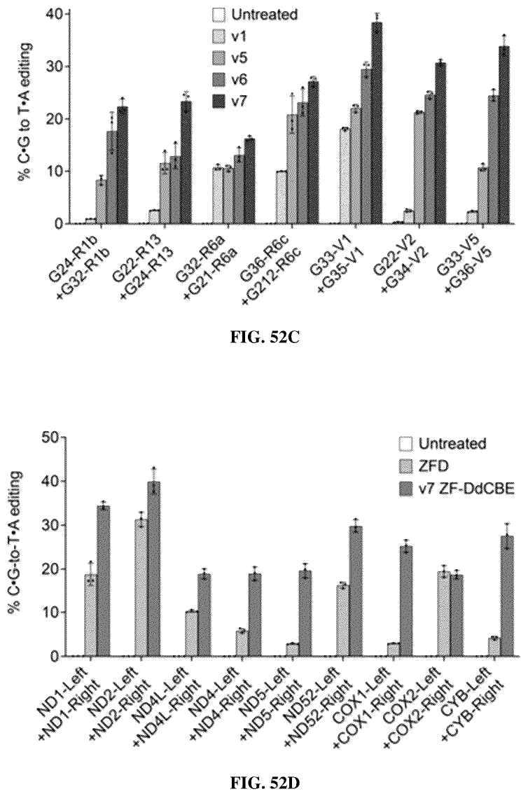

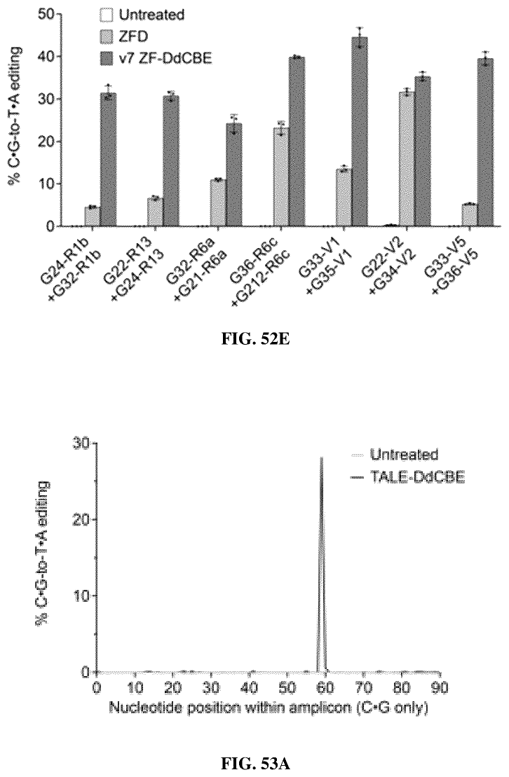

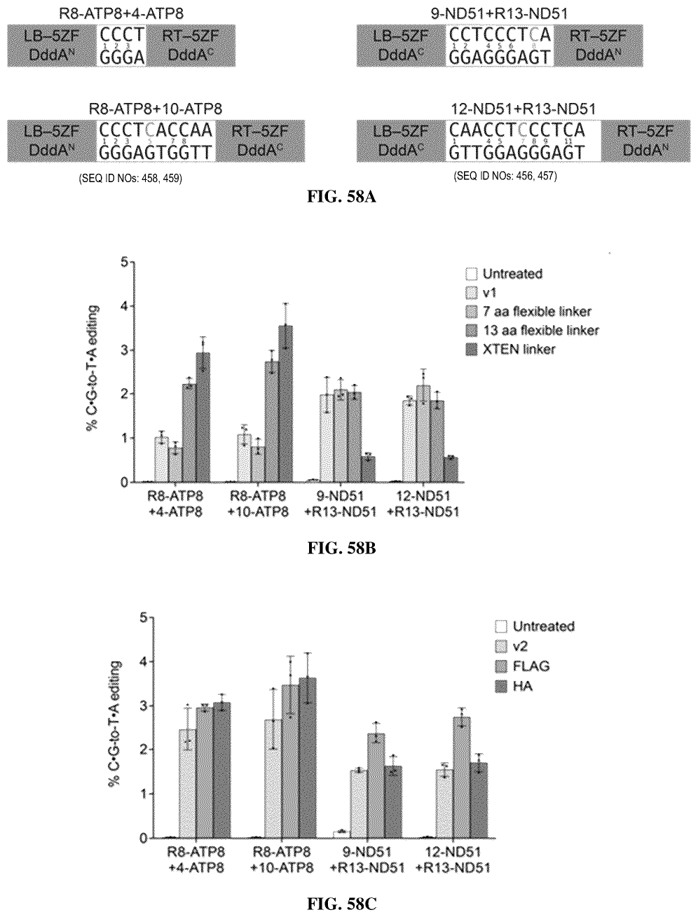

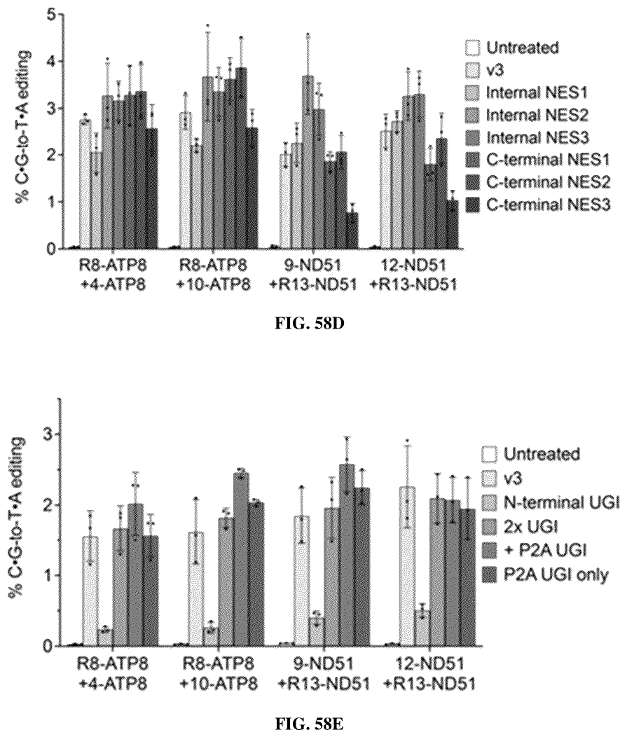

The initial ZF-DdCBE architecture (designated v1) was based on TALE-targeted DdCBEs¹ and consisted of a five-ZF (5ZF) array preceded by a mitochondrial targeting signal (MTS) from the human ATP5F1B gene and a nuclear export signal (NES) from MVM NS2 as previously reported for mitochondrially targeted ZF nucleases (mtZFNs)^{26, 27}, followed by a two-amino acid linker, one split DddA half, and one UGI (FIG. 52A). To target sites in human mtDNA, a previously characterized 5ZF array from the literature was used to form one half of a ZF-DdCBE pair²⁶, and two 5ZF arrays were designed following the modular assembly approach^{28, 29} that each formed the other half of a ZF-DdCBE pair. Using a total of six 5ZF arrays, this resulted in two ZF-DdCBE pairs targeting the mitochondrial ATP8 gene and two ZF-DdCBE pairs targeting the mitochondrial ND5 gene with 4-, 10-, 9-, and 12-bp spacing regions containing TC dinucleotides, respectively (FIG. 58A). The ZF-DdCBE pairs defined herein are named A+B where A and B specify the left and right ZF, respectively. While iterated ZF selection approaches are considered to yield ZF arrays with higher target binding activity and specificity^{30, 31}, the simpler modular assembly approach was chosen to determine if a highly accessible ZF design strategy readily available to most researchers could support ZF-DdCBEs. The simplest model for ZF binding assumes each ZF repeat within a ZF array behaves as an independent DNA-binding module that targets adjacent, discrete trinucleotide sequences. Models taking into account target site overlap (TSO) effects instead consider each ZF repeat within a ZF array as targeting overlapping four nucleotide sequences, which confers certain target sequence requirements^{66, 67}. Rather than restrict the design of ZF arrays only to sequences that satisfy these second-order TSO effects, trinucleotide modular assembly was chosen as the most user-friendly ZF design strategy available to most researchers. Additional ZF array iterated selection or screening strategies that accommodate target sequence context dependencies offer additional performance benefits, but with additional resource and experimental requirements⁶⁸⁻⁷⁰.

When expressed in human HEK293T cells following plasmid transfection, this v1 ZF-DdCBE architecture resulted in base editing efficiencies ranging from 1-2% for four ZF-DdCBE pairs tested across two sites (FIG. 58B). These results establish that ZF-DdCBEs can be constructed using ZF arrays in place of TALE repeats and can successfully install targeted C-to-T edits in mitochondria in living cells, albeit with very low initial activity. These v1 ZF-DdCBEs were used as the starting point for development and optimization.

ZF-DdCBE editing outcomes might be limited if the linker between the ZF array and the split DddA deaminase constrained access of reassembled DddA to the target nucleotide(s). The two-amino acid linker in architecture v1 was replaced with a 7- or 13-amino acid Gly/Ser-rich flexible linker, or a 32-amino acid XTEN linker. Across the four ZF-DdCBE pairs tested, using a 13-amino acid Gly/Ser-rich flexible linker supported the greatest improvements in editing efficiency, on average increasing editing efficiency 1.7-fold over v1 ZF-DdCBEs (FIG. 58B). This architecture was designated v2 (FIG. 52A).

Suboptimal cellular localization of ZF-DdCBEs might impair editing outcomes if they are transported into mitochondria inefficiently or remain partially localized in the nucleus. Since the mitochondrial import efficiency of a protein depends on its local structure adjacent to the MTS³², an unstructured epitope (a FLAG or HA tag) was introduced immediately downstream of the MTS as previously reported for mtZFNs²⁶ in an effort to improve ZF-DdCBE mitochondrial import. Across the four ZF-DdCBE pairs tested, inserting a FLAG tag led to an average improvement in editing efficiency over v2 of 1.5-fold (FIG. 58C). This architecture was designated v3 (FIG. 52A).

To minimize the fraction of ZF-DdCBE that was localized to the nucleus in order to maximize organelle editing efficiency, the effect of adding an additional NES from HIV-1 Rev, MAPKK, or MVM NS2 to v3 ZF-DdCBEs, either downstream of the existing internal NES or at the C-terminus of the protein, was tested. Across the four ZF-DdCBE pairs tested, inserting an additional internal NES from MAPKK led to an average improvement in editing efficiency of 1.4-fold over v3 (FIG. 58D). This architecture was designated v4 (FIG. 52A).

Next, it was investigated whether incomplete inhibition of mitochondrial base excision repair could be limiting ZF-DdCBE editing efficiency. To test if different UGI positioning or copy number could enhance mitochondrial base editing efficiency, the location of UGI within the fusion protein was moved to a position N-terminal of the 5ZF array, and a second copy of UGI was appended to the C-terminus, or a separate mitochondrially targeted UGI was expressed in trans using a self-cleaving P2A peptide (with or without removing the C-terminally fused UGI). Across the four ZF-DdCBE pairs tested, expressing an additional copy of MTS-UGI in trans led to an average improvement in editing efficiency over v3 of 1.3-fold (FIG. 58E). Combining this improvement with the v4 architecture to create v5 resulted in editing efficiency on average 3.4-fold over that of v1 ZF-DdCBEs across the four ZF-DdCBE pairs tested (FIGS. 52A-52B). Collectively, these data show that ZF-DdCBE editing efficiency can be substantially improved compared to the initial v1 architecture by increasing the linker length between the ZF array and split DddA, improving mitochondrial import, enhancing nuclear export, and further suppressing residual cellular UDG activity.

### Effects of ZF Array Length and Composition on ZF-DdCBE Performance

Next, optimal ZF arrays for ZF-DdCBEs were investigated. Natural ZF arrays are found in transcription factors that localize to the nucleus and contain cryptic nuclear localization signals (NLSs) present within the ZF fold^{33, 34}. Cycling of nuclear import and export mediated by competition between NLS and NES motifs may impede localization of ZF-DdCBEs to the mitochondria and therefore limit mitochondrial base editing. It was reasoned that shorter ZF arrays with fewer NLS-containing ZF repeats would exhibit weaker nuclear localization and therefore may support higher mitochondrial editing efficiency due to improved mitochondrial localization.

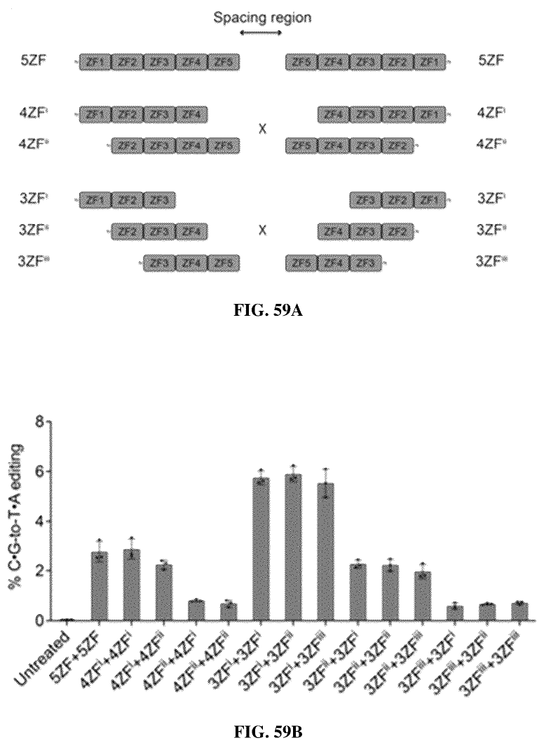

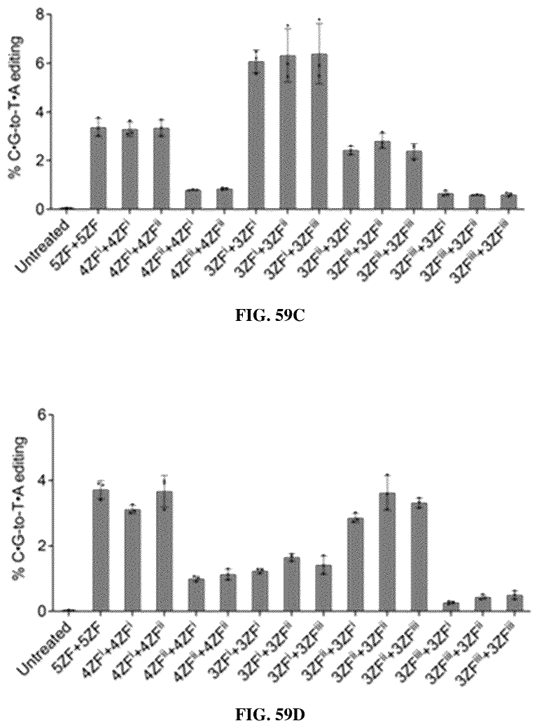

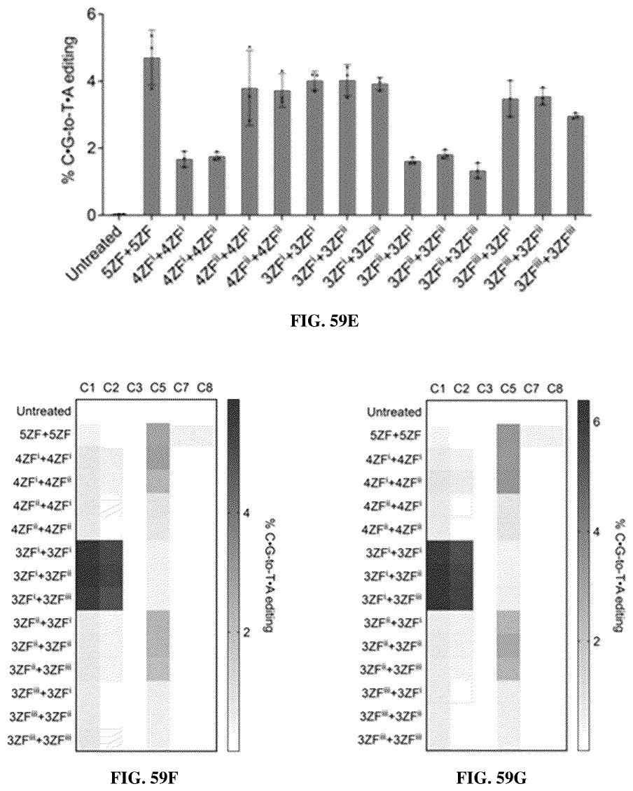

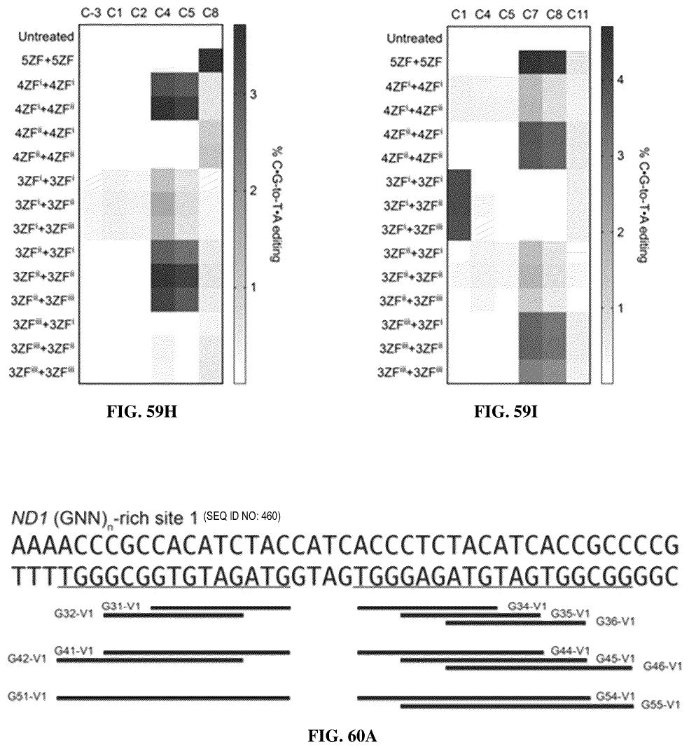

To understand the effects of ZF array length on ZF-DdCBE editing efficiency, first each 5ZF was truncated to create a set of two 4ZFs and a set of three 3ZFs by removing either one or two individual ZFs, respectively (FIG. 59A). The resulting four 4ZF+4ZF combinations and nine 3ZF+3ZF combinations were tested in the context of ZF-DdCBEs derived from each of the original four ZF-DdCBE pairs (FIGS. 59B-59I). For each of the ZF-DdCBE pairs, ZF truncation affected both the editing efficiency and the position of the target nucleotide(s) that are edited within the spacing region. In general, it was observed that ZF-DdCBEs containing shorter ZFs exhibited lower editing efficiency, however six 3ZF+3ZF combinations with substantially higher editing efficiencies than their parent 5ZF+5ZF pairs were identified despite using shorter ZF arrays. These data suggest that ZF arrays as short as 3ZF are sufficient to mediate efficient mitochondrial C•G-to-T•A base editing, and that the precise location of the ZF binding site, and therefore deaminase positioning, strongly influences which target bases are edited most efficiently.

While longer ZF arrays generally support more potent DNA binding and higher editing efficiencies, ZF-DdCBEs containing 3ZF arrays can offer sufficient binding specificity to be useful for target-specific mitochondrial editing. On average, a recognition sequence of only 7 or 8 bp can specify a unique site in the 16,569-bp human mitochondrial genome, whereas a recognition sequence of at least 16 bp is required to specify a unique site in the human nuclear genome. Therefore, longer ZF arrays are required to confer sufficient sequence specificity when targeting loci within nuclear DNA sequences. However, longer ZF arrays may also bind tightly to related off-target sequences. Long ZF arrays may bind to truncated or mismatch-containing binding site sequences without much reduction in binding affinity, which could undermine their targeting specificity. Arrays with four or more ZFs have the potential to bind to off-target sites using subsets of three fingers⁷¹. In contrast, shorter ZF arrays are expected to be more sensitive to mutations in their binding site because if there is a mismatch, the binding affinity is expected to fall more rapidly⁷². Within a 3ZF array, the suboptimal binding of any individual ZF repeat would more strongly compromise the overall binding affinity of the protein to a mismatched sequence than for a longer ZF array in which a suboptimal binding interaction of any individual ZF can be better compensated for.

The binding affinity of extended ZF arrays can vary widely, and the combined binding strength of shorter ZF arrays linked together in tandem is not generally considered an additive effect^{71, 73, 74}. Therefore the choice of ZF array length for mitochondrial ZF-DdCBEs is expected to be a balance between maximizing on-target editing and minimizing off-target editing and should be determined by the researcher on a case-by-case basis.

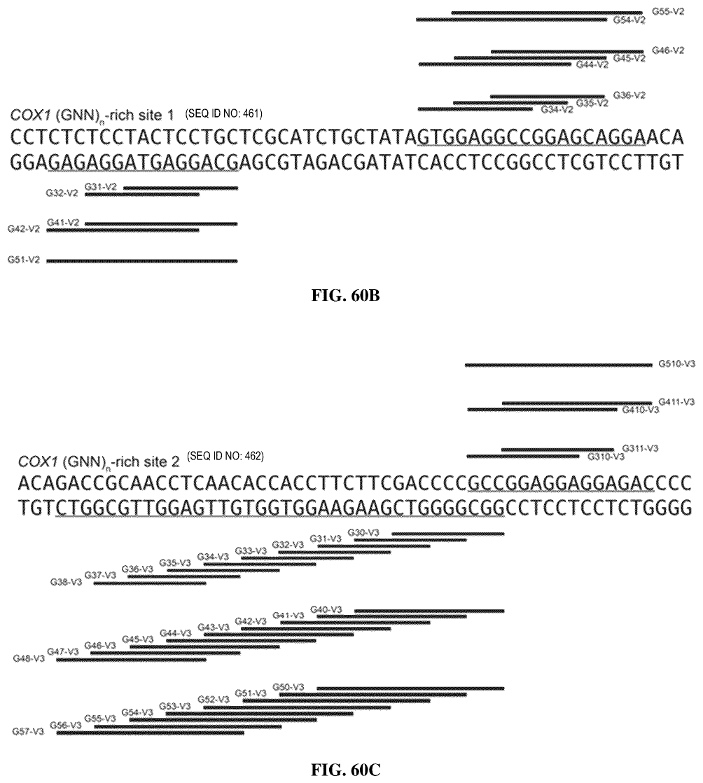

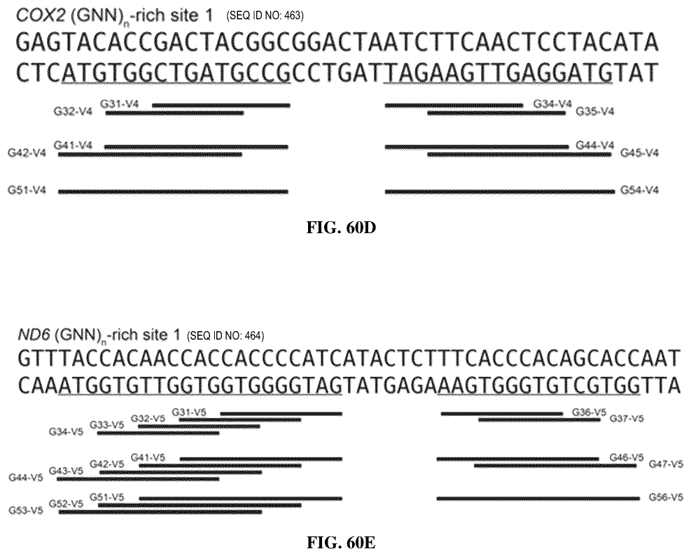

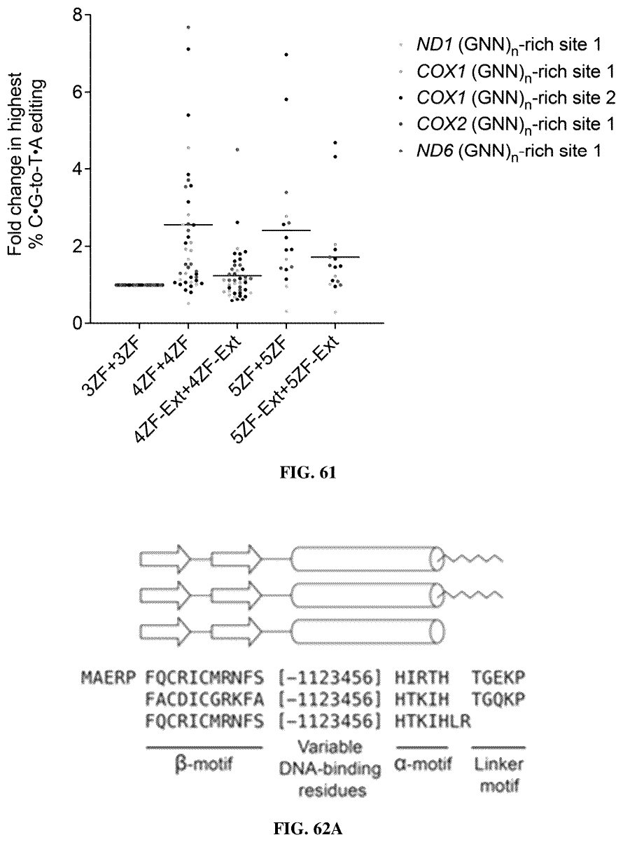

To investigate the effects of ZF array length more systematically, five sites were identified within human mtDNA that comprise a TC-containing spacing region flanked by sequences consisting exclusively of (GNN)ₙ trinucleotides. (GNN)ₙ-rich sites were selected because ZFs containing GNN-binding modules were predicted to have a higher binding affinity, on average, than ANN, TNN, or CNN-binding modules³⁵. Therefore, testing ZFs containing exclusively GNN-binding modules may minimize variability in binding affinity when designing ZF arrays by modular assembly. At each site, a panel of 3ZFs were designed that could be extended outwards away from the spacing region to create longer 4ZF or 5ZF arrays that all shared the same split DddA positioning and therefore maintained a fixed spacing region, enabling a direct comparison (FIGS. 60A-60E). 42 ZF-DdCBEs containing 3ZF+3ZF pairs were tested, and their performance was compared against 42 4ZF+4ZF and 16 5ZF+5ZF pairs (FIG. 61). The results indicated that on average, longer ZF arrays correlated with increased editing efficiency, with 4ZF+4ZF pairs and 5ZF+5ZF pairs leading to an average 2.6- and 2.4-fold improvement relative to 3ZF+3ZF pairs, respectively.

The effects of including an extended linker following ZF3 (the third ZF repeat) in 4ZF and 5ZF arrays, which have been reported to reduce DNA-binding strain in longer ZF arrays³⁶⁻³⁹, were also investigated. The editing efficiency achieved by 42 4ZF+4ZF and 16 5ZF+5ZF ZF-DdCBE pairs were compared against their counterparts in which an extended linker was incorporated into each ZF array (FIG. 61). It was found that 4ZF and 5ZF arrays designed using exclusively canonical linkers supported higher editing efficiencies on average, and therefore extended linkers were not used in subsequent designs.

### Defining New ZF Scaffolds Improves ZF-DdCBE Performance

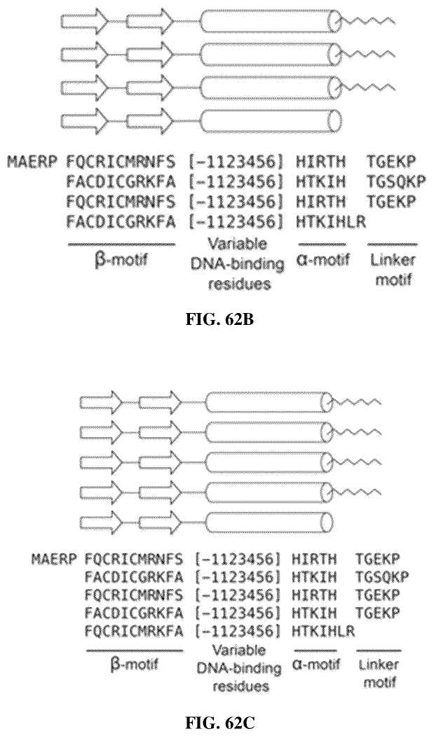

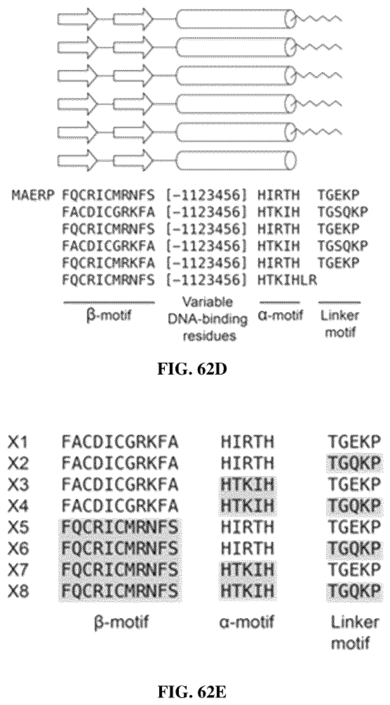

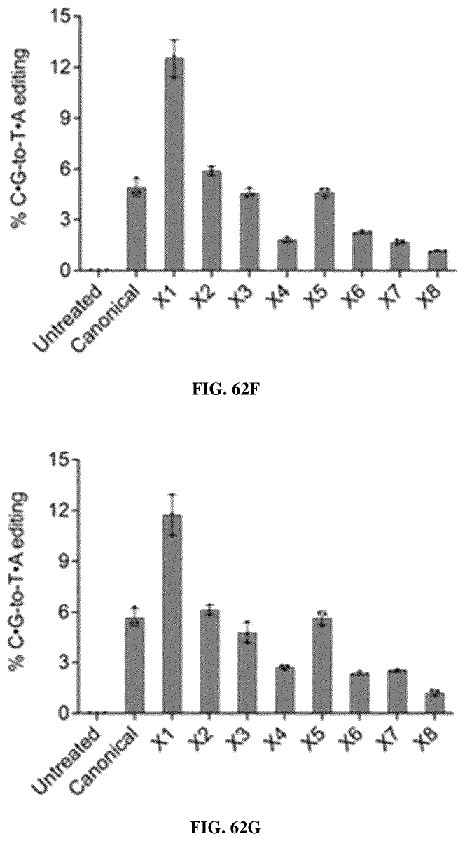

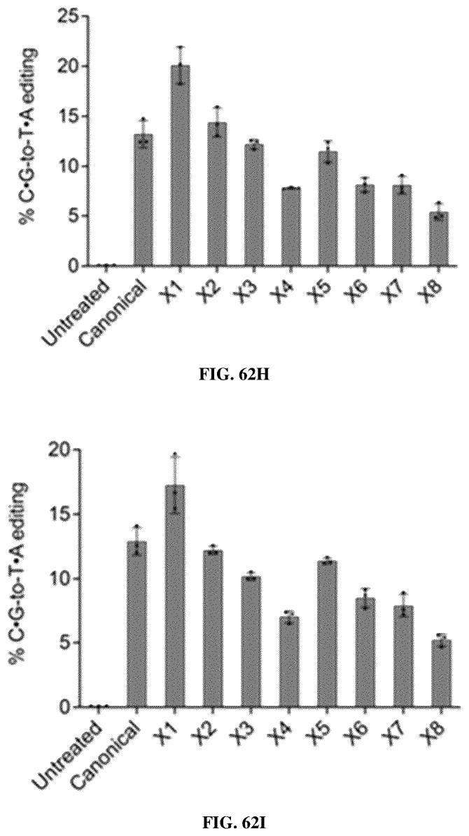

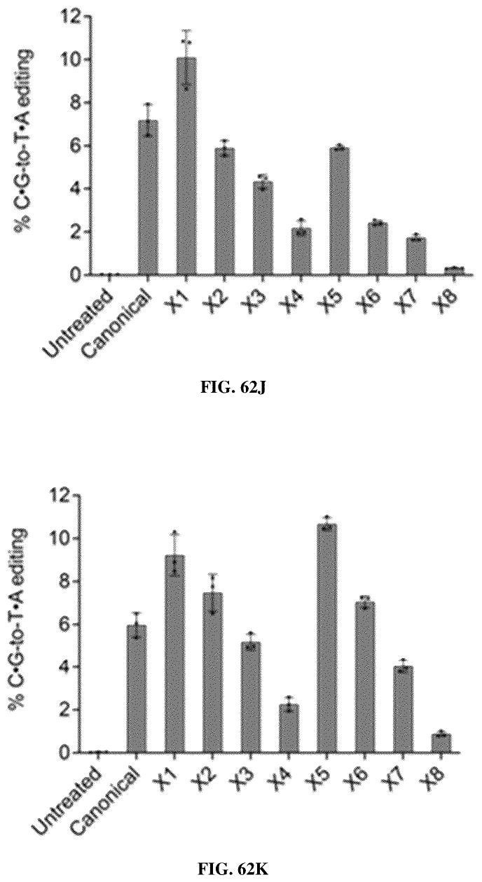

Next, alternative ZF scaffolds were sought that might improve ZF-DdCBE editing efficiency by enhancing DNA-binding affinity or reducing the strength of the inherent cryptic NLS sequences that form part of the ZF fold. Each ZF repeat within a ZF array is linked together by short flexible linkers and consists of a beta-sheet motif, seven variable DNA-binding residues, and an alpha-helical motif. As defined herein, a ZF scaffold consists of a beta-motif, an alpha-motif, and a flexible linker motif, independent of the DNA-binding residues that specify the targeted trinucleotide DNA sequence. The sequences of the beta-motif, alpha-motif, and flexible linker motif vary between individual ZF repeats within both natural and designed ZF arrays (FIGS. 62A-62D). ZF-DdCBE editing efficiency could potentially be improved by eliminating this sequence variation to create ZF arrays composed of identical repeating scaffolds exclusively containing motif sequences with superior performance. A set of eight new ZF scaffolds were therefore defined, named X1-X8, and used these to create ZF arrays in which every ZF repeat shared an identical scaffold sequence. These eight scaffold sequences represent all possible combinations of the two beta-motifs, two alpha-motifs, and two linker motifs found in canonical ZNF268-derived ZFs⁴⁰ (FIG. 62E). Across six ZF-DdCBE pairs of length varying from 3ZF to 5ZF tested at two target sites, scaffold X1 conferred an average of 1.7-fold improvement relative to the canonical ZNF268-derived scaffold (FIGS. 62F-62K). These observations demonstrated that ZF scaffold engineering can create ZF-DdCBEs with higher editing efficiency across different sites and different ZF array lengths.

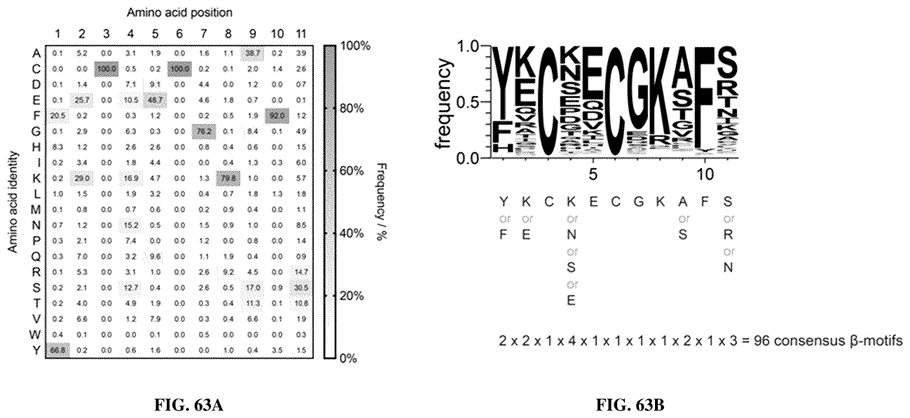

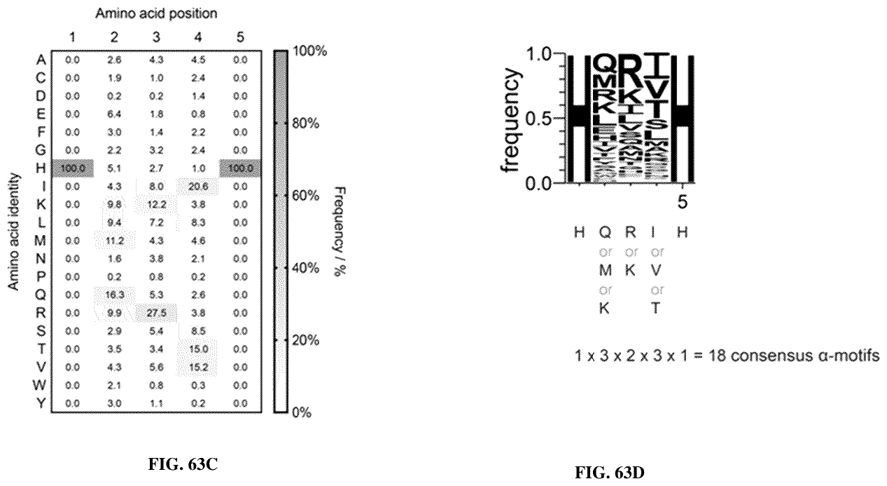

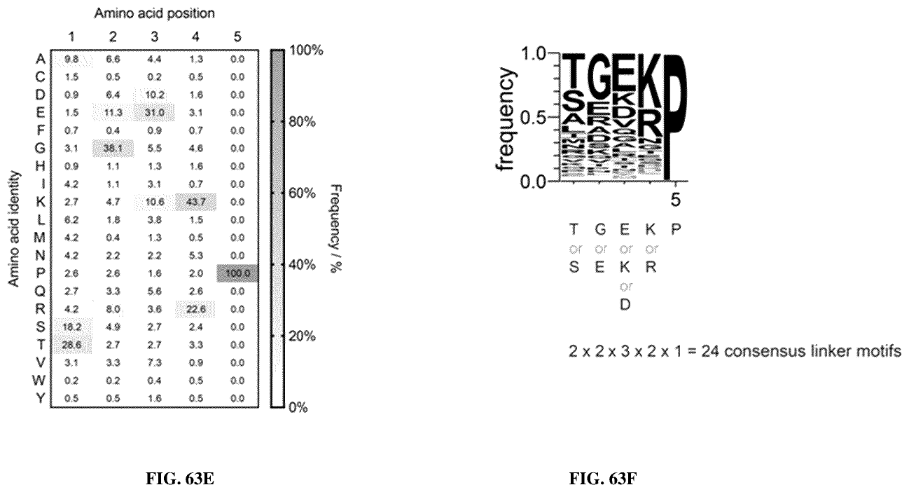

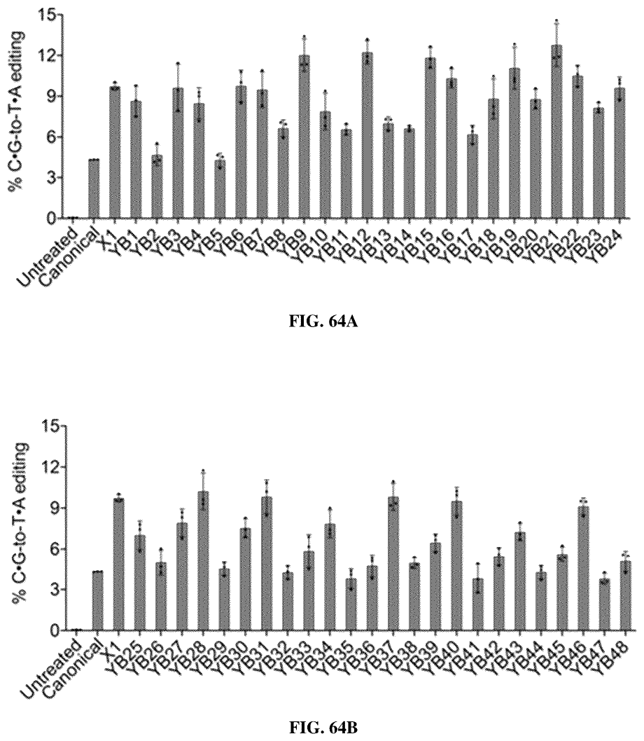

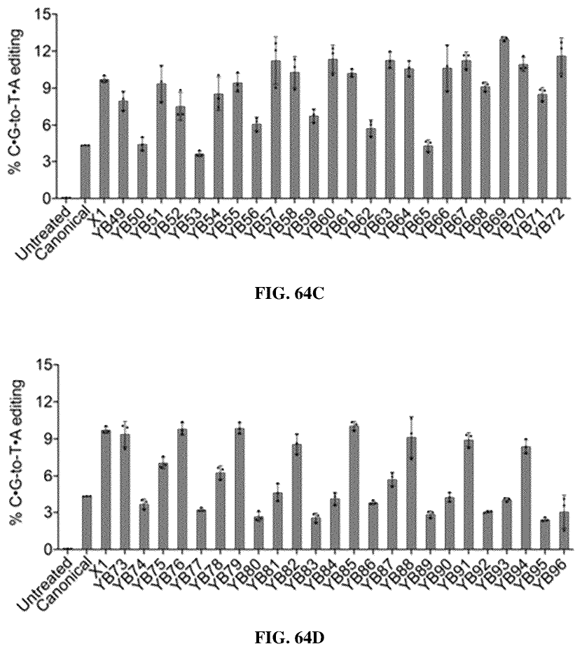

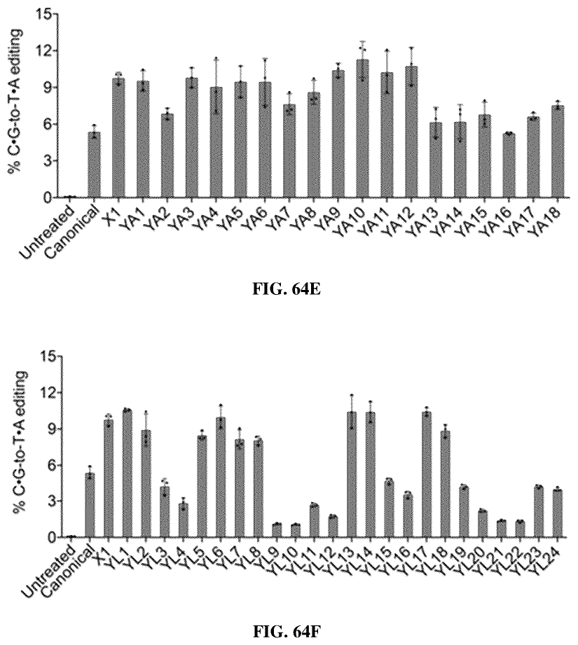

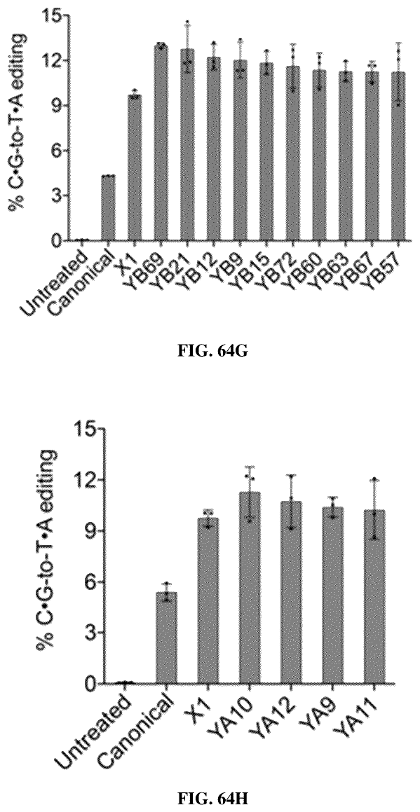

To explore whether other ZF scaffold sequences can confer even higher base editing activity to ZF-DdCBEs than canonical ZNF268-derived sequences, natural ZF diversity was searched for additional ZF scaffolds. The human proteome was searched for ZF-containing sequences, and 3,356 unique beta-motifs, 625 unique alpha-motifs, and 549 unique linker motifs were identified. Amino acid frequencies were calculated at each position within the motifs, and these were used to define 96 consensus beta-motifs, 18 consensus alpha-motifs, and 24 consensus linker motifs based on the most common amino acids at each position (FIGS. 63A-63F). ZF-DdCBE variants were constructed based on the X1 scaffold in which every ZF within the 5ZF array was replaced with either the beta-motif only, alpha-motif only, or the linker motif only with one of the new consensus motifs. Testing these ZF-DdCBE pairs revealed a new beta-motif that conferred a 1.3-fold increase in editing over the X1 scaffold (FIGS. 64A-64D, and 64G) and a new alpha-motif that conferred a 1.2-fold increase over the X1 scaffold (FIGS. 64E and 64H). No new linker motifs were found that outperformed the X1 scaffold (FIGS. 64F and 64I).

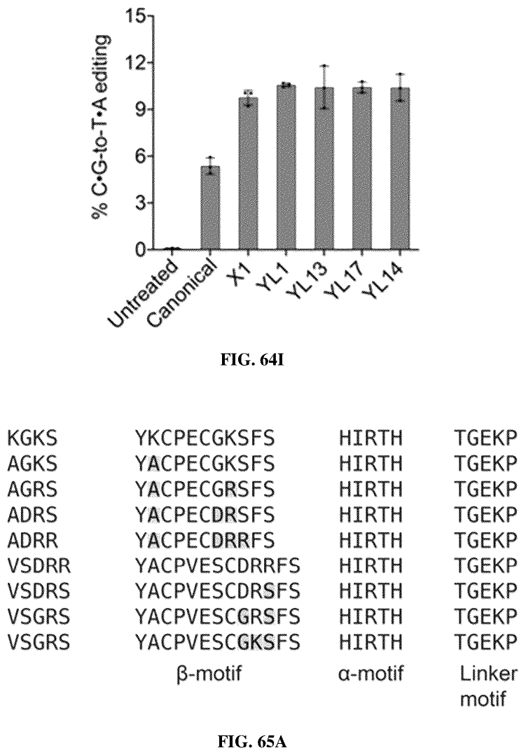

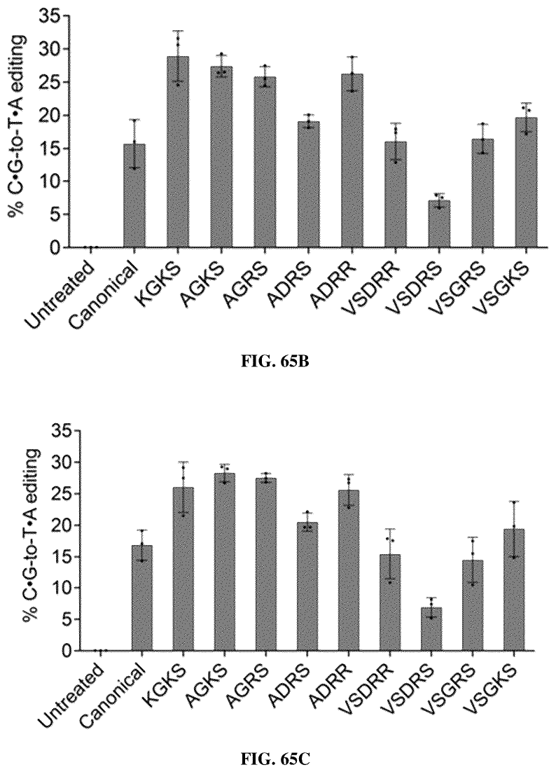

By combining the best-performing beta-motif and alpha-motif, a new ZF scaffold V20 and variant V2 were defined. A new ZF scaffold AGKS derived from the human transcription factor Sp1C that showed increased editing efficiency over X1 was also defined (FIGS. 65A-65C). There was sequence similarity between the beta-motifs in ZFN268(F1) and Sp1C, YACPVESCDRRFS (SEQ ID NO: 342) and YKCPECGKSFSQK (SEQ ID NO: 1087) respectively. These sequences differ by the insertion of two residues in addition to four substitutions. A set of nine beta-motifs were designed in which the sequences were progressively mutated to incrementally revert the ZFN268(F1) beta-motif towards the Sp1C beta-motif and vice versa (FIG. 65A). v5 ZF-DdCBE variants were constructed based on the X1 scaffold in which only the beta-motif was changed and two ZF-DdCBE pairs were tested to determine if any of these new ZF scaffold sequences could improve editing efficiency. Compared to the canonical ZNF268-derived scaffold, scaffold AGKS conferred an increase in editing efficiency of 1.7-fold across the two pairs tested (FIGS. 65B-65C). Scaffold AGKS was included in the set of optimized ZF scaffolds.

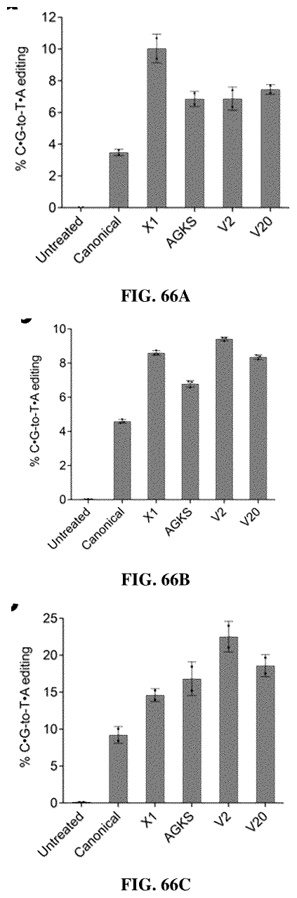

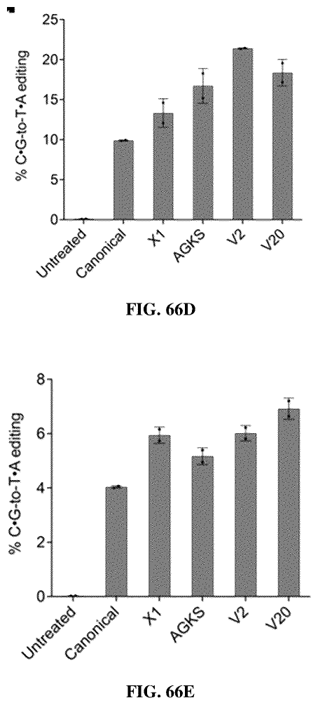

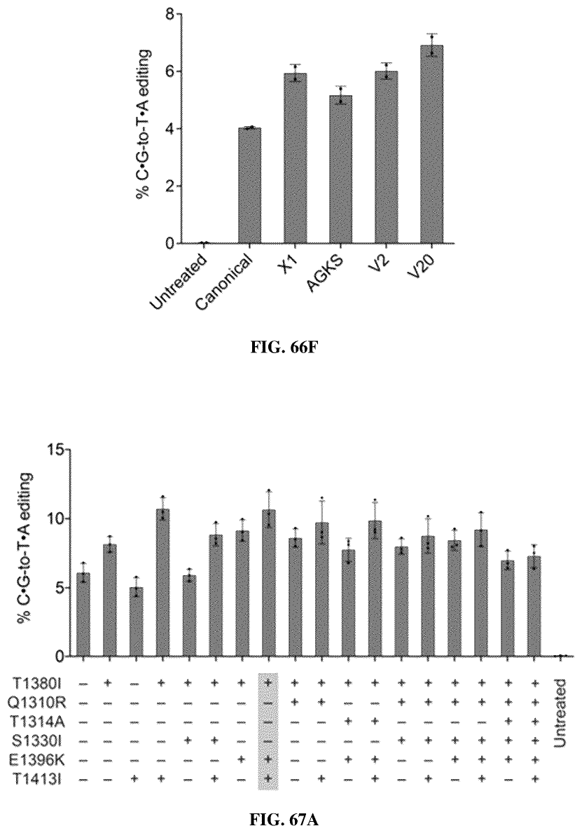

This set of four new ZF scaffolds (X1, V2, V20, and AGKS) was tested using six ZF-DdCBE pairs at two sites (FIGS. 66A-66F). For each ZF-DdCBE pair tested, editing efficiency was improved compared to the canonical ZNF268-derived scaffold for all four new ZF scaffold variants. Selecting the best-performing ZF scaffold for each pair led to an average 2.2-fold improvement over the canonical ZNF268-derived scaffold. This change was combined with v5 architecture to create v6 (FIG. 52A). Across the six ZF-DdCBE pairs tested, v6 on average increased base editing efficiency 6.6-fold over v1 and 2.0-fold over v5 (FIG. 52B). These results collectively establish that ZF-DdCBE base editing efficiency can be enhanced by optimizing the design of ZF arrays used for DNA targeting.

### Introducing DddA Mutations Enhances ZF-DdCBE Base Editing Efficiency

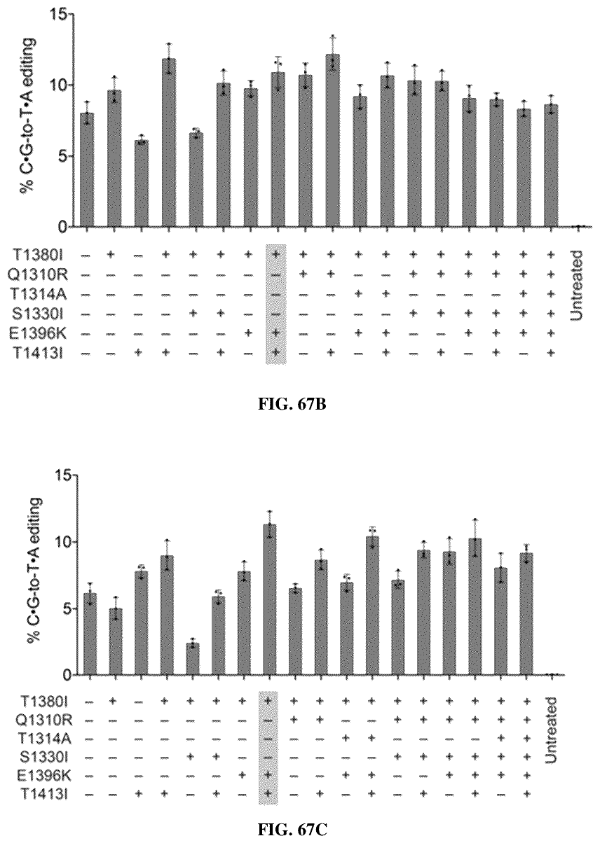

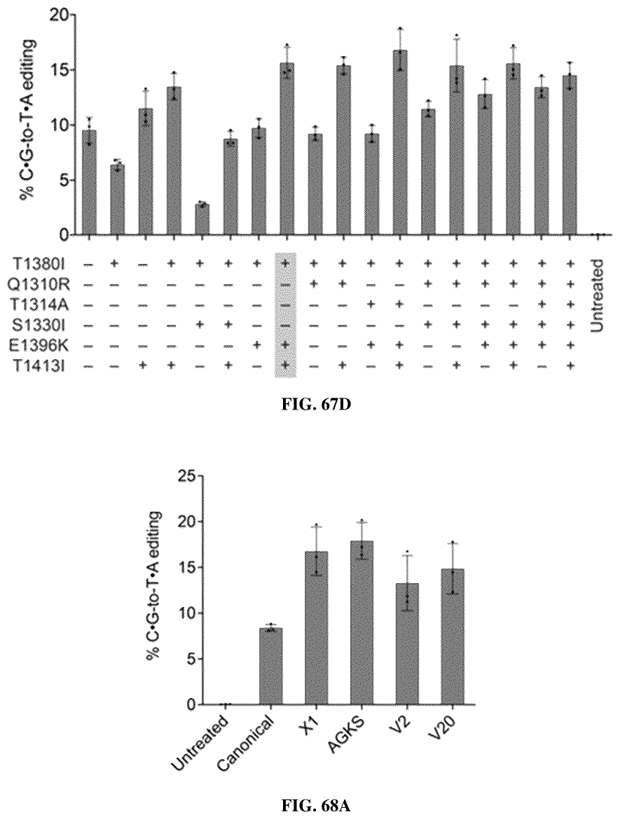

As a final strategy to optimize the architecture and sequence of ZF-DdCBEs for on-target editing efficiency, mutations in DddA deaminase were tested for their ability to enhance ZF-DdCBE editing. Phage-assisted continuous evolution (PACE) has been used to evolve DddA deaminase variants that support improved TALE-based DdCBE activity². To test if evolved DddA mutations improve ZF-DdCBEs, combinations of Q1310R, T1314A, S1330I, T1380I, and E1396K in DddA^{N} were assayed with and without T1413I in DddA^{C} (FIGS. 67A-67D). Across the four ZF-DdCBE pairs tested, the triple mutant T1380I, E1396K, T1413I led to an average improvement in editing over canonical DddA of 1.6-fold. These mutations were combined with v6 architecture to create v7 (FIG. 52A). These results suggest that using a more active DddA variant can improve ZF-DdCBE editing outcomes.

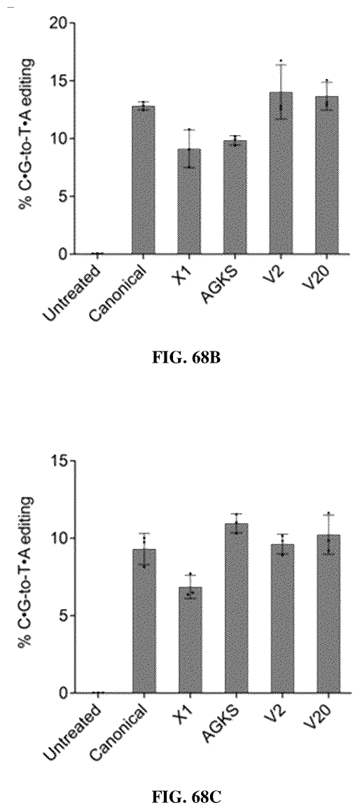

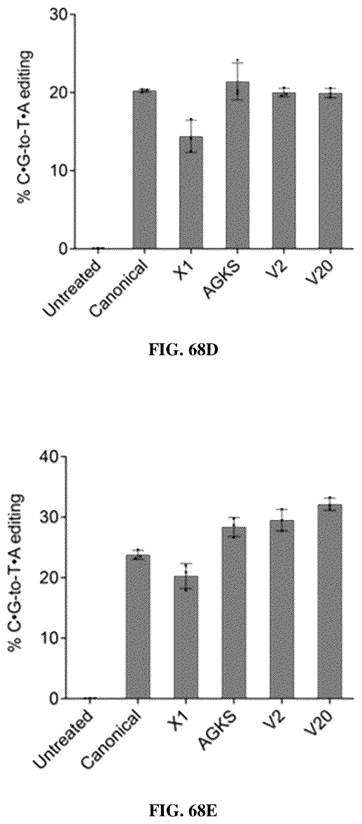

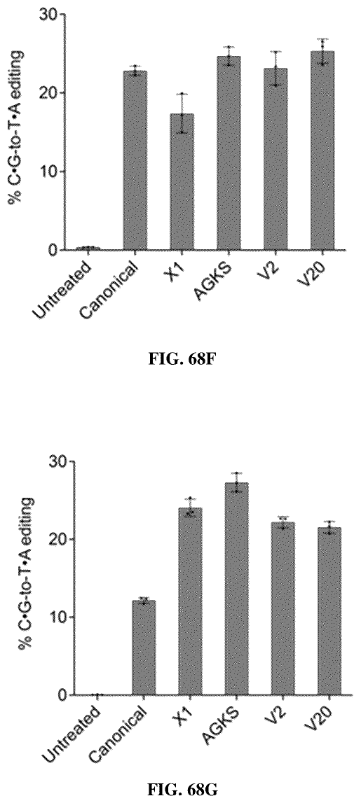

To validate the ZF-DdCBE optimizations, the v1, v5, v6, and v7 architectures were re-tested at the original set of six ZF-DdCBE pairs at two sites. Across these six pairs, v7 ZF-DdCBEs achieved an average of 11-fold higher editing over v1 (FIG. 52B). To demonstrate that these architectural improvements are generalizable to ZF-DdCBEs targeting any sites across mtDNA, seven new ZF-DdCBE pairs targeting seven different sites across four genes were tested, and the v1, v5, v6, and v7 architectures were compared (FIG. 52C, FIGS. 68A-68G). Across these seven pairs, v7 ZF-DdCBEs achieved an average of 9.5-fold higher editing relative to v1.

For six of these seven pairs, one half of the ZF-DdCBE pair uses an N-terminal ZF-DdCBE architecture in which split DddA is fused N-terminally to the ZF array, while the other half of the ZF-DdCBE pair uses a canonical C-terminal fusion of split DddA. Importantly, N-terminal fusions of split DddA with TALE repeat arrays do not result in efficient DdCBEs, thus requiring that TALE-DdCBE halves must target opposite DNA strands, whereas the compatibility of ZF-DdCBEs with N-terminal or C-terminal split DddA fusions provides researchers with the flexibility to design ZF-DdCBE pairs that bind either the same or opposite DNA strands around the target nucleotide(s), resulting in additional targeting options not available to TALE-DdCBEs. Collectively, these findings integrate optimized architectures, improved ZF scaffolds, DddA activity-enhancing mutations, and split DddA fusion orientation flexibility to enhance the editing efficiency of compact all-protein base editors.

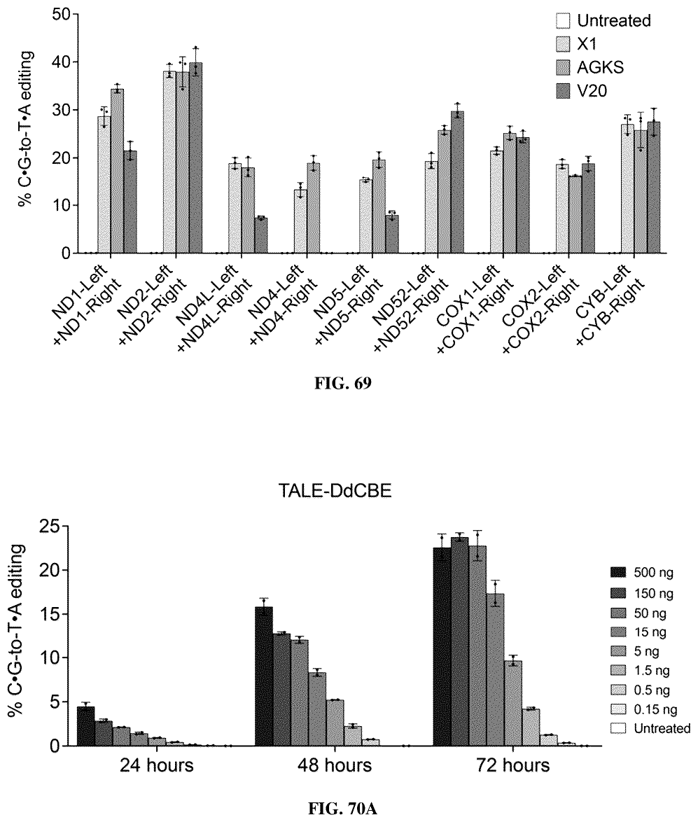

To directly compare the performance between previously reported ZFDs²⁵ with that of optimized ZF-DdCBEs, nine mtDNA-targeting ZFD pairs were converted into the v7 ZF-DdCBE architecture and X1, AGKS, and V20 ZF scaffolds were tested. Across the nine sites tested, the best-performing ZF scaffold for each pair led to an average 3.6-fold improvement in on-target editing efficiency for ZF-DdCBEs compared to ZFDs (FIG. 52D and FIG. 69). In addition, a separate set of seven optimized v7 ZF-DdCBEs were converted into ZFD architectures, and their relative performance was tested at editing mitochondrial sites. The optimized ZF-DdCBEs led to an average 3.9-fold higher on-target editing efficiency compared to ZFDs across the seven pairs tested (FIG. 52E). Collectively, these side-by-side comparison data at 16 distinct mtDNA target sites suggest that the more extensively optimized ZF-DdCBEs offer substantially higher on-target editing efficiencies than ZFDs.

### Characterizing Off-Target Editing by ZF-DdCBEs

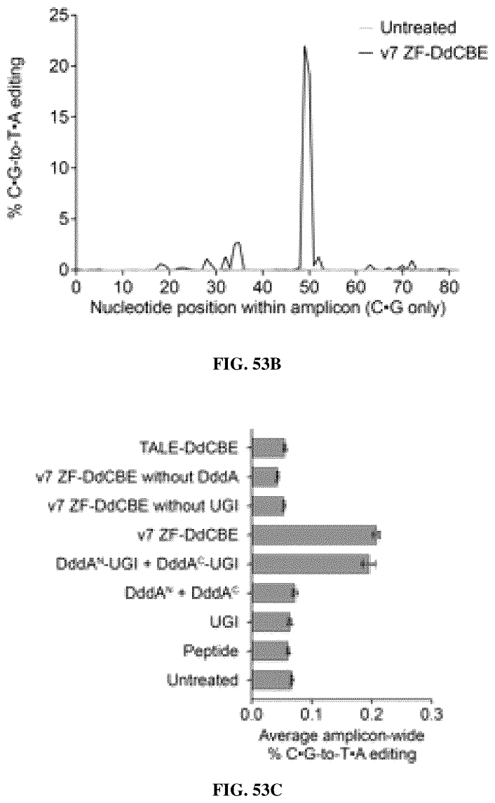

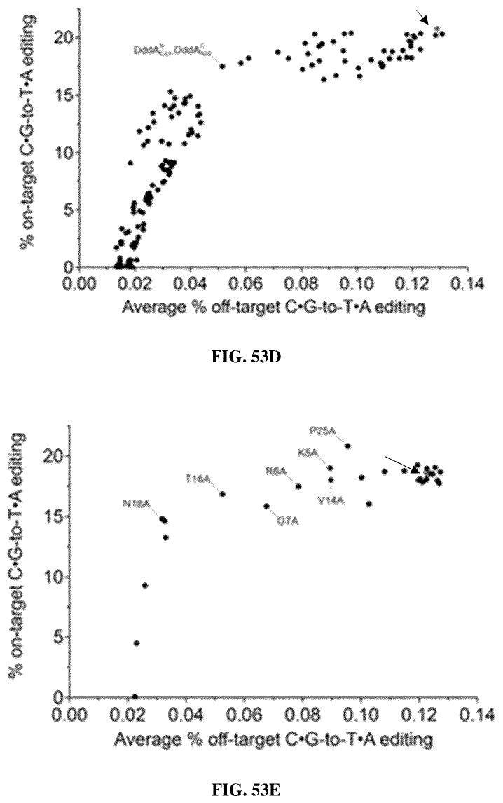

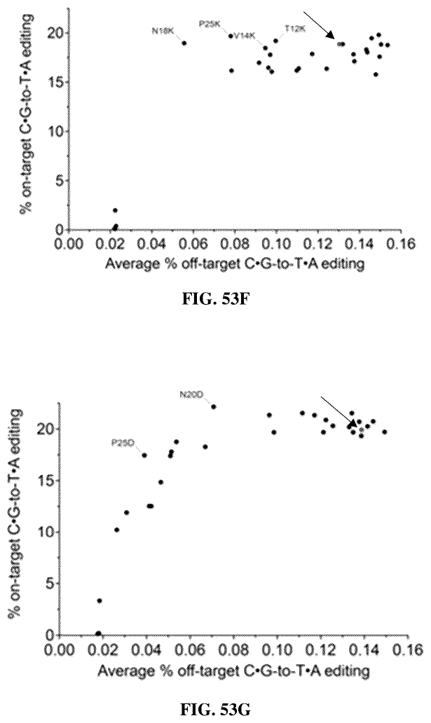

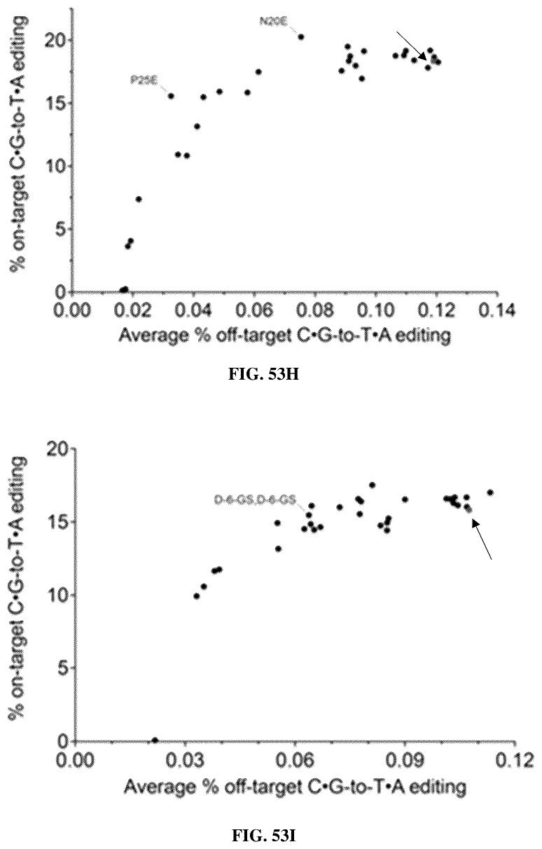

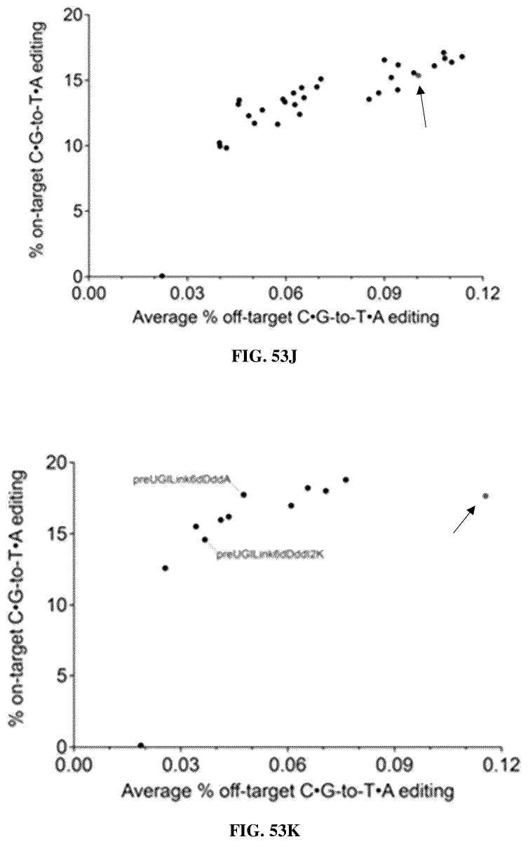

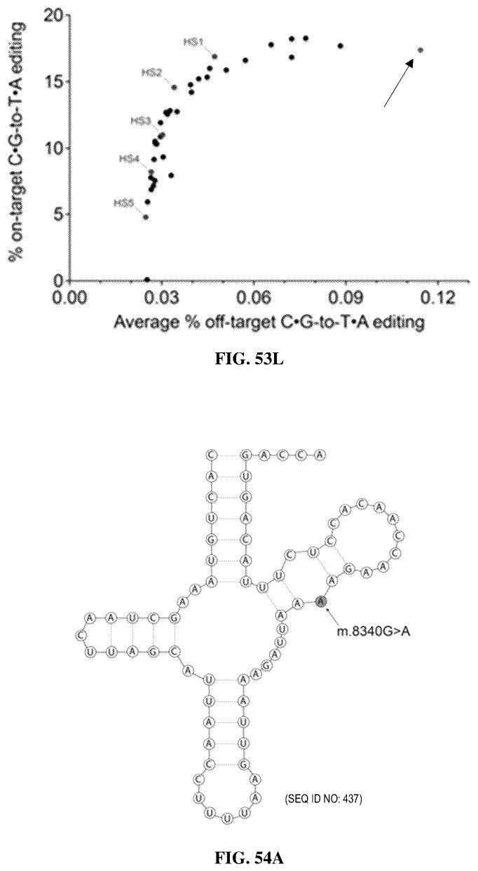

Amplicon-wide (˜200 bp) sequencing data was compared for a high-performing TALE-based DdCBE pair¹ and a v7 ZF-DdCBE pair, both targeting sites in mtDNA. Efficient on-target editing (28%) and very low frequencies of off-target editing was observed for the TALE-based DdCBE pair (typically ≤0.2% C•G-to-T•A conversion at each off-target nucleotide in the amplicon), but much higher off-target editing of up to 2% at C•G base pairs scattered across the amplicon for the v7 the ZF-DdCBE pair (FIGS. 53A-53B). These results suggest that ZF-DdCBEs introduce a higher level of off-target edits than TALE-based DdCBEs.

To investigate if the higher level of off-target editing activity exhibited by ZF-DdCBEs arises from spontaneous DddA reassembly, from ZF-dependent DddA reassembly, or both, individual components of the v7 ZF-DdCBE architecture were delivered into mitochondria (FIG. 53C). Targeted amplicon sequencing was used to initially assess mtDNA-wide off-target editing activity. Transfected HEK293T cells expressing an inactive mitochondrially targeted short peptide as a negative control did not exhibit any detectable editing compared to untreated cells. Cells expressing mitochondrially targeted UGI also did not display any editing above background (FIG. 53C), demonstrating that the endogenous mutational load arising from spontaneous deamination is very low.

Cells expressing mitochondrially localized DddA^{N}-UGI and DddA^{C}-UGI displayed non-targeted editing, while cells expressing mitochondrially localized DddA^{N} and DddA^{C} did not (FIG. 53C). These results suggest that the spontaneous reassembly of split DddA halves is sufficient to give rise to untargeted deaminase activity, recapitulating the native-like activity of the full-length DddA toxin. While the natural base-excision repair (BER) pathway endogenous to mitochondria can adequately repair C-to-U deamination caused by DddA reassembly, when mitochondrial uracil BER is suppressed by UGI, C•G-to-T•A conversions are observed.

Delivering a representative v7 ZF-DdCBE increased off-target editing compared to expression of DddA^{N}-UGI and DddA^{C}-UGI without ZFs, indicating a ZF-dependent component of off-target editing (FIG. 53C). Removal of either UGI or the split-DddA from the ZF-DdCBE architecture abolished detectable off-target editing. Collectively, these results indicate that ZF-DdCBE off-target editing arises from spontaneous association of the DddA split halves under conditions of suppressed uracil BER by UGI, and that the inclusion of a ZF array can increase off-target editing.

ZF-DdCBE off-target editing could thus proceed via three different paths: (i) dual ZF-dependent off-target editing in which both ZF-DdCBE halves bind to off-target DNA sequences in close spatial proximity; (ii) single ZF-dependent off-target editing in which a single ZF-DdCBE protein binds to off-target DNA sequences and transiently recruits the other DddA half; or (iii) ZF-independent off-target editing in which the two DddA split halves spontaneously reassemble without requiring ZF binding. Weakening the interaction between the DddA split halves could reduce single ZF-dependent and ZF-independent off-target editing, without necessarily impairing on-target editing efficiency.

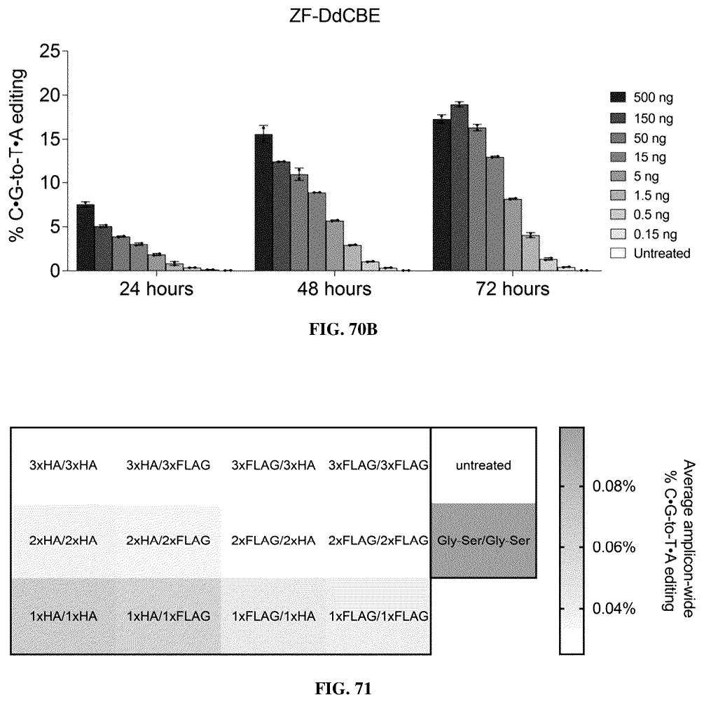

It was previously reported that delivery into mitochondria of DddA^{N}-UGI and DddA^{C}-UGI preceded by 3×HA tag and 3×FLAG tag sequences, respectively, gave rise to no detectable C•G-to-T•A conversion above background¹. In contrast, the delivery of both DddA^{N}-UGI and DddA^{C}-UGI each preceded by a Gly/Ser-rich flexible linker produced measurable C-to-T editing in mtDNA (FIG. 53C). To test whether the amino acid sequences immediately upstream of DddA^{N} and DddA^{C} could be modulated to change the level of editing activity observed, the preceding Gly/Ser-rich flexible linker was systematically replaced with sequences containing increasing numbers of negatively charged HA or FLAG tag motifs. The non-targeted editing activity decreased as the total negative charge density increased (FIG. 71). These results suggest that destabilization of the interaction between the split DddA halves can reduce off-target editing caused by spontaneous reassembly of DddA.

### Engineering High-Specificity ZF-DdCBEs

These findings suggested several strategies to minimize ZF-DdCBE off-target editing by reducing the binding affinity between the split DddA halves. First, truncation of DddA^{N} and DddA^{C} or shifting the position of the split site within DddA may weaken the ability of the DddA halves to spontaneously reassemble in the absence of target DNA co-binding. Second, introducing point mutations into DddA^{C} might destabilize the binding affinity between the DddA halves and reduce their spontaneous association. Third, increasing electrostatic repulsion between DddA^{N} and DddA^{C} by introducing negatively charged residues upstream or downstream of DddA^{N} and DddA^{C} may also impede target-independent reassembly. Fourth, fusion of a catalytically inactivated DddA^{N} might outcompete spontaneous reassembly of DddA^{N} with DddA^{C} in the absence of target-templated co-localization. Each of these strategies was tested using a 3ZF+3ZF v7 ZF-DdCBE pair (ATP8-R8-3i+4-3i) targeting the mitochondrial ATP8 gene in HEK293T cells and high-throughput amplicon sequencing to detect on-target and off-target editing.

### DddA Truncation to Enhance ZF-DdCBE Specificity

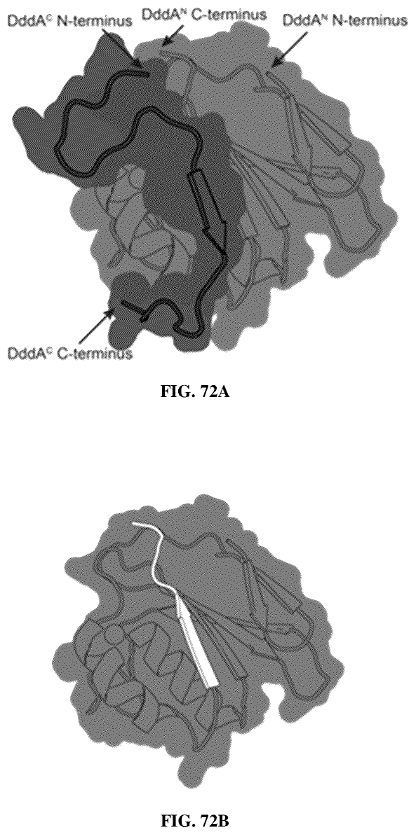

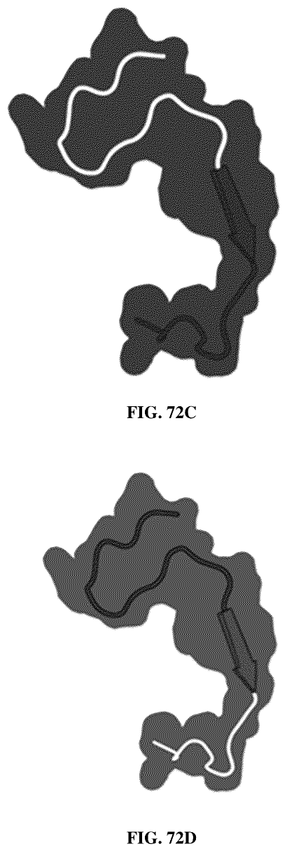

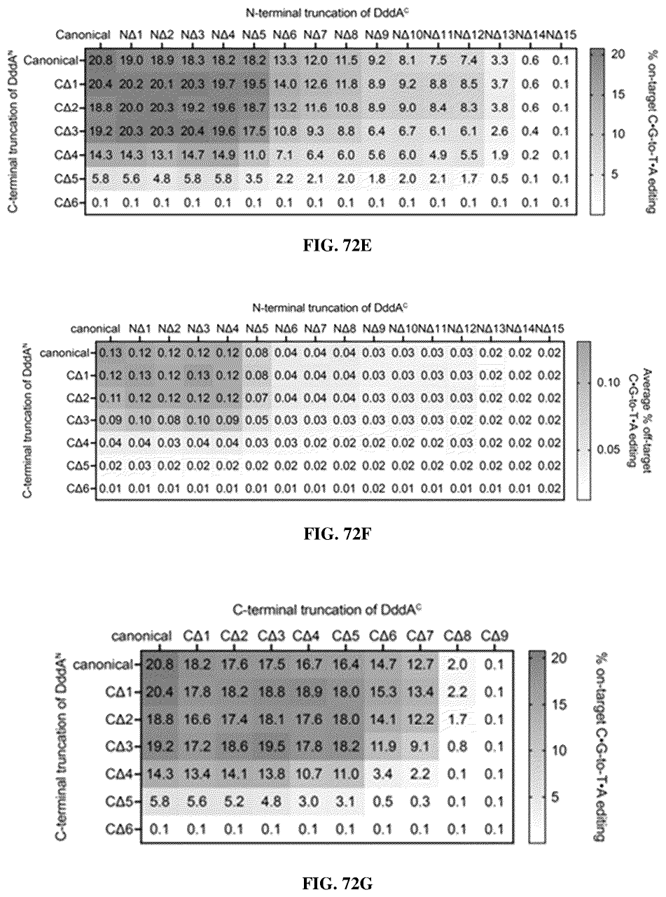

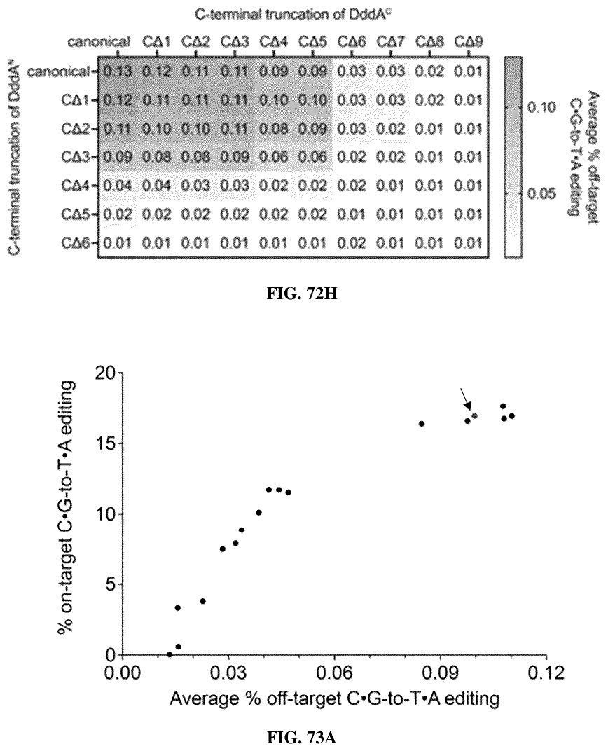

First, the effects of DddA^{N} and DddA^{C} truncation on ZF-DdCBE performance was explored. A series of ZF-DdCBE constructs were created in which DddA^{N} was incrementally C-terminally truncated by 1 to 6 residues and designated DddA_{CΔ1}^{N} to DddA_{CΔ6}^{N}. A series of ZF-DdCBE constructs in which DddA^{C} was either incrementally truncated at its N-terminus by 1 to 15 residues, designated DddA_{NΔ1-15}^{C}, or incrementally truncated at its C-terminus by 1 to 9 residues, designated DddA_{CΔ1-9}^{C} was also created (FIGS. 72A-72D). A matrix of ZF-DdCBE pairs encompassing all 175 possible combinations of one half of a ZF-DdCBE pair carrying canonical DddA^{N} or DddA_{CΔ1-6}^{N}, and the second half of a ZF-DdCBE pair carrying either canonical DddA^{C}, DddA_{NΔ1-15}^{C} or DddA_{CΔ1-9}^{C} were tested. Decreases in on-target editing upon C-terminal truncation of DddA^{N} by more than five residues, N-terminal truncation of DddA^{C} by more than 14 residues, or C-terminal truncation of DddA^{C} by more than eight residues was observed (FIGS. 72E and 72G). Importantly, shorter truncations displayed a smooth, gradual decrease in on-target editing concomitant with a faster decline in off-target editing (FIGS. 72F and 72H). These data were visualized in an XY-plot (FIG. 53D), and combinations that were left-shifted from the canonical ZF-DdCBE pair (reflecting lower off-target editing) while remaining as high on the Y-axis as possible (reflecting high on-target editing) were identified. The combination of DddA_{CΔ3}^{N} with DddA_{NΔ5}^{C} conferred a 3.1-fold reduction in off-target editing accompanied by only a 1.2-fold reduction in on-target editing compared to the canonical ZF-DdCBE pair. These results demonstrate that truncation of the split DddA halves can reduce ZF-DdCBE off-target editing while maintaining efficient on-target editing.

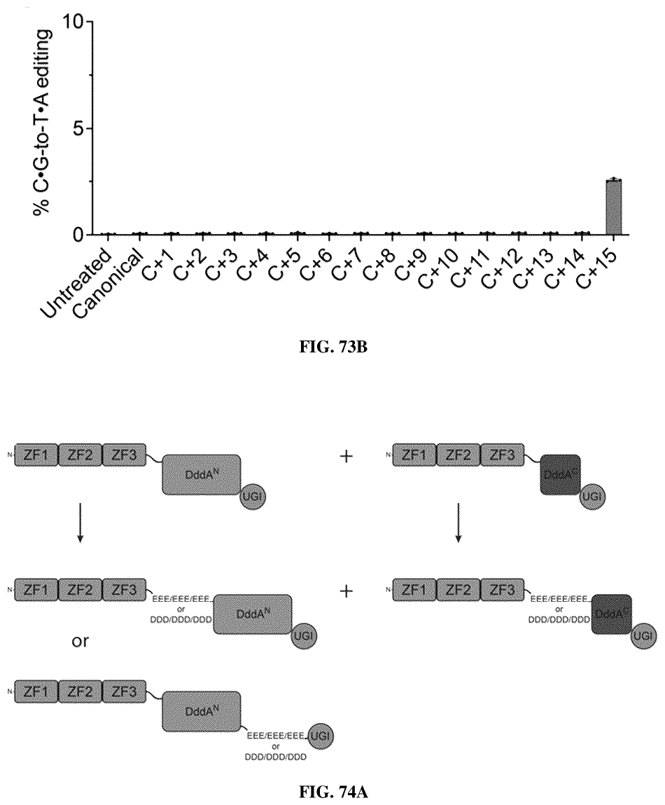

As an alternative or addition to truncating DddA^{N} and DddA^{C} to reduce ZF-DdCBE off-target editing, the effects of shifting the position of the canonical G1397 split site within DddA to create split DddA halves with a longer DddA^{N} and a shorter DddA^{C} were also investigated, but better results than can be achieved by truncation alone were not observed (FIGS. 73A-73B).

As an alternative to truncating DddA^{N} and DddA^{C} to reduce ZF-DdCBE off-target editing, the effects of shifting the position of the canonical G1397 split site within DddA to create split DddA halves with a longer DddA^{N} and a shorter DddA^{C} were investigated. A series of ZF-DdCBE pairs were tested in which DddA^{N} was incrementally extended at its C-terminus by between one and 15 residues, designated DddA_{C+1}^{N} to DddA_{C+15}^{N}, while at the same time DddA^{C} was incrementally truncated at its C-terminus by between 1 and 15 residues, designated DddA_{NΔ1-15}^{C} (FIG. 73A). The best combination (Ddd_{C+5}^{N} with DddA_{NΔ5}^{C}) exhibited a 1.2-fold reduction in off-target editing while retaining 97% of on-target editing relative to the canonical ZF-DdCBE pair. These results suggest that shifting the position of the split site can alter the ratio of on-target to off-target editing performance of ZF-DdCBEs, but this approach does not yield ZF-DdCBEs with a specificity profile better than can be achieved by truncation. The split halves DddA_{C+1}^{N} to DddA_{C+14}^{N} remained inactive by themselves by transfecting only a single ZF-DdCBE half carrying a DddA^{N} variant, and no detectable base editing in the absence of a DddA^{C} variant was observed (FIG. 73B). Additionally, DddA_{C+15}^{N} displayed base editing activity, signifying that C-terminal truncations of DddA of greater than 16 amino acids were required to abolish DddA deaminase activity.

### Installing DddA Point Mutations to Enhance ZF-DdCBE Specificity

Second, point mutations were introduced into DddA^{C} in an effort to weaken the binding association between DddA^{N} and DddA^{C}. A series of 28 ZF-DdCBE constructs conducting Ala scanning mutagenesis across each position within DddA^{C} were tested (FIG. 53E). Mutations such as K5A, R6A, G7A, T9A, V14A, T16A, N18A, and P25A led to reductions in off-target editing compared to canonical DddA^{C}, with or without only modest reductions in on-target editing. In particular, N18A and P25A reduced average off-target editing by 10.6-fold and 1.4-fold, while retaining 80% or 112% of on-target editing compared to canonical DddA^{C}, respectively.

Since Ala point mutations represent the deletion of side-chain interactions compared to the canonical protein, the introduction of actively destabilizing mutations might further weaken the binding affinity between split DddA halves and reduce ZF-DdCBE off-target editing through a different mechanism. To investigate the effects of introducing positively charged residues into DddA^{C}, a series of 27 ZF-DdCBE constructs conducting Lys scanning mutagenesis across each position within DddA^{C} were tested (FIG. 53F). Mutations T12K, V14K, N18K, and P25K each reduced off-target editing compared to canonical DddA^{C}, with or without only modest reductions in on-target editing. For example, N18K reduced average off-target editing by 3.2-fold while retaining the same on-target editing as canonical DddA^{C}.

Next, it was investigated whether introducing a negatively charged mutation into DddA^{C} might reduce ZF-DdCBE off-target editing differently to positively charged mutations. A series of 59 ZF-DdCBE constructs conducting either Glu or Asp scanning mutagenesis across each position within DddA^{C} were tested (FIGS. 53G-53H). The results identified the best-performing mutations as N20D, N20E, P25D, and P25E. For example, P25D reduced average off-target editing by 5.6-fold while retaining 88% of on-target editing compared to canonical DddA^{C}. Collectively, these results suggested that introducing mutations into DddA^{C} that weaken the association between DddA^{N} and DddA^{C} can reduce off-target editing by ZF-DdCBEs while maintaining efficient on-target editing.

### Introducing Negative Charge at the Termini of DddA to Enhance ZF-DdCBE Specificity

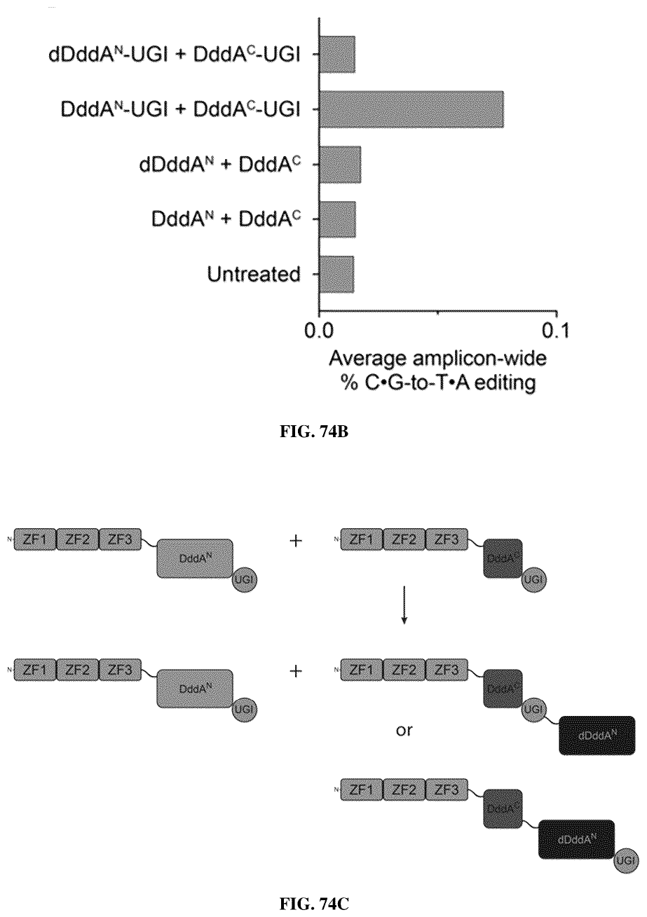

As a third approach to decreasing ZF-DdCBE off-target editing, negatively charged residues were introduced upstream or downstream of the split DddA halves to increase electrostatic repulsion and weaken their association. The G1397 split site in DddA was predicted to position the C-terminus of DddA^{N} and the N-terminus of DddA^{C} adjacent upon heterodimerization. In addition, the N-termini of DddA^{C} and DddA^{N} were predicted to be in close proximity (FIG. 72A). Split DddA variants were created in which the three, six, or nine residues in the 13-amino acid Gly/Ser-rich flexible linker upstream of DddA^{N} and DddA^{C} were mutated to either Glu or Asp residues (FIG. 74A). Variants were also created in which three, six, or nine Glu or Asp residues were inserted into the Gly/Ser-rich flexible linker downstream of DddA^{N}. Sixty different ZF-DdCBE pairs with increasing levels of electrostatic repulsion were tested, and combinations that improved target specificity were identified (FIGS. 53I-53J). For example, variant D-6-GS+D-6-GS, which has six Asp residues upstream of both DddA^{N} and DddA^{C}, reduced average off-target editing by 2.0-fold while retaining 99% of on-target editing compared to the canonical ZF-DdCBE architecture. These results demonstrated that changes to the ZF-DdCBE architecture in regions outside DddA designed to weaken the association between DddA^{N} and DddA^{C} can also be used to reduce off-target editing.

Capping with Catalytically Inactivated DddA^{N} to Enhance ZF-DdCBE Specificity

Lastly, a catalytically impaired DddA^{N} fragment localized to DddA^{C} could reduce off-target ZF-DdCBE editing by competitively inhibiting the spontaneous intermolecular reassembly of DddA^{N} and DddA^{C} in the absence of binding to adjacent DNA half-sites. First, a catalytically dead form of DddA^{N} (designated dDddA^{N}) was created by installing the E1347A mutation into DddA^{N}, and its inactivity was confirmed in HEK293T cells (FIG. 74B). Whether fusing dDddA^{N} downstream of DddA^{C} could promote dDddA^{N} and DddA^{C} association in the absence of target DNA engagement while still supporting robust on-target editing when both ZF-DdCBE pairs are localized at the target site was investigated. A series of ten ZF-DdCBE constructs were tested in which dDddA^{N} was fused downstream of DddA^{C} using Gly/Ser-rich flexible linkers of varying length, either before or after the UGI domain, and either containing or omitting the additional two mutations T1380I and E1396K (FIG. 74C). Constructs preUGILink6dDddA and preUGILink6dDddI2K reduced average off-target editing by 3.4 and 14-fold while retaining 100% and 71% on-target editing compared to canonical ZF-DdCBE architecture (FIG. 53K). The results demonstrated that C-terminal fusion of dDddA^{N} to DddA^{C} successfully produced ZF-DdCBEs with significantly reduced off-target editing profiles while maintaining efficient on-target editing. These findings validated an alternative approach to limiting ZF-DdCBE off-target editing that uses competitive inhibition between split deaminase halves rather than weakening their binding interaction.

### Combining Multiple Strategies to Reduce ZF-DdCBE Off-Target Editing

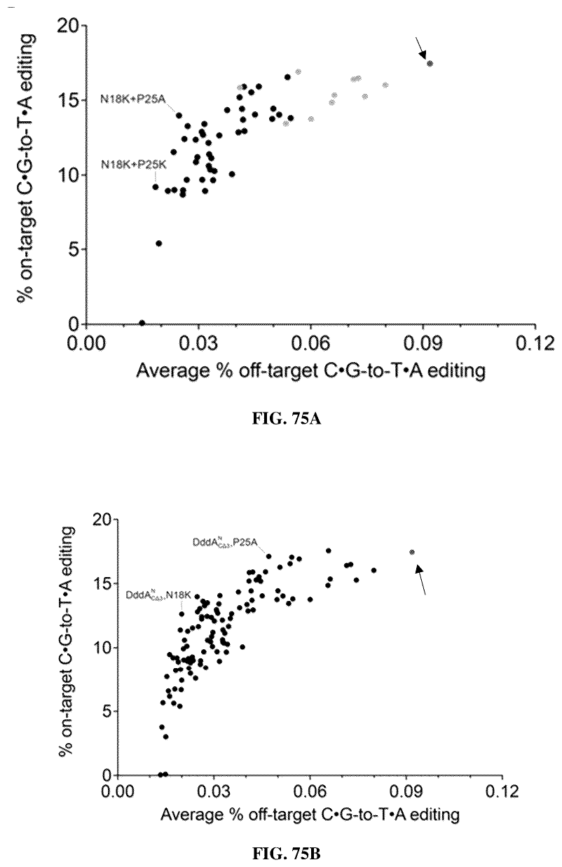

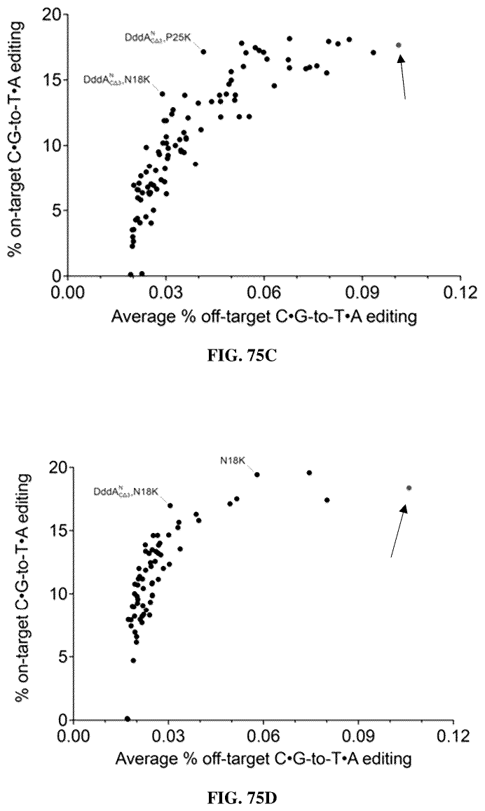

Having established four different approaches to reduce ZF-DdCBE off-target editing, it was investigated whether these approaches could be combined additively to create variants with even better specificity profiles (FIGS. 75A-75D). Having established four different approaches to reduce ZF-DdCBE off-target editing, these approaches were investigated to see if they could be combined additively to create variants with even better specificity profiles. To test the effects of combining point mutations, a set of 10 single point mutations (K5A, R6A, G7A, T9A, V14A, P25A, T12K, V14K, N18K, P25K) was selected, and all 43 pairwise combinations of double mutants were tested (FIG. 75A). To test the effects of combining point mutations and truncations, a set of eight single point mutations (G7A, T9A, V14A, P25A, T12K, V14K, N18K, P25K) was selected, and 123 different ZF-DdCBE variants comprising all possible single or double point mutations were tested either alone or in combination with the truncations DddA_{CΔ3}^{N}, DddA_{NΔS}^{C}, or both (FIGS. 75B-75C). To investigate the effects of combining any of the approaches of single point mutations, truncations, electrostatic repulsion, and dDddA^{N} capping, combinations comprising one variant from any one, two, or three of these four approaches were also tested (FIG. 75D). Collectively, these results revealed that combining more than one mutation or more than one approach not only leads to a greater reduction in off-target editing compared to using a single mutation or approach, but also a greater reduction in on-target editing. Each of these four approaches was able to create ZF-DdCBEs with improved specificity profiles.

To define a final set of high-specificity (HS) ZF-DdCBE variants, a shortlist of the top-performing single point mutations (N18K, N20E, P25A, P25K), truncations (DddA_{CΔ3}^{N}, DddA_{NΔS}^{C}), and dDddA^{N} architectures was created (preUGILink6dDddA, preUGILink13dDddA), and 35 combinations were tested for their specificity-enhancing changes (FIG. 53L). From these results, a set of five variants that offered a balance between high on-target editing and low off-target editing was selected and designated HS1 to HS5 (HS1=N18K, HS2=N18K+P25A, HS3=N18K+P25K, HS4=DddA_{CΔ3}^{N}+N18K+P25A, and HS5=DddA_{CΔ3}^{N}+N18K+P25K). HS1, HS2, HS3, HS4, and HS5 reduced average off-target editing by 4.0-, 10-, 18-, 66-fold, and down to background levels, while retaining 98%, 84%, 64%, 47%, and 27% on-target editing, respectively, compared to the canonical ZF-DdCBE pair. The HS variants selected contained only mutations and truncations that displayed a greatly improved specificity profile yet were smaller or required no increase in protein size compared to canonical ZF-DdCBEs. These HS variants were introduced into the v7 ZF-DdCBE architecture and the additional copy of mitochondrially targeted UGI expressed in trans, which was found to have minimal effect on on-target editing efficiency, was removed. These resulting high-specificity variants were designated v8^{HS1} to v8^{HS5} (FIG. 52A).

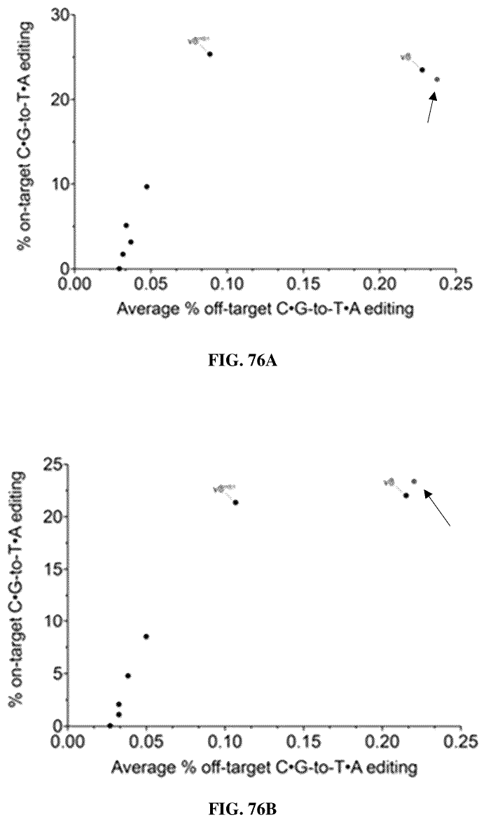

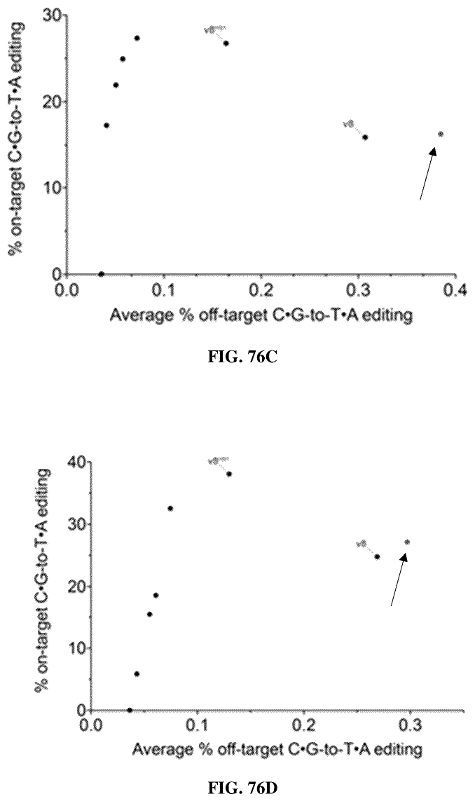

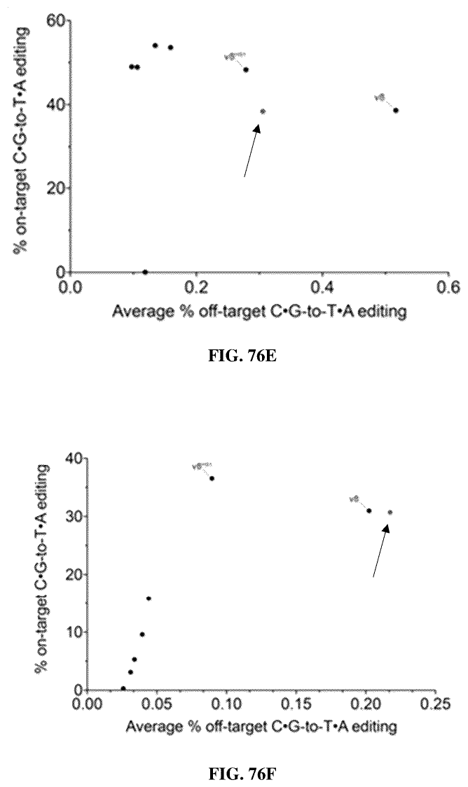

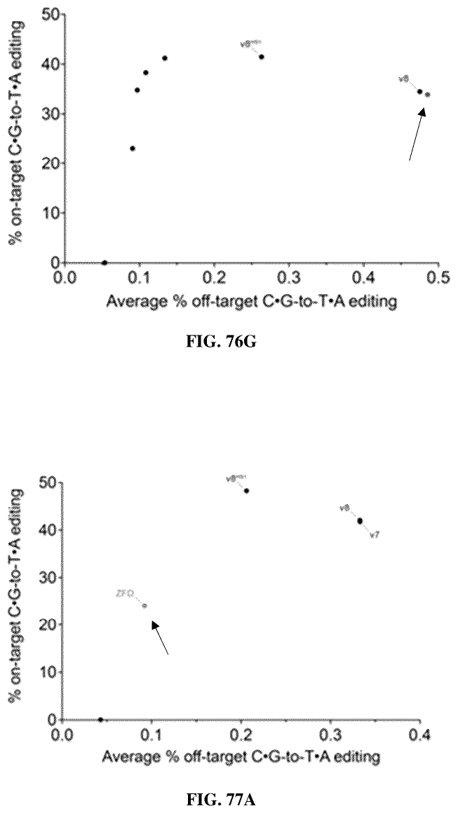

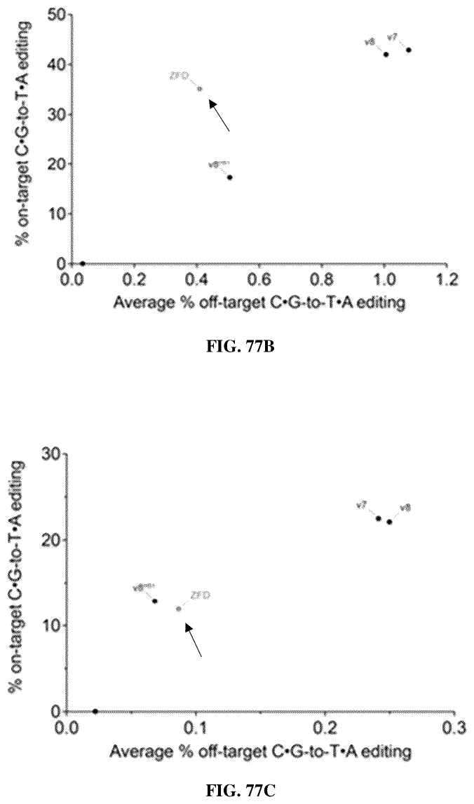

To demonstrate that these HS variant-containing v8 advancements are generally applicable to ZF-DdCBE pairs targeting any site of interest in mtDNA and are transferrable to N-terminal ZF-DdCBE architectures, all five HS variants were tested in the context of an additional eight 3ZF+3ZF v8 ZF-DdCBE pairs targeting eight different target sites across five mitochondrial genes (FIGS. 76A-76G). Six of these eight pairs featured an N-terminal ZF-DdCBE architecture in which split DddA is fused N-terminally relative to the ZF array. results showed that v8^{HS1} to v8^{HS5} reduced off-target editing at all eight sites by an average of 2.3-, 7.4-, 13-, 22- and 37-fold compared to v7, while supporting on-target editing efficiencies of 126%, 98%, 78%, 66%, and 48% that of v7, respectively. Interestingly, at several sites the HS variants not only reduced off-target editing as expected but also increased on-target editing relative to v7. These results confirm that the HS variants identified support improved ZF-DdCBE specificity profiles across a variety of different mitochondrial sites, and across canonical or N-terminal-DddA ZF-DdCBE architectures. In particular, v8^{HS1} showed generally superior performance relative to v7 (an average 2.3-fold reduction in off-target editing with little or no reduction in on-target editing across all eight sites tested).

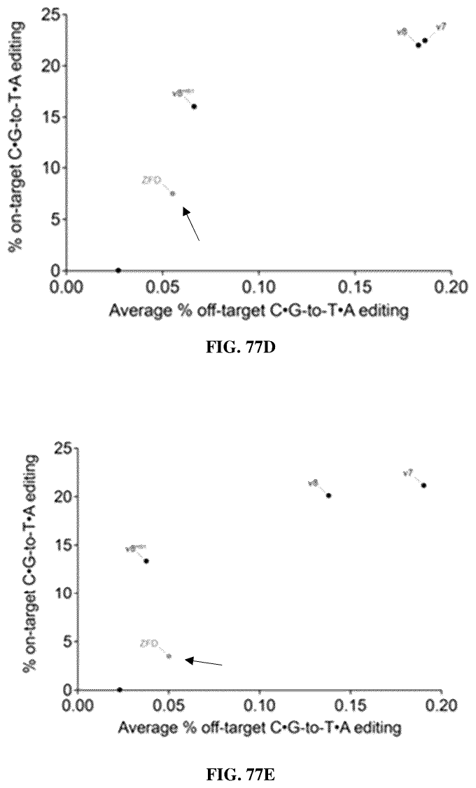

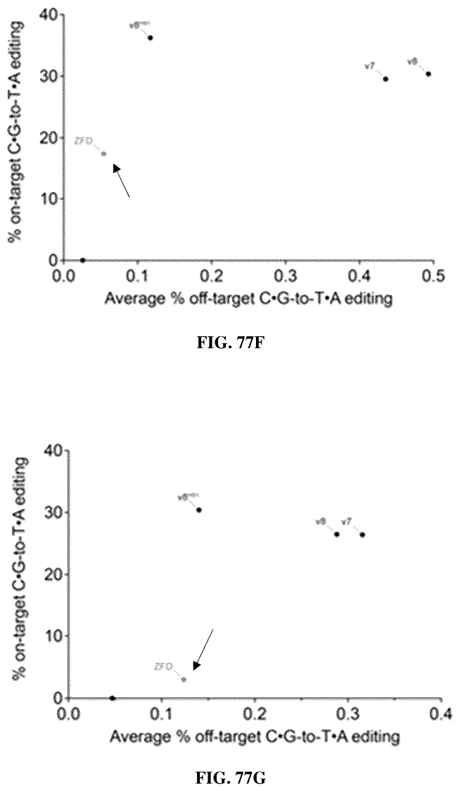

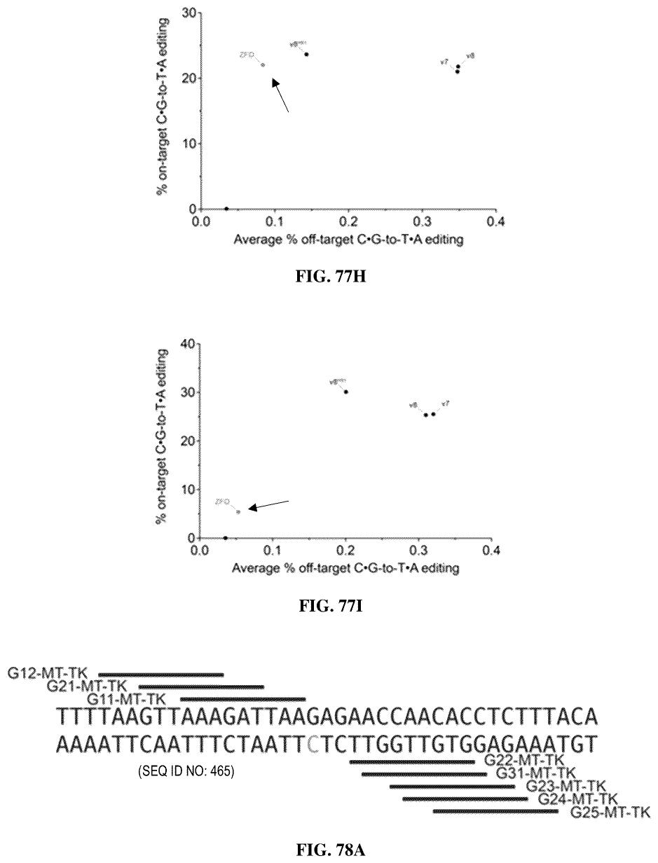

Lastly, the v8^{HS1} variant was used in nine ZF-DdCBE pairs derived from mtDNA-targeting ZFD pairs²⁵. Averaged across the nine pairs tested, v8^{HS1} variants reduced average off-target editing by 4.1-fold while retaining 90% on-target editing efficiency relative to v7 ZF-DdCBEs (FIGS. 77A-77I). Moreover, v8^{HS1} ZF-DdCBEs supported an average 3.1-fold higher on-target editing compared to ZFDs, concomitant with a 2.6-fold increase in average off-target editing. Collectively, these results demonstrate that strategies to minimize off-target editing caused by spontaneous split DddA reassembly can be integrated to engineer high-specificity ZF-DdCBE variants with minimal off-target editing and efficient on-target editing.

Installing Disease-Associated Edits in mtDNA in Cells In Vitro

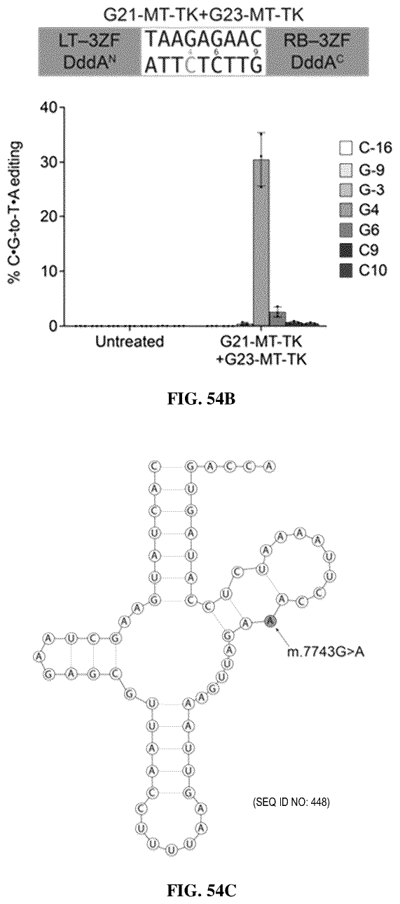

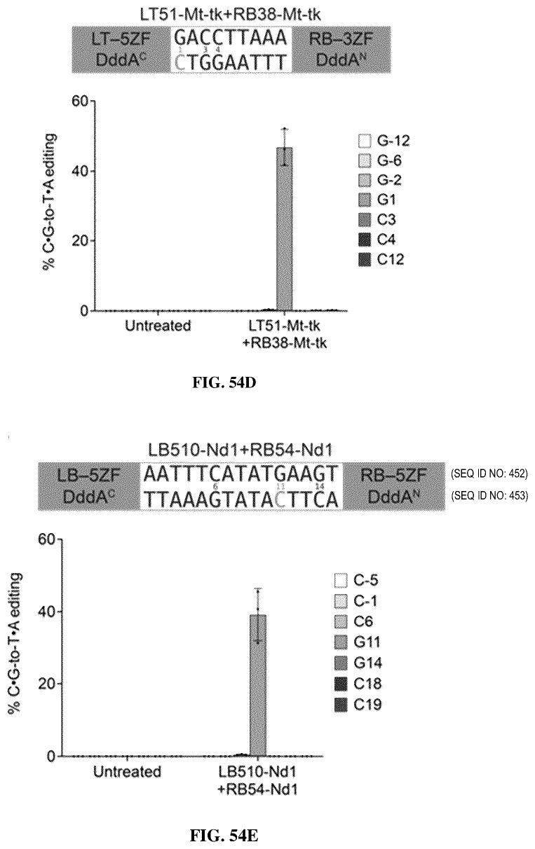

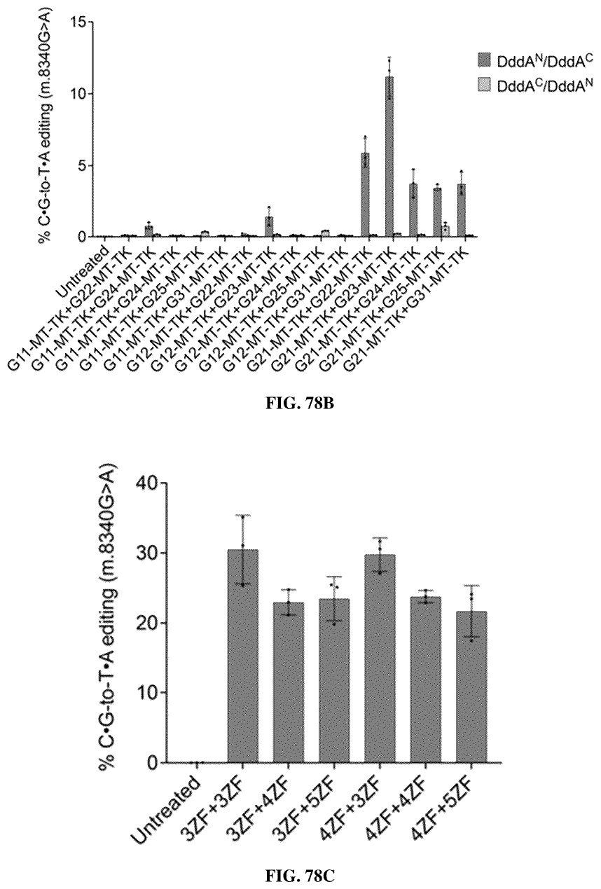

To demonstrate the utility of ZF-DdCBEs to install disease-associated mutations, ZF-DdCBEs were designed to install the m.8340G>A mutation within MT-TK in HEK293T cells. This mutation is associated with mitochondrial myopathy and retinopathy, creating a mismatch in the T-arm of mt-tRNA^{Lys} that impairs mitochondrial translation⁴¹⁻⁴⁴ (FIG. 54A). A panel of three left 3ZF ZF-DdCBEs with five right 3ZF ZF-DdCBEs was tested in both deaminase orientations (DddA^{N}+DddA^{C} and DddA^{C}+DddA^{N}), forming a total of 30 different combinations in v7 architecture (FIG. 78A). The top initial hit was able to install the m.8340G>A edit with an efficiency of 11% (FIG. 78B). For this best-performing ZF-DdCBE combination, extending each 3ZF to 4ZF or 5ZF was tested, but no improvement in on-target editing was observed (FIG. 78C). By testing alternative ZF scaffolds, v7^{AGKS} architecture was found to improve editing results, and this optimized ZF-DdCBE pair installed the m.8340G>A mutation with an efficiency of 31% (FIG. 54B). No substantial bystander editing was observed in the spacing region aside from 2.6% editing at position m.8342, which would create an additional mismatch in the mt-tRNA^{Lys} T-arm and be expected to further magnify the disease phenotype. These results show that ZF-DdCBEs can install targeted disease-associated mutations in human cells with high efficiency and specificity, creating model cell lines for the study of human mitochondrial genetic diseases.

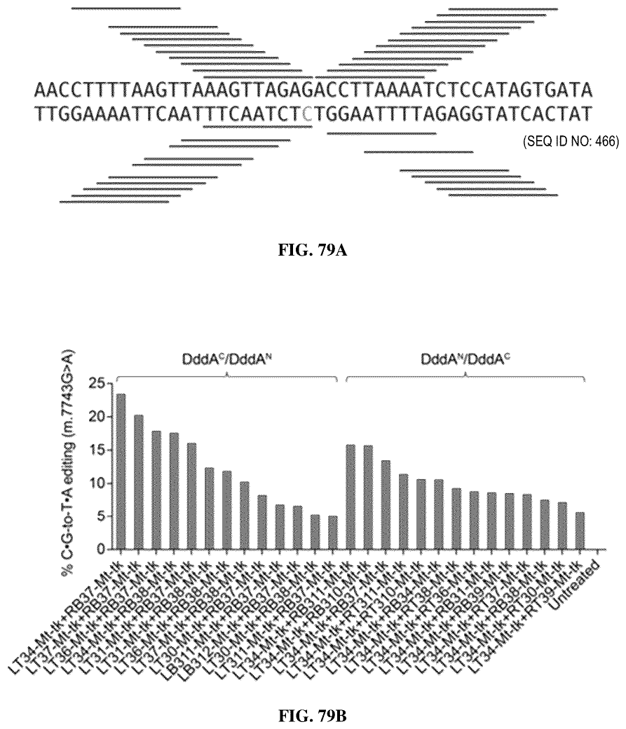

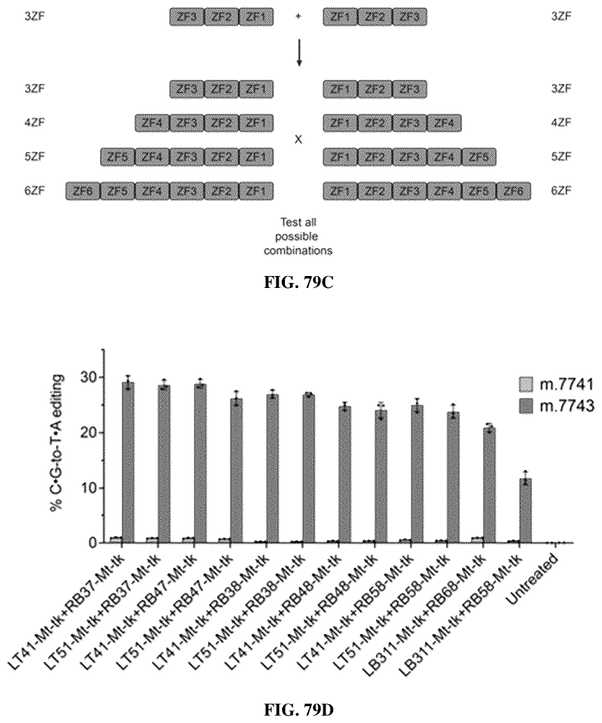

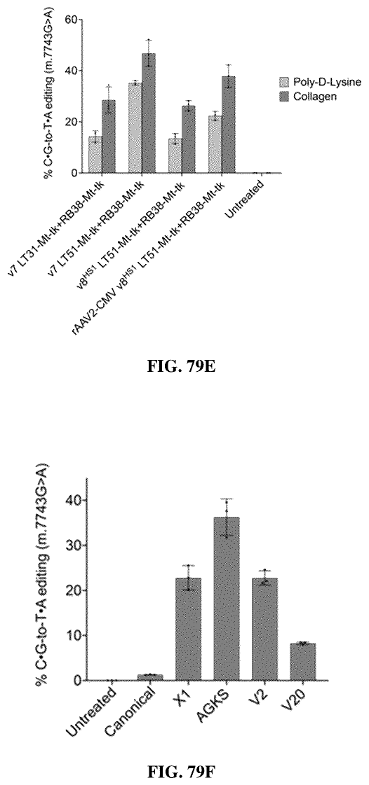

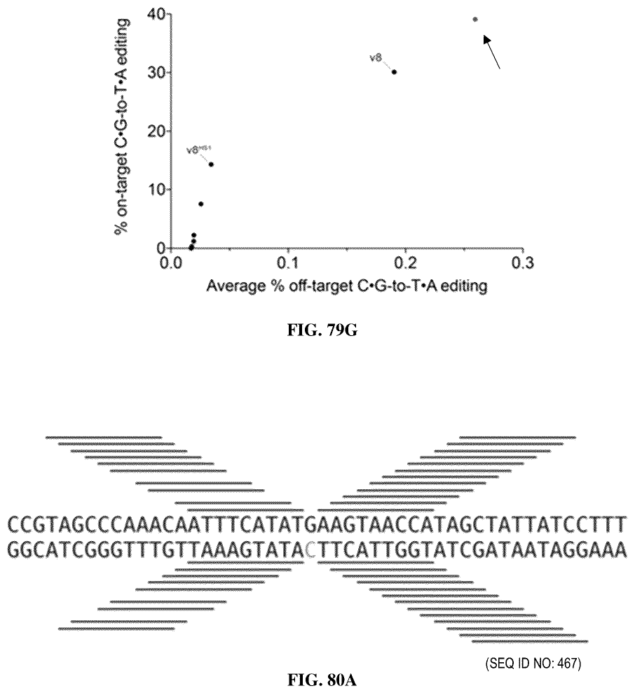

Next, it was investigated whether ZF-DdCBEs could be used in other mammalian cell lines to create biological models of human genetic diseases. Towards creating a mouse model of the human m.8340G>A genetic disease, installing the m.7743G>A mutation in mouse C2C12 cells was explored (FIG. 54C). Because human MT-TK and mouse Mt-tk genes share only 60% sequence identity, this lack of sequence conservation necessitated designing and optimizing a new set of ZF-DdCBE pairs in the murine context. A panel of 20 left 3ZF ZF-DdCBEs with 19 right 3ZF ZF-DdCBEs were tested in both deaminase orientations, forming 760 pairwise combinations in v7^{AGKS} architecture (FIG. 79A). 27 ZF-DdCBE pairs able to install the desired edit with efficiencies ranging from 5% to 23% were identified (FIG. 79B). These pairs were optimized by extending each 3ZF to 4ZF, 5ZF, or 6ZF where possible, and alternative ZF scaffolds were tested. Initially, 27 ZF-DdCBE pairs were identified as being able to install the desired edit in mouse C2C12 cells with efficiencies ranging from 5% to 23% (FIG. 79B). To assess whether ZF extension could improve editing performance, for these 27 pairs each 3ZF to 4ZF, 5ZF, or 6ZF was extended where possible, and the resulting ZF-DdCBE combinations were tested (FIG. 79C). Additional ZF repeats were added to the ZF arrays extending away from the spacing region in order to maintain a fixed deaminase positioning. From the 12 best-performing ZF-DdCBE combinations, a pair (LT51-Mt-tk+RB38-Mt-tk) that showed a good balance between high on-target activity and low bystander or off-target editing was selected (FIG. 79D). This final 3ZF+5ZF v7^{AGKS} ZF-DdCBE pair exhibited a 2.5-fold improvement relative to its corresponding 3ZF+3ZF pair, installing the m.7743G>A mutation at an efficiency of 35% and with excellent specificity (FIG. 79E). Alternative ZF scaffolds were tested, and it was confirmed that v7^{AGKS} architecture supported the highest on-target editing efficiency for this ZF-DdCBE pair (FIG. 79F). It was also discovered that editing efficiency could be increased to 47% by plating C2C12 cells on collagen-coated plates instead of poly-D-lysine-coated plates (FIG. 79E).

An optimized ZF-DdCBE pair (LT51-Mt-tk+RB38-Mt-tk) was selected that offered a good balance between high on-target activity and low bystander or off-target editing. This final 5ZF+3ZF v7^{AGKS} ZF-DdCBE pair exhibited a 1.6-fold improvement relative to its corresponding 3ZF+3ZF pair, installing the m.7743G>A mutation at an efficiency of 47% and with excellent specificity (FIG. 54D). v8^{HS} variants of this ZF-DdCBE pair were confirmed to decrease off-target editing by 14-fold and 10-fold, while retaining 37% and 48% on-target editing compared to v7 and v8, respectively (FIG. 79G). Collectively, these results show that ZF-DdCBEs can be used to create biological models of human genetic disease and install targeted disease-associated mutations in different cell lines from different organisms with good efficiency and specificity.

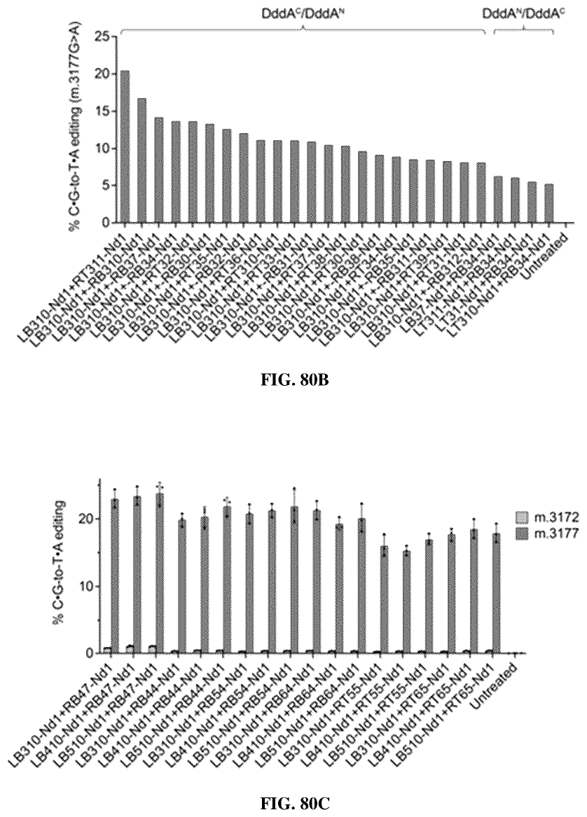

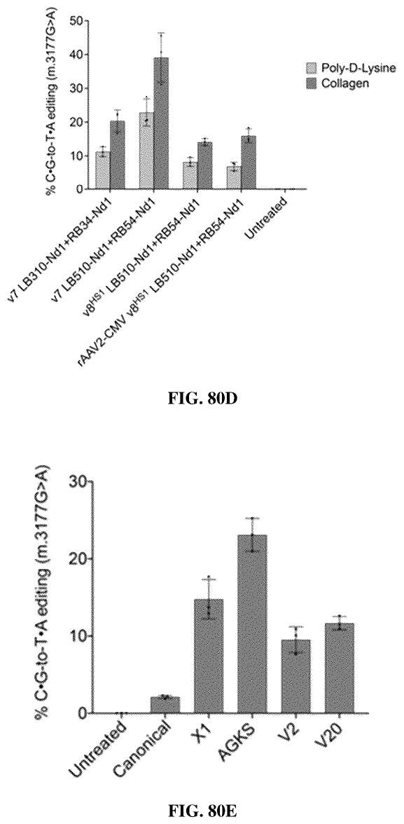

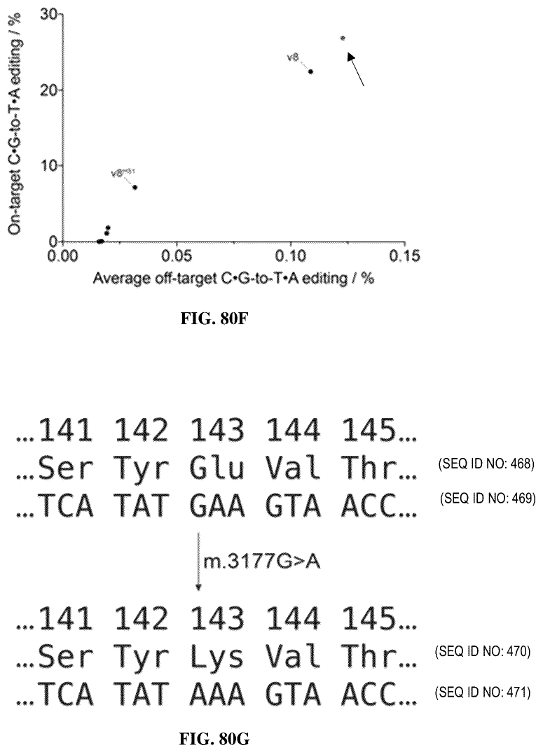

As a second demonstration of using ZF-DdCBEs to create biological models of human genetic diseases, the m.3177G>A mutation was installed in mouse C2C12 cells, creating a missense E143K mutation in the mitochondrial Nd1 gene associated with Leber's hereditary optic neuropathy (LHON)⁴⁵⁻⁴⁶ (FIG. 80G). A panel of 19 left 3ZF ZF-DdCBEs with 25 right 3ZF ZF-DdCBEs were tested in both deaminase orientations, forming 950 pairwise combinations in v7^{AGKS} architecture (FIG. 80A). 26 ZF-DdCBE pairs able to install the desired edit with efficiencies ranging from 5% to 20% were identified (FIG. 80B). These pairs were optimized by extending each 3ZF to 4ZF, 5ZF, or 6ZF where possible, and alternative ZF scaffolds were tested. 26 ZF-DdCBE pairs were identified as being able to install the desired edit with efficiencies ranging from 5% to 20% (FIG. 80B). To assess whether ZF extension could improve editing performance, for 34 pairs each 3ZF to 4ZF, 5ZF, or 6ZF were extended where possible, and the resulting ZF-DdCBE combinations were tested (FIG. 79C). From the 18 best-performing ZF-DdCBE combinations, a pair (LB510-Nd1/RB54-Nd1) was selected that showed a good balance between high on-target activity and low bystander or off-target editing (FIG. 80C). This final 5ZF+5ZF v7^{AGKS} ZF-DdCBE pair exhibited a 2.0-fold improvement relative to the unoptimized 3ZF+3ZF pair, installing the m.3177G>A mutation at an efficiency of 23% and with excellent specificity (FIG. 80D). Alternative ZF scaffolds were tested, and it was confirmed that v7^{AGKS} architecture supported the highest on-target editing efficiency for this ZF-DdCBE pair (FIG. 80E). It was also discovered that editing efficiency could be increased to 39% by plating C2C12 cells on collagen-coated plates instead of poly-D-lysine-coated plates (FIG. 80D).

A pair (LB510-Nd1+RB54-Nd1) was selected that showed a good balance between high on-target activity and low bystander or off-target editing. This final 5ZF+5ZF v7^{AGKS} ZF-DdCBE pair exhibited a 1.9-fold improvement relative to its corresponding 3ZF+3ZF pair, installing the m.3177G>A mutation at an efficiency of 39% and with excellent specificity (FIG. 54E). To minimize off-target editing, v8^{H}S variants of this ZF-DdCBE pair were tested, and v8^{HS1} was observed to reduce average off-target editing by 6.8-fold and 5.9-fold, while retaining 27% and 32% on-target editing compared to v7 and v8 respectively (FIG. 80F). Collectively, these results establish ZF-DdCBEs as a useful tool for the creation of biological models of human genetic diseases through the efficient and precise installation of targeted disease-associated mutations.

### ZF-DdCBEs Enable Base Editing of Nuclear DNA

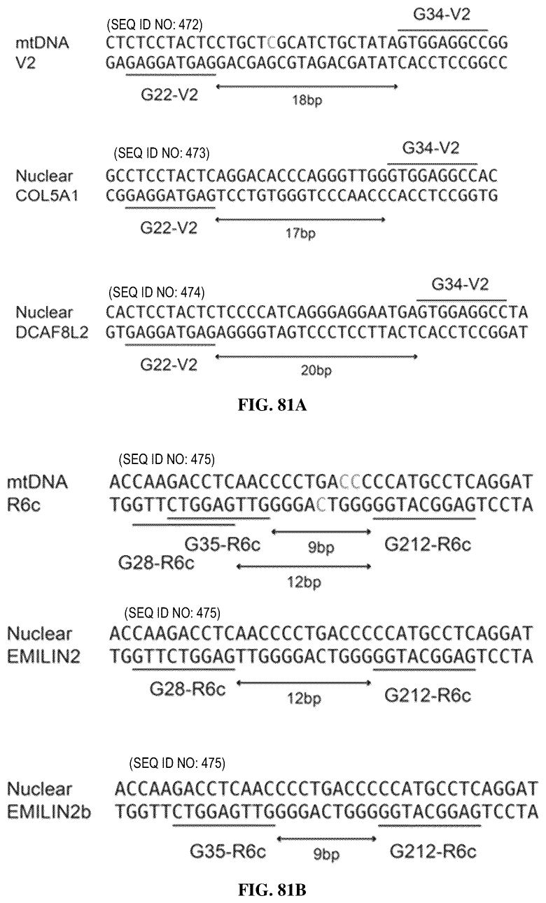

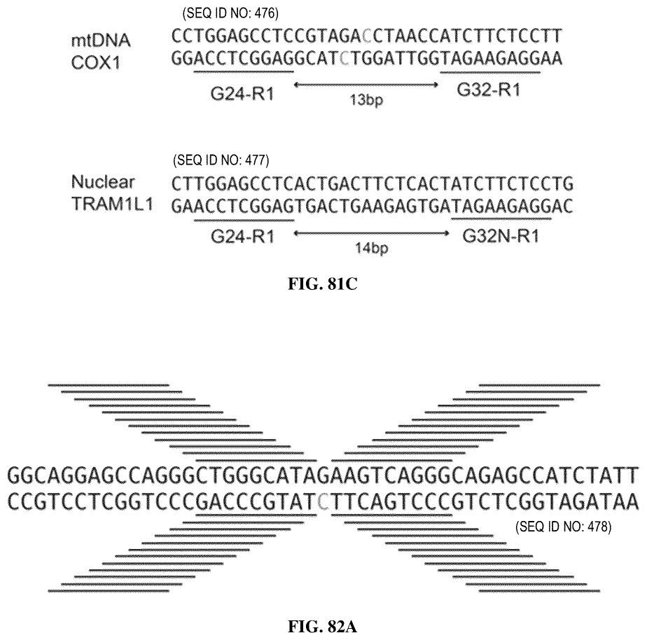

To test whether ZF-DdCBEs are capable of mediating targeted C•G-to-T•A conversion in nuclear DNA, validated mitochondrial ZF-DdCBEs were converted into nuclear ZF-DdCBEs. Sites in mtDNA that were edited by optimized 3ZF+3ZF ZF-DdCBEs with high efficiency in HEK293T cells were selected, and the human nuclear genome was searched for corresponding sites with high sequence similarity. Nuclear sites were identified that shared conserved ZF binding sites with no mismatches, were separated by a spacing region within ±2 bp in length compared to the mtDNA target's spacing region, and contained TC dinucleotides at similar positions within the spacing region compared to the target nucleotide(s) efficiently edited in mtDNA (FIGS. 81A-81C).

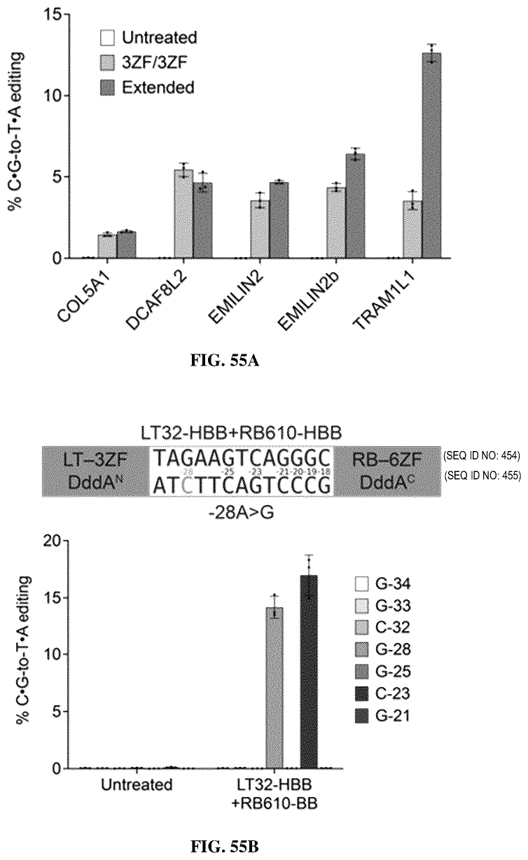

To create nuclear-targeted ZF-DdCBEs, the mitochondria-targeted v7 ZF-DdCBE architecture was adapted by replacing the N-terminal MTS and NES sequences with four NLS sequences (two SV40 bipartite NLS and two cMyc NLS), and the additional copy of mitochondrially targeted UGI expressed in trans was removed. Four nuclear-targeted 3ZF+3ZF ZF-DdCBE pairs were tested at five sites in nuclear DNA, and editing efficiencies in HEK293T cells ranging from 1-5% were observed across the five sites tested. Extending each 3ZF array to 4ZF, 5ZF, or 6ZF was tested, and improvements in editing efficiency for four of the five pairs tested were observed, with on-target editing efficiencies ranging from 2-13% (FIG. 55A). These results establish that ZF-DdCBEs support all-protein nuclear base editing, even when designing ZFs using the simple modular assembly approach.

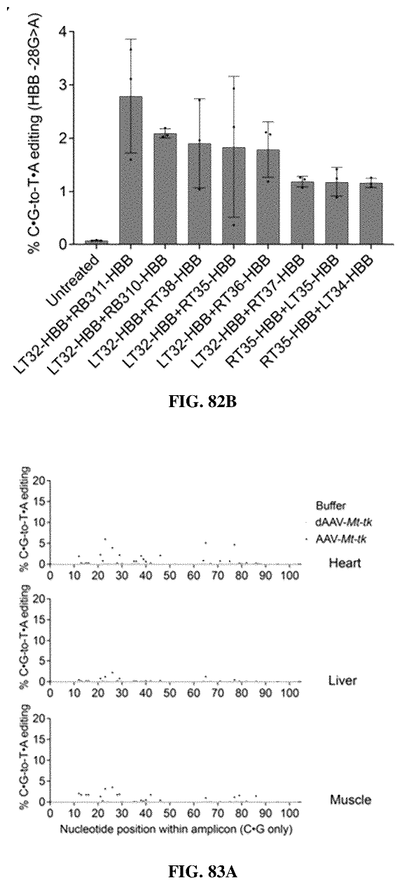

To demonstrate the ability of ZF-DdCBEs to correct disease-causing mutations in nuclear DNA, the −28(A>G) mutation in the promoter region of the human HBB gene that causes 0-thalassemia⁴⁷ was corrected. A panel of 24 left 3ZF ZF-DdCBEs with 24 right 3ZF ZF-DdCBEs was tested in both deaminase orientations (FIG. 82A) in HEK293T-HBB cells that have a lentivirus-integrated 200-bp fragment of the mutated HBB promoter sequence locus⁴⁸. Eight 3ZF+3ZF ZF-DdCBE pairs that performed the desired edit with 1-3% efficiencies were identified (FIG. 82B). These pairs were optimized by extending each 3ZF to 4ZF, 5ZF, or 6ZF, and the most efficient ZF-DdCBE pair installed the desired edit with an editing efficiency of 14%, a 6.8-fold improvement relative to the unoptimized 3ZF+3ZF pair, together with 17% bystander editing corresponding to −23C>T (FIG. 55B). This bystander mutation lies downstream of the HBB promoter's non-canonical TATA-box (CATAAA) bound by transcription factor TFIID⁴⁹, and is not known to be associated with any globinopathy⁵. Collectively, these results demonstrate that ZF-DdCBEs can correct pathogenic mutations in nuclear DNA, albeit less efficiently than canonical nuclease base editors.

In Vivo Base Editing of Pathogenic Target Sites in mtDNA

An important advantage of the reduced size of ZF-DdCBEs compared to TALE-based DdCBEs is their ability to be packaged into a single AAV capsid for in vivo delivery. To validate that ZF-DdCBE pairs could be expressed as a single operon, rAAV2-CMV expression vectors⁵¹ encoding v8^{HS1} ZF-DdCBE pairs designed to install either the murine m.7743G>A or m.3177G>A mutation were created and expressed under a single CMV promoter using a self-cleaving P2A peptide between each ZF-DdCBE half. It was verified that these constructs retained editing activity in C2C12 cells, installing either m.7743G>A or m.3177G>A with an editing efficiency of 38% and 16%, respectively (FIG. 79E, and FIG. 80D). To facilitate bacterial cloning, a cassette for constitutive bacterial expression DddI, the natural protein inhibitor of DddA, was installed into the vector backbone at a location that would not be packaged into AAV genomes. These results demonstrate that ZF-DdCBE pairs can mediate good editing efficiency when expressed as a single gene (2.4 and 2.5 kb in length, respectively) that is much smaller in size than the AAV packaging limit of ˜4.7 kb, suggesting that ZF-DdCBEs might be suitable for single AAV-mediated delivery (FIG. 57).

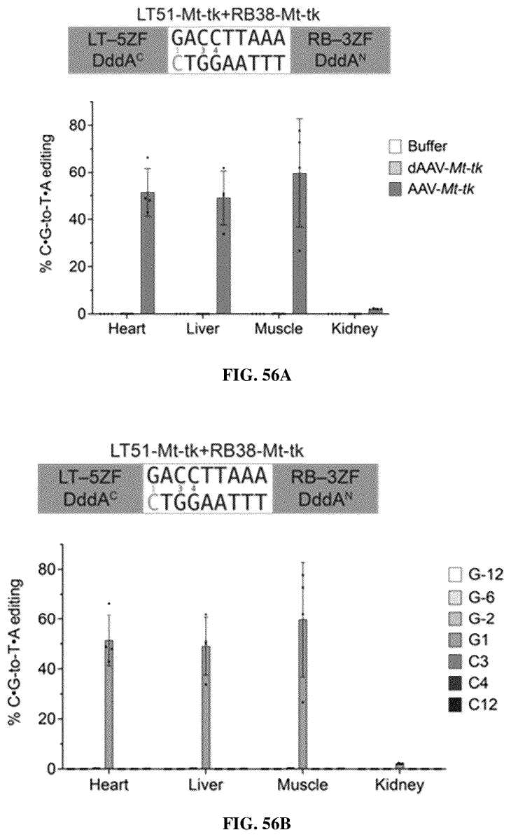

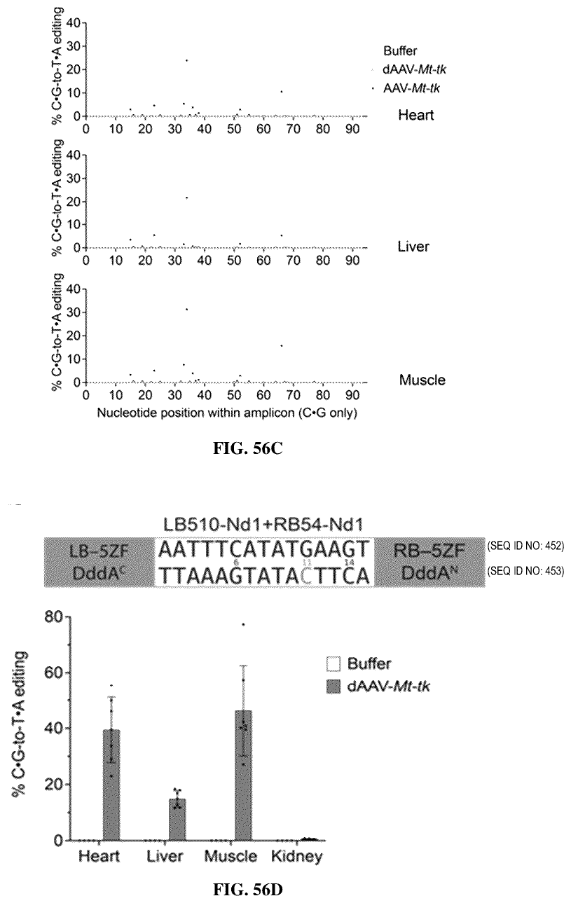

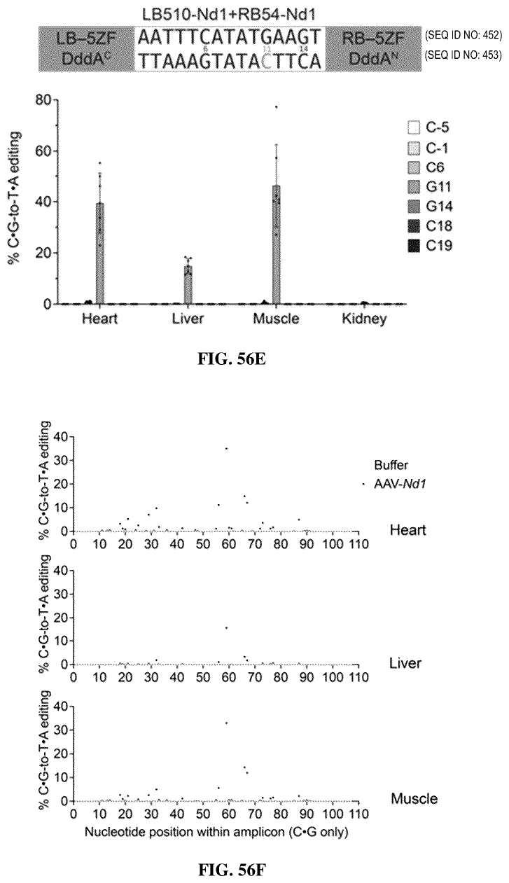

To investigate the performance of ZF-DdCBEs in vivo, after recombinant AAV2/9 production 7.5×10¹¹ viral genomes (AAV-Mt-tk or AAV-Nd1, encoding v8^{HS1} ZF-DdCBE pairs installing m.7743G>A or m.3177G>A, respectively) were delivered into newborn P1 mice by intravenous injection, and tissue samples were harvested for DNA sequencing after 14-30 days. Robust editing was observed in the heart, liver, quadriceps skeletal muscle and kidney, with average on-target editing activities of 51±10%, 49±12%, 60±23%, and 2.1±0.2% for AAV-Mt-tk and 39±12%, 15±3%, 46±16%, and 0.5±0.2% for AAV-Nd1, respectively, and with editing profiles similar to those observed in C2C12 cells in vitro (FIGS. 56A-56B, FIGS. 56D-56E). As a negative control, editing following AAV delivery encoding the Mt-tk-targeting ZF-DdCBE pair containing the DddA-inactivating E1347A mutation was not observed (dAAV-Mt-tk) (FIG. 56A).

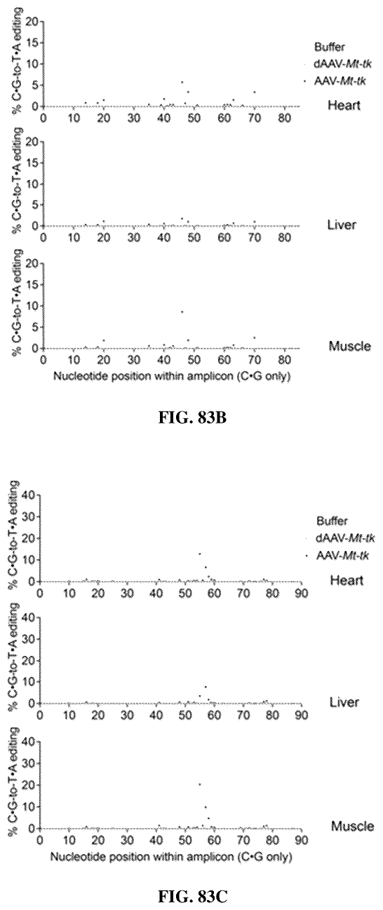

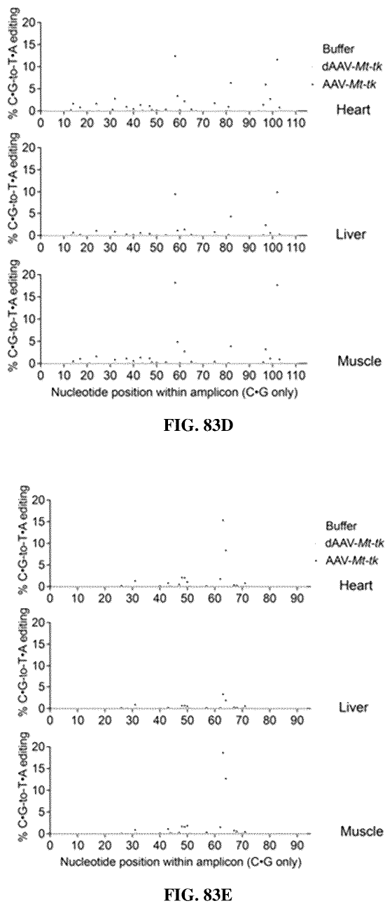

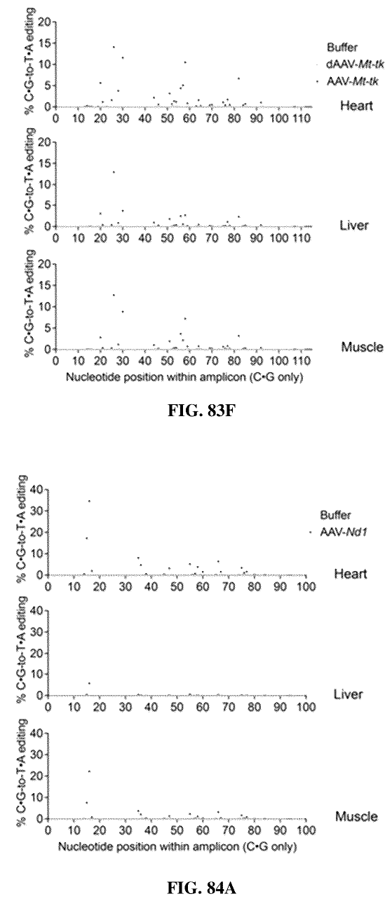

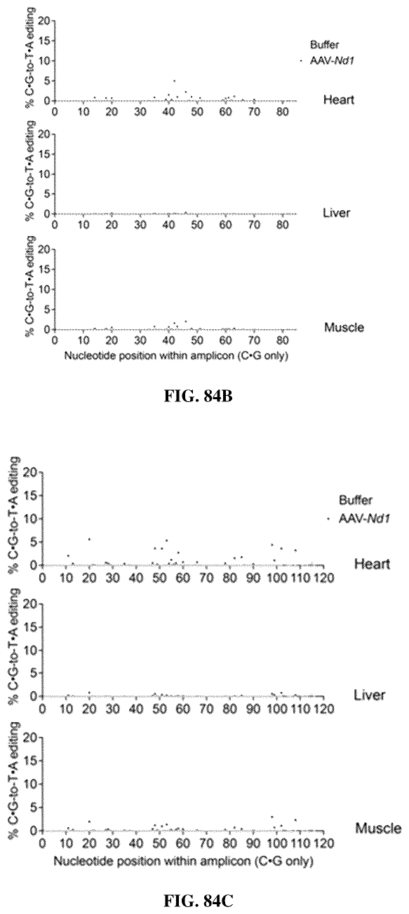

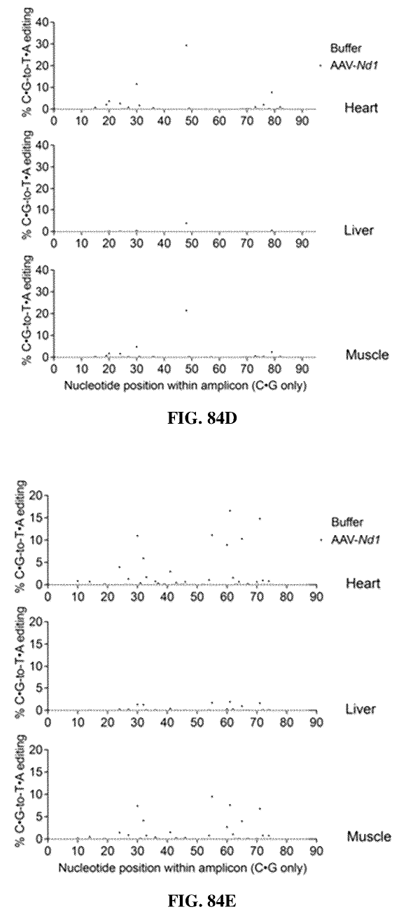

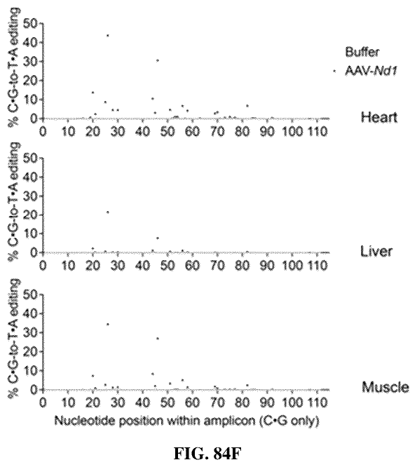

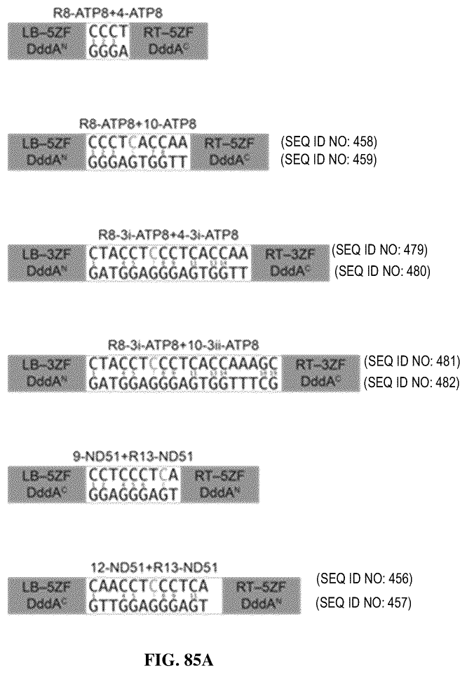

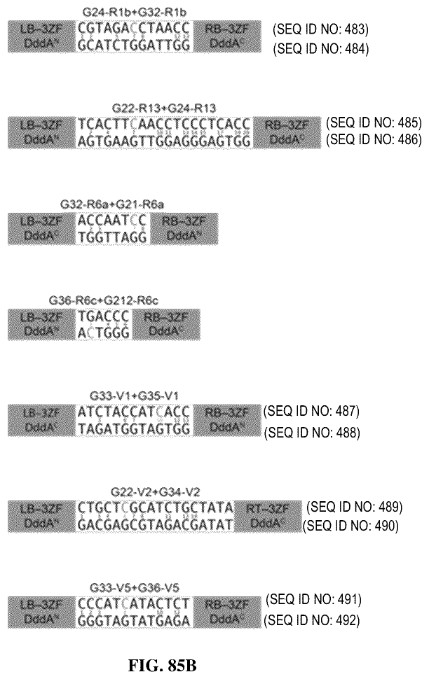

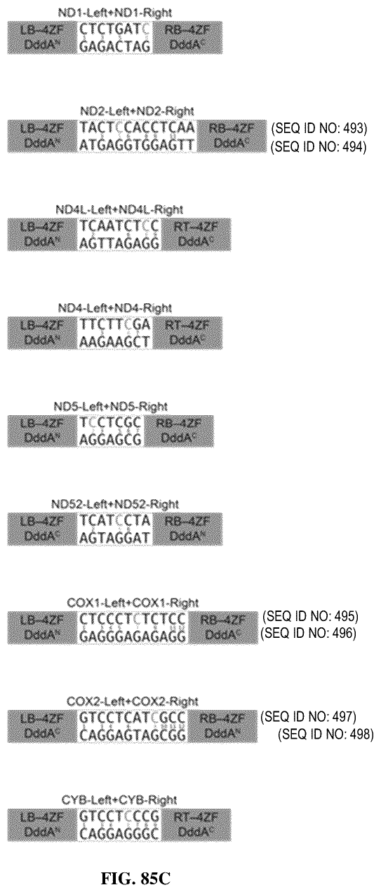

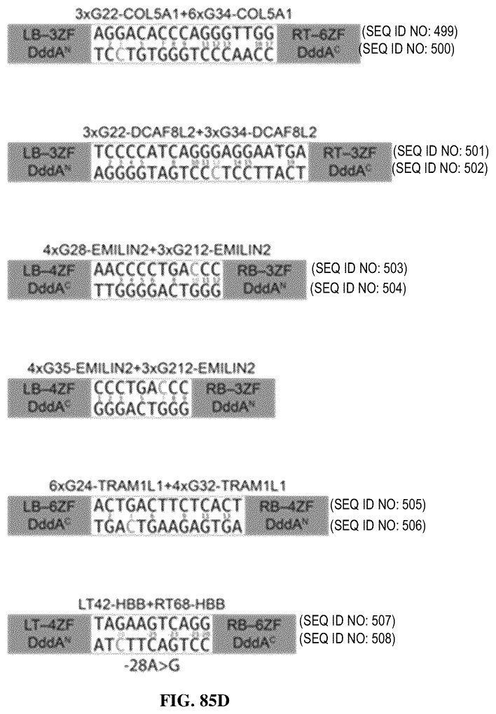

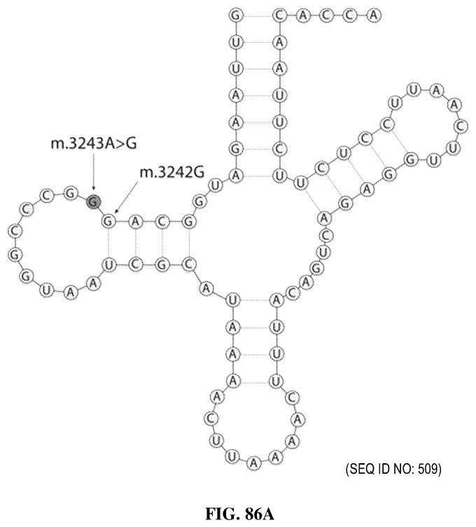

To assess in vivo off-target editing, targeted amplicon sequencing was performed at predicted ZF off-target sites. For mice treated with AAV-Nd1, seven amplicons that contained the top eight off-target ZF binding sites in mtDNA as predicted by sequence similarity (four off-target sites for the left 5ZF array and four off-target sites for the right 5ZF array, each containing three nucleotide mismatches) were sequenced. For mice treated with AAV-Mt-tk, seven amplicons that contained 14 off-target ZF binding sites in mtDNA as predicted by sequence similarity (eight off-target sites for the left 5ZF array containing three or four nucleotide mismatches and six off-target sites for the right 3ZF array containing three nucleotide mismatches) were sequenced. Off-target editing was observed at C•G base pairs scattered across each predicted off-target site, typically with efficiencies≥10-fold lower than that of the on-target edit in the same tissues, although some C•G base pairs flanking the predicted off-target ZF binding sites were edited more efficiently (FIG. 56C, FIG. 56F, FIGS. 83A-83F, and FIGS. 84A-84F). The in vivo durability of AAV, which can support ZF-DdCBE expression throughout the 14-30 days of the experiment⁵², likely resulted in the accumulation of these off-target edits. The use of transient mRNA or RNP delivery methods instead of AAV, or recently developed methods to limit the duration of AAV expression⁵³⁻⁵⁵, should reduce off-target editing in vivo. These results collectively demonstrate that ZF-DdCBEs enable efficient in vivo editing of mtDNA via single-AAV delivery and can be used in mice to install disease-associated point mutations in a variety of tissues.

### Discussion

Optimized ZF-DdCBEs capable of base editing both mitochondrial and nuclear DNA that are substantially smaller and less repetitive than TALE-containing DdCBEs were created. This size reduction was demonstrated to facilitate packaging within a single AAV9 capsid for efficient in vivo base editing of mtDNA, in contrast with dual-AAV approaches used for the in vivo delivery of TALE-based DdCBEs⁵⁶. Additionally, approaches to minimize off-target editing by reducing spontaneous split DddA reassembly were identified. For maximum on-target editing efficiency, starting with v7 architecture using ZF scaffold X1 is recommended. After identifying high-performing ZF-DdCBE pairs, testing alternative ZF scaffolds (AGKS, V2, V20) to determine whether these lead to improvements is recommended, and incorporating variants HS1-HS5 when minimizing off-target editing is critical. Delivery of ZF-DdCBEs in mRNA or protein form should further reduce off-target editing^{25, 57-59}.

Since shorter ZF arrays are less expensive to construct, starting with pairs of 3ZF+3ZF ZF-DdCBEs, which can support efficient editing in mitochondria, is suggested before testing longer ZF arrays to maximize editing efficiency. For nuclear targets it may be beneficial to start with longer ZF arrays. Testing a panel of ZF-DdCBEs for each user-defined target to identify efficient ZF-DdCBE pairs is recommended. Although straightforward, the modular assembly approach for constructing ZFs has a higher failure rate and can yield less potent DNA-binding ZF arrays than methods that use in vivo selection³¹. More sophisticated approaches to ZF design, such as iterated library screening and selection that account for context-dependent effects^{60, 61}, should result in ZF-DdCBEs with more potent target binding activity and specificity.

While all base editors must place the target nucleotide(s) within an editing window, unlike TALE- or CRISPR-containing CBEs, it was demonstrated that using both canonical and N-terminal architectures allows ZF-DdCBEs to be designed to bind to either the same or opposite DNA strands around the target nucleotide(s). Several of the active ZF-DdCBE pairs described herein support efficient editing with much smaller spacing regions than TALE-DdCBEs, thus reducing the number of non-target cytosines within the editing window and minimizing bystander editing. These features of ZF-DdCBEs offer more flexibility when designing ZF arrays than TALE-DdCBEs.

### Methods

### General Methods and Molecular Cloning

All plasmids were constructed by Gibson assembly using NEBuilder HiFi DNA Assembly Master Mix (New England Biolabs) or synthesized and cloned by Twist Biosciences and transformed into MachOne T1^{R} chemically competent E. coli cells (Thermo Fisher Scientific). DNA primers were ordered from Integrated DNA Technologies, and PCR was performed using PrimeSTAR GXL DNA Polymerase (Takara Bio). Synthetic DNA was ordered as eblock or gblock fragments from Integrated DNA Technologies (IDT). Codon optimization was performed either manually or using IDT's Codon Optimization Tool. Plasmid DNA was amplified by rolling circle amplification using a TempliPhi Amplification Kit (Cytiva) prior to Sanger sequencing for sequence confirmation. Plasmids were purified using QIAprep Spin Miniprep kits (Qiagen) and quantified using a NanoDrop One spectrophotometer (Thermo Fisher Scientific).

### General Mammalian Cell Culture Conditions

HEK293T (CRL-3216) and C2C12 (CRL-1772) cells were purchased from American Type Culture Collection (ATCC) and cultured and passaged in DMEM supplemented with GlutaMAX (Thermo Fisher Scientific) and 10% (v/v) FBS (Gibco, qualified). Cells were incubated, maintained, and cultured at 37° C. with 5% CO₂. Cell lines were authenticated by their respective suppliers and tested negative for mycoplasma.

### Tissue Culture Transfection and Genomic DNA Extraction

Cells were seeded on 48-well poly-D-lysine-coated plates (Corning), or 48-well collagen-coated plates (Corning) where specified, in a volume of 250 μl per well at a density of 6×10⁴ cells/ml for human cells or a density of 2×10⁴ cells/ml for C2C12 cells. 24 hours after seeding, cells were transfected with a total of 25 μl lipofection mix in Opti-MEM (Thermo Fisher Scientific) containing 1 μg plasmid DNA (500 ng each ZF-DdCBE) and 1.5 μl Lipofectamine 2000 (Thermo Fisher Scientific) at approximately 40% confluency. Cells were harvested 3 days after transfection for genomic DNA (gDNA) extraction. Medium was removed, and cells were washed once with PBS (Thermo Fisher Scientific). Cells were lysed by the addition of 80 μl freshly prepared lysis buffer (10 mM Tris-HCl (pH 8.0), 0.05% SDS, and 25 μg/ml proteinase K (Thermo Fisher Scientific)) and incubated at 37° C. for 1 hour before proteinase K was inactivated at 80° C. for 30 minutes. Genomic DNA was stored at −20° C. until used.

### High-Throughput DNA Sequencing of Genomic DNA Samples

Genomic sites of interest were amplified from genomic DNA samples and sequenced on an Illumina MiSeq. Amplification primers containing Illumina forward and reverse adapters (See Tables 1-30) were used for a first round of PCR (PCR1) to amplify the genomic region of interest. 25 μl PCR1 reactions were performed using Phusion Hot Start II High-Fidelity DNA Polymerase (Thermo Fisher Scientific) with 2 μl genomic DNA extract and supplemented with 0.5×SYBR Green I (Thermo Fisher Scientific), and monitored by quantitative PCR (CFX96, Bio-Rad). The PCR1 protocol was 98° C. for 120 seconds, then 30 cycles of 98° C. for 10 seconds, 62° C. for 20 seconds, and 72° C. for 30 seconds, followed by a final 72° C. extension for 120 seconds. Unique Illumina barcodes were added to each sample in a secondary PCR (PCR2). 25 μl PCR2 reactions were performed using Phusion Hot Start II High-Fidelity DNA Polymerase (Thermo Fisher Scientific) with 2 μl unpurified PCR1 product. The PCR2 protocol was 98° C. for 120 seconds, then 10 cycles of 98° C. for 10 seconds, 61° C. for 20 seconds, and 72° C. for 30 seconds and followed by a final 72° C. extension for 120 seconds. PCR2 products were pooled by common amplicons and purified by gel electrophoresis with a 2% agarose gel using a QIAquick Gel Extraction kit (Qiagen). DNA was quantified using a Qubit dsDNA High Sensitivity Assay kit (Thermo Fisher Scientific) and sequenced using an Illumina MiSeq with single-end reads. Sequencing results were computed with a minimum sequencing depth of approximately 10,000 reads per sample.

### Analysis of High-Throughput Sequencing Data for Targeted Amplicon Sequencing

Sequencing reads were demultiplexed using MiSeq Reporter (Illumina) and analyzed by amplicon using CRISPResso2 (version 2.1.3)⁶² using default parameters. Tables 1-30 contain a list of amplicon sequences used for alignment. A cleavage offset of ˜8 was used, and a 16 bp spacing region between ZF-DdCBEs was supplied in place of the input sgRNA sequence. A 10 bp window was used to quantify indels centered around the middle of the spacing region between ZF-DdCBEs. The output file Nucleotide_percentage_summary.txt was imported into Microsoft Excel (Microsoft) for quantification of editing frequencies. Reads containing indels within the 10-bp window are excluded for calculation of editing frequencies. The output file CRISPRessoBatch_quantification_of_editing_frequency.txt was imported into Microsoft Excel (Microsoft) for calculation of indel frequencies. Indel frequencies were computed by dividing the number of aligned reads containing insertions or deletions by the total number of aligned reads. Average off-target editing efficiencies were calculated by averaging the C•G-to-T•A editing efficiency across all C•G base pairs within the amplicon. For amplicons containing the spacing region targeted by a ZF-DdCBE pair, nucleotides±10 bp upstream and downstream of the nucleotide with the highest on-target C•G-to-T•A editing efficiency were excluded from the analysis. All graphs were plotted using Prism 8 (GraphPad).

### Bioinformatic Searches

ScanProsite⁶³ was used to search the human proteome for ZF-containing sequences, submitting the motif x(6)-C-x(2)-C-x(12)-H-x(3)-H-x(5) as a query to scan against the UniProtKB protein sequence datable, using Homo sapiens as a taxonomical filter. Sequence logos were generated using WebLogo 3⁶⁴, available online at weblogo.threeplusone.com/create.cgi. Nuclear sites with high sequence similarity to validated mitochondrial ZF-DdCBE targets were identified using ZFN-Site⁶⁵, available online at ccg.epfl.ch/tagger/targetsearch.html. Queries used settings of zero mismatches per half-site and disallowing left and right protein homo-dimerization.

### Viral Vector Production and In Vivo Animal Experiments

ZF-DdCBE-expressing rAAV2-CMV vectors were used to generate recombinant AAV2/9 viral particles at the University of North Carolina at Chapel Hill Vector Core. Mice in a C57BL/6J background were obtained from Charles River Laboratories. The animals were maintained in a temperature- and humidity-controlled animal care facility with a 12 hour light/12 hour dark cycle and free access to water and food and sacrificed by cervical dislocation. Newborn mice (postnatal day 1—males and females) were injected with 7.5×10¹¹ AAV particles via the temporal vein using a 30 G, 30°-beveled needle syringe. Control mice were injected with similar volumes of vehicle buffer (1×PBS, 230 mM NaCl and 5% (w/v) D-sorbitol). Samples from the heart, quadriceps, liver, and kidney were snap-frozen in liquid nitrogen at sacrifice and stored at −80° C. until used. Genomic DNA from mouse tissue samples was extracted using a DNeasy Blood & Tissue kit (Qiagen).

TABLE 1
Tables for Example 3
Mitochondrial ZF-DdCBEs, canonical architecture

| | Archi- | | ZF | DddA | Composition (N- |
|---|---|---|---|---|---|
| Name | tecture | Sequence | scaffold | split | to C-terminus) |
| R8-3i- | v1 | MLGFVGRVAAAPASGALRRLTPSASLPPAQL | Canonical | DddA^{N} | MTS_NES_2-aa |
| ATP8 | | LLRAAPTAVHPVRDYAAQTSVDEMTKKFGT | | | linker_ZF |
| | | LTIHDTEKAAMAERPFQCRICMRNFSTSGSLS | | | array_2-aa |
| | | RHIRTHTGEKPFACDICGRKFAQSGSLTRHTKI | | | linker_DddA^{N}_4- |
| | | HTGQKPFQCRICMRNFSRSDALSQHTKIHLRG | | | aa linker_UGI |
| | | SGSYALGPYQISAPQLPAYNGQTVGTFYYVN | | | |
| | | DAGGLESKVFSSGGPTPYPNYANAGHVEGQS | | | |
| | | ALFMRDNGISEGLVFHNNPEGTCGFCVNMTE | | | |
| | | TLLPENAKMTVVPPEGSGGSTNLSDIIEKETG | | | |
| | | KQLVIQESILMLPEEVEEVIGNKPESDILVHTA | | | |
| | | YDESTDENVMLLTSDAPEYKPWALVIQDSNG | | | |
| | | ENKIKML (SEQ ID NO: 347) | | | |
| R8-3i- | v2 | MLGFVGRVAAAPASGALRRLTPSASLPPAQL | Canonical | DddA^{N} | MTS_NES_2-aa |
| ATP8 | | LLRAAPTAVHPVRDYAAQTSVDEMTKKFGT | | | linker ZF |
| | | LTIHDTEKAAMAERPFQCRICMRNFSTSGSLS | | | array_13-aa |
| | | RHIRTHTGEKPFACDICGRKFAQSGSLTRHTKI | | | Gly/Ser-rich |
| | | HTGQKPFQCRICMRNFSRSDALSQHTKIHLRG | | | flexible |
| | | SGGGGSGGSGGSGSYALGPYQISAPQLPAYN | | | linker_DddA^{N}_4- |
| | | GQTVGTFYYVNDAGGLESKVFSSGGPTPYPN | | | aa linker_UGI |
| | | YANAGHVEGQSALFMRDNGISEGLVFHNNPE | | | |
| | | GTCGFCVNMTETLLPENAKMTVVPPEGSGGS | | | |
| | | TNLSDIIEKETGKQLVIQESILMLPEEVEEVIGN | | | |
| | | KPESDILVHTAYDESTDENVMLLTSDAPEYKP | | | |
| | | WALVIQDSNGENKIKML(SEQ ID NO: 348) | | | |
| R8-3i- | v3 | MLGFVGRVAAAPASGALRRLTPSASLPPAQL | Canonical | DddA^{N} | MTS_FLAG |
| ATP8 | | LLRAAPTAVHPVRDYAAQDYKDDDDKVDE | | | tag_NES_2-aa |
| | | MTKKFGTLTIHDTEKAAMAERPFQCRICMRN | | | linker_ZF |
| | | FSTSGSLSRHIRTHTGEKPFACDICGRKFAQSG | | | array_13-aa |
| | | SLTRHTKIHTGQKPFQCRICMRNFSRSDALSQ | | | Gly/Ser-rich |
| | | HTKIHLRGSGGGGSGGSGGSGSYALGPYQISA | | | flexible |
| | | PQLPAYNGQTVGTFYYVNDAGGLESKVFSSG | | | linker_DddA^{N}_4- |
| | | GPTPYPNYANAGHVEGQSALFMRDNGISEGL | | | aa linker_UGI |
| | | VFHNNPEGTCGFCVNMTETLLPENAKMTVVP | | | |
| | | PEGSGGSTNLSDIIEKETGKQLVIQESILMLPEE | | | |
| | | VEEVIGNKPESDILVHTAYDESTDENVMLLTS | | | |
| | | DAPEYKPWALVIQDSNGENKIKML (SEQ ID | | | |
| | | NO: 349) | | | |
| R8-3i- | v4 | MLGFVGRVAAAPASGALRRLTPSASLPPAQL | Canonical | DddA^{N} | MTS_FLAG |
| ATP8 | | LLRAAPTAVHPVRDYAAQDYKDDDDKVDE | | | tag_NES_2-aa |
| | | MTKKFGTLTIHDTEKGSLQKKLEELELDAAM | | | linker_NES_2-aa |
| | | AERPFQCRICMRNFSTSGSLSRHIRTHTGEKPF | | | linker_ZF |
| | | ACDICGRKFAQSGSLTRHTKIHTGQKPFQCRI | | | array_13-aa |
| | | CMRNFSRSDALSQHTKIHLRGSGGGGSGGSG | | | Gly/Ser-rich |
| | | GSGSYALGPYQISAPQLPAYNGQTVGTFYYV | | | flexible |
| | | NDAGGLESKVFSSGGPTPYPNYANAGHVEGQ | | | linker_DddA^{N}_4- |
| | | SALFMRDNGISEGLVFHNNPEGTCGFCVNMT | | | aa linker_UGI |
| | | ETLLPENAKMTVVPPEGSGGSTNLSDIIEKET | | | |
| | | GKQLVIQESILMLPEEVEEVIGNKPESDILVHT | | | |
| | | AYDESTDENVMLLTSDAPEYKPWALVIQDSN | | | |
| | | GENKIKML(SEQ ID NO: 359) | | | |
| R8-3i- | v5 | MLGFVGRVAAAPASGALRRLTPSASLPPAQL | Canonical | DddA^{N} | MTS_FLAG |
| ATP8 | | LLRAAPTAVHPVRDYAAQDYKDDDDKVDE | | | tag_NES_2-aa |
| | | MTKKFGTLTIHDTEKGSLQKKLEELELDAAM | | | linker_NES_2-aa |
| | | AERPFQCRICMRNFSTSGSLSRHIRTHTGEKPF | | | linker_ZF |
| | | ACDICGRKFAQSGSLTRHTKIHTGQKPFQCRI | | | array_13-aa |
| | | CMRNFSRSDALSQHTKIHLRGSGGGGSGGSG | | | Gly/Ser-rich |
| | | GSGSYALGPYQISAPQLPAYNGQTVGTFYYV | | | flexible |
| | | NDAGGLESKVFSSGGPTPYPNYANAGHVEGQ | | | linker_DddA^{N}_4- |
| | | SALFMRDNGISEGLVFHNNPEGTCGFCVNMT | | | aa |
| | | ETLLPENAKMTVVPPEGSGGSTNLSDIIEKET | | | linker_UGI_P2A_ |
| | | GKQLVIQESILMLPEEVEEVIGNKPESDILVHT | | | MTS_UGI |
| | | AYDESTDENVMLLTSDAPEYKPWALVIQDSN | | | |
| | | GENKIKMLGSGATNFSLLKQAGDVEENPGPM | | | |
| | | ASVLTPLLLRGLTGSARRLPVPRAKIHSLGST | | | |
| | | NLSDIIEKETGKQLVIQESILMLPEEVEEVIGN | | | |
| | | KPESDILVHTAYDESTDENVMLLTSDAPEYKP | | | |
| | | WALVIQDSNGENKIKML (SEQ ID NO: 511) | | | |
| R8-3i- | v6 | MLGFVGRVAAAPASGALRRLTPSASLPPAQL | V2 | DddA^{N} | MTS_FLAG |
| ATP8 | | LLRAAPTAVHPVRDYAAQDYKDDDDKVDE | | | tag_NES_2-aa |
| | | MTKKFGTLTIHDTEKGSLQKKLEELELDAAM | | | linker_NES_2-aa |
| | | AERPYKCEECGKAFNTSGSLSRHMKIHTGEK | | | linker_ZF array |
| | | PYKCEECGKAFNQSGSLTRHMKIHTGEKPYK | | | (optimized ZF |
| | | CEECGKAFNRSDALSQHMKIHLRGSGGGGSG | | | scaffold)_13-aa |
| | | GSGGSGSYALGPYQISAPQLPAYNGQTVGTF | | | Gly/Ser-rich |
| | | YYVNDAGGLESKVFSSGGPTPYPNYANAGH | | | flexible |
| | | VEGQSALFMRDNGISEGLVFHNNPEGTCGFC | | | linker_DddA^{N}_4- |
| | | VNMTETLLPENAKMTVVPPEGSGGSTNLSDII | | | aa |
| | | EKETGKQLVIQESILMLPEEVEEVIGNKPESDI | | | linker_UGI_P2A_ |
| | | LVHTAYDESTDENVMLLTSDAPEYKPWALVI | | | MTS_UGI |
| | | QDSNGENKIKMLGSGATNFSLLKQAGDVEEN | | | |
| | | PGPMASVLTPLLLRGLTGSARRLPVPRAKIHS | | | |
| | | LGSTNLSDIIEKETGKQLVIQESILMLPEEVEE | | | |
| | | VIGNKPESDILVHTAYDESTDENVMLLTSDAP | | | |
| | | EYKPWALVIQDSNGENKIKML (SEQ ID NO: | | | |
| | | 512) | | | |
| R8-3i- | v7 | MLGFVGRVAAAPASGALRRLTPSASLPPAQL | V2 | DddA^{N} | MTS_FLAG |
| ATP8 | | LLRAAPTAVHPVRDYAAQDYKDDDDKVDE | | | tag_NES_2-aa |
| | | MTKKFGTLTIHDTEKGSLQKKLEELELDAAM | | | linker_NES_2-aa |
| | | AERPYKCEECGKAFNTSGSLSRHMKIHTGEK | | | linker_ZF array |
| | | PYKCEECGKAFNQSGSLTRHMKIHTGEKPYK | | | (optimized ZF |
| | | CEECGKAFNRSDALSQHMKIHLRGSGGGGSG | | | scaffold)_13-aa |
| | | GSGGSGSYALGPYQISAPQLPAYNGQTVGTF | | | Gly/Ser-rich |
| | | YYVNDAGGLESKVFSSGGPTPYPNYANAGH | | | flexible |
| | | VEGQSALFMRDNGISEGLVFHNNPEGTCGFC | | | linker_DddA^{N} |
| | | VNMIETLLPENAKMTVVPPKGSGGSTNLSDII | | | (T1380I, E1396K) |
| | | EKETGKQLVIQESILMLPEEVEEVIGNKPESDI | | | 4-aa |
| | | LVHTAYDESTDENVMLLTSDAPEYKPWALVI | | | linker_UGI_P2A_ |
| | | QDSNGENKIKMLGSGATNFSLLKQAGDVEEN | | | MTS_UGI |
| | | PGPMASVLTPLLLRGLTGSARRLPVPRAKIHS | | | |
| | | LGSTNLSDIIEKETGKQLVIQESILMLPEEVEE | | | |
| | | VIGNKPESDILVHTAYDESTDENVMLLTSDAP | | | |
| | | EYKPWALVIQDSNGENKIKML (SEQ ID NO: | | | |
| | | 513) | | | |
| R8-3i- | v8 | MLGFVGRVAAAPASGALRRLTPSASLPPAQL | V2 | DddA^{N} | MTS_FLAG |
| ATP8 | | LLRAAPTAVHPVRDYAAQDYKDDDDKVDE | | | tag_NES_2-aa |
| | | MTKKFGTLTIHDTEKGSLQKKLEELELDAAM | | | linker_NES_2-aa |
| | | AERPYKCEECGKAFNTSGSLSRHMKIHTGEK | | | linker_ZF array |
| | | PYKCEECGKAFNQSGSLTRHMKIHTGEKPYK | | | (optimized ZF |
| | | CEECGKAFNRSDALSQHMKIHLRGSGGGGSG | | | scaffold)_13-aa |
| | | GSGGSGSYALGPYQISAPQLPAYNGQTVGTF | | | Gly/Ser-rich |
| | | YYVNDAGGLESKVFSSGGPTPYPNYANAGH | | | flexible |
| | | VEGQSALFMRDNGISEGLVFHNNPEGTCGFC | | | linker_DddA^{N} |
| | | VNMIETLLPENAKMTVVPPKGSGGSTNLSDII | | | (T1380I, E1396K)_ |
| | | EKETGKQLVIQESILMLPEEVEEVIGNKPESDI | | | 4-aa linker_UGI |
| | | LVHTAYDESTDENVMLLTSDAPEYKPWALVI | | | |
| | | QDSNGENKIKML (SEQ ID NO: 514) | | | |
| 4-3i- | v1 | MLGFVGRVAAAPASGALRRLTPSASLPPAQL | Canonical | DddA^{C} | MTS_NES_2-aa |
| ATP8 | | LLRAAPTAVHPVRDYAAQTSVDEMTKKFGT | | | linker_ZF |
| | | LTIHDTEKAAMAERPFQCRICMRNFSQASNLI | | | array_2-aa |
| | | SHIRTHTGEKPFACDICGRKFATSHSLTEHTKI | | | linker_DddA^{C}_4- |
| | | HTGQKPFQCRICMRNFSERSHLREHTKIHLRG | | | aa linker_UGI |
| | | SAIPVKRGATGETKVFTGNSNSPKSPTKGGCS | | | |
| | | GGSTNLSDIIEKETGKQLVIQESILMLPEEVEE | | | |
| | | VIGNKPESDILVHTAYDESTDENVMLLTSDAP | | | |
| | | EYKPWALVIQDSNGENKIKML (SEQ ID NO: | | | |
| | | 515) | | | |
| 4-3i- | v2 | MLGFVGRVAAAPASGALRRLTPSASLPPAQL | Canonical | DddA^{C} | MTS_NES_2-aa |
| ATP8 | | LLRAAPTAVHPVRDYAAQTSVDEMTKKFGT | | | linker_ZF |
| | | LTIHDTEKAAMAERPFQCRICMRNFSQASNLI | | | array_13-aa |
| | | SHIRTHTGEKPFACDICGRKFATSHSLTEHTKI | | | Gly/Ser-rich |
| | | HTGQKPFQCRICMRNFSERSHLREHTKIHLRG | | | flexible |
| | | SGGGGSGGSGGSAIPVKRGATGETKVFTGNS | | | linker_DddA^{C}_4- |
| | | NSPKSPTKGGCSGGSTNLSDIIEKETGKQLVIQ | | | aa linker_UGI |
| | | ESILMLPEEVEEVIGNKPESDILVHTAYDESTD | | | |
| | | ENVMLLTSDAPEYKPWALVIQDSNGENKIKM | | | |
| | | L (SEQ ID NO: 516) | | | |
| 4-3i- | v3 | MLGFVGRVAAAPASGALRRLTPSASLPPAQL | Canonical | DddA^{C} | MTS_FLAG |
| ATP8 | | LLRAAPTAVHPVRDYAAQDYKDDDDKVDE | | | tag_NES_2-aa |
| | | MTKKFGTLTIHDTEKAAMAERPFQCRICMRN | | | linker_ZF |
| | | FSQASNLISHIRTHTGEKPFACDICGRKFATSH | | | array_13-aa |
| | | SLTEHTKIHTGQKPFQCRICMRNFSERSHLRE | | | Gly/Ser-rich |
| | | HTKIHLRGSGGGGSGGSGGSAIPVKRGATGET | | | flexible |
| | | KVFTGNSNSPKSPTKGGCSGGSTNLSDIIEKET | | | linker_DddA^{C}_4- |
| | | GKQLVIQESILMLPEEVEEVIGNKPESDILVHT | | | aa linker_UGI |
| | | AYDESTDENVMLLTSDAPEYKPWALVIQDSN | | | |
| | | GENKIKML (SEQ ID NO: 517) | | | |
| 4-3i- | v4 | MLGFVGRVAAAPASGALRRLTPSASLPPAQL | Canonical | DddA^{C} | MTS_FLAG |
| ATP8 | | LLRAAPTAVHPVRDYAAQDYKDDDDKVDE | | | tag_NES_2-aa |
| | | MTKKFGTLTIHDTEKGSLQKKLEELELDAAM | | | linker_NES_2-aa |
| | | AERPFQCRICMRNFSQASNLISHIRTHTGEKPF | | | linker ZF |
| | | ACDICGRKFATSHSLTEHTKIHTGQKPFQCRIC | | | array_13-aa |
| | | MRNFSERSHLREHTKIHLRGSGGGGSGGSGG | | | Gly/Ser-rich |
| | | SAIPVKRGATGETKVFTGNSNSPKSPTKGGCS | | | flexible |
| | | GGSTNLSDIIEKETGKQLVIQESILMLPEEVEE | | | linker_DddA^{C}_4- |
| | | VIGNKPESDILVHTAYDESTDENVMLLTSDAP | | | aa linker_UGI |
| | | EYKPWALVIQDSNGENKIKML (SEQ ID NO: | | | |
| | | 518) | | | |
| 4-3i- | v5 | MLGFVGRVAAAPASGALRRLTPSASLPPAQL | Canonical | DddA^{C} | MTS_FLAG |
| ATP8 | | LLRAAPTAVHPVRDYAAQDYKDDDDKVDE | | | tag_NES_2-aa |
| | | MTKKFGTLTIHDTEKGSLQKKLEELELDAAM | | | linker_NES_2-aa |
| | | AERPFQCRICMRNFSQASNLISHIRTHTGEKPF | | | linker ZF |
| | | ACDICGRKFATSHSLTEHTKIHTGQKPFQCRIC | | | array_13-aa |
| | | MRNFSERSHLREHTKIHLRGSGGGGSGGSGG | | | Gly/Ser-rich |
| | | SAIPVKRGATGETKVFTGNSNSPKSPTKGGCS | | | flexible |
| | | GGSTNLSDIIEKETGKQLVIQESILMLPEEVEE | | | linker_DddA^{C}_4- |
| | | VIGNKPESDILVHTAYDESTDENVMLLTSDAP | | | aa |
| | | EYKPWALVIQDSNGENKIKMLGSGATNFSLL | | | linker_UGI_P2A_ |
| | | KQAGDVEENPGPMASVLTPLLLRGLTGSARR | | | MTS_UGI |
| | | LPVPRAKIHSLGSTNLSDIIEKETGKQLVIQESI | | | |
| | | LMLPEEVEEVIGNKPESDILVHTAYDESTDEN | | | |
| | | VMLLTSDAPEYKPWALVIQDSNGENKIKML | | | |
| | | (SEQ ID NO: 519) | | | |
| 4-3i- | v6 | MLGFVGRVAAAPASGALRRLTPSASLPPAQL | V2 | DddA^{C} | MTS_FLAG |
| ATP8 | | LLRAAPTAVHPVRDYAAQDYKDDDDKVDE | | | tag_NES_2-aa |
| | | MTKKFGTLTIHDTEKGSLQKKLEELELDAAM | | | linker_NES_2-aa |
| | | AERPYKCEECGKAFNQASNLISHMKIHTGEKP | | | linker_ZF array |
| | | YKCEECGKAFNTSHSLTEHMKIHTGEKPYKC | | | (optimized ZF |
| | | EECGKAFNERSHLREHMKIHLRGSGGGGSGG | | | scaffold)_13-aa |
| | | SGGSAIPVKRGATGETKVFTGNSNSPKSPTKG | | | Gly/Ser-rich |
| | | GCSGGSTNLSDIIEKETGKQLVIQESILMLPEE | | | flexible |
| | | VEEVIGNKPESDILVHTAYDESTDENVMLLTS | | | linker_DddA^{C}_4- |
| | | DAPEYKPWALVIQDSNGENKIKMLGSGATNF | | | aa |
| | | SLLKQAGDVEENPGPMASVLTPLLLRGLTGS | | | linker_UGI_P2A_ |
| | | ARRLPVPRAKIHSLGSTNLSDIIEKETGKQLVI | | | MTS_UGI |
| | | QESILMLPEEVEEVIGNKPESDILVHTAYDEST | | | |
| | | DENVMLLTSDAPEYKPWALVIQDSNGENKIK | | | |
| | | ML (SEQ ID NO: 520) | | | |
| 4-3i- | v7 | MLGFVGRVAAAPASGALRRLTPSASLPPAQL | V2 | DddA^{C} | MTS_FLAG |
| ATP8 | | LLRAAPTAVHPVRDYAAQDYKDDDDKVDE | | | tag_NES_2-aa |
| | | MTKKFGTLTIHDTEKGSLQKKLEELELDAAM | | | linker_NES_2-aa |
| | | AERPYKCEECGKAFNQASNLISHMKIHTGEKP | | | linker_ZF array |
| | | YKCEECGKAFNTSHSLTEHMKIHTGEKPYKC | | | (optimized ZF |
| | | EECGKAFNERSHLREHMKIHLRGSGGGGSGG | | | scaffold)_13-aa |
| | | SGGSAIPVKRGATGETKVFIGNSNSPKSPTKG | | | Gly/Ser-rich |
| | | GCSGGSTNLSDIIEKETGKQLVIQESILMLPEE | | | flexible |
| | | VEEVIGNKPESDILVHTAYDESTDENVMLLTS | | | linker_DddAC |
| | | DAPEYKPWALVIQDSNGENKIKMLGSGATNF | | | (T1413I)_4-aa |
| | | SLLKQAGDVEENPGPMASVLTPLLLRGLTGS | | | linker_UGI_P2A |
| | | ARRLPVPRAKIHSLGSTNLSDIIEKETGKQLVI | | | MTS_UGI |
| | | QESILMLPEEVEEVIGNKPESDILVHTAYDEST | | | |
| | | DENVMLLTSDAPEYKPWALVIQDSNGENKIK | | | |
| | | ML (SEQ ID NO: 521) | | | |
| 4-3i- | v8 | MLGFVGRVAAAPASGALRRLTPSASLPPAQL | V2 | DddA^{C} | MTS_FLAG |
| ATP8 | | LLRAAPTAVHPVRDYAAQDYKDDDDKVDE | | | tag_NES_2-aa |
| | | MTKKFGTLTIHDTEKGSLQKKLEELELDAAM | | | linker_NES_2-aa |
| | | AERPYKCEECGKAFNQASNLISHMKIHTGEKP | | | linker_ZF array |
| | | YKCEECGKAFNTSHSLTEHMKIHTGEKPYKC | | | (optimized ZF |
| | | EECGKAFNERSHLREHMKIHLRGSGGGGSGG | | | scaffold)_13-aa |
| | | SGGSAIPVKRGATGETKVFIGNSNSPKSPTKG | | | Gly/Ser-rich |
| | | GCSGGSTNLSDIIEKETGKQLVIQESILMLPEE | | | flexible |
| | | VEEVIGNKPESDILVHTAYDESTDENVMLLTS | | | linker_DddA^{C} |
| | | DAPEYKPWALVIQDSNGENKIKML (SEQ ID | | | (T1413I)_4-aa |
| | | NO: 522) | | | linker_UGI |

TABLE 2
Mitochondrial ZF-DdCBEs, N-terminal architecture

| | Archi- | | ZF | DddA | Composition (N- |
|---|---|---|---|---|---|
| Name | tecture | Sequence | scaffold | split | to C-terminus) |
| G35- | v1 | MLGFVGRVAAAPASGALRRLTPSASLPPAQLL | Canonical | DddA^{N} | MTS_NES_6-aa |
| V1 | | LRAAPTAVHPVRDYAAQVDEMTKKFGTLTIH | | | linker_DddA^{N}_2- |
| | | DTEKAASGGSGSYALGPYQISAPQLPAYNGQT | | | aa linker ZF |
| | | VGTFYYVNDAGGLESKVFSSGGPTPYPNYANA | | | array_10-aa |
| | | GHVEGQSALFMRDNGISEGLVFHNNPEGTCGF | | | linker_UGI |
| | | CVNMTETLLPENAKMTVVPPEGGSMAERPFQ | | | |
| | | CRICMRNFSRSDNLVRHIRTHTGEKPFACDICG | | | |
| | | RKFAQSSSLVRHTKIHTGQKPFQCRICMRNFST | | | |
| | | SGNLVRHTKIHLRSGGSGGSGGSTNLSDIIEKET | | | |
| | | GKQLVIQESILMLPEEVEEVIGNKPESDILVHTA | | | |
| | | YDESTDENVMLLTSDAPEYKPWALVIQDSNGE | | | |
| | | NKIKML (SEQ ID NO: 523) | | | |
| G35- | v5 | MLGFVGRVAAAPASGALRRLTPSASLPPAQLL | Canonical | DddA^{N} | MTS_FLAG |
| V1 | | LRAAPTAVHPVRDYAAQDYKDDDDKVDEMT | | | tag_NES_2-aa |
| | | KKFGTLTIHDTEKGSLQKKLEELELDAASGGS | | | linker_NES_6-aa |
| | | GSYALGPYQISAPQLPAYNGQTVGTFYYVNDA | | | linker DddA^{N}_13- |
| | | GGLESKVFSSGGPTPYPNYANAGHVEGQSALF | | | aa Gly/Ser-rich |
| | | MRDNGISEGLVFHNNPEGTCGFCVNMTETLLP | | | flexible linker_ |
| | | ENAKMTVVPPEGGSGGGGSGGSGGSMAERPF | | | ZF array_10-aa |
| | | QCRICMRNFSRSDNLVRHIRTHTGEKPFACDIC | | | linker_UGI_P2A |
| | | GRKFAQSSSLVRHTKIHTGQKPFQCRICMRNFS | | | MTS_UGI |
| | | TSGNLVRHTKIHLRSGGSGGSGGSTNLSDIIEKE | | | |
| | | TGKQLVIQESILMLPEEVEEVIGNKPESDILVHT | | | |
| | | AYDESTDENVMLLTSDAPEYKPWALVIQDSNG | | | |
| | | ENKIKMLGSGATNFSLLKQAGDVEENPGPMAS | | | |
| | | VLTPLLLRGLTGSARRLPVPRAKIHSLGSTNLS | | | |
| | | DIIEKETGKQLVIQESILMLPEEVEEVIGNKPES | | | |
| | | DILVHTAYDESTDENVMLLTSDAPEYKPWALV | | | |
| | | IQDSNGENKIKML (SEQ ID NO: 524) | | | |
| G35- | v6 | MLGFVGRVAAAPASGALRRLTPSASLPPAQLL | V20 | DddA^{N} | MTS_FLAG |
| V1 | | LRAAPTAVHPVRDYAAQDYKDDDDKVDEMT | | | tag_NES_2-aa |
| | | KKFGTLTIHDTEKGSLQKKLEELELDAASGGS | | | linker_NES_6-aa |
| | | GSYALGPYQISAPQLPAYNGQTVGTFYYVNDA | | | linker_DddA^{N}_13- |
| | | GGLESKVFSSGGPTPYPNYANAGHVEGQSALF | | | aa Gly/Ser-rich |
| | | MRDNGISEGLVFHNNPEGTCGFCVNMTETLLP | | | flexible linker_ZF |
| | | ENAKMTVVPPEGGSGGGGSGGSGGSMAERPY | | | array (optimized |
| | | KCEECGKAFNRSDNLVRHMKIHTGEKPYKCEE | | | ZF scaffold)_10- |
| | | CGKAFNQSSSLVRHMKIHTGEKPYKCEECGKA | | | aa |
| | | FNTSGNLVRHTKIHLRSGGSGGSGGSTNLSDIIE | | | linker_UGI_P2A_ |
| | | KETGKQLVIQESILMLPEEVEEVIGNKPESDILV | | | MTS_UGI |
| | | HTAYDESTDENVMLLTSDAPEYKPWALVIQDS | | | |
| | | NGENKIKMLGSGATNFSLLKQAGDVEENPGP | | | |
| | | MASVLTPLLLRGLTGSARRLPVPRAKIHSLGST | | | |
| | | NLSDIIEKETGKQLVIQESILMLPEEVEEVIGNK | | | |
| | | PESDILVHTAYDESTDENVMLLTSDAPEYKPW | | | |
| | | ALVIQDSNGENKIKML (SEQ ID NO: 525) | | | |
| G35- | v7 | MLGFVGRVAAAPASGALRRLTPSASLPPAQLL | V20 | DddA^{N} | MTS_FLAG |
| V1 | | LRAAPTAVHPVRDYAAQDYKDDDDKVDEMT | | | tag_NES_2-aa |
| | | KKFGTLTIHDTEKGSLQKKLEELELDAASGGS | | | linker_NES_6-aa |
| | | GSYALGPYQISAPQLPAYNGQTVGTFYYVNDA | | | linker_DddA^{N} |
| | | GGLESKVFSSGGPTPYPNYANAGHVEGQSALF | | | (T1380I, E1396K) |
| | | MRDNGISEGLVFHNNPEGTCGFCVNMIETLLPE | | | 13-aa Gly/Ser- |
| | | NAKMTVVPPKGGSGGGGSGGSGGSMAERPYK | | | rich flexible |
| | | CEECGKAFNRSDNLVRHMKIHTGEKPYKCEEC | | | linker_ZF array |
| | | GKAFNQSSSLVRHMKIHTGEKPYKCEECGKAF | | | (optimized ZF |
| | | NTSGNLVRHTKIHLRSGGSGGSGGSTNLSDIIE | | | scaffold)_10-aa |
| | | KETGKQLVIQESILMLPEEVEEVIGNKPESDILV | | | linker_UGI_P2A_ |
| | | HTAYDESTDENVMLLTSDAPEYKPWALVIQDS | | | MTS_UGI |
| | | NGENKIKMLGSGATNFSLLKQAGDVEENPGP | | | |
| | | MASVLTPLLLRGLTGSARRLPVPRAKIHSLGST | | | |
| | | NLSDIIEKETGKQLVIQESILMLPEEVEEVIGNK | | | |
| | | PESDILVHTAYDESTDENVMLLTSDAPEYKPW | | | |
| | | ALVIQDSNGENKIKML (SEQ ID NO: 526) | | | |
| G35- | v8 | MLGFVGRVAAAPASGALRRLTPSASLPPAQLL | V20 | DddA^{N} | MTS_FLAG |
| V1 | | LRAAPTAVHPVRDYAAQDYKDDDDKVDEMT | | | tag_NES_2-aa |
| | | KKFGTLTIHDTEKGSLQKKLEELELDAASGGS | | | linker_NES_6-aa |
| | | GSYALGPYQISAPQLPAYNGQTVGTFYYVNDA | | | linker_DddAN |
| | | GGLESKVFSSGGPTPYPNYANAGHVEGQSALF | | | (T1380I, E1396K) |
| | | MRDNGISEGLVFHNNPEGTCGFCVNMIETLLPE | | | 13-aa Gly/Ser- |
| | | NAKMTVVPPKGGSGGGGSGGSGGSMAERPYK | | | rich flexible |
| | | CEECGKAFNRSDNLVRHMKIHTGEKPYKCEEC | | | linker_ZF array |
| | | GKAFNQSSSLVRHMKIHTGEKPYKCEECGKAF | | | (optimized ZF |
| | | NTSGNLVRHTKIHLRSGGSGGSGGSTNLSDIIE | | | scaffold)_10-aa |
| | | KETGKQLVIQESILMLPEEVEEVIGNKPESDILV | | | linker_UGI |
| | | HTAYDESTDENVMLLTSDAPEYKPWALVIQDS | | | |
| | | NGENKIKML (SEQ ID NO: 527) | | | |
| G36- | v1 | MLGFVGRVAAAPASGALRRLTPSASLPPAQLL | Canonical | DddA^{C} | MTS_NES_6-aa |
| V5 | | LRAAPTAVHPVRDYAAQVDEMTKKFGTLTIH | | | linker_DddA^{C}_2- |
| | | DTEKAASGGSAIPVKRGATGETKVFTGNSNSP | | | aa linker_ZF |
| | | KSPTKGGCGSMAERPFQCRICMRNFSQSSNLV | | | array_10-aa |
| | | RHIRTHTGEKPFACDICGRKFATSGHLVRHTKI | | | linker_UGI |
| | | HTGQKPFQCRICMRNFSRSDELVRHTKIHLRSG | | | |
| | | GSGGSGGSTNLSDIIEKETGKQLVIQESILMLPE | | | |
| | | EVEEVIGNKPESDILVHTAYDESTDENVMLLTS | | | |
| | | DAPEYKPWALVIQDSNGENKIKML (SEQ ID | | | |
| | | NO: 528) | | | |
| G36- | v5 | MLGFVGRVAAAPASGALRRLTPSASLPPAQLL | Canonical | DddA^{C} | MTS_FLAG |
| V5 | | LRAAPTAVHPVRDYAAQDYKDDDDKVDEMT | | | tag_NES_2-aa |
| | | KKFGTLTIHDTEKGSLQKKLEELELDAASGGS | | | linker_NES_6-aa |
| | | AIPVKRGATGETKVFTGNSNSPKSPTKGGCGS | | | linker_DddA^{C}_13- |
| | | GGGGSGGSGGSMAERPFQCRICMRNFSQSSNL | | | aa Gly/Ser-rich |
| | | VRHIRTHTGEKPFACDICGRKFATSGHLVRHTK | | | flexible linker_ZF |
| | | IHTGQKPFQCRICMRNFSRSDELVRHTKIHLRS | | | array_10-aa |
| | | GGSGGSGGSTNLSDIIEKETGKQLVIQESILMLP | | | linker_UGI_P2A_ |
| | | EEVEEVIGNKPESDILVHTAYDESTDENVMLLT | | | MTS_UGI |
| | | SDAPEYKPWALVIQDSNGENKIKMLGSGATNF | | | |
| | | SLLKQAGDVEENPGPMASVLTPLLLRGLTGSA | | | |
| | | RRLPVPRAKIHSLGSTNLSDIIEKETGKQLVIQE | | | |
| | | SILMLPEEVEEVIGNKPESDILVHTAYDESTDEN | | | |
| | | VMLLTSDAPEYKPWALVIQDSNGENKIKML | | | |
| | | (SEQ ID NO: 529) | | | |
| G36- | v6 | MLGFVGRVAAAPASGALRRLTPSASLPPAQLL | AGKS | DddA^{C} | MTS_FLAG |
| V5 | | LRAAPTAVHPVRDYAAQDYKDDDDKVDEMT | | | tag_NES_2-aa |
| | | KKFGTLTIHDTEKGSLQKKLEELELDAASGGS | | | linker_NES_6-aa |
| | | AIPVKRGATGETKVFTGNSNSPKSPTKGGCGS | | | linker_DddA^{C}_13- |
| | | GGGGSGGSGGSMAERPYACPECGKSFSQSSNL | | | aa Gly/Ser-rich |
| | | VRHIRTHTGEKPYACPECGKSFSTSGHLVRHIR | | | flexible linker_ZF |
| | | THTGEKPYACPECGKSFSRSDELVRHTKIHLRS | | | array (optimized |
| | | GGSGGSGGSTNLSDIIEKETGKQLVIQESILMLP | | | ZF scaffold)_10- |
| | | EEVEEVIGNKPESDILVHTAYDESTDENVMLLT | | | aa |
| | | SDAPEYKPWALVIQDSNGENKIKMLGSGATNF | | | linker_UGI_P2A |
| | | SLLKQAGDVEENPGPMASVLTPLLLRGLTGSA | | | MTS_UGI |
| | | RRLPVPRAKIHSLGSTNLSDIIEKETGKQLVIQE | | | |
| | | SILMLPEEVEEVIGNKPESDILVHTAYDESTDEN | | | |
| | | VMLLTSDAPEYKPWALVIQDSNGENKIKML | | | |
| | | (SEQ ID NO: 530) | | | |
| G36- | v7 | MLGFVGRVAAAPASGALRRLTPSASLPPAQLL | AGKS | DddA^{C} | MTS_FLAG |
| V5 | | LRAAPTAVHPVRDYAAQDYKDDDDKVDEMT | | | tag_NES_2-aa |
| | | KKFGTLTIHDTEKGSLQKKLEELELDAASGGS | | | linker_NES_6-aa |
| | | AIPVKRGATGETKVFIGNSNSPKSPTKGGCGSG | | | linker_DddA^{C} |
| | | GGGSGGSGGSMAERPYACPECGKSFSQSSNLV | | | (T1413I)_13-aa |
| | | RHIRTHTGEKPYACPECGKSFSTSGHLVRHIRT | | | Gly/Ser-rich |
| | | HTGEKPYACPECGKSFSRSDELVRHTKIHLRSG | | | flexible linker_ZF |
| | | GSGGSGGSTNLSDIIEKETGKQLVIQESILMLPE | | | array (optimized |
| | | EVEEVIGNKPESDILVHTAYDESTDENVMLLTS | | | ZF scaffold)_10- |
| | | DAPEYKPWALVIQDSNGENKIKMLGSGATNFS | | | aa |
| | | LLKQAGDVEENPGPMASVLTPLLLRGLTGSAR | | | linker_UGI_P2A |
| | | RLPVPRAKIHSLGSTNLSDIIEKETGKQLVIQESI | | | MTS_UGI |
| | | LMLPEEVEEVIGNKPESDILVHTAYDESTDENV | | | |
| | | MLLTSDAPEYKPWALVIQDSNGENKIKML | | | |
| | | (SEQ ID NO: 531) | | | |
| G36- | v8 | MLGFVGRVAAAPASGALRRLTPSASLPPAQLL | AGKS | DddA^{C} | MTS_FLAG |
| V5 | | LRAAPTAVHPVRDYAAQDYKDDDDKVDEMT | | | tag_NES_2-aa |
| | | KKFGTLTIHDTEKGSLQKKLEELELDAASGGS | | | linker_NES_6-aa |
| | | AIPVKRGATGETKVFIGNSNSPKSPTKGGCGSG | | | linker_DddA^{C} |
| | | GGGSGGSGGSMAERPYACPECGKSFSQSSNLV | | | (T1413I)_13-aa |
| | | RHIRTHTGEKPYACPECGKSFSTSGHLVRHIRT | | | Gly/Ser-rich |
| | | HTGEKPYACPECGKSFSRSDELVRHTKIHLRSG | | | flexible linker_ZF |
| | | GSGGSGGSTNLSDIIEKETGKQLVIQESILMLPE | | | array (optimized |
| | | EVEEVIGNKPESDILVHTAYDESTDENVMLLTS | | | ZF scaffold)_10- |
| | | DAPEYKPWALVIQDSNGENKIKML (SEQ ID | | | aa linker_UGI |
| | | NO: 532) | | | |

TABLE 3
Nuclear ZF-DdCBEs, canonical architecture

| | | | | Composition |
|---|---|---|---|---|
| | | ZF | DddA | (N- to C- |
| Name | Sequence | scaffold | split | terminus) |
| 3xG22- | MKRTADGSEFESPKKKRKVSGSPAAK | X1 | DddA^{N} | 4xNLS_2-aa |
| COL5A1 | RVKLDSGSKRTADGSEFESPKKKRKVS | | | linker ZF |
| | GSPAAKRVKLDSGSAAMAERPFACDIC | | | array |
| | GRKFARSDNLVRHIRTHTGEKPFACDI | | | (optimized ZF |
| | CGRKFAREDNLHTHIRTHTGEKPFACD | | | scaffold)_13- |
| | ICGRKFARSDNLVRHTKIHLRGSGGGG | | | aa Gly/Ser- |
| | SGGSGGSGSYALGPYQISAPQLPAYNG | | | rich flexible |
| | QTVGTFYYVNDAGGLESKVFSSGGPTP | | | linker_DddA^{N} |
| | YPNYANAGHVEGQSALFMRDNGISEG | | | (T1380I,E139 |
| | LVFHNNPEGTCGFCVNMIETLLPENAK | | | 6K)_4-aa |
| | MTVVPPKGSGGSTNLSDIIEKETGKQL | | | linker_UGI |
| | VIQESILMLPEEVEEVIGNKPESDILVHT | | | |
| | AYDESTDENVMLLTSDAPEYKPWALV | | | |
| | IQDSNGENKIKML (SEQ ID NO: 533) | | | |
| 6xG34- | MKRTADGSEFESPKKKRKVSGSPAAK | X1 | DddA^{C} | 4xNLS_2-aa |
| COL5A1 | RVKLDSGSKRTADGSEFESPKKKRKVS | | | linker_ZF |
| | GSPAAKRVKLDSGSAAMAERPFACDIC | | | array |
| | GRKFARNDALTEHIRTHTGEKPFACDI | | | (optimized ZF |
| | CGRKFATSGELVRHIRTHTGEKPFACDI | | | scaffold)_13- |
| | CGRKFARTDTLRDHIRTHTGEKPFACD | | | aa Gly/Ser- |
| | ICGRKFADCRDLARHIRTHTGEKPFAC | | | rich flexible |
| | DICGRKFARSDNLVRHIRTHTGEKPFA | | | linker_DddA^{C} |
| | CDICGRKFARSDELVRHTKIHLRGSGG | | | (T1413I)_4-aa |
| | GGSGGSGGSAIPVKRGATGETKVFIGN | | | linker_UGI |
| | SNSPKSPTKGGCSGGSTNLSDIIEKETG | | | |
| | KQLVIQESILMLPEEVEEVIGNKPESDIL | | | |
| | VHTAYDESTDENVMLLTSDAPEYKPW | | | |
| | ALVIQDSNGENKIKML (SEQ ID NO: | | | |
| | 534) | | | |

TABLE 4
Nuclear ZF-DdCBEs, N-terminal architecture

| | | | | Composition |
|---|---|---|---|---|
| | | ZF | DddA | (N- to C- |
| Name | Sequence | scaffold | split | terminus) |
| LB32- | MKRTADGSEFESPKKKRKVSGSPAAK | X1 | DddA^{N} | 4xNLS_6-aa |
| HBB | RVKLDSGSKRTADGSEFESPKKKRKV | | | linker_DddA^{N} |
| | SGSPAAKRVKLDSGSAASGGSGSYAL | | | (T1380I, E1396K)_ |
| | GPYQISAPQLPAYNGQTVGTFYYVND | | | 13-aa Gly/Ser- |
| | AGGLESKVFSSGGPTPYPNYANAGHV | | | rich flexible |
| | EGQSALFMRDNGISEGLVFHNNPEGT | | | linker_ZF array |
| | CGFCVNMIETLLPENAKMTVVPPKGG | | | (optimized ZF |
| | SGGGGSGGSGGSMAERPFACDICGRK | | | scaffold)_10-aa |
| | FAQSGDLRRHIRTHTGEKPFACDICGR | | | linker_UGI |
| | KFARSDHLTTHIRTHTGEKPFACDICG | | | |
| | RKFADPGHLVRHTKIHLRSGGSGGSG | | | |
| | GSTNLSDIIEKETGKQLVIQESILMLPE | | | |
| | EVEEVIGNKPESDILVHTAYDESTDEN | | | |
| | VMLLTSDAPEYKPWALVIQDSNGENK | | | |
| | IKML (SEQ ID NO: 535) | | | |
| RB610- | MKRTADGSEFESPKKKRKVSGSPAAK | X1 | DddA^{C} | 4xNLS_6-aa |
| HBB | RVKLDSGSKRTADGSEFESPKKKRKV | | | linker_DddA^{C} |
| | SGSPAAKRVKLDSGSAASGGSAIPVK | | | (T1413I)_13-aa |
| | RGATGETKVFIGNSNSPKSPTKGGCGS | | | Gly/Ser-rich |
| | GGGGSGGSGGSMAERPFACDICGRKF | | | flexible |
| | ARLRDIQFHIRTHTGEKPFACDICGRK | | | linker_ZF array |
| | FADPGHLVRHIRTHTGEKPFACDICGR | | | (optimized ZF |
| | KFATSGNLVRHIRTHTGEKPFACDICG | | | scaffold)_10-aa |
| | RKFAQKSSLIAHIRTHTGEKPFACDIC | | | linker_UGI |
| | GRKFAQSGDLRRHIRTHTGEKPFACDI | | | |
| | CGRKFAQASNLISHTKIHLRSGGSGGS | | | |
| | GGSTNLSDIIEKETGKQLVIQESILMLP | | | |
| | EEVEEVIGNKPESDILVHTAYDESTDE | | | |
| | NVMLLTSDAPEYKPWALVIQDSNGEN | | | |
| | KIKML (SEQ ID NO: 536) | | | |

TABLE 5
Amplicons

| Amplicon | | | | Forward | Reverse |
|---|---|---|---|---|---|
| name | Sequence | Length | Species | primer | primer |
| ATP8 | CTTTACAGTGAAATGCCCCAA | 209 | Human | HTS_ | HTS_ |
| | CTAAATACTACCGTATGGCCC | | | ATP8_F | ATP8_R |
| | ACCATAATTACCCCCATACTC | | | | |
| | CTTACACTATTCCTCATCACC | | | | |
| | CAACTAAAAATATTAAACACA | | | | |
| | AACTACCACCTACCTCCCTCA | | | | |
| | CCAAAGCCCATAAAAATAAA | | | | |
| | AAATTATAACAAACCCTGAGA | | | | |
| | ACCAAAATGAACGAAAATCT | | | | |
| | GTTCGCTTCATTCATTGCCCCC | | | | |
| | (SEQ ID NO: 537) | | | | |
| ND51 | CGGGTCCATCATCCACAACCT | 210 | Human | HTS_ | HTS_ |
| | TAACAATGAACAAGATATTCG | | | ND51_F | ND51_R |
| | AAAAATAGGAGGACTACTCA | | | | |
| | AAACCATACCTCTCACTTCAA | | | | |
| | CCTCCCTCACCATTGGCAGCC | | | | |
| | TAGCATTAGCAGGAATACCTT | | | | |
| | TCCTCACAGGTTTCTACTCCA | | | | |
| | AAGACCACATCATCGAAACC | | | | |
| | GCAAACATATCATACACAAAC | | | | |
| | GCCTGAGCCCTATCTATTACT | | | | |
| | CT (SEQ ID NO: 538) | | | | |
| ND62 | AAAGTTTACCACAACCACCAC | 217 | Human | HTS_ | HTS_ |
| | CCCATCATACTCTTTCACCCA | | | ND62_F | ND62_R |
| | CAGCACCAATCCTACCTCCAT | | | | |
| | CGCTAACCCCACTAAAACACT | | | | |
| | CACCAAGACCTCAACCCCTGA | | | | |
| | CCCCCATGCCTCAGGATACTC | | | | |
| | CTCAATAGCCATCGCTGTAGT | | | | |
| | ATATCCAAAGACAACCATCAT | | | | |
| | TCCCCCTAAATAAATTAAAAA | | | | |
| | AACTATTAAACCCATATAACC | | | | |
| | TCCCCCA (SEQ ID NO: 539) | | | | |
| COX1 | CCTACTCCTGCTCGCATCTGC | 213 | Human | HTS_ | HTS_ |
| | TATAGTGGAGGCCGGAGCAG | | | COX1_F | COX1_R |
| | GAACAGGTTGAACAGTCTACC | | | | |
| | CTCCCTTAGCAGGGAACTACT | | | | |
| | CCCACCCTGGAGCCTCCGTAG | | | | |
| | ACCTAACCATCTTCTCCTTAC | | | | |
| | ACCTAGCAGGTGTCTCCTCTA | | | | |
| | TCTTAGGGGCCATCAATTTCA | | | | |
| | TCACAACAATTATCAATATAA | | | | |
| | AACCCCCTGCCATAACCCAAT | | | | |
| | ACCA (SEQ ID NO: 540) | | | | |
| *V1 | GGCTATATACAACTACGCAAA | 226 | Human | HTS_ | HTS_ |
| | GGCCCCAACGTTGTAGGCCCC | | | V1F | V1R |
| | TACGGGCTACTACAACCCTTC | | | | |
| | GCTGACGCCATAAAACTCTTC | | | | |
| | ACCAAAGAGCCCCTAAAACC | | | | |
| | CGCCACATCTACCATCACCCT | | | | |
| | CTACATCACCGCCCCGACCTT | | | | |
| | AGCTCTCACCATCGCTCTTCT | | | | |
| | ACTATGAACCCCCCTCCCCAT | | | | |
| | ACCCAACCCCCTGGTCAACCT | | | | |
| | CAACCTAGGCCTCCTAT (SEQ | | | | |
| | ID NO: 541) | | | | |
| **V2 | AATCGGAGGCTTTGGCAACTG | 217 | Human | HTS_ | HTS_ |
| | ACTAGTTCCCCTAATAATCGG | | | V2F | V2R |
| | TGCCCCCGATATGGCGTTTCC | | | | |
| | CCGCATAAACAACATAAGCTT | | | | |
| | CTGACTCTTACCTCCCTCTCTC | | | | |
| | CTACTCCTGCTCGCATCTGCT | | | | |
| | ATAGTGGAGGCCGGAGCAGG | | | | |
| | AACAGGTTGAACAGTCTACCC | | | | |
| | TCCCTTAGCAGGGAACTACTC | | | | |
| | CCACCCTGGAGCCTCCGTAGA | | | | |
| | CCTAACC (SEQ ID NO: 542) | | | | |
| #V3 | ATACCAAACGCCCCTCTTCGT | 222 | Human | HTS_ | HTS_ |
| | CTGATCCGTCCTAATCACAGC | | | V3F | V3R |
| | AGTCCTACTTCTCCTATCTCTC | | | | |
| | CCAGTCCTAGCTGCTGGCATC | | | | |
| | ACTATACTACTAACAGACCGC | | | | |
| | AACCTCAACACCACCTTCTTC | | | | |
| | GACCCCGCCGGAGGAGGAGA | | | | |
| | CCCCATTCTATACCAACACCT | | | | |
| | ATTCTGATTTTTCGGTCACCCT | | | | |
| | GAAGTTTATATTCTTATCCTA | | | | |
| | CCAGGCTTCGG (SEQ ID NO: | | | | |
| | 543) | | | | |
| #V4 | GCATCCTTTACATAACAGACG | 213 | Human | HTS_ | HTS_ |
| | AGGTCAACGATCCCTCCCTTA | | | V4F | V4R |
| | CCATCAAATCAATTGGCCACC | | | | |
| | AATGGTACTGAACCTACGAGT | | | | |
| | ACACCGACTACGGCGGACTA | | | | |
| | ATCTTCAACTCCTACATACTT | | | | |
| | CCCCCATTATTCCTAGAACCA | | | | |
| | GGCGACCTGCGACTCCTTGAC | | | | |
| | GTTGACAATCGAGTAGTACTC | | | | |
| | CCGATTGAAGCCCCCATTCGT | | | | |
| | ATAA (SEQ ID NO: 544) | | | | |
| ^{∧}V5 | CCCATAATCATACAAAGCCCC | 228 | Human | HTS_ | HTS_ |
| | CGCACCAATAGGATCCTCCCG | | | V5F | V5R |
| | AATCAACCCTGACCCCTCTCC | | | | |
| | TTCATAAATTATTCAGCTTCCT | | | | |
| | ACACTATTAAAGTTTACCACA | | | | |
| | ACCACCACCCCATCATACTCT | | | | |
| | TTCACCCACAGCACCAATCCT | | | | |
| | ACCTCCATCGCTAACCCCACT | | | | |
| | AAAACACTCACCAAGACCTCA | | | | |
| | ACCCCTGACCCCCATGCCTCA | | | | |
| | GGATACTCCTCAATAGC (SEQ | | | | |
| | ID NO: 545) | | | | |
| ND4 | GACTTCAAACTCTACTCCCAC | 202 | Human | HTS_ | HTS_ |
| | TAATAGCTTTTTGATGACTTCT | | | ND4_F | ND4_R |
| | AGCAAGCCTCGCTAACCTCGC | | | | |
| | CTTACCCCCCACTATTAACCT | | | | |
| | ACTGGGAGAACTCTCTGTGCT | | | | |
| | AGTAACCACGTTCTCCTGATC | | | | |
| | AAATATCACTCTCCTACTTAC | | | | |
| | AGGACTCAACATACTAGTCAC | | | | |
| | AGCCCTATACTCCCTCTACAT | | | | |
| | ATTTACCACAAC (SEQ ID NO: | | | | |
| | 546) | | | | |
| MT-TK | GGAGCAAACCACAGTTTCATG | 247 | Human | HTS_MT- | HTS_MT- |
| | CCCATCGTCCTAGAATTAATT | | | TK_F | TK_R |
| | CCCCTAAAAATCTTTGAAATA | | | | |
| | GGGCCCGTATTTACCCTATAG | | | | |
| | CACCCCCTCTACCCCCTCTAG | | | | |
| | AGCCCACTGTAAAGCTAACTT | | | | |
| | AGCATTAACCTTTTAAGTTAA | | | | |
| | AGATTAAGAGAACCAACACC | | | | |
| | TCTTTACAGTGAAATGCCCCA | | | | |
| | ACTAAATACTACCGTATGGCC | | | | |
| | CACCATAATTACCCCCATACT | | | | |
| | CCTTACACTATTCCTCA (SEQ | | | | |
| | ID NO: 547) | | | | |
| Mt-tk | GATCTAACCATAGCTTTATGC | 211 | Mouse | HTS_Mt- | HTS_Mt- |
| | CCATTGTCCTAGAAATGGTTC | | | tk_F | tk_R |
| | CACTAAAATATTTCGAAAACT | | | | |
| | GATCTGCTTCAATAATTTAAT | | | | |
| | TTCACTATGAAGCTAAGAGCG | | | | |
| | TTAACCTTTTAAGTTAAAGTT | | | | |
| | AGAGACCTTAAAATCTCCATA | | | | |
| | GTGATATGCCACAACTAGATA | | | | |
| | CATCAACATGATTTATCACAA | | | | |
| | TTATCTCATCAATAATTACCC | | | | |
| | T (SEQ ID NO: 548) | | | | |
| Nd1 | CTAGCCTATCAGTTTACTCCA | 236 | Mouse | HTS_ | HTS_ |
| | TTCTATGATCAGGATGAGCCT | | | Nd1_F | Nd1_R |
| | CAAACTCCAAATACTCACTAT | | | | |
| | TCGGAGCTTTACGAGCCGTAG | | | | |
| | CCCAAACAATTTCATATGAAG | | | | |
| | TAACCATAGCTATTATCCTTTT | | | | |
| | ATCAGTTCTATTAATAAATGG | | | | |
| | ATCCTACTCTCTACAAACACT | | | | |
| | TATTACAACCCAAGAACACAT | | | | |
| | ATGATTACTTCTGCCAGCCTG | | | | |
| | ACCCATAGCCATAATATGATT | | | | |
| | TATC (SEQ ID NO: 549) | | | | |
| JSK-ND1 | ACCATCGCTCTTCTACTATGA | 246 | Human | HTS_JSK- | HTS_JSK- |
| | ACCCCCCTCCCCATACCCAAC | | | ND1_F | ND1_R |
| | CCCCTGGTCAACCTCAACCTA | | | | |
| | GGCCTCCTATTTATTCTAGCC | | | | |
| | ACCTCTAGCCTAGCCGTTTAC | | | | |
| | TCAATCCTCTGATCAGGGTGA | | | | |
| | GCATCAAACTCAAACTACGCC | | | | |
| | CTGATCGGCGCACTGCGAGCA | | | | |
| | GTAGCCCAAACAATCTCATAT | | | | |
| | GAAGTCACCCTAGCCATCATT | | | | |
| | CTACTATCAACATTACTAATA | | | | |
| | AGTGGCTCCTTTAAC (SEQ ID | | | | |
| | NO: 550) | | | | |
| JSK-ND2 | GGGCCATTATCGAAGAATTCA | 231 | Human | HTS_JSK- | HTS_JSK- |
| | CAAAAAACAATAGCCTCATCA | | | ND2_F | ND2_R |
| | TCCCCACCATCATAGCCACCA | | | | |
| | TCACCCTCCTTAACCTTTACTT | | | | |
| | CTACCTACGCCTAATCTACTC | | | | |
| | CACCTCAATCACACTACTCCC | | | | |
| | CATATCTAACAACGTAAAAAT | | | | |
| | AAAATGACAGTTTGAACATAC | | | | |
| | AAAACCCACCCCATTCCTCCC | | | | |
| | CACACTCATCGCCCTTACCAC | | | | |
| | GCTACTCCTACCTATCTCCC | | | | |
| | (SEQ ID NO: 551) | | | | |
| JSK-ND4L | TCATAACCCTCAACACCCACT | 216 | Human | HTS_JSK- | HTS_JSK- |
| | CCCTCTTAGCCAATATTGTGC | | | ND4L_F | ND4LR |
| | CTATTGCCATACTAGTCTTTG | | | | |
| | CCGCCTGCGAAGCAGCGGTG | | | | |
| | GGCCTAGCCCTACTAGTCTCA | | | | |
| | ATCTCCAACACATATGGCCTA | | | | |
| | GACTACGTACATAACCTAAAC | | | | |
| | CTACTCCAATGCTAAAACTAA | | | | |
| | TCGTCCCAACAATTATATTAC | | | | |
| | TACCACTGACATGACTTTCCA | | | | |
| | AAAAACA (SEQ ID NO: 552) | | | | |
| JSK-ND4 | CTTATCCAGTGAACCACTATC | 222 | Human | HTS_JSK- | HTS_JSK- |
| | ACGAAAAAAACTCTACCTCTC | | | ND4_F | ND4_R |
| | TATACTAATCTCCCTACAAAT | | | | |
| | CTCCTTAATTATAACATTCAC | | | | |
| | AGCCACAGAACTAATCATATT | | | | |
| | TTATATCTTCTTCGAAACCAC | | | | |
| | ACTTATCCCCACCTTGGCTAT | | | | |
| | CATCACCCGATGAGGCAACCA | | | | |
| | GCCAGAACGCCTGAACGCAG | | | | |
| | GCACATACTTCCTATTCTACA | | | | |
| | CCCTAGTAGGCTC (SEQ ID | | | | |
| | NO: 553) | | | | |
| JSK-ND5 | CCTTCTTGCTCATCAGTTGAT | 231 | Human | HTS_JSK- | HTS_JSK- |
| | GATACGCCCGAGCAGATGCC | | | ND5_F | ND5_R |
| | AACACAGCAGCCATTCAAGC | | | | |
| | AATCCTATACAACCGTATCGG | | | | |
| | CGATATCGGTTTCATCCTCGC | | | | |
| | CTTAGCATGATTTATCCTACA | | | | |
| | CTCCAACTCATGAGACCCACA | | | | |
| | ACAAATAGCCCTTCTAAACGC | | | | |
| | TAATCCAAGCCTCACCCCACT | | | | |
| | ACTAGGCCTCCTCCTAGCAGC | | | | |
| | AGCAGGCAAATCAGCCCAATT | | | | |
| | AG (SEQ ID NO: 554) | | | | |
| JSK-ND52 | GCTATTACCTAAAACAATTTC | 230 | Human | HTS_JSK- | HTS_JSK- |
| | ACAGCACCAAATCTCCACCTC | | | ND52_F | ND52_R |
| | CATCATCACCTCAACCCAAAA | | | | |
| | AGGCATAATTAAACTTTACTT | | | | |
| | CCTCTCTTTCTTCTTCCCACTC | | | | |
| | ATCCTAACCCTACTCCTAATC | | | | |
| | ACATAACCTATTCCCCCGAGC | | | | |
| | AATCTCAATTACAATATATAC | | | | |
| | ACCAACAAACAATGTTCAACC | | | | |
| | AGTAACTACTACTAATCAACG | | | | |
| | CCCATAATCATACAAAGCC | | | | |
| | (SEQ ID NO: 555) | | | | |
| JSK-COX1 | CATCATAATCGGAGGCTTTGG | 213 | Human | HTS_JSK- | HTS_JSK- |
| | CAACTGACTAGTTCCCCTAAT | | | COX1_F | COX1_R |
| | AATCGGTGCCCCCGATATGGC | | | | |
| | GTTTCCCCGCATAAACAACAT | | | | |
| | AAGCTTCTGACTCTTACCTCC | | | | |
| | CTCTCTCCTACTCCTGCTCGCA | | | | |
| | TCTGCTATAGTGGAGGCCGGA | | | | |
| | GCAGGAACAGGTTGAACAGT | | | | |
| | CTACCCTCCCTTAGCAGGGAA | | | | |
| | CTACTCCCACCCTGGAGCCTC | | | | |
| | CGT (SEQ ID NO: 556) | | | | |
| JSK-COX2 | TGCCCTTTTCCTAACACTCAC | 228 | Human | HTS_JSK- | HTS_JSK- |
| | AACAAAACTAACTAATACTAA | | | COX2_F | COX2_R |
| | CATCTCAGACGCTCAGGAAAT | | | | |
| | AGAAACCGTCTGAACTATCCT | | | | |
| | GCCCGCCATCATCCTAGTCCT | | | | |
| | CATCGCCCTCCCATCCCTACG | | | | |
| | CATCCTTTACATAACAGACGA | | | | |
| | GGTCAACGATCCCTCCCTTAC | | | | |
| | CATCAAATCAATTGGCCACCA | | | | |
| | ATGGTACTGAACCTACGAGTA | | | | |
| | CACCGACTACGGCGGACT | | | | |
| | (SEQ ID NO: 557) | | | | |
| JSK-CYB | CGGGCGAGGCCTATATTACGG | 239 | Human | HTS_JSK- | HTS_JSK- |
| | ATCATTTCTCTACTCAGAAAC | | | CYB_F | CYB_R |
| | CTGAAACATCGGCATTATCCT | | | | |
| | CCTGCTTGCAACTATAGCAAC | | | | |
| | AGCCTTCATAGGCTATGTCCT | | | | |
| | CCCGTGAGGCCAAATATCATT | | | | |
| | CTGAGGGGCCACAGTAATTAC | | | | |
| | AAACTTACTATCCGCCATCCC | | | | |
| | ATACATTGGGACAGACCTAGT | | | | |
| | TCAATGAATCTGAGGAGGCTA | | | | |
| | CTCAGTAGACAGTCCCACCCT | | | | |
| | CACACGAT (SEQ ID NO: 558) | | | | |
| OT1 | ACAAAGGTTTGGTCCTGGCCT | 260 | Mouse | HTS_ | HTS_ |
| | TATAATTAATTAGAGGTAAAA | | | OT1_F | OT1_R |
| | TTACACATGCAAACCTCCATA | | | | |
| | GACCGGTGTAAAATCCCTTAA | | | | |
| | ACATTTACTTAAAATTTAAGG | | | | |
| | AGAGGGTATCAAGCACATTA | | | | |
| | AAATAGCTTAAGACACCTTGC | | | | |
| | CTAGCCACACCCCCACGGGAC | | | | |
| | TCAGCAGTGATAAATATTAAG | | | | |
| | CAATAAACGAAAGTTTGACTA | | | | |
| | AGTTATACCTCTTAGGGTTGG | | | | |
| | TAAATTTCGTGCCAGCCACCG | | | | |
| | CGGTCATAC (SEQ ID | | | | |
| | NO: 559) | | | | |
| OT2 | GTCATTTATAATACACGACAG | 249 | Mouse | HTS_ | HTS_ |
| | CTAAGACCCAAACTGGGATTA | | | OT2_F | OT2_R |
| | GATACCCCACTATGCTTAGCC | | | | |
| | ATAAACCTAAATAATTAAATT | | | | |
| | TAACAAAACTATTTGCCAGAG | | | | |
| | AACTACTAGCCATAGCTTAAA | | | | |
| | ACTCAAAGGACTTGGCGGTAC | | | | |
| | TTTATATCCATCTAGAGGAGC | | | | |
| | CTGTTCTATAATCGATAAACC | | | | |
| | CCGCTCTACCTCACCATCTCTT | | | | |
| | GCTAATTCAGCCTATATACCG | | | | |
| | CCATCTTCAGCAAACCC (SEQ | | | | |
| | ID NO: 560) | | | | |
| OT3 | GCCTACACCCAGAAGATTTCA | 261 | Mouse | HTS_ | HTS_ |
| | TGACCAATGAACACTCTGAAC | | | OT3_F | OT3_R |
| | TAATCCTAGCCCTAGCCCTAC | | | | |
| | ACAAATATAATTATACTATTA | | | | |
| | TATAAATCAAAACATTTATCC | | | | |
| | TACTAAAAGTATTGGAGAAA | | | | |
| | GAAATTCGTACATCTAGGAGC | | | | |
| | TATAGAACTAGTACCGCAAGG | | | | |
| | GAAAGATGAAAGACTAATTA | | | | |
| | AAAGTAAGAACAAGCAAAGA | | | | |
| | TTAAACCTTGTACCTTTTGCAT | | | | |
| | AATGAACTAACTAGAAAACTT | | | | |
| | CTAACTAAAAG (SEQ ID NO: | | | | |
| | 561) | | | | |
| OT4 | CTTTAATCAGTGAAATTGACC | 278 | Mouse | HTS_ | HTS_ |
| | TTTCAGTGAAGAGGCTGAAAT | | | OT4_F | OT4_R |
| | ATAATAATAAGACGAGAAGA | | | | |
| | CCCTATGGAGCTTAAATTATA | | | | |
| | TAACTTATCTATTTAATTTATT | | | | |
| | AAACCTAATGGCCCAAAAACT | | | | |
| | ATAGTATAAGTTTGAAATTTC | | | | |
| | GGTTGGGGTGACCTCGGAGA | | | | |
| | ATAAAAAATCCTCCGAATGAT | | | | |
| | TATAACCTAGACTTACAAGTC | | | | |
| | AAAGTAAAATCAACATATCTT | | | | |
| | ATTGACCCAGATATATTTTGA | | | | |
| | TCAACGGACCAAGTTACCCTA | | | | |
| | GGGATA (SEQ ID NO: 562) | | | | |
| OT5 | ACAAACACTTATTACAACCCA | 286 | Mouse | HTS_ | HTS_ |
| | AGAACACATATGATTACTTCT | | | OT5_F | OT5_R |
| | GCCAGCCTGACCCATAGCCAT | | | | |
| | AATATGATTTATCTCAACCCT | | | | |
| | AGCAGAAACAAACCGGGCCC | | | | |
| | CCTTCGACCTGACAGAAGGAG | | | | |
| | AATCAGAATTAGTATCAGGGT | | | | |
| | TTAACGTAGAATACGCAGCCG | | | | |
| | GCCCATTCGCGTTATTCTTTAT | | | | |
| | AGCAGAGTACACTAACATTAT | | | | |
| | TCTAATAAACGCCCTAACAAC | | | | |
| | TATTATCTTCCTAGGACCCCT | | | | |
| | ATACTATATCAATTTACCAGA | | | | |
| | ACTCTACTCAACT (SEQ ID NO: | | | | |
| | 563) | | | | |
| OT6 | CAACTGTCTAATTATAGCAAC | 265 | Mouse | HTS_OT | HTS_O |
| | ACTCATAGCAATAATAGCTCT | | | 6_F | T6_R |
| | ACTAAACCTATTCTTTTATACT | | | | |
| | CGCCTAATTTATTCCACTTCA | | | | |
| | CTAACAATATTTCCAACCAAC | | | | |
| | AATAACTCAAAAATAATAACT | | | | |
| | CACCAAACAAAAACTAAACC | | | | |
| | CAACCTAATATTTTCCACCCT | | | | |
| | AGCTATCATAAGCACAATAAC | | | | |
| | CCTACCCCTAGCCCCCCAACT | | | | |
| | AATTACCTAGAAGTTTAGGAT | | | | |
| | ATACTAGTCCGCGAGCCTTCA | | | | |
| | AAGCCCTAAGAAA (SEQ ID | | | | |
| | NO: 564) | | | | |
| OT7 | TAATATAAGTTTTTGACTCCT | 264 | Mouse | HTS_ | HTS_ |
| | ACCACCATCATTTCTCCTTCTC | | | OT7_F | OT7_R |
| | CTAGCATCATCAATAGTAGAA | | | | |
| | GCAGGAGCAGGAACAGGATG | | | | |
| | AACAGTCTACCCACCTCTAGC | | | | |
| | CGGAAATCTAGCCCATGCAGG | | | | |
| | AGCATCAGTAGACCTAACAAT | | | | |
| | TTTCTCCCTTCATTTAGCTGGA | | | | |
| | GTGTCATCTATTTTAGGTGCA | | | | |
| | ATTAATTTTATTACCACTATTA | | | | |
| | TCAACATGAAACCCCCAGCCA | | | | |
| | TAACACAGTATCAAACTCCAC | | | | |
| | TATTTGTCTG (SEQ ID NO: | | | | |
| | 565) | | | | |
| OT8 | CTTCAACAAATTTAGAATGAC | 272 | Mouse | HTS_ | HTS_ |
| | TTCATGGCTGCCCTCCACCAT | | | OT8_F | OT8_R |
| | ATCACACATTCGAGGAACCAA | | | | |
| | CCTATGTAAAAGTAAAATAAG | | | | |
| | AAAGGAAGGAATCGAACCCC | | | | |
| | CTAAAATTGGTTTCAAGCCAA | | | | |
| | TCTCATATCCTATATGTCTTTC | | | | |
| | TCAATAAGATATTAGTAAAAT | | | | |
| | CAATTACATAACTTTGTCAAA | | | | |
| | GTTAAATTATAGATCAATAAT | | | | |
| | CTATATATCTTATATGGCCTA | | | | |
| | CCCATTCCAACTTGGTCTACA | | | | |
| | AGACGCCACATCCCCTATTA | | | | |
| | (SEQ ID NO: 566) | | | | |
| OT9 | TCATCATAGCCTTATAGAAGG | 241 | Mouse | HTS_ | HTS_ |
| | TAAACGAAACCACATAAATC | | | OT9_F | OT9_R |
| | AAGCCCTACTAATTACCATTA | | | | |
| | TACTAGGACTTTACTTCACCA | | | | |
| | TCCTCCAAGCTTCAGAATACT | | | | |
| | TTGAAACATCATTCTCCATTT | | | | |
| | CAGATGGTATCTATGGTTCTA | | | | |
| | CATTCTTCATGGCTACTGGAT | | | | |
| | TCCATGGACTCCATGTAATTA | | | | |
| | TTGGATCAACATTCCTTATTG | | | | |
| | TTTGCCTACTACGACAACTAA | | | | |
| | AATTTCACTTC (SEQ ID NO: | | | | |
| | 567) | | | | |
| OT10 | ACCATAGCCTTCTCACTATCA | 275 | Mouse | HTS_ | HTS_ |
| | CTTCTAGGGACACTTATATTT | | | OT10_F | OT10_R |
| | CGCTCTCACCTAATATCCACA | | | | |
| | TTACTATGCCTGGAAGGCATA | | | | |
| | GTATTATCCTTATTTATTATAA | | | | |
| | CTTCAGTAACTTCCCTAAACT | | | | |
| | CCAACTCCATAAGCTCCATAC | | | | |
| | CAATCCCCATCACCATCTTAG | | | | |
| | TTTTCGCAGCCTGCGAAGCAG | | | | |
| | CTGTAGGACTAGCCCTACTAG | | | | |
| | TAAAAGTTTCAAACACGTACG | | | | |
| | GAACAGATTACGTCCAAAATC | | | | |
| | TCAACCTACTACAATGCTAAA | | | | |
| | A (SEQ ID NO: 568) | | | | |
| OT11 | CCTTAGACGCTTCATGATCTA | 254 | Mouse | HTS_ | HTS_ |
| | ACAACTTACTATGGTTGGCAT | | | OT11_F | OT11_R |
| | GCATAATAGCATTTCTTATTA | | | | |
| | AAATACCATTATATGGAGTTC | | | | |
| | ACCTATGACTACCAAAAGCCC | | | | |
| | ATGTTGAAGCTCCAATTGCTG | | | | |
| | GGTCAATAATTCTAGCAGCTA | | | | |
| | TTCTTCTAAAATTAGGTAGTT | | | | |
| | ACGGAATAATTCGCATCTCCA | | | | |
| | TTATTCTAGACCCACTAACAA | | | | |
| | AATATATAGCATACCCCTTCA | | | | |
| | TCCTTCTCTCCCTATGAGGAA | | | | |
| | TA (SEQ ID NO: 569) | | | | |
| OT12 | CTTTTCATTGGCTGAGAAGGG | 274 | Mouse | HTS_ | HTS_ |
| | GTGGGAATTATATCTTTCCTA | | | OT12_F | OT12_R |
| | CTAATTGGATGATGGTACGGA | | | | |
| | CGAACAGACGCAAATACTGC | | | | |
| | AGCCCTACAAGCAATCCTCTA | | | | |
| | TAACCGCATCGGAGACATCGG | | | | |
| | ATTCATTTTAGCTATAGTTTG | | | | |
| | ATTTTCCCTAAACATAAACTC | | | | |
| | ATGAGAACTTCAACAGATTAT | | | | |
| | ATTCTCCAACAACAACGACAA | | | | |
| | TCTAATTCCACTTATAGGCCT | | | | |
| | ATTAATCGCAGCTACAGGAAA | | | | |
| | ATCAGCACAATTTGGCCTCCA | | | | |
| | CC (SEQ ID NO: 570) | | | | |
| HBB | CCTAGGGTTGGCCAATCTACT | 225 | Human | HTS_ | HTS_ |
| | CCCAGGAGCAGGGAGGGCAG | | | HBB_F | HBB_R |
| | GAGCCAGGGCTGGGCATAGA | | | | |
| | AGTCAGGGCAGAGCCATCTAT | | | | |
| | TGCTTACATTTGCTTCTGACA | | | | |
| | CAACTGTGTTCACTAGCAACC | | | | |
| | TCAAACAGACACCATGGTGCA | | | | |
| | TCTGACTCCTGAGGAGAAGTC | | | | |
| | TGCCGTTACTGCCCTGTGGGG | | | | |
| | CAAGGTAAACCAGCTTTACAC | | | | |
| | TCCCTCACACTGATCAC (SEQ | | | | |
| | ID NO: 571) | | | | |
| COL5A1 | TGGTGCTGTGGGTGGGGTCCT | 223 | Human | HTS_ | HTS_ |
| | CTCTATTTTGTCCTCTGTAGGT | | | COL5A1_F | OCL5A1_R |
| | GCCTTTTCCTCCGTACCCCTCT | | | | |
| | TCAGACCAGGCCTCTGAGATT | | | | |
| | TGCCTCCTACTCAGGACACCC | | | | |
| | AGGGTTGGGTGGAGGCCACG | | | | |
| | GCTCTGCCCACACCCCCCTGC | | | | |
| | CCCTTGGCCAACACCCTGTCT | | | | |
| | TCGCCTGTGGCTCTCCTGCAC | | | | |
| | CCTGGGGCCTGGGCTCCAGAA | | | | |
| | TCAGCCTCCCTT (SEQ ID NO: | | | | |
| | 572) | | | | |
| DCAF82L | ATGGTTTCACAACAGCACATG | 233 | Human | HTS_ | HTS_ |
| | GTGAAAAGGTTAAAAAAAAA | | | DCAF8L2_F | CDAF8L2_R |
| | AAGTCACCCTATACATTGTCA | | | | |
| | ATGAAATTCATATGAGGAGCT | | | | |
| | ATAACCTGACACTCCTACTCT | | | | |
| | CCCCATCAGGGAGGAATGAG | | | | |
| | TGGAGGCCTAGACAGCGACC | | | | |
| | AGAACTCCCAACCCCAACCAG | | | | |
| | GAGTAACCAGGAACATCCCTC | | | | |
| | ACTCAGGAGTCAATGGGGGC | | | | |
| | CAGGGATGGAACACTCTTTTA | | | | |
| | CACTCA (SEQ ID NO: 573) | | | | |
| EMILIN2 | TGAAGTCTGGAGACTCACTGA | 226 | Human | HTS_ | HTS_ |
| | ATTTGTTCTGGGTATGGGGGA | | | EMILIN2_F | EMILIN2_R |
| | GGTTACATGGGTTTAATGGTT | | | | |
| | TTTAAATTTATTTGGGGGGAA | | | | |
| | TAAGGGTTGTCTTTGGGTACA | | | | |
| | CTACAGCGATGGCTATTGAGG | | | | |
| | AGTATCCTGAGGCATGGGGGT | | | | |
| | CAGGGGTTGAGGTCTTGGTAA | | | | |
| | GTGTTTTAGTGGGGTTAGCGA | | | | |
| | TTCTGGTACAGTGGCTTGTGC | | | | |
| | CTGTAATCCCAGCACT (SEQ | | | | |
| | ID NO: 574) | | | | |
| TRAM1L1 | TCTAGCTTATATTCACTAATG | 281 | Human | HTS_ | HTS_ |
| | GGAAAACGTATTCTAATAAAC | | | TRAM1L1_F | TRAMIL1_ |
| | TAGCACTGACTCATTACATGA | | | | R |
| | ATAGATCTAAGCCATCAAGTG | | | | |
| | ACAGACAGAAAATTATGTGCT | | | | |
| | GTTGTGAAAGACAAGGAAGG | | | | |
| | GCAGGAGAAGATAGTGAGAA | | | | |
| | GTCAGTGAGGCTCCAAGCAA | | | | |
| | AATTATGCTGCATTTGATATA | | | | |
| | TTTCTCCACATGTAAATACAC | | | | |
| | AACAGGTGTAGCTGAAACTTG | | | | |
| | CTTGCTAGTCATGTAAAACAT | | | | |
| | TGCACTATGAAACTTTACTTA | | | | |
| | CAATTAGAGAC (SEQ ID NO: | | | | |
| | 575) | | | | |

*ND1 (GNN)n-rich site 1
**COX1 (GNN)n-rich site 1
#COX1 (GNN)n-rich site 2
##COX2 (GNN)n-rich site 1
^{∧}ND6 (GNN)n-rich site 1

TABLE 6
HTS Primers

| | | SEQ | |
|---|---|---|---|
| | | ID | |
| Name | Sequence | NO: | Length |
| HTS_ATP8_F | ACACTCTTTCCCTACACGACGCTCTTCCGA | 576 | 59 |
| | TCTNNNNCTTTACAGTGAAATGCCCCAAC | | |
| HTS_ATP8_R | TGGAGTTCAGACGTGTGCTCTTCCGATCT | 577 | 48 |
| | GGGGGCAATGAATGAAGCG | | |
| HTS_ND51_F | ACACTCTTTCCCTACACGACGCTCTTCCGA | 578 | 56 |
| | TCTNNNNCGGGTCCATCATCCACAAC | | |
| HTS_ND51_R | TGGAGTTCAGACGTGTGCTCTTCCGATCT | 579 | 52 |
| | AGAGTAATAGATAGGGCTCAGGC | | |
| HTS_ND62_F | ACACTCTTTCCCTACACGACGCTCTTCCGA | 580 | 59 |
| | TCTNNNNAAAGTTTACCACAACCACCACC | | |
| HTS_ND62_R | TGGAGTTCAGACGTGTGCTCTTCCGATCTT | 581 | 55 |
| | GGGGGAGGTTATATGGGTTTAATAG | | |
| HTS_COX1_F | ACACTCTTTCCCTACACGACGCTCTTCCGA | 582 | 57 |
| | TCTNNNNCCTACTCCTGCTCGCATCTG | | |
| HTS_COX1_R | TGGAGTTCAGACGTGTGCTCTTCCGATCTT | 583 | 50 |
| | GGTATTGGGTTATGGCAGGG | | |
| HTS_V1_F | ACACTCTTTCCCTACACGACGCTCTTCCGA | 584 | 61 |
| | TCTNNNNGGCTATATACAACTACGCAAAG | | |
| | GC | | |
| HTS_V1_R | TGGAGTTCAGACGTGTGCTCTTCCGATCT | 585 | 54 |
| | ATAGGAGGCCTAGGTTGAGGTTGAC | | |
| HTS_V2_F | ACACTCTTTCCCTACACGACGCTCTTCCGA | 586 | 60 |
| | TCTNNNNAATCGGAGGCTTTGGCAACTGA | | |
| | C | | |
| HTS_V2_R | TGGAGTTCAGACGTGTGCTCTTCCGATCT | 587 | 53 |
| | GGTTAGGTCTACGGAGGCTCCAGG | | |
| HTS_V3_F | ACACTCTTTCCCTACACGACGCTCTTCCGA | 588 | 60 |
| | TCTNNNNATACCAAACGCCCCTCTTCGTC | | |
| | T | | |
| HTS_V3_R | TGGAGTTCAGACGTGTGCTCTTCCGATCT | 589 | 55 |
| | CCGAAGCCTGGTAGGATAAGAATATA | | |
| HTS_V4_F | ACACTCTTTCCCTACACGACGCTCTTCCGA | 590 | 62 |
| | TCTNNNNGCATCCTTTACATAACAGACGA | | |
| | GGT | | |
| HTS_V4_R | TGGAGTTCAGACGTGTGCTCTTCCGATCTT | 591 | 57 |
| | TATACGAATGGGGGCTTCAATCGGGAG | | |
| HTS_V5_F | ACACTCTTTCCCTACACGACGCTCTTCCGA | 592 | 63 |
| | TCTNNNNCCCATAATCATACAAAGCCCCC | | |
| | GCAC | | |
| HTS_V5_R | TGGAGTTCAGACGTGTGCTCTTCCGATCT | 593 | 53 |
| | GCTATTGAGGAGTATCCTGAGGCA | | |
| HTS_ND4_F | ACACTCTTTCCCTACACGACGCTCTTCCGA | 594 | 64 |
| | TCTNNNNGACTTCAAACTCTACTCCCACT | | |
| | AATAG | | |
| HTS_ND4_R | TGGAGTTCAGACGTGTGCTCTTCCGATCT | 595 | 53 |
| | GTTGTGGTAAATATGTAGAGGGAG | | |
| HTS_MT-TK_F | ACACTCTTTCCCTACACGACGCTCTTCCGA | 596 | 58 |
| | TCTNNNNGGAGCAAACCACAGTTTCATG | | |
| HTS_MT-TK_R | TGGAGTTCAGACGTGTGCTCTTCCGATCTT | 597 | 54 |
| | GAGGAATAGTGTAAGGAGTATGGG | | |
| HTS_Mt-tk_F | ACACTCTTTCCCTACACGACGCTCTTCCGA | 598 | 63 |
| | TCTNNNNGATCTAACCATAGCTTTATGCC | | |
| | CATT | | |
| HTS_Mt-tk_R | TGGAGTTCAGACGTGTGCTCTTCCGATCT | 599 | 56 |
| | AGGGTAATTATTGATGAGATAATTGTG | | |
| HTS_Nd1_F | ACACTCTTTCCCTACACGACGCTCTTCCGA | 600 | 67 |
| | TCTNNNNCTAGCCTATCAGTTTACTCCATT | | |
| | CTATGAT | | |
| HTS_Nd1_R | TGGAGTTCAGACGTGTGCTCTTCCGATCT | 601 | 59 |
| | GATAAATCATATTATGGCTATGGGTCAGG | | |
| | C | | |
| HTS_JSK- | ACACTCTTTCCCTACACGACGCTCTTCCGA | 602 | 63 |
| ND1_F | TCTNNNNACCATCGCTCTTCTACTATGAA | | |
| | CCCC | | |
| HTS_JSK- | TGGAGTTCAGACGTGTGCTCTTCCGATCT | 603 | 57 |
| ND1_R | GTTAAAGGAGCCACTTATTAGTAATGTT | | |
| HTS_JSK- | ACACTCTTTCCCTACACGACGCTCTTCCGA | 604 | 63 |
| ND2_F | TCTNNNNGGGCCATTATCGAAGAATTCAC | | |
| | AAAA | | |
| HTS_JSK- | TGGAGTTCAGACGTGTGCTCTTCCGATCT | 605 | 58 |
| ND2_R | GGGAGATAGGTAGGAGTAGCGTGGTAAG | | |
| | G | | |
| HTS_JSK- | ACACTCTTTCCCTACACGACGCTCTTCCGA | 606 | 63 |
| ND4L_F | TCTNNNNTCATAACCCTCAACACCCACTC | | |
| | CCTC | | |
| HTS_JSK- | TGGAGTTCAGACGTGTGCTCTTCCGATCTT | 607 | 57 |
| ND4L_R | GTTTTTTGGAAAGTCATGTCAGTGGTA | | |
| HTS_JSK- | ACACTCTTTCCCTACACGACGCTCTTCCGA | 608 | 64 |
| ND4_F | TCTNNNNCTTATCCAGTGAACCACTATCA | | |
| | CGAAA | | |
| HTS_JSK- | TGGAGTTCAGACGTGTGCTCTTCCGATCT | 609 | 56 |
| ND4_R | GAGCCTACTAGGGTGTAGAATAGGAAG | | |
| HTS_JSK- | ACACTCTTTCCCTACACGACGCTCTTCCGA | 610 | 67 |
| ND5_F | TCTNNNNCCTTCTTGCTCATCAGTTGATGA | | |
| | TACGCCC | | |
| HTS_JSK- | TGGAGTTCAGACGTGTGCTCTTCCGATCT | 611 | 55 |
| ND5_R | CTAATTGGGCTGATTTGCCTGCTGCT | | |
| HTS_JSK- | ACACTCTTTCCCTACACGACGCTCTTCCGA | 612 | 66 |
| ND52_F | TCTNNNNGCTATTACCTAAAACAATTTCA | | |
| | CAGCACC | | |
| HTS_JSK- | TGGAGTTCAGACGTGTGCTCTTCCGATCT | 613 | 59 |
| ND52_R | GGCTTTGTATGATTATGGGCGTTGATTAG | | |
| | T | | |
| HTS_JSK- | ACACTCTTTCCCTACACGACGCTCTTCCGA | 614 | 63 |
| COX1_F | TCTNNNNCATCATAATCGGAGGCTTTGGC | | |
| | AACT | | |
| HTS_JSK- | TGGAGTTCAGACGTGTGCTCTTCCGATCT | 615 | 54 |
| COX1_R | ACGGAGGCTCCAGGGTGGGAGTAGT | | |
| HTS_JSK- | ACACTCTTTCCCTACACGACGCTCTTCCGA | 616 | 65 |
| COX2_F | TCTNNNNTGCCCTTTTCCTAACACTCACA | | |
| | ACAAAA | | |
| HTS_JSK- | TGGAGTTCAGACGTGTGCTCTTCCGATCT | 617 | 60 |
| COX2_R | AGTCCGCCGTAGTCGGTGTACTCGTAGGT | | |
| | TC | | |
| HTS_JSK- | ACACTCTTTCCCTACACGACGCTCTTCCGA | 618 | 64 |
| CYB_F | TCTNNNNCGGGCGAGGCCTATATTACGGA | | |
| | TCATT | | |
| HTS_JSK- | TGGAGTTCAGACGTGTGCTCTTCCGATCT | 619 | 57 |
| CYB_R | ATCGTGTGAGGGTGGGACTGTCTACTGA | | |
| HTS_OT1_F | ACACTCTTTCCCTACACGACGCTCTTCCGA | 620 | 65 |
| | TCTNNNNACAAAGGTTTGGTCCTGGCCTT | | |
| | ATAATT | | |
| HTS_OT1_R | TGGAGTTCAGACGTGTGCTCTTCCGATCT | 621 | 56 |
| | GTATGACCGCGGTGGCTGGCACGAAAT | | |
| HTS_OT2_F | ACACTCTTTCCCTACACGACGCTCTTCCGA | 622 | 67 |
| | TCTNNNNGTCATTTATAATACACGACAGC | | |
| | TAAGACCC | | |
| HTS_OT2_R | TGGAGTTCAGACGTGTGCTCTTCCGATCT | 623 | 57 |
| | GGGTTTGCTGAAGATGGCGGTATATAGG | | |
| HTS_OT3_F | ACACTCTTTCCCTACACGACGCTCTTCCGA | 624 | 66 |
| | TCTNNNNGCCTACACCCAGAAGATTTCAT | | |
| | GACCAAT | | |
| HTS_OT3_R | TGGAGTTCAGACGTGTGCTCTTCCGATCT | 625 | 59 |
| | CTTTTAGTTAGAAGTTTTCTAGTTAGTTCA | | |
| HTS_OT4_F | ACACTCTTTCCCTACACGACGCTCTTCCGA | 626 | 64 |
| | TCTNNNNCTTTAATCAGTGAAATTGACCT | | |
| | TTCAG | | |
| HTS_OT4_R | TGGAGTTCAGACGTGTGCTCTTCCGATCTT | 627 | 57 |
| | ATCCCTAGGGTAACTTGGTCCGTTGAT | | |
| HTS_OT5_F | ACACTCTTTCCCTACACGACGCTCTTCCGA | 628 | 67 |
| | TCTNNNNACAAACACTTATTACAACCCAA | | |
| | GAACACAT | | |
| HTS_OT5_R | TGGAGTTCAGACGTGTGCTCTTCCGATCT | 629 | 57 |
| | AGTTGAGTAGAGTTCTGGTAAATTGATA | | |
| HTS_OT6_F | ACACTCTTTCCCTACACGACGCTCTTCCGA | 630 | 67 |
| | TCTNNNNCAACTGTCTAATTATAGCAACA | | |
| | CTCATAGC | | |
| HTS_OT6_R | TGGAGTTCAGACGTGTGCTCTTCCGATCTT | 631 | 58 |
| | TTCTTAGGGCTTTGAAGGCTCGCGGACT | | |
| HTS_OT7_F | ACACTCTTTCCCTACACGACGCTCTTCCGA | 632 | 67 |
| | TCTNNNNTAATATAAGTTTTTGACTCCTAC | | |
| | CACCATC | | |
| HTS_OT7_R | TGGAGTTCAGACGTGTGCTCTTCCGATCT | 633 | 59 |
| | CAGACAAATAGTGGAGTTTGATACTGTGT | | |
| | T | | |
| HTS_OT8_F | ACACTCTTTCCCTACACGACGCTCTTCCGA | 634 | 66 |
| | TCTNNNNCTTCAACAAATTTAGAATGACT | | |
| | TCATGGC | | |
| HTS_OT8_R | TGGAGTTCAGACGTGTGCTCTTCCGATCTT | 635 | 59 |
| | AATAGGGGATGTGGCGTCTTGTAGACCAA | | |
| HTS_OT9_F | ACACTCTTTCCCTACACGACGCTCTTCCGA | 636 | 67 |
| | TCTNNNNTCATCATAGCCTTATAGAAGGT | | |
| | AAACGAAA | | |
| HTS_OT9_R | TGGAGTTCAGACGTGTGCTCTTCCGATCT | 637 | 59 |
| | GAAGTGAAATTTTAGTTGTCGTAGTAGGC | | |
| | A | | |
| HTS_OT10_F | ACACTCTTTCCCTACACGACGCTCTTCCGA | 638 | 66 |
| | TCTNNNNACCATAGCCTTCTCACTATCAC | | |
| | TTCTAGG | | |
| HTS_OT10_R | TGGAGTTCAGACGTGTGCTCTTCCGATCTT | 639 | 60 |
| | TTTAGCATTGTAGTAGGTTGAGATTTTGG | | |
| | A | | |
| HTS_OT11_F | ACACTCTTTCCCTACACGACGCTCTTCCGA | 640 | 67 |
| | TCTNNNNCCTTAGACGCTTCATGATCTAA | | |
| | CAACTTAC | | |
| HTS_OT11_R | TGGAGTTCAGACGTGTGCTCTTCCGATCTT | 641 | 58 |
| | ATTCCTCATAGGGAGAGAAGGATGAAGG | | |
| HTS_OT12_F | ACACTCTTTCCCTACACGACGCTCTTCCGA | 642 | 66 |
| | TCTNNNNCTTTTCATTGGCTGAGAAGGGG | | |
| | TGGGAAT | | |
| HTS_OT12_R | TGGAGTTCAGACGTGTGCTCTTCCGATCT | 643 | 59 |
| | GGTGGAGGCCAAATTGTGCTGATTTTCCT | | |
| | G | | |
| HTS_HBB_F | ACACTCTTTCCCTACACGACGCTCTTCCGA | 644 | 66 |
| | TCTNNNNGTGATCAGTGTGAGGGAGTGTA | | |
| | AAGCTGG | | |
| HTS_HBB_R | TGGAGTTCAGACGTGTGCTCTTCCGATCT | 645 | 53 |
| | CCTAGGGTTGGCCAATCTACTCCC | | |
| HTS_COL5A1_F | ACACTCTTTCCCTACACGACGCTCTTCCGA | 646 | 67 |
| | TCTNNNNTGGTGCTGTGGGTGGGGTCCTC | | |
| | TCTATTTT | | |
| HTS_COL5A1_R | TGGAGTTCAGACGTGTGCTCTTCCGATCT | 647 | 57 |
| | AAGGGAGGCTGATTCTGGAGCCCAGGCC | | |
| HTS_DCAF8L2_ | ACACTCTTTCCCTACACGACGCTCTTCCGA | 648 | 69 |
| F | TCTNNNNATGGTTTCACAACAGCACATGG | | |
| | TGAAAAGGTT | | |
| HTS_DCAF8L2_ | TGGAGTTCAGACGTGTGCTCTTCCGATCTT | 649 | 59 |
| R | GAGTGTAAAAGAGTGTTCCATCCCTGGCC | | |
| HTS_EMILIN2_ | ACACTCTTTCCCTACACGACGCTCTTCCGA | 650 | 67 |
| F | TCTNNNNTGAAGTCTGGAGACTCACTGAA | | |
| | TTTGTTCT | | |
| HTS_EMILIN2_ | TGGAGTTCAGACGTGTGCTCTTCCGATCT | 651 | 58 |
| R | AGTGCTGGGATTACAGGCACAAGCCACTG | | |
| HTS_TRAM1L1_ | ACACTCTTTCCCTACACGACGCTCTTCCGA | 652 | 69 |
| F | TCTNNNNTCTAGCTTATATTCACTAATGG | | |
| | GAAAACGTAT | | |
| HTS_TRAM1L1_ | TGGAGTTCAGACGTGTGCTCTTCCGATCT | 653 | 61 |
| R | GTCTCTAATTGTAAGTAAAGTTTCATAGT | | |
| | GCA | | |

TABLE 7
Mitochondrial ZFs

| | | | | Target | | | | | | | | | |
|---|---|---|---|---|---|---|---|---|---|---|---|---|---|
| | | | | DNA | | | | | | | Target | | |
| | Gene | | | Sequence | | | | | | | DNA | DddA | Archi- |
| Name | target | Species | Amplicon | (5′ to 3′) | ZF1 | ZF2 | ZF3 | ZF4 | ZF5 | ZF6 | strand | split | tecture |
| R8-ATP8 | ATP8 | Human | ATP8 | AGGTAG | TSG | QSG | RSD | RND | RSD | — | LB | DddA^{N} | Canonical |
| | | | | GTGGTA | SLS | SLT | ALS | NRI | HLT | | | | |
| | | | | GTT | R | R | Q | T | Q | | | | |
| | | | | (SEQ ID | (SEQ | (SEQ | (SEQ | | | | | | |
| | | | | NO: 654) | ID | ID | ID | | | | | | |
| | | | | | NO: | NO: | NO: | | | | | | |
| | | | | | 889) | 881) | 886) | | | | | | |
| 4-ATP8 | ATP8 | Human | ATP8 | CACCAA | QAS | TSH | ERS | QSG | SKK | — | RT | DddA^{C} | Canonical |
| | | | | AGCCCA | NLI | SLT | HLR | NLT | ALT | | | | |
| | | | | TAA | S | E | E | E | E | | | | |
| | | | | (SEQ ID | (SEQ | (SEQ | (SEQ | (SEQ | (SEQ | | | | |
| | | | | NO: 655) | ID | ID | ID | ID | ID | | | | |
| | | | | | NO: | NO: | NO: | NO: | NO: | | | | |
| | | | | | 801) | 773) | 762) | 769) | 770) | | | | |
| 10-ATP8 | ATP8 | Human | ATP8 | AGCCCA | QASN | QRAN | QASN | TSHS | ERSH | — | RT | DddA^{C} | Canonical |
| | | | | TAAAAA | LIS | LRA | LIS | LTE | LRE | | | | |
| | | | | TAA | (SEQ | (SEQ | (SEQ | (SEQ | (SEQ | | | | |
| | | | | (SEQ ID | ID | ID | ID | ID | ID | | | | |
| | | | | NO: 656) | NO: | NO: | NO: | NO: | NO: | | | | |
| | | | | | 801) | 753) | 801) | 773) | 762) | | | | |
| 9-ND51 | ND5 | Human | ND51 | TTGAAG | QSS | RSD | QA | RKD | RKD | — | LB | DddA^{C} | Canonical |
| | | | | TGAGAG | SLV | NLV | GHL | NLK | ALR | | | | |
| | | | | GTA | R | R | AS | N | G | | | | |
| | | | | (SEQ ID | (SEQ | (SEQ | (SEQ | (SEQ | (SEQ | | | | |
| | | | | NO: 657) | ID | ID | ID | ID | ID | | | | |
| | | | | | NO: | NO: | NO: | NO: | NO: | | | | |
| | | | | | 797) | 787) | 809) | 755) | 815) | | | | |
| 12-ND51 | ND5 | Human | ND51 | AAGTGA | RSD | QSS | RSD | QA | RKD | — | LB | DddA^{C} | Canonical |
| | | | | GAGGTA | HLT | SLV | NLV | GHL | NLK | | | | |
| | | | | TGG | T | R | R | AS | N | | | | |
| | | | | (SEQ ID | (SEQ | (SEQ | (SEQ | (SEQ | (SEQ | | | | |
| | | | | NO: 658) | ID | ID | ID | ID | ID | | | | |
| | | | | | NO: | NO: | NO: | NO: | NO: | | | | |
| | | | | | 811) | 797) | 787) | 809) | 755) | | | | |
| R13-ND51 | ND5 | Human | ND51 | CCATTG | RSD | DRS | QSG | RSD | QK | — | RT | DddA^{N} | Canonical |
| | | | | GCAGCC | NLS | DLS | DLT | SLS | ATR | | | | |
| | | | | TAG | T | R | R | A | IT | | | | |
| | | | | (SEQ ID | (SEQ | | | | | | | | |
| | | | | NO: 659) | ID | | | | | | | | |
| | | | | | NO: | | | | | | | | |
| | | | | | 887) | | | | | | | | |
| R8-4i-ATP8 | ATP8 | Human | ATP8 | TAGGTG | TSG | QSG | RSD | RND | — | — | LB | DddA^{N} | Canonical |
| | | | | GTAGTT | SLS | SLT | ALS | NRI | | | | | |
| | | | | (SEQ ID | R | R | Q | T | | | | | |
| | | | | NO: 660) | (SEQ | (SEQ | (SEQ | | | | | | |
| | | | | | ID | ID | ID | | | | | | |
| | | | | | NO: | NO: | NO: | | | | | | |
| | | | | | 889) | 881) | 886) | | | | | | |
| R8-4ii-ATP8 | ATP8 | Human | ATP8 | AGGTAG | QSG | RSD | RND | RSD | — | — | LB | DddA^{N} | Canonical |
| | | | | GTGGTA | SLT | ALS | NRI | HLT | | | | | |
| | | | | (SEQ ID | R | Q | T | Q | | | | | |
| | | | | NO: 661) | (SEQ | (SEQ | | | | | | | |
| | | | | | ID | ID | | | | | | | |
| | | | | | NO: | NO: | | | | | | | |
| | | | | | 881) | 886) | | | | | | | |
| R8-3i-ATP8 | ATP8 | Human | ATP8 | GTGGTA | TSG | QSG | RSD | — | — | — | LB | DddA^{N} | Canonical |
| | | | | GTT | SLS | SLT | ALS | | | | | | |
| | | | | | R | R | Q | | | | | | |
| | | | | | (SEQ | (SEQ | (SEQ | | | | | | |
| | | | | | ID | ID | ID | | | | | | |
| | | | | | NO: | NO: | NO: | | | | | | |
| | | | | | 889) | 881) | 886) | | | | | | |
| R8-3ii-ATP8 | ATP8 | Human | ATP8 | TAGGTG | QSG | RSD | RND | — | — | — | LB | DddA^{N} | Canonical |
| | | | | GTA | SLT | ALS | NRI | | | | | | |
| | | | | | R | Q | T | | | | | | |
| | | | | | (SEQ | (SEQ | | | | | | | |
| | | | | | ID | ID | | | | | | | |
| | | | | | NO: | NO: | | | | | | | |
| | | | | | 881) | 886) | | | | | | | |
| R8-3iii-ATP8 | ATP8 | Human | ATP8 | AGGTAG | RSD | RND | RSD | — | — | — | LB | DddA^{N} | Canonical |
| | | | | GTG | ALS | NRI | HLT | | | | | | |
| | | | | | Q | T | Q | | | | | | |
| | | | | | (SEQ | | | | | | | | |
| | | | | | ID | | | | | | | | |
| | | | | | NO: | | | | | | | | |
| | | | | | 886) | | | | | | | | |
| 4-4i-ATP8 | ATP8 | Human | ATP8 | CAAAGC | QAS | TSH | ERS | QSG | — | — | RT | DddA^{C} | Canonical |
| | | | | CCATAA | NLI | SLT | HLR | NLT | | | | | |
| | | | | (SEQ ID | S | E | E | E | | | | | |
| | | | | NO: 662) | (SEQ | (SEQ | (SEQ | (SEQ | | | | | |
| | | | | | ID | ID | ID | ID | | | | | |
| | | | | | NO: | NO: | NO: | NO: | | | | | |
| | | | | | 801) | 773) | 762) | 769) | | | | | |
| 4-4ii-ATP8 | ATP8 | Human | ATP8 | CACCAA | TSH | ERS | QSG | SKK | — | — | RT | DddA^{C} | Canonical |
| | | | | AGCCCA | SLT | HLR | NLT | ALT | | | | | |
| | | | | (SEQ ID | E | E | E | E | | | | | |
| | | | | NO: 663) | (SEQ | (SEQ | (SEQ | (SEQ | | | | | |
| | | | | | ID | ID | ID | ID | | | | | |
| | | | | | NO: | NO: | NO: | NO: | | | | | |
| | | | | | 773) | 762) | 769) | 770) | | | | | |
| 4-3i-ATP8 | ATP8 | Human | ATP8 | AGCCCA | QAS | TSH | ERS | — | — | — | RT | DddA^{C} | Canonical |
| | | | | TAA | NLI | SLT | HLR | | | | | | |
| | | | | | S | E | E | | | | | | |
| | | | | | (SEQ | (SEQ | (SEQ | | | | | | |
| | | | | | ID | ID | ID | | | | | | |
| | | | | | NO: | NO: | NO: | | | | | | |
| | | | | | 801) | 773) | 762) | | | | | | |
| 4-3ii-ATP8 | ATP8 | Human | ATP8 | CAAAGC | TSH | ERS | QSG | — | — | — | RT | DddA^{C} | Canonical |
| | | | | CCA | SLT | HLR | NLT | | | | | | |
| | | | | | E | E | E | | | | | | |
| | | | | | (SEQ | (SEQ | (SEQ | | | | | | |
| | | | | | ID | ID | ID | | | | | | |
| | | | | | NO: | NO: | NO: | | | | | | |
| | | | | | 773) | 762) | 769) | | | | | | |
| 4-3iii-ATP8 | ATP8 | Human | ATP8 | CACCAA | ERS | QSG | SKK | — | — | — | RT | DddA^{C} | Canonical |
| | | | | AGC | HLR | NLT | ALT | | | | | | |
| | | | | | E | E | E | | | | | | |
| | | | | | (SEQ | (SEQ | (SEQ | | | | | | |
| | | | | | ID | ID | ID | | | | | | |
| | | | | | NO: | NO: | NO: | | | | | | |
| | | | | | 762) | 769) | 770) | | | | | | |
| 10-4i-ATP8 | ATP8 | Human | ATP8 | CCATAA | QAS | QRA | QAS | TSH | — | — | RT | DddA^{C} | Canonical |
| | | | | AAATAA | NLI | NLR | NLI | SLT | | | | | |
| | | | | (SEQ ID | S | A | S | E | | | | | |
| | | | | NO: 664) | (SEQ | (SEQ | (SEQ | (SEQ | | | | | |
| | | | | | ID | ID | ID | ID | | | | | |
| | | | | | NO: | NO: | NO: | NO: | | | | | |
| | | | | | 801) | 753) | 801) | 773) | | | | | |
| 10-4ii-ATP8 | ATP8 | Human | ATP8 | AGCCCA | QRA | QAS | TSH | ERS | — | — | RT | DddA^{C} | Canonical |
| | | | | TAAAAA | NLR | NLI | SLT | HLR | | | | | |
| | | | | (SEQ ID | A | S | E | E | | | | | |
| | | | | NO: 665) | (SEQ | (SEQ | (SEQ | (SEQ | | | | | |
| | | | | | ID | ID | ID | ID | | | | | |
| | | | | | NO: | NO: | NO: | NO: | | | | | |
| | | | | | 753) | 801) | 773) | 762) | | | | | |
| 10-3i-ATP8 | ATP8 | Human | ATP8 | TAAAAA | QAS | QRA | QAS | — | — | — | RT | DddA^{C} | Canonical |
| | | | | TAA | NLI | NLR | NLI | | | | | | |
| | | | | | S | A | S | | | | | | |
| | | | | | (SEQ | (SEQ | (SEQ | | | | | | |
| | | | | | ID | ID | ID | | | | | | |
| | | | | | NO: | NO: | NO: | | | | | | |
| | | | | | 801) | 753) | 801) | | | | | | |
| 10-3ii-ATP8 | ATP8 | Human | ATP8 | CCATA | QRANLRA | QASNLIS | TSHSLT | — | — | — | RT | DddA^{C} | Canonical |
| | | | | AAAA | (SEQ | (SEQ | E (SEQ | | | | | | |
| | | | | | ID | ID | ID | | | | | | |
| | | | | | NO: | NO: | NO: | | | | | | |
| | | | | | 753) | 801) | 773) | | | | | | |
| 10-3iii-ATP8 | ATP8 | Human | ATP8 | AGCCC | QAS | TSH | ERS | — | — | — | RT | DddA^{C} | Canonical |
| | | | | ATAA | NLI | SLT | HLR | | | | | | |
| | | | | | S | E | E | | | | | | |
| | | | | | (SEQ | (SEQ | (SEQ | | | | | | |
| | | | | | ID | ID | ID | | | | | | |
| | | | | | NO: | NO: | NO: | | | | | | |
| | | | | | 801) | 773) | 762) | | | | | | |
| G24-R1b | COX1 | Human | COX1 | GAGG | TSH | TSG | RSD | — | — | — | LB | DddA^{N} | Canonical |
| | | | | CTCCA | SLT | ELV | NLV | | | | | | |
| | | | | | E | R | R | | | | | | |
| | | | | | (SEQ | (SEQ | (SEQ | | | | | | |
| | | | | | ID | ID | ID | | | | | | |
| | | | | | NO: | NO: | NO: | | | | | | |
| | | | | | 773) | 792) | 787) | | | | | | |
| G32-R1b | COX1 | Human | COX1 | GGAG | TSG | QSS | QRA | — | — | — | RB | DddA^{C} | N- |
| | | | | AAGAT | NLV | NLV | HLE | | | | | | terminal |
| | | | | | R | R | R | | | | | | |
| | | | | | (SEQ | (SEQ | (SEQ | | | | | | |
| | | | | | ID | ID | ID | | | | | | |
| | | | | | NO: | NO: | NO: | | | | | | |
| | | | | | 788) | 785) | 793) | | | | | | |
| G22-R13 | ND5 | Human | ND51 | GAGGT | RSD | QSS | RSD | — | — | — | LB | DddA^{N} | Canonical |
| | | | | ATGG | HLT | SLV | NLV | | | | | | |
| | | | | | T | R | R | | | | | | |
| | | | | | (SEQ | (SEQ | (SEQ | | | | | | |
| | | | | | ID | ID | ID | | | | | | |
| | | | | | NO: | NO: | NO: | | | | | | |
| | | | | | 811) | 797) | 787) | | | | | | |
| G24-R13 | ND5 | Human | ND51 | GCTG | TTG | DCR | TSG | — | — | — | RB | DddA^{C} | N- |
| | | | | CCAAT | NLT | DLA | ELV | | | | | | terminal |
| | | | | | V | R | R | | | | | | |
| | | | | | (SEQ | (SEQ | (SEQ | | | | | | |
| | | | | | ID | ID | ID | | | | | | |
| | | | | | NO: | NO: | NO: | | | | | | |
| | | | | | 756) | 790) | 792) | | | | | | |
| G32-R6a | ND6 | Human | ND62 | GCTGT | TSG | RSD | TSG | — | — | — | LB | DddA^{C} | Canonical |
| | | | | GGGT | HLV | ELV | ELV | | | | | | |
| | | | | | R | R | R | | | | | | |
| | | | | | (SEQ | (SEQ | (SEQ | | | | | | |
| | | | | | ID | ID | ID | | | | | | |
| | | | | | NO: | NO: | NO: | | | | | | |
| | | | | | 796) | 799) | 792) | | | | | | |
| G21-R6a | ND6 | Human | ND62 | ATGGA | QSS | RSD | RRD | — | — | — | RB | DddA^{N} | N- |
| | | | | GGTA | SLV | NLV | ELN | | | | | | terminal |
| | | | | | R | R | V | | | | | | |
| | | | | | (SEQ | (SEQ | (SEQ | | | | | | |
| | | | | | ID | ID | ID | | | | | | |
| | | | | | NO: | NO: | NO: | | | | | | |
| | | | | | 797) | 787) | 767) | | | | | | |
| G36-R6c | ND6 | Human | ND62 | GGGGT | RSD | TSG | RSD | — | — | — | LB | DddA^{N} | Canonical |
| | | | | TGAG | NLV | SLV | KLV | | | | | | |
| | | | | | R | R | R | | | | | | |
| | | | | | (SEQ | (SEQ | (SEQ | | | | | | |
| | | | | | ID | ID | ID | | | | | | |
| | | | | | NO: | NO: | NO: | | | | | | |
| | | | | | 787) | 800) | 795) | | | | | | |
| G212-R6c | ND6 | Human | ND62 | GAGGCA | RSD | QSG | RSD | — | — | — | RB | DddA^{C} | N- |
| | | | | TGG | HLT | DLR | NLV | | | | | | terminal |
| | | | | | T | R | R | | | | | | |
| | | | | | (SEQ | (SEQ | (SEQ | | | | | | |
| | | | | | ID | ID | ID | | | | | | |
| | | | | | NO: | NO: | NO: | | | | | | |
| | | | | | 811) | 789) | 787) | | | | | | |
| G33-V1 | ND1 | Human | V1 | GTGGCG | TSG | RSD | RSD | — | — | — | LB | DddA^{C} | Canonical |
| | | | | GGT | HLV | DLV | ELV | | | | | | |
| | | | | | R | R | R | | | | | | |
| | | | | | (SEQ | (SEQ | (SEQ | | | | | | |
| | | | | | ID | ID | ID | | | | | | |
| | | | | | NO: | NO: | NO: | | | | | | |
| | | | | | 796) | 791) | 799) | | | | | | |
| G35-V1 | ND1 | Human | V1 | GATGTA | RSD | QSS | TSG | — | — | — | RB | DddA^{N} | N- |
| | | | | GAG | NLV | SLV | NLV | | | | | | terminal |
| | | | | | R | R | R | | | | | | |
| | | | | | (SEQ | (SEQ | (SEQ | | | | | | |
| | | | | | ID | ID | ID | | | | | | |
| | | | | | NO: | NO: | NO: | | | | | | |
| | | | | | 787) | 797) | 788) | | | | | | |
| G22-V2 | COX1 | Human | V2 | GAGTAG | RSD | RED | RSD | — | — | — | LB | DddA^{N} | Canonical |
| | | | | GAG | NLV | NLH | NLV | | | | | | |
| | | | | | R | T | R | | | | | | |
| | | | | | (SEQ | (SEQ | (SEQ | | | | | | |
| | | | | | ID | ID | ID | | | | | | |
| | | | | | NO: | NO: | NO: | | | | | | |
| | | | | | 787) | 803) | 787) | | | | | | |
| G34-V2 | COX1 | Human | V2 | GTGGAG | DCR | RSD | RSD | — | — | — | RT | DddA^{C} | Canonical |
| | | | | GCC | DLA | NLV | ELV | | | | | | |
| | | | | | R | R | R | | | | | | |
| | | | | | (SEQ | (SEQ | (SEQ | | | | | | |
| | | | | | ID | ID | ID | | | | | | |
| | | | | | NO: | NO: | NO: | | | | | | |
| | | | | | 790) | 787) | 799) | | | | | | |
| G33-V5 | ND6 | Human | V5 | GTGGTG | TSG | RSD | RSD | — | — | — | LB | DddA^{N} | Canonical |
| | | | | GTT | SLV | ELV | ELV | | | | | | |
| | | | | | R | R | R | | | | | | |
| | | | | | (SEQ | (SEQ | (SEQ | | | | | | |
| | | | | | ID | ID | ID | | | | | | |
| | | | | | NO: | NO: | NO: | | | | | | |
| | | | | | 800) | 799) | 799) | | | | | | |
| G36-V5 | ND6 | Human | V5 | GTGGG | QSS | TSG | RSD | — | — | — | RB | DddA^{C} | N- |
| | | | | TGAA | NLV | HLV | ELV | | | | | | terminal |
| | | | | | R | R | R | | | | | | |
| | | | | | (SEQ | (SEQ | (SEQ | | | | | | |
| | | | | | ID | ID | ID | | | | | | |
| | | | | | NO: | NO: | NO: | | | | | | |
| | | | | | 785) | 796) | 799) | | | | | | |
| ND1-Left | ND1 | Human | JSK-ND1 | GATTGA | DSG | QSS | QSS | ISSN | — | — | LB | DddA^{N} | Canonical |
| | | | | GTAAAC | NLR | SLI | HLN | LQR | | | | | |
| | | | | (SEQ ID | V | R | V | | | | | | |
| | | | | NO: 666) | (SEQ | (SEQ | (SEQ | | | | | | |
| | | | | | ID | ID | ID | | | | | | |
| | | | | | NO: | NO: | NO: | | | | | | |
| | | | | | 754) | 884) | 883) | | | | | | |
| ND1-Right | ND1 | Human | JSK-ND1 | GATG | TKNS | SKK | VSS | ISSN | — | — | RB | DddA^{C} | N-terminal |
| | | | | CTCA | LTE | ALTE | TLIR | LQR | | | | | |
| | | | | CCCT | (SEQ | (SEQ | | | | | | | |
| | | | | (SEQ | ID | ID | | | | | | | |
| | | | | ID | NO: | NO: | | | | | | | |
| | | | | NO: | 776) | 770) | | | | | | | |
| | | | | 667) | | | | | | | | | |
| ND2-Left | ND2 | Human | JSK-ND2 | GATTAG | RED | RSD | RED | ISSN | — | — | LB | DddA^{N} | Canonical |
| | | | | GCGTAG | NLH | ELT | NLH | LQR | | | | | |
| | | | | (SEQ ID | T | R | T | | | | | | |
| | | | | NO: 668) | (SEQ | | (SEQ | | | | | | |
| | | | | | ID | | ID | | | | | | |
| | | | | | NO: | | NO: | | | | | | |
| | | | | | 803) | | 803) | | | | | | |
| ND2-Right | ND2 | Human | JSK-ND2 | GGAGTA | QSS | RSD | QSS | QVS | — | — | RB | DddA^{C} | N- |
| | | | | GTGTGA | HLN | ELT | SLI | HLT | | | | | terminal |
| | | | | (SEQ ID | V | R | R | R | | | | | |
| | | | | NO: 669) | (SEQ | | (SEQ | | | | | | |
| | | | | | ID | | ID | | | | | | |
| | | | | | NO: | | NO: | | | | | | |
| | | | | | 883) | | 884) | | | | | | |
| ND4L-Left | ND4L | Human | JSK-ND4L | GACTAG | DPG | RED | RED | DSS | — | — | LB | DddA^{N} | Canonical |
| | | | | TAGGGC | HLV | NLH | NLH | NLQ | | | | | |
| | | | | (SEQ ID | R | T | T | R | | | | | |
| | | | | NO: 670) | (SEQ | (SEQ | (SEQ | | | | | | |
| | | | | | ID | ID | ID | | | | | | |
| | | | | | NO: | NO: | NO: | | | | | | |
| | | | | | 794) | 803) | 803) | | | | | | |
| ND4L-Right | ND4L | Human | JSK-ND4L | AACACA | DPG | VK | SPA | DSG | — | — | RT | DddA^{C} | Canonical |
| | | | | TATGGC | HLV | DYL | DLT | NLR | | | | | |
| | | | | (SEQ ID | R | TK | R | V | | | | | |
| | | | | NO: 671) | (SEQ | (SEQ | (SEQ | (SEQ | | | | | |
| | | | | | ID | ID | ID | ID | | | | | |
| | | | | | NO: | NO: | NO: | NO:754) | | | | | |
| | | | | | 794) | 890) | 757) | | | | | | |
| ND4-Left | ND4 | Human | JSK-ND4 | GATATA | VK | QSS | QSN | ISS | — | — | LB | DddA^{N} | Canonical |
| | | | | AAATAT | DYL | NLI | TLK | NLQ | | | | | |
| | | | | (SEQ ID | TK | T | Q | R | | | | | |
| | | | | NO: 672) | (SEQ | | (SEQ | | | | | | |
| | | | | | ID | | ID | | | | | | |
| | | | | | NO: | | NO: | | | | | | |
| | | | | | 890) | | 882) | | | | | | |
| ND4-Right | ND4 | Human | JSK-ND4 | AACCAC | VK | THL | SKK | DSGN | — | — | RT | DddA^{C} | Canonical |
| | | | | ACTTAT | DYL | DLI | ALT | LRV | | | | | |
| | | | | (SEQ ID | TK | R | E | (SEQ | | | | | |
| | | | | NO: 673) | (SEQ | (SEQ | (SEQ | ID | | | | | |
| | | | | | ID | ID | ID | NO: | | | | | |
| | | | | | NO: | NO: | NO: | 754) | | | | | |
| | | | | | 890) | 760) | 770) | | | | | | |
| ND5-Left | ND5 | Human | JSK-ND5 | TGAAAC | VK | QSS | DSG | QSS | — | — | LB | DddA^{N} | Canonical |
| | | | | CGATAT | DYL | HLN | NLR | HLN | | | | | |
| | | | | (SEQ ID | TK | V | V | V | | | | | |
| | | | | NO: 674) | (SEQ | (SEQ | (SEQ | (SEQ | | | | | |
| | | | | | ID | ID | ID | ID | | | | | |
| | | | | | NO: | NO: | NO: | NO: | | | | | |
| | | | | | 890) | 883) | 754) | 883) | | | | | |
| ND5-Right | ND5 | Human | JSK- | AATCAT | RSD | VSS | VSS | VSS | — | — | RB | DddA^{C} | N- |
| | | | ND5 | GCTAAG | NLT | TLI | NLN | NLN | | | | | terminal |
| | | | | (SEQ | Q | R | V | V | | | | | |
| | | | | ID | (SEQ | | | | | | | | |
| | | | | NO: | ID | | | | | | | | |
| | | | | 675) | NO: | | | | | | | | |
| | | | | | 888) | | | | | | | | |
| ND52-Left | ND5 | Human | JSK-ND52 | GTGGGA | QST | QST | QVS | RSD | — | — | LB | DddA^{C} | Canonical |
| | | | | AGAAGA | HLTQ | HLTQ | HLTR | ELTR | | | | | |
| | | | | (SEQ | (SEQ | (SEQ | | | | | | | |
| | | | | ID | ID | ID | | | | | | | |
| | | | | NO: | NO: | NO: | | | | | | | |
| | | | | 676) | 885) | 885) | | | | | | | |
| ND52-Right | ND5 | Human | JSK-ND52 | TAGGAG | WPS | RED | KSS | RED | — | — | RB | DddA^{N} | N-terminal |
| | | | | TAGGGT | NLT | NLH | NLR | NLH | | | | | |
| | | | | (SEQ ID | R | T | R | T | | | | | |
| | | | | NO: 677) | (SEQ | (SEQ | (SEQ | (SEQ | | | | | |
| | | | | | ID NO: | ID NO: | ID NO: | ID NO: | | | | | |
| | | | | | 891) | 803) | 879) | 803) | | | | | |
| COX1-Left | COX1 | Human | JSK-COX1 | GTAAGA | QST | DSS | QST | QSS | — | — | LB | DddA^{N} | Canonical |
| | | | | GTCAGA | HLT | AKR | HLT | SLI | | | | | |
| | | | | (SEQ ID | Q | R | Q | R | | | | | |
| | | | | NO: 678) | (SEQ | | (SEQ | (SEQ | | | | | |
| | | | | | ID | | ID | ID | | | | | |
| | | | | | NO: 885) | | NO: 885) | NO: 884) | | | | | |
| COX1-Right | COX1 | Human | JSK- | CGAGCA | QSS | QVS | QSS | QSS | — | — | RB | DddA^{C} | N-terminal |
| | | | COX1 | GGAGTA | SLI | HLT | SLI | HLN | | | | | |
| | | | | (SEQ ID | R | R | R | V | | | | | |
| | | | | NO: 679) | (SEQ | | (SEQ | (SEQ | | | | | |
| | | | | | ID | | ID | ID | | | | | |
| | | | | | NO: | | NO: | NO: | | | | | |
| | | | | | 884) | | 884) | 883) | | | | | |
| COX2-Left | COX2 | Human | JSK- | TAGGAT | DPGHLVR | ISS | ISS | REDNLH | — | — | LB | DddA^{C} | Canonical |
| | | | COX2 | GATGGC | (SEQ | NLQR | NLQ | T | | | | | |
| | | | | (SEQ ID | ID | | R | (SEQ | | | | | |
| | | | | NO: 680) | NO: | | | ID | | | | | |
| | | | | | 794) | | | NO: | | | | | |
| | | | | | | | | 803) | | | | | |
| COX2-Right | COX2 | Human | JSK- | TAGGGA | KSS | RSD | QVS | RED | — | — | RB | DddA^{N} | N-terminal |
| | | | COX2 | TGGGAG | NLRR | HLKT | HLTR | NLHT | | | | | |
| | | | | (SEQ | (SEQ | | | (SEQ | | | | | |
| | | | | ID | ID | | | ID | | | | | |
| | | | | NO: | NO: | | | NO: | | | | | |
| | | | | 681) | 879) | | | 803) | | | | | |
| CYB-Left | CYTB | Human | JSK- | ATAGCC | QKSN | VK | DKS | QSNT | — | — | LB | DddA^{N} | Canonical |
| | | | CYB | TATGAA | LIR | DYL | CLN | LKQ | | | | | |
| | | | | (SEQ | (SEQ | TK | R | (SEQ | | | | | |
| | | | | ID | ID | (SEQ | | ID | | | | | |
| | | | | NO: | NO: | ID | | NO: | | | | | |
| | | | | 682) | 880) | NO: | | 882) | | | | | |
| | | | | | | 890) | | | | | | | |
| CYB-Right | CYTB | Human | JSK- | TGAGGC | QSN | QSS | DPG | QSSH | — | — | RT | DddA^{C} | Canonical |
| | | | CYB | CAAATA | TLK | NLI | HLV | LNV | | | | | |
| | | | | (SEQ | Q | V | R | (SEQ | | | | | |
| | | | | ID | (SEQ | | (SEQ | ID | | | | | |
| | | | | NO: | ID | | ID | NO: | | | | | |
| | | | | 683) | NO: | | NO: | 883) | | | | | |
| | | | | | 882) | | 794) | | | | | | |
| G21-MT-TK | MT-TK | Human | MT-TK | GTTA | TSG | QRAN | TSGS | — | — | — | LT | DddA^{N} | N-terminal |
| | | | | AAGAT | NLVR | LRA | LVR | | | | | or | |
| | | | | | (SEQ | (SEQ | (SEQ | | | | | DddA^{C} | |
| | | | | | ID | ID | ID | | | | | | |
| | | | | | NO: | NO: | NO: | | | | | | |
| | | | | | 788) | 753) | 800) | | | | | | |
| G11-MT-TK | MT-TK | Human | MT-TK | AAAGAT | QAS | TSG | QRA | — | — | — | LT | DddA^{N} | N- |
| | | | | TAA | NLI | NLV | NLR | | | | | or | terminal |
| | | | | | S | R | A | | | | | DddA^{C} | |
| | | | | | (SEQ | (SEQ | (SEQ | | | | | | |
| | | | | | ID | ID | ID | | | | | | |
| | | | | | NO: | NO: | NO: | | | | | | |
| | | | | | 801) | 788) | 753) | | | | | | |
| G12-MT-TK | MT-TK | Human | MT-TK | TAAGTT | QRA | TSG | QAS | — | — | — | LT | DddA^{N} | N- |
| | | | | AAA | NLR | SLV | NLI | | | | | or | terminal |
| | | | | | A | R | S | | | | | DddA^{C} | |
| | | | | | (SEQ | (SEQ | (SEQ | | | | | | |
| | | | | | ID | ID | ID | | | | | | |
| | | | | | NO: | NO: | NO: | | | | | | |
| | | | | | 753) | 800) | 801) | | | | | | |
| G31-MT-TK | MT-TK | Human | MT-TK | GGTGTT | TSG | TSG | TSG | — | — | — | RB | DddA^{N} | N- |
| | | | | GGT | HLV | SLV | HLV | | | | | or | terminal |
| | | | | | R | R | R | | | | | DddA^{C} | |
| | | | | | (SEQ | (SEQ | (SEQ | | | | | | |
| | | | | | ID | ID | ID | | | | | | |
| | | | | | NO: | NO: | NO: | | | | | | |
| | | | | | 796) | 800) | 796) | | | | | | |
| G22-MT-TK | MT-TK | Human | MT-TK | GTGTTG | TSG | RKD | RSD | — | — | — | RB | DddA^{N} | N- |
| | | | | GTT | SLV | ALR | ELV | | | | | or | terminal |
| | | | | | R | G | R | | | | | DddA^{C} | |
| | | | | | (SEQ | (SEQ | (SEQ | | | | | | |
| | | | | | ID | ID | ID | | | | | | |
| | | | | | NO: | NO: | NO: | | | | | | |
| | | | | | 800) | 815) | 799) | | | | | | |
| G23-MT-TK | MT-TK | Human | MT-TK | GAGGTG | RKD | RSD | RSD | — | — | — | RB | DddA^{N} | N- |
| | | | | TTG | ALR | ELV | NLV | | | | | or | terminal |
| | | | | | G | R | R | | | | | DddA^{C} | |
| | | | | | (SEQ | (SEQ | (SEQ | | | | | | |
| | | | | | ID | ID | ID | | | | | | |
| | | | | | NO: | NO: | NO: | | | | | | |
| | | | | | 815) | 799) | 787) | | | | | | |
| G24-MT-TK | MT-TK | Human | MT-TK | AGAGGT | TSG | TSG | QLA | — | — | — | RB | DddA^{T} | N- |
| | | | | GTT | SLV | HLV | HLR | | | | | or | terminal |
| | | | | | R | R | A | | | | | DddA^{C} | |
| | | | | | (SEQ | (SEQ | (SEQ | | | | | | |
| | | | | | ID | ID | ID | | | | | | |
| | | | | | NO: | NO: | NO: | | | | | | |
| | | | | | 800) | 796) | 761) | | | | | | |
| G25-MT-TK | MT-TK | Human | MT-TK | AAAGAG | RSD | RSD | QRA | — | — | — | RB | DddA^{N} | N- |
| | | | | GTG | ELV | NLV | NLR | | | | | or | terminal |
| | | | | | R | R | A | | | | | DddA^{C} | |
| | | | | | (SEQ | (SEQ | (SEQ | | | | | | |
| | | | | | ID | ID | ID | | | | | | |
| | | | | | NO: | NO: | NO: | | | | | | |
| | | | | | 799) | 787) | 753) | | | | | | |
| G21-MT- | MT- | Hu | MT- | TAAGTT | TSG | QRA | TSG | QAS | — | — | LT | DddA | N- |
| TK(4ZF) | TK | man | TK | AAAGAT | NLV | NLR | SLV | NLI | | | | | terminal |
| | | | | | R | A | R | S | | | | | |
| | | | | (SEQ | (SEQ | (SEQ | (SEQ | (SEQ | | | | | |
| | | | | ID | ID | ID | ID | ID | | | | | |
| | | | | NO: | NO: | NO: | NO: | NO: | | | | | |
| | | | | 684) | 788) | 753) | 800) | 801) | | | | | |
| G23-MT- | MT-TK | Human | MT- | AAAGAG | RKD | RSD | RSD | QRA | — | — | RB | DddA^{C} | N- |
| TK(4ZF) | | | TK | GTGTTG | ALR | ELV | NLV | NLR | | | | | terminal |
| | | | | (SEQ | G | R | R | A | | | | | |
| | | | | ID | (SEQ | (SEQ | (SEQ | (SEQ | | | | | |
| | | | | NO: | ID | ID | ID | ID | | | | | |
| | | | | 685) | NO: | NO: | NO: | NO: | | | | | |
| | | | | | 815) | 799) | 787) | 753) | | | | | |
| G23-MT- | MT-TK | Human | MT- | TGTAAA | RKD | RSD | RSD | QRA | WR | — | RB | DddA^{C} | N- |
| TK(5ZF) | | | TK | GAGGTG | ALR | ELV | NLV | NLR | DSL | | | | terminal |
| | | | | TTG | G | R | R | A | LA | | | | |
| | | | | (SEQ | (SEQ | (SEQ | (SEQ | (SEQ | (SEQ | | | | |
| | | | | ID NO: | ID | ID | ID | ID | ID | | | | |
| | | | | 686) | NO: | NO: | NO: | NO: | NO: | | | | |
| | | | | | 815) | 799) | 787) | 753) | 812) | | | | |
| G31-V1 | ND1 | Human | V1 | GTAGAT | RSD | TSG | QSS | — | — | — | LB | DddA^{N} | Canonical |
| | | | | GTG | ELV | NLV | SLV | | | | | | |
| | | | | | R | R | R | | | | | | |
| | | | | | (SEQ | (SEQ | (SEQ | | | | | | |
| | | | | | ID | ID | Q II | | | | | | |
| | | | | | NO: | NO: | NO: | | | | | | |
| | | | | | 799) | 788) | 797) | | | | | | |
| G32-V1 | ND1 | Human | V1 | GATGTG | RSD | RSD | TSG | — | — | — | LB | DddA^{N} | Canonical |
| | | | | GCG | DLV | ELV | NLV | | | | | | |
| | | | | | R | R | R | | | | | | |
| | | | | | (SEQ | (SEQ | (SEQ | | | | | | |
| | | | | | ID | ID | ID | | | | | | |
| | | | | | NO: | NO: | NO: | | | | | | |
| | | | | | 791) | 799) | 788) | | | | | | |
| G41-V1 | ND1 | Human | V1 | GTAGAT | RSD | RSD | TSG | QSS | — | — | LB | DddA^{N} | Canonical |
| | | | | GTGGCG | DLV | ELV | NLV | SLV | | | | | |
| | | | | (SEQ | R | R | R | R | | | | | |
| | | | | ID NO: | (SEQ | (SEQ | (SEQ | (SEQ | | | | | |
| | | | | 687) | ID | ID | ID | ID | | | | | |
| | | | | | NO: | NO: | NO: | NO: | | | | | |
| | | | | | 791) | 799) | 788) | 797) | | | | | |
| G42-V1 | ND1 | Human | V1 | GATGTG | TSG | RSD | RSD | TSG | — | — | LB | DddA^{N} | Canonical |
| | | | | GCGGGT | HLV | DLV | ELV | NLV | | | | | |
| | | | | (SEQ ID | R | R | R | R | | | | | |
| | | | | NO: 688) | (SEQ | (SEQ | (SEQ | (SEQ | | | | | |
| | | | | | ID | ID | ID | ID | | | | | |
| | | | | | NO: | NO: | NO: | NO: | | | | | |
| | | | | | 796) | 791) | 799) | 788) | | | | | |
| G51-V1 | ND1 | Human | V1 | GTAGAT | TSG | RSD | RSD | TSG | QSS | — | LB | DddA^{N} | Canonical |
| | | | | GTGGCG | HLV | DLV | ELV | NLV | SLV | | | | |
| | | | | GGT | R | R | R | R | R | | | | |
| | | | | (SEQ ID | (SEQ | (SEQ | (SEQ | (SEQ | (SEQ | | | | |
| | | | | NO: 689) | ID | ID | ID | ID | ID | | | | |
| | | | | | NO: | NO: | NO: | NO: | NO: | | | | |
| | | | | | 796) | 791) | 799) | 788) | 797) | | | | |
| G34-V1 | ND1 | Human | V1 | GTAGAG | TSG | RSD | QSS | — | — | — | RB | DddA^{C} | N- |
| | | | | GGT | HLV | NLV | SLV | | | | | | terminal |
| | | | | | R | R | R | | | | | | |
| | | | | | (SEQ | (SEQ | (SEQ | | | | | | |
| | | | | | ID | ID | ID | | | | | | |
| | | | | | NO: | NO: | NO: | | | | | | |
| | | | | | 796) | 787) | 797) | | | | | | |
| G35-V1 | ND1 | Human | V1 | GATG | RSD | QSS | TSG | — | — | — | RB | DddA^{C} | N-terminal |
| | | | | TAGAG | NLVR | SLVR | NLVR | | | | | | |
| | | | | | (SEQ | (SEQ | (SEQ | | | | | | |
| | | | | | ID | ID | ID | | | | | | |
| | | | | | NO: | NO: | NO: | | | | | | |
| | | | | | 787) | 797) | 788) | | | | | | |
| G36-V1 | ND1 | Human | V1 | GGTGAT | QSS | TSG | TSG | — | — | — | RB | DddA^{C} | N- |
| | | | | GTA | SLV | NLV | HLV | | | | | | terminal |
| | | | | | R | R | R | | | | | | |
| | | | | | (SEQ | (SEQ | (SEQ | | | | | | |
| | | | | | ID | ID | ID | | | | | | |
| | | | | | NO: | NO: | NO: | | | | | | |
| | | | | | 797) | 788) | 796) | | | | | | |
| G44-V1 | ND1 | Human | V1 | GATGTA | TSG | RSD | QSS | TSC | — | — | RB | DddA^{C} | N- |
| | | | | GAGGGT | HLV | NLV | SLV | NLV | | | | | terminal |
| | | | | (SEQ ID | R | R | R | R | | | | | |
| | | | | NO: 690) | (SEQ | (SEQ | (SEQ | (SEQ | | | | | |
| | | | | | ID | ID | ID | ID | | | | | |
| | | | | | NO: | NO: | NO: | NO: | | | | | |
| | | | | | 796) | 787) | 797) | 788) | | | | | |
| G45-V1 | ND1 | Human | V1 | GGTGAT | RSD | QSS | TSG | TSG | — | — | RB | DddA^{C} | N- |
| | | | | GTAGAG | NLV | SLV | NLV | HLV | | | | | terminal |
| | | | | (SEQ ID | R | R | R | R | | | | | |
| | | | | NO: 691) | (SEQ | (SEQ | (SEQ | (SEQ | | | | | |
| | | | | | ID | ID | ID | ID | | | | | |
| | | | | | NO: | NO: | NO: | NO: | | | | | |
| | | | | | 787) | 797) | 788) | 796) | | | | | |
| G46-V1 | ND1 | Human | V1 | GGCGGT | QSS | TSG | TSG | DPG | — | — | RB | DddA^{C} | N- |
| | | | | GATGTA | SLV | NLV | HLV | HLV | | | | | terminal |
| | | | | (SEQ ID | R | R | R | R | | | | | |
| | | | | NO: 692) | (SEQ | (SEQ | (SEQ | (SEQ | | | | | |
| | | | | | ID | ID | ID | ID | | | | | |
| | | | | | NO: | NO: | NO: | NO: | | | | | |
| | | | | | 797) | 788) | 796) | 794) | | | | | |
| G54-V1 | ND1 | Human | V1 | GGTGAT | TSG | RSD | QSS | TSG | TSG | — | RB | DddA^{C} | N- |
| | | | | GTAGAG | HLV | NLV | SLV | NLV | HLV | | | | terminal |
| | | | | GGT | R | R | R | R | R | | | | |
| | | | | (SEQ ID | (SEQ | (SEQ | (SEQ | (SEQ | (SEQ | | | | |
| | | | | NO: 693) | ID | ID | ID | ID | ID | | | | |
| | | | | | NO: | NO: | NO: | NO: | NO: | | | | |
| | | | | | 796) | 787) | 797) | 788) | 796) | | | | |
| G55-V1 | ND1 | Human | V1 | GGCGGT | RSD | QSS | TSG | TSG | DPG | — | RB | DddA^{C} | N- |
| | | | | GATGTA | NLV | SLV | NLV | HLV | HLV | | | | terminal |
| | | | | GAG | R | R | R | R | R | | | | |
| | | | | (SEQ ID | (SEQ | (SEQ | (SEQ | (SEQ | (SEQ | | | | |
| | | | | NO: 694) | ID | ID | ID | ID | ID | | | | |
| | | | | | NO: | NO: | NO: | NO: | NO: | | | | |
| | | | | | 787) | 797) | 788) | 796 | 794) | | | | |
| G31-V2 | COX1 | Human | V2 | GCAGGA | QSS | QRA | QSG | — | — | — | LB | DddA^{N} | Canonical |
| | | | | GTA | SLV | HLE | DLR | | | | | | |
| | | | | | R | R | R | | | | | | |
| | | | | | (SEQ | (SEQ | (SEQ | | | | | | |
| | | | | | ID | ID | ID | | | | | | |
| | | | | | NO: | NO: | NO: | | | | | | |
| | | | | | 797) | 793) | 789) | | | | | | |
| G32-V2 | COX1 | Human | V2 | GGAGTA | QRA | QSS | QRA | — | — | — | LB | DddA^{N} | Canc |
| | | | | GGA | HLE | SLV | HLE | | | | | | |
| | | | | | R | R | R | | | | | | |
| | | | | | (SEQ | (SEQ | (SEQ | | | | | | |
| | | | | | ID | ID | ID | | | | | | |
| | | | | | NO: | NO: | NO: | | | | | | |
| | | | | | 793) | 797) | 793) | | | | | | |
| G41-V2 | COX1 | Human | V2 | GCAGGA | QRA | QSS | QRA | QSG | — | — | LB | DddA^{N} | Canonical |
| | | | | GTAGGA | HLE | SLV | HLE | DLR | | | | | |
| | | | | (SEQ ID | R | R | R | R | | | | | |
| | | | | NO: 695) | (SEQ | (SEQ | (SEQ | (SEQ | | | | | |
| | | | | | ID | ID | ID | ID | | | | | |
| | | | | | NO: | NO: | NO: | NO: | | | | | |
| | | | | | 793) | 797) | 793) | 789) | | | | | |
| G42-V2 | COX1 | Human | V2 | GGAGTA | RSD | QRA | QSS | QRA | — | — | LB | DddA^{N} | Canonical |
| | | | | GGAGAG | NLV | HLE | SLV | HLE | | | | | |
| | | | | (SEQ ID | R | R | R | R | | | | | |
| | | | | NO: 696) | (SEQ | (SEQ | (SEQ | (SEQ | | | | | |
| | | | | | ID | ID | ID | ID | | | | | |
| | | | | | NO: | NO: | NO: | NO: | | | | | |
| | | | | | 787) | 793) | 797) | 793) | | | | | |
| G51-V2 | COX1 | Human | V2 | GCAGGA | RSD | QRA | QSS | QRA | QSG | — | LB | DddA^{N} | Canonical |
| | | | | GTAGGA | NLV | HLE | SLV | HLE | DLR | | | | |
| | | | | GAG | R | R | R | R | R | | | | |
| | | | | (SEQ ID | (SEQ | (SEQ | (SEQ | (SEQ | (SEQ | | | | |
| | | | | NO: 697) | ID | ID | ID | ID | ID | | | | |
| | | | | | NO: | NO: | NO: | NO: | NO: | | | | |
| | | | | | 787) | 793) | 797) | 793) | 789) | | | | |
| G34-V2 | COX1 | Human | V2 | GTGGAG | DCR | RSD | RSD | — | — | — | RT | DddA^{C} | Canonical |
| | | | | GCC | DLA | NLV | ELV | | | | | | |
| | | | | | R | R | R | | | | | | |
| | | | | | (SEQ | (SEQ | (SEQ | | | | | | |
| | | | | | ID | ID | ID | | | | | | |
| | | | | | NO: | NO: | NO: | | | | | | |
| | | | | | 790) | 787) | 799) | | | | | | |
| G35-V2 | COX1 | Human | V2 | GAGGCC | QRA | DCR | RSD | — | — | — | RT | DddA^{C} | Canonical |
| | | | | GGA | HLE | DLA | NLV | | | | | | |
| | | | | | R | R | R | | | | | | |
| | | | | | (SEQ | (SEQ | (SEQ | | | | | | |
| | | | | | ID | ID | ID | | | | | | |
| | | | | | NO: | NO: | NO: | | | | | | |
| | | | | | 793) | 790) | 787) | | | | | | |
| G36-V2 | COX1 | Human | V2 | GCCGGA | QSG | QRA | DCR | — | — | — | RT | DddA^{C} | Canonical |
| | | | | GCA | DLR | HLE | DLA | | | | | | |
| | | | | | R | R | R | | | | | | |
| | | | | | (SEQ | (SEQ | (SEQ | | | | | | |
| | | | | | ID | ID | ID | | | | | | |
| | | | | | NO: | NO: | NO: | | | | | | |
| | | | | | 789) | 793) | 790) | | | | | | |
| G44-V2 | COX1 | Human | V2 | GTGGAG | QRA | DCR | RSD | RSD | — | — | RT | DddA^{C} | Canonical |
| | | | | GCCGGA | HLE | DLA | NLV | ELV | | | | | |
| | | | | (SEQ ID | R | R | R | R | | | | | |
| | | | | NO: 698) | (SEQ | (SEQ | (SEQ | (SEQ | | | | | |
| | | | | | ID | ID | ID | ID | | | | | |
| | | | | | NO: | NO: | NO: | NO: | | | | | |
| | | | | | 793) | 790) | 787) | 799) | | | | | |
| G45-V2 | COX1 | Human | V2 | GAGGCC | QSG | QRA | DCR | RSD | — | — | RT | DddA^{C} | Canonical |
| | | | | GGAGCA | DLR | HLE | DLA | NLV | | | | | |
| | | | | (SEQ ID | R | R | R | R | | | | | |
| | | | | NO: 699) | (SEQ | (SEQ | (SEQ | (SEQ | | | | | |
| | | | | | ID | ID | ID | ID | | | | | |
| | | | | | NO: | NO: | NO: | NO: | | | | | |
| | | | | | 789) | 793) | 790) | 787) | | | | | |
| G46-V2 | COX1 | Human | V2 | GCCGGA | QRA | QSG | QRA | DCR | — | — | RT | DddA^{C} | Canonical |
| | | | | GCAGGA | HLER | DLRR | HLER | DLAR | | | | | |
| | | | | (SEQ ID | (SEQ | (SEQ | (SEQ | (SEQ | | | | | |
| | | | | NO: 700) | ID | ID | ID | ID | | | | | |
| | | | | | NO: | NO: | NO: | NO: | | | | | |
| | | | | | 793) | 789) | 793) | 790) | | | | | |
| G54-V2 | COX1 | Human | V2 | GTGGAG | QSG | QRA | DCR | RSD | RSD | — | RT | DddA^{C} | Canonical |
| | | | | GCCGGA | DLR | HLE | DLA | NLV | ELV | | | | |
| | | | | GCA | R | R | R | R | R | | | | |
| | | | | (SEQ ID | (SEQ | (SEQ | (SEQ | (SEQ | (SEQ | | | | |
| | | | | NO: 701) | ID | ID | ID | ID | ID | | | | |
| | | | | | NO: | NO: | NO: | NO: | NO: | | | | |
| | | | | | 789) | 793) | 790) | 787) | 799) | | | | |
| G55-V2 | COX1 | Human | V2 | GAGGCC | QRA | QSG | QRA | DCR | RSD | — | RT | DddA^{C} | Canonical |
| | | | | GGAGCA | HLE | DLR | HLE | DLA | NLV | | | | |
| | | | | GGA | R | R | R | R | R | | | | |
| | | | | (SEQ ID | (SEQ | (SEQ | (SEQ | (SEQ | (SEQ | | | | |
| | | | | NO: 702) | ID | ID | ID | ID | ID | | | | |
| | | | | | NO: | NO: | NO: | NO: | NO: | | | | |
| | | | | | 793) | 789) | 793) | 790) | 787) | | | | |
| G30-V3 | COX1 | Human | V3 | GGCGGG | DPG | RSD | DPG | — | — | — | LB | DddA^{N} | Canonical |
| | | | | GTC | ALV | KLV | HLV | | | | | | |
| | | | | | R | R | R | | | | | | |
| | | | | | (SEQ | (SEQ | (SEQ | | | | | | |
| | | | | | ID | ID | ID | | | | | | |
| | | | | | NO: | NO: | NO: | | | | | | |
| | | | | | 798) | 795) | 794) | | | | | | |
| G31-V3 | COX1 | Human | V3 | GGGGTC | QSS | DPG | RSD | — | — | — | LB | DddA^{N} | Canonical |
| | | | | GAA | NLV | ALV | KLV | | | | | | |
| | | | | | R | R | R | | | | | | |
| | | | | | (SEQ | (SEQ | (SEQ | | | | | | |
| | | | | | ID | ID | ID | | | | | | |
| | | | | | NO: | NO: | NO: | | | | | | |
| | | | | | 785) | 798) | 795) | | | | | | |
| G32-V3 | COX1 | Human | V3 | GTCGAA | QSS | QSS | DPG | — | — | — | LB | DddA^{N} | Canonical |
| | | | | GAA | NLV | NLV | ALV | | | | | | |
| | | | | | R | R | R | | | | | | |
| | | | | | (SEQ | (SEQ | (SEQ | | | | | | |
| | | | | | ID | ID | ID | | | | | | |
| | | | | | NO: | NO: | NO: | | | | | | |
| | | | | | 785) | 785) | 798) | | | | | | |
| G33-V3 | COX1 | Human | V3 | GAAGAA | TSG | QSS | QSS | — | — | — | LB | DddA^{N} | Canonical |
| | | | | GGT | HLV | NLV | NLV | | | | | | |
| | | | | | R | R | R | | | | | | |
| | | | | | (SEQ | (SEQ | (SEQ | | | | | | |
| | | | | | ID | ID | ID | | | | | | |
| | | | | | NO: | NO: | NO: | | | | | | |
| | | | | | 796) | 785) | 785) | | | | | | |
| G34-V3 | COX1 | Human | V3 | GAAGGT | TSG | TSG | QSS | — | — | — | LB | DddA^{N} | Canonical |
| | | | | GGT | HLV | HLV | NLV | | | | | | |
| | | | | | R | R | R | | | | | | |
| | | | | | (SEQ | (SEQ | (SEQ | | | | | | |
| | | | | | ID | ID | ID | | | | | | |
| | | | | | NO: | NO: | NO: | | | | | | |
| | | | | | 796) | 796) | 785) | | | | | | |
| G35-V3 | COX1 | Human | V3 | GGTGGT | TSG | TSG | TSG | — | — | — | LB | DddA^{N} | Canonical |
| | | | | GTT | SLV | HLV | HLV | | | | | | |
| | | | | | R | R | R | | | | | | |
| | | | | | (SEQ | (SEQ | (SEQ | | | | | | |
| | | | | | ID | ID | ID | | | | | | |
| | | | | | NO: | NO: | NO: | | | | | | |
| | | | | | 800) | 796) | 796 | | | | | | |
| G36-V3 | COX1 | Human | V3 | GGTGTT | RSD | TSG | TSG | — | — | — | LB | DddA^{N} | Canonical |
| | | | | GAG | NLV | SLV | HLV | | | | | | |
| | | | | | R | R | R | | | | | | |
| | | | | | (SEQ | (SEQ | (SEQ | | | | | | |
| | | | | | ID | ID | ID | | | | | | |
| | | | | | NO: | NO: | NO: | | | | | | |
| | | | | | 787) | 800) | 796) | | | | | | |
| G37-V3 | COX1 | Human | V3 | GTTGAG | TSG | RSD | TSG | — | — | — | LB | DddA^{N} | Canonical |
| | | | | GTT | SLV | NLV | SLV | | | | | | |
| | | | | | R | R | R | | | | | | |
| | | | | | (SEQ | (SEQ | (SEQ | | | | | | |
| | | | | | ID | ID | ID | | | | | | |
| | | | | | NO: | NO: | NO: | | | | | | |
| | | | | | 800) | 787) | 800) | | | | | | |
| G38-V3 | COX1 | Human | V3 | GAGGTT | RSD | TSG | RSD | — | — | — | LB | DddA^{N} | Canonical |
| | | | | GCG | DLV | SLV | NLV | | | | | | |
| | | | | | R | R | R | | | | | | |
| | | | | | (SEQ | (SEQ | (SEQ | | | | | | |
| | | | | | ID | ID | ID | | | | | | |
| | | | | | NO: | NO: | NO: | | | | | | |
| | | | | | 791) | 800) | 787) | | | | | | |
| G40-V3 | COX1 | Human | V3 | GGCGGG | QSS | DPG | RSD | DPG | — | — | LB | DddA^{N} | Canonical |
| | | | | GTCGAA | NLV | ALV | KLV | HLV | | | | | |
| | | | | (SEQ ID | R | R | R | R | | | | | |
| | | | | NO: 703) | (SEQ | (SEQ | (SEQ | (SEQ | | | | | |
| | | | | | ID | ID | ID | ID | | | | | |
| | | | | | NO: | NO: | NO: | NO: | | | | | |
| | | | | | 785) | 798) | 795) | 794) | | | | | |
| G41-V3 | COX1 | Human | V3 | GGGGTC | QSS | QSS | DPG | RSD | — | — | LB | DddA^{N} | Canonical |
| | | | | GAAGAA | NLV | NLV | ALV | KLV | | | | | |
| | | | | (SEQ ID | R | R | R | R | | | | | |
| | | | | NO: 704) | (SEQ | (SEQ | (SEQ | (SEQ | | | | | |
| | | | | | ID | ID | ID | ID | | | | | |
| | | | | | NO: | NO: | NO: | NO: | | | | | |
| | | | | | 785) | 785) | 798) | 795) | | | | | |
| G42-V3 | COX1 | Human | V3 | GTCGAA | TSG | QSS | QSS | DPG | — | — | LB | DddA^{N} | Canonical |
| | | | | GAAGGT | HLV | NLV | NLV | ALV | | | | | |
| | | | | (SEQ ID | R | R | R | R | | | | | |
| | | | | NO: 705) | (SEQ | (SEQ | (SEQ | (SEQ | | | | | |
| | | | | | ID | ID | 2 ID | ID | | | | | |
| | | | | | NO: | NO: | NO: | NO: | | | | | |
| | | | | | 796) | 785) | 785) | 798) | | | | | |
| G43-V3 | COX1 | Human | V3 | GAAGAA | TSG | TSG | QSS | QSS | — | — | LB | DddA^{N} | Canonical |
| | | | | GGTGGT | HLV | HLV | NLV | NLV | | | | | |
| | | | | (SEQ ID | R | R | R | R | | | | | |
| | | | | NO: 706) | (SEQ | (SEQ | (SEQ | (SEQ | | | | | |
| | | | | | ID | ID | ID | ID | | | | | |
| | | | | | NO: | NO: | NO: | NO: | | | | | |
| | | | | | 796) | 796) | 785) | 785) | | | | | |
| G44-V3 | COX1 | Human | V3 | GAAGGT | TSG | TSG | TSG | QSS | — | — | LB | DddA^{N} | Canonical |
| | | | | GGTGTT | SLV | HLV | HLV | NLV | | | | | |
| | | | | (SEQ ID | R | R | R | R | | | | | |
| | | | | NO: 707) | (SEQ | (SEQ | (SEQ | (SEQ | | | | | |
| | | | | | ID | ID | ID | ID | | | | | |
| | | | | | NO: | NO: | NO: | NO: | | | | | |
| | | | | | 800) | 796) | 796) | 785) | | | | | |
| G45-V3 | COX1 | Human | V3 | GGTGG | RSDN | TSGS | TSG | TSG | — | — | LB | DddA^{N} | Canonical |
| | | | | TGTT | LVR | LVR | HLVR | HLVR | | | | | |
| | | | | GAG | (SEQ | (SEQ | (SEQ | (SEQ | | | | | |
| | | | | (SEQ ID | ID | ID | ID | ID | | | | | |
| | | | | NO: | NO: | NO: | NO: | NO: | | | | | |
| | | | | 708) | 787) | 800) | 796) | 796) | | | | | |
| G46-V3 | COX1 | Human | V3 | GGTGTT | TSG | RSD | TSG | TSG | — | — | LB | DddA^{N} | Canonical |
| | | | | GAGGTT | SLV | NLV | SLV | HLV | | | | | |
| | | | | (SEQ ID | R | R | R | R | | | | | |
| | | | | NO: | (SEQ | (SEQ | (SEQ | (SEQ | | | | | |
| | | | | 709) | ID | ID | ID | ID | | | | | |
| | | | | | NO: | NO: | NO: | NO: | | | | | |
| | | | | | 800) | 787) | 800) | 796) | | | | | |
| G47-V3 | COX1 | Human | V3 | GTTGAG | RSD | TSG | RSD | TSG | — | — | LB | DddA^{N} | Canonical |
| | | | | GTTGCG | DLV | SLV | NLV | SLV | | | | | |
| | | | | (SEQ ID | R | R | R | R | | | | | |
| | | | | NO: 710) | (SEQ | (SEQ | (SEQ | (SEQ | | | | | |
| | | | | | ID | ID | ID | ID | | | | | |
| | | | | | NO: | NO: | NO: | NO: | | | | | |
| | | | | | 791) | 800) | 787) | 800) | | | | | |
| G48-V3 | COX1 | Human | V3 | GAGGTT | DPG | RSD | TSG | RSD | — | — | LB | DddA^{N} | Canonical |
| | | | | GCGGTC | ALV | DLV | SLV | NLV | | | | | |
| | | | | (SEQ ID | R | R | R | R | | | | | |
| | | | | NO: 711) | (SEQ | (SEQ | (SEQ | (SEQ | | | | | |
| | | | | | ID | ID | ID | ID | | | | | |
| | | | | | NO: | NO: | NO: | NO: | | | | | |
| | | | | | 798) | 791) | 800 | 787) | | | | | |
| G50-V3 | COX1 | Human | V3 | GGCGGG | QSS | QSS | DPG | RSD | DPG | — | LB | DddA^{N} | Canonical |
| | | | | GTCGAA | NLV | NLV | ALV | KLV | HLV | | | | |
| | | | | GAA | R | R | R | R | R | | | | |
| | | | | (SEQ ID | (SEQ | (SEQ | (SEQ | (SEQ | (SEQ | | | | |
| | | | | NO: 712) | ID | ID | ID | ID | ID | | | | |
| | | | | | NO: | NO: | NO: | NO: | NO: | | | | |
| | | | | | 785) | 785) | 798) | 795) | 794) | | | | |
| G51-V3 | COX1 | Human | V3 | GGGGTC | TSG | QSS | QSS | DPG | RSD | — | LB | DddA^{N} | Canonical |
| | | | | GAAGAA | HLV | NLV | NLV | ALV | KLV | | | | |
| | | | | GGT | R | R | R | R | R | | | | |
| | | | | (SEQ ID | (SEQ | (SEQ | (SEQ | (SEQ | (SEQ | | | | |
| | | | | NO: 713) | ID | ID | ID | ID | ID | | | | |
| | | | | | NO: | NO: | NO: | NO: | NO: | | | | |
| | | | | | 796) | 785) | 785) | 798) | 795) | | | | |
| G52-V3 | COX1 | Human | V3 | GTCGAA | TSG | TSG | QSS | QSS | DPG | — | LB | DddA^{N} | Canonical |
| | | | | GAAGGT | HLV | HLV | NLV | NLV | ALV | | | | |
| | | | | GGT | R | R | R | R | R | | | | |
| | | | | (SEQ ID | (SEQ | (SEQ | (SEQ | (SEQ | (SEQ | | | | |
| | | | | NO: 714) | ID | ID | ID | ID | ID | | | | |
| | | | | | NO: | NO: | NO: | NO: | NO: | | | | |
| | | | | | 796) | 796) | 785) | 785) | 798) | | | | |
| G53-V3 | COX1 | Human | V3 | GAAGAA | TSG | TSG | TSG | QSS | QSS | — | LB | DddA^{N} | Canonical |
| | | | | GGTGGT | SLV | HLV | HLV | NLV | NLV | | | | |
| | | | | GTT | R | R | R | R | R | | | | |
| | | | | (SEQ ID | (SEQ | (SEQ | (SEQ | (SEQ | (SEQ | | | | |
| | | | | NO: 715) | ID | ID | ID | ID | ID | | | | |
| | | | | | NO: | NO: | NO: | NO: | NO: | | | | |
| | | | | | 800) | 796) | 796) | 785) | 785) | | | | |
| G54-V3 | COX1 | Human | V3 | GAAGGT | RSD | TSG | TSG | TSG | QSS | — | LB | DddA^{N} | Canonical |
| | | | | GGTGTT | NLV | SLV | HLV | HLV | NLV | | | | |
| | | | | GAG | R(SEQ | R(SEQ | R(SEQ | R(SEQ | R(SEQ | | | | |
| | | | | (SEQ ID | ID | ID | ID | ID | ID | | | | |
| | | | | NO: 716) | NO: | NO: | NO: | NO: 796) | NO: | | | | |
| | | | | | 787) | 800) | 796) | | 785) | | | | |
| G55-V3 | COX1 | Human | V3 | GGTGGT | TSG | RSD | TSG | TSG | TSG | — | LB | DddA^{N} | Canonical |
| | | | | GTTGAG | SLV | NLV | SLV | HLV | HLV | | | | |
| | | | | GTT | R | R | R | R | R | | | | |
| | | | | (SEQ ID | (SEQ | (SEQ | (SEQ | (SEQ | (SEQ | | | | |
| | | | | NO: 717) | ID | ID | ID | ID | ID | | | | |
| | | | | | NO: | NO: | NO: | NO: | NO: | | | | |
| | | | | | 800) | 787) | 800) | 796) | 796) | | | | |
| G56-V3 | COX1 | Human | V3 | GGTGTT | RSD | TSG | RSD | TSG | TSG | — | LB | DddA^{N} | Canonical |
| | | | | GAGGTT | DLV | SLV | NLV | SLV | HLV | | | | |
| | | | | GCG | R | R | R | R | R | | | | |
| | | | | (SEQ ID | (SEQ | (SEQ | (SEQ | (SEQ | (SEQ | | | | |
| | | | | NO: 718) | ID | ID | ID | ID | ID | | | | |
| | | | | | NO: | NO: | NO: | NO: | NO: | | | | |
| | | | | | 791) | 800) | 787) | 800) | 796) | | | | |
| G57-V3 | COX1 | Human | V3 | GTTGAG | DPG | RSD | TSG | RSD | TSG | — | LB | DddA^{N} | Canonical |
| | | | | GTTGCG | ALV | DLV | SLV | NLV | SLV | | | | |
| | | | | GTC | R | R | R | R | R | | | | |
| | | | | (SEQ ID | (SEQ | (SEQ | (SEQ | (SEQ | (SEQ | | | | |
| | | | | NO: 719) | ID | ID | ID | ID | ID | | | | |
| | | | | | NO: | NO: | NO: | NO: | NO: | | | | |
| | | | | | 798) | 791) | 800) | 787) | 800) | | | | |
| G310-V3 | COX1 | Human | V3 | GCCGGA | QRA | QRA | DCR | — | — | — | RT | DddA^{C} | Canonical |
| | | | | GGA | HLE | HLE | DLA | | | | | | |
| | | | | | R | R | R | | | | | | |
| | | | | | (SEQ | (SEQ | (SEQ | | | | | | |
| | | | | | ID | ID | ID | | | | | | |
| | | | | | NO: | NO: | NO: | | | | | | |
| | | | | | 793) | 793) | 790) | | | | | | |
| G311-V3 | COX1 | Human | V3 | GGAGGA | QRA | QRA | QRA | — | — | — | RT | DddA^{C} | Canonical |
| | | | | GGA | HLE | HLE | HLE | | | | | | |
| | | | | | R | R | R | | | | | | |
| | | | | | (SEQ | (SEQ | (SEQ | | | | | | |
| | | | | | ID | ID | ID | | | | | | |
| | | | | | NO: | NO: | NO: | | | | | | |
| | | | | | 793) | 793) | 793) | | | | | | |
| G410-V3 | COX1 | Human | V3 | GCCGGA | QRA | QRA | QRA | DCR | — | — | RT | DddA^{C} | Canonical |
| | | | | GGAGGA | HLE | HLE | HLE | DLA | | | | | |
| | | | | (SEQ ID | R | R | R | R | | | | | |
| | | | | NO: 720) | (SEQ | (SEQ | (SEQ | (SEQ | | | | | |
| | | | | | ID | ID | ID | ID | | | | | |
| | | | | | NO: | NO: | NO: | NO: | | | | | |
| | | | | | 793) | 793) | 793) | 790) | | | | | |
| G411-V3 | COX1 | Human | V3 | GGAGGA | DPG | QRA | QRA | QRA | — | — | RT | DddA^{C} | Canonical |
| | | | | GGAGAC | NLV | HLE | HLE | HLE | | | | | |
| | | | | (SEQ ID | R | R | R | R | | | | | |
| | | | | NO: 721) | (SEQ | (SEQ | (SEQ | (SEQ | | | | | |
| | | | | | ID | ID | ID | ID | | | | | |
| | | | | | NO: | NO: | NO: | NO: | | | | | |
| | | | | | 786) | 793) | 793) | 793) | | | | | |
| G510-V3 | COX1 | Human | V3 | GCCGGA | DPG | QRA | QRA | QRA | DCR | — | RT | DddA^{C} | Canonical |
| | | | | GGAGGA | NLV | HLE | HLE | HLE | DLA | | | | |
| | | | | GAC | R | R | R | R | R | | | | |
| | | | | (SEQ ID | (SEQ | (SEQ | (SEQ | (SEQ | (SEQ | | | | |
| | | | | NO: 722) | ID | ID | ID | ID | ID | | | | |
| | | | | | NO: | NO: | NO: | NO: | NO: | | | | |
| | | | | | 786) | 793) | 793) | 793) | 790) | | | | |
| G31-V4 | COX2 | Human | V4 | GCCG | DPGALVR | QSSSLVR | DCRDLAR | — | — | — | LB | DddA^{N} | Canonical |
| | | | | TAGTC | (SEQ | (SEQ | (SEQ | | | | | | |
| | | | | | ID | ID | ID | | | | | | |
| | | | | | NO: | NO: | NO: | | | | | | |
| | | | | | 798) | 797) | 790) | | | | | | |
| G32-V4 | COX2 | Human | V4 | GTAGT | TSG | DPG | QSS | — | — | — | LB | DddA^{N} | Canonical |
| | | | | CGGT | HLV | ALV | SLV | | | | | | |
| | | | | | R | R | R | | | | | | |
| | | | | | (SEQ | (SEQ | (SEQ | | | | | | |
| | | | | | ID | ID | ID | | | | | | |
| | | | | | NO: | NO: | NO: | | | | | | |
| | | | | | 796) | 798) | 797) | | | | | | |
| G41-V4 | COX2 | Human | V4 | GCCGTA | TSG | DPG | QSS | DCR | — | — | LB | DddA^{N} | Canonical |
| | | | | GTCGGT | HLV | ALV | SLV | DLA | | | | | |
| | | | | (SEQ ID | R | R | R | R | | | | | |
| | | | | NO: 723) | (SEQ | (SEQ | (SEQ | (SEQ | | | | | |
| | | | | | ID | ID | ID | ID | | | | | |
| | | | | | NO: | NO: | NO: | NO: | | | | | |
| | | | | | 796) | 798) | 797) | 790) | | | | | |
| G42-V4 | COX2 | Human | V4 | GTAGTC | QSS | TSG | DPG | QSS | — | — | LB | DddA^{N} | Canc |
| | | | | GGTGTA | SLV | HLV | ALV | SLV | | | | | |
| | | | | (SEQ ID | R | R | R | R | | | | | |
| | | | | NO: 724) | (SEQ | (SEQ | (SEQ | (SEQ | | | | | |
| | | | | | ID | ID | ID | ID | | | | | |
| | | | | | NO: | NO: | NO: | NO: | | | | | |
| | | | | | 797) | 796) | 798) | 797) | | | | | |
| G51-V4 | COX2 | Human | V4 | GCCGTA | QSS | TSG | DPG | QSS | DCR | — | LB | DddA^{N} | Canonical |
| | | | | GTCGGT | SLV | HLV | ALV | SLV | DLA | | | | |
| | | | | GTA | R | R | R | R | R | | | | |
| | | | | (SEQ ID | (SEQ | (SEQ | (SEQ | (SEQ | (SEQ | | | | |
| | | | | NO: 725) | ID | ID | ID | ID | ID | | | | |
| | | | | | NO: | NO: | NO: | NO: | NO: | | | | |
| | | | | | 797) | 796) | 798) | 797) | 790) | | | | |
| G34-V4 | COX2 | Human | V4 | GTTGAA | TSG | QSS | TSG | — | — | — | RB | DddA^{C} | N- |
| | | | | GAT | NLV | NLV | SLV | | | | | | terminal |
| | | | | | R | R | R | | | | | | |
| | | | | | (SEQ | (SEQ | (SEQ | | | | | | |
| | | | | | ID | ID | ID | | | | | | |
| | | | | | NO: | NO: | NO: | | | | | | |
| | | | | | 788) | 785) | 800) | | | | | | |
| G35-V4 | COX2 | Human | V4 | GGAGTT | QSS | TSG | QRA | — | — | — | RB | DddA^{C} | N- |
| | | | | GAA | NLV | SLV | HLE | | | | | | terminal |
| | | | | | R | R | R | | | | | | |
| | | | | | (SEQ | (SEQ | (SEQ | | | | | | |
| | | | | | ID | ID | ID | | | | | | |
| | | | | | NO: | NO: | NO: | | | | | | |
| | | | | | 785) | 800) | 793) | | | | | | |
| G44-V4 | COX2 | Human | V4 | GGAGTT | TSG | QSS | TSG | QRA | — | — | RB | DddA^{C} | N- |
| | | | | GAAGAT | NLV | NLV | SLV | HLE | | | | | terminal |
| | | | | (SEQ ID | R | R | R | R | | | | | |
| | | | | NO: 726) | (SEQ | (SEQ | (SEQ | (SEQ | | | | | |
| | | | | | ID | ID | ID | ID | | | | | |
| | | | | | NO: | NO: | NO: | NO: | | | | | |
| | | | | | 788) | 785) | 800) | 793) | | | | | |
| G45-V4 | COX2 | Human | V4 | GTAGGA | QSS | TSG | QRA | QSS | — | — | RB | DddA^{C} | N- |
| | | | | GTTGAA | NLV | SLV | HLE | SLV | | | | | terminal |
| | | | | (SEQ | R | R | R | R | | | | | |
| | | | | ID | (SEQ | (SEQ | (SEQ | (SEQ | | | | | |
| | | | | NO: | ID | ID | ID | ID | | | | | |
| | | | | 727) | NO: | NO: | NO: | NO: | | | | | |
| | | | | | 785) | 800) | 793) | 797) | | | | | |
| G54-V4 | COX2 | Human | V4 | GTAGGA | TSG | QSS | TSG | QRA | QSS | — | RB | DddA^{C} | N- |
| | | | | GTTGAA | NLV | NLV | SLI | HLE | SLV | | | | terminal |
| | | | | GAT | R | R | R | R | R | | | | |
| | | | | (SEQ ID | (SEQ | (SEQ | (SEQ | (SEQ | (SEQ | | | | |
| | | | | NO: 728) | ID | ID | ID | ID | ID | | | | |
| | | | | | NO: | NO: | NO: | NO: | NO: 797) | | | | |
| | | | | | 788) | 785) | 800) | 793) | | | | | |
| G31-V5 | ND6 | Human | V5 | GATGGG | RSD | RSD | TSG | — | — | — | LB | DddA^{N} | Canonical |
| | | | | GTG | ELV | KLV | NLV | | | | | | |
| | | | | | R | R | R | | | | | | |
| | | | | | (SEQ | (SEQ | (SEQ | | | | | | |
| | | | | | ID | ID | ID | | | | | | |
| | | | | | NO: | NO: | NO: | | | | | | |
| | | | | | 799) | 795) | 788) | | | | | | |
| G32-V5 | ND6 | Human | V5 | GGGGTG | RSD | RSD | RSD | — | — | — | LB | DddA^{N} | Canonical |
| | | | | GTG | ELV | ELV | KLV | | | | | | |
| | | | | | R | R | R | | | | | | |
| | | | | | (SEQ | (SEQ | (SEQ | | | | | | |
| | | | | | ID | ID | ID | | | | | | |
| | | | | | NO: | NO: | NO: | | | | | | |
| | | | | | 799) | 799) | 795) | | | | | | |
| G33-V5 | ND6 | Human | V5 | GTGGTG | TSG | RSD | RSD | — | — | — | LB | DddA^{N} | Canonical |
| | | | | GTT | SLV | ELV | ELV | | | | | | |
| | | | | | R | R | R | | | | | | |
| | | | | | (SEQ | (SEQ | (SEQ | | | | | | |
| | | | | | ID | ID | ID | | | | | | |
| | | | | | NO: | NO: | NO: | | | | | | |
| | | | | | 800) | 799) | 799) | | | | | | |
| G34-V5 | ND6 | Human | V5 | GTGGTT | RSD | TSG | RSD | — | — | — | LB | DddA^{N} | Canonical |
| | | | | GTG | ELV | SLV | ELV | | | | | | |
| | | | | | R | R | R | | | | | | |
| | | | | | (SEQ | (SEQ | (SEQ | | | | | | |
| | | | | | ID | ID | ID | | | | | | |
| | | | | | NO: | NO: | NO: | | | | | | |
| | | | | | 799) | 800) | 799) | | | | | | |
| G41-V5 | ND6 | Human | V5 | GATGGG | RSD | RSD | RSD | TSG | — | — | LB | DddA^{N} | Canonical |
| | | | | GTGGTG | ELV | ELV | KLV | NLV | | | | | |
| | | | | (SEQ ID | R | R | R | R | | | | | |
| | | | | NO: 729) | (SEQ | (SEQ | (SEQ | (SEQ | | | | | |
| | | | | | ID | ID | ID | ID | | | | | |
| | | | | | NO: | NO: | NO: | NO: | | | | | |
| | | | | | 799) | 799) | 795) | 788) | | | | | |
| G42-V5 | ND6 | Human | V5 | GGGGTG | TSG | RSD | RSD | RSD | — | — | LB | DddA^{N} | Canonical |
| | | | | GTGGTT | SLV | ELV | ELV | KLV | | | | | |
| | | | | (SEQ ID | R | R | R | R | | | | | |
| | | | | NO: 730) | (SEQ | (SEQ | (SEQ | (SEQ | | | | | |
| | | | | | ID | ID | ID | ID | | | | | |
| | | | | | NO: | NO: | NO: | NO: | | | | | |
| | | | | | 800) | 799) | 799) | 795) | | | | | |
| G43-V5 | ND6 | Human | V5 | GTGGTG | RSD | TSG | RSD | RSD | — | — | LB | DddA^{N} | Canonical |
| | | | | GTTGTG | ELV | SLV | ELV | ELV | | | | | |
| | | | | (SEQ ID | R | R | R | R | | | | | |
| | | | | NO: 731) | (SEQ | (SEQ | (SEQ | (SEQ | | | | | |
| | | | | | ID | ID | ID | ID | | | | | |
| | | | | | NO: | NO: | NO: | NO: | | | | | |
| | | | | | 799) | 800) | 799) | 799) | | | | | |
| G44-V5 | ND6 | Human | V5 | GTGGTTG | QSSSLVR | RSDELVR | TSGSLVR | RSDELVR | — | — | LB | DddA^{N} | Canonical |
| | | | | TGGTA | (SEQ | (SEQ | (SEQ | (SEQ | | | | | |
| | | | | (SEQ ID | ID | ID | ID | ID | | | | | |
| | | | | NO: 732) | NO: | NO: | NO: | NO: | | | | | |
| | | | | | 797) | 799) | 800) | 799) | | | | | |
| G51-V5 | ND6 | Human | V5 | GATGGG | TSG | RSD | RSD | RSD | TSG | — | LB | DddA^{N} | Canonical |
| | | | | GTGGTG | SLV | ELV | ELV | KLV | NLV | | | | |
| | | | | GTT | R | R | R | R | R | | | | |
| | | | | (SEQ ID | (SEQ | (SEQ | (SEQ | (SEQ | (SEQ | | | | |
| | | | | NO: 733) | ID | ID | ID | ID | ID | | | | |
| | | | | | NO: | NO: | NO: | NO: | NO: | | | | |
| | | | | | 800) | 799) | 799) | 795) | 788) | | | | |
| G52-V5 | ND6 | Human | V5 | GGGGTG | RSD | TSG | RSD | RSD | RSD | — | LB | DddA^{N} | Canonical |
| | | | | GTGGTT | ELV | SLV | ELV | ELV | KLV | | | | |
| | | | | GTG | R | R | R | R | R | | | | |
| | | | | (SEQ ID | (SEQ | (SEQ | (SEQ | (SEQ | (SEQ | | | | |
| | | | | NO: 734) | ID | ID | ID | ID | ID | | | | |
| | | | | | NO: | NO: | NO: | NO: | NO: | | | | |
| | | | | | 799) | 800) | 799) | 799) | 795) | | | | |
| G53-V5 | ND6 | Human | V5 | GTGGTG | QSS | RSD | TSG | RSD | RSD | — | LB | DddA^{N} | Canonical |
| | | | | GTTGTG | SLV | ELV | SLV | ELV | ELV | | | | |
| | | | | GTA | R | R | R | R | R | | | | |
| | | | | (SEQ ID | (SEQ | (SEQ | (SEQ | (SEQ | (SEQ | | | | |
| | | | | NO: 735) | ID | ID | ID | ID | ID | | | | |
| | | | | | NO: | NO: | NO: | NO: | NO: | | | | |
| | | | | | 797) | 799) | 800) | 799) | 799) | | | | |
| G36-V5 | ND6 | Human | V5 | GTGGGT | QSS | TSG | RSD | — | — | — | RB | DddA^{C} | N- |
| | | | | GAA | NLV | HLV | ELV | | | | | | terminal |
| | | | | | R | R | R | | | | | | |
| | | | | | (SEQ | (SEQ | (SEQ | | | | | | |
| | | | | | ID | ID | ID | | | | | | |
| | | | | | NO: | NO: | NO: | | | | | | |
| | | | | | 785) | 796) | 799) | | | | | | |
| G37-V5 | ND6 | Human | V5 | GCTGTG | TSG | RSD | TSG | — | — | — | RB | DddA^{C} | N- |
| | | | | GGT | HLV | ELV | ELV | | | | | | terminal |
| | | | | | R | R | R | | | | | | |
| | | | | | (SEQ | (SEQ | (SEQ | | | | | | |
| | | | | | ID | ID | ID | | | | | | |
| | | | | | NO: | NO: | NO: | | | | | | |
| | | | | | 796) | 799) | 792) | | | | | | |
| G46-V5 | ND6 | Human | V5 | GCTGTG | QSS | TSG | RSD | TSG | — | — | RB | DddA^{C} | N- |
| | | | | GGTGAA | NLV | HLV | ELV | ELV | | | | | terminal |
| | | | | (SEQ ID | R | R | R | R | | | | | |
| | | | | NO: 736) | (SEQ | (SEQ | (SEQ | (SEQ | | | | | |
| | | | | | ID | ID | ID | ID | | | | | |
| | | | | | NO: | NO: | NO: | NO: | | | | | |
| | | | | | 785) | 796) | 799) | 792) | | | | | |
| G47-V5 | ND6 | Human | V5 | GGTGCT | TSG | RSD | TSG | TSG | — | — | RB | DddA^{C} | N- |
| | | | | GTGGGT | HLV | ELV | ELV | HLV | | | | | terminal |
| | | | | (SEQ ID | R | R | R | R | | | | | |
| | | | | NO: 737) | (SEQ | (SEQ | (SEQ | (SEQ | | | | | |
| | | | | | ID | ID | ID | ID | | | | | |
| | | | | | NO: | NO: | NO: | NO: | | | | | |
| | | | | | 796) | 799) | 792) | 796) | | | | | |
| G56-V5 | ND6 | Human | V5 | GGTGCT | QSS | TSG | RSD | TSG | TSG | — | RB | DddA^{C} | N- |
| | | | | GTGGGT | NLV | HLV | ELV | ELV | HLV | | | | terminal |
| | | | | GAA | R | R | R | R | R | | | | |
| | | | | (SEQ | (SEQ | (SEQ | (SEQ | (SEQ | (SEQ | | | | |
| | | | | ID | ID | ID | ID | ID | ID | | | | |
| | | | | NO: | NO: | NO: | NO: | NO: | NO: | | | | |
| | | | | 738) | 785) | 796) | 799) | 792) | 796) | | | | |
| LT30-Mt-tk | Mt-tk | Mouse | Mt-tk | AAGTTA | RSD | QK | RKD | — | — | — | LT | DddA^{N} | N- |
| | | | | GAG | NLV | WP | NLK | | | | | or | terminal |
| | | | | | R | RDS | N | | | | | DddA^{C} | |
| | | | | | (SEQ | (SEQ | (SEQ | | | | | | |
| | | | | | ID | ID | ID | | | | | | |
| | | | | | NO: | NO: | NO: | | | | | | |
| | | | | | 787) | 813) | 755) | | | | | | |
| LT31-Mt-tk | Mt-tk | Mouse | Mt-tk | AAAGTT | QLA | TSG | QRA | — | — | — | LT | DddA^{N} | N- |
| | | | | AGA | HLR | SLV | NLR | | | | | or | terminal |
| | | | | | A | R | A | | | | | DddA^{C} | |
| | | | | | (SEQ | (SEQ | (SEQ | | | | | | |
| | | | | | ID | ID | ID | | | | | | |
| | | | | | NO: | NO: | NO: | | | | | | |
| | | | | | 761) | 800) | 753) | | | | | | |
| LT32-Mt-tk | Mt-tk | Mouse | Mt-tk | TAAAGT | RED | HRT | QAS | — | — | — | LT | DddA^{N} | N- |
| | | | | TAG | NLH | TLT | NLI | | | | | or | terminal |
| | | | | | T | N | S | | | | | DddA^{C} | |
| | | | | | (SEQ | (SEQ | (SEQ | | | | | | |
| | | | | | ID | ID | ID | | | | | | |
| | | | | | NO: | NO: | NO: | | | | | | |
| | | | | | 803) | 764) | 801) | | | | | | |
| LT33-Mt-tk | Mt-tk | Mouse | Mt-tk | TTAAAG | QK | RKD | QK | — | — | — | LT | DddA^{N} | N- |
| | | | | TTA | WP | NLK | WP | | | | | or | terminal |
| | | | | | RDS | N | RDS | | | | | DddA^{C} | |
| | | | | | (SEQ | (SEQ | (SEQ | | | | | | |
| | | | | | ID | ID | ID | | | | | | |
| | | | | | NO: | NO: | NO: | | | | | | |
| | | | | | 813) | 755) | 813) | | | | | | |
| LT34-Mt-tk | Mt-tk | Mouse | Mt-tk | GTTAAA | TSG | QRA | TSG | — | — | — | LT | DddA^{N} | N- |
| | | | | GTT | SLV | NLR | SLV | | | | | or | terminal |
| | | | | | R | A | R | | | | | DddA^{C} | |
| | | | | | (SEQ | (SEQ | (SEQ | | | | | | |
| | | | | | ID | ID | ID | | | | | | |
| | | | | | NO: | NO: | NO: | | | | | | |
| | | | | | 800) | 753) | 800) | | | | | | |
| LT35-Mt-tk | Mt-tk | Mouse | Mt-tk | AGTTAA | HRT | QAS | HRT | — | — | — | LT | DddA^{N} | N- |
| | | | | AGT | TLT | NLI | TLT | | | | | or | terminal |
| | | | | | N | S | N | | | | | DddA^{C} | |
| | | | | | (SEQ | (SEQ | (SEQ | | | | | | |
| | | | | | ID | ID | ID | | | | | | |
| | | | | | NO: | NO: | NO: | | | | | | |
| | | | | | 764) | 801) | 764) | | | | | | |
| LT36-Mt-tk | Mt-tk | Mouse | Mt-tk | AAGTTA | RKD | QK | RKD | — | — | — | LT | DddA^{N} | N- |
| | | | | AAG | NLK | WP | NLK | | | | | or | terminal |
| | | | | | N | RDS | N | | | | | DddA^{C} | |
| | | | | | (SEQ | (SEQ | (SEQ | | | | | | |
| | | | | | ID | ID | ID | | | | | | |
| | | | | | NO: | NO: | NO: | | | | | | |
| | | | | | 755) | 813) | 755) | | | | | | |
| LT37-Mt-tk | Mt-tk | Mouse | Mt-tk | TAAGTT | QRA | TSG | QAS | — | — | — | LT | DddA^{N} | N- |
| | | | | AAA | NLR | SLV | NLI | | | | | or | terminal |
| | | | | | A | R | S | | | | | DddA^{C} | |
| | | | | | (SEQ | (SEQ | (SEQ | | | | | | |
| | | | | | ID | ID | ID | | | | | | |
| | | | | | NO: | NO: | NO: | | | | | | |
| | | | | | 753) | 800) | 801) | | | | | | |
| LT38-Mt-tk | Mt-tk | Mouse | Mt-tk | TTAAGTTAA | QAS | HRT | QK | — | — | — | LT | DddA^{N} | N- |
| | | | | | NLIS | TLTN | WPRDS | | | | | or | terminal |
| | | | | | (SEQ | (SEQ | (SEQ | | | | | DddA^{C} | |
| | | | | | ID | ID | ID | | | | | | |
| | | | | | NO: | NO: | NO: | | | | | | |
| | | | | | 801) | 764) | 813) | | | | | | |
| LT311-Mt-tk | Mt-tk | Mouse | Mt-tk | CTTTTAAGT | HRT | QK | TTG | — | — | — | LT | DddA^{N} | N- |
| | | | | | TLT | WP | ALT | | | | | or | terminal |
| | | | | | N | RDS | E | | | | | DddA^{C} | |
| | | | | | (SEQ | (SEQ | (SEQ | | | | | | |
| | | | | | ID | ID | ID | | | | | | |
| | | | | | NO: | NO: | NO: | | | | | | |
| | | | | | 764) | 813) | 784) | | | | | | |
| LB30-Mt-tk | Mt-tk | Mouse | Mt-tk | CTCTAACTT | TTG | QAS | QRH | — | — | — | LB | DddA^{N} | Canonical |
| | | | | | ALT | NLI | HLV | | | | | or | |
| | | | | | E | S | E | | | | | DddA^{C} | |
| | | | | | (SEQ | (SEQ | (SEQ | | | | | | |
| | | | | | ID | ID | ID | | | | | | |
| | | | | | NO: 784) | NO: | NO: 782) | | | | | | |
| | | | | | | 801) | | | | | | | |
| LB32-Mt-tk | Mt-tk | Mouse | Mt-tk | CTAACTTTA | QK | THL | QNS | — | — | — | LB | DddA^{N} | Canonical |
| | | | | | WP | DLI | TLT | | | | | or | |
| | | | | | RDS | R | E | | | | | DddA^{C} | |
| | | | | | (SEQ | (SEQ | (SEQ | | | | | | |
| | | | | | ID | ID | ID | | | | | | |
| | | | | | NO: 813) | NO: | NO: | | | | | | |
| | | | | | | 760) | 781) | | | | | | |
| LB33-Mt-tk | Mt-tk | Mouse | Mt-tk | TAACTTTAA | QAS | TTG | QAS | — | — | — | LB | DddA^{N} | Canonical |
| | | | | | NLI | ALT | NLI | | | | | or | |
| | | | | | S | E | S | | | | | DddA^{C} | |
| | | | | | (SEQ | (SEQ | (SEQ | | | | | | |
| | | | | | ID | ID | ID | | | | | | |
| | | | | | NO: 801) | NO: | NO: 801) | | | | | | |
| | | | | | | 784) | | | | | | | |
| LB35-Mt-tk | Mt-tk | Mouse | Mt-tk | ACTTTAACT | THL | QK | THL | — | — | — | LB | DddA^{N} | Canonical |
| | | | | | DLI | WP | DLI | | | | | or | |
| | | | | | R | RDS | R | | | | | DddA^{C} | |
| | | | | | (SEQ | (SEQ | (SEQ | | | | | | |
| | | | | | ID | ID | ID | | | | | | |
| | | | | | NO: 760) | NO: 813) | NO: 760) | | | | | | |
| LB36-Mt-tk | Mt-tk | Mouse | Mt-tk | CTTTAACTT | TTG | QAS | TTG | — | — | — | LB | DddA^{N} | Canonical |
| | | | | | ALT | NLI | ALT | | | | | or | |
| | | | | | E | S | E | | | | | DddA^{C} | |
| | | | | | (SEQ | (SEQ | (SEQ | | | | | | |
| | | | | | ID | ID | ID | | | | | | |
| | | | | | NO: 784) | NO: 801) | NO: 784) | | | | | | |
| LB38-Mt-tk | Mt-tk | Mouse | Mt-tk | TTAACTTAA | QAS | THL | QK | — | — | — | LB | DddA^{N} | Canonical |
| | | | | | NLI | DLI | WP | | | | | or | |
| | | | | | S | R | RDS | | | | | DddA^{C} | |
| | | | | | (SEQ | (SEQ | (SEQ | | | | | | |
| | | | | | ID | ID | ID | | | | | | |
| | | | | | NO: 801) | NO: | NO: 813) | | | | | | |
| | | | | | | 760) | | | | | | | |
| LB39-Mt-tk | Mt-tk | Mouse | Mt-tk | TAACTTAAA | QRA | TTG | QAS | — | — | — | LB | DddA^{N} | Canonical |
| | | | | | NLR | ALT | NLI | | | | | or | |
| | | | | | A | E | S | | | | | DddA^{C} | |
| | | | | | (SEQ | (SEQ | (SEQ | | | | | | |
| | | | | | ID | ID | ID | | | | | | |
| | | | | | NO: | NO: | NO: | | | | | | |
| | | | | | 753) | 784) | 801) | | | | | | |
| LB310-Mt-tk | Mt-tk | Mouse | Mt-tk | AACTTAAAA | QRA | QK | DSG | — | — | — | LB | DddA^{N} | Canonical |
| | | | | | NLR | WP | NLR | | | | | or | |
| | | | | | A | RDS | V | | | | | DddA^{C} | |
| | | | | | (SEQ | (SEQ | (SEQ | | | | | | |
| | | | | | ID | ID | ID | | | | | | |
| | | | | | NO: | NO: | NO: | | | | | | |
| | | | | | 753) | 813) | 754) | | | | | | |
| LB311-Mt-tk | Mt-tk | Mouse | Mt-tk | ACTTAAAAG | RKD | QAS | THL | — | — | — | LB | DddA^{T} | Canonical |
| | | | | | NLK | NLI | DLI | | | | | or | |
| | | | | | N | S | R | | | | | DddA^{C} | |
| | | | | | (SEQ | (SEQ | (SEQ | | | | | | |
| | | | | | ID | ID | ID | | | | | | |
| | | | | | NO: | NO: | NO: | | | | | | |
| | | | | | 755) | 801) | 760) | | | | | | |
| LB312-Mt-tk | Mt-tk | Mouse | Mt-tk | CTTAAAAGG | RSD | QRA | TTG | — | — | — | LB | DddA^{N} | Canonical |
| | | | | | HLT | NLR | ALT | | | | | or | |
| | | | | | N | A | E | | | | | DddA^{C} | |
| | | | | | (SEQ | (SEQ | (SEQ | | | | | | |
| | | | | | ID | ID | ID | | | | | | |
| | | | | | NO: | NO: | NO: | | | | | | |
| | | | | | 763) | 753) | 784) | | | | | | |
| RT30-Mt-tk | Mt-tk | Mouse | Mt-tk | ACCTTAAAA | QRA | QK | DK | — | — | — | RT | DddA^{N} | Canonical |
| | | | | | NLR | WP | KDL | | | | | or | |
| | | | | | A | RDS | TR | | | | | DddA^{C} | |
| | | | | | (SEQ | (SEQ | (SEQ | | | | | | |
| | | | | | ID | ID | ID | | | | | | |
| | | | | | NO: | NO: | NO: | | | | | | |
| | | | | | 753) | 813) | 758) | | | | | | |
| RT31-Mt-tk | Mt-tk | Mouse | Mt-tk | CCTTAAAAT | TTG | QAS | TKN | — | — | — | RT | DddA^{N} | Canonical |
| | | | | | NLT | NLI | SLT | | | | | or | |
| | | | | | V | S | E | | | | | DddA^{C} | |
| | | | | | (SEQ | (SEQ | (SEQ | | | | | | |
| | | | | | ID | ID | ID | | | | | | |
| | | | | | NO: | NO: | NO: | | | | | | |
| | | | | | 756) | 801) | 776) | | | | | | |
| RT32-Mt-tk | Mt-tk | Mouse | Mt-tk | CTTAAAATC | RRS | QRA | TTG | — | — | — | RT | DddA^{N} | Canonical |
| | | | | | ACR | NLR | ALT | | | | | or | |
| | | | | | R | A | E | | | | | DddA^{C} | |
| | | | | | (SEQ | (SEQ | (SEQ | | | | | | |
| | | | | | ID | ID | ID | | | | | | |
| | | | | | NO: | NO: | NO: | | | | | | |
| | | | | | 766) | 753) | 784) | | | | | | |
| RT33-Mt-tk | Mt-tk | Mouse | Mt-tk | TTAAAATCT | RLR | QRA | QK | — | — | — | RT | DddA^{N} | Canonical |
| | | | | | DIQ | NLR | WP | | | | | or | |
| | | | | | F | A | RDS | | | | | DddA^{C} | |
| | | | | | (SEQ | (SEQ | (SEQ | | | | | | |
| | | | | | ID | ID | ID | | | | | | |
| | | | | | NO: | NO: | NO: | | | | | | |
| | | | | | 808) | 753) | 813) | | | | | | |
| RT34-Mt-tk | Mt-tk | Mouse | Mt-tk | TAAAATCTC | QRH | TTG | QAS | — | — | — | RT | DddA^{N} | Canonical |
| | | | | | HLV | NLT | NLI | | | | | or | |
| | | | | | E | V | S | | | | | DddA^{C} | |
| | | | | | (SEQ | (SEQ | (SEQ | | | | | | |
| | | | | | ID | ID | ID | | | | | | |
| | | | | | NO: | NO: | NO: | | | | | | |
| | | | | | 782) | 756) | 801 | | | | | | |
| RT35-Mt-tk | Mt-tk | Mouse | Mt-tk | AAAATCTCC | RSD | RRS | QRA | — | — | — | RT | DddA^{N} | Canonical |
| | | | | | ERKR | ACRR | NLRA | | | | | or | |
| | | | | | (SEQ | (SEQ | (SEQ | | | | | | |
| | | | | | ID | ID | ID | | | | | | |
| | | | | | NO: | NO: | NO: | | | | | | |
| | | | | | 806) | 766) | 753) | | | | | | |
| RT36-Mt-tk | Mt-tk | Mouse | Mt-tk | AAATCTCCA | TSH | RLR | QRA | — | — | — | RT | DddA^{N} | Canonical |
| | | | | | SLT | DIQ | NLR | | | | | or | |
| | | | | | E | F | A | | | | | DddA^{C} | |
| | | | | | (SEQ | (SEQ | (SEQ | | | | | | |
| | | | | | ID | ID | ID | | | | | | |
| | | | | | NO: | NO: | NO: | | | | | | |
| | | | | | 773) | 808) | 753) | | | | | | |
| RT37-Mt-tk | Mt-tk | Mouse | Mt-tk | AATCTCCAT | TSG | QRH | TTG | — | — | — | RT | DddA^{N} | Canonical |
| | | | | | NLT | HLV | NLT | | | | | or | |
| | | | | | E | E | V | | | | | DddA^{C} | |
| | | | | | (SEQ | (SEQ | (SEQ | | | | | | |
| | | | | | ID | ID | ID | | | | | | |
| | | | | | NO: | NO: | NO: | | | | | | |
| | | | | | 772) | 782) | 756) | | | | | | |
| RT38-Mt-tk | Mt-tk | Mouse | Mt-tk | ATCTCCATA | QKS | RSD | RRS | — | — | — | RT | DddA^{N} | Canonical |
| | | | | | SLI | ERK | ACR | | | | | or | |
| | | | | | A | R | R | | | | | DddA^{C} | |
| | | | | | (SEQ | (SEQ | (SEQ | | | | | | |
| | | | | | ID | ID | ID | | | | | | |
| | | | | | NO: | NO: | NO: | | | | | | |
| | | | | | 765) | 806) | 766) | | | | | | |
| RT39-Mt-tk | Mt-tk | Mouse | Mt-tk | TCTCCATAG | RED | TSH | RLR | — | — | — | RT | DddA^{N} | Canonical |
| | | | | | NLH | SLT | DIQ | | | | | or | |
| | | | | | T | E | F | | | | | DddA^{C} | |
| | | | | | (SEQ | (SEQ | (SEQ | | | | | | |
| | | | | | ID | ID | ID | | | | | | |
| | | | | | NO: | NO: | NO: | | | | | | |
| | | | | | 803) | 773) | 808 | | | | | | |
| RT310-Mt-tk | Mt-tk | Mouse | Mt-tk | CTCCATAGT | HRT | TSG | QRH | — | — | — | RT | DddA^{N} | Canonical |
| | | | | | TLT | NLT | HLV | | | | | or | |
| | | | | | N | E | E | | | | | DddA^{C} | |
| | | | | | (SEQ | (SEQ | (SEQ | | | | | | |
| | | | | | ID | ID | ID | | | | | | |
| | | | | | NO: | NO: | NO: | | | | | | |
| | | | | | 764) | 772) | 782) | | | | | | |
| RT311-Mt-tk | Mt-tk | Mouse | Mt-tk | TCCATAGTG | RSD | QKS | RSD | — | — | — | RT | DddA^{N} | Canonical |
| | | | | | ELV | SLI | ERK | | | | | or | |
| | | | | | R | A | R | | | | | DddA^{C} | |
| | | | | | (SEQ | (SEQ | (SEQ | | | | | | |
| | | | | | ID | ID | ID | | | | | | |
| | | | | | NO: | NO: | NO: | | | | | | |
| | | | | | 799) | 765) | 806) | | | | | | |
| RB31-Mt-tk | Mt-tk | Mouse | Mt-tk | ATTTTAAGG | RSD | QK | HK | — | — | — | RB | DddA^{N} | N- |
| | | | | | HLT | WP | | | | | | or | terminal |
| | | | | | N | RDS | QN | | | | | DddA^{C} | |
| | | | | | (SEQ | (SEQ | (SEQ | | | | | | |
| | | | | | ID | ID | ID | | | | | | |
| | | | | | NO: | NO: | NO: | | | | | | |
| | | | | | 763) | 813) | 768) | | | | | | |
| RB34-Mt-tk | Mt-tk | Mouse | Mt-tk | GAGATTTTA | QK | HK | RSD | — | — | — | RB | DddA^{N} | N- |
| | | | | | WP | | NLV | | | | | or | terminal |
| | | | | | RDS | QN | R | | | | | DddA^{C} | |
| | | | | | (SEQ | (SEQ | (SEQ | | | | | | |
| | | | | | ID | ID | ID | | | | | | |
| | | | | | NO: | NO: | NO: | | | | | | |
| | | | | | 813) | 768) | 787) | | | | | | |
| RB37-Mt-tk | Mt-tk | Mouse | Mt-tk | ATGGAG | HK | RSD | RRD | — | — | — | RB | DddA^{N} | N- |
| | | | | ATT | | NLV | ELN | | | | | or | terminal |
| | | | | | QN | R | V | | | | | DddA^{C} | |
| | | | | | (SEQ | (SEQ | (SEQ | | | | | DddA^{N} | |
| | | | | | ID | ID | ID | | | | | | |
| | | | | | NO: | NO: | NO: | | | | | | |
| | | | | | 768) | 787) | 767) | | | | | | |
| RB38-Mt-tk | Mt-tk | Mouse | Mt-tk | TATGGA | TSG | QRA | ARG | — | — | — | RB | DddA^{N} | N- |
| | | | | GAT | NLVR | HLER | NLRT | | | | | or | terminal |
| | | | | | (SEQ | (SEQ | (SEQ | | | | | DddA^{C} | |
| | | | | | ID | ID | ID | | | | | | |
| | | | | | NO: | NO: | NO: | | | | | | |
| | | | | | 788) | 793) | 804) | | | | | | |
| RB39-Mt-tk | Mt-tk | Mouse | Mt-tk | CTATGG | QLA | RSD | QNS | — | — | — | RB | DddA^{N} | N- |
| | | | | AGA | HLR | HLT | TLT | | | | | or | terminal |
| | | | | | A | T | E | | | | | DddA^{C} | |
| | | | | | (SEQ | (SEQ | (SEQ | | | | | | |
| | | | | | ID | ID | ID | | | | | | |
| | | | | | NO: | NO: | NO: | | | | | | |
| | | | | | 761) | 811) | 781) | | | | | | |
| RB310-Mt-tk | Mt-tk | Mouse | Mt-tk | ACTATG | RSD | RRD | THL | — | — | — | RB | DddA^{N} | N- |
| | | | | GAG | NLV | ELN | DLI | | | | | or | terminal |
| | | | | | R | V | R | | | | | DddA^{C} | |
| | | | | | (SEQ | (SEQ | (SEQ | | | | | | |
| | | | | | ID | ID | ID | | | | | | |
| | | | | | NO: | NO: | NO: | | | | | | |
| | | | | | 787) | 767) | 760) | | | | | | |
| RB311-Mt-tk | Mt-tk | Mouse | Mt-tk | CACTAT | QRA | ARG | SKK | — | — | — | RB | DddA^{N} | N- |
| | | | | GGA | HLE | NLR | ALT | | | | | or | terminal |
| | | | | | R | T | E | | | | | DddA^{C} | |
| | | | | | (SEQ | (SEQ | (SEQ | | | | | | |
| | | | | | ID | ID | ID | | | | | | |
| | | | | | NO: | NO: | NO: | | | | | | |
| | | | | | 793) | 804) | 770) | | | | | | |
| LT41-Mt-tk | Mt-tk | Mouse | Mt-tk | GTTAAA | QLA | TSG | QRA | TSG | — | — | LT | DddA^{C} | N- |
| | | | | GTTAGA | HLR | SLV | NLR | SLV | | | | | terminal |
| | | | | (SEQ ID | A | R | A | R | | | | | |
| | | | | NO: 739) | (SEQ | (SEQ | (SEQ | (SEQ | | | | | |
| | | | | | ID | ID | ID | ID | | | | | |
| | | | | | NO: | NO: | NO: | NO: | | | | | |
| | | | | | 761) | 800) | 753) | 800) | | | | | |
| LT51-Mt-tk | Mt-tk | Mouse | Mt-tk | TAAGTTA | QLA | TSG | QRA | TSG | QAS | — | LT | DddA^{C} | N-terminal |
| | | | | AAGTTAG | HLR | SLV | NLR | SLV | NLI | | | | |
| | | | | A | A | R | A | R | S | | | | |
| | | | | (SEQ ID | (SEQ | (SEQ | (SEQ | (SEQ | (SEQ | | | | |
| | | | | NO: 740) | ID | ID | ID | ID | ID | | | | |
| | | | | | NO: | NO: | NO: | NO: | NO: | | | | |
| | | | | | 761) | 800) | 753) | 800) | 801) | | | | |
| RB47-Mt-tk | Mt-tk | Mouse | Mt-tk | ACTATG | HK | RSD | RRD | THL | — | — | RB | DddA^{N} | N- |
| | | | | GAGATT | | NLV | ELN | DLI | | | | | terminal |
| | | | | (SEQ ID | QN | R | V | R | | | | | |
| | | | | NO: 741) | (SEQ | (SEQ | (SEQ | (SEQ | | | | | |
| | | | | | ID | ID | ID | ID | | | | | |
| | | | | | NO: | NO: | NO: | NO: | | | | | |
| | | | | | 768) | 787) | 767) | 760) | | | | | |
| RB48-Mt-tk | Mt-tk | Mouse | Mt-tk | CACTAT | TSGNLVR | QRAHLER | ARGNLRT | SKKALTE | — | — | RB | DddA^{N} | N-terminal |
| | | | | GGAGAT | (SEQ | (SEQ | (SEQ | (SEQ | | | | | |
| | | | | (SEQ ID | ID | ID | ID | ID | | | | | |
| | | | | NO: 742) | NO: | NO: | NO: | NO: | | | | | |
| | | | | | 788) | 793) | 804) | 770) | | | | | |
| RB58-Mt-tk | Mt-tk | Mouse | Mt-tk | TATCAC | TSG | QRA | ARG | SKK | ARG | — | RB | DddA^{N} | N- |
| | | | | TATGGA | NLV | HLE | NLR | ALT | NLR | | | | terminal |
| | | | | GAT | R | R | T | E | T | | | | |
| | | | | (SEQ ID | (SEQ | (SEQ | (SEQ | (SEQ | (SEQ | | | | |
| | | | | NO: 743) | ID | ID | ID | ID | ID | | | | |
| | | | | | NO: | NO: | NO: | NO: | NO: | | | | |
| | | | | | 788) | 793) | 804) | 770) | 804) | | | | |
| RB68-Mt-tk | Mt-tk | Mouse | Mt-tk | GCATAT | TSG | QRA | ARG | SKK | ARG | QSG | RB | DddA^{N} | N- |
| | | | | CACTAT | NLV | HLE | NLR | ALT | NLR | DLR | | | terminal |
| | | | | GGAGAT | R | R | T | E | T | R | | | |
| | | | | (SEQ ID | (SEQ | (SEQ | (SEQ | (SEQ | (SEQ | (SEQ | | | |
| | | | | NO: 744) | ID | ID | ID | ID | ID | ID | | | |
| | | | | | NO: | NO: | NO: | NO: | NO: | NO: | | | |
| | | | | | 788) | 793) | 804) | 770) | 804) | 789) | | | |
| LT30-Nd1 | Nd1 | Mouse | Nd1 | ATTTCA | ARG | RSD | HK | — | — | — | LT | DddA^{N} | N- |
| | | | | TAT | NLR | HLT | | | | | | or | terminal |
| | | | | | T | T | QN | | | | | DddA^{C} | |
| | | | | | (SEQ | (SEQ | (SEQ | | | | | | |
| | | | | | ID | ID | ID | | | | | | |
| | | | | | NO: | NO: | NO: | | | | | | |
| | | | | | 804) | 811) | 768) | | | | | | |
| LT31-Nd1 | Nd1 | Mouse | Nd1 | AATTTC | QKS | DNS | TTG | — | — | — | LT | DddA^{N} | N- |
| | | | | ATA | SLI | YLP | NLT | | | | | or | terminal |
| | | | | | A | R | V | | | | | DddA^{C} | |
| | | | | | (SEQ | (SEQ | (SEQ | | | | | | |
| | | | | | ID | ID | ID | | | | | | |
| | | | | | NO: | NO: | NO: | | | | | | |
| | | | | | 765) | 814) | 756) | | | | | | |
| LT33-Nd1 | Nd1 | Mouse | Nd1 | ACAATT | RSD | HK | SPA | — | — | — | LT | DddA^{N} | N- |
| | | | | TCA | HLT | | DLT | | | | | or | terminal |
| | | | | | T | QN | R | | | | | DddA^{C} | |
| | | | | | (SEQ | (SEQ | (SEQ | | | | | | |
| | | | | | ID | ID | ID | | | | | | |
| | | | | | NO: | NO: | NO: | | | | | | |
| | | | | | 811) | 768) | 757) | | | | | | |
| LT34-Nd1 | Nd1 | Mouse | Nd1 | AACAAT | DNS | TTG | DSG | — | — | — | LT | DddA^{N}or | N- |
| | | | | TTC | YLP | NLT | NLR | | | | | | terminal |
| | | | | | R | V | V | | | | | DddA^{C} | |
| | | | | | (SEQ | (SEQ | (SEQ | | | | | | |
| | | | | | ID | ID | ID | | | | | | |
| | | | | | NO: | NO: | NO: | | | | | | |
| | | | | | 814) | 756) | 754) | | | | | | |
| LT36-Nd1 | Nd1 | Mouse | Nd1 | CAAACA | HK | SPA | QSG | — | — | — | LT | DddA^{N} | N- |
| | | | | ATT | | DLT | NLT | | | | | or | terminal |
| | | | | | QN | R | E | | | | | DddA^{C} | |
| | | | | | (SEQ | (SEQ | (SEQ | | | | | | |
| | | | | | ID | ID | ID | | | | | | |
| | | | | | NO: | NO: | NO: | | | | | | |
| | | | | | 768) | 757) | 769) | | | | | | |
| LT37-Nd1 | Nd1 | Mouse | Nd1 | CCAAAC | TTG | DSG | TSH | — | — | — | LT | DddA^{N} | N- |
| | | | | AAT | NLT | NLR | SLT | | | | | or | terminal |
| | | | | | V | V | E | | | | | DddA^{C} | |
| | | | | | (SEQ | (SEQ | (SEQ | | | | | | |
| | | | | | ID | ID | ID | | | | | | |
| | | | | | NO: | NO: | NO: | | | | | | |
| | | | | | 756) | 754) | 773) | | | | | | |
| LT38-Nd1 | Nd1 | Mouse | Nd1 | CCCAAACAA | QSG | QRA | SKK | — | — | — | LT | DddA^{N} | N- |
| | | | | | NLTE | NLRA | HLAE | | | | | or | terminal |
| | | | | | (SEQ | (SEQ | (SEQ | | | | | DddA^{C} | |
| | | | | | ID | ID | ID | | | | | | |
| | | | | | NO: | NO: | NO: 774) | | | | | | |
| | | | | | 769) | 753) | | | | | | | |
| LT39-Nd1 | Nd1 | Mouse | Nd1 | GCCCAAACA | SPA | QSG | DCR | — | — | — | LT | DddA^{N} | N- |
| | | | | | DLT | NLT | DLA | | | | | or | terminal |
| | | | | | R | E | R | | | | | DddA^{C} | |
| | | | | | (SEQ | (SEQ | (SEQ | | | | | | |
| | | | | | ID | ID | ID | | | | | | |
| | | | | | NO: | NO: | NO: | | | | | | |
| | | | | | 757) | 769) | 790) | | | | | | |
| LT310-Nd1 | Nd1 | Mouse | Nd1 | AGCCCAAAC | DSG | TSH | ERS | — | — | — | LT | DddA^{N} | N- |
| | | | | | NLR | SLT | HLR | | | | | or | terminal |
| | | | | | V | E | E | | | | | DddA^{C} | |
| | | | | | (SEQ | (SEQ | (SEQ | | | | | | |
| | | | | | ID | ID | ID | | | | | | |
| | | | | | NO: | NO: | NO: | | | | | | |
| | | | | | 754) | 773) | 762) | | | | | | |
| LT311-Nd1 | Nd1 | Mouse | Nd1 | TAGCCCAAA | QRA | SKK | RED | — | — | — | LT | DddA^{N} | N- |
| | | | | | NLR | HLA | NLH | | | | | or | terminal |
| | | | | | A | E | T | | | | | DddA^{C} | |
| | | | | | (SEQ | (SEQ | (SEQ | | | | | | |
| | | | | | ID | ID | ID | | | | | | |
| | | | | | NO: | NO: | NO: 803) | | | | | | |
| | | | | | 753) | 774) | | | | | | | |
| LB30-Nd1 | Nd1 | Mouse | Nd1 | ATATGAAAT | TTG | QA | QKS | — | — | — | LB | DddA^{N} | Canonical |
| | | | | | NLT | GHL | SLI | | | | | or | |
| | | | | | V | AS | A | | | | | DddA^{C} | |
| | | | | | (SEQ | (SEQ | (SEQ | | | | | | |
| | | | | | ID | ID | ID | | | | | | |
| | | | | | NO: | NO: | NO: | | | | | | |
| | | | | | 756) | 809) | 765) | | | | | | |
| LB31-Nd1 | Nd1 | Mouse | Nd1 | TATGAAATT | HK | QSS | ARG | — | — | — | LB | DddA^{N} | Canonical |
| | | | | | | NLV | NLR | | | | | or | |
| | | | | | QN | R | T | | | | | DddA^{C} | |
| | | | | | (SEQ | (SEQ | (SEQ | | | | | | |
| | | | | | ID | ID | ID | | | | | | |
| | | | | | NO: | NO: | NO: | | | | | | |
| | | | | | 768) | 785) | 804) | | | | | | |
| LB32-Nd1 | Nd1 | Mouse | Nd1 | ATGAAATTG | RKD | QRA | RRD | — | — | — | LB | DddA^{N} | Canonical |
| | | | | | ALR | NLR | ELN | | | | | or | |
| | | | | | G | A | V | | | | | DddA^{C} | |
| | | | | | (SEQ | (SEQ | (SEQ | | | | | | |
| | | | | | ID | ID | ID | | | | | | |
| | | | | | NO: | NO: 753) | NO: 767) | | | | | | |
| | | | | | 815) | | | | | | | | |
| LB33-Nd1 | Nd1 | Mouse | Nd1 | TGAAATTGT | WR | TTG | QA | — | — | — | LB | DddA^{N} | Canonical |
| | | | | | DSL | NLT | GHL | | | | | or | |
| | | | | | LA | V | AS | | | | | DddA^{C} | |
| | | | | | (SEQ | (SEQ | (SEQ | | | | | | |
| | | | | | ID | ID | ID | | | | | | |
| | | | | | NO: | NO: | NO: | | | | | | |
| | | | | | 812) | 756) | 809) | | | | | | |
| LB34-Nd1 | Nd1 | Mouse | Nd1 | GAAATTGTT | TSGSLVR | HKQN | QSSNLVR | — | — | — | LB | DddA^{N} | Canonical |
| | | | | | (SEQ | (SEQ | (SEQ | | | | | or | |
| | | | | | ID | ID | ID | | | | | DddA^{C} | |
| | | | | | NO: | NO: | NO: | | | | | | |
| | | | | | 800) | 768) | 785) | | | | | | |
| LB36-Nd1 | Nd1 | Mouse | Nd1 | AATT | RKD | WR | TTG | — | — | — | LB | DddA^{N} | Canonical |
| | | | | GTTTG | ALR | DSL | NLT | | | | | or | |
| | | | | | G | LA | V | | | | | DddA^{C} | |
| | | | | | (SEQ | (SEQ | (SEQ | | | | | | |
| | | | | | ID | ID | ID | | | | | | |
| | | | | | NO: | NO: | NO: | | | | | | |
| | | | | | 815) | 812) | 756) | | | | | | |
| LB37-Nd1 | Nd1 | Mouse | Nd1 | ATTGT | RSD | TSG | HK | — | — | — | LB | DddA^{N} | Canonical |
| | | | | TTGG | HLT | SLV | | | | | | or | |
| | | | | | T | R | QN | | | | | DddA^{C} | |
| | | | | | (SEQ | (SEQ | (SEQ | | | | | | |
| | | | | | ID | ID | ID | | | | | | |
| | | | | | NO: | NO: | NO: | | | | | | |
| | | | | | 811) | 800) | 768) | | | | | | |
| LB39-Nd1 | Nd1 | Mouse | Nd1 | TGTTT | DPG | RKD | WR | — | — | — | LB | DddA^{N} | Canonical |
| | | | | GGGC | HLV | ALR | DSL | | | | | or | |
| | | | | | R | G | LA | | | | | DddA^{C} | |
| | | | | | (SEQ | (SEQ | (SEQ | | | | | | |
| | | | | | ID | ID | ID | | | | | | |
| | | | | | NO: | NO: | NO: | | | | | | |
| | | | | | 794) | 815) | 812) | | | | | | |
| LB310-Nd1 | Nd1 | Mouse | Nd1 | GTTT | TSG | RSD | TSG | — | — | — | LB | DddA^{N} | Canonical |
| | | | | GGGCT | ELV | HLT | SLV | | | | | or | |
| | | | | | R | T | R | | | | | DddA^{C} | |
| | | | | | (SEQ | (SEQ | (SEQ | | | | | | |
| | | | | | ID | ID | ID | | | | | | |
| | | | | | NO: | NO: | NO: | | | | | | |
| | | | | | 792) | 811) | 800) | | | | | | |
| RT30-Nd1 | Nd1 | Mouse | Nd1 | AAGTA | TSH | QAS | RKD | — | — | — | RT | DddA^{N} | Canonical |
| | | | | ACCA | SLT | NLI | NLK | | | | | or | |
| | | | | | E | S | N | | | | | DddA^{C} | |
| | | | | | (SEQ | (SEQ | (SEQ | | | | | | |
| | | | | | ID | ID | ID | | | | | | |
| | | | | | NO: | NO: | NO: | | | | | | |
| | | | | | 773) | 801) | 755) | | | | | | |
| RT31-Nd1 | Nd1 | Mouse | Nd1 | AGTAA | TSG | DSG | HRT | — | — | — | RT | DddA^{N} | Canonical |
| | | | | CCAT | NLT | NLR | TLT | | | | | or | |
| | | | | | E | V | N | | | | | DddA^{C} | |
| | | | | | (SEQ | (SEQ | (SEQ | | | | | | |
| | | | | | ID | ID | ID | | | | | | |
| | | | | | NO: | NO: | NO: | | | | | | |
| | | | | | 772) | 754) | 764) | | | | | | |
| RT32-Nd1 | Nd1 | Mouse | Nd1 | GTAAC | QKS | DK | QSS | — | — | — | RT | DddA^{N} | Canonical |
| | | | | CATA | SLI | KDL | SLV | | | | | or | |
| | | | | | A | TR | R | | | | | DddA^{C} | |
| | | | | | (SEQ | (SEQ | (SEQ | | | | | | |
| | | | | | ID | ID | ID | | | | | | |
| | | | | | NO: | NO: | NO: | | | | | | |
| | | | | | 765) | 758) | 797) | | | | | | |
| RT33-Nd1 | Nd1 | Mouse | Nd1 | TAACC | RED | TSH | QAS | — | — | — | RT | DddA^{N} | Canonical |
| | | | | ATAG | NLH | SLT | NLI | | | | | or | |
| | | | | | T | E | S | | | | | DddA^{C} | |
| | | | | | (SEQ | (SEQ | (SEQ | | | | | | |
| | | | | | ID | ID | ID | | | | | | |
| | | | | | NO: | NO: | NO: | | | | | | |
| | | | | | 803) | 773) | 801) | | | | | | |
| RT34-Nd1 | Nd1 | Mouse | Nd1 | AACCAT | ERS | TSG | DSG | — | — | — | RT | DddA^{N} | Canonical |
| | | | | AGC | HLR | NLT | NLR | | | | | or | |
| | | | | | E | E | V | | | | | DddA^{C} | |
| | | | | | (SEQ | (SEQ | (SEQ | | | | | | |
| | | | | | ID | ID | ID | | | | | | |
| | | | | | NO: | NO: | NO: | | | | | | |
| | | | | | 762) | 772) | 754) | | | | | | |
| RT35-Nd1 | Nd1 | Mouse | Nd1 | ACCAT | TSG | QKS | DK | — | — | — | RT | DddA^{N} | Canonical |
| | | | | AGCT | ELV | SLI | KDL | | | | | or | |
| | | | | | R | A | TR | | | | | DddA^{C} | |
| | | | | | (SEQ | (SEQ | (SEQ | | | | | | |
| | | | | | ID | ID | ID | | | | | | |
| | | | | | NO: | NO: | NO: | | | | | | |
| | | | | | 792) | 765) | 758) | | | | | | |
| RT36-Nd1 | Nd1 | Mouse | Nd1 | CCATA | QNS | RED | TSH | — | — | — | RT | DddA^{N} | Canonical |
| | | | | GCTA | TLT | NLH | SLT | | | | | or | |
| | | | | | E | T | E | | | | | DddA^{C} | |
| | | | | | (SEQ | (SEQ | (SEQ | | | | | | |
| | | | | | ID | ID | ID | | | | | | |
| | | | | | NO: | NO: | NO: | | | | | | |
| | | | | | 781) | 803) | 773) | | | | | | |
| RT37-Nd1 | Nd1 | Mouse | Nd1 | CATAG | ARG | ERS | TSG | — | — | — | RT | DddA^{N} | Canonical |
| | | | | CTAT | NLR | HLR | NLT | | | | | or | |
| | | | | | T | E | E | | | | | DddA^{C} | |
| | | | | | (SEQ | (SEQ | (SEQ | | | | | | |
| | | | | | ID | ID | ID | | | | | | |
| | | | | | NO: | NO: | NO: | | | | | | |
| | | | | | 804) | 762) | 772) | | | | | | |
| RT38-Nd1 | Nd1 | Mouse | Nd1 | ATAGC | HK | TSG | QKS | — | — | — | RT | DddA^{N} | Canonical |
| | | | | TATT | | ELV | SLI | | | | | or | |
| | | | | | QN | R | A | | | | | DddA^{C} | |
| | | | | | (SEQ | (SEQ | (SEQ | | | | | | |
| | | | | | ID | ID | ID | | | | | | |
| | | | | | NO: | NO: | NO: | | | | | | |
| | | | | | 768) | 792) | 765) | | | | | | |
| RT39-Nd1 | Nd1 | Mouse | Nd1 | TAGCT | QK | QNS | RED | — | — | — | RT | DddA^{N} | Canonical |
| | | | | ATTA | WP | TLT | NLH | | | | | or | |
| | | | | | RDS | E | T | | | | | DddA^{C} | |
| | | | | | (SEQ | (SEQ | (SEQ | | | | | | |
| | | | | | ID | ID | ID | | | | | | |
| | | | | | NO: | NO: | NO: | | | | | | |
| | | | | | 813) | 781) | 803) | | | | | | |
| RT310-Nd1 | Nd1 | Mouse | Nd1 | AGCTA | ARG | ARG | ERS | — | — | — | RT | DddA^{N} | Canonical |
| | | | | TTAT | NLR | NLR | HLR | | | | | or | |
| | | | | | T | T | E | | | | | DddA^{C} | |
| | | | | | (SEQ | (SEQ | (SEQ | | | | | | |
| | | | | | ID | ID | ID | | | | | | |
| | | | | | NO: | NO: | NO: | | | | | | |
| | | | | | 804) | 804) | 762) | | | | | | |
| RT311-Nd1 | Nd1 | Mouse | Nd1 | GCTAT | RRS | HK | TSG | — | — | — | RT | DddA^{N} | Canonical |
| | | | | TATC | ACR | | ELV | | | | | or | |
| | | | | | R | QN | R | | | | | DddA^{C} | |
| | | | | | (SEQ | (SEQ | (SEQ | | | | | | |
| | | | | | ID | ID | ID | | | | | | |
| | | | | | NO: | NO: | NO: | | | | | | |
| | | | | | 766) | 768) | 792) | | | | | | |
| RB30-Nd1 | Nd1 | Mouse | Nd1 | TGGTT | TTGALT | QKWPRDS | RSDHLTT | — | — | — | RB | DddA^{N} | N-terminal |
| | | | | ACTT | E (SEQ | (SEQ | (SEQ | | | | | or | |
| | | | | | ID | ID | ID | | | | | | |
| | | | | | NO: | NO: | NO: | | | | | | |
| | | | | | 784) | 813) | 811) | | | | | | |
| RB31-Nd1 | Nd1 | Mouse | Nd1 | ATGGT | THL | TSG | RRD | | — | — | RB | DddA^{N} | N- |
| | | | | TACT | DLI | SLV | ELN | | | | | or | terminal |
| | | | | | R | R | V | | | | | DddA^{C} | |
| | | | | | (SEQ | (SEQ | (SEQ | | | | | | |
| | | | | | ID | ID | ID | | | | | | |
| | | | | | NO: | NO: | NO: | | | | | | |
| | | | | | 760) | 800) | 767) | | | | | | |
| RB32-Nd1 | Nd1 | Mouse | Nd1 | TATGG | SRG | TSG | ARG | — | — | — | RB | DddA^{N} | N- |
| | | | | TTAC | NLK | HLV | NLR | | | | | or | terminal |
| | | | | | S | R | T | | | | | DddA^{C} | |
| | | | | | (SEQ | (SEQ | (SEQ | | | | | | |
| | | | | | ID | ID | ID | | | | | | |
| | | | | | NO: | NO: | NO: | | | | | | |
| | | | | | 802) | 796) | 804) | | | | | | |
| RB33-Nd1 | Nd1 | Mouse | Nd1 | CTATG | QK | RSD | QNS | — | — | — | RB | DddA^{N} | N- |
| | | | | GTTA | WP | HLT | TLT | | | | | or | terminal |
| | | | | | RDS | T | E | | | | | DddA^{C} | |
| | | | | | (SEQ | (SEQ | (SEQ | | | | | | |
| | | | | | ID | ID | ID | | | | | | |
| | | | | | NO: | NO: | NO: | | | | | | |
| | | | | | 813) | 811) | 781) | | | | | | |
| RB34-Nd1 | Nd1 | Mouse | Nd1 | GCTAT | TSG | RRD | TSG | — | — | — | RB | DddA^{N} | N- |
| | | | | GGTT | SLV | ELN | ELV | | | | | or | terminal |
| | | | | | R | V | R | | | | | DddA^{C} | |
| | | | | | (SEQ | (SEQ | (SEQ | | | | | | |
| | | | | | ID | ID | ID | | | | | | |
| | | | | | NO: | NO: | NO: | | | | | | |
| | | | | | 800) | 767) | 792) | | | | | | |
| RB35-Nd1 | Nd1 | Mouse | Nd1 | AGCTA | TSG | ARG | ERS | — | — | — | RB | DddA^{N} | N- |
| | | | | TGGT | HLV | NLR | HLR | | | | | or | terminal |
| | | | | | R | T | E | | | | | DddA^{C} | |
| | | | | | (SEQ | (SEQ | (SEQ | | | | | | |
| | | | | | ID | ID | ID | | | | | | |
| | | | | | NO: | NO: | NO: | | | | | | |
| | | | | | 796) | 804) | 762) | | | | | | |
| RB36-Nd1 | Nd1 | Mouse | Nd1 | TAGCT | RSD | QNS | RED | — | — | — | RB | DddA^{N} | N- |
| | | | | ATGG | HLT | TLT | NLH | | | | | or | terminal |
| | | | | | T | E | T | | | | | DddA^{C} | |
| | | | | | (SEQ | (SEQ | (SEQ | | | | | | |
| | | | | | ID | ID | ID | | | | | | |
| | | | | | NO: | NO: | NO: | | | | | | |
| | | | | | 811) | 781) | 803) | | | | | | |
| RB37-Nd1 | Nd1 | Mouse | Nd1 | ATAG | RRD | TSG | QKS | — | — | — | RB | DddA^{N} | N- |
| | | | | CTATG | ELN | ELV | SLI | | | | | or | terminal |
| | | | | | V | R | A | | | | | DddA^{C} | |
| | | | | | (SEQ | (SEQ | (SEQ | | | | | | |
| | | | | | ID | ID | ID | | | | | | |
| | | | | | NO: | NO: | NO: | | | | | | |
| | | | | | 767) | 792) | 765) | | | | | | |
| RB38-Nd1 | Nd1 | Mouse | Nd1 | AATA | ARG | ERS | TTG | — | — | — | RB | DddA^{N} | N-terminal |
| | | | | GCTAT | NLR | HLR | NLT | | | | | or | |
| | | | | | T | E | V | | | | | DddA^{C} | |
| | | | | | (SEQ | (SEQ | (SEQ | | | | | | |
| | | | | | ID | ID | ID | | | | | | |
| | | | | | NO: | NO: | NO: | | | | | | |
| | | | | | 804) | 762) | 756) | | | | | | |
| RB39-Nd1 | Nd1 | Mouse | Nd1 | TAATAG | QNS | RED | QAS | — | — | — | RB | DddA^{N} | N- |
| | | | | CTA | TLT | NLH | NLI | | | | | or | terminal |
| | | | | | E | T | S | | | | | DddA^{C} | |
| | | | | | (SEQ | (SEQ | (SEQ | | | | | | |
| | | | | | ID | ID | ID | | | | | | |
| | | | | | NO: | NO: | NO: | | | | | | |
| | | | | | 781) | 803) | 801) | | | | | | |
| RB310-Nd1 | Nd1 | Mouse | Nd1 | ATAATA | TSG | QKS | QKS | — | — | — | RB | DddA^{N} | N- |
| | | | | GCT | ELV | SLI | SLI | | | | | or | terminal |
| | | | | | R | A | A | | | | | DddA^{C} | |
| | | | | | (SEQ | (SEQ | (SEQ | | | | | | |
| | | | | | ID | ID | ID | | | | | | |
| | | | | | NO: | NO: | NO: | | | | | | |
| | | | | | 792) | 765) | 765) | | | | | | |
| RB311-Nd1 | Nd1 | Mouse | Nd1 | GATAAT | ERS | TTG | TSG | — | — | — | RB | DddA^{N} | N- |
| | | | | AGC | HLR | NLT | NLV | | | | | or | terminal |
| | | | | | E | V | R | | | | | DddA^{C} | |
| | | | | | (SEQ | (SEQ | (SEQ | | | | | | |
| | | | | | ID | ID | ID | | | | | | |
| | | | | | NO: | NO: | NO: | | | | | | |
| | | | | | 762) | 756) | 788) | | | | | | |
| RB312-Nd1 | Nd1 | Mouse | Nd1 | GGATAA | RED | QAS | QRA | — | — | — | RB | DddA^{N} | N- |
| | | | | TAG | NLH | NLI | HLE | | | | | or | terminal |
| | | | | | T | S | R | | | | | DddA^{C} | |
| | | | | | (SEQ | (SEQ | (SEQ | | | | | | |
| | | | | | ID | ID | ID | | | | | | |
| | | | | | NO: | NO: | NO: | | | | | | |
| | | | | | 803) | 801) | 793) | | | | | | |
| LB410-Nd1 | Nd1 | Mouse | Nd1 | GTTTGG | RTD | TSG | RSD | TSG | — | — | LB | DddA^{C} | Canonical |
| | | | | GCTACG | TLR | ELV | HLT | SLV | | | | | |
| | | | | (SEQ ID | D | R | T | R | | | | | |
| | | | | NO: 745) | (SEQ | (SEQ | (SEQ | (SEQ | | | | | |
| | | | | | ID | ID | ID | ID | | | | | |
| | | | | | NO: | NO: | NO: | NO: | | | | | |
| | | | | | 759) | 792) | 811) | 800) | | | | | |
| LB510-Nd1 | Nd1 | Mouse | Nd1 | GTTTGG | TSG | RTD | TSG | RSD | TSG | — | LB | DddA^{C} | Canonical |
| | | | | GCTACG | ELV | TLR | ELV | HLT | SLV | | | | |
| | | | | GCT | R | D | R | T | R | | | | |
| | | | | (SEQ ID | (SEQ | (SEQ | (SEQ | (SEQ | (SEQ | | | | |
| | | | | NO: 746) | ID | ID | ID | ID | ID | | | | |
| | | | | | NO: | NO: | NO: | NO: | NO: | | | | |
| | | | | | 792) | 759) | 792) | 811) | 800) | | | | |
| RT55-Nd1 | Nd1 | Mouse | Nd1 | ACCATA | RRS | HK | TSG | QKS | DK | — | RT | DddA^{N} | Canonical |
| | | | | GCTATT | ACR | | ELV | SLI | KDL | | | | |
| | | | | ATC | R | QN | R | A | TR | | | | |
| | | | | (SEQ ID | (SEQ | (SEQ | (SEQ | (SEQ | (SEQ | | | | |
| | | | | NO: 747) | ID | ID | ID | ID | ID | | | | |
| | | | | | NO: | NO: | NO: | NO: | NO: | | | | |
| | | | | | 766) | 768) | 792) | 765) | 758) | | | | |
| RT65-Nd1 | Nd1 | Mouse | Nd1 | ACCATA | TTG | RRS | HK | TSG | QKS | DKKDL | RT | DddA^{N} | Canonical |
| | | | | GCTATT | ALT | ACR | | ELV | SLI | | | | |
| | | | | ATCCTT | E | R | QN | R | A | TR | | | |
| | | | | (SEQ ID | (SEQ | (SEQ | (SEQ | (SEQ | (SEQ | (SEQ | | | |
| | | | | NO: 748) | ID | ID | ID | ID | ID | ID | | | |
| | | | | | NO: | NO: | NO: | NO: | NO: | NO: | | | |
| | | | | | 784) | 766) | 768) | 792) | 765) | 758) | | | |
| RB44-Nd1 | Nd1 | Mouse | Nd1 | ATAGCT | TSG | RRD | TSG | QKS | — | — | RB | DddA^{N} | N- |
| | | | | ATGGTT | SLV | ELN | ELV | SLI | | | | | terminal |
| | | | | (SEQ ID | R | V | R | A | | | | | |
| | | | | NO: 749) | (SEQ | (SEQ | (SEQ | (SEQ | | | | | |
| | | | | | ID | ID | ID | ID | | | | | |
| | | | | | NO: | NO: | NO: | NO: | | | | | |
| | | | | | 800) | 767) | 792) | 765) | | | | | |
| RB54-Nd1 | Nd1 | Mouse | Nd1 | ATAATA | TSG | RRD | TSG | QKS | QKS | — | RB | DddA^{N} | N- |
| | | | | GCTATG | SLV | ELN | ELV | SLI | SLI | | | | terminal |
| | | | | GTT | R | V | R | A | A | | | | |
| | | | | (SEQ ID | (SEQ | (SEQ | (SEQ | (SEQ | (SEQ | | | | |
| | | | | NO: 750) | ID | ID | ID | ID | ID | | | | |
| | | | | | NO: | NO: | NO: | NO: | NO: | | | | |
| | | | | | 800) | 767) | 792) | 765) | 765) | | | | |
| RB64-Nd1 | Nd1 | Mouse | Nd1 | AGGATA | TSG | RRD | TSG | QKS | QKS | RSD | RB | DddA^{N} | N- |
| | | | | ATAGCT | SLV | ELN | ELV | SLI | SLI | HLT | | | terminal |
| | | | | ATGGTT | R | V | R | A | A | N | | | |
| | | | | (SEQ ID | (SEQ | (SEQ | (SEQ | (SEQ | (SEQ | (SEQ | | | |
| | | | | NO: 751) | ID | ID | ID | ID | ID | ID | | | |
| | | | | | NO: | NO: | NO: | NO: | NO: | NO: | | | |
| | | | | | 800) | 767) | 792) | 765) | 765) | 763) | | | |
| RB47-Nd1 | Nd1 | Mouse | Nd1 | ATAATA | RRD | TSG | QKS | QKS | — | — | RB | DddA^{N} | N- |
| | | | | GCTATG | ELN | ELV | SLI | SLI | | | | | terminal |
| | | | | (SEQ ID | V | R | A | A | | | | | |
| | | | | NO: 752) | (SEQ | (SEQ | (SEQ | (SEQ | | | | | |
| | | | | | ID | ID | ID | ID | | | | | |
| | | | | | NO: | NO: | NO: | NO: | | | | | |
| | | | | | 767) | 792) | 765) | 765) | | | | | |
| G35-R6c | ND6 | Human | ND62 | GTTGAG | DPG | RSD | TSG | — | — | — | LB | DddA^{C} | Canonical |
| | | | | GTC | ALV | NLV | SLV | | | | | | |
| | | | | | R | R | R | | | | | | |
| | | | | | (SEQ | (SEQ | (SEQ | | | | | | |
| | | | | | ID | ID | ID | | | | | | |
| | | | | | NO: | NO: | NO: | | | | | | |
| | | | | | 798) | 787) | 800) | | | | | | |
| G28-R6c | ND6 | Human | ND62 | GAGGTC | RKD | DPG | RSD | — | — | — | LB | DddA^{C} | Canonical |
| | | | | TTG | ALR | ALV | NLV | | | | | | |
| | | | | | G | R | R | | | | | | |
| | | | | | (SEQ | (SEQ | (SEQ | | | | | | |
| | | | | | ID | ID | ID | | | | | | |
| | | | | | NO: | NO: | NO: | | | | | | |
| | | | | | 815) | 798) | 787) | | | | | | |

TABLE 8
Nuclear ZFs

| | | | Target DNA | | | | | | | Target | | |
|---|---|---|---|---|---|---|---|---|---|---|---|---|
| | | Amp- | sequence | | | | | | | DNA | DddA | Archi- |
| Name | Species | licon | (5' to 3') | ZF1 | ZF2 | ZF3 | ZF4 | ZF5 | ZF6 | strand | split | tecture |
| 3xG22- | Human | COL5A1 | GAGTAGG | RSD | RED | RSD | — | — | — | LB | DddA^{N} | Canon- |
| COL5A1 | | | AG | NLV | NLH | NLV | | | | | | ical |
| | | | | R | T | R | | | | | | |
| | | | | (SEQ | (SEQ | (SEQ | | | | | | |
| | | | | ID | ID | ID | | | | | | |
| | | | | NO: | NO: | NO: | | | | | | |
| | | | | 787) | 803) | 787) | | | | | | |
| 4xG22- | Human | COL5A1 | GAGTAGG | QSG | RSD | RED | RSD | — | — | LB | DddA^{N} | Canon- |
| COL5A1 | | | AGGCA | DLR | NLV | NLH | NLV | | | | | ical |
| | | | (SEQ ID | R | R | T | R | | | | | |
| | | | NO: 892) | (SEQ | (SEQ | (SEQ | (SEQ | | | | | |
| | | | | ID | ID | ID | ID | | | | | |
| | | | | NO: | NO: | NO: | NO: | | | | | |
| | | | | 789) | 787) | 803) | 787) | | | | | |
| 5xG22- | Human | COL5A1 | GAGTAGG | TTG | QSG | RSD | RED | RSD | — | LB | DddA^{N} | Canon- |
| COL5A1 | | | AGGCAAA | NLT | DLR | NLV | NLH | NLV | | | | ical |
| | | | T (SEQ ID | V | R | R | T | R | | | | |
| | | | NO: 901) | (SEQ | (SEQ | (SEQ | (SEQ | (SEQ | | | | |
| | | | | ID | ID | ID | ID | ID | | | | |
| | | | | NO: | NO: | NO: | NO: | NO: | | | | |
| | | | | 756) | 789) | 787) | 803) | 787) | | | | |
| 6xG22- | Human | COL5A1 | GAGTAGG | QRH | TTG | QSG | RSD | RED | RSD | LB | DddA^{N} | Canon- |
| COL5A1 | | | AGGCAAA | HLV | NLT | DLR | NLV | NLH | NLV | | | ical |
| | | | TCTC (SEQ | E | V | R | R | T | R | | | |
| | | | ID NO: | (SEQ | (SEQ | (SEQ | (SEQ | (SEQ | (SEQ | | | |
| | | | 910) | ID | ID | ID | ID | ID | ID | | | |
| | | | | NO: | NO: | NO: | NO: | NO: | NO: | | | |
| | | | | 782) | 756) | 789) | 787) | 803) | 787) | | | |
| 3xG34- | Human | COL5A1 | GTGGAGG | DCR | RSD | RSD | — | — | — | RT | DddA^{C} | Canon- |
| COL5A1 | | | CC | DLA | NLV | ELV | | | | | | ical |
| | | | | R | R | R | | | | | | |
| | | | | (SEQ | (SEQ | (SEQ | | | | | | |
| | | | | ID | ID | ID | | | | | | |
| | | | | NO: | NO: | NO: | | | | | | |
| | | | | 790) | 787) | 799) | | | | | | |
| 4xG34- | Human | COL5A1 | GTGGAGG | RTD | DCR | RSD | RSD | — | — | RT | DddA^{C} | Canon- |
| COL5A1 | | | CCACG | TLR | DLA | NLV | ELV | | | | | ical |
| | | | (SEQ ID | D | R | R | R | | | | | |
| | | | NO: 893) | (SEQ | (SEQ | (SEQ | (SEQ | | | | | |
| | | | | ID | ID | ID | ID | | | | | |
| | | | | NO: | NO: | NO: | NO: | | | | | |
| | | | | 759) | 790) | 787) | 799) | | | | | |
| 5xG34- | Human | COL5A1 | GTGGAGG | TSG | RTD | DCR | RSD | RSD | — | RT | DddA^{C} | Canon- |
| COL5A1 | | | CCACGGCT | ELV | TLR | DLA | NLV | ELV | | | | ical |
| | | | (SEQ ID | R | D | R | R | R | | | | |
| | | | NO: 902) | (SEQ | (SEQ | (SEQ | (SEC | (SEC | | | | |
| | | | | ID | ID | ID | ID | ID | | | | |
| | | | | NO: | NO: | NO: | NO: | NO: | | | | |
| | | | | 792) | 759) | 790) | 787) | 799) | | | | |
| 6xG34- | Human | COL5A1 | GTGGAGG | RND | TSG | RTD | DCR | RSD | RSD | RT | DddA^{C} | Canon- |
| COL5A1 | | | CCACGGCT | ALT | ELV | TLR | DLA | NLV | ELV | | | ical |
| | | | CTG (SEQ | E | R | D | R | R | R | | | |
| | | | ID NO: | (SEQ | (SEQ | (SEQ | (SEQ | (SEQ | (SEQ | | | |
| | | | 911) | ID | ID | ID | ID | ID | ID | | | |
| | | | | NO: | NO: | NO: | NO: | NO: | NO: | | | |
| | | | | 783) | 792) | 759) | 790) | 787) | 799) | | | |
| 3xG22- | Human | DCA | GAGTAGG | RSD | RED | RSD | — | — | — | LB | DddA^{N} | Canon- |
| DCAF8 | | F8L2 | AG | NLV | NLH | NLV | | | | | | ical |
| L2 | | | | R | T | R | | | | | | |
| | | | | (SEQ | (SEQ | (SEQ | | | | | | |
| | | | | ID | ID | ID | | | | | | |
| | | | | NO: | NO: | NO: | | | | | | |
| | | | | 787) | 803) | 787) | | | | | | |
| 4xG22- | Human | DCA | GAGTAGG | WRD | RSD | RED | RSD | — | — | LB | DddA^{N} | Canon- |
| DCAF8 | | F8L2 | AGTGT | SLL | NLV | NLH | NLV | | | | | ical |
| L2 | | | (SEQ ID | A | R | T | R | | | | | |
| | | | NO: 894) | (SEQ | (SEQ | (SEQ | (SEQ | | | | | |
| | | | | ID | ID | ID | ID | | | | | |
| | | | | NO: | NO: | NO: | NO: | | | | | |
| | | | | 812) | 787) | 803) | 787) | | | | | |
| 5xG22- | Human | DCA | GAGTAGG | RAD | WRD | RSD | RED | RSD | — | LB | DddA^{N} | Canon- |
| DCAF8 | | F8L2 | AGTGTCAG | NLT | SLL | NLV | NLH | NLV | | | | ical |
| L2 | | | (SEQ ID | E | A | R | T | R | | | | |
| | | | NO: 903) | (SEQ | (SEQ | (SEQ | (SEQ | (SEQ | | | | |
| | | | | ID | ID | ID | ID | ID | | | | |
| | | | | NO: | NO: | NO: | NO: | NO: | | | | |
| | | | | 771) | 812) | 787) | 803) | 787) | | | | |
| 6xG22- | Human | DCA | GAGTAGG | TSG | RAD | WRD | RSD | RED | RSD | LB | DddA^{N} | Canon- |
| DCAF8 | | F8L2 | AGTGTCAG | SLV | NLT | SLL | NLV | NLH | NLV | | | ical |
| L2 | | | GTT (SEQ | R | E | A | R | T | R | | | |
| | | | ID NO: | (SEQ | (SEQ | (SEQ | (SEQ | (SEQ | (SEQ | | | |
| | | | 912) | ID | ID | ID | ID | ID | ID | | | |
| | | | | NO: | NO: | NO: | NO: | NO: | NO: | | | |
| | | | | 800) | 771) | 812) | 787) | 803) | 787) | | | |
| 3xG34- | Human | DCA | GTGGAGG | DCR | RSD | RSD | — | — | — | RT | DddA^{C} | Canon- |
| DCAF8 | | F8L2 | CC | DLA | NLV | ELV | | | | | | ical |
| L2 | | | | R | R | R | | | | | | |
| | | | | (SEQ | (SEQ | (SEQ | | | | | | |
| | | | | ID | ID | ID | | | | | | |
| | | | | NO: | NO: | NO: | | | | | | |
| | | | | 790) | 787) | 799) | | | | | | |
| 4xG34- | Human | DCA | GTGGAGG | RED | DCR | RSD | RSD | — | — | RT | DddA^{C} | Canon- |
| DCAF8 | | F8L2 | CCTAG | NLH | DLA | NLV | ELV | | | | | ical |
| L2 | | | (SEQ ID | T | R | R | R | | | | | |
| | | | NO: 895) | (SEQ | (SEQ | (SEQ | (SEQ | | | | | |
| | | | | ID | ID | ID | ID | | | | | |
| | | | | NO: | NO: | NO: | NO: | | | | | |
| | | | | 803) | 790) | 787) | 799) | | | | | |
| 5xG34- | Human | DCA | GTGGAGG | SPA | RED | DCR | RSD | RSD | — | RT | DddA^{C} | Canon- |
| DCAF8 | | F8L2 | CCTAGACA | DLT | NLH | DLA | NLV | ELV | | | | ical |
| L2 | | | (SEQ ID | R | T | R | R | R | | | | |
| | | | NO: 904) | (SEQ | (SEQ | (SEQ | (SEQ | (SEQ | | | | |
| | | | | ID | ID | ID | ID | ID | | | | |
| | | | | NO: | NO: | NO: | NO: | NO: | | | | |
| | | | | 757) | 803) | 790) | 787) | 799) | | | | |
| 6xG34- | Human | DCA | GTGGAGG | RSD | SPA | RED | DCR | RSD | RSD | RT | DddA^{C} | Canon- |
| DCAF8 | | F8L2 | CCTAGACA | DLV | DLT | NLH | DLA | NLV | ELV | | | ical |
| L2 | | | GCG (SEQ | R | R | T | R | R | R | | | |
| | | | ID NO: | (SEQ | (SEQ | (SEQ | (SEQ | (SEQ | (SEQ | | | |
| | | | 913) | ID | ID | ID | ID | ID | ID | | | |
| | | | | NO: | NO: | NO: | NO: | NO: | NO: | | | |
| | | | | 791) | 757) | 803) | 790) | 787) | 799) | | | |
| 3xG28- | Human | EMI | GAGGTCTT | RKD | DPG | RSD | — | — | — | LB | DddA^{C} | Canon- |
| EMILIN | | LIN2 | G | ALR | ALV | NLV | | | | | | ical |
| 2 | | | | G | R | R | | | | | | |
| | | | | (SEQ | (SEQ | (SEQ | | | | | | |
| | | | | ID | ID | ID | | | | | | |
| | | | | NO: | NO: | NO: | | | | | | |
| | | | | 815) | 798) | 787) | | | | | | |
| 4xG28- | Human | EMI | GAGGTCTT | QSSS | RKD | DPG | RSD | — | — | LB | DddA^{C} | Canon- |
| EMILIN | | LIN2 | GGTA (SEQ | LVR | ALR | ALV | NLV | | | | | ical |
| 2 | | | ID NO: | (SEQ | G | R | R | | | | | |
| | | | 896) | ID | (SEQ | (SEQ | (SEQ | | | | | |
| | | | | NO: | ID | ID | ID | | | | | |
| | | | | 797) | NO: | NO: | NO: | | | | | |
| | | | | | 815) | 798) | 787) | | | | | |
| 5xG28- | Human | EMI | GAGGTCTT | HRT | QSSS | RKD | DPG | RSD | — | LB | DddA^{C} | Canon- |
| EMILIN | | LIN2 | GGTAAGT | TLT | LVR | ALR | ALV | NLV | | | | ical |
| 2 | | | (SEQ ID | N | (SEQ | G | R | R | | | | |
| | | | NO: 905) | (SEQ | ID | (SEQ | (SEQ | (SEQ | | | | |
| | | | | ID | NO: | ID | ID | ID | | | | |
| | | | | NO: | 797) | NO: | NO: | NO: | | | | |
| | | | | 764) | | 815) | 798) | 787) | | | | |
| 6xG28- | Human | EMI | GAGGTCTT | TSG | HRT | QSSS | RKD | DPG | RSD | LB | DddA^{C} | Canon- |
| EMILIN | | LIN2 | GGTAAGTG | SLV | TLT | LVR | ALR | ALV | NLV | | | ical |
| 2 | | | TT (SEQ ID | R | N | (SEQ | G | R | R | | | |
| | | | NO: 914) | (SEQ | (SEQ | ID | (SEQ | (SEQ | (SEQ | | | |
| | | | | ID | ID | NO: | ID | ID | ID | | | |
| | | | | NO: | NO: | 797) | NO: | NO: | NO: | | | |
| | | | | 800) | 764) | | 815) | 798) | 787) | | | |
| 3xG35- | Human | EMI | GTTGAGGT | DPG | RSD | TSG | — | — | — | LB | DddA^{C} | Canon- |
| EMILIN | | LIN2 | C | ALV | NLV | SLV | | | | | | ical |
| 2 | | | | R | R | R | | | | | | |
| | | | | (SEQ | (SEQ | (SEQ | | | | | | |
| | | | | ID | ID | ID | | | | | | |
| | | | | NO: | NO: | NO: | | | | | | |
| | | | | 798) | 787) | 800) | | | | | | |
| 4xG35- | Human | EMI | GTTGAGGT | RKD | DPG | RSD | TSG | — | — | LB | DddA^{C} | Canon- |
| EMILIN | | LIN2 | CTTG (SEQ | ALR | ALV | NLV | SLV | | | | | ical |
| 2 | | | ID NO: | G | R | R | R | | | | | |
| | | | 897) | (SEQ | (SEQ | (SEQ | (SEQ | | | | | |
| | | | | ID | ID | ID | ID | | | | | |
| | | | | NO: | NO: | NO: | NO: | | | | | |
| | | | | 815) | 798) | 787) | 800) | | | | | |
| 5xG35- | Human | EMI | GTTGAGGT | QSSS | RKD | DPG | RSD | TSG | — | LB | DddA^{C} | Canon- |
| EMILIN | | LIN2 | CTTGGTA | LVR | ALR | ALV | NLV | SLV | | | | ical |
| 2 | | | (SEQ ID | (SEQ | G | R | R | R | | | | |
| | | | NO: 906) | ID | (SEQ | (SEQ | (SEQ | (SEQ | | | | |
| | | | | NO: | ID | ID | ID | ID | | | | |
| | | | | 797) | NO: | NO: | NO: | NO: | | | | |
| | | | | | 815) | 798) | 787) | 800) | | | | |
| 6xG35- | Human | EMI | GTTGAGGT | HRT | QSSS | RKD | DPG | RSD | TSG | LB | DddA^{C} | Canon- |
| EMILIN | | LIN2 | CTTGGTAA | TLT | LVR | ALR | ALV | NLV | SLV | | | ical |
| 2 | | | GT (SEQ ID | N | (SEQ | G | R | R | R | | | |
| | | | NO: 915) | (SEQ | ID | (SEQ | (SEQ | (SEQ | (SEQ | | | |
| | | | | ID | NO: | ID | ID | ID | ID | | | |
| | | | | NO: | 797) | NO: | NO: | NO: | NO: | | | |
| | | | | 764) | | 815) | 798) | 787) | 800) | | | |
| 3xG212- | Human | EMI | GAGGCAT | RSD | QSG | RSD | — | — | — | RB | DddA^{N} | N- |
| EMILIN | | LIN2 | GG | HLT | DLR | NLV | | | | | | terminal |
| 2 | | | | T | R | R | | | | | | |
| | | | | (SEQ | (SEQ | (SEQ | | | | | | |
| | | | | ID | ID | ID | | | | | | |
| | | | | NO: | NO: | NO: | | | | | | |
| | | | | 811) | 789) | 787) | | | | | | |
| 4xG212- | Human | EMI | CCTGAGGC | RSD | QSG | RSD | TKN | — | — | RB | DddA^{N} | N- |
| EMILIN | | LIN2 | ATGG (SEQ | HLT | DLR | NLV | SLT | | | | | terminal |
| 2 | | | ID NO: | T | R | R | E | | | | | |
| | | | 898) | (SEQ | (SEQ | (SEQ | (SEQ | | | | | |
| | | | | ID | ID | ID | ID | | | | | |
| | | | | NO: | NO: | NO: | NO: | | | | | |
| | | | | 811) | 789) | 787) | 776) | | | | | |
| 5xG212- | Human | EMI | TATCCTGA | RSD | QSG | RSD | TKN | ARG | — | RB | DddA^{N} | N- |
| EMILIN | | LIN2 | GGCATGG | HLT | DLR | NLV | SLT | NLR | | | | terminal |
| 2 | | | (SEQ ID | T | R | R | E | T | | | | |
| | | | NO: 907) | (SEQ | (SEQ | (SEQ | (SEQ | (SEQ | | | | |
| | | | | ID | ID | ID | ID | ID | | | | |
| | | | | NO: | NO: | NO: | NO: | NO: | | | | |
| | | | | 811) | 789) | 787) | 776) | 804) | | | | |
| 6xG212- | Human | EMI | GAGTATCC | RSD | QSG | RSD | TKN | ARG | RSD | RB | DddA^{N} | N- |
| EMILIN | | LIN2 | TGAGGCAT | HLT | DLR | NLV | SLT | NLR | NLV | | | terminal |
| 2 | | | GG (SEQ ID | T | R | R | E | T | R | | | |
| | | | NO: 916) | (SEQ | (SEQ | (SEQ | (SEQ | (SEQ | (SEQ | | | |
| | | | | ID | ID | ID | ID | ID | ID | | | |
| | | | | NO: | NO: | NO: | NO: | NO: | NO: | | | |
| | | | | 811) | 789) | 787) | 776) | 804) | 787) | | | |
| 3xG24- | Human | TRA | GAGGCTCC | TSH | TSG | RSD | — | — | — | LB | DddA^{C} | Canon- |
| TRAM1 | | M1L1 | A | SLTE | ELV | NLV | | | | | | ical |
| L1 | | | | (SEQ | R | R | | | | | | |
| | | | | ID | (SEQ | (SEQ | | | | | | |
| | | | | NO: | ID | ID | | | | | | |
| | | | | 773) | NO: | NO: | | | | | | |
| | | | | | 792) | 787) | | | | | | |
| 4xG24- | Human | TRA | GAGGCTCC | ERS | TSH | TSG | RSD | — | — | LB | DddA^{C} | Canon- |
| TRAM1 | | M1L1 | AAGC (SEQ | HLR | SLTE | ELV | NLV | | | | | ical |
| L1 | | | ID NO: | E | (SEQ | R | R | | | | | |
| | | | 899) | (SEQ | ID | (SEQ | (SEQ | | | | | |
| | | | | ID | NO: | ID | ID | | | | | |
| | | | | NO: | 773) | NO: | NO: | | | | | |
| | | | | 762) | | 792) | 787) | | | | | |
| 5xG24- | Human | TRA | GAGGCTCC | QRA | ERS | TSH | TSG | RSD | — | LB | DddA^{C} | Canon- |
| TRAM1 | | M1L1 | AAGCAAA | NLR | HLR | SLTE | ELV | NLV | | | | ical |
| L1 | | | (SEQ ID | A | E | (SEQ | R | R | | | | |
| | | | NO: 908) | (SEQ | (SEQ | ID | (SEQ | (SEQ | | | | |
| | | | | ID | ID | NO: | ID | ID | | | | |
| | | | | NO: | NO: | 773) | NO: | NO: | | | | |
| | | | | 753) | 762) | | 792) | 787) | | | | |
| 6xG24- | Human | TRA | GAGGCTCC | HKN | QRA | ERS | TSH | TSG | RSD | LB | DddA^{C} | Canon- |
| TRAM1 | | M1L1 | AAGCAAA | ALQ | NLR | HLR | SLT | ELV | NLV | | | ical |
| L1 | | | ATT (SEQ | N | A | E | E | R | R | | | |
| | | | ID NO: | (SEQ | (SEQ | (SEQ | (SEQ | (SEQ | (SEQ | | | |
| | | | 917) | ID | ID | ID | ID | ID | ID | | | |
| | | | | NO: | NO: | NO: | NO: | NO: | NO: | | | |
| | | | | 768) | 753) | 762) | 773) | 792) | 787) | | | |
| 3xG32- | Human | TRA | GGAGAAG | TSG | QSS | QRA | — | — | — | RB | DddA^{N} | N- |
| TRAM1 | | M1L1 | AT | NLV | NLV | HLE | | | | | | terminal |
| L1 | | | | R | R | R | | | | | | |
| | | | | (SEQ | (SEQ | (SEQ | | | | | | |
| | | | | ID | ID | ID | | | | | | |
| | | | | NO: | NO: | NO: | | | | | | |
| | | | | 788) | 785) | 793) | | | | | | |
| 4xG32- | Human | TRA | GCAGGAG | TSG | QSS | QRA | QSG | — | — | RB | DddA^{N} | N- |
| TRAM1 | | M1L1 | AAGAT | NLV | NLV | HLE | DLR | | | | | terminal |
| L1 | | | (SEQ ID | R | R | R | R | | | | | |
| | | | NO: 900) | (SEQ | (SEQ | (SEQ | (SEQ | | | | | |
| | | | | ID | ID | ID | ID | | | | | |
| | | | | NO: | NO: | NO: | NO: | | | | | |
| | | | | 788) | 785) | 793) | 789) | | | | | |
| 5xG32- | Human | TRA | AGGGCAG | TSG | QSS | QRA | QSG | RSD | — | RB | DddA^{N} | N- |
| TRAM1 | | M1L1 | GAGAAGA | NLV | NLV | HLE | DLR | HLT | | | | terminal |
| L1 | | | T (SEQ ID | R | R | R | R | N | | | | |
| | | | NO: 909) | (SEQ | (SEQ | (SEQ | (SEQ | (SEQ | | | | |
| | | | | ID | ID | ID | ID | ID | | | | |
| | | | | NO: | NO: | NO: | NO: | NO: | | | | |
| | | | | 788) | 785) | 793) | 789) | 763) | | | | |
| 6xG32- | Human | TRA | GGAAGGG | TSG | QSS | QRA | QSG | RSD | QRA | RB | DddA^{N} | N- |
| TRAM1 | | M1L1 | CAGGAGA | NLV | NLV | HLE | DLR | HLT | HLE | | | terminal |
| L1 | | | AGAT (SEQ | R | R | R | R | N | R | | | |
| | | | ID NO: | (SEQ | (SEQ | (SEQ | (SEQ | (SEQ | (SEQ | | | |
| | | | 918) | ID | ID | ID | ID | ID | ID | | | |
| | | | | NO: | NO: | NO: | NO: | NO: | NO: | | | |
| | | | | 788) | 785) | 793) | 789) | 763) | 793) | | | |
| LT30- | Human | HBB | CTGGGCAT | QKS | DPG | RND | — | — | — | LT | DddA^{N} | N- |
| HBB | | | A | SLIA | HLV | ALT | | | | | or | terminal |
| | | | | (SEQ | R | E | | | | | DddA^{C} | |
| | | | | ID | (SEQ | (SEQ | | | | | | |
| | | | | NO: | ID | ID | | | | | | |
| | | | | 765) | NO: | NO: | | | | | | |
| | | | | | 794) | 783) | | | | | | |
| LT31- | Human | HBB | GCTGGGC | TSG | RSD | TSG | — | — | — | LT | DddA^{N} | N- |
| HBB | | | AT | NLT | KLV | ELV | | | | | or | terminal |
| | | | | E | R | R | | | | | DddA^{C} | |
| | | | | (SEQ | (SEQ | (SEQ | | | | | | |
| | | | | ID | ID | ID | | | | | | |
| | | | | NO: | NO: | NO: | | | | | | |
| | | | | 772) | 795) | 792) | | | | | | |
| LT32- | Human | HBB | GGCTGGG | QSG | RSD | DPG | — | — | — | LT | DddA^{N} | N- |
| HBB | | | CA | DLE | HLT | HLV | | | | | or | terminal |
| | | | | R | T | R | | | | | DddA^{C} | |
| | | | | (SEQ | (SEQ | (SEQ | | | | | | |
| | | | | ID | ID | ID | | | | | | |
| | | | | NO: | NO: | NO: | | | | | | |
| | | | | 789) | 811) | 794) | | | | | | |
| LT33- | Human | HBB | GGGCTGG | DPG | RND | RSD | — | — | — | LT | DddA^{N} | N- |
| HBB | | | GC | HLV | ALT | KLV | | | | | or | terminal |
| | | | | R | E | R | | | | | DddA^{C} | |
| | | | | (SEQ | (SEQ | (SEQ | | | | | | |
| | | | | ID | ID | ID | | | | | | |
| | | | | NO: | NO: | NO: | | | | | | |
| | | | | 794) | 783) | 795) | | | | | | |
| LT34- | Human | HBB | AGGGCTG | RSD | TSG | RSD | — | — | — | LT | DddA^{N} | N- |
| HBB | | | GG | KLV | ELV | HLT | | | | | or | terminal |
| | | | | R | R | N | | | | | DddA^{C} | |
| | | | | (SEQ | (SEQ | (SEQ | | | | | | |
| | | | | ID | ID | ID | | | | | | |
| | | | | NO: | NO: | NO: | | | | | | |
| | | | | 795) | 792) | 763) | | | | | | |
| LT35- | Human | HBB | CAGGGCT | RSD | DPG | RAD | — | — | — | LT | DddA^{N} | N- |
| HBB | | | GG | HLT | HLV | NLT | | | | | or | terminal |
| | | | | T | R | E | | | | | DddA^{C} | |
| | | | | (SEQ | (SEQ | (SEQ | | | | | | |
| | | | | ID | ID | ID | | | | | | |
| | | | | NO: | NO: | NO: | | | | | | |
| | | | | 811) | 794) | 771) | | | | | | |
| LT36- | Human | HBB | CCAGGGCT | RND | RSD | TSH | — | — | — | LT | DddA^{N} | N- |
| HBB | | | G | ALT | KLV | SLTE | | | | | or | terminal |
| | | | | E | R | (SEQ | | | | | DddA^{C} | |
| | | | | (SEQ | (SEQ | ID | | | | | | |
| | | | | ID | ID | NO: | | | | | | |
| | | | | NO: | NO: | 773) | | | | | | |
| | | | | 783) | 795) | | | | | | | |
| LT37- | Human | HBB | GCCAGGG | TSG | RSD | DCR | — | — | — | LT | DddA^{N} | N- |
| HBB | | | CT | ELV | HLT | DLA | | | | | or | terminal |
| | | | | R | N | R | | | | | DddA^{C} | |
| | | | | (SEQ | (SEQ | (SEQ | | | | | | |
| | | | | ID | ID | ID | | | | | | |
| | | | | NO: | NO: | NO: | | | | | | |
| | | | | 792) | 763) | 790) | | | | | | |
| LT38- | Human | HBB | AGCCAGG | DPG | RAD | ERS | — | — | — | LT | DddA^{N} | N- |
| HBB | | | GC | HLV | NLT | HLR | | | | | or | terminal |
| | | | | R | E | E | | | | | DddA^{C} | |
| | | | | (SEQ | (SEQ | (SEQ | | | | | | |
| | | | | ID | ID | ID | | | | | | |
| | | | | NO: | NO: | NO: | | | | | | |
| | | | | 794) | 771) | 762) | | | | | | |
| LT39- | Human | HBB | GAGCCAG | RSD | TSH | RSD | — | — | — | LT | DddA^{N} | N- |
| HBB | | | GG | KLV | SLTE | NLV | | | | | or | terminal |
| | | | | R | (SEQ | R | | | | | DddA^{C} | |
| | | | | (SEQ | ID | (SEQ | | | | | | |
| | | | | ID | NO: | ID | | | | | | |
| | | | | NO: | 773) | NO: | | | | | | |
| | | | | 795) | | 787) | | | | | | |
| LT310- | Human | HBB | GGAGCCA | RSD | DCR | QRA | — | — | — | LT | DddA^{N} | N- |
| HBB | | | GG | HLT | DLA | HLE | | | | | or | terminal |
| | | | | N | R | R | | | | | DddA^{C} | |
| | | | | (SEQ | (SEQ | (SEQ | | | | | | |
| | | | | ID | ID | ID | | | | | | |
| | | | | NO: | NO: | NO: | | | | | | |
| | | | | 763) | 790) | 793) | | | | | | |
| LT311- | Human | HBB | AGGAGCC | RAD | ERS | RSD | — | — | — | LT | DddA^{N} | N- |
| HBB | | | AG | NLT | HLR | HLT | | | | | or | terminal |
| | | | | E | E | N | | | | | DddA^{C} | |
| | | | | (SEQ | (SEQ | (SEQ | | | | | | |
| | | | | ID | ID | ID | | | | | | |
| | | | | NO: | NO: | NO: | | | | | | |
| | | | | 771) | 762) | 763) | | | | | | |
| LB30- | Human | HBB | TATGCCCA | RAD | DCR | ARG | — | — | — | LB | DddA^{N} | Canon- |
| HBB | | | G | NLT | DLA | NLR | | | | | or | ical |
| | | | | E | R | T | | | | | DddA^{C} | |
| | | | | (SEQ | (SEQ | (SEQ | | | | | | |
| | | | | ID | ID | ID | | | | | | |
| | | | | NO: | NO: | NO: | | | | | | |
| | | | | 771) | 790) | 804) | | | | | | |
| LB31- | Human | HBB | ATGCCCAG | ERS | SKK | RRD | — | — | — | LB | DddA^{N} | Canon- |
| HBB | | | C | HLR | HLA | ELN | | | | | or | ical |
| | | | | E | E | V | | | | | DddA^{C} | |
| | | | | (SEQ | (SEQ | (SEQ | | | | | | |
| | | | | ID | ID | ID | | | | | | |
| | | | | NO: | NO: | NO: | | | | | | |
| | | | | 762) | 774) | 767) | | | | | | |
| LB32- | Human | HBB | TGCCCAGC | DCR | TSH | APK | — | — | — | LB | DddA^{N} | Canon- |
| HBB | | | C | DLA | SLTE | ALG | | | | | or | ical |
| | | | | R | (SEQ | W | | | | | DddA^{C} | |
| | | | | (SEQ | ID | (SEQ | | | | | | |
| | | | | ID | NO: | ID | | | | | | |
| | | | | NO: | 773) | NO: | | | | | | |
| | | | | 790) | | 810) | | | | | | |
| LB33- | Human | HBB | GCCCAGCC | SKK | RAD | DCR | — | — | — | LB | DddA^{N} | Canon- |
| HBB | | | C | HLA | NLT | DLA | | | | | or | ical |
| | | | | E | E | R | | | | | DddA^{C} | |
| | | | | (SEQ | (SEQ | (SEQ | | | | | | |
| | | | | ID | ID | ID | | | | | | |
| | | | | NO: | NO: | NO: | | | | | | |
| | | | | 774) | 771) | 790) | | | | | | |
| LB34- | Human | HBB | CCCAGCCC | TKN | ERS | SKK | — | — | — | LB | DddA^{N} | Canon- |
| HBB | | | T | SLTE | HLR | HLA | | | | | or | ical |
| | | | | (SEQ | E | E | | | | | DddA^{C} | |
| | | | | ID | (SEQ | (SEQ | | | | | | |
| | | | | NO: | ID | ID | | | | | | |
| | | | | 776) | NO: | NO: | | | | | | |
| | | | | | 762) | 774) | | | | | | |
| LB35- | Human | HBB | CCAGCCCT | RND | DCR | TSH | — | — | — | LB | DddA^{N} | Canon- |
| HBB | | | G | ALT | DLA | SLTE | | | | | or | ical |
| | | | | E | R | (SEQ | | | | | DddA^{C} | |
| | | | | (SEQ | (SEQ | ID | | | | | | |
| | | | | ID | ID | NO: | | | | | | |
| | | | | NO: | NO: | 773) | | | | | | |
| | | | | 783) | 790) | | | | | | | |
| LB36- | Human | HBB | CAGCCCTG | RSD | SKK | RAD | — | — | — | LB | DddA^{N} | Canon- |
| HBB | | | G | HLT | HLA | NLT | | | | | or | ical |
| | | | | T | E | E | | | | | DddA^{C} | |
| | | | | (SEQ | (SEQ | (SEQ | | | | | | |
| | | | | ID | ID | ID | | | | | | |
| | | | | NO: | NO: | NO: | | | | | | |
| | | | | 811) | 774) | 771) | | | | | | |
| LB37- | Human | HBB | AGCCCTGG | DPG | TKN | ERS | — | — | — | LB | DddA^{N} | Canon- |
| HBB | | | C | HLV | SLTE | HLR | | | | | or | ical |
| | | | | R | (SEQ | E | | | | | DddA^{C} | |
| | | | | (SEQ | ID | (SEQ | | | | | | |
| | | | | ID | NO: | ID | | | | | | |
| | | | | NO: | 776) | NO: | | | | | | |
| | | | | 794) | | 762) | | | | | | |
| LB38- | Human | HBB | GCCCTGGC | TSG | RND | DCR | — | — | — | LB | DddA^{N} | Canon- |
| HBB | | | T | ELV | ALT | DLA | | | | | or | ical |
| | | | | R | E | R | | | | | DddA^{C} | |
| | | | | (SEQ | (SEQ | (SEQ | | | | | | |
| | | | | ID | ID | ID | | | | | | |
| | | | | NO: | NO: | NO: | | | | | | |
| | | | | 792) | 783) | 790) | | | | | | |
| LB39- | Human | HBB | CCCTGGCT | QRH | RSD | SKK | — | — | — | LB | DddA^{N} | Canon- |
| HBB | | | C | HLV | HLT | HLA | | | | | or | ical |
| | | | | E | T | E | | | | | DddA^{C} | |
| | | | | (SEQ | (SEQ | (SEQ | | | | | | |
| | | | | ID | ID | ID | | | | | | |
| | | | | NO: | NO: | NO: | | | | | | |
| | | | | 782) | 811) | 774) | | | | | | |
| LB310- | Human | HBB | CCTGGCTC | RSD | DPG | TKN | — | — | — | LB | DddA^{N} | Canon- |
| HBB | | | C | ERK | HLV | SLTE | | | | | or | ical |
| | | | | R | R | (SEQ | | | | | DddA^{C} | |
| | | | | (SEQ | (SEQ | ID | | | | | | |
| | | | | ID | ID | NO: | | | | | | |
| | | | | NO: | NO: | 776) | | | | | | |
| | | | | 806) | 794) | | | | | | | |
| LB311- | Human | HBB | CTGGCTCC | TKN | TSG | RND | — | — | — | LB | DddA^{N} | Canon- |
| HBB | | | T | SLTE | ELV | ALT | | | | | or | ical |
| | | | | (SEQ | R | E | | | | | DddA^{C} | |
| | | | | ID | (SEQ | (SEQ | | | | | | |
| | | | | NO: | ID | ID | | | | | | |
| | | | | 776) | NO: | NO: | | | | | | |
| | | | | | 792) | 783) | | | | | | |
| RT30- | Human | HBB | AAGTCAG | RSD | RSD | RKD | — | — | — | RT | DddA^{N} | Canon- |
| HBB | | | GG | KLV | HLT | NLK | | | | | or | ical |
| | | | | R | T | N | | | | | DddA^{C} | |
| | | | | (SEQ | (SEQ | (SEQ | | | | | | |
| | | | | ID | ID | ID | | | | | | |
| | | | | NO: | NO: | NO: | | | | | | |
| | | | | 795) | 811) | 755) | | | | | | |
| RT31- | Human | HBB | AGTCAGG | DPG | RAD | HRT | — | — | — | RT | DddA^{N} | Canon- |
| HBB | | | GC | HLV | NLT | TLT | | | | | or | ical |
| | | | | R | E | N | | | | | DddA^{C} | |
| | | | | (SEQ | (SEQ | (SEQ | | | | | | |
| | | | | ID | ID | ID | | | | | | |
| | | | | NO: | NO: | NO: | | | | | | |
| | | | | 794) | 771) | 764) | | | | | | |
| RT32- | Human | HBB | GTCAGGG | QSG | RSD | DPG | — | — | — | RT | DddA^{N} | Canon- |
| HBB | | | CA | DLR | HLT | ALV | | | | | or | ical |
| | | | | R | N | R | | | | | DddA^{C} | |
| | | | | (SEQ | (SEQ | (SEQ | | | | | | |
| | | | | ID | ID | ID | | | | | | |
| | | | | NO: | NO: | NO: | | | | | | |
| | | | | 789) | 763) | 798) | | | | | | |
| RT33- | Human | HBB | TCAGGGC | RAD | RSD | RSD | — | — | — | RT | DddA^{N} | Canon- |
| HBB | | | AG | NLT | KLV | HLT | | | | | or | ical |
| | | | | E | R | T | | | | | DddA^{C} | |
| | | | | (SEQ | (SEQ | (SEQ | | | | | | |
| | | | | ID | ID | ID | | | | | | |
| | | | | NO: | NO: | NO: | | | | | | |
| | | | | 771) | 795) | 811) | | | | | | |
| RT34- | Human | HBB | CAGGGCA | QLA | DPG | RAD | — | — | — | RT | DddA^{N} | Canon- |
| HBB | | | GA | HLR | HLV | NLT | | | | | or | ical |
| | | | | A | R | E | | | | | DddA^{C} | |
| | | | | (SEQ | (SEQ | (SEQ | | | | | | |
| | | | | ID | ID | ID | | | | | | |
| | | | | NO: | NO: | NO: | | | | | | |
| | | | | 761) | 794) | 771) | | | | | | |
| RT35- | Human | HBB | AGGGCAG | RSD | QSG | RSD | — | — | — | RT | DddA^{N} | Canon- |
| HBB | | | AG | NLV | DLR | HLT | | | | | or | ical |
| | | | | R | R | N | | | | | DddA^{C} | |
| | | | | (SEQ | (SEQ | (SEQ | | | | | | |
| | | | | ID | ID | ID | | | | | | |
| | | | | NO: | NO: | NO: | | | | | | |
| | | | | 787) | 789) | 763) | | | | | | |
| RT36- | Human | HBB | GGGCAGA | ERS | RAD | RSD | — | — | — | RT | DddA^{N} | Canon- |
| HBB | | | GC | HLR | NLT | KLV | | | | | or | ical |
| | | | | E | E | R | | | | | DddA^{C} | |
| | | | | (SEQ | (SEQ | (SEQ | | | | | | |
| | | | | ID | ID | ID | | | | | | |
| | | | | NO: | NO: | NO: | | | | | | |
| | | | | 762) | 771) | 795) | | | | | | |
| RT37- | Human | HBB | GGCAGAG | DCR | QLA | DPG | — | — | — | RT | DddA^{N} | Canon- |
| HBB | | | CC | DLA | HLR | HLV | | | | | or | ical |
| | | | | R | A | R | | | | | DddA^{C} | |
| | | | | (SEQ | (SEQ | (SEQ | | | | | | |
| | | | | ID | ID | ID | | | | | | |
| | | | | NO: | NO: | NO: | | | | | | |
| | | | | 790) | 761) | 794) | | | | | | |
| RT38- | Human | HBB | GCAGAGC | TSH | RSD | QSG | — | — | — | RT | DddA^{N} | Canon- |
| HBB | | | CA | SLTE | NLV | DLR | | | | | or | ical |
| | | | | (SEQ | R | R | | | | | DddA^{C} | |
| | | | | ID | (SEQ | (SEQ | | | | | | |
| | | | | NO: | ID | ID | | | | | | |
| | | | | 773) | NO: | NO: | | | | | | |
| | | | | | 787) | 789) | | | | | | |
| RT39- | Human | HBB | CAGAGCC | TSG | ERS | RAD | — | — | — | RT | DddA^{N} | Canon- |
| HBB | | | AT | NLT | HLR | NLT | | | | | or | ical |
| | | | | E | E | E | | | | | DddA^{C} | |
| | | | | (SEQ | (SEQ | (SEQ | | | | | | |
| | | | | ID | ID | ID | | | | | | |
| | | | | NO: | NO: | NO: | | | | | | |
| | | | | 772) | 762) | 771) | | | | | | |
| RT310- | Human | HBB | AGAGCCA | RRS | DCR | QLA | — | — | — | RT | DddA^{N} | Canon- |
| HBB | | | TC | ACR | DLA | HLR | | | | | or | ical |
| | | | | R | R | A | | | | | DddA^{C} | |
| | | | | (SEQ | (SEQ | (SEQ | | | | | | |
| | | | | ID | ID | ID | | | | | | |
| | | | | NO: | NO: | NO: | | | | | | |
| | | | | 766) | 790) | 761) | | | | | | |
| RT311- | Human | HBB | GAGCCATC | RLR | TSH | RSD | — | — | — | RT | DddA^{N} | Canon- |
| HBB | | | T | DIQF | SLTE | NLV | | | | | or | ical |
| | | | | (SEQ | (SEQ | R | | | | | DddA^{C} | |
| | | | | ID | ID | (SEQ | | | | | | |
| | | | | NO: | NO: | ID | | | | | | |
| | | | | 808) | 773) | NO: | | | | | | |
| | | | | | | 787) | | | | | | |
| RB30- | Human | HBB | CCCTGACT | TTG | QAG | SKK | — | — | — | RB | DddA^{N} | N- |
| HBB | | | T | ALT | HLA | HLA | | | | | or | terminal |
| | | | | E | S | E | | | | | DddA^{C} | |
| | | | | (SEQ | (SEQ | (SEQ | | | | | | |
| | | | | ID | ID | ID | | | | | | |
| | | | | NO: | NO: | NO: | | | | | | |
| | | | | 784) | 809) | 774) | | | | | | |
| RB31- | Human | HBB | GCCCTGAC | THL | RND | DCR | — | — | — | RB | DddA^{N} | N- |
| HBB | | | T | DLIR | ALT | DLA | | | | | or | terminal |
| | | | | (SEQ | E | R | | | | | DddA^{C} | |
| | | | | ID | (SEQ | (SEQ | | | | | | |
| | | | | NO: | ID | ID | | | | | | |
| | | | | 760) | NO: | NO: | | | | | | |
| | | | | | 783) | 790) | | | | | | |
| RB32- | Human | HBB | TGCCCTGA | DPG | TKN | APK | — | — | — | RB | DddA^{N} | N- |
| HBB | | | C | NLV | SLTE | ALG | | | | | or | terminal |
| | | | | R | (SEQ | W | | | | | DddA^{C} | |
| | | | | (SEQ | ID | (SEQ | | | | | | |
| | | | | ID | NO: | ID | | | | | | |
| | | | | NO: | 776) | NO: | | | | | | |
| | | | | 786) | | 810) | | | | | | |
| RB33- | Human | HBB | CTGCCCTG | QAG | SKK | RND | — | — | — | RB | DddA^{N} | N- |
| HBB | | | A | HLA | HLA | ALT | | | | | or | terminal |
| | | | | S | E | E | | | | | DddA^{C} | |
| | | | | (SEQ | (SEQ | (SEQ | | | | | | |
| | | | | ID | ID | ID | | | | | | |
| | | | | NO: | NO: | NO: | | | | | | |
| | | | | 809) | 774) | 783) | | | | | | |
| RB34- | Human | HBB | TCTGCCCT | RND | DCR | RLR | — | — | — | RB | DddA^{N} | N- |
| HBB | | | G | ALT | DLA | DIQF | | | | | or | terminal |
| | | | | E | R | (SEQ | | | | | DddA^{C} | |
| | | | | (SEQ | (SEQ | ID | | | | | | |
| | | | | ID | ID | NO: | | | | | | |
| | | | | NO: | NO: | 808) | | | | | | |
| | | | | 783) | 790) | | | | | | | |
| RB35- | Human | HBB | CTCTGCCC | TKN | APK | QRH | — | — | — | RB | DddA^{N} | N- |
| HBB | | | T | SLTE | ALG | HLV | | | | | or | terminal |
| | | | | (SEQ | W | E | | | | | DddA^{C} | |
| | | | | ID | (SEQ | (SEQ | | | | | | |
| | | | | NO: | ID | ID | | | | | | |
| | | | | 776) | NO: | NO: | | | | | | |
| | | | | | 810) | 782) | | | | | | |
| RB36- | Human | HBB | GCTCTGCC | SKK | RND | TSG | — | — | — | RB | DddA^{N} | N- |
| HBB | | | C | HLA | ALT | ELV | | | | | or | terminal |
| | | | | E | E | R | | | | | | |
| | | | | (SEQ | (SEQ | (SEQ | | | | | DddA^{C} | |
| | | | | ID | ID | ID | | | | | | |
| | | | | NO: | NO: | NO: | | | | | | |
| | | | | 774) | 783) | 792) | | | | | | |
| RB37- | Human | HBB | GGCTCTGC | DCR | RLR | DPG | — | — | — | RB | DddA^{N} | N- |
| HBB | | | C | DLA | DIQF | HLV | | | | | or | terminal |
| | | | | R | (SEQ | R | | | | | DddA^{C} | |
| | | | | (SEQ | ID | (SEQ | | | | | | |
| | | | | ID | NO: | ID | | | | | | |
| | | | | NO: | 808) | NO: | | | | | | |
| | | | | 790) | | 794) | | | | | | |
| RB38- | Human | HBB | TGGCTCTG | APK | QRH | RSD | — | — | — | RB | DddA^{N} | N- |
| HBB | | | C | ALG | HLV | HLT | | | | | or | terminal |
| | | | | W | E | T | | | | | DddA^{C} | |
| | | | | (SEQ | (SEQ | (SEQ | | | | | | |
| | | | | ID | ID | ID | | | | | | |
| | | | | NO: | NO: | NO: | | | | | | |
| | | | | 810) | 782) | 811) | | | | | | |
| RB39- | Human | HBB | ATGGCTCT | RND | TSG | RRD | — | — | — | RB | DddA^{N} | N- |
| HBB | | | G | ALT | ELV | ELN | | | | | or | terminal |
| | | | | E | R | V | | | | | DddA^{C} | |
| | | | | (SEQ | (SEQ | (SEQ | | | | | | |
| | | | | ID | ID | ID | | | | | | |
| | | | | NO: | NO: | NO: | | | | | | |
| | | | | 783) | 792) | 767) | | | | | | |
| RB310- | Human | HBB | GATGGCTC | RLR | DPG | TSG | — | — | — | RB | DddA^{N} | N- |
| HBB | | | T | DIQF | HLV | NLV | | | | | or | terminal |
| | | | | (SEQ | R | R | | | | | DddA^{C} | |
| | | | | ID | (SEQ | (SEQ | | | | | | |
| | | | | NO: | ID | ID | | | | | | |
| | | | | 808) | NO: | NO: | | | | | | |
| | | | | | 794) | 788) | | | | | | |
| RB311- | Human | HBB | AGATGGCT | QRH | RSD | QLA | — | — | — | RB | DddA^{N} | N- |
| HBB | | | C | HLV | HLT | HLR | | | | | or | terminal |
| | | | | E | T | A | | | | | DddA^{C} | |
| | | | | (SEQ | (SEQ | (SEQ | | | | | | |
| | | | | ID | ID | ID | | | | | | |
| | | | | NO: | NO: | NO: | | | | | | |
| | | | | 782) | 811) | 761) | | | | | | |
| RB610- | V | HBB | TAAGCAAT | RLR | DPG | TSG | QKS | QSG | QAS | RB | DddA^{C} | N- |
| HBB | an | | AGATGGCT | DIQF | HLV | NLV | SLIA | DLR | NLIS | | | terminal |
| | | | CT (SEQ ID | (SEQ | R | R | (SEQ | R | (SEQ | | | |
| | | | NO: 919) | ID | (SEQ | (SEQ | ID | (SEQ | ID | | | |
| | | | | NO: | ID | ID | NO: | ID | NO: | | | |
| | | | | 808) | NO: | NO: | 765) | NO: | 801) | | | |
| | | | | | 794) | 788) | | 789) | | | | |

TABLE 9
ZF Codons.
The following amino acid sequences are inserted into each ZF
repeat in between the beta-motif and alpha-motif,
according to the target DNA sequence.

| Target | | | SEQ ID NO |
|---|---|---|---|
| DNA | | | for ZF |
| sequence | ZF amino acid | ZF nucleotide | nucleotide |
| (5′ to 3′) | sequence | sequence | sequence: |
| AAA | QRANLRA (SEQ | cagagagctaatctcag | 816 |
| | ID NO: 753) | ggcc | |
| AAC | DSGNLRV (SEQ | gattcagggaatctccg | 817 |
| | ID NO: 754) | ggtt | |
| AAG | RKDNLKN (SEQ | cgaaaagataatctgaa | 818 |
| | ID NO: 755) | gaat | |
| AAT | TTGNLTV (SEQ | accactggaaacctcac | 819 |
| | ID NO: 756) | ggtg | |
| ACA | SPADLTR (SEQ | agtcctgcagatcttacc | 820 |
| | ID NO: 757) | cga | |
| ACC | DKKDLTR (SEQ | gacaagaaggatctgac | 821 |
| | ID NO: 758) | acga | |
| ACG | RTDTLRD (SEQ | aggactgatacgctgcg | 822 |
| | ID NO: 759) | cgat | |
| ACT | THLDLIR (SEQ | acccacctggacctcat | 823 |
| | ID NO: 760) | caga | |
| AGA | QLAHLRA (SEQ | caactcgctcatctgcga | 824 |
| | ID NO: 761) | gca | |
| AGC | ERSHLRE (SEQ | gaacgaagccacctgc | 825 |
| | ID NO: 762) | gcgaa | |
| AGG | RSDHLTN (SEQ | cgcagcgaccatttgac | 826 |
| | ID NO: 763) | taac | |
| AGT | HRTTLTN (SEQ | caccgaacgaccttgac | 827 |
| | ID NO: 764) | taac | |
| ATA | QKSSLIA (SEQ | cagaaatcttctttgatag | 828 |
| | ID NO: 765) | ct | |
| ATC | RRSACRR (SEQ | cggagatcagcctgtcg | 829 |
| | ID NO: 766) | acgc | |
| ATG | RRDELNV (SEQ | aggcgggacgaactga | 830 |
| | ID NO: 767) | acgtg | |
| ATT | HKNALQN (SEQ | cacaaaaatgccttgca | 831 |
| | ID NO: 768) | aaac | |
| CAA | QSGNLTE (SEQ | caatctggcaatcttaca | 832 |
| | ID NO: 769) | gag | |
| CAC | SKKALTE (SEQ | tctaaaaaggcgctgac | 833 |
| | ID NO: 770) | ggag | |
| CAG | RADNLTE (SEQ | cgggcggataatctcac | 834 |
| | ID NO: 771) | tgag | |
| CAT | TSGNLTE (SEQ | acgagtggaaatcttac | 835 |
| | ID NO: 772) | ggaa | |
| CCA | TSHSLTE (SEQ | acgtcccacagtttgacc | 836 |
| | ID NO: 773) | gaa | |
| CCC | SKKHLAE (SEQ | agcaagaaacaccttgc | 837 |
| | ID NO: 774) | agaa | |
| CCG | RNDTLTE (SEQ | aggaatgatactcttacc | 838 |
| | ID NO: 775) | gag | |
| CCT | TKNSLTE (SEQ | acaaagaacagcctcac | 839 |
| | ID NO: 776) | cgag | |
| CGA | QSGHLTE (SEQ | cagtcagggcatctcac | 840 |
| | ID NO: 777) | ggag | |
| CGC | HTGHLLE (SEQ | cacacaggccatttgttg | 841 |
| | ID NO: 778) | gag | |
| CGG | RSDKLTE (SEQ | cggagtgataaactcac | 842 |
| | ID NO: 779) | cgaa | |
| CGT | SRRTCRA (SEQ | tcacgacgcacctgtag | 843 |
| | ID NO: 780) | agcg | |
| CTA | QNSTLTE (SEQ | cagaattcaactctcacc | 844 |
| | ID NO: 781) | gaa | |
| CTC | QRHHLVE (SEQ | cagcgacaccatttggt | 845 |
| | ID NO: 782) | cgag | |
| CTG | RNDALTE (SEQ | cggaacgatgcacttac | 846 |
| | ID NO: 783) | cgag | |
| CTT | TTGALTE (SEQ | actacaggggctctcac | 847 |
| | ID NO: 784) | tgaa | |
| GAA | QSSNLVR (SEQ | cagagtagtaacctggt | 848 |
| | ID NO: 785) | gagg | |
| GAC | DPGNLVR (SEQ | gatcccgggaacctcgt | 849 |
| | ID NO: 786) | taga | |
| GAG | RSDNLVR (SEQ | cgctctgataacctggtc | 850 |
| | ID NO: 787) | aga | |
| GAT | TSGNLVR (SEQ | actagcgggaacctcgt | 851 |
| | ID NO: 788) | ccgg | |
| GCA | QSGDLRR (SEQ | caaagcggggacttga | 852 |
| | ID NO: 789) | gaagg | |
| GCC | DCRDLAR (SEQ | gattgccgagatcttgct | 853 |
| | ID NO: 790) | cgg | |
| GCG | RSDDLVR (SEQ | cgctcagatgatctggtt | 854 |
| | ID NO: 791) | cgc | |
| GCT | TSGELVR (SEQ | acgtctggggagttggtt | 855 |
| | ID NO: 792) | agg | |
| GGA | QRAHLER (SEQ | caaagagcccatctgga | 856 |
| | ID NO: 793) | aagg | |
| GGC | DPGHLVR (SEQ | gatcccggacacttggtt | 857 |
| | ID NO: 794) | cga | |
| GGG | RSDKLVR (SEQ | cgcagcgacaaactcgt | 858 |
| | ID NO: 795) | taga | |
| GGT | TSGHLVR (SEQ | acttcaggccatcttgta | 859 |
| | ID NO: 796) | aga | |
| GTA | QSSSLVR (SEQ | caatcttcctcacttgtga | 860 |
| | ID NO: 797) | gg | |
| GTC | DPGALVR (SEQ | gacccaggggctttggt | 861 |
| | ID NO: 798) | tcgg | |
| GTG | RSDELVR (SEQ | cggtcagatgagctggt | 862 |
| | ID NO: 799) | acgc | |
| GTT | TSGSLVR (SEQ | acaagcggctctctcgtt | 863 |
| | ID NO: 800) | aga | |
| TAA | QASNLIS (SEQ | caagcctctaacttgatt | 864 |
| | ID NO: 801) | agc | |
| TAC | SRGNLKS (SEQ | agcaggggtaacttgaa | 865 |
| | ID NO: 802) | atcc | |
| TAG | REDNLHT (SEQ | cgggaagacaaccttca | 866 |
| | ID NO: 803) | tacg | |
| TAT | ARGNLRT (SEQ | gcacgcgggaacttgc | 867 |
| | ID NO: 804) | ggact | |
| TCA | RSDHLTT (SEQ | cgaagtgatcacttgac | 868 |
| | ID NO: 811) | aacc | |
| TCC | RSDERKR (SEQ | cggtcagacgagagaa | 869 |
| | ID NO: 806) | agcga | |
| TCG | RLRALDR (SEQ | cgcttgcgggcgctcga | 870 |
| | ID NO: 807) | ccga | |
| TCT | RLRDIQF (SEQ | agactcagggatataca | 871 |
| | ID NO: 808) | attt | |
| TGA | QAGHLAS (SEQ | caagcgggccacctcg | 872 |
| | ID NO: 809) | ccagc | |
| TGC | APKALGW (SEQ | gccccaaaagcactgg | 873 |
| | ID NO: 810) | gctgg | |
| TGG | RSDHLTT (SEQ | cggagcgaccatctcac | 874 |
| | ID NO: 811) | tact | |
| TGT | WRDSLLA (SEQ | tggcgcgactcccttctc | 875 |
| | ID NO: 812) | gcg | |
| TTA | QKWPRDS (SEQ | cagaagtggcccaggg | 876 |
| | ID NO: 813) | attca | |
| TTC | DNSYLPR (SEQ | gacaattcttacttgccc | 877 |
| | ID NO: 814) | agg | |
| TTG | RKDALRG (SEQ | aggaaagatgcgcttag | 878 |
| | ID NO: 815) | aggg | |
| TTT | — | — | |

```
TABLE 10
Optimized ZF scaffolds
For canonical ZF scaffolds see FIG. S6a-d.
All ZF scaffolds contain an N-terminal
    cap MAERP and a C-terminal cap
  HTKIHLR unless otherwise specified.
```

| ZF | | Alpha- | Linker |
|---|---|---|---|
| scaffold | Beta-motif | motif | motif |
| X1 | FACDICGRKFA | HIRTH | TGEKP |
| X2 | FACDICGRKFA | HIRTH | TGQKP |
| X3 | FACDICGRKFA | HTKIH | TGEKP |
| X4 | FACDICGRKFA | HTKIH | TGQKP |
| X5 | FQCRICMRNFS | HIRTH | TGEKP |
| X6 | FQCRICMRNFS | HIRTH | TGQKP |
| X7 | FQCRICMRNFS | HTKIH | TGEKP |
| X8 | FQCRICMRNFS | HTKIH | TGQKP |
| KGKS | YKCPECGKSFS | HIRTH | TGEKP |
| AGKS | YACPECGKSFS | HIRTH | TGEKP |
| AGRS | YACPECGRSFS | HIRTH | TGEKP |
| ADRS | YACPECDRSFS | HIRTH | TGEKP |
| ADRR | YACPECDRRFS | HIRTH | TGEKP |
| VSDRR | YACPVESCDRRFS | HIRTH | TGEKP |
| VSDRS | YACPVESCDRSFS | HIRTH | TGEKP |
| VSGRS | YACPVESCGRSFS | HIRTH | TGEKP |
| VSGRS | YACPVESCGKSFS | HIRTH | TGEKP |
| *V2 | YKCEECGKAFN | HMKIH | TGEKP |
| V20 | YKCEECGKAFN | HMKIH | TGEKP |
| YL1 | FACDICGRKFA | HIRTH | TGEKP |
| YL2 | FACDICGRKFA | HIRTH | TGERP |
| YL3 | FACDICGRKFA | HIRTH | TGKKP |
| YL4 | FACDICGRKFA | HIRTH | TGKRP |
| YL5 | FACDICGRKFA | HIRTH | TGDKP |
| YL6 | FACDICGRKFA | HIRTH | TGDRP |
| YL7 | FACDICGRKFA | HIRTH | TEEKP |
| YL8 | FACDICGRKFA | HIRTH | TEERP |
| YL9 | FACDICGRKFA | HIRTH | TEKKP |
| YL10 | FACDICGRKFA | HIRTH | TEKRP |
| YL11 | FACDICGRKFA | HIRTH | TEDKP |
| YL12 | FACDICGRKFA | HIRTH | TEDRP |
| YL13 | FACDICGRKFA | HIRTH | SGEKP |
| YL14 | FACDICGRKFA | HIRTH | SGERP |
| YL15 | FACDICGRKFA | HIRTH | SGKKP |
| YL16 | FACDICGRKFA | HIRTH | SGKRP |
| YL17 | FACDICGRKFA | HIRTH | SGDKP |
| YL18 | FACDICGRKFA | HIRTH | SGDRP |
| YL19 | FACDICGRKFA | HIRTH | SEEKP |
| YL20 | FACDICGRKFA | HIRTH | SEERP |
| YL21 | FACDICGRKFA | HIRTH | SEKKP |
| YL22 | FACDICGRKFA | HIRTH | SEKRP |
| YL23 | FACDICGRKFA | HIRTH | SEDKP |
| YL24 | FACDICGRKFA | HIRTH | SEDRP |
| YA1 | FACDICGRKFA | HQRIH | TGEKP |
| YA2 | FACDICGRKFA | HQRVH | TGEKP |
| YA3 | FACDICGRKFA | HQRTH | TGEKP |
| YA4 | FACDICGRKFA | HQKIH | TGEKP |
| YA5 | FACDICGRKFA | HQKVH | TGEKP |
| YA6 | FACDICGRKFA | HQKTH | TGEKP |
| YA7 | FACDICGRKFA | HMRIH | TGEKP |
| YA8 | FACDICGRKFA | HMRVH | TGEKP |
| YA9 | FACDICGRKFA | HMRTH | TGEKP |
| YA10 | FACDICGRKFA | HMKIH | TGEKP |
| YA11 | FACDICGRKFA | HMKVH | TGEKP |
| YA12 | FACDICGRKFA | HMKTH | TGEKP |
| YA13 | FACDICGRKFA | HKRIH | TGEKP |
| YA14 | FACDICGRKFA | HKRVH | TGEKP |
| YA15 | FACDICGRKFA | HKRTH | TGEKP |
| YA16 | FACDICGRKFA | HKKIH | TGEKP |
| YA17 | FACDICGRKFA | HKKVH | TGEKP |
| YA18 | FACDICGRKFA | HKKTH | TGEKP |
| YB1 | YKCKECGKAFS | HIRTH | TGEKP |
| YB2 | YKCKECGKAFR | HIRTH | TGEKP |
| YB3 | YKCKECGKAFN | HIRTH | TGEKP |
| YB4 | YKCKECGKSFS | HIRTH | TGEKP |
| YB5 | YKCKECGKSFR | HIRTH | TGEKP |
| YB6 | YKCKECGKSFN | HIRTH | TGEKP |
| YB7 | YKCNECGKAFS | HIRTH | TGEKP |
| YB8 | YKCNECGKAFR | HIRTH | TGEKP |
| YB9 | YKCNECGKAFN | HIRTH | TGEKP |
| YB10 | YKCNECGKSFS | HIRTH | TGEKP |
| YB11 | YKCNECGKSFR | HIRTH | TGEKP |
| YB12 | YKCNECGKSFN | HIRTH | TGEKP |
| YB13 | YKCSECGKAFS | HIRTH | TGEKP |
| YB14 | YKCSECGKAFR | HIRTH | TGEKP |
| YB15 | YKCSECGKAFN | HIRTH | TGEKP |
| YB16 | YKCSECGKSFS | HIRTH | TGEKP |
| YB17 | YKCSECGKSFR | HIRTH | TGEKP |
| YB18 | YKCSECGKSFN | HIRTH | TGEKP |
| YB19 | YKCEECGKAFS | HIRTH | TGEKP |
| YB20 | YKCEECGKAFR | HIRTH | TGEKP |
| YB21 | YKCEECGKAFN | HIRTH | TGEKP |
| YB22 | YKCEECGKSFS | HIRTH | TGEKP |
| YB23 | YKCEECGKSFR | HIRTH | TGEKP |
| YB24 | YKCEECGKSFN | HIRTH | TGEKP |
| YB25 | YECKECGKAFS | HIRTH | TGEKP |
| YB26 | YECKECGKAFR | HIRTH | TGEKP |
| YB27 | YECKECGKAFN | HIRTH | TGEKP |
| YB28 | YECKECGKSFS | HIRTH | TGEKP |
| YB29 | YECKECGKSFR | HIRTH | TGEKP |
| YB30 | YECKECGKSFN | HIRTH | TGEKP |
| YB31 | YECNECGKAFS | HIRTH | TGEKP |
| YB32 | YECNECGKAFR | HIRTH | TGEKP |
| YB33 | YECNECGKAFN | HIRTH | TGEKP |
| YB34 | YECNECGKSFS | HIRTH | TGEKP |
| YB35 | YECNECGKSFR | HIRTH | TGEKP |
| YB36 | YECNECGKSFN | HIRTH | TGEKP |
| YB37 | YECSECGKAFS | HIRTH | TGEKP |
| YB38 | YECSECGKAFR | HIRTH | TGEKP |
| YB39 | YECSECGKAFN | HIRTH | TGEKP |
| YB40 | YECSECGKSFS | HIRTH | TGEKP |
| YB41 | YECSECGKSFR | HIRTH | TGEKP |
| YB42 | YECSECGKSFN | HIRTH | TGEKP |
| YB43 | YECEECGKAFS | HIRTH | TGEKP |
| YB44 | YECEECGKAFR | HIRTH | TGEKP |
| YB45 | YECEECGKAFN | HIRTH | TGEKP |
| YB46 | YECEECGKSFS | HIRTH | TGEKP |
| YB47 | YECEECGKSFR | HIRTH | TGEKP |
| YB48 | YECEECGKSFN | HIRTH | TGEKP |
| YB49 | FKCKECGKAFS | HIRTH | TGEKP |
| YB50 | FKCKECGKAFR | HIRTH | TGEKP |
| YB51 | FKCKECGKAFN | HIRTH | TGEKP |
| YB52 | FKCKECGKSFS | HIRTH | TGEKP |
| YB53 | FKCKECGKSFR | HIRTH | TGEKP |
| YB54 | FKCKECGKSFN | HIRTH | TGEKP |
| YB55 | FKCNECGKAFS | HIRTH | TGEKP |
| YB56 | FKCNECGKAFR | HIRTH | TGEKP |
| YB57 | FKCNECGKAFN | HIRTH | TGEKP |
| YB58 | FKCNECGKSFS | HIRTH | TGEKP |
| YB59 | FKCNECGKSFR | HIRTH | TGEKP |
| YB60 | FKCNECGKSFN | HIRTH | TGEKP |
| YB61 | FKCSECGKAFS | HIRTH | TGEKP |
| YB62 | FKCSECGKAFR | HIRTH | TGEKP |
| YB63 | FKCSECGKAFN | HIRTH | TGEKP |
| YB64 | FKCSECGKSFS | HIRTH | TGEKP |
| YB65 | FKCSECGKSFR | HIRTH | TGEKP |
| YB66 | FKCSECGKSFN | HIRTH | TGEKP |
| YB67 | FKCEECGKAFS | HIRTH | TGEKP |
| YB68 | FKCEECGKAFR | HIRTH | TGEKP |
| YB69 | FKCEECGKAFN | HIRTH | TGEKP |
| YB70 | FKCEECGKSFS | HIRTH | TGEKP |
| YB71 | FKCEECGKSFR | HIRTH | TGEKP |
| YB72 | FKCEECGKSFN | HIRTH | TGEKP |
| YB73 | FECKECGKAFS | HIRTH | TGEKP |
| YB74 | FECKECGKAFR | HIRTH | TGEKP |
| YB75 | FECKECGKAFN | HIRTH | TGEKP |
| YB76 | FECKECGKSFS | HIRTH | TGEKP |
| YB77 | FECKECGKSFR | HIRTH | TGEKP |
| YB78 | FECKECGKSFN | HIRTH | TGEKP |
| YB79 | FECNECGKAFS | HIRTH | TGEKP |
| YB80 | FECNECGKAFR | HIRTH | TGEKP |
| YB81 | FECNECGKAFN | HIRTH | TGEKP |
| YB82 | FECNECGKSFS | HIRTH | TGEKP |
| YB83 | FECNECGKSFR | HIRTH | TGEKP |
| YB84 | FECNECGKSFN | HIRTH | TGEKP |
| YB85 | FECSECGKAFS | HIRTH | TGEKP |
| YB86 | FECSECGKAFR | HIRTH | TGEKP |
| YB87 | FECSECGKAFN | HIRTH | TGEKP |
| YB88 | FECSECGKSFS | HIRTH | TGEKP |
| YB89 | FECSECGKSFR | HIRTH | TGEKP |
| YB90 | FECSECGKSFN | HIRTH | TGEKP |
| YB91 | FECEECGKAFS | HIRTH | TGEKP |
| YB92 | FECEECGKAFR | HIRTH | TGEKP |
| YB93 | FECEECGKAFN | HIRTH | TGEKP |
| YB94 | FECEECGKSFS | HIRTH | TGEKP |
| YB95 | FECEECGKSFR | HIRTH | TGEKP |
| YB96 | FECEECGKSFN | HIRTH | TGEKP |

*ZF scaffold V2 uses a C-terminal cap HMKIHLR

TABLE 11
ZF-DdCBE pairs

| | Optimized ZF scaffold supporting |
|---|---|
| ZF-DdCBE pair | highest on-target editing |
| R8-ATP8 + 4-ATP8 | X1 |
| R8-ATP8 + 10-ATP8 | V2 |
| R8-3i-ATP8 + 4-3i-ATP8 | V2 |
| R8-3i-ATP8 + 10-3ii-ATP8 | V2 |
| 9-ND51 + R13-ND51 | X1 |
| 12-ND51 + R13-ND51 | V20 |
| G24-R1b + G32-R1b | AGKS |
| G22-R13 + G24-R13 | V20 |
| G32-R6a + G21-R6a | AGKS |
| G36-R6c + G212-R6c | AGKS |
| G33-V1 + G35-V1 | V20 |
| G22-V2 + G34-V2 | AGKS |
| G33-V5 + G36-V5 | AGKS |
| ND1-Left + ND1-Right | AGKS |
| ND2-Left + ND2-Right | V20 |
| ND4L-Left + ND4L-Right | X1 |
| ND4-Left + ND4-Right | AGKS |
| ND5-Left + ND5-Right | AGKS |
| ND52-Left + ND52-Right | V20 |
| COX1-Left + COX1-Right | AGKS |
| COX2-Left + COX2-Right | X1 |
| CYB-Left + CYB-Right | V20 |
| G21-MT-TK + G23-MT-TK | AGKS |
| LT51-Mt-tk + RB38-Mt-tk | AGKS |
| LB510-Nd1 + RB54-Nd1 | AGKS |
| G35-R6c + G28-R6c | AGKS |

TABLE 12
Truncations - N-terminal truncation of DddAC
(FIG. 53D and FIG. 72E-72F)

| | | | SEQ |
|---|---|---|---|
| | | | ID |
| Name | Length | Sequence | NO: |
| Canonical | 30 | AIPVKRGATGETKVFI | 920 |
| (T1413I) | | GNSNSPKSPTKGGC | |
| NΔ1 | 29 | IPVKRGATGETKVFIG | 921 |
| | | NSNSPKSPTKGGC | |
| NΔ2 | 28 | PVKRGATGETKVFIG | 922 |
| | | NSNSPKSPTKGGC | |
| NΔ3 | 27 | VKRGATGETKVFIGN | 923 |
| | | SNSPKSPTKGGC | |
| NΔ4 | 26 | KRGATGETKVFIGNS | 924 |
| | | NSPKSPTKGGC | |
| NΔ5 | 25 | RGATGETKVFIGNSN | 925 |
| | | SPKSPTKGGC | |
| NΔ6 | 24 | GATGETKVFIGNSNS | 926 |
| | | PKSPTKGGC | |
| NΔ7 | 23 | ATGETKVFIGNSNSP | 927 |
| | | KSPTKGGC | |
| NΔ8 | 22 | TGETKVFIGNSNSPKS | 928 |
| | | PTKGGC | |
| NΔ9 | 21 | GETKVFIGNSNSPKSP | 929 |
| | | TKGGC | |
| NΔ10 | 20 | ETKVFIGNSNSPKSPT | 930 |
| | | KGGC | |
| NΔ11 | 19 | TKVFIGNSNSPKSPTK | 931 |
| | | GGC | |
| NΔ12 | 18 | KVFIGNSNSPKSPTKG | 932 |
| | | GC | |
| NΔ13 | 17 | VFIGNSNSPKSPTKGGC | 933 |
| NΔ14 | 16 | FIGNSNSPKSPTKGGC | 934 |
| NΔ15 | 15 | IGNSNSPKSPTKGGC | 935 |

TABLE 13
Truncations - C-terminal truncation of DddAC
(FIG. 53D and FIGS. 72G-72H)

| Name | Length | Sequence | SEQ ID NO: |
|---|---|---|---|
| Canonical | 30 | AIPVKRGATGETKVFI | 920 |
| (T1413I) | | GNSNSPKSPTKGGC | |
| CΔ1 | 29 | AIPVKRGATGETKVFI | 936 |
| | | GNSNSPKSPTKGG | |
| CΔ2 | 28 | AIPVKRGATGETKVFI | 937 |
| | | GNSNSPKSPTKG | |
| CΔ3 | 27 | AIPVKRGATGETKVFI | 938 |
| | | GNSNSPKSPTK | |
| CΔ4 | 26 | AIPVKRGATGETKVFI | 939 |
| | | GNSNSPKSPT | |
| CΔ5 | 25 | AIPVKRGATGETKVFI | 940 |
| | | GNSNSPKSP | |
| CΔ6 | 24 | AIPVKRGATGETKVFI | 941 |
| | | GNSNSPKS | |
| CΔ7 | 23 | AIPVKRGATGETKVFI | 942 |
| | | GNSNSPK | |
| CΔ8 | 22 | AIPVKRGATGETKVFI | 943 |
| | | GNSNSP | |
| CΔ9 | 21 | AIPVKRGATGETKVFI | 944 |
| | | GNSNS | |

TABLE 14
Truncations - C-terminal truncation of DddAN
(FIG. 53D and FIGS. 72E-72H)

| | | | SEQ |
|---|---|---|---|
| Name | Length | Sequence | ID NO: |
| Canonical | 108 | GSYALGPYQISAPQ | 945 |
| (T1380I,E1396K) | | LPAYNGQTVGTFYY | |
| | | VNDAGGLESKVFSS | |
| | | GGPTPYPNYANAG | |
| | | HVEGQSALFMRDN | |
| | | GISEGLVFHNNPEG | |
| | | TCGFCVNMIETLLP | |
| | | ENAKMTVVPPKG | |
| CΔ1 | 107 | GSYALGPYQISAPQ | 946 |
| | | LPAYNGQTVGTFYY | |
| | | VNDAGGLESKVFSS | |
| | | GGPTPYPNYANAG | |
| | | HVEGQSALFMRDN | |
| | | GISEGLVFHNNPEG | |
| | | TCGFCVNMIETLLP | |
| | | ENAKMTVVPPK | |
| CΔ2 | 106 | GSYALGPYQISAPQ | 947 |
| | | LPAYNGQTVGTFYY | |
| | | VNDAGGLESKVFSS | |
| | | GGPTPYPNYANAG | |
| | | HVEGQSALFMRDN | |
| | | GISEGLVFHNNPEG | |
| | | TCGFCVNMIETLLP | |
| | | ENAKMTVVPP | |
| CΔ3 | 105 | GSYALGPYQISAPQ | 948 |
| | | LPAYNGQTVGTFYY | |
| | | VNDAGGLESKVFSS | |
| | | GGPTPYPNYANAG | |
| | | HVEGQSALFMRDN | |
| | | GISEGLVFHNNPEG | |
| | | TCGFCVNMIETLLP | |
| | | ENAKMTVVP | |
| CΔ4 | 104 | GSYALGPYQISAPQ | 949 |
| | | LPAYNGQTVGTFYY | |
| | | VNDAGGLESKVFSS | |
| | | GGPTPYPNYANAG | |
| | | HVEGQSALFMRDN | |
| | | GISEGLVFHNNPEG | |
| | | TCGFCVNMIETLLP | |
| | | ENAKMTVV | |
| CΔ5 | 103 | GSYALGPYQISAPQ | 950 |
| | | LPAYNGQTVGTFYY | |
| | | VNDAGGLESKVFSS | |
| | | GGPTPYPNYANAG | |
| | | HVEGQSALFMRDN | |
| | | GISEGLVFHNNPEG | |
| | | TCGFCVNMIETLLP | |
| | | ENAKMTV | |
| CΔ6 | 102 | GSYALGPYQISAPQ | 951 |
| | | LPAYNGQTVGTFYY | |
| | | VNDAGGLESKVFSS | |
| | | GGPTPYPNYANAG | |
| | | HVEGQSALFMRDN | |
| | | GISEGLVFHNNPEG | |
| | | TCGFCVNMIETLLP | |
| | | ENAKMT | |

TABLE 15
Truncations - C-terminal extension of DddAN
(FIGS. 73A-73B)

| | | | SEQ |
|---|---|---|---|
| | | | ID |
| Name | Length | Sequence | NO: |
| Canonical | 108 | GSYALGPYQISAPQL | 945 |
| (T1380I, | | PAYNGQTVGTFYYV | |
| E1396K) | | NDAGGLESKVFSSG | |
| | | GPTPYPNYANAGHV | |
| | | EGQSALFMRDNGISE | |
| | | GLVFHNNPEGTCGFC | |
| | | VNMIETLLPENAKM | |
| | | TVVPPKG | |
| C+1 | 109 | GSYALGPYQISAPQL | 951 |
| | | PAYNGQTVGTFYYV | |
| | | NDAGGLESKVFSSG | |
| | | GPTPYPNYANAGHV | |
| | | EGQSALFMRDNGISE | |
| | | GLVFHNNPEGTCGFC | |
| | | VNMIETLLPENAKM | |
| | | TVVPPKGA | |
| C+2 | 110 | GSYALGPYQISAPQL | 952 |
| | | PAYNGQTVGTFYYV | |
| | | NDAGGLESKVFSSG | |
| | | GPTPYPNYANAGHV | |
| | | EGQSALFMRDNGISE | |
| | | GLVFHNNPEGTCGFC | |
| | | VNMIETLLPENAKM | |
| | | TVVPPKGAI | |
| C+3 | 111 | GSYALGPYQISAPQL | 953 |
| | | PAYNGQTVGTFYYV | |
| | | NDAGGLESKVFSSG | |
| | | GPTPYPNYANAGHV | |
| | | EGQSALFMRDNGISE | |
| | | GLVFHNNPEGTCGFC | |
| | | VNMIETLLPENAKM | |
| | | TVVPPKGAIP | |
| C+4 | 112 | GSYALGPYQISAPQL | 954 |
| | | PAYNGQTVGTFYYV | |
| | | NDAGGLESKVFSSG | |
| | | GPTPYPNYANAGHV | |
| | | EGQSALFMRDNGISE | |
| | | GLVFHNNPEGTCGFC | |
| | | VNMIETLLPENAKM | |
| | | TVVPPKGAIPV | |
| C+5 | 113 | GSYALGPYQISAPQL | 955 |
| | | PAYNGQTVGTFYYV | |
| | | NDAGGLESKVFSSG | |
| | | GPTPYPNYANAGHV | |
| | | EGQSALFMRDNGISE | |
| | | GLVFHNNPEGTCGFC | |
| | | VNMIETLLPENAKM | |
| | | TVVPPKGAIPVK | |
| C+6 | 114 | GSYALGPYQISAPQL | 956 |
| | | PAYNGQTVGTFYYV | |
| | | NDAGGLESKVFSSG | |
| | | GPTPYPNYANAGHV | |
| | | EGQSALFMRDNGISE | |
| | | GLVFHNNPEGTCGFC | |
| | | VNMIETLLPENAKM | |
| | | TVVPPKGAIPVKR | |
| C+7 | 115 | GSYALGPYQISAPQL | 957 |
| | | PAYNGQTVGTFYYV | |
| | | NDAGGLESKVFSSG | |
| | | GPTPYPNYANAGHV | |
| | | EGQSALFMRDNGISE | |
| | | GLVFHNNPEGTCGFC | |
| | | VNMIETLLPENAKM | |
| | | TVVPPKGAIPVKRG | |
| C+8 | 116 | GSYALGPYQISAPQL | 958 |
| | | PAYNGQTVGTFYYV | |
| | | NDAGGLESKVFSSG | |
| | | GPTPYPNYANAGHV | |
| | | EGQSALFMRDNGISE | |
| | | GLVFHNNPEGTCGFC | |
| | | VNMIETLLPENAKM | |
| | | TVVPPKGAIPVKRGA | |
| C+9 | 117 | GSYALGPYQISAPQL | 959 |
| | | PAYNGQTVGTFYYV | |
| | | NDAGGLESKVFSSG | |
| | | GPTPYPNYANAGHV | |
| | | EGQSALFMRDNGISE | |
| | | GLVFHNNPEGTCGFC | |
| | | VNMIETLLPENAKM | |
| | | TVVPPKGAIPVKRGA | |
| | | T | |
| C+10 | 118 | GSYALGPYQISAPQL | 960 |
| | | PAYNGQTVGTFYYV | |
| | | NDAGGLESKVFSSG | |
| | | GPTPYPNYANAGHV | |
| | | EGQSALFMRDNGISE | |
| | | GLVFHNNPEGTCGFC | |
| | | VNMIETLLPENAKM | |
| | | TVVPPKGAIPVKRGA | |
| | | TG | |
| C+11 | 119 | GSYALGPYQISAPQL | 961 |
| | | PAYNGQTVGTFYYV | |
| | | NDAGGLESKVFSSG | |
| | | GPTPYPNYANAGHV | |
| | | EGQSALFMRDNGISE | |
| | | GLVFHNNPEGTCGFC | |
| | | VNMIETLLPENAKM | |
| | | TVVPPKGAIPVKRGA | |
| | | TGE | |
| C+12 | 120 | GSYALGPYQISAPQL | 962 |
| | | PAYNGQTVGTFYYV | |
| | | NDAGGLESKVFSSG | |
| | | GPTPYPNYANAGHV | |
| | | EGQSALFMRDNGISE | |
| | | GLVFHNNPEGTCGFC | |
| | | VNMIETLLPENAKM | |
| | | TVVPPKGAIPVKRGA | |
| | | TGET | |
| C+13 | 121 | GSYALGPYQISAPQL | 963 |
| | | PAYNGQTVGTFYYV | |
| | | NDAGGLESKVFSSG | |
| | | GPTPYPNYANAGHV | |
| | | EGQSALFMRDNGISE | |
| | | GLVFHNNPEGTCGFC | |
| | | VNMIETLLPENAKM | |
| | | TVVPPKGAIPVKRGA | |
| | | TGETK | |
| C+14 | 122 | GSYALGPYQISAPQL | 964 |
| | | PAYNGQTVGTFYYV | |
| | | NDAGGLESKVFSSG | |
| | | GPTPYPNYANAGHV | |
| | | EGQSALFMRDNGISE | |
| | | GLVFHNNPEGTCGFC | |
| | | VNMIETLLPENAKM | |
| | | TVVPPKGAIPVKRGA | |
| | | TGETKV | |
| C+15 | 123 | GSYALGPYQISAPQL | 965 |
| | | PAYNGQTVGTFYYV | |
| | | NDAGGLESKVFSSG | |
| | | GPTPYPNYANAGHV | |
| | | EGQSALFMRDNGISE | |
| | | GLVFHNNPEGTCGFC | |
| | | VNMIETLLPENAKM | |
| | | TVVPPKGAIPVKRGA | |
| | | TGETKVF | |

TABLE 16
DddA Point Mutations - Ala point mutations
(FIG. 53E)

| | | SEQ | |
|---|---|---|---|
| | | ID | |
| Name | Sequence | NO: | Length |
| Canon- | AIPVKRGATGETKVFIGNSNSPKSPTKGGC | 967 | 30 |
| ical | | | |
| (T1413I) | | | |
| I2A | AAPVKRGATGETKVFIGNSNSPKSPTKGGC | 968 | 30 |
| P3A | AIAVKRGATGETKVFIGNSNSPKSPTKGGC | 969 | 30 |
| V4A | AIPAKRGATGETKVFIGNSNSPKSPTKGGC | 970 | 30 |
| K5A | AIPVARGATGETKVFIGNSNSPKSPTKGGC | 971 | 30 |
| R6A | AIPVKAGATGETKVFIGNSNSPKSPTKGGC | 972 | 30 |
| G7A | AIPVKRAATGETKVFIGNSNSPKSPTKGGC | 973 | 30 |
| T9A | AIPVKRGAAGETKVFIGNSNSPKSPTKGGC | 974 | 30 |
| G10A | AIPVKRGATAETKVFIGNSNSPKSPTKGGC | 975 | 30 |
| E11A | AIPVKRGATGATKVFIGNSNSPKSPTKGGC | 976 | 30 |
| T12A | AIPVKRGATGEAKVFIGNSNSPKSPTKGGC | 977 | 30 |
| K13A | AIPVKRGATGETAVFIGNSNSPKSPTKGGC | 978 | 30 |
| V14A | AIPVKRGATGETKAFIGNSNSPKSPTKGGC | 979 | 30 |
| F15A | AIPVKRGATGETKVAIGNSNSPKSPTKGGC | 980 | 30 |
| T16A | AIPVKRGATGETKVFAGNSNSPKSPTKGGC | 981 | 30 |
| G17A | AIPVKRGATGETKVFIANSNSPKSPTKGGC | 982 | 30 |
| N18A | AIPVKRGATGETKVFIGASNSPKSPTKGGC | 983 | 30 |
| S19A | AIPVKRGATGETKVFIGNANSPKSPTKGGC | 984 | 30 |
| N20A | AIPVKRGATGETKVFIGNSASPKSPTKGGC | 985 | 30 |
| S21A | AIPVKRGATGETKVFIGNSNAPKSPTKGGC | 986 | 30 |
| P22A | AIPVKRGATGETKVFIGNSNSAKSPTKGGC | 987 | 30 |
| K23A | AIPVKRGATGETKVFIGNSNSPASPTKGGC | 988 | 30 |
| S24A | AIPVKRGATGETKVFIGNSNSPKAPTKGGC | 989 | 30 |
| P25A | AIPVKRGATGETKVFIGNSNSPKSATKGGC | 990 | 30 |
| T26A | AIPVKRGATGETKVFIGNSNSPKSPAKGGC | 991 | 30 |
| K27A | AIPVKRGATGETKVFIGNSNSPKSPTAGGC | 992 | 30 |
| G28A | AIPVKRGATGETKVFIGNSNSPKSPTKAGC | 993 | 30 |
| G29A | AIPVKRGATGETKVFIGNSNSPKSPTKGAC | 994 | 30 |
| C30A | AIPVKRGATGETKVFIGNSNSPKSPTKGGA | 995 | 30 |

TABLE 17
DddA Point Mutations - Lys point mutations (FIG. 53F)

| | | SEQ ID | |
|---|---|---|---|
| Name | Sequence | NO: | Length |
| Canonical (T1413I) | AIPVKRGATGETKVFIGNSNSPKSPTKGGC | 996 | 30 |
| A1K | KIPVKRGATGETKVFIGNSNSPKSPTKGGC | 997 | 30 |
| I2K | AKPVKRGATGETKVFIGNSNSPKSPTKGGC | 998 | 30 |
| P3K | AIKVKRGATGETKVFIGNSNSPKSPTKGGC | 999 | 30 |
| V4K | AIPKKRGATGETKVFIGNSNSPKSPTKGGC | 1000 | 30 |
| R6K | AIPVKKGATGETKVFIGNSNSPKSPTKGGC | 1001 | 30 |
| G7K | AIPVKRKATGETKVFIGNSNSPKSPTKGGC | 1002 | 30 |
| A8K | AIPVKRGKTGETKVFIGNSNSPKSPTKGGC | 1003 | 30 |
| T9K | AIPVKRGAKGETKVFIGNSNSPKSPTKGGC | 1004 | 30 |
| G10K | AIPVKRGATKETKVFIGNSNSPKSPTKGGC | 1005 | 30 |
| E11K | AIPVKRGATGKTKVFIGNSNSPKSPTKGGC | 1006 | 30 |
| T12K | AIPVKRGATGEKKVFIGNSNSPKSPTKGGC | 1007 | 30 |
| V14K | AIPVKRGATGETKKFIGNSNSPKSPTKGGC | 1008 | 30 |
| F15K | AIPVKRGATGETKVKIGNSNSPKSPTKGGC | 1009 | 30 |
| T16K | AIPVKRGATGETKVFKGNSNSPKSPTKGGC | 1010 | 30 |
| G17K | AIPVKRGATGETKVFIKNSNSPKSPTKGGC | 1011 | 30 |
| N18K | AIPVKRGATGETKVFIGKSNSPKSPTKGGC | 1012 | 30 |
| S19K | AIPVKRGATGETKVFIGNKNSPKSPTKGGC | 1013 | 30 |
| N20K | AIPVKRGATGETKVFIGNSKSPKSPTKGGC | 1014 | 30 |
| S21K | AIPVKRGATGETKVFIGNSNKPKSPTKGGC | 1015 | 30 |
| P22K | AIPVKRGATGETKVFIGNSNSKKSPTKGGC | 1016 | 30 |
| S24K | AIPVKRGATGETKVFIGNSNSPKKPTKGGC | 1017 | 30 |
| P25K | AIPVKRGATGETKVFIGNSNSPKSKTKGGC | 1018 | 30 |
| T26K | AIPVKRGATGETKVFIGNSNSPKSPKKGGC | 1019 | 30 |
| G28K | AIPVKRGATGETKVFIGNSNSPKSPTKKGC | 1020 | 30 |
| G29K | AIPVKRGATGETKVFIGNSNSPKSPTKGKC | 1021 | 30 |
| C30K | AIPVKRGATGETKVFIGNSNSPKSPTKGGK | 1022 | 30 |

TABLE 18
DddA Point Mutations - Asp point mutations (FIG. 53G)

| | | SEQ ID | |
|---|---|---|---|
| Name | Sequence | NO: | Length |
| Canonical (T1413I) | AIPVKRGATGETKVFIGNSNSPKSPTKGGC | 1023 | 30 |
| A1D | DIPVKRGATGETKVFIGNSNSPKSPTKGGC | 1024 | 30 |
| I2D | ADPVKRGATGETKVFIGNSNSPKSPTKGGC | 1025 | 30 |
| P3D | AIDVKRGATGETKVFIGNSNSPKSPTKGGC | 1026 | 30 |
| V4D | AIPDKRGATGETKVFIGNSNSPKSPTKGGC | 1027 | 30 |
| K5D | AIPVDRGATGETKVFIGNSNSPKSPTKGGC | 1028 | 30 |
| R6D | AIPVKDGATGETKVFIGNSNSPKSPTKGGC | 1029 | 30 |
| G7D | AIPVKRDATGETKVFIGNSNSPKSPTKGGC | 1030 | 30 |
| A8D | AIPVKRGDTGETKVFIGNSNSPKSPTKGGC | 1031 | 30 |
| T9D | AIPVKRGADGETKVFIGNSNSPKSPTKGGC | 1032 | 30 |
| G10D | AIPVKRGATDETKVFIGNSNSPKSPTKGGC | 1033 | 30 |
| E11D | AIPVKRGATGDTKVFIGNSNSPKSPTKGGC | 1034 | 30 |
| T12D | AIPVKRGATGEDKVFIGNSNSPKSPTKGGC | 1035 | 30 |
| K13D | AIPVKRGATGETDVFIGNSNSPKSPTKGGC | 1036 | 30 |
| V14D | AIPVKRGATGETKDFIGNSNSPKSPTKGGC | 1037 | 30 |
| F15D | AIPVKRGATGETKVDIGNSNSPKSPTKGGC | 1038 | 30 |
| T16D | AIPVKRGATGETKVFDGNSNSPKSPTKGGC | 1039 | 30 |
| G17D | AIPVKRGATGETKVFIDNSNSPKSPTKGGC | 1040 | 30 |
| N18D | AIPVKRGATGETKVFIGDSNSPKSPTKGGC | 1041 | 30 |
| S19D | AIPVKRGATGETKVFIGNDNSPKSPTKGGC | 1042 | 30 |
| N20D | AIPVKRGATGETKVFIGNSDSPKSPTKGGC | 1043 | 30 |
| S21D | AIPVKRGATGETKVFIGNSNDPKSPTKGGC | 1044 | 30 |
| P22D | AIPVKRGATGETKVFIGNSNSDKSPTKGGC | 1045 | 30 |
| K23D | AIPVKRGATGETKVFIGNSNSPDSPTKGGC | 1046 | 30 |
| S24D | AIPVKRGATGETKVFIGNSNSPKDPTKGGC | 1047 | 30 |
| P25D | AIPVKRGATGETKVFIGNSNSPKSDTKGGC | 1048 | 30 |
| T26D | AIPVKRGATGETKVFIGNSNSPKSPDKGGC | 1049 | 30 |
| K27D | AIPVKRGATGETKVFIGNSNSPKSPTDGGC | 1050 | 30 |
| G28D | AIPVKRGATGETKVFIGNSNSPKSPTKDGC | 1051 | 30 |
| G29D | AIPVKRGATGETKVFIGNSNSPKSPTKGDC | 1052 | 30 |
| C30D | AIPVKRGATGETKVFIGNSNSPKSPTKGGD | 1053 | 30 |

TABLE 19
DddA Point Mutations - Glu point mutations (FIG. 53H)

| | | SEQ ID | |
|---|---|---|---|
| Name | Sequence | NO: | Length |
| Canonical (T1413I) | AIPVKRGATGETKVFIGNSNSPKSPTKGGC | 1054 | 30 |
| A1E | EIPVKRGATGETKVFIGNSNSPKSPTKGGC | 1055 | 30 |
| I2E | AEPVKRGATGETKVFIGNSNSPKSPTKGGC | 1056 | 30 |
| P3E | AIEVKRGATGETKVFIGNSNSPKSPTKGGC | 1057 | 30 |
| V4E | AIPEKRGATGETKVFIGNSNSPKSPTKGGC | 1058 | 30 |
| K5E | AIPVERGATGETKVFIGNSNSPKSPTKGGC | 1059 | 30 |
| R6E | AIPVKEGATGETKVFIGNSNSPKSPTKGGC | 1060 | 30 |
| G7E | AIPVKREATGETKVFIGNSNSPKSPTKGGC | 1061 | 30 |
| A8E | AIPVKRGETGETKVFIGNSNSPKSPTKGGC | 1062 | 30 |
| T9E | AIPVKRGAEGETKVFIGNSNSPKSPTKGGC | 1063 | 30 |
| G10E | AIPVKRGATEETKVFIGNSNSPKSPTKGGC | 1064 | 30 |
| T12E | AIPVKRGATGEEKVFIGNSNSPKSPTKGGC | 1065 | 30 |
| K13E | AIPVKRGATGETEVFIGNSNSPKSPTKGGC | 1066 | 30 |
| V14E | AIPVKRGATGETKEFIGNSNSPKSPTKGGC | 1067 | 30 |
| F15E | AIPVKRGATGETKVEIGNSNSPKSPTKGGC | 1068 | 30 |
| T16E | AIPVKRGATGETKVFEGNSNSPKSPTKGGC | 1069 | 30 |
| G17E | AIPVKRGATGETKVFIENSNSPKSPTKGGC | 1070 | 30 |
| N18E | AIPVKRGATGETKVFIGESNSPKSPTKGGC | 1071 | 30 |
| S19E | AIPVKRGATGETKVFIGNENSPKSPTKGGC | 1072 | 30 |
| N20E | AIPVKRGATGETKVFIGNSESPKSPTKGGC | 1073 | 30 |
| S21E | AIPVKRGATGETKVFIGNSNEPKSPTKGGC | 1074 | 30 |
| P22E | AIPVKRGATGETKVFIGNSNSEKSPTKGGC | 1075 | 30 |
| K23E | AIPVKRGATGETKVFIGNSNSPESPTKGGC | 1076 | 30 |
| S24E | AIPVKRGATGETKVFIGNSNSPKEPTKGGC | 1077 | 30 |
| P25E | AIPVKRGATGETKVFIGNSNSPKSETKGGC | 1078 | 30 |
| T26E | AIPVKRGATGETKVFIGNSNSPKSPEKGGC | 1079 | 30 |
| K27E | AIPVKRGATGETKVFIGNSNSPKSPTEGGC | 1080 | 30 |
| G28E | AIPVKRGATGETKVFIGNSNSPKSPTKEGC | 1081 | 30 |
| G29E | AIPVKRGATGETKVFIGNSNSPKSPTKGEC | 1082 | 30 |
| C30E | AIPVKRGATGETKVFIGNSNSPKSPTKGGE | 1083 | 30 |

TABLE 20
Introducing negative charge at the
termini of DddA (Asp) (FIG. 53I)

| Name | DddA^{N} | DddA^{C} |
|---|---|---|
| Canonical | Canonical | Canonical |
| Canonical_D-3-0 | Canonical | D-3-0 |
| Canonical_D-6-0 | Canonical | D-6-0 |
| Canonical_D-9-0 | Canonical | D-9-0 |
| Canonical_D-3-GS | Canonical | D-3-GS |
| Canonical_D-6-GS | Canonical | D-6-GS |
| Canonical_D-9-GS | Canonical | D-9-GS |
| D-3-0_Canonical | D-3-0 | Canonical |
| D-6-0_Canonical | D-6-0 | Canonical |
| D-9-0_Canonical | D-9-0 | Canonical |
| D-3-GS_Canonical | D-3-GS | Canonical |
| D-6-GS_Canonical | D-6-GS | Canonical |
| D-9-GS_Canonical | D-9-GS | Canonical |
| endD-3-0_Canonical | endD-3-0 | Canonical |
| endD-6-0_Canonical | endD-6-0 | Canonical |
| endD-9-0_Canonical | endD-9-0 | Canonical |
| endD-3-SG_Canonical | endD-3-SG | Canonical |
| endD-6-SG_Canonical | endD-6-SG | Canonical |
| endD-9-SG_Canonical | endD-9-SG | Canonical |
| D-3-0_D-3-0 | D-3-0 | D-3-0 |
| D-6-0_D-6-0 | D-6-0 | D-6-0 |
| D-9-0_D-9-0 | D-9-0 | D-9-0 |
| D-3-GS_D-3-GS | D-3-GS | D-3-GS |
| D-6-GS_D-6-GS | D-6-GS | D-6-GS |
| D-9-GS_D-9-GS | D-9-GS | D-9-GS |
| endD-3-0_D-3-0 | endD-3-0 | D-3-0 |
| endD-6-0_D-6-0 | endD-6-0 | D-6-0 |
| endD-9-0_D-9-0 | endD-9-0 | D-9-0 |
| endD-3-SG_D-3-GS | endD-3-SG | D-3-GS |
| endD-6-SG_D-6-GS | endD-6-SG | D-6-GS |
| endD-9-SG_D-9-GS | endD-9-SG | D-9-GS |

TABLE 21
Introducing negative charge at the
termini of DddA (Glu) (FIG. 53J)

| Name | DddA^{N} | DddA^{C} |
|---|---|---|
| Canonical | Canonical | Canonical |
| Canonical_E-3-0 | Canonical | E-3-0 |
| Canonical_E-6-0 | Canonical | E-6-0 |
| Canonical_E-9-0 | Canonical | E-9-0 |
| Canonical_E-3-GS | Canonical | E-3-GS |
| Canonical_E-6-GS | Canonical | E-6-GS |
| Canonical_E-9-GS | Canonical | E-9-GS |
| E-3-0_Canonical | E-3-0 | Canonical |
| E-6-0_Canonical | E-6-0 | Canonical |
| E-9-0_Canonical | E-9-0 | Canonical |
| E-3-GS_Canonical | E-3-GS | Canonical |
| E-6-GS_Canonical | E-6-GS | Canonical |
| E-9-GS_Canonical | E-9-GS | Canonical |
| endE-3-0_Canonical | endE-3-0 | Canonical |
| endE-6-0_Canonical | endE-6-0 | Canonical |
| endE-9-0_Canonical | endE-9-0 | Canonical |
| endE-3-SG_Canonical | endE-3-SG | Canonical |
| endE-6-SG_Canonical | endE-6-SG | Canonical |
| endE-9-SG_Canonical | endE-9-SG | Canonical |
| E-3-0_E-3-0 | E-3-0 | E-3-0 |
| E-6-0_E-6-0 | E-6-0 | E-6-0 |
| E-9-0_E-9-0 | E-9-0 | E-9-0 |
| E-3-GS_E-3-GS | E-3-GS | E-3-GS |
| E-6-GS_E-6-GS | E-6-GS | E-6-GS |
| E-9-GS_E-9-GS | E-9-GS | E-9-GS |
| endE-3-0_E-3-0 | endE-3-0 | E-3-0 |
| endE-6-0_E-6-0 | endE-6-0 | E-6-0 |
| endE-9-0_E-9-0 | endE-9-0 | E-9-0 |
| endE-3-SG_E-3-GS | endE-3-SG | E-3-GS |
| endE-6-SG_E-6-GS | endE-6-SG | E-6-GS |
| endE-9-SG_E-9-GS | endE-9-SG | E-9-GS |

TABLE 22
Replace the 13-amino acid Gly/Ser-rich flexible
linker between the ZF array and either DddAN
or DddAC with the following sequences.

| | | | SEQ |
|---|---|---|---|
| Name | Length | Sequence | ID NO: |
| Canonical | 13 | GSGGGGSGGSGGS | 309 |
| D-3-0 | 13 | GSGGGGSGGSDDD | 316 |
| D-6-0 | 13 | GSGGGGSDDDDDD | 317 |
| D-9-0 | 13 | GSGGDDDDDDDDD | 318 |
| D-3-GS | 13 | GSGGGGSGDDDGS | 319 |
| D-6-GS | 13 | GSGGGDDDDDDGS | 320 |
| D-9-GS | 13 | GSDDDDDDDDDGS | 321 |
| E-3-0 | 13 | GSGGGGSGGSEEE | 310 |
| E-6-0 | 13 | GSGGGGSEEEEEE | 311 |
| E-9-0 | 13 | GSGGEEEEEEEEE | 312 |
| E-3-GS | 13 | GSGGGGSGEEEGS | 313 |
| E-6-GS | 13 | GSGGGEEEEEEGS | 314 |
| E-9-GS | 13 | GSEEEEEEEEEGS | 315 |

TABLE 23
Replace the 4-amino acid Gly/Ser-rich flexible
linker between DddAN and UGI with
the following sequences.

| | | | SEQ |
|---|---|---|---|
| Name | Length | Sequence | ID NO: |
| Canonical | 4 | SGGS | 322 |
| endD-3-0 | 5 | DDDGS | 323 |
| endD-6-0 | 8 | DDDDDDGS | 324 |
| endD-9-0 | 11 | DDDDDDDDDGS | 325 |
| endD-3-SG | 7 | SGDDDGS | 326 |
| endD-6-SG | 10 | SGDDDDDDGS | 327 |
| endD-9-SG | 13 | SGDDDDDDDDDGS | 328 |
| endE-3-0 | 5 | EEEGS | 329 |
| endE-6-0 | 8 | EEEEEEGS | 330 |
| endE-9-0 | 11 | EEEEEEEEEGS | 331 |
| endE-3-SG | 7 | SGEEEGS | 332 |
| endE-6-SG | 10 | SGEEEEEEGS | 333 |
| endE-9-SG | 13 | SGEEEEEEEEEGS | 334 |

TABLE 24
Capping with catalytically inactivated DddAN (FIG. 53K)

| Name | Sequence (N- to C-terminus) | | | | | | | | | |
|---|---|---|---|---|---|---|---|---|---|---|
| Canonical | MTS | FLAG | NES | ZF | 13-aa Gly/Ser-rich | DddA^{C} | 4-aa | UGI | | |
| | | tag | (x2) | array | flexible linker | | linker | | | |
| postUGILink6dDddA | MTS | FLAG | NES | ZF | 13-aa Gly/Ser-rich | DddA^{C} | 4-aa | UGI | Link6 | dDddA |
| | | tag | (x2) | array | flexible linker | | linker | | | |
| postUGILink13dDddA | MTS | FLAG | NES | ZF | 13-aa Gly/Ser-rich | DddA^{C} | 4-aa | UGI | Link13 | dDddA |
| | | tag | (x2) | array | flexible linker | | linker | | | |
| postUGILink20dDddA | MTS | FLAG | NES | ZF | 13-aa Gly/Ser-rich | DddA^{C} | 4-aa | UGI | Link20 | dDddA |
| | | tag | (x2) | array | flexible linker | | linker | | | |
| preUGILink6dDddA | MTS | FLAG | NES | ZF | 13-aa Gly/Ser-rich | DddA^{C} | Link6 | dDddA | Link4 | UGI |
| | | tag | (x2) | array | flexible linker | | | | | |
| preUGILink13dDddA | MTS | FLAG | NES | ZF | 13-aa Gly/Ser-rich | DddA^{C} | Link13 | dDddA | Link4 | UGI |
| | | tag | (x2) | array | flexible linker | | | | | |
| postUGILink6dDddI2K | MTS | FLAG | NES | ZF | 13-aa Gly/Ser-rich | DddA^{C} | 4-aa | UGI | Link6 | dDddI2K |
| | | tag | (x2) | array | flexible linker | | linker | | | |
| postUGILink13dDddI2K | MTS | FLAG | NES | ZF | 13-aa Gly/Ser-rich | DddA^{C} | 4-aa | UGI | Link13 | dDddI2K |
| | | tag | (x2) | array | flexible linker | | linker | | | |
| postUGILink20dDddI2K | MTS | FLAG | NES | ZF | 13-aa Gly/Ser-rich | DddA^{C} | 4-aa | UGI | Link20 | dDddI2K |
| | | tag | (x2) | array | flexible linker | | linker | | | |
| preUGILink6dDddI2K | MTS | FLAG | NES | ZF | 13-aa Gly/Ser-rich | DddA^{C} | Link6 | dDddI2K | Link4 | UGI |
| | | tag | (x2) | array | flexible linker | | | | | |
| preUGILink13dDddI2K | MTS | FLAG | NES | ZF | 13-aa Gly/Ser-rich | DddA^{C} | Link13 | dDddI2K | Link4 | UGI |
| | | tag | (x2) | array | flexible linker | | | | | |

TABLE 25
Capping

| | | | SEQ |
|---|---|---|---|
| | | | ID |
| Name | Length | Sequence | NO: |
| Link6 | 6 | GGSGGS | 1084 |
| Link13 | 13 | GSGGGGSGGSGGS | 309 |
| Link20 | 20 | GSGGGSGGSGGGGSGG | 1084 |
| | | SGGS | |
| dDddA [dDddA^{N} | 108 | GSYALGPYQISAPQLPA | 335 |
| (E1347A)] | | YNGQTVGTFYYVNDAG | |
| | | GLESKVFSSGGPTPYPN | |
| | | YANAGHVAGQSALFMR | |
| | | DNGISEGLVFHNNPEGT | |
| | | CGFCVNMTETLLPENA | |
| | | KMTVVPPEG | |
| dDddI2K [dDddA^{N} | 108 | GSYALGPYQISAPQLPA | 1086 |
| (E1347A,T1380I, | | YNGQTVGTFYYVNDAG | |
| E1396K)] | | GLESKVFSSGGPTPYPN | |
| | | YANAGHVAGQSALFMR | |
| | | DNGISEGLVFHNNPEGT | |
| | | CGFCVNMIETLLPENAK | |
| | | MTVVPPKG | |

TABLE 26
Combining Approaches (FIG. 53L)

| Name | DddA^{N} | DddA^{C} | Comments |
|---|---|---|---|
| Canonical | Canonical | Canonical | |
| CΔ3_Canonical | CΔ3 | Canonical | |
| Canonical_NΔ5 | Canonical | NΔ5 | |
| CΔ3_NΔ5 | CΔ3 | NΔ5 | |
| Canonical_P25A | Canonical | P25A | |
| Canonical_N20E | Canonical | N20E | |
| Canonical_N18K | Canonical | N18K | HS1 |
| Canonical_P25K | Canonical | P25K | |
| Canonical_N18K, P25A | Canonical | N18K, P25A | HS2 |
| Canonical_N18K, P25K | Canonical | N18K, P25K | HS3 |
| Canonical_NΔ5, N18K | Canonical | NΔ5, N18K | |
| Canonical_NΔ5, P25K | Canonical | NΔ5, P25K | |
| CΔ3_P25A | CΔ3 | P25A | |
| CΔ3_N20E | CΔ3 | N20E | |
| CΔ3_N18K | CΔ3 | N18K | |
| CΔ3_P25K | CΔ3 | P25K | |
| CΔ3_N18K, P25A | CΔ3 | N18K, P25A | HS4 |
| CΔ3_N18K, P25K | CΔ3 | N18K, P25K | HS5 |
| CΔ3_NΔ5, N18K | CΔ3 | NΔ5, N18K | |
| CΔ3_NΔ5, P25K | CΔ3 | NΔ5, P25K | |
| Canonical_preUGILink6DddA | Canonical | preUGILink6DddA | |
| Canonical_N20E, preUGILink6DddA | Canonical | N20E, preUGILink6DddA | |
| Canonical_N18K, preUGILink6DddA | Canonical | N18K, preUGILink6DddA | |
| Canonical_P25K, preUGILink6DddA | Canonical | P25K, preUGILink6DddA | |
| Canonical_preUGILink13DddA | Canonical | preUGILink13DddA | |
| Canonical_N20E, preUGILink13DddA | Canonical | N20E, preUGILink13DddA | |
| Canonical_N18K, preUGILink13DddA | Canonical | N18K, preUGILink13DddA | |
| Canonical_P25K, preUGILink13DddA | Canonical | P25K, preUGILink13DddA | |
| CΔ3_preUGILink6DddA | CΔ3 | preUGILink6DddA | |
| CΔ3_N20E, preUGILink6DddA | CΔ3 | N20E, preUGILink6DddA | |
| CΔ3_N18K, preUGILink6DddA | CΔ3 | N18K, preUGILink6DddA | |
| CΔ3_P25K, preUGILink6DddA | CΔ3 | P25K, preUGILink6DddA | |
| CΔ3_preUGILink13DddA | CΔ3 | preUGILink13DddA | |
| CΔ3_N20E, preUGILink13DddA | CΔ3 | N20E, preUGILink13DddA | |
| CΔ3_N18K, preUGILink13DddA | CΔ3 | N18K, preUGILink13DddA | |
| CΔ3_P25K, preUGILink13DddA | CΔ3 | P25K, preUGILink13DddA | |

TABLE 27
Combining Approaches (FIG. 53L)

| Name | DddA^{N} | DddA^{C} |
|---|---|---|
| Canonical | Canonical | Canonical |
| Canonical_K5A | Canonical | K5A |
| Canonical_R6A | Canonical | R6A |
| Canonical_G7A | Canonical | G7A |
| Canonical_T9A | Canonical | T9A |
| Canonical_V14A | Canonical | V14A |
| Canonical_P25A | Canonical | P25A |
| Canonical_T12K | Canonical | T12K |
| Canonical_V14K | Canonical | V14K |
| Canonical_N18K | Canonical | N18K |
| Canonical_P25K | Canonical | P25K |
| Canonical_T12K, V14K | Canonical | T12K, V14K |
| Canonical_T12K, N18K | Canonical | T12K, N18K |
| Canonical_T12K, P25K | Canonical | T12K, P25K |
| Canonical_V14K, N18K | Canonical | V14K, N18K |
| Canonical_V14K, P25K | Canonical | V14K, P25K |
| Canonical_N18K, P25K | Canonical | N18K, P25K |
| Canonical_T12K, V14A | Canonical | T12K, V14A |
| Canonical_T12K, P25A | Canonical | T12K, P25A |
| Canonical_V14A, N18K | Canonical | V14A, N18K |
| Canonical_V14A, P25K | Canonical | V14A, P25K |
| Canonical_V14K, P25A | Canonical | V14K, P25A |
| Canonical_N18K, P25A | Canonical | N18K, P25A |
| Canonical_V14A, P25A | Canonical | V14A, P25A |
| Canonical_G7A, T9A | Canonical | G7A, T9A |
| Canonical_G7A, T12K | Canonical | G7A, T12K |
| Canonical_G7A, V14K | Canonical | G7A, V14K |
| Canonical_G7A, N18K | Canonical | G7A, N18K |
| Canonical_G7A, P25K | Canonical | G7A, P25K |
| Canonical_G7A, V14A | Canonical | G7A, V14A |
| Canonical_G7A, P25A | Canonical | G7A, P25A |
| Canonical_T9A, T12K | Canonical | T9A, T12K |
| Canonical_T9A, V14K | Canonical | T9A, V14K |
| Canonical_T9A, N18K | Canonical | T9A, N18K |
| Canonical_T9A, P25K | Canonical | T9A, P25K |
| Canonical_T9A, V14A | Canonical | T9A, V14A |
| Canonical_T9A, P25A | Canonical | T9A, P25A |
| Canonical_K5A, R6A | Canonical | K5A, R6A |
| Canonical_K5A, G7A | Canonical | K5A, G7A |
| Canonical_K5A, T9A | Canonical | K5A, T9A |
| Canonical_K5A, V14A | Canonical | K5A, V14A |
| Canonical_K5A, P25A | Canonical | K5A, P25A |
| Canonical_K5A, T12K | Canonical | K5A, T12K |
| Canonical_K5A, V14K | Canonical | K5A, V14K |
| Canonical_K5A, N18K | Canonical | K5A, N18K |
| Canonical_K5A, P25K | Canonical | K5A, P25K |
| Canonical_R6A, G7A | Canonical | R6A, G7A |
| Canonical_R6A, T9A | Canonical | R6A, T9A |
| Canonical_R6A, T12K | Canonical | R6A, T12K |
| Canonical_R6A, V14K | Canonical | R6A, V14K |
| Canonical_R6A, N18K | Canonical | R6A, N18K |
| Canonical_R6A, P25K | Canonical | R6A, P25K |
| Canonical_R6A, V14A | Canonical | R6A, V14A |
| Canonical_R6A, P25A | Canonical | R6A, P25A |

TABLE 28
Combining Approaches (FIG. 53L)

| Name | DddA^{N} | DddA^{C} |
|---|---|---|
| Canonical | Canonical | Canonical |
| Canonical_K5A | Canonical | K5A |
| Canonical_R6A | Canonical | R6A |
| Canonical_G7A | Canonical | G7A |
| Canonical_T9A | Canonical | T9A |
| Canonical_V14A | Canonical | V14A |
| Canonical_P25A | Canonical | P25A |
| Canonical_T12K | Canonical | T12K |
| Canonical_V14K | Canonical | V14K |
| Canonical_N18K | Canonical | N18K |
| Canonical_P25K | Canonical | P25K |
| Canonical_T12K, V14K | Canonical | T12K, V14K |
| Canonical_T12K, N18K | Canonical | T12K, N18K |
| Canonical_T12K, P25K | Canonical | T12K, P25K |
| Canonical_V14K, N18K | Canonical | V14K, N18K |
| Canonical_V14K, P25K | Canonical | V14K, P25K |
| Canonical_N18K, P25K | Canonical | N18K, P25K |
| Canonical_T12K, V14A | Canonical | T12K, V14A |
| Canonical_T12K, P25A | Canonical | T12K, P25A |
| Canonical_V14A, N18K | Canonical | V14A, N18K |
| Canonical_V14A, P25K | Canonical | V14A, P25K |
| Canonical_V14K, P25A | Canonical | V14K, P25A |
| Canonical_N18K, P25A | Canonical | N18K, P25A |
| Canonical_V14A, P25A | Canonical | V14A, P25A |
| Canonical_G7A, T9A | Canonical | G7A, T9A |
| Canonical_G7A, T12K | Canonical | G7A, T12K |
| Canonical_G7A, V14K | Canonical | G7A, V14K |
| Canonical_G7A, N18K | Canonical | G7A, N18K |
| Canonical_G7A, P25K | Canonical | G7A, P25K |
| Canonical_G7A, V14A | Canonical | G7A, V14A |
| Canonical_G7A, P25A | Canonical | G7A, P25A |
| Canonical_T9A, T12K | Canonical | T9A, T12K |
| Canonical_T9A, V14K | Canonical | T9A, V14K |
| Canonical_T9A, N18K | Canonical | T9A, N18K |
| Canonical_T9A, P25K | Canonical | T9A, P25K |
| Canonical_T9A, V14A | Canonical | T9A, V14A |
| Canonical_T9A, P25A | Canonical | T9A, P25A |
| Canonical_K5A, R6A | Canonical | K5A, R6A |
| Canonical_K5A, G7A | Canonical | K5A, G7A |
| Canonical_K5A, T9A | Canonical | K5A, T9A |
| Canonical_K5A, V14A | Canonical | K5A, V14A |
| Canonical_K5A, P25A | Canonical | K5A, P25A |
| Canonical_K5A, T12K | Canonical | K5A, T12K |
| Canonical_K5A, V14K | Canonical | K5A, V14K |
| Canonical_K5A, N18K | Canonical | K5A, N18K |
| Canonical_K5A, P25K | Canonical | K5A, P25K |
| Canonical_R6A, G7A | Canonical | R6A, G7A |
| Canonical_R6A, T9A | Canonical | R6A, T9A |
| Canonical_R6A, T12K | Canonical | R6A, T12K |
| Canonical_R6A, V14K | Canonical | R6A, V14K |
| Canonical_R6A, N18K | Canonical | R6A, N18K |
| Canonical_R6A, P25K | Canonical | R6A, P25K |
| Canonical_R6A, V14A | Canonical | R6A, V14A |
| Canonical_R6A, P25A | Canonical | R6A, P25A |
| CΔ3_Canonical | CΔ3 | Canonical |
| CΔ3_K5A | CΔ3 | K5A |
| CΔ3_R6A | CΔ3 | R6A |
| CΔ3_G7A | CΔ3 | G7A |
| CΔ3_T9A | CΔ3 | T9A |
| CΔ3_V14A | CΔ3 | V14A |
| CΔ3_P25A | CΔ3 | P25A |
| CΔ3_T12K | CΔ3 | T12K |
| CΔ3_V14K | CΔ3 | V14K |
| CΔ3_N18K | CΔ3 | N18K |
| CΔ3_P25K | CΔ3 | P25K |
| CΔ3_T12K, V14K | CΔ3 | T12K, V14K |
| CΔ3_T12K, N18K | CΔ3 | T12K, N18K |
| CΔ3_T12K, P25K | CΔ3 | T12K, P25K |
| CΔ3_V14K, N18K | CΔ3 | V14K, N18K |
| CΔ3_V14K, P25K | CΔ3 | V14K, P25K |
| CΔ3_N18K, P25K | CΔ3 | N18K, P25K |
| CΔ3_T12K, V14A | CΔ3 | T12K, V14A |
| CΔ3_T12K, P25A | CΔ3 | T12K, P25A |
| CΔ3_V14A, N18K | CΔ3 | V14A, N18K |
| CΔ3_V14A, P25K | CΔ3 | V14A, P25K |
| CΔ3_V14K, P25A | CΔ3 | V14K, P25A |
| CΔ3_N18K, P25A | CΔ3 | N18K, P25A |
| CΔ3_V14A, P25A | CΔ3 | V14A, P25A |
| CΔ3_G7A, T9A | CΔ3 | G7A, T9A |
| CΔ3_G7A, T12K | CΔ3 | G7A, T12K |
| CΔ3_G7A, V14K | CΔ3 | G7A, V14K |
| CΔ3_G7A, N18K | CΔ3 | G7A, N18K |
| CΔ3_G7A, P25K | CΔ3 | G7A, P25K |
| CΔ3_G7A, V14A | CΔ3 | G7A, V14A |
| CΔ3_G7A, P25A | CΔ3 | G7A, P25A |
| CΔ3_T9A, T12K | CΔ3 | T9A, T12K |
| CΔ3_T9A, V14K | CΔ3 | T9A, V14K |
| CΔ3_T9A, N18K | CΔ3 | T9A, N18K |
| CΔ3_T9A, P25K | CΔ3 | T9A, P25K |
| CΔ3_T9A, V14A | CΔ3 | T9A, V14A |
| CΔ3_T9A, P25A | CΔ3 | T9A, P25A |
| CΔ3_K5A, R6A | CΔ3 | K5A, R6A |
| CΔ3_K5A, G7A | CΔ3 | K5A, G7A |
| CΔ3_K5A, T9A | CΔ3 | K5A, T9A |
| CΔ3_K5A, V14A | CΔ3 | K5A, V14A |
| CΔ3_K5A, P25A | CΔ3 | K5A, P25A |
| CΔ3_K5A, T12K | CΔ3 | K5A, T12K |
| CΔ3_K5A, V14K | CΔ3 | K5A, V14K |
| CΔ3_K5A, N18K | CΔ3 | K5A, N18K |
| CΔ3_K5A, P25K | CΔ3 | K5A, P25K |
| CΔ3_R6A, G7A | CΔ3 | R6A, G7A |
| CΔ3_R6A, T9A | CΔ3 | R6A, T9A |
| CΔ3_R6A, T12K | CΔ3 | R6A, T12K |
| CΔ3_R6A, V14K | CΔ3 | R6A, V14K |
| CΔ3_R6A, N18K | CΔ3 | R6A, N18K |
| CΔ3_R6A, P25K | CΔ3 | R6A, P25K |
| CΔ3_R6A, V14A | CΔ3 | R6A, V14A |
| CΔ3_R6A, P25A | CΔ3 | R6A, P25A |

TABLE 29
Combining Approaches (FIG. 53L)

| Name | DddA^{N} | DddA^{C} |
|---|---|---|
| Canonical | Canonical | Canonical |
| Canonical_K5A | Canonical | K5A |
| Canonical_R6A | Canonical | R6A |
| Canonical_G7A | Canonical | G7A |
| Canonical_T9A | Canonical | T9A |
| Canonical_V14A | Canonical | V14A |
| Canonical_P25A | Canonical | P25A |
| Canonical_T12K | Canonical | T12K |
| Canonical_V14K | Canonical | V14K |
| Canonical_N18K | Canonical | N18K |
| Canonical_P25K | Canonical | P25K |
| Canonical_NΔ5 | Canonical | NΔ5 |
| Canonical_NΔ5, R6A | Canonical | NΔ5, R6A |
| Canonical_NΔ5, G7A | Canonical | NΔ5, G7A |
| Canonical_NΔ5, T9A | Canonical | NΔ5, T9A |
| Canonical_NΔ5, V14A | Canonical | NΔ5, V14A |
| Canonical_NΔ5, P25A | Canonical | NΔ5, P25A |
| Canonical_NΔ5, T12K | Canonical | NΔ5, T12K |
| Canonical_NΔ5, V14K | Canonical | NΔ5, V14K |
| Canonical_NΔ5, N18K | Canonical | NΔ5, N18K |
| Canonical_NΔ5, P25K | Canonical | NΔ5, P25K |
| Canonical_NΔ5, T12K, V14K | Canonical | NΔ5, T12K, V14K |
| Canonical_NΔ5, T12K, N18K | Canonical | NΔ5, T12K, N18K |
| Canonical_NΔ5, T12K, P25K | Canonical | NΔ5, T12K, P25K |
| Canonical_NΔ5, V14K, N18K | Canonical | NΔ5, V14K, N18K |
| Canonical_NΔ5, V14K, P25K | Canonical | NΔ5, V14K, P25K |
| Canonical_NΔ5, N18K, P25K | Canonical | NΔ5, N18K, P25K |
| Canonical_NΔ5, T12K, V14A | Canonical | NΔ5, T12K, V14A |
| Canonical_NΔ5, T12K, P25A | Canonical | NΔ5, T12K, P25A |
| Canonical_NΔ5, V14A, N18K | Canonical | NΔ5, V14A, N18K |
| Canonical_NΔ5, V14A, P25K | Canonical | NΔ5, V14A, P25K |
| Canonical_NΔ5, V14K, P25A | Canonical | NΔ5, V14K, P25A |
| Canonical_NΔ5, N18K, P25A | Canonical | NΔ5, N18K, P25A |
| Canonical_NΔ5, V14A, P25A | Canonical | NΔ5, V14A, P25A |
| Canonical_NΔ5, G7A, T9A | Canonical | NΔ5, G7A, T9A |
| Canonical_NΔ5, G7A, T12K | Canonical | NΔ5, G7A, T12K |
| Canonical_NΔ5, G7A, V14K | Canonical | NΔ5, G7A, V14K |
| Canonical_NΔ5, G7A, N18K | Canonical | NΔ5, G7A, N18K |
| Canonical_NΔ5, G7A, P25K | Canonical | NΔ5, G7A, P25K |
| Canonical_NΔ5, G7A, V14A | Canonical | NΔ5, G7A, V14A |
| Canonical_NΔ5, G7A, P25A | Canonical | NΔ5, G7A, P25A |
| Canonical_NΔ5, T9A, T12K | Canonical | NΔ5, T9A, T12K |
| Canonical_NΔ5, T9A, V14K | Canonical | NΔ5, T9A, V14K |
| Canonical_NΔ5, T9A, N18K | Canonical | NΔ5, T9A, N18K |
| Canonical_NΔ5, T9A, P25K | Canonical | NΔ5, T9A, P25K |
| Canonical_NΔ5, T9A, V14A | Canonical | NΔ5, T9A, V14A |
| Canonical_NΔ5, T9A, P25A | Canonical | NΔ5, T9A, P25A |
| CΔ3_Canonical | CΔ3 | Canonical |
| CΔ3_K5A | CΔ3 | K5A |
| CΔ3_R6A | CΔ3 | R6A |
| CΔ3_G7A | CΔ3 | G7A |
| CΔ3_T9A | CΔ3 | T9A |
| CΔ3_V14A | CΔ3 | V14A |
| CΔ3_P25A | CΔ3 | P25A |
| CΔ3_T12K | CΔ3 | T12K |
| CΔ3_V14K | CΔ3 | V14K |
| CΔ3_N18K | CΔ3 | N18K |
| CΔ3_P25K | CΔ3 | P25K |
| CΔ3_NΔ5 | CΔ3 | NΔ5 |
| CΔ3_NΔ5, R6A | CΔ3 | NΔ5, R6A |
| CΔ3_NΔ5, G7A | CΔ3 | NΔ5, G7A |
| CΔ3_NΔ5, T9A | CΔ3 | NΔ5, T9A |
| CΔ3_NΔ5, V14A | CΔ3 | NΔ5, V14A |
| CΔ3_NΔ5, P25A | CΔ3 | NΔ5, P25A |
| CΔ3_NΔ5, T12K | CΔ3 | NΔ5, T12K |
| CΔ3_NΔ5, V14K | CΔ3 | NΔ5, V14K |
| CΔ3_NΔ5, N18K | CΔ3 | NΔ5, N18K |
| CΔ3_NΔ5, P25K | CΔ3 | NΔ5, P25K |
| CΔ3_NΔ5, T12K, V14K | CΔ3 | NΔ5, T12K, V14K |
| CΔ3_NΔ5, T12K, N18K | CΔ3 | NΔ5, T12K, N18K |
| CΔ3_NΔ5, T12K, P25K | CΔ3 | NΔ5, T12K, P25K |
| CΔ3_NΔ5, V14K, N18K | CΔ3 | NΔ5, V14K, N18K |
| CΔ3_NΔ5, V14K, P25K | CΔ3 | NΔ5, V14K, P25K |
| CΔ3_NΔ5, N18K, P25K | CΔ3 | NΔ5, N18K, P25K |
| CΔ3_NΔ5, T12K, V14A | CΔ3 | NΔ5, T12K, V14A |
| CΔ3_NΔ5, T12K, P25A | CΔ3 | NΔ5, T12K, P25A |
| CΔ3_NΔ5, V14A, N18K | CΔ3 | NΔ5, V14A, N18K |
| CΔ3_NΔ5, V14A, P25K | CΔ3 | NΔ5, V14A, P25K |
| CΔ3_NΔ5, V14K, P25A | CΔ3 | NΔ5, V14K, P25A |
| CΔ3_NΔ5, N18K, P25A | CΔ3 | NΔ5, N18K, P25A |
| CΔ3_NΔ5, V14A, P25A | CΔ3 | NΔ5, V14A, P25A |
| CΔ3_NΔ5, G7A, T9A | CΔ3 | NΔ5, G7A, T9A |
| CΔ3_N45, G7A, T12K | CΔ3 | NΔ5, G7A, T12K |
| CΔ3_N45, G7A, V14K | CΔ3 | NΔ5, G7A, V14K |
| CΔ3_N45, G7A, N18K | CΔ3 | NΔ5, G7A, N18K |
| CΔ3_N45, G7A, P25K | CΔ3 | NΔ5, G7A, P25K |
| CΔ3_NΔ5, G7A, V14A | CΔ3 | NΔ5, G7A, V14A |
| CΔ3_NΔ5, G7A, P25A | CΔ3 | NΔ5, G7A, P25A |
| CΔ3_NΔ5, T9A, T12K | CΔ3 | NΔ5, T9A, T12K |
| CΔ3_NΔ5, T9A, V14K | CΔ3 | NΔ5, T9A, V14K |
| CΔ3_N45, T9A, N18K | CΔ3 | N45, T9A, N18K |
| CΔ3_N45, T9A, P25K | CΔ3 | NΔ5, T9A, P25K |
| CΔ3_NΔ5, T9A, V14A | CΔ3 | NΔ5, T9A, V14A |
| CΔ3_NΔ5, T9A, P25A | CΔ3 | NΔ5, T9A, P25A |

TABLE 30
Combining Approaches (FIG. 53L)

| Name | DddA^{N} | DddA^{C} |
|---|---|---|
| Canonical | Canonical | Canonical |
| Canonical_N18K | Canonical | N18K |
| Canonical_R6A, N18K | Canonical | R6A, N18K |
| Canonical_G7A, N18K | Canonical | G7A, N18K |
| Canonical_N18K, P25K | Canonical | N18K, P25K |
| Canonical_preUGILink6DddA | Canonical | preUGILink6DddA |
| Canonical_preUGILink13DddA | Canonical | preUGILink13DddA |
| Canonical_postUGILink20DddA | Canonical | postUGILink20DddA |
| Canonical_N20D, preUGILink6dDddA | Canonical | N20D, preUGILink6dDddA |
| Canonical_N20E, preUGILink6dDddA | Canonical | N20E, preUGILink6dDddA |
| Canonical_N18K, preUGILink6dDddA | Canonical | N18K, preUGILink6dDddA |
| Canonical_P25K, preUGILink6dDddA | Canonical | P25K, preUGILink6dDddA |
| Canonical_N20D, preUGILink13dDddA | Canonical | N20D, preUGILink13dDddA |
| Canonical_N20E, preUGILink13dDddA | Canonical | N20E, preUGILink13dDddA |
| Canonical_N18K, preUGILink13dDddA | Canonical | N18K, preUGILink13dDddA |
| Canonical_P25K, preUGILink13dDddA | Canonical | P25K, preUGILink13dDddA |
| Canonical_N20D, preUGILink20dDddA | Canonical | N20D, preUGILink20dDddA |
| Canonical_N20E, preUGILink20dDddA | Canonical | N20E, preUGILink20dDddA |
| Canonical_N18K, preUGILink20dDddA | Canonical | N18K, preUGILink20dDddA |
| Canonical_P25K, preUGILink20dDddA | Canonical | P25K, preUGILink20dDddA |
| Canonical_N20D, D-9-GS | Canonical | N20D, D-9-GS |
| Canonical_N18K, D-9-GS | Canonical | N18K, D-9-GS |
| Canonical_P25K, D-9-GS | Canonical | P25K, D-9-GS |
| D-6-GS_N20D, D-6-GS | D-6-GS | N20D, D-6-GS |
| D-6-GS_N18K, D-6-GS | D-6-GS | N18K, D-6-GS |
| D-6-GS_P25K, D-6-GS | D-6-GS | P25K, D-6-GS |
| E-6-GS_N20D, E-6-GS | E-6-GS | N20D, E-6-GS |
| E-6-GS_N18K, E-6-GS | E-6-GS | N18K, E-6-GS |
| E-6-GS_P25K, E-6-GS | E-6-GS | P25K, E-6-GS |
| E-6-GS_E-6-GS, postUGILink20dDddI2K | E-6-GS | E-6-GS, postUGILink20dDddI2K |
| endE-6-SG_E-6-GS, postUGILink20dDddI2K | endE-6-SG | E-6-GS, postUGILink20dDddI2K |
| D-6-GS_D-6-GS, preUGILink6dDddA | D-6-GS | D-6-GS, preUGILink6dDddA |
| endD-6-SG_D-6-GS, preUGILink6dDddA | endD-6-SG | D-6-GS, preUGILink6dDddA |
| Canonical_D-9-GS, preUGILink6dDddA | Canonical | D-9-GS, preUGILink6dDddA |
| E-6-GS_E-6-GS, preUGILink6dDddA | E-6-GS | E-6-GS, preUGILink6dDddA |
| endE-6-SG_E-6-GS, preUGILink6dDddA | endE-6-SG | E-6-GS, preUGILink6dDddA |
| D-6-GS_D-6-GS, postUGILink20dDddI2K | D-6-GS | D-6-GS, postUGILink20dDddI2K |
| endD-6-SG_D-6-GS, postUGILink20dDddI2K | endD-6-SG | D-6-GS, postUGILink20dDddI2K |
| Canonical_D-9-GS, postUGILink20dDddI2K | Canonical | D-9-GS, postUGILink20dDddI2K |
| CΔ3_Canonical | CΔ3 | Canonical |
| CΔ3_N18K | CΔ3 | N18K |
| CΔ3_R6A, N18K | CΔ3 | R6A, N18K |
| CΔ3_G7A, N18K | CΔ3 | G7A, N18K |
| CΔ3_N18K, P25K | CΔ3 | N18K, P25K |
| CΔ3_preUGILink6DddA | CΔ3 | preUGILink6DddA |
| CΔ3_preUGILink13DddA | CΔ3 | preUGILink13DddA |
| CΔ3_postUGILink20DddA | CΔ3 | postUGILink20DddA |
| CΔ3_N20D, preUGILink6dDddA | CΔ3 | N20D, preUGILink6dDddA |
| CΔ3_N20E, preUGILink6dDddA | CΔ3 | N20E, preUGILink6dDddA |
| CΔ3_N18K, preUGILink6dDddA | CΔ3 | N18K, preUGILink6dDddA |
| CΔ3_P25K, preUGILink6dDddA | CΔ3 | P25K, preUGILink6dDddA |
| CΔ3_N20D, preUGILink13dDddA | CΔ3 | N20D, preUGILink13dDddA |
| CΔ3_N20E, preUGILink13dDddA | CΔ3 | N20E, preUGILink13dDddA |
| CΔ3_N18K, preUGILink13dDddA | CΔ3 | N18K, preUGILink13dDddA |
| CΔ3_P25K, preUGILink13dDddA | CΔ3 | P25K, preUGILink13dDddA |
| CΔ3_N20D, preUGILink20dDddA | CΔ3 | N20D, preUGILink20dDddA |
| CΔ3_N20E, preUGILink20dDddA | CΔ3 | N20E, preUGILink20dDddA |
| CΔ3_N18K, preUGILink20dDddA | CΔ3 | N18K, preUGILink20dDddA |
| CΔ3_P25K, preUGILink20dDddA | CΔ3 | P25K, preUGILink20dDddA |
| CΔ3_N20D, D-9-GS | CΔ3 | N20D, D-9-GS |
| CΔ3_N18K, D-9-GS | CΔ3 | N18K, D-9-GS |
| CΔ3_P25K, D-9-GS | CΔ3 | P25K, D-9-GS |
| CΔ3_D-9-GS, postUGILink20dDddI2K | CΔ3 | D-9-GS, postUGILink20dDddI2K |
| CΔ3_D-9-GS, preUGILink6dDddA | CΔ3 | D-9-GS, preUGILink6dDddA |

### REFERENCES FOR EXAMPLE 3

1. Mok, B. Y. et al., A bacterial cytidine deaminase toxin enables CRISPR-free mitochondrial base editing. Nature 583, 631-637, doi:10.1038/s41586-020-2477-4 (2020).
2. Mok, B. Y. et al., CRISPR-free base editors with enhanced activity and expanded targeting scope in mitochondrial and nuclear DNA. Nat Biotechnol, doi:10.1038/s41587-022-01256-8 (2022).
3. Kang, B. C. et al., Chloroplast and mitochondrial DNA editing in plants. Nat Plants 7, 899-905, doi:10.1038/s41477-021-00943-9 (2021).
4. Waryah, C. B., Moses, C., Arooj, M. & Blancafort, P. Zinc Fingers, TALEs, and CRISPR Systems: A Comparison of Tools for Epigenome Editing. Methods Mol Biol 1767, 19-63, doi:10.1007/978-1-4939-7774-1_2 (2018).
5. Murphy, E. et al., Mitochondrial Function, Biology, and Role in Disease: A Scientific Statement From the American Heart Association. Circ Res 118, 1960-1991, doi:10.1161/RES.0000000000000104 (2016).
6. Osellame, L. D., Blacker, T. S. & Duchen, M. R. Cellular and molecular mechanisms of mitochondrial function. Best Pract Res Clin Endocrinol Metab 26, 711-723, doi:10.1016/j.beem.2012.05.003 (2012).
7. Reznik, E. et al., Mitochondrial DNA copy number variation across human cancers. Elife 5, doi:10.7554/eLife.10769 (2016).
8. Robin, E. D. & Wong, R. Mitochondrial DNA molecules and virtual number of mitochondria per cell in mammalian cells. J Cell Physiol 136, 507-513, doi:10.1002/jcp.1041360316 (1988).
9. Gorman, G. S. et al., Mitochondrial diseases. Nat Rev Dis Primers 2, 16080, doi:10.1038/nrdp.2016.80 (2016).
10. Stewart, J. B. & Chinnery, P. F. The dynamics of mitochondrial DNA heteroplasmy: implications for human health and disease. Nat Rev Genet 16, 530-542, doi:10.1038/nrg3966 (2015).
11. Lott, M. T. et al., mtDNA Variation and Analysis Using Mitomap and Mitomaster. Curr Protoc Bioinformatics 44, 1 23 21-26, doi:10.1002/0471250953.bi0123s44 (2013).
12. Ryzhkova, A. I. et al., Mitochondrial diseases caused by mtDNA mutations: a mini-review. Ther Clin Risk Manag 14, 1933-1942, doi:10.2147/TCRM.S154863 (2018).
13. Gorman, G. S. et al., Prevalence of nuclear and mitochondrial DNA mutations related to adult mitochondrial disease. Ann Neurol 77, 753-759, doi:10.1002/ana.24362 (2015).
14. Rees, H. A. & Liu, D. R. Base editing: precision chemistry on the genome and transcriptome of living cells. Nat Rev Genet 19, 770-788, doi:10.1038/s41576-018-0059-1 (2018).
15. Huang, T. P., Newby, G. A. & Liu, D. R. Precision genome editing using cytosine and adenine base editors in mammalian cells. Nat Protoc 16, 1089-1128, doi:10.1038/s41596-020-00450-9 (2021).
16. Silva-Pinheiro, P. & Minczuk, M. The potential of mitochondrial genome engineering. Nat Rev Genet 23, 199-214, doi:10.1038/s41576-021-00432-x (2022).
17. Gammage, P. A., Moraes, C. T. & Minczuk, M. Mitochondrial Genome Engineering: The Revolution May Not Be CRISPR-Ized. Trends Genet 34, 101-110, doi:10.1016/j.tig.2017.11.001 (2018).
18. Wiedemann, N. & Pfanner, N. Mitochondrial Machineries for Protein Import and Assembly. Annu Rev Biochem 86, 685-714, doi:10.1146/annurev-biochem-060815-014352 (2017).
19. Mak, A. N., Bradley, P., Bogdanove, A. J. & Stoddard, B. L. TAL effectors: function, structure, engineering and applications. Curr Opin Struct Biol 23, 93-99, doi:10.1016/j.sbi.2012.11.001 (2013).
20. Becker, S. & Boch, J. TALE and TALEN genome editing technologies. Gene Genome Ed 2, 100007 (2021).
21. Andreini, C., Banci, L., Bertini, I. & Rosato, A. Counting the zinc-proteins encoded in the human genome. J Proteome Res 5, 196-201, doi:10.1021/pr050361j (2006).
22. Agustin-Pavon, C., Mielcarek, M., Garriga-Canut, M. & Isalan, M. Deimmunization for gene therapy: host matching of synthetic zinc finger constructs enables long-term mutant Huntingtin repression in mice. Mol Neurodegener 11, 64, doi:10.1186/s13024-016-0128-x (2016).
23. Yang, L. et al., Engineering and optimising deaminase fusions for genome editing. Nat Commun 7, 13330, doi:10.1038/ncomms13330 (2016).
24. Chaudhuri, J. et al., Transcription-targeted DNA deamination by the AID antibody diversification enzyme. Nature 422, 726-730, doi:10.1038/nature01574 (2003).
25. Lim, K., Cho, S. I. & Kim, J. S. Nuclear and mitochondrial DNA editing in human cells with zinc finger deaminases. Nat Commun 13, 366, doi:10.1038/s41467-022-27962-0 (2022).
26. Gammage, P. A., Rorbach, J., Vincent, A. I., Rebar, E. J. & Minczuk, M. Mitochondrially targeted ZFNs for selective degradation of pathogenic mitochondrial genomes bearing large-scale deletions or point mutations. EMBO Mol Med 6, 458-466, doi:10.1002/emmm.201303672 (2014).
27. Minczuk, M., Papworth, M. A., Kolasinska, P., Murphy, M. P. & Klug, A. Sequence-specific modification of mitochondrial DNA using a chimeric zinc finger methylase. Proc Natl Acad Sci USA 103, 19689-19694, doi:10.1073/pnas.0609502103 (2006).
28. Bhakta, M. S. & Segal, D. J. The generation of zinc finger proteins by modular assembly. Methods Mol Biol 649, 3-30, doi:10.1007/978-1-60761-753-2_1 (2010).
29. Gersbach, C. A., Gaj, T. & Barbas, C. F., 3rd. Synthetic zinc finger proteins: the advent of targeted gene regulation and genome modification technologies. Acc Chem Res 47, 2309-2318, doi:10.1021/ar500039w (2014).
30. Maeder, M. L. et al., Rapid “open-source” engineering of customized zinc-finger nucleases for highly efficient gene modification. Mol Cell 31, 294-301, doi:10.1016/j.molcel.2008.06.016 (2008).
31. Ramirez, C. L. et al. Unexpected failure rates for modular assembly of engineered zinc fingers. Nat Methods 5, 374-375, doi:10.1038/nmeth0508-374 (2008).
32. Wilcox, A. J., Choy, J., Bustamante, C. & Matouschek, A. Effect of protein structure on mitochondrial import. Proc Natl Acad Sci USA 102, 15435-15440, doi:10.1073/pnas.0507324102 (2005).
33. Li, J. Z. et al., Identification of a functional nuclear localization signal mediating nuclear import of the zinc finger transcription factor ZNF24. PLoS One 8, e79910, doi:10.1371/journal.pone.0079910 (2013).
34. Pandya, K. & Townes, T. M. Basic residues within the Kruppel zinc finger DNA binding domains are the critical nuclear localization determinants of EKLF/KLF-1. J Biol Chem 277, 16304-16312, doi:10.1074/jbc.M200866200 (2002).
35. Bhakta, M. S. et al. Highly active zinc-finger nucleases by extended modular assembly. Genome Res 23, 530-538, doi:10.1101/gr.143693.112 (2013).
36. Moore, M., Klug, A. & Choo, Y. Improved DNA binding specificity from polyzinc finger peptides by using strings of two-finger units. Proc Natl Acad Sci USA 98, 1437-1441, doi:10.1073/pnas.98.4.1437 (2001).
37. Papworth, M., Kolasinska, P. & Minczuk, M. Designer zinc-finger proteins and their applications. Gene 366, 27-38, doi:10.1016/j.gene.2005.09.011 (2006).
38. Kim, J. S. & Pabo, C. O. Getting a handhold on DNA: design of poly-zinc finger proteins with femtomolar dissociation constants. Proc Natl Acad Sci USA 95, 2812-2817, doi:10.1073/pnas.95.6.2812 (1998).
39. Nagaoka, M. et al., Multiconnection of identical zinc finger: implication for DNA binding affinity and unit modulation of the three zinc finger domain. Biochemistry 40, 2932-2941, doi:10.1021/bi001762+(2001).
40. Isalan, M., Choo, Y. & Klug, A. Synergy between adjacent zinc fingers in sequence-specific DNA recognition. Proc Natl Acad Sci USA 94, 5617-5621, doi:10.1073/pnas.94.11.5617 (1997).
41. Gill, J. S. et al., Pigmentary retinopathy, rod-cone dysfunction and sensorineural deafness associated with a rare mitochondrial tRNA(Lys) (m.8340G>A) gene variant. Br J Ophthalmol 101, 1298-1302, doi:10.1136/bjophthalmol-2017-310370 (2017).
42. Tarnopolsky, M. A., Sundaram, A. N. E., Provias, J., Brady, L. & Sadikovic, B. CPEO-Like mitochondrial myopathy associated with m.8340G>A mutation. Mitochondrion 46, 69-72, doi:10.1016/j.mito.2018.02.008 (2019).
43. Jeppesen, T. D. et al., A novel de novo mutation of the mitochondrial tRNAlys gene mt.8340G>a associated with pure myopathy. Neuromuscul Disord 24, 162-166, doi:10.1016/j.nmd.2013.08.004 (2014).
44. Richter, U. et al., RNA modification landscape of the human mitochondrial tRNA(Lys) regulates protein synthesis. Nat Commun 9, 3966, doi:10.1038/s41467-018-06471-z (2018).
45. Manickam, A. H., Michael, M. J. & Ramasamy, S. Mitochondrial genetics and therapeutic overview of Leber's hereditary optic neuropathy. Indian J Ophthalmol 65, 1087-1092, doi:10.4103/ijo.IJO_358_17 (2017).
46. Achilli, A. et al., Rare primary mitochondrial DNA mutations and probable synergistic variants in Leber's hereditary optic neuropathy. PLoS One 7, e42242, doi:10.1371/journal.pone.0042242 (2012).
47. Orkin, S. H. et al., ATA box transcription mutation in beta-thalassemia. Nucleic Acids Res 11, 4727-4734, doi:10.1093/nar/11.14.4727 (1983).
48. Gehrke, J. M. et al., An APOBEC3A-Cas9 base editor with minimized bystander and off-target activities. Nat Biotechnol 36, 977-982, doi:10.1038/nbt.4199 (2018).
49. Leach, K. M. et al., Characterization of the human beta-globin downstream promoter region. Nucleic Acids Res 31, 1292-1301, doi:10.1093/nar/gkg209 (2003).
50. Giardine, B. M. et al., Clinically relevant updates of the HbVar database of human hemoglobin variants and thalassemia mutations. Nucleic Acids Res 49, D1192-D1196, doi:10.1093/nar/gkaa959 (2021).
51. Gammage, P. A. et al., Genome editing in mitochondria corrects a pathogenic mtDNA mutation in vivo. Nat Med 24, 1691-1695, doi:10.1038/s41591-018-0165-9 (2018).
52. Vassalli, G., Bueler, H., Dudler, J., von Segesser, L. K. & Kappenberger, L. Adeno-associated virus (AAV) vectors achieve prolonged transgene expression in mouse myocardium and arteries in vivo: a comparative study with adenovirus vectors. Int J Cardiol 90, 229-238, doi:10.1016/s0167-5273(02)00554-5 (2003).
53. Ibraheim, R. et al., Self-inactivating, all-in-one AAV vectors for precision Cas9 genome editing via homology-directed repair in vivo. Nat Commun 12, 6267, doi:10.1038/s41467-021-26518-y (2021).
54. Li, Q. et al., In vivo PCSK9 gene editing using an all-in-one self-cleavage AAV-CRISPR system. Mol Ther Methods Clin Dev 20, 652-659, doi:10.1016/j.omtm.2021.02.005 (2021).
55. Li, A. et al., A Self-Deleting AAV-CRISPR System for In Vivo Genome Editing. Mol Ther Methods Clin Dev 12, 111-122, doi:10.1016/j.omtm.2018.11.009 (2019).
56. Silva-Pinheiro, P. et al., In vivo mitochondrial base editing via adeno-associated viral delivery to mouse post-mitotic tissue. Nat Commun 13, 750, doi:10.1038/s41467-022-28358-w (2022).
57. Zuris, J. A. et al., Cationic lipid-mediated delivery of proteins enables efficient protein-based genome editing in vitro and in vivo. Nat Biotechnol 33, 73-80, doi:10.1038/nbt.3081 (2015).
58. Rees, H. A. et al., Improving the DNA specificity and applicability of base editing through protein engineering and protein delivery. Nat Commun 8, 15790, doi:10.1038/ncomms15790 (2017).
59. Banskota, S. et al., Engineered virus-like particles for efficient in vivo delivery of therapeutic proteins. Cell 185, 250-265 e216, doi:10.1016/j.cell.2021.12.021 (2022).
60. Maeder, M. L., Thibodeau-Beganny, S., Sander, J. D., Voytas, D. F. & Joung, J. K. Oligomerized pool engineering (OPEN): an ‘open-source’ protocol for making customized zinc-finger arrays. Nat Protoc 4, 1471-1501, doi:10.1038/nprot.2009.98 (2009).
61. Sander, J. D. et al., Selection-free zinc-finger-nuclease engineering by context-dependent assembly (CoDA). Nat Methods 8, 67-69, doi:10.1038/nmeth.1542 (2011).
62. Clement, K. et al., CRISPResso2 provides accurate and rapid genome editing sequence analysis. Nat Biotechnol 37, 224-226, doi:10.1038/s41587-019-0032-3 (2019).
63. de Castro, E. et al., ScanProsite: detection of PROSITE signature matches and ProRule-associated functional and structural residues in proteins. Nucleic Acids Res 34, W362-365, doi:10.1093/nar/gkl124 (2006).
64. Crooks, G. E., Hon, G., Chandonia, J. M. & Brenner, S. E. WebLogo: a sequence logo generator. Genome Res 14, 1188-1190, doi:10.1101/gr.849004 (2004).
65. Cradick, T. J., Ambrosini, G., Iseli, C., Bucher, P. & McCaffrey, A. P. ZFN-site searches genomes for zinc finger nuclease target sites and off-target sites. BMC Bioinformatics 12, 152, doi:10.1186/1471-2105-12-152 (2011).
66. Isalan, M., Choo, Y. & Klug, A. Synergy between adjacent zinc fingers in sequence-specific DNA recognition. Proc Natl Acad Sci USA 94, 5617-5621, doi:10.1073/pnas.94.11.5617 (1997).
67. Mandell, J. G. & Barbas, C. F., 3^{rd}. Zinc Finger Tools: custom DNA-binding domains for transcription factors and nucleases. Nucleic Acids Res 34, W516-523, doi:10.1093/nar/gkl209 (2006).
68. Maeder, M. L. et al., Rapid “open-source” engineering of customized zinc-finger nucleases for highly efficient gene modification. Mol Cell 31, 294-301, doi:10.1016/j.molcel.2008.06.016 (2008).
69. Maeder, M. L., Thibodeau-Beganny, S., Sander, J. D., Voytas, D. F. & Joung, J. K. Oligomerized pool engineering (OPEN): an ‘open-source’ protocol for making customized zinc-finger arrays. Nat Protoc 4, 1471-1501, doi:10.1038/nprot.2009.98 (2009).
70. Sander, J. D. et al., Selection-free zinc-finger-nuclease engineering by context-dependent assembly (CoDA). Nat Methods 8, 67-69, doi:10.1038/nmeth.1542 (2011).
71. Shimizu, Y. et al., Adding fingers to an engineered zinc finger nuclease can reduce activity. Biochemistry 50, 5033-5041, doi:10.1021/bi200393g (2011).
72. Moore, M., Klug, A. & Choo, Y. Improved DNA binding specificity from polyzinc finger peptides by using strings of two-finger units. Proc Natl Acad Sci USA 98, 1437-1441, doi:10.1073/pnas.98.4.1437 (2001).
73. Bhakta, M. S. et al., Highly active zinc-finger nucleases by extended modular assembly. Genome Res 23, 530-538, doi:10.1101/gr.143693.112 (2013).
74. Nagaoka, M. et al., Multiconnection of identical zinc finger: implication for DNA binding affinity and unit modulation of the three zinc finger domain. Biochemistry 40, 2932-2941, doi:10.1021/bi001762+(2001).
75. Beerli, R. R., Segal, D. J., Dreier, B. & Barbas, C. F., 3rd. Toward controlling gene expression at will: specific regulation of the erbB-2/HER-2 promoter by using polydactyl zinc finger proteins constructed from modular building blocks. Proc Natl Acad Sci USA 95, 14628-14633, doi:10.1073/pnas.95.25.14628 (1998).

### Example 4. Correction of Disease-Causing Mutations Using ZF-DdCBEs

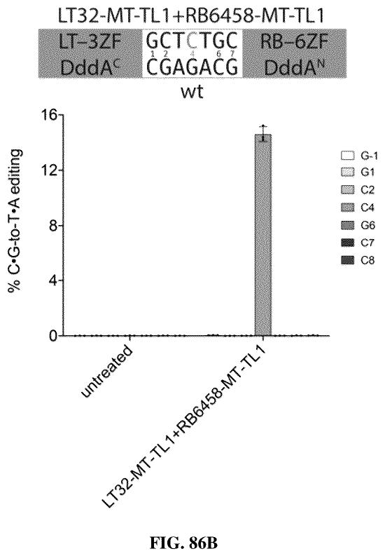

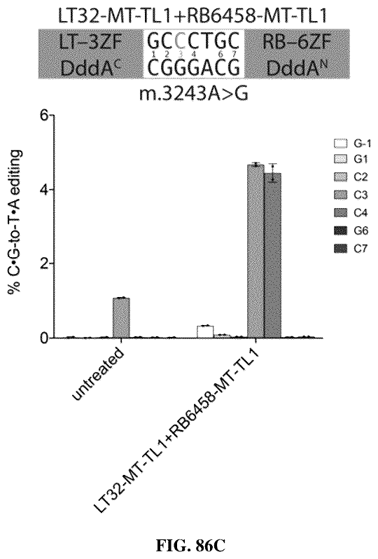

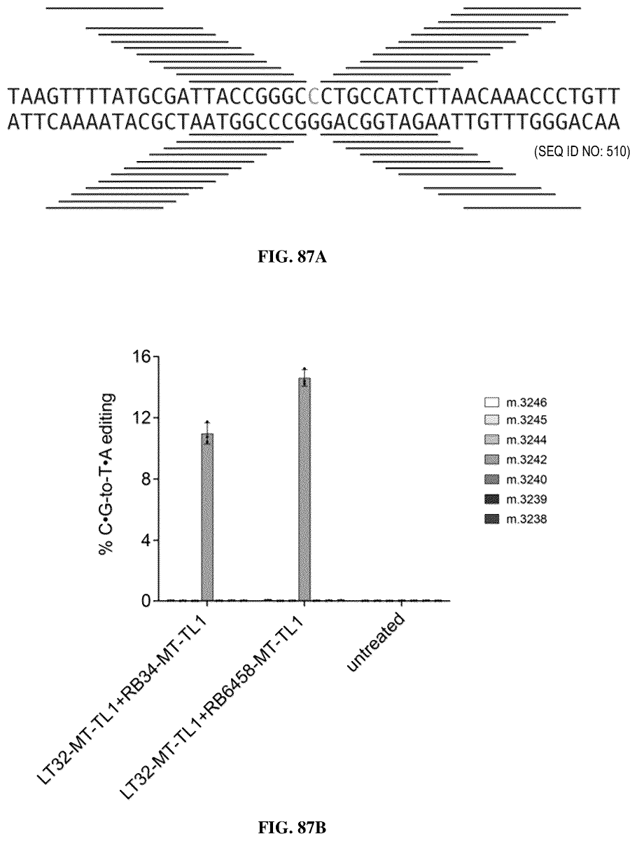

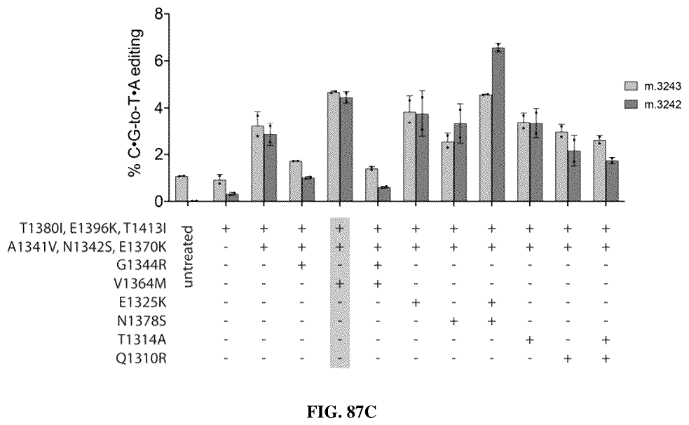

To demonstrate the ability of ZF-DdCBEs to correct disease-causing mutations, correcting the m.3243A>G mutation in the human MT-TL1 gene, which is associated with mitochondrial encephalomyopathy, lactic acidosis, and stroke-like episodes (MELAS), and is the most common human pathogenic mtDNA mutation^{1, 2}, was explored. This mutation impairs mt-tRNA^{Leu(UUR)} aminoacylation and post-transcriptional modification, disrupting mitochondrial translation³⁻⁵ (FIG. 86A). A panel of 22 left 3ZF ZF-DdCBEs was tested with 22 right 3ZF ZF-DdCBEs in both deaminase orientations, forming a total of 968 pairwise combinations in v7^{AGKS} architecture (FIG. 87A). Initially, HEK293T cells encoding wild-type MT-TL1, which lacks the m.3243A>G mutation, were used, and editing of the adjacent base at position m.3242 (CTC context) was screened for as a proxy for on-target editing activity. A single ZF-DdCBE pair able to efficiently install the desired edit was identified, yielding an editing efficiency of 12% (FIG. 87B). This pair was optimized by extending each 3ZF to 4ZF, 5ZF, or 6ZF in addition to testing alternative ZF DNA-recognition coding schemes. A pair was selected (MT-TL1•pB7-LT32/pB6N-RB6458) that showed a good balance between high on-target activity and low bystander or off-target editing. This final 3ZF/6ZF v7^{AGKS} ZF-DdCBE pair exhibited a 1.3-fold improvement relative to the unoptimized 3ZF/3ZF pair, installing the m.3242G>A mutation in HEK293T cells at an efficiency of 15% and with excellent specificity (FIG. 86B, FIG. 87B).

As a final step to develop ZF-DdCBEs able to correct the m.3243A>G mutation, it was investigated whether introducing mutations in DddA could enable efficient ZF-DdCBE editing at the disease-relevant CC context. PACE was recently used to evolve DddA variants that support improved TALE-based DdCBE activity at CC sequence contexts⁶. To assess if these variants improve ZF-DdCBEs, the effect of installing a series of these mutations (A1341V, N1342S, E1370K, G1344R, V1364M, E1325K, N1378S, Q1310R, and T1314A) into the best-performing ZF-DdCBE pair was tested on m.3243A>G correction efficiency in RN164 cybrid 143BTK⁻ cells homoplasmic for m.3243A>G (FIG. 87C). It was found that installing the additional mutations A1341V, N1342S, V1364M, and E1370K into DddA^{N} enabled correction of the m.3243A>G mutation (CCC context) at 5% editing efficiency (FIG. 86C). This was accompanied by 4% bystander editing of the adjacent nucleotide at m.3242, converting a G-U wobble base pair to an A-U Watson-Crick base pair in the tRNA D-arm, which preserves normal mt-tRNA^{Leu(UUR)} modification and is associated with non-MELAS symptoms^{3, 7, 8}. Collectively, these results demonstrate the potential for ZF-DdCBEs to make therapeutically relevant edits that correct mutations causing human mitochondrial genetic disease.

TABLE 31
ZF-DdCBEs targeting MT-TLI

| | Target | | | | |
|---|---|---|---|---|---|
| Name: | sequence: | ZF1: | ZF2: | ZF3: | ZF4: |
| LT32-MT-TL1 | GATTACCGG | RSDKLTE | SRGNLKS | TSGNLVR | — |
| | | (SEQ ID | (SEQ ID | (SEQ ID | |
| | | NO: 779) | NO: 802) | NO: 788) | |
| RB34-MT-TL1 | GTTAAGATG | RRDELNV | RKDNLKN | TSGSLVR | — |
| | | (SEQ ID | (SEQ ID | (SEQ ID | |
| | | NO: 767) | NO: 755) | NO: 800) | |
| RB6458-MT-TL1 | ACAGGGTTTGTTAAGATG | RRDELNV | RKDNLKN | TSGSLVR | TTGALTE |
| | | (SEQ ID | (SEQ ID | (SEQ ID | (SEQ ID |
| | | NO: 767) | NO: 755) | NO: 800) | NO: 784) |

| | | | Target | | |
|---|---|---|---|---|---|
| | | | DNA | DddA | |
| Name: | ZF5: | ZF6: | strand: | split: | Architecture: |
| LT32-MT-TL1 | — | — | LT | DddA^{C} | N-terminal |
| RB34-MT-TL1 | — | — | RB | DddA^{N} | N-terminal |
| RB6458-MT-TL1 | RSDHLSR | QSSVRNS | RB | DddA^{N} | N-terminal |

### REFERENCES FOR EXAMPLE 4

1. El-Hattab, A. W., Adesina, A. M., Jones, J., and Scaglia, F. MELAS syndrome: Clinical manifestations, pathogenesis, and treatment options. Mol Genet Metab 116, 4-12, doi:10.1016/j.ymgme.2015.06.004 (2015).
2. Majamaa, K. et al., Epidemiology of A3243G, the mutation for mitochondrial encephalomyopathy, lactic acidosis, and strokelike episodes: prevalence of the mutation in an adult population. Am J Hum Genet 63, 447-454, doi:10.1086/301959 (1998).
3. Kirino, Y., Goto, Y., Campos, Y., Arenas, J., and Suzuki, T. Specific correlation between the wobble modification deficiency in mutant tRNAs and the clinical features of a human mitochondrial disease. Proc Natl Acad Sci USA 102, 7127-7132, doi:10.1073/pnas.0500563102 (2005).
4. Hao, R., Yao, Y. N., Zheng, Y. G., Xu, M. G., and Wang, E. D. Reduction of mitochondrial tRNALeu(UUR) aminoacylation by some MELAS-associated mutations. FEBS Lett 578, 135-139, doi:10.1016/j.febslet.2004.11.004 (2004).
5. Borner, G. V. et al., Decreased aminoacylation of mutant tRNAs in MELAS but not in MERRF patients. Hum Mol Genet 9, 467-475, doi:10.1093/hmg/9.4.467 (2000).
6. Mok, B. Y. et al., CRISPR-free base editors with enhanced activity and expanded targeting scope in mitochondrial and nuclear DNA. Nat Biotechnol, doi:10.1038/s41587-022-01256-8 (2022).
7. Mimaki, M. et al., Different effects of novel mtDNA G3242A and G3244A base changes adjacent to a common A3243G mutation in patients with mitochondrial disorders. Mitochondrion 9, 115-122, doi:10.1016/j.mito.2009.01.005 (2009).
8. Wortmann, S. B. et al., Mitochondrial DNA m.3242G>A mutation, an under diagnosed cause of hypertrophic cardiomyopathy and renal tubular dysfunction? Eur J Med Genet 55, 552-556, doi:10.1016/j.ejmg.2012.06.002 (2012).

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

What is claimed is:

1. A zinc finger domain-containing protein comprising:
(i) one or more linker motifs, wherein each linker motif independently comprises the amino acid sequence of any one of SEQ ID NOs: 1-24;
(ii) one or more α-motifs, wherein each α-motif independently comprises the amino acid sequence of any one of SEQ ID NOs: 25-42 and 346; and
(iii) one or more β-motifs, wherein each β-motif independently comprises the amino acid sequence of any one of SEQ ID NOs: 43-138 and 336-345, or an amino acid sequence that is at least 90%, at least 95%, or at least 99% identical to the amino acid sequence of any one of SEQ ID NOs: 43-138 and 336-345.

2. The zinc finger domain-containing protein of claim 1, wherein the zinc finger domain-containing protein comprises the structure:
[first β-motif]-[first DNA recognition motif]-[first α-motif]-[first linker motif]-[second β-motif]-[second DNA recognition motif]-[second α-motif]-[second linker motif]-[third β-motif]-[third DNA recognition motif]-[third α-motif].

3. The zinc finger domain-containing protein of claim 2, wherein each of the first, second, and third β-motifs comprise the same amino acid sequence.

4. The zinc finger domain-containing protein of claim 2 or 3, wherein each of the first, second, and third α-motifs comprise the same amino acid sequence.

5. The zinc finger domain-containing protein of any one of claims 2-4, wherein each of the first and second linker motifs comprise the same amino acid sequence.

6. The zinc finger domain-containing protein of any one of claims 2-5, wherein each of the first, second, and third β-motifs comprise the same amino acid sequence, each of the first, second, and third α-motifs comprise the same amino acid sequence, and each of the first and second linker motifs comprise the same amino acid sequence.

7. The zinc finger domain-containing protein of claim 1, wherein the zinc finger domain-containing protein comprises the structure:
[first β-motif]-[first DNA recognition motif]-[first α-motif]-[first linker motif]-[second β-motif]-[second DNA recognition motif]-[second α-motif]-[second linker motif]-[third β-motif]-[third DNA recognition motif]-[third α-motif]-[third linker motif]-[fourth β-motif]-[fourth DNA recognition motif]-[fourth α-motif].

8. The zinc finger domain-containing protein of claim 7, wherein each of the first, second, third, and fourth β-motifs comprise the same amino acid sequence.

9. The zinc finger domain-containing protein of claim 7 or 8, wherein each of the first, second, third, and fourth α-motifs comprise the same amino acid sequence.

10. The zinc finger domain-containing protein of any one of claims 7-9, wherein each of the first, second, and third linker motifs comprise the same amino acid sequence.

11. The zinc finger domain-containing protein of any one of claims 7-10, wherein each of the first, second, third, and fourth β-motifs comprise the same amino acid sequence, each of the first, second, third, and fourth α-motifs comprise the same amino acid sequence, and each of the first, second, and third linker motifs comprise the same amino acid sequence.

12. The zinc finger domain-containing protein of claim 1, wherein the zinc finger domain-containing protein comprises the structure:
[first β-motif]-[first DNA recognition motif]-[first α-motif]-[first linker motif]-[second β-motif]-[second DNA recognition motif]-[second α-motif]-[second linker motif]-[third β-motif]-[third DNA recognition motif]-[third α-motif]-[third linker motif]-[fourth β-motif]-[fourth DNA recognition motif]-[fourth α-motif]-[fourth linker motif]-[fifth β-motif]-[fifth DNA recognition motif]-[fifth α-motif].

13. The zinc finger domain-containing protein of claim 12, wherein each of the first, second, third, fourth, and fifth β-motifs comprise the same amino acid sequence.

14. The zinc finger domain-containing protein of claim 12 or 13, wherein each of the first, second, third, fourth, and fifth α-motifs comprise the same amino acid sequence.

15. The zinc finger domain-containing protein of any one of claims 12-14, wherein each of the first, second, third, and fourth linker motifs comprise the same amino acid sequence.

16. The zinc finger domain-containing protein of any one of claims 12-15, wherein each of the first, second, third, fourth, and fifth β-motifs comprise the same amino acid sequence, each of the first, second, third, fourth, and fifth α-motifs comprise the same amino acid sequence, and each of the first, second, third, and fourth linker motifs comprise the same amino acid sequence.

17. The zinc finger domain-containing protein of claim 1, wherein the zinc finger domain-containing protein comprises the structure:
[first β-motif]-[first DNA recognition motif]-[first α-motif]-[first linker motif]-[second β-motif]-[second DNA recognition motif]-[second α-motif]-[second linker motif]-[third β-motif]-[third DNA recognition motif]-[third α-motif]-[third linker motif]-[fourth β-motif]-[fourth DNA recognition motif]-[fourth α-motif]-[fourth linker motif]-[fifth β-motif]-[fifth DNA recognition motif]-[fifth α-motif]-[fifth linker motif]-[sixth β-motif]-[sixth DNA recognition motif]-[sixth α-motif].

18. The zinc finger domain-containing protein of claim 17, wherein each of the first, second, third, fourth, fifth, and sixth β-motifs comprise the same amino acid sequence.

19. The zinc finger domain-containing protein of claim 17 or 18, wherein each of the first, second, third, fourth, fifth, and sixth α-motifs comprise the same amino acid sequence.

20. The zinc finger domain-containing protein of any one of claims 17-19, wherein each of the first, second, third, fourth, and fifth linker motifs comprise the same amino acid sequence.

21. The zinc finger domain-containing protein of any one of claims 17-20, wherein each of the first, second, third, fourth, fifth, and sixth β-motifs comprise the same amino acid sequence, each of the first, second, third, fourth, fifth, and sixth α-motifs comprise the same amino acid sequence, and each of the first, second, third, fourth, and fifth linker motifs comprise the same amino acid sequence.

22. The zinc finger domain-containing protein of any one of claims 1-21, wherein the zinc finger domain-containing protein comprises one or more linker motifs comprising the amino acid sequence of any one of TGEKP (SEQ ID NO: 1), SGEKP (SEQ ID NO: 13), SGERP (SEQ ID NO: 14), and SGDKP (SEQ ID NO: 17).

23. The zinc finger domain-containing protein of any one of claims 2-6, wherein the first and second linker motifs each comprise the amino acid sequence TGEKP (SEQ ID NO: 1), SGEKP (SEQ ID NO: 13), SGERP (SEQ ID NO: 14), or SGDKP (SEQ ID NO: 17).

24. The zinc finger domain-containing protein of any one of claims 7-11, wherein the first, second, and third linker motifs each comprise the amino acid sequence TGEKP (SEQ ID NO: 1), SGEKP (SEQ ID NO: 13), SGERP (SEQ ID NO: 14), or SGDKP (SEQ ID NO: 17).

25. The zinc finger domain-containing protein of any one of claims 12-16, wherein the first, second, third, and fourth linker motifs each comprise the amino acid sequence TGEKP (SEQ ID NO: 1), SGEKP (SEQ ID NO: 13), SGERP (SEQ ID NO: 14), or SGDKP (SEQ ID NO: 17).

26. The zinc finger domain-containing protein of any one of claims 17-21, wherein the first, second, third, fourth, and fifth linker motifs each comprise the amino acid sequence TGEKP (SEQ ID NO: 1), SGEKP (SEQ ID NO: 13), SGERP (SEQ ID NO: 14), or SGDKP (SEQ ID NO: 17).

27. The zinc finger domain-containing protein of any one of claims 1-26, wherein the zinc finger domain-containing protein comprises one or more α-motifs comprising the amino acid sequence of any one of HMRTH (SEQ ID NO: 33), HMKIH (SEQ ID NO: 34), HMKVH (SEQ ID NO: 35), HMKTH (SEQ ID NO: 36), and HIRTH (SEQ ID NO: 346).

28. The zinc finger domain-containing protein of any one of claims 2-6, wherein the first, second, and third α-motifs each comprise the amino acid sequence HMRTH (SEQ ID NO: 33), HMKIH (SEQ ID NO: 34), HMKVH (SEQ ID NO: 35), HMKTH (SEQ ID NO: 36), or HIRTH (SEQ ID NO: 346).

29. The zinc finger domain-containing protein of any one of claims 7-11, wherein the first, second, third, and fourth α-motifs each comprise the amino acid sequence HMRTH (SEQ ID NO: 33), HMKIH (SEQ ID NO: 34), HMKVH (SEQ ID NO: 35), HMKTH (SEQ ID NO: 36), or HIRTH (SEQ ID NO: 346).

30. The zinc finger domain-containing protein of any one of claims 12-16, wherein the first, second, third, fourth, and fifth α-motifs each comprise the amino acid sequence HMRTH (SEQ ID NO: 33), HMKIH (SEQ ID NO: 34), HMKVH (SEQ ID NO: 35), HMKTH (SEQ ID NO: 36), or HIRTH (SEQ ID NO: 346).

31. The zinc finger domain-containing protein of any one of claims 17-21, wherein the first, second, third, fourth, fifth, and sixth α-motifs each comprise the amino acid sequence HMRTH (SEQ ID NO: 33), HMKIH (SEQ ID NO: 34), HMKVH (SEQ ID NO: 35), HMKTH (SEQ ID NO: 36), or HIRTH (SEQ ID NO: 346).

32. The zinc finger domain-containing protein of any one of claims 1-31, wherein the zinc finger domain-containing protein comprises one or more β-motifs comprising the amino acid sequence of any one of YKCNECGKAFN (SEQ ID NO: 51), YKCNECGKSFN (SEQ ID NO: 54), YKCSECGKAFN (SEQ ID NO: 57), YKCEECGKAFN (SEQ ID NO: 63), FKCNECGKAFN (SEQ ID NO: 99), FKCNECGKSFN (SEQ ID NO: 102), FKCSECGKAFN (SEQ ID NO: 105), FKCEECGKAFS (SEQ ID NO: 109), FKCEECGKAFN (SEQ ID NO: 111), FKCEECGKSFN (SEQ ID NO: 114), YACPECGKSFS (SEQ ID NO: 337), and FACDICGRKFA (SEQ ID NO: 345).

33. The zinc finger domain-containing protein of any one of claims 2-6, wherein the first, second, and third β-motifs each comprise the amino acid sequence YKCNECGKAFN (SEQ ID NO: 51), YKCNECGKSFN (SEQ ID NO: 54), YKCSECGKAFN (SEQ ID NO: 57), YKCEECGKAFN (SEQ ID NO: 63), FKCNECGKAFN (SEQ ID NO: 99), FKCNECGKSFN (SEQ ID NO: 102), FKCSECGKAFN (SEQ ID NO: 105), FKCEECGKAFS (SEQ ID NO: 109), FKCEECGKAFN (SEQ ID NO: 111), FKCEECGKSFN (SEQ ID NO: 114), YACPECGKSFS (SEQ ID NO: 337), or FACDICGRKFA (SEQ ID NO: 345).

34. The zinc finger domain-containing protein of any one of claims 7-11, wherein the first, second, third, and fourth β-motifs each comprise the amino acid sequence YKCNECGKAFN (SEQ ID NO: 51), YKCNECGKSFN (SEQ ID NO: 54), YKCSECGKAFN (SEQ ID NO: 57), YKCEECGKAFN (SEQ ID NO: 63), FKCNECGKAFN (SEQ ID NO: 99), FKCNECGKSFN (SEQ ID NO: 102), FKCSECGKAFN (SEQ ID NO: 105), FKCEECGKAFS (SEQ ID NO: 109), FKCEECGKAFN (SEQ ID NO: 111), FKCEECGKSFN (SEQ ID NO: 114), YACPECGKSFS (SEQ ID NO: 337), or FACDICGRKFA (SEQ ID NO: 345).

35. The zinc finger domain-containing protein of any one of claims 12-16, wherein the first, second, third, fourth, and fifth β-motifs each comprise the amino acid sequence YKCNECGKAFN (SEQ ID NO: 51), YKCNECGKSFN (SEQ ID NO: 54), YKCSECGKAFN (SEQ ID NO: 57), YKCEECGKAFN (SEQ ID NO: 63), FKCNECGKAFN (SEQ ID NO: 99), FKCNECGKSFN (SEQ ID NO: 102), FKCSECGKAFN (SEQ ID NO: 105), FKCEECGKAFS (SEQ ID NO: 109), FKCEECGKAFN (SEQ ID NO: 111), FKCEECGKSFN (SEQ ID NO: 114), YACPECGKSFS (SEQ ID NO: 337), or FACDICGRKFA (SEQ ID NO: 345).

36. The zinc finger domain-containing protein of any one of claims 17-21, wherein each of the first, second, third, fourth, fifth, and sixth β-motifs each comprise the amino acid sequence YKCNECGKAFN (SEQ ID NO: 51), YKCNECGKSFN (SEQ ID NO: 54), YKCSECGKAFN (SEQ ID NO: 57), YKCEECGKAFN (SEQ ID NO: 63), FKCNECGKAFN (SEQ ID NO: 99), FKCNECGKSFN (SEQ ID NO: 102), FKCSECGKAFN (SEQ ID NO: 105), FKCEECGKAFS (SEQ ID NO: 109), FKCEECGKAFN (SEQ ID NO: 111), FKCEECGKSFN (SEQ ID NO: 114), YACPECGKSFS (SEQ ID NO: 337), or FACDICGRKFA (SEQ ID NO: 345).

37. The zinc finger domain-containing protein of any one of claims 1-36, wherein every β-motif comprises the amino acid sequence FACDICGRKFA (SEQ ID NO: 345), every α-motif comprises the amino acid sequence HIRTH (SEQ ID NO: 346), and every linker motif comprises the amino acid sequence TGEKP (SEQ ID NO: 1).

38. The zinc finger domain-containing protein of any one of claims 1-36, wherein every β-motif comprises the amino acid sequence YACPECGKSFS (SEQ ID NO: 337), every α-motif comprises the amino acid sequence HIRTH (SEQ ID NO: 346), and every linker motif comprises the amino acid sequence TGEKP (SEQ ID NO: 1).

39. The zinc finger domain-containing protein of any one of claims 1-36, wherein every β-motif comprises the amino acid sequence FKCEECGKAFN (SEQ ID NO: 111), every α-motif comprises the amino acid sequence HIRTH (SEQ ID NO: 346), and every linker motif comprises the amino acid sequence TGEKP (SEQ ID NO: 1).

40. The zinc finger domain-containing protein of any one of claims 1-36, wherein every β-motif comprises the amino acid sequence YKCEECGKAFN (SEQ ID NO: 63), every α-motif comprises the amino acid sequence HIRTH (SEQ ID NO: 346), and every linker motif comprises the amino acid sequence TGEKP (SEQ ID NO: 1).

41. A fusion protein comprising a zinc finger domain-containing protein of any one of claims 1-40 and an effector protein.

42. The fusion protein of claim 41, wherein the effector protein comprises nuclease activity, nickase activity, recombinase activity, deaminase activity, methyltransferase activity, methylase activity, acetylase activity, acetyltransferase activity, transcriptional activation activity, transcriptional repression activity, or polymerase activity.

43. The fusion protein of claim 41 or 42, wherein the effector protein is a nucleic acid editing protein.

44. The fusion protein of claim 43, wherein the nucleic acid editing protein comprises a deaminase domain.

45. The fusion protein of claim 44, wherein the deaminase domain is an adenosine deaminase domain.

46. The fusion protein of claim 44, wherein the deaminase domain is a cytidine deaminase domain.

47. The fusion protein of claim 46, wherein the cytidine deaminase domain is a double-stranded DNA cytidine deaminase (DddA) domain.

48. The fusion protein of any one of claims 41-47 further comprising one or more mitochondrial targeting sequences (MTS).

49. The fusion protein of any one of claims 41-48 further comprising one or more nuclear export sequences (NES).

50. The fusion protein of claim 49, wherein the NES is the NES of mitogen-activated protein kinase kinase (MAPKK).

51. The fusion protein of any one of claims 41-47 further comprising one or more nuclear localization sequences.

52. The fusion protein of any one of claims 41-51 further comprising one or more UGI domains.

53. The fusion protein of any one of claims 41-52, wherein the zinc finger domain-containing protein and the effector protein are joined by a linker.

54. The fusion protein of claim 53, wherein the linker is a glycine and serine-rich amino acid linker, optionally wherein the linker is about 13 amino acids in length.

55. The fusion protein of any one of claims 41-54, wherein the fusion protein comprises the structure NH₂-[MTS]-[FLAG tag]-[NES]-[NES]-[first zinc finger domain]-[second zinc finger domain]-[third zinc finger domain]-[optional fourth zinc finger domain]-[optional fifth zinc finger domain]-[optional sixth zinc finger domain]-[linker]-[split DddA]-[UGI]-COOH or NH₂-[MTS]-[FLAG tag]-[NES]-[NES]-[split DddA]-[linker]-[first zinc finger domain]-[second zinc finger domain]-[third zinc finger domain]-[optional fourth zinc finger domain]-[optional fifth zinc finger domain]-[optional sixth zinc finger domain]-[UGI]-COOH.

56. A double-stranded DNA cytidine deaminase (DddA) variant comprising a first fragment comprising an amino acid sequence that is at least 80%, at least 85%, at least 90%, at least 95%, or at least 99% identical to the amino acid sequence of SEQ ID NO: 139, and a second fragment comprising an amino acid sequence that is at least 80%, at least 85%, at least 90%, at least 95%, or at least 99% identical to the amino acid sequence of SEQ ID NO: 283, wherein the first fragment comprises one or more amino acid substitutions, truncations, or extensions relative to the amino acid sequence of SEQ ID NO: 139, and/or wherein the second fragment comprises one or more amino acid substitutions, truncations, or extensions relative to the amino acid sequence of SEQ ID NO: 283.

57. The DddA variant of claim 56, wherein the first fragment comprises one or more amino acid substitutions relative to the amino acid sequence of SEQ ID NO: 139.

58. The DddA variant of claim 56 or 57, wherein the first fragment comprises an amino acid sequence of any one of SEQ ID NOs: 140-252, or an amino acid sequence at least 80%, at least 85%, at least 90%, at least 95%, or at least 99% identical to the amino acid sequence of any one of SEQ ID NOs: 140-252.

59. The DddA variant of any one of claims 56-58, wherein the first fragment comprises an amino acid substitution at position N18.

60. The DddA variant of claim 59, wherein the amino acid substitution is an N18K substitution.

61. The DddA variant of any one of claims 56-60, wherein the first fragment comprises an amino acid substitution at position P25.

62. The DddA variant of claim 61, wherein the amino acid substitution is a P25K substitution.

63. The DddA variant of claim 61, wherein the amino acid substitution is a P25A substitution.

64. The DddA variant of any one of claims 56-63, wherein the first fragment comprises an N-terminal amino acid truncation.

65. The DddA variant of any one of claims 56-64, wherein the first fragment comprises an N-terminal amino acid truncation of 1-15 amino acids in length.

66. The DddA variant of claim 64 or 65, wherein the first fragment comprises the amino acid sequence of any one of SEQ ID NOs: 253-267.

67. The DddA variant of any one of claims 56-66, wherein the first fragment comprises a C-terminal amino acid truncation.

68. The DddA variant of any one of claims 56-67, wherein the first fragment comprises a C-terminal amino acid truncation of 1-15 amino acids in length.

69. The DddA variant of claim 67 or 68, wherein the first fragment comprises the amino acid sequence of any one of SEQ ID NOs: 268-282.

70. The DddA variant of any one of claims 56-69, wherein the second fragment comprises a C-terminal amino acid truncation.

71. The DddA variant of any one of claims 56-70, wherein the second fragment comprises a C-terminal amino acid truncation of 1-10 amino acids in length.

72. The DddA variant of claim 70 or 71, wherein the second fragment comprises a C-terminal amino acid truncation of 3 amino acids in length.

73. The DddA variant of claim 70 or 71, wherein the first fragment comprises the amino acid sequence of any one of SEQ ID NOs: 284-293.

74. The DddA variant of any one of claims 56-69, wherein the second fragment comprises a C-terminal amino acid extension.

75. The DddA variant of any one of claims 56-69, wherein the second fragment comprises a C-terminal amino acid extension of 1-15 amino acids in length.

76. The DddA variant of claim 74 or 75, wherein the first fragment comprises the amino acid sequence of any one of SEQ ID NOs: 294-308.

77. The DddA variant of any one of claims 56-76 further comprising a sequence of charged amino acid residues.

78. The DddA variant of claim 77, wherein the sequence of charged amino acid residues comprises the amino acid sequence of any one of SEQ ID NOs: 309-334.

79. The DddA variant of claim 77 or 78, wherein the sequence of charged amino acid residues weakens the binding affinity of the first fragment and the second fragment of the DddA variant to one another.

80. The DddA variant of any one of claims 56-79 further comprising a catalytically dead second DddA fragment fused to the first DddA fragment.

81. The DddA variant of claim 80, wherein the catalytically dead second DddA fragment comprises the amino acid sequence of SEQ ID NO: 335, or an amino acid sequence that is at least 80%, at least 85%, at least 90%, at least 95%, or at least 99% identical to the amino acid sequence of SEQ ID NO: 335.

82. The DddA variant of claim 56, wherein the first fragment comprises amino acid substitutions at positions N18 and P25, and wherein the second fragment comprises a C-terminal amino acid truncation of 3 amino acids in length.

83. The DddA variant of claim 82, wherein the first fragment comprises the amino acid substitutions N18K and P25A, and wherein the second fragment comprises a C-terminal amino acid truncation of 3 amino acids in length.

84. The DddA variant of claim 82, wherein the first fragment comprises the amino acid substitutions N18K and P25K, and wherein the second fragment comprises a C-terminal amino acid truncation of 3 amino acids in length.

85. A fusion protein comprising a programmable DNA binding protein (pDNAbp) and the first or second fragment of a DddA variant of any one of claims 56-84.

86. The fusion protein of claim 85, wherein the programmable DNA binding protein is a nucleic acid-programmable DNA binding protein (napDNAbp).

87. The fusion protein of claim 86, wherein the napDNAbp is a Cas9 domain.

88. The fusion protein of claim 86 or 87, wherein the napDNAbp is a nickase.

89. The fusion protein of claim 86 or 87, wherein the napDNAbp is a nuclease-inactive napDNAbp.

90. The fusion protein or claim 86, wherein the napDNAbp is selected from the group consisting of Cas9, Cas12e, Cas12d, Cas12a, Cas12b1, Cas13a, Cas12c, and Argonaute, and optionally has a nickase activity.

91. The fusion protein of claim 85, wherein the programmable DNA binding protein is a zinc finger protein or a TALE protein.

92. The fusion protein of any one of claims 85-91 further comprising one or more mitochondrial targeting sequences (MTS).

93. The fusion protein of any one of claims 85-92 further comprising one or more nuclear export sequences (NES).

94. The fusion protein of claim 93, wherein the NES is the NES of mitogen-activated protein kinase kinase (MAPKK).

95. The fusion protein of any one of claims 85-91 further comprising one or more nuclear localization sequences.

96. The fusion protein of any one of claims 85-95 further comprising one or more UGI domains.

97. The fusion protein of any one of claims 85-96, wherein the pDNAbp and the first or second fragment of the DddA variant are joined by a linker.

98. The fusion protein of claim 97, wherein the linker is a glycine and serine-rich amino acid linker, optionally wherein the linker is about 13 amino acids in length.

99. The fusion protein of any one of claims 85-98, wherein the fusion protein comprises the structure NH₂-[MTS]-[FLAG tag]-[NES]-[NES]-[first zinc finger domain]-[second zinc finger domain]-[third zinc finger domain]-[optional fourth zinc finger domain]-[optional fifth zinc finger domain]-[optional sixth zinc finger domain]-[linker]-[split DddA]-[UGI]-COOH or NH₂-[MTS]-[FLAG tag]-[NES]-[NES]-[split DddA]-[linker]-[first zinc finger domain]-[second zinc finger domain]-[third zinc finger domain]-[optional fourth zinc finger domain]-[optional fifth zinc finger domain]-[optional sixth zinc finger domain]-[UGI]-COOH.

100. A fusion protein comprising the zinc finger domain-containing protein of any one of claims 1-40 and the first or the second fragment of a DddA variant of any one of claims 56-84.

101. The fusion protein of claim 100 further comprising one or more mitochondrial targeting sequences (MTS).

102. The fusion protein of claim 100 or 101 further comprising one or more nuclear export sequences (NES).

103. The fusion protein of claim 102, wherein the NES is the NES of mitogen-activated protein kinase kinase (MAPKK).

104. The fusion protein of claim 100 further comprising one or more nuclear localization sequences.

105. The fusion protein of any one of claims 100-104 further comprising one or more UGI domains.

106. The fusion protein of any one of claims 100-105, wherein the zinc finger domain-containing protein and the first or the second fragment of the DddA variant are joined by a linker.

107. The fusion protein of claim 106, wherein the linker is a glycine and serine-rich amino acid linker, optionally wherein the linker is about 13 amino acids in length.

108. The fusion protein of any one of claims 100-107, wherein the fusion protein comprises the structure NH₂-[MTS]-[FLAG tag]-[NES]-[NES]-[first zinc finger domain]-[second zinc finger domain]-[third zinc finger domain]-[optional fourth zinc finger domain]-[optional fifth zinc finger domain]-[optional sixth zinc finger domain]-[linker]-[split DddA]-[UGI]-COOH or NH₂-[MTS]-[FLAG tag]-[NES]-[NES]-[split DddA]-[linker]-[first zinc finger domain]-[second zinc finger domain]-[third zinc finger domain]-[optional fourth zinc finger domain]-[optional fifth zinc finger domain]-[optional sixth zinc finger domain]-[UGI]-COOH.

109. A method for editing a target nucleic acid molecule comprising contacting the target nucleic acid molecule with the fusion protein of any one of claims 41-55 or 85-108.

110. The method of claim 109, wherein the target nucleic acid molecule comprises nuclear DNA.

111. The method of claim 109, wherein the target nucleic acid molecule comprises mitochondrial DNA.

112. The method of any one of claims 109-111, wherein the contacting is performed in vitro.

113. The method of any one of claims 109-111, wherein the contacting is performed in vivo.

114. The method of claim 113, wherein the contacting is performed in a subject.

115. The method of claim 114, wherein the subject has been diagnosed with a disease or disorder.

116. The method of any one of claims 109-115, wherein the target nucleic acid molecule comprises a genomic sequence associated with a disease or disorder.

117. The method of claim 116, wherein the target nucleic acid molecule comprises a point mutation associated with a disease or disorder.

118. The method of claim 117, wherein the point mutation comprises a T→C point mutation associated with a disease or disorder.

119. The method of claim 117, wherein the point mutation comprises an A→G point mutation associated with a disease or disorder.

120. The method of any one of claims 117-119, wherein the step of editing the target nucleic acid results in correction of the point mutation.

121. The method of any one of claims 109-120, wherein the target nucleic acid comprises MT-TK, Nd1, HBB, or MT-TL1.

122. The method of any one of claims 109-121, wherein the fusion protein comprises the architecture of any of the fusion proteins provided in Table 7, Table 8, and Table 31.

123. A polynucleotide encoding a zinc finger domain-containing protein of any one of claims 1-40, a DddA variant of any one of claims 56-84, or a fusion protein of any one of claims 41-55 or 85-108.

124. A vector comprising a polynucleotide of claim 123.

125. A cell comprising a zinc finger domain-containing protein of any one of claims 1-40, a DddA variant of any one of claims 56-84, a fusion protein of any one of claims 41-55 or 85-108, a polynucleotide of claim 123, or a vector of claim 124.

126. A kit comprising a zinc finger domain-containing protein of any one of claims 1-40, a DddA variant of any one of claims 56-84, a fusion protein of any one of claims 41-55 or 85-108, a polynucleotide of claim 123, a vector of claim 124, or a cell of claim 125.

127. A pharmaceutical composition comprising a zinc finger domain-containing protein of any one of claims 1-40, a DddA variant of any one of claims 56-84, a fusion protein of any one of claims 41-55 or 85-108, a polynucleotide of claim 123, or a vector of claim 124, and a pharmaceutically acceptable excipient.

128. An AAV comprising a fusion protein of any one of claims 41-55 or 85-108, a polynucleotide of claim 123, or a vector of claim 124.

129. A zinc finger domain-containing protein of any one of claims 1-40, a DddA variant of any one of claims 56-84, a fusion protein of any one of claims 41-55 or 85-108, a polynucleotide of claim 123, a vector of claim 124, a pharmaceutical composition of claim 127, or an AAV of claim 128 for use in medicine.

130. Use of a zinc finger domain-containing protein of any one of claims 1-40, a DddA variant of any one of claims 56-84, a fusion protein of any one of claims 41-55 or 85-108, a polynucleotide of claim 123, a vector of claim 124, a pharmaceutical composition of claim 127, or an AAV of claim 128 in the manufacture of a medicament for the treatment of a disease or disorder.


SEQUENCE LISTING (1087 sequences)

SEQ ID NO: 1 - protein, 5 aa, Synthetic construct
TGEKP

SEQ ID NO: 2 - protein, 5 aa, Synthetic construct
TGERP

SEQ ID NO: 3 - protein, 5 aa, Synthetic construct
TGKKP

SEQ ID NO: 4 - protein, 5 aa, Synthetic construct
TGKRP

SEQ ID NO: 5 - protein, 5 aa, Synthetic construct
TGDKP

SEQ ID NO: 6 - protein, 5 aa, Synthetic construct
TGDRP

SEQ ID NO: 7 - protein, 5 aa, Synthetic construct
TEEKP

SEQ ID NO: 8 - protein, 5 aa, Synthetic construct
TEERP

SEQ ID NO: 9 - protein, 5 aa, Synthetic construct
TEKKP

SEQ ID NO: 10 - protein, 5 aa, Synthetic construct
TEKRP

SEQ ID NO: 11 - protein, 5 aa, Synthetic construct
TEDKP

SEQ ID NO: 12 - protein, 5 aa, Synthetic construct
TEDRP

SEQ ID NO: 13 - protein, 5 aa, Synthetic construct
SGEKP

SEQ ID NO: 14 - protein, 5 aa, Synthetic construct
SGERP

SEQ ID NO: 15 - protein, 5 aa, Synthetic construct
SGKKP

SEQ ID NO: 16 - protein, 5 aa, Synthetic construct
SGKRP

SEQ ID NO: 17 - protein, 5 aa, Synthetic construct
SGDKP

SEQ ID NO: 18 - protein, 5 aa, Synthetic construct
SGDRP

SEQ ID NO: 19 - protein, 5 aa, Synthetic construct
SEEKP

SEQ ID NO: 20 - protein, 5 aa, Synthetic construct
SEERP

SEQ ID NO: 21 - protein, 5 aa, Synthetic construct
SEKKP

SEQ ID NO: 22 - protein, 5 aa, Synthetic construct
SEKRP

SEQ ID NO: 23 - protein, 5 aa, Synthetic construct
SEDKP

SEQ ID NO: 24 - protein, 5 aa, Synthetic construct
SEDRP

SEQ ID NO: 25 - protein, 5 aa, Synthetic construct
HQRIH

SEQ ID NO: 26 - protein, 5 aa, Synthetic construct
HQRVH

SEQ ID NO: 27 - protein, 5 aa, Synthetic construct
HQRTH

SEQ ID NO: 28 - protein, 5 aa, Synthetic construct
HQKIH

SEQ ID NO: 29 - protein, 5 aa, Synthetic construct
HQKVH

SEQ ID NO: 30 - protein, 5 aa, Synthetic construct
HQKTH

SEQ ID NO: 31 - protein, 5 aa, Synthetic construct
HMRIH

SEQ ID NO: 32 - protein, 5 aa, Synthetic construct
HMRVH

SEQ ID NO: 33 - protein, 5 aa, Synthetic construct
HMRTH

SEQ ID NO: 34 - protein, 5 aa, Synthetic construct
HMKIH

SEQ ID NO: 35 - protein, 5 aa, Synthetic construct
HMKVH

SEQ ID NO: 36 - protein, 5 aa, Synthetic construct
HMKTH

SEQ ID NO: 37 - protein, 5 aa, Synthetic construct
HKRIH

SEQ ID NO: 38 - protein, 5 aa, Synthetic construct
HKRVH

SEQ ID NO: 39 - protein, 5 aa, Synthetic construct
HKRTH

SEQ ID NO: 40 - protein, 5 aa, Synthetic construct
HKKIH

SEQ ID NO: 41 - protein, 5 aa, Synthetic construct
HKKVH

SEQ ID NO: 42 - protein, 5 aa, Synthetic construct
HKKTH

SEQ ID NO: 43 - protein, 11 aa, Synthetic construct
YKCKECGKAFS

SEQ ID NO: 44 - protein, 11 aa, Synthetic construct
YKCKECGKAFR

SEQ ID NO: 45 - protein, 11 aa, Synthetic construct
YKCKECGKAFN

SEQ ID NO: 46 - protein, 11 aa, Synthetic construct
YKCKECGKSFS

SEQ ID NO: 47 - protein, 11 aa, Synthetic construct
YKCKECGKSFR

SEQ ID NO: 48 - protein, 11 aa, Synthetic construct
YKCKECGKSFN

SEQ ID NO: 49 - protein, 11 aa, Synthetic construct
YKCNECGKAFS

SEQ ID NO: 50 - protein, 11 aa, Synthetic construct
YKCNECGKAFR

SEQ ID NO: 51 - protein, 11 aa, Synthetic construct
YKCNECGKAFN

SEQ ID NO: 52 - protein, 11 aa, Synthetic construct
YKCNECGKSFS

SEQ ID NO: 53 - protein, 11 aa, Synthetic construct
YKCNECGKSFR

SEQ ID NO: 54 - protein, 11 aa, Synthetic construct
YKCNECGKSFN

SEQ ID NO: 55 - protein, 11 aa, Synthetic construct
YKCSECGKAFS

SEQ ID NO: 56 - protein, 11 aa, Synthetic construct
YKCSECGKAFR

SEQ ID NO: 57 - protein, 11 aa, Synthetic construct
YKCSECGKAFN

SEQ ID NO: 58 - protein, 11 aa, Synthetic construct
YKCSECGKSFS

SEQ ID NO: 59 - protein, 11 aa, Synthetic construct
YKCSECGKSFR

SEQ ID NO: 60 - protein, 11 aa, Synthetic construct
YKCSECGKSFN

SEQ ID NO: 61 - protein, 11 aa, Synthetic construct
YKCEECGKAFS

SEQ ID NO: 62 - protein, 11 aa, Synthetic construct
YKCEECGKAFR

SEQ ID NO: 63 - protein, 11 aa, Synthetic construct
YKCEECGKAFN

SEQ ID NO: 64 - protein, 11 aa, Synthetic construct
YKCEECGKSFS

SEQ ID NO: 65 - protein, 11 aa, Synthetic construct
YKCEECGKSFR

SEQ ID NO: 66 - protein, 11 aa, Synthetic construct
YKCEECGKSFN

SEQ ID NO: 67 - protein, 11 aa, Synthetic construct
YECKECGKAFS

SEQ ID NO: 68 - protein, 11 aa, Synthetic construct
YECKECGKAFR

SEQ ID NO: 69 - protein, 11 aa, Synthetic construct
YECKECGKAFN

SEQ ID NO: 70 - protein, 11 aa, Synthetic construct
YECKECGKSFS

SEQ ID NO: 71 - protein, 11 aa, Synthetic construct
YECKECGKSFR

SEQ ID NO: 72 - protein, 11 aa, Synthetic construct
YECKECGKSFN

SEQ ID NO: 73 - protein, 11 aa, Synthetic construct
YECNECGKAFS

SEQ ID NO: 74 - protein, 11 aa, Synthetic construct
YECNECGKAFR

SEQ ID NO: 75 - protein, 11 aa, Synthetic construct
YECNECGKAFN

SEQ ID NO: 76 - protein, 11 aa, Synthetic construct
YECNECGKSFS

SEQ ID NO: 77 - protein, 11 aa, Synthetic construct
YECNECGKSFR

SEQ ID NO: 78 - protein, 11 aa, Synthetic construct
YECNECGKSFN

SEQ ID NO: 79 - protein, 11 aa, Synthetic construct
YECSECGKAFS

SEQ ID NO: 80 - protein, 11 aa, Synthetic construct
YECSECGKAFR

SEQ ID NO: 81 - protein, 11 aa, Synthetic construct
YECSECGKAFN

SEQ ID NO: 82 - protein, 11 aa, Synthetic construct
YECSECGKSFS

SEQ ID NO: 83 - protein, 11 aa, Synthetic construct
YECSECGKSFR

SEQ ID NO: 84 - protein, 11 aa, Synthetic construct
YECSECGKSFN

SEQ ID NO: 85 - protein, 11 aa, Synthetic construct
YECEECGKAFS

SEQ ID NO: 86 - protein, 11 aa, Synthetic construct
YECEECGKAFR

SEQ ID NO: 87 - protein, 11 aa, Synthetic construct
YECEECGKAFN

SEQ ID NO: 88 - protein, 11 aa, Synthetic construct
YECEECGKSFS

SEQ ID NO: 89 - protein, 11 aa, Synthetic construct
YECEECGKSFR

SEQ ID NO: 90 - protein, 11 aa, Synthetic construct
YECEECGKSFN

SEQ ID NO: 91 - protein, 11 aa, Synthetic construct
FKCKECGKAFS

SEQ ID NO: 92 - protein, 11 aa, Synthetic construct
FKCKECGKAFR

SEQ ID NO: 93 - protein, 11 aa, Synthetic construct
FKCKECGKAFN

SEQ ID NO: 94 - protein, 11 aa, Synthetic construct
FKCKECGKSFS

SEQ ID NO: 95 - protein, 11 aa, Synthetic construct
FKCKECGKSFR

SEQ ID NO: 96 - protein, 11 aa, Synthetic construct
FKCKECGKSFN

SEQ ID NO: 97 - protein, 11 aa, Synthetic construct
FKCNECGKAFS

SEQ ID NO: 98 - protein, 11 aa, Synthetic construct
FKCNECGKAFR

SEQ ID NO: 99 - protein, 11 aa, Synthetic construct
FKCNECGKAFN

SEQ ID NO: 100 - protein, 11 aa, Synthetic construct
FKCNECGKSFS

SEQ ID NO: 101 - protein, 11 aa, Synthetic construct
FKCNECGKSFR

SEQ ID NO: 102 - protein, 11 aa, Synthetic construct
FKCNECGKSFN

SEQ ID NO: 103 - protein, 11 aa, Synthetic construct
FKCSECGKAFS

SEQ ID NO: 104 - protein, 11 aa, Synthetic construct
FKCSECGKAFR

SEQ ID NO: 105 - protein, 11 aa, Synthetic construct
FKCSECGKAFN

SEQ ID NO: 106 - protein, 11 aa, Synthetic construct
FKCSECGKSFS

SEQ ID NO: 107 - protein, 11 aa, Synthetic construct
FKCSECGKSFR

SEQ ID NO: 108 - protein, 11 aa, Synthetic construct
FKCSECGKSFN

SEQ ID NO: 109 - protein, 11 aa, Synthetic construct
FKCEECGKAFS

SEQ ID NO: 110 - protein, 11 aa, Synthetic construct
FKCEECGKAFR

SEQ ID NO: 111 - protein, 11 aa, Synthetic construct
FKCEECGKAFN

SEQ ID NO: 112 - protein, 11 aa, Synthetic construct
FKCEECGKSFS

SEQ ID NO: 113 - protein, 11 aa, Synthetic construct
FKCEECGKSFR

SEQ ID NO: 114 - protein, 11 aa, Synthetic construct
FKCEECGKSFN

SEQ ID NO: 115 - protein, 11 aa, Synthetic construct
FECKECGKAFS

SEQ ID NO: 116 - protein, 11 aa, Synthetic construct
FECKECGKAFR

SEQ ID NO: 117 - protein, 11 aa, Synthetic construct
FECKECGKAFN

SEQ ID NO: 118 - protein, 11 aa, Synthetic construct
FECKECGKSFS

SEQ ID NO: 119 - protein, 11 aa, Synthetic construct
FECKECGKSFR

SEQ ID NO: 120 - protein, 11 aa, Synthetic construct
FECKECGKSFN

SEQ ID NO: 121 - protein, 11 aa, Synthetic construct
FECNECGKAFS

SEQ ID NO: 122 - protein, 11 aa, Synthetic construct
FECNECGKAFR

SEQ ID NO: 123 - protein, 11 aa, Synthetic construct
FECNECGKAFN

SEQ ID NO: 124 - protein, 11 aa, Synthetic construct
FECNECGKSFS

SEQ ID NO: 125 - protein, 11 aa, Synthetic construct
FECNECGKSFR

SEQ ID NO: 126 - protein, 11 aa, Synthetic construct
FECNECGKSFN

SEQ ID NO: 127 - protein, 11 aa, Synthetic construct
FECSECGKAFS

SEQ ID NO: 128 - protein, 11 aa, Synthetic construct
FECSECGKAFR

SEQ ID NO: 129 - protein, 11 aa, Synthetic construct
FECSECGKAFN

SEQ ID NO: 130 - protein, 11 aa, Synthetic construct
FECSECGKSFS

SEQ ID NO: 131 - protein, 11 aa, Synthetic construct
FECSECGKSFR

SEQ ID NO: 132 - protein, 11 aa, Synthetic construct
FECSECGKSFN

SEQ ID NO: 133 - protein, 11 aa, Synthetic construct
FECEECGKAFS

SEQ ID NO: 134 - protein, 11 aa, Synthetic construct
FECEECGKAFR

SEQ ID NO: 135 - protein, 11 aa, Synthetic construct
FECEECGKAFN

SEQ ID NO: 136 - protein, 11 aa, Synthetic construct
FECEECGKSFS

SEQ ID NO: 137 - protein, 11 aa, Synthetic construct
FECEECGKSFR

SEQ ID NO: 138 - protein, 11 aa, Synthetic construct
FECEECGKSFN

SEQ ID NO: 139 - protein, 30 aa, Synthetic construct
AIPVKRGATGETKVFTGNSNSPKSPTKGGC

SEQ ID NO: 140 - protein, 30 aa, Synthetic construct
AAPVKRGATGETKVFTGNSNSPKSPTKGGC

SEQ ID NO: 141 - protein, 30 aa, Synthetic construct
AIAVKRGATGETKVFTGNSNSPKSPTKGGC

SEQ ID NO: 142 - protein, 30 aa, Synthetic construct
AIPAKRGATGETKVFTGNSNSPKSPTKGGC

SEQ ID NO: 143 - protein, 30 aa, Synthetic construct
AIPVARGATGETKVFTGNSNSPKSPTKGGC

SEQ ID NO: 144 - protein, 30 aa, Synthetic construct
AIPVKAGATGETKVFTGNSNSPKSPTKGGC

SEQ ID NO: 145 - protein, 30 aa, Synthetic construct
AIPVKRAATGETKVFTGNSNSPKSPTKGGC

SEQ ID NO: 146 - protein, 30 aa, Synthetic construct
AIPVKRGAAGETKVFTGNSNSPKSPTKGGC

SEQ ID NO: 147 - protein, 30 aa, Synthetic construct
AIPVKRGATAETKVFTGNSNSPKSPTKGGC

SEQ ID NO: 148 - protein, 30 aa, Synthetic construct
AIPVKRGATGATKVFTGNSNSPKSPTKGGC

SEQ ID NO: 149 - protein, 30 aa, Synthetic construct
AIPVKRGATGEAKVFTGNSNSPKSPTKGGC

SEQ ID NO: 150 - protein, 30 aa, Synthetic construct
AIPVKRGATGETAVFTGNSNSPKSPTKGGC

SEQ ID NO: 151 - protein, 30 aa, Synthetic construct
AIPVKRGATGETKAFTGNSNSPKSPTKGGC

SEQ ID NO: 152 - protein, 30 aa, Synthetic construct
AIPVKRGATGETKVATGNSNSPKSPTKGGC

SEQ ID NO: 153 - protein, 30 aa, Synthetic construct
AIPVKRGATGETKVFAGNSNSPKSPTKGGC

SEQ ID NO: 154 - protein, 30 aa, Synthetic construct
AIPVKRGATGETKVFTANSNSPKSPTKGGC

SEQ ID NO: 155 - protein, 30 aa, Synthetic construct
AIPVKRGATGETKVFTGASNSPKSPTKGGC

SEQ ID NO: 156 - protein, 30 aa, Synthetic construct
AIPVKRGATGETKVFTGNANSPKSPTKGGC

SEQ ID NO: 157 - protein, 30 aa, Synthetic construct
AIPVKRGATGETKVFTGNSASPKSPTKGGC

SEQ ID NO: 158 - protein, 30 aa, Synthetic construct
AIPVKRGATGETKVFTGNSNAPKSPTKGGC

SEQ ID NO: 159 - protein, 30 aa, Synthetic construct
AIPVKRGATGETKVFTGNSNSAKSPTKGGC

SEQ ID NO: 160 - protein, 30 aa, Synthetic construct
AIPVKRGATGETKVFTGNSNSPASPTKGGC

SEQ ID NO: 161 - protein, 30 aa, Synthetic construct
AIPVKRGATGETKVFTGNSNSPKAPTKGGC

SEQ ID NO: 162 - protein, 30 aa, Synthetic construct
AIPVKRGATGETKVFTGNSNSPKSATKGGC

SEQ ID NO: 163 - protein, 30 aa, Synthetic construct
AIPVKRGATGETKVFTGNSNSPKSPAKGGC

SEQ ID NO: 164 - protein, 30 aa, Synthetic construct
AIPVKRGATGETKVFTGNSNSPKSPTAGGC

SEQ ID NO: 165 - protein, 30 aa, Synthetic construct
AIPVKRGATGETKVFTGNSNSPKSPTKAGC

SEQ ID NO: 166 - protein, 30 aa, Synthetic construct
AIPVKRGATGETKVFTGNSNSPKSPTKGAC

SEQ ID NO: 167 - protein, 30 aa, Synthetic construct
AIPVKRGATGETKVFTGNSNSPKSPTKGGA

SEQ ID NO: 168 - protein, 30 aa, Synthetic construct
KIPVKRGATGETKVFTGNSNSPKSPTKGGC

SEQ ID NO: 169 - protein, 30 aa, Synthetic construct
AKPVKRGATGETKVFTGNSNSPKSPTKGGC

SEQ ID NO: 170 - protein, 30 aa, Synthetic construct
AIKVKRGATGETKVFTGNSNSPKSPTKGGC

SEQ ID NO: 171 - protein, 30 aa, Synthetic construct
AIPKKRGATGETKVFTGNSNSPKSPTKGGC

SEQ ID NO: 172 - protein, 30 aa, Synthetic construct
AIPVKKGATGETKVFTGNSNSPKSPTKGGC

SEQ ID NO: 173 - protein, 30 aa, Synthetic construct
AIPVKRKATGETKVFTGNSNSPKSPTKGGC

SEQ ID NO: 174 - protein, 30 aa, Synthetic construct
AIPVKRGKTGETKVFTGNSNSPKSPTKGGC

SEQ ID NO: 175 - protein, 30 aa, Synthetic construct
AIPVKRGAKGETKVFTGNSNSPKSPTKGGC

SEQ ID NO: 176 - protein, 30 aa, Synthetic construct
AIPVKRGATKETKVFTGNSNSPKSPTKGGC

SEQ ID NO: 177 - protein, 30 aa, Synthetic construct
AIPVKRGATGKTKVFTGNSNSPKSPTKGGC

SEQ ID NO: 178 - protein, 30 aa, Synthetic construct
AIPVKRGATGEKKVFTGNSNSPKSPTKGGC

SEQ ID NO: 179 - protein, 30 aa, Synthetic construct
AIPVKRGATGETKKFTGNSNSPKSPTKGGC

SEQ ID NO: 180 - protein, 30 aa, Synthetic construct
AIPVKRGATGETKVKTGNSNSPKSPTKGGC

SEQ ID NO: 181 - protein, 30 aa, Synthetic construct
AIPVKRGATGETKVFKGNSNSPKSPTKGGC

SEQ ID NO: 182 - protein, 30 aa, Synthetic construct
AIPVKRGATGETKVFTKNSNSPKSPTKGGC

SEQ ID NO: 183 - protein, 30 aa, Synthetic construct
AIPVKRGATGETKVFTGKSNSPKSPTKGGC

SEQ ID NO: 184 - protein, 30 aa, Synthetic construct
AIPVKRGATGETKVFTGNKNSPKSPTKGGC

SEQ ID NO: 185 - protein, 30 aa, Synthetic construct
AIPVKRGATGETKVFTGNSKSPKSPTKGGC

SEQ ID NO: 186 - protein, 30 aa, Synthetic construct
AIPVKRGATGETKVFTGNSNKPKSPTKGGC

SEQ ID NO: 187 - protein, 30 aa, Synthetic construct
AIPVKRGATGETKVFTGNSNSKKSPTKGGC

SEQ ID NO: 188 - protein, 30 aa, Synthetic construct
AIPVKRGATGETKVFTGNSNSPKKPTKGGC

SEQ ID NO: 189 - protein, 30 aa, Synthetic construct
AIPVKRGATGETKVFTGNSNSPKSKTKGGC

SEQ ID NO: 190 - protein, 30 aa, Synthetic construct
AIPVKRGATGETKVFTGNSNSPKSPKKGGC

SEQ ID NO: 191 - protein, 30 aa, Synthetic construct
AIPVKRGATGETKVFTGNSNSPKSPTKKGC

SEQ ID NO: 192 - protein, 30 aa, Synthetic construct
AIPVKRGATGETKVFTGNSNSPKSPTKGKC

SEQ ID NO: 193 - protein, 30 aa, Synthetic construct
AIPVKRGATGETKVFTGNSNSPKSPTKGGK

SEQ ID NO: 194 - protein, 30 aa, Synthetic construct
DIPVKRGATGETKVFTGNSNSPKSPTKGGC

SEQ ID NO: 195 - protein, 30 aa, Synthetic construct
ADPVKRGATGETKVFTGNSNSPKSPTKGGC

SEQ ID NO: 196 - protein, 30 aa, Synthetic construct
AIDVKRGATGETKVFTGNSNSPKSPTKGGC

SEQ ID NO: 197 - protein, 30 aa, Synthetic construct
AIPDKRGATGETKVFTGNSNSPKSPTKGGC

SEQ ID NO: 198 - protein, 30 aa, Synthetic construct
AIPVDRGATGETKVFTGNSNSPKSPTKGGC

SEQ ID NO: 199 - protein, 30 aa, Synthetic construct
AIPVKDGATGETKVFTGNSNSPKSPTKGGC

SEQ ID NO: 200 - protein, 30 aa, Synthetic construct
AIPVKRDATGETKVFTGNSNSPKSPTKGGC

SEQ ID NO: 201 - protein, 30 aa, Synthetic construct
AIPVKRGDTGETKVFTGNSNSPKSPTKGGC

SEQ ID NO: 202 - protein, 30 aa, Synthetic construct
AIPVKRGADGETKVFTGNSNSPKSPTKGGC

SEQ ID NO: 203 - protein, 30 aa, Synthetic construct
AIPVKRGATDETKVFTGNSNSPKSPTKGGC

SEQ ID NO: 204 - protein, 30 aa, Synthetic construct
AIPVKRGATGDTKVFTGNSNSPKSPTKGGC

SEQ ID NO: 205 - protein, 30 aa, Synthetic construct
AIPVKRGATGEDKVFTGNSNSPKSPTKGGC

SEQ ID NO: 206 - protein, 30 aa, Synthetic construct
AIPVKRGATGETDVFTGNSNSPKSPTKGGC

SEQ ID NO: 207 - protein, 30 aa, Synthetic construct
AIPVKRGATGETKDFTGNSNSPKSPTKGGC

SEQ ID NO: 208 - protein, 30 aa, Synthetic construct
AIPVKRGATGETKVDTGNSNSPKSPTKGGC

SEQ ID NO: 209 - protein, 30 aa, Synthetic construct
AIPVKRGATGETKVFDGNSNSPKSPTKGGC

SEQ ID NO: 210 - protein, 30 aa, Synthetic construct
AIPVKRGATGETKVFTDNSNSPKSPTKGGC

SEQ ID NO: 211 - protein, 30 aa, Synthetic construct
AIPVKRGATGETKVFTGDSNSPKSPTKGGC

SEQ ID NO: 212 - protein, 30 aa, Synthetic construct
AIPVKRGATGETKVFTGNDNSPKSPTKGGC

SEQ ID NO: 213 - protein, 30 aa, Synthetic construct
AIPVKRGATGETKVFTGNSDSPKSPTKGGC

SEQ ID NO: 214 - protein, 30 aa, Synthetic construct
AIPVKRGATGETKVFTGNSNDPKSPTKGGC

SEQ ID NO: 215 - protein, 30 aa, Synthetic construct
AIPVKRGATGETKVFTGNSNSDKSPTKGGC

SEQ ID NO: 216 - protein, 30 aa, Synthetic construct
AIPVKRGATGETKVFTGNSNSPDSPTKGGC

SEQ ID NO: 217 - protein, 30 aa, Synthetic construct
AIPVKRGATGETKVFTGNSNSPKDPTKGGC

SEQ ID NO: 218 - protein, 30 aa, Synthetic construct
AIPVKRGATGETKVFTGNSNSPKSDTKGGC

SEQ ID NO: 219 - protein, 30 aa, Synthetic construct
AIPVKRGATGETKVFTGNSNSPKSPDKGGC

SEQ ID NO: 220 - protein, 30 aa, Synthetic construct
AIPVKRGATGETKVFTGNSNSPKSPTDGGC

SEQ ID NO: 221 - protein, 30 aa, Synthetic construct
AIPVKRGATGETKVFTGNSNSPKSPTKDGC

SEQ ID NO: 222 - protein, 30 aa, Synthetic construct
AIPVKRGATGETKVFTGNSNSPKSPTKGDC

SEQ ID NO: 223 - protein, 30 aa, Synthetic construct
AIPVKRGATGETKVFTGNSNSPKSPTKGGD

SEQ ID NO: 224 - protein, 30 aa, Synthetic construct
EIPVKRGATGETKVFTGNSNSPKSPTKGGC

SEQ ID NO: 225 - protein, 30 aa, Synthetic construct
AEPVKRGATGETKVFTGNSNSPKSPTKGGC

SEQ ID NO: 226 - protein, 30 aa, Synthetic construct
AIEVKRGATGETKVFTGNSNSPKSPTKGGC

SEQ ID NO: 227 - protein, 30 aa, Synthetic construct
AIPEKRGATGETKVFTGNSNSPKSPTKGGC

SEQ ID NO: 228 - protein, 30 aa, Synthetic construct
AIPVERGATGETKVFTGNSNSPKSPTKGGC

SEQ ID NO: 229 - protein, 30 aa, Synthetic construct
AIPVKEGATGETKVFTGNSNSPKSPTKGGC

SEQ ID NO: 230 - protein, 30 aa, Synthetic construct
AIPVKREATGETKVFTGNSNSPKSPTKGGC

SEQ ID NO: 231 - protein, 30 aa, Synthetic construct
AIPVKRGETGETKVFTGNSNSPKSPTKGGC

SEQ ID NO: 232 - protein, 30 aa, Synthetic construct
AIPVKRGAEGETKVFTGNSNSPKSPTKGGC

SEQ ID NO: 233 - protein, 30 aa, Synthetic construct
AIPVKRGATEETKVFTGNSNSPKSPTKGGC

SEQ ID NO: 234 - protein, 30 aa, Synthetic construct
AIPVKRGATGEEKVFTGNSNSPKSPTKGGC

SEQ ID NO: 235 - protein, 30 aa, Synthetic construct
AIPVKRGATGETEVFTGNSNSPKSPTKGGC

SEQ ID NO: 236 - protein, 30 aa, Synthetic construct
AIPVKRGATGETKEFTGNSNSPKSPTKGGC

SEQ ID NO: 237 - protein, 30 aa, Synthetic construct
AIPVKRGATGETKVETGNSNSPKSPTKGGC

SEQ ID NO: 238 - protein, 30 aa, Synthetic construct
AIPVKRGATGETKVFEGNSNSPKSPTKGGC

SEQ ID NO: 239 - protein, 30 aa, Synthetic construct
AIPVKRGATGETKVFTENSNSPKSPTKGGC

SEQ ID NO: 240 - protein, 30 aa, Synthetic construct
AIPVKRGATGETKVFTGESNSPKSPTKGGC

SEQ ID NO: 241 - protein, 30 aa, Synthetic construct
AIPVKRGATGETKVFTGNENSPKSPTKGGC

SEQ ID NO: 242 - protein, 30 aa, Synthetic construct
AIPVKRGATGETKVFTGNSESPKSPTKGGC

SEQ ID NO: 243 - protein, 30 aa, Synthetic construct
AIPVKRGATGETKVFTGNSNEPKSPTKGGC

SEQ ID NO: 244 - protein, 30 aa, Synthetic construct
AIPVKRGATGETKVFTGNSNSEKSPTKGGC

SEQ ID NO: 245 - protein, 30 aa, Synthetic construct
AIPVKRGATGETKVFTGNSNSPESPTKGGC

SEQ ID NO: 246 - protein, 30 aa, Synthetic construct
AIPVKRGATGETKVFTGNSNSPKEPTKGGC

SEQ ID NO: 247 - protein, 30 aa, Synthetic construct
AIPVKRGATGETKVFTGNSNSPKSETKGGC

SEQ ID NO: 248 - protein, 30 aa, Synthetic construct
AIPVKRGATGETKVFTGNSNSPKSPEKGGC

SEQ ID NO: 249 - protein, 30 aa, Synthetic construct
AIPVKRGATGETKVFTGNSNSPKSPTEGGC

SEQ ID NO: 250 - protein, 30 aa, Synthetic construct
AIPVKRGATGETKVFTGNSNSPKSPTKEGC

SEQ ID NO: 251 - protein, 30 aa, Synthetic construct
AIPVKRGATGETKVFTGNSNSPKSPTKGEC

SEQ ID NO: 252 - protein, 30 aa, Synthetic construct
AIPVKRGATGETKVFTGNSNSPKSPTKGGE

SEQ ID NO: 253 - protein, 29 aa, Synthetic construct
IPVKRGATGETKVFTGNSNSPKSPTKGGC

SEQ ID NO: 254 - protein, 28 aa, Synthetic construct
PVKRGATGETKVFTGNSNSPKSPTKGGC

SEQ ID NO: 255 - protein, 27 aa, Synthetic construct
VKRGATGETKVFTGNSNSPKSPTKGGC

SEQ ID NO: 256 - protein, 26 aa, Synthetic construct
KRGATGETKVFTGNSNSPKSPTKGGC

SEQ ID NO: 257 - protein, 25 aa, Synthetic construct
RGATGETKVFTGNSNSPKSPTKGGC

SEQ ID NO: 258 - protein, 24 aa, Synthetic construct
GATGETKVFTGNSNSPKSPTKGGC

SEQ ID NO: 259 - protein, 23 aa, Synthetic construct
ATGETKVFTGNSNSPKSPTKGGC

SEQ ID NO: 260 - protein, 22 aa, Synthetic construct
TGETKVFTGNSNSPKSPTKGGC

SEQ ID NO: 261 - protein, 21 aa, Synthetic construct
GETKVFTGNSNSPKSPTKGGC

SEQ ID NO: 262 - protein, 20 aa, Synthetic construct
ETKVFTGNSNSPKSPTKGGC

SEQ ID NO: 263 - protein, 19 aa, Synthetic construct
TKVFTGNSNSPKSPTKGGC

SEQ ID NO: 264 - protein, 18 aa, Synthetic construct
KVFTGNSNSPKSPTKGGC

SEQ ID NO: 265 - protein, 17 aa, Synthetic construct
VFTGNSNSPKSPTKGGC

SEQ ID NO: 266 - protein, 16 aa, Synthetic construct
FTGNSNSPKSPTKGGC

SEQ ID NO: 267 - protein, 15 aa, Synthetic construct
TGNSNSPKSPTKGGC

SEQ ID NO: 268 - protein, 29 aa, Synthetic construct
AIPVKRGATGETKVFTGNSNSPKSPTKGG

SEQ ID NO: 269 - protein, 28 aa, Synthetic construct
AIPVKRGATGETKVFTGNSNSPKSPTKG

SEQ ID NO: 270 - protein, 27 aa, Synthetic construct
AIPVKRGATGETKVFTGNSNSPKSPTK

SEQ ID NO: 271 - protein, 26 aa, Synthetic construct
AIPVKRGATGETKVFTGNSNSPKSPT

SEQ ID NO: 272 - protein, 25 aa, Synthetic construct
AIPVKRGATGETKVFTGNSNSPKSP

SEQ ID NO: 273 - protein, 24 aa, Synthetic construct
AIPVKRGATGETKVFTGNSNSPKS

SEQ ID NO: 274 - protein, 23 aa, Synthetic construct
AIPVKRGATGETKVFTGNSNSPK

SEQ ID NO: 275 - protein, 22 aa, Synthetic construct
AIPVKRGATGETKVFTGNSNSP

SEQ ID NO: 276 - protein, 21 aa, Synthetic construct
AIPVKRGATGETKVFTGNSNS

SEQ ID NO: 277 - protein, 20 aa, Synthetic construct
AIPVKRGATGETKVFTGNSN

SEQ ID NO: 278 - protein, 19 aa, Synthetic construct
AIPVKRGATGETKVFTGNS

SEQ ID NO: 279 - protein, 18 aa, Synthetic construct
AIPVKRGATGETKVFTGN

SEQ ID NO: 280 - protein, 17 aa, Synthetic construct
AIPVKRGATGETKVFTG

SEQ ID NO: 281 - protein, 16 aa, Synthetic construct
AIPVKRGATGETKVFT

SEQ ID NO: 282 - protein, 15 aa, Synthetic construct
AIPVKRGATGETKVF

SEQ ID NO: 283 - protein, 108 aa, Synthetic construct
GSYALGPYQISAPQLPAYNGQTVGTFYYVNDAGGLESKVFSSGGPTPYPNYANAGHVEGQSALFMRDNGISEGLVFHNNPEGTCGFCVNMTETLLPENAKMTVVPPEG

SEQ ID NO: 284 - protein, 107 aa, Synthetic construct
GSYALGPYQISAPQLPAYNGQTVGTFYYVNDAGGLESKVFSSGGPTPYPNYANAGHVEGQSALFMRDNGISEGLVFHNNPEGTCGFCVNMTETLLPENAKMTVVPPE

SEQ ID NO: 285 - protein, 106 aa, Synthetic construct
GSYALGPYQISAPQLPAYNGQTVGTFYYVNDAGGLESKVFSSGGPTPYPNYANAGHVEGQSALFMRDNGISEGLVFHNNPEGTCGFCVNMTETLLPENAKMTVVPP

SEQ ID NO: 286 - protein, 105 aa, Synthetic construct
GSYALGPYQISAPQLPAYNGQTVGTFYYVNDAGGLESKVFSSGGPTPYPNYANAGHVEGQSALFMRDNGISEGLVFHNNPEGTCGFCVNMTETLLPENAKMTVVP

SEQ ID NO: 287 - protein, 104 aa, Synthetic construct
GSYALGPYQISAPQLPAYNGQTVGTFYYVNDAGGLESKVFSSGGPTPYPNYANAGHVEGQSALFMRDNGISEGLVFHNNPEGTCGFCVNMTETLLPENAKMTVV

SEQ ID NO: 288 - protein, 103 aa, Synthetic construct
GSYALGPYQISAPQLPAYNGQTVGTFYYVNDAGGLESKVFSSGGPTPYPNYANAGHVEGQSALFMRDNGISEGLVFHNNPEGTCGFCVNMTETLLPENAKMTV

SEQ ID NO: 289 - protein, 102 aa, Synthetic construct
GSYALGPYQISAPQLPAYNGQTVGTFYYVNDAGGLESKVFSSGGPTPYPNYANAGHVEGQSALFMRDNGISEGLVFHNNPEGTCGFCVNMTETLLPENAKMT

SEQ ID NO: 290 - protein, 101 aa, Synthetic construct
GSYALGPYQISAPQLPAYNGQTVGTFYYVNDAGGLESKVFSSGGPTPYPNYANAGHVEGQSALFMRDNGISEGLVFHNNPEGTCGFCVNMTETLLPENAKM

SEQ ID NO: 291 - protein, 100 aa, Synthetic construct
GSYALGPYQISAPQLPAYNGQTVGTFYYVNDAGGLESKVFSSGGPTPYPNYANAGHVEGQSALFMRDNGISEGLVFHNNPEGTCGFCVNMTETLLPENAK

SEQ ID NO: 292 - protein, 99 aa, Synthetic construct
GSYALGPYQISAPQLPAYNGQTVGTFYYVNDAGGLESKVFSSGGPTPYPNYANAGHVEGQSALFMRDNGISEGLVFHNNPEGTCGFCVNMTETLLPENA

SEQ ID NO: 293 - protein, 98 aa, Synthetic construct
GSYALGPYQISAPQLPAYNGQTVGTFYYVNDAGGLESKVFSSGGPTPYPNYANAGHVEGQSALFMRDNGISEGLVFHNNPEGTCGFCVNMTETLLPEN

SEQ ID NO: 294 - protein, 109 aa, Synthetic construct
GSYALGPYQISAPQLPAYNGQTVGTFYYVNDAGGLESKVFSSGGPTPYPNYANAGHVEGQSALFMRDNGISEGLVFHNNPEGTCGFCVNMTETLLPENAKMTVVPPEGA

SEQ ID NO: 295 - protein, 110 aa, Synthetic construct
GSYALGPYQISAPQLPAYNGQTVGTFYYVNDAGGLESKVFSSGGPTPYPNYANAGHVEGQSALFMRDNGISEGLVFHNNPEGTCGFCVNMTETLLPENAKMTVVPPEGAI

SEQ ID NO: 296 - protein, 111 aa, Synthetic construct
GSYALGPYQISAPQLPAYNGQTVGTFYYVNDAGGLESKVFSSGGPTPYPNYANAGHVEGQSALFMRDNGISEGLVFHNNPEGTCGFCVNMTETLLPENAKMTVVPPEGAIP

SEQ ID NO: 297 - protein, 112 aa, Synthetic construct
GSYALGPYQISAPQLPAYNGQTVGTFYYVNDAGGLESKVFSSGGPTPYPNYANAGHVEGQSALFMRDNGISEGLVFHNNPEGTCGFCVNMTETLLPENAKMTVVPPEGAIPV

SEQ ID NO: 298 - protein, 113 aa, Synthetic construct
GSYALGPYQISAPQLPAYNGQTVGTFYYVNDAGGLESKVFSSGGPTPYPNYANAGHVEGQSALFMRDNGISEGLVFHNNPEGTCGFCVNMTETLLPENAKMTVVPPEGAIPVK

SEQ ID NO: 299 - protein, 114 aa, Synthetic construct
GSYALGPYQISAPQLPAYNGQTVGTFYYVNDAGGLESKVFSSGGPTPYPNYANAGHVEGQSALFMRDNGISEGLVFHNNPEGTCGFCVNMTETLLPENAKMTVVPPEGAIPVKR

SEQ ID NO: 300 - protein, 115 aa, Synthetic construct
GSYALGPYQISAPQLPAYNGQTVGTFYYVNDAGGLESKVFSSGGPTPYPNYANAGHVEGQSALFMRDNGISEGLVFHNNPEGTCGFCVNMTETLLPENAKMTVVPPEGAIPVKRG

SEQ ID NO: 301 - protein, 116 aa, Synthetic construct
GSYALGPYQISAPQLPAYNGQTVGTFYYVNDAGGLESKVFSSGGPTPYPNYANAGHVEGQSALFMRDNGISEGLVFHNNPEGTCGFCVNMTETLLPENAKMTVVPPEGAIPVKRGA

SEQ ID NO: 302 - protein, 117 aa, Synthetic construct
GSYALGPYQISAPQLPAYNGQTVGTFYYVNDAGGLESKVFSSGGPTPYPNYANAGHVEGQSALFMRDNGISEGLVFHNNPEGTCGFCVNMTETLLPENAKMTVVPPEGAIPVKRGAT

SEQ ID NO: 303 - protein, 118 aa, Synthetic construct
GSYALGPYQISAPQLPAYNGQTVGTFYYVNDAGGLESKVFSSGGPTPYPNYANAGHVEGQSALFMRDNGISEGLVFHNNPEGTCGFCVNMTETLLPENAKMTVVPPEGAIPVKRGATG

SEQ ID NO: 304 - protein, 119 aa, Synthetic construct
GSYALGPYQISAPQLPAYNGQTVGTFYYVNDAGGLESKVFSSGGPTPYPNYANAGHVEGQSALFMRDNGISEGLVFHNNPEGTCGFCVNMTETLLPENAKMTVVPPEGAIPVKRGATGE

SEQ ID NO: 305 - protein, 120 aa, Synthetic construct
GSYALGPYQISAPQLPAYNGQTVGTFYYVNDAGGLESKVFSSGGPTPYPNYANAGHVEGQSALFMRDNGISEGLVFHNNPEGTCGFCVNMTETLLPENAKMTVVPPEGAIPVKRGATGET

SEQ ID NO: 306 - protein, 121 aa, Synthetic construct
GSYALGPYQISAPQLPAYNGQTVGTFYYVNDAGGLESKVFSSGGPTPYPNYANAGHVEGQSALFMRDNGISEGLVFHNNPEGTCGFCVNMTETLLPENAKMTVVPPEGAIPVKRGATGETK

SEQ ID NO: 307 - protein, 122 aa, Synthetic construct
GSYALGPYQISAPQLPAYNGQTVGTFYYVNDAGGLESKVFSSGGPTPYPNYANAGHVEGQSALFMRDNGISEGLVFHNNPEGTCGFCVNMTETLLPENAKMTVVPPEGAIPVKRGATGETKV

SEQ ID NO: 308 - protein, 123 aa, Synthetic construct
GSYALGPYQISAPQLPAYNGQTVGTFYYVNDAGGLESKVFSSGGPTPYPNYANAGHVEGQSALFMRDNGISEGLVFHNNPEGTCGFCVNMTETLLPENAKMTVVPPEGAIPVKRGATGETKVF

SEQ ID NO: 309 - protein, 13 aa, Synthetic construct
GSGGGGSGGSGGS

SEQ ID NO: 310 - protein, 13 aa, Synthetic construct
GSGGGGSGGSEEE

SEQ ID NO: 311 - protein, 13 aa, Synthetic construct
GSGGGGSEEEEEE

SEQ ID NO: 312 - protein, 13 aa, Synthetic construct
GSGGEEEEEEEEE

SEQ ID NO: 313 - protein, 13 aa, Synthetic construct
GSGGGGSGEEEGS

SEQ ID NO: 314 - protein, 13 aa, Synthetic construct
GSGGGEEEEEEGS

SEQ ID NO: 315 - protein, 13 aa, Synthetic construct
GSEEEEEEEEEGS

SEQ ID NO: 316 - protein, 13 aa, Synthetic construct
GSGGGGSGGSDDD

SEQ ID NO: 317 - protein, 13 aa, Synthetic construct
GSGGGGSDDDDDD

SEQ ID NO: 318 - protein, 13 aa, Synthetic construct
GSGGDDDDDDDDD

SEQ ID NO: 319 - protein, 13 aa, Synthetic construct
GSGGGGSGDDDGS

SEQ ID NO: 320 - protein, 13 aa, Synthetic construct
GSGGGDDDDDDGS

SEQ ID NO: 321 - protein, 13 aa, Synthetic construct
GSDDDDDDDDDGS

SEQ ID NO: 322 - protein, 4 aa, Synthetic construct
SGGS

SEQ ID NO: 323 - protein, 5 aa, Synthetic construct
DDDGS

SEQ ID NO: 324 - protein, 8 aa, Synthetic construct
DDDDDDGS

SEQ ID NO: 325 - protein, 11 aa, Synthetic construct
DDDDDDDDDGS

SEQ ID NO: 326 - protein, 7 aa, Synthetic construct
SGDDDGS

SEQ ID NO: 327 - protein, 10 aa, Synthetic construct
SGDDDDDDGS

SEQ ID NO: 328 - protein, 13 aa, Synthetic construct
SGDDDDDDDDDGS

SEQ ID NO: 329 - protein, 5 aa, Synthetic construct
EEEGS

SEQ ID NO: 330 - protein, 8 aa, Synthetic construct
EEEEEEGS

SEQ ID NO: 331 - protein, 11 aa, Synthetic construct
EEEEEEEEEGS

SEQ ID NO: 332 - protein, 7 aa, Synthetic construct
SGEEEGS

SEQ ID NO: 333 - protein, 10 aa, Synthetic construct
SGEEEEEEGS

SEQ ID NO: 334 - protein, 13 aa, Synthetic construct
SGEEEEEEEEEGS

SEQ ID NO: 335 - protein, 108 aa, Synthetic construct
GSYALGPYQISAPQLPAYNGQTVGTFYYVNDAGGLESKVFSSGGPTPYPNYANAGHVAGQSALFMRDNGISEGLVFHNNPEGTCGFCVNMTETLLPENAKMTVVPPEG

SEQ ID NO: 336 - protein, 11 aa, Synthetic construct
YKCPECGKSFS

SEQ ID NO: 337 - protein, 11 aa, Synthetic construct
YACPECGKSFS

SEQ ID NO: 338 - protein, 11 aa, Synthetic construct
YACPECGRSFS

SEQ ID NO: 339 - protein, 11 aa, Synthetic construct
YACPECDRSFS

SEQ ID NO: 340 - protein, 11 aa, Synthetic construct
YACPECDRSFS

SEQ ID NO: 341 - protein, 11 aa, Synthetic construct
YACPECDRRFS

SEQ ID NO: 342 - protein, 13 aa, Synthetic construct
YACPVESCDRRFS

SEQ ID NO: 343 - protein, 13 aa, Synthetic construct
YACPVESCDRSFS

SEQ ID NO: 344 - protein, 13 aa, Synthetic construct
YACPVESCGKSFS

SEQ ID NO: 345 - protein, 11 aa, Synthetic construct
FACDICGRKFA

SEQ ID NO: 346 - protein, 5 aa, Synthetic construct
HIRTH

SEQ ID NO: 347 - protein, 354 aa, Synthetic construct
MLGFVGRVAAAPASGALRRLTPSASLPPAQLLLRAAPTAVHPVRDYAAQTSVDEMTKKFGTLTIHDTEKAAMAERPFQCRICMRNFSTSGSLSRHIRTHTGEKPFACDICGRKFAQSGSLTRHTKIHTGQKPFQCRICMRNFSRSDALSQHTKIHLRGSGSYALGPYQISAPQLPAYNGQTVGTFYYVNDAGGLESKVFSSGGPTPYPNYANAGHVEGQSALFMRDNGISEGLVFHNNPEGTCGFCVNMTETLLPENAKMTVVPPEGSGGSTNLSDIIEKETGKQLVIQESILMLPEEVEEVIGNKPESDILVHTAYDESTDENVMLLTSDAPEYKPWALVIQDSNGENKIKML

SEQ ID NO: 348 - protein, 365 aa, Synthetic construct
MLGFVGRVAAAPASGALRRLTPSASLPPAQLLLRAAPTAVHPVRDYAAQTSVDEMTKKFGTLTIHDTEKAAMAERPFQCRICMRNFSTSGSLSRHIRTHTGEKPFACDICGRKFAQSGSLTRHTKIHTGQKPFQCRICMRNFSRSDALSQHTKIHLRGSGGGGSGGSGGSGSYALGPYQISAPQLPAYNGQTVGTFYYVNDAGGLESKVFSSGGPTPYPNYANAGHVEGQSALFMRDNGISEGLVFHNNPEGTCGFCVNMTETLLPENAKMTVVPPEGSGGSTNLSDIIEKETGKQLVIQESILMLPEEVEEVIGNKPESDILVHTAYDESTDENVMLLTSDAPEYKPWALVIQDSNGENKIKML

SEQ ID NO: 349 - protein, 371 aa, Synthetic construct
MLGFVGRVAAAPASGALRRLTPSASLPPAQLLLRAAPTAVHPVRDYAAQDYKDDDDKVDEMTKKFGTLTIHDTEKAAMAERPFQCRICMRNFSTSGSLSRHIRTHTGEKPFACDICGRKFAQSGSLTRHTKIHTGQKPFQCRICMRNFSRSDALSQHTKIHLRGSGGGGSGGSGGSGSYALGPYQISAPQLPAYNGQTVGTFYYVNDAGGLESKVFSSGGPTPYPNYANAGHVEGQSALFMRDNGISEGLVFHNNPEGTCGFCVNMTETLLPENAKMTVVPPEGSGGSTNLSDIIEKETGKQLVIQESILMLPEEVEEVIGNKPESDILVHTAYDESTDENVMLLTSDAPEYKPWALVIQDSNGENKIKML

SEQ ID NO: 350 - protein, 124 aa, Synthetic construct
DGNEHWEVHPGLSLSDFEVVYGNNPHQIVKLRLDKEVGGSGGSMVQNDFIDSYTLCWLLRDDSGGGGSMVQNDFIDSYTLCWLLRDDDGNEHWEVHPGLSLSDFEVVYGNNPHQIVKLRLDKEV

SEQ ID NO: 351 - protein, 84 aa, Synthetic construct
MTNLSDIIEKETGKQLVIQESILMLPEEVEEVIGNKPESDILVHTAYDESTDENVMLLTSDAPEYKPWALVIQDSNGENKIKML

SEQ ID NO: 352 - protein, 112 aa, Synthetic construct
MTLELQLKHYITNLFNLPKDEKWHCESIEEIADDILPDQYVRLGALSNKILQTYTYYSDTLHESNIYPFILYYQKQLIAIGYIDENHDMDFLYLHNTIMPLLDQRYLLTGGQ

SEQ ID NO: 353 - protein, 130 aa, Synthetic construct
MNKNFDEVKADLRTVTGKKIEFKERLKNILRVQMNQLGFEDSYMIQVQVSSDQEEWVECHENMSLSDFEVMYGNISGEIKRMTVVKYEEANIEKLVELKFEYEYAKAHQEYIRAYTKLMSNTLYGRKPSL

SEQ ID NO: 354 - protein, 124 aa, Synthetic construct
MNEEKMHYRDAIKEVELTMMSLDSHFRTHKEFTDSYLLVLILEDVVGETRVEVSEGLTFDEASYIIGGTSDNILNMHMINYCEKNREEIYKWLKVSRVNTFKSNYAKMLLNTAYGKDLLKGVVK

SEQ ID NO: 355 - protein, 91 aa, Synthetic construct
MNNHFMSIGRNCSKCNNVRLNEDFSKSEEICNECFDKEERFVDSYTLIYITEDETGKRFEAILENQTIEETEIIYGNIIDKIIVWNVILTM

SEQ ID NO: 356 - protein, 1427 aa, Synthetic construct
MYEAARVTDPIDHTSALAGFLVGAVLGIALIAAVAFATFTCGFGVALLAGMMAGIGAQALLSIGESIGKMFSSQSGNIITGSPDVYVNSLSAAYATLSGVACSKHNPIPLVAQGSTNIFINGRPAARKDDKITCGATIGDGSHDTFFHGGTQTYLPVDDEVPPWLRTATDWAFTLAGLVGGLGGLLKASGGLSRAVLPCAAKFIGGYVLGEAFGRYVAGPAINKAIGGLFGNPIDVTTGRKILLAESETDYVIPSPLPVAIKRFYSSGIDYAGTLGRGWVLPWEIRLHARDGRLWYTDAQGRESGFPMLRAGQAAFSEADQRYLTRTPDGRYILHDLGERYYDFGQYDPESGRIAWVRRVEDQAGQWYQFERDSRGRVTEILTCGGLRAVLDYETVFGRLGTVTLVHEDERRLAVTYGYDENGQLASVTDANGAVVRQFAYTNGLMTSHMNALGFTSSYVWSKIEGEPRVVETHTSEGENWTFEYDVAGRQTRVRHADGRTAHWRFDAQSQIVEYTDLDGAFYRIKYDAVGMPVMLMLPGDRTVMFEYDDAGRIIAETDPLGRTTRTRYDGNSLRPVEVVGPDGGAWRVEYDQQGRVVSNQDSLGRENRYEYPKALTALPSAHIDALGGRKTLEWNSLGKLVGYTDCSGKTTRTSFDAFGRICSRENALGQRITYDVRPTGEPRRVTYPDGSSETFEYDAAGTLVRYIGLGGRVQELLRNARGQLIEAVDPAGRRVQYRYDVEGRLRELQQDHARYTFTYSAGGRLLTETRPDGILRRFEYGEAGELLGLDIVGAPDPHATGNRSVRTIRFERDRMGVLKVQRTPTEVTRYQHDKGDRLVKVERVPTPSGIALGIVPDAVEFEYDKGGRLVAEHGSNGSVIYTLDELDNVVSLGLPHDQTLQMLRYGSGHVHQIRFGDQVVADFERDDLHREVSRTQGRLTQRSGYDPLGRKVWQSAGIDPEMLGRGSGQLWRNYGYDAAGDLIETSDSLRGSTRFSYDPAGRLISRANPLDRKFEEFAWDAAGNLLDDAQRKSRGYVEGNRLLMWQDLRFEYDPFGNLATKRRGANQTQRFTYDGQDRLITVHTQDVRGVVETRFAYDPLGRRIAKTDTAFDLRGMKLRAETKRFVWEGLRLVQEVRETGVSSYVYSPDAPYSPVARADTVMAEALAATVIDSAKRAARIFHFHTDPVGAPQEVTDEAGEVAWAGQYAAWGKVEATNRGVTAARTDQPLRFAGQYADDSTGLHYNTFRFYDPDVGRFINQDPIGLNGGANVYHYAPNPVGWVDPWGLAGSYALGPYQISAPQLPAYNGQTVGTFYYVNDAGGLESKVFSSGGPTPYPNYANAGHVEGQSALFMRDNGISEGLVFHNNPEGTCGFCVNMTETLLPENAKMTVVPPEGAIPVKRGATGETKVFTGNSNSPKSPTKGGC

SEQ ID NO: 357 - protein, 29 aa, Synthetic construct
MSVLTPLLLRGLTGSARRLPVPRAKIHSL

SEQ ID NO: 358 - protein, 83 aa, Synthetic construct
TNLSDIIEKETGKQLVIQESILMLPEEVEEVIGNKPESDILVHTAYDESTDENVMLLTSDAPEYKPWALVIQDSNGENKIKML

SEQ ID NO: 359 - protein, 384 aa, Synthetic construct
MLGFVGRVAAAPASGALRRLTPSASLPPAQLLLRAAPTAVHPVRDYAAQDYKDDDDKVDEMTKKFGTLTIHDTEKGSLQKKLEELELDAAMAERPFQCRICMRNFSTSGSLSRHIRTHTGEKPFACDICGRKFAQSGSLTRHTKIHTGQKPFQCRICMRNFSRSDALSQHTKIHLRGSGGGGSGGSGGSGSYALGPYQISAPQLPAYNGQTVGTFYYVNDAGGLESKVFSSGGPTPYPNYANAGHVEGQSALFMRDNGISEGLVFHNNPEGTCGFCVNMTETLLPENAKMTVVPPEGSGGSTNLSDIIEKETGKQLVIQESILMLPEEVEEVIGNKPESDILVHTAYDESTDENVMLLTSDAPEYKPWALVIQDSNGENKIKML

SEQ ID NO: 360 - protein, 16 aa, Synthetic construct
SGSETPGTSESATPES

SEQ ID NO: 361 - protein, 1425 aa, Burkholderia gladioli
MYEAARVTDPIEHTSALAGFLVGAVLGIALIAAVAFATFTCGFGVALLAGMAAGIGAQVLLSLGESIGKMFSSQSGAITLGSPNVYVNGKQAAYATLSSVTCSKHNPTPLVAQGSTNIFINGKPAARKDDKITCGAAISDGSHDTYFHGGIQTCLPIDDEVPPWLRTATDWAFALAGLVGGLGGLLKEAGGLSHAVMPCAAKFIGGYVLGEAASRYVIGPAINSAIGGMFGNPVDVTTGRKILPAESETDYVVPSPMPVAIRRFYSSDLDYVGTLGRGWVLPWELRLHARDGRLWYTDAQGRESGFPILKPGQAAFSEADQRYLTCTPDGRYILHDVGETYYDFGRYEPGSGRIGWVRRIEDQAGQWCQFERDSRGRVREIQTCGGLLAVLDYEPEHERLAEVSLVSGDQRRLVVAYGYDENGQMASVTDANGAVVRRFTYADGRMTSHSNALGFTSGYTWKVIDGTPRVVATHTSEGEAWAFEYDIEGRRTHVRHADGRHAQWRYDAQFQIVEYLDFDGRRYGLKYNAAGMPVMLTLPGERTVMFEYDDAGRIVAETDPLGRTTKTRYDGNSMRPVEIILPDGSAWHAEYDRQGRLLVTRDPLDRENRYEYPEALSALPVAHVDALGGRKTFEWNRLGELVAYTDCSGKTTRNFFDAFGLPLARENALGHRVSFDLRPTGETRRVTYPDGSSESYEYDAAGLMIRHIGLGGRMQTLQRNARGQLVEAVDPAGRRTRYHYDAEGRLRELQQAHARYAFAYSAGGRLVSETRPDGVLRRFEYGEAGDLAALEIVGTADDCAPNDRPVRAIRFERDRMGNLCVQHTPTEVTRYERDAGGRLLEVASVPTAAGLALGIAPDTLTFEYDKAGRLSAEHGANGSVQYTLDALDNVLKLALPHEQTLQMLRYGSGHVHQIRHGDQVVSDFERDDLHRELTRTQGPLTERTAYDLLGRKIWQSAGFQPDALARGQGQLWRNYGYDAAGELVESHDSLRGSTQFSYDPAGYLTQRVNTADRQLESFAWDAAGNLLDDAQRSSRGYVEGNRLRMWQNLRFDYDAFGNLATKLRGANQRQQFTYDGQDRLVAVRTQGARGVVETRFAYDPLGRRIAKTDRTLDVRGVTLREETKRFVWEGLRLAQEVRDTGVSSYVYSPDAPYMPAARVDAVKAEALANAAIDKARQATRIYHFHTDVSGAPQEATNEAGDIVWAGQYSAWGKVAPNQHAPARIDQPLRYAGQYADDSTELHYNTFRFYDPDVGRFINQDPIGLMGGLNLYQYAPNSIAWTDWWGLAGSYTLGSYQISAPQLPAYNGQTVGTFYYVNDAGGLESRTFSSGGPTPYPNYANAGHVEGQSALFMRDNGISDGLVFHNNPEGTCGFCVNMTETLLPENSKLTVVPPEGSIPVKRGATGETRTFTGNSKSPKSPVKGGC

SEQ ID NO: 362 - DNA, 4278 bp, Burkholderia gladioli
  source 1..4278: mol_type = genomic DNA
gtgtacgaagcggcccgcgtcacggatccgatcgagcacaccagcgcgctggccggcttcctggtgggcgccgtgctcggtatcgccctgattgctgccgtggcgttcgccacgttcacctgcggcttcggcgtggcactgctggccggcatggcggccggcatcggcgcgcaggtgctgttgtcgttaggggaatcgatcgggaagatgttcagttcgcaatccggcgcgatcacgctcggctcgccgaacgtctacgtgaacggcaagcaggccgcctacgccacgctcagcagcgtgacgtgcagcaagcacaacccgacgccgctcgtcgcgcagggctccaccaacatcttcatcaacggcaagccggccgcgcgcaaggacgacaagatcacctgcggcgcggccatctcggacggctcgcacgacacctacttccacggaggcatccagacctgcctgccgatcgacgacgaagtgccgccgtggctgcgcaccgccaccgactgggcgttcgcgctggccgggctggtgggcgggctcggcggcctactcaaggaagcgggcgggctgtcgcacgcggtgatgccgtgcgcggcgaagttcatcggcggctacgtgctcggcgaggcggcgagccgctacgtgatcggcccggccatcaacagcgcgatcggcgggatgttcggcaacccggtagacgtcaccactgggcgcaagatcctccctgccgaatcggaaaccgattacgtcgtgcccagcccgatgccggtggcgatccggcgcttctattcgagcgacctcgattacgtcggcacgcttgggcgcggctgggtgctgccgtgggagctgcgcctgcacgcgcgtgacggtcggctctggtacaccgacgcgcaggggcgcgagagcggcttcccgatcctgaaaccgggccaggccgcgttcagcgaggccgatcagcgctatctgacctgcacgccggatggccgctacatcctccacgacgtcggcgaaacctattacgacttcggccgctacgagccgggctcgggccgcatcggctgggtgcgccggatcgaggatcaggccggccagtggtgccagttcgagcgcgacagccgtggccgcgtgcgtgaaatccagacctgcggcggcttgctggccgtgctcgattacgagccggagcacgagcggctcgccgaggtgtcgctcgtcagcggcgatcagcgccgcctcgtcgtggcctacggctacgacgaaaacggccagatggcctccgtgaccgacgcgaacggcgcggtggtgcgccgcttcacctatgccgacgggcgcatgacgagccattcgaacgcgctcggtttcacgtcgggctatacgtggaaggtcatcgacggcacgccgcgagtggtcgccacccacaccagcgagggcgaggcctgggcgttcgagtacgacatcgaaggccgccgcacccatgtgcggcatgccgacggccgccacgcgcaatggcgctacgacgcgcaattccagatcgtcgagtacctcgatttcgacggccgtcgctacgggctcaagtacaacgctgccggcatgcccgtgatgctgacgctgcccggcgaacgaaccgtgatgttcgagtacgacgacgccggccgcatcgtcgccgaaaccgatcccctcggccgcaccacgaaaacgcgctacgacggcaacagcatgcggcccgtcgagatcatcttgcccgacggcagcgcctggcacgccgaatacgaccggcagggccggctgctcgtcacccgtgatccgctcgaccgggagaatcgctacgaatatccggaggcactgagcgcgctcccggtggcgcatgtcgatgcgctgggcgggcgcaagacgttcgagtggaaccggctcggcgagctggtggcctacaccgattgctcgggcaagaccacgcgcaattttttcgatgcattcggcctgccgctcgcgcgcgagaacgcgctcgggcaccgcgtgtcgttcgatctgcgcccgaccggcgagacgcgccgcgtcacctatcccgacggcagttccgaaagctacgaatacgacgccgccgggctgatgatccggcacatcgggctgggcggccggatgcagacgttgcagcgcaatgcgcgcgggcaactcgtcgaggcggtcgatccggccgggcggcgaacccgctaccactacgacgccgaagggcggctgcgcgagctgcaacaggcccacgcgcgctacgcattcgcgtacagcgcaggcgggcggcttgtcagcgaaacgcggcccgacggcgtgctgcgccgcttcgaatacggcgaggccggcgatctggcggcgctcgagatcgtcggaacggccgatgattgcgctccaaacgatcgcccggttcgcgcgatccgcttcgagcgcgaccggatgggtaacctgtgcgtgcagcacacgcctaccgaggtgacgcgctacgagcgcgacgccggcggccgcctgctcgaagtcgcgagcgtgccgaccgcggccggactggcgctcggcatcgcgcccgacacgctgaccttcgaatacgacaaggccgggcggctgagcgccgaacacggcgcgaacggcagcgtccagtacacgctcgacgcgctcgacaacgtgttgaagctcgccttgccgcacgaacagacgctgcagatgctgcgctacggctcggggcacgtgcaccagattcgccacggcgaccaggtcgtcagcgatttcgagcgcgacgacctgcatcgcgagttgacgcgcacgcagggccccctgaccgagcggaccgcctacgacctgctgggccgcaagatctggcaatcagccggcttccagcccgacgcgcttgcgcgtgggcagggccagctgtggcgcaactacggctacgacgccgccggggaactggtcgagagccacgacagcctgcgcggcagcacgcagttcagctacgatccggccggctatctgacgcagcgcgtgaacaccgccgaccggcagctcgaatcgttcgcctgggacgccgccggcaacctgctcgacgatgcgcaacgcagcagccgcggctatgtcgagggcaaccggctgcgcatgtggcagaacctgcgcttcgactacgacgcgttcggcaatctcgcgaccaagctgcgcggcgcgaatcagcgccagcagttcacgtacgatgggcaggatcggctcgtggccgtgcgcacgcagggcgcgcgcggcgtggtggagacgcgtttcgcctacgatccgctcgggcggcgcatcgccaagaccgataggacactcgacgtgcgcggcgtaacgctgcgcgaggaaacgaagcggttcgtatgggaagggctgcggctcgcgcaggaggtgcgcgacaccggcgtgagcagctacgtgtacagcccggatgcgccttacatgcccgcggcgcgggtcgatgcggtgaaagccgaagcgctcgcaaacgccgcgatcgacaaggccagacaggcgacgcggatctatcactttcataccgatgtgtcgggcgcaccgcaagaagcgacgaacgaggccggcgacattgtttgggccggccaatactcagcctggggcaaggtggcgccgaaccagcatgccccagcccggatcgatcagccgctccgctacgccggacaatatgccgatgacagtaccgagctgcactacaacacgtttcgtttctacgatccggatgtcggccggtttatcaatcaggatccaatcgggttgatgggggggctgaatctttaccaatatgcacccaactcaatcgcgtggaccgactggtgggggctggccggcagctatacgctcggttcctatcaaatttctgctcctcaacttcccgcctacaatgggcagactgttgggaccttctactatgtaaacgacgcgggcgggctcgaatcgaggacattctcttctggagggccgaccccttatccaaattatgccaatgccgggcacgtggaaggccagtccgcactgttcatgagggataacggaatttcagacggactggttttccacaacaaccctgagggtacttgcggattctgcgtcaatatgaccgaaacgcttttgcctgaaaattccaaacttaccgtcgttccgcccgagggctcgattccggtcaagcggggcgcgacgggcgaaacgagaacatttacagggaacagcaagtctccgaagtcccctgtcaaaggaggatgttga

SEQ ID NO: 363 - protein, 1425 aa, Burkholderia glumae
MYEAARVTDPIEHTSALTGFLVGAVLGIALIAAVAFATFTCGFGVALLAGMAAGIGAQVLLSLGESIGKMFSSQSGAITLGSPNVYVNGKPTAYAMLSSVTCSKHNPTPLVAQGSTNIFINGKPAARKDDKITCGATISDGSHDTYFHGGTQTCLPIDDEVPPWLRTATDWAFALAGLVGGLGGLLKEAGGLSRAVMPCAAKFIGGYVLGEAASRYVVGPAINSAIGGMFGNPVDVTTGRKILLAESETDYVVPSPMPVAIRRFYSSDLDYVGTLGRGWVLPWELRLHARDGRLWYTDAQGRESGFPMLQPGHAAFSEADQRYLTCTPDGRYILHDLGETYYDFGHYEPGSGRIGWVRRIEDQAGQWCQFERDSRGRVREIQTCGGLLAVLDYEPEHGRLAGVSLVSGDQRRLVVAYGYDEHGQMASVTDANGALVRRFTYADGRMTSHSNALGFTSGYTWQAVGGAPRVVATHTSEGEAWAFEYDIEGRRTHVRHADGRHAQWRYDAQFQIVEYLDFDGRRYGLKYNDAGMPVMLTLPGERTVTFEYDDAGRIVAETDPLGRTTKTRYDGNSRRPVEIIAPDGSAWHAEYDRQGRLLATRDPLDRENRYEYPKALSALPIAHVDALGGRKTFEWNRLGELVAYTDCSGKTTRNFYDAFGLPLARENALGHRVTFDLRPTGEARRVTYPDGSTESYEYDAAGLMIRHVGLGGRTQIALRNARGQIVEAVDPAGRRTCYRYDAEGRLRELQQGHARYAFTYSAGGRLTSETRPDGVRRRFEYGEAGDLAALDIVGAADDATANDRPVRTIRFERDRMGNLCAQHTPTEVTRYTRDTGGRLLEVACVPTAAGLALGIAPDTLTFEYDKAGRLSAEHGANGSVRYTLDALDNVMKLALPHEQTLQMLRYGSGHVHQIRCGDQVVSDFERDDLHRELTRTQGRLTERTAYDLLGRKIWQSAGFQPDALARGQGQVWRNYGYDAAGELAESHDSLRGSTQFSYDPAGYLTQRVNTADRQLESFAWDAAGNLLDDAQRRSRGYVEGNRLRMWQNLRFEYDPFGNLATKLRGANQRQQFTYDGQDRLVAVRTQDARGVVETRFAYDPLGRRIAKTDIVRDARGVALREETKRFVWEGLRLAQEVRDTGVSSYVYSPDAPYTPAARVDAVLAEAMAAAAIEQARQATRIYHFHTDVSGAPQEATNEAGDIVWAGQYSAWGKVAPNQHAPARIDQPLRYAGQYADDSTELHYNTFRFYDPDVGRFINQDPIGLMGGLNLYQYAPNSIAWTDWWGLAGSYTLGSYQISAPQLPAYNGQTVGTFYYVNGAGGLESRTFSSGGPTPYPNYANAGHVEGQSALFMRDNGISDGLVFHNNPEGTCGFCVNMTETLLPENSKLTVVPPEGAIPVKRGATGETRTFTGNSKSPKSPVKGEC

SEQ ID NO: 364 - protein, 1425 aa, Burkholderia glumae; identical to SEQ ID NO: 363

SEQ ID NO: 365 - DNA, 4278 bp, Burkholderia glumae
  source 1..4278: mol_type = genomic DNA
gtgtacgaagcggcccgcgtcaccgacccgatcgaacacaccagcgcgctgaccggctttctggtgggcgccgtgctcggcattgccctgatcgccgcggtggcgttcgccaccttcacctgcggcttcggcgtggcgctgctggccggcatggccgccggcatcggcgcgcaggtgctgttgtcgttaggagaatcgatcgggaagatgttcagttcgcaatccggcgcgatcacgctcggctcgccgaacgtctatgtgaacggcaagccgaccgcctacgccatgctcagcagcgtgacgtgcagcaagcacaacccgacgccgctcgtcgcgcaggggtccaccaacatcttcatcaacggcaagccggccgcccgcaaggacgacaagatcacctgcggcgcgaccatctccgacggctcgcacgacacctatttccacggcggcacccagacctgcctgccgatcgacgacgaagtgccgccgtggctgcgcaccgccaccgactgggcgttcgcgctggccgggctggtgggcgggctcggcggcctgctcaaggaagcgggcgggctgtcgcgcgcggtgatgccgtgcgcggcgaagttcatcggcggctacgtgctcggcgaggcggcgagccgctacgtggtcggcccggccatcaacagcgcgatcggcgggatgttcggcaacccggtggacgtcaccaccgggcgcaagatcctgctggcggaatcggaaaccgattacgtggtgcccagcccgatgccggtggcgatccggcgcttctattcgagcgacctcgactacgtcggcacgctcgggcgcggctgggtgctgccgtgggaactgcggctgcacgcgcgcgacgggcggctctggtacaccgacgcgcaggggcgcgagagcggcttcccgatgctccagccgggccatgccgcgttcagcgaggccgaccagcgctatctgacctgcaccccggatggccgctacatcctgcacgacctcggcgaaacctattacgacttcggccactacgagccgggctcgggccgcatcggctgggtgcgccgcatcgaggatcaggccggccagtggtgccagttcgagcgcgacagccgcggccgcgtgcgcgaaatccagacctgcggcggcttgctggccgtgctcgattacgagccggaacacgggcggctcgccggggtgtcgctcgtcagcggggatcagcgccgcctcgtggtggcttacggctatgacgagcacggccagatggcgtccgtgaccgatgcgaacggcgcgctggtgcgccgcttcacctatgccgacgggcgcatgacgagccattcgaacgcgctcggcttcacgtcgggctatacgtggcaagccgtcggcggcgcgccgcgggtggttgccacccacaccagcgagggcgaggcctgggccttcgagtacgacattgaaggacgccgcacccacgtgcgtcacgccgacggccgccacgcgcaatggcgctacgacgcgcaattccagatcgtcgagtacctcgatttcgacggccggcgctacgggctcaagtacaacgacgccggcatgcccgtgatgctgacgctgcccggcgaacggaccgtgacgttcgagtacgacgatgccggccgcatcgtcgccgaaaccgatccactcggccgcaccacgaaaacgcgctacgacggcaacagcaggcggcccgtcgagatcatcgcgcccgacggcagcgcctggcacgccgaatacgaccggcaaggccggctgctcgccacccgcgatccgctcgaccgggaaaaccgctacgaatacccgaaggcgctcagcgcgctgccgatcgcgcacgtcgatgcgctgggcgggcgcaagacgttcgagtggaaccggctcggcgagctggtggcctataccgattgctcgggcaagaccacacgcaatttttacgacgcattcggtctgccgctcgcgcgcgagaacgcgctcggccaccgcgtgacgttcgacctgcgcccgaccggcgaggcgcggcgcgtcacctatcccgacggcagtacagaaagctacgaatacgacgccgccgggctgatgatccggcacgtcgggctgggcggccggacgcagattgcgctgcgcaacgcgcgtgggcagatcgtggaggcggtcgatccggccggacggcgcacctgctaccgctacgacgccgaggggcggctgcgcgagctgcaacaggggcacgcgcgttacgcgttcacctacagcgcgggcgggcggctcaccagcgaaacccggcccgacggcgtgcggcgccgcttcgaatacggcgaggccggcgatctggcggcgctcgacatcgtcggcgcggccgacgacgccacggcgaacgatcgtccggttcgcaccatccgcttcgagcgcgaccgcatgggcaatctgtgcgcgcagcacacgcccaccgaggtgacgcgctacacgcgcgacaccggcggccgcctgctcgaagtcgcatgcgtgccgaccgcggccgggctggcgctcggcatcgcgcccgacacgctgaccttcgaatacgacaaggccgggcggctgagtgccgaacacggcgcgaacggcagcgtccgatacacgctcgacgcgctcgacaacgtgatgaagctcgccctgccgcacgagcagacgctgcagatgctgcgctacggctcggggcacgtgcatcagatccgctgcggcgaccaggtggtcagcgatttcgagcgcgacgacctgcatcgcgagctgacgcgcactcagggccgcctgaccgagcgtaccgcctacgacctgctgggccgcaagatctggcaatcggccggcttccagcccgacgcgcttgcgcgcgggcagggccaggtgtggcgcaactacggctacgacgccgccggcgaactggccgagagccacgatagcctgcgcggcagcacgcagttcagctacgatccggccggctatctgacgcagcgcgtcaataccgccgaccggcagctcgaatcgttcgcctgggatgccgccggcaacctgctcgacgatgcgcagcgccgcagccgcggttatgtcgagggcaaccggctgcgcatgtggcagaacctgcgcttcgaatacgacccgttcggcaatctcgcgaccaagctgcgcggcgcgaaccagcgccagcagttcacttacgacgggcaggatcggctcgtggcggtgcgcacgcaggacgcgcgcggcgtggtggagacgcgtttcgcctacgatccgctggggcggcgcatcgccaagacggatattgtgcgcgacgcgcgcggcgtagcgctgcgcgaggaaacgaagcggttcgtgtgggaggggctgcggctcgcgcaggaggtgcgcgacacgggcgtgagcagctacgtgtacagcccggacgcgccctatacgcccgcggcgcgcgtggatgccgtgctggccgaggccatggccgccgctgccatcgagcaggccagacaggcgacgcggatctatcactttcataccgatgtgtcgggcgcaccgcaagaagcgacgaacgaggctggcgacattgtttgggccggccaatactcagcctggggcaaggtggcgccgaaccagcatgcccccgcccggatcgatcagccgctccgctacgccggacaatatgccgacgacagtaccgagctgcactacaacacgtttcgtttctacgatccggacgtcggccggtttatcaatcaggatccaatcgggttgatgggggggctgaatctttaccaatatgcacccaactcgatcgcatggaccgactggtgggggctggccggcagctatacgctcggttcctatcaaatttctgcgcctcaacttccggcctacaatggacagactgttgggaccttctactacgtgaacggcgcgggcgggctcgaatcgaggacattctcttccggagggccgaccccttatccaaattatgccaatgccgggcacgtggagggccagtccgcgctgttcatgagggataacggaatttcagacggactggttttccacaacaaccctgagggcacttgcggattctgcgttaatatgaccgaaacgcttttgcctgaaaattccaaacttaccgtcgttccgcccgagggcgcgatcccggtcaagcggggcgcgacgggcgaaacgagaacatttacggggaacagcaagtctccgaagtcccctgtcaaaggagaatgttga

SEQ ID NO: 366 - DNA, 4278 bp, Burkholderia glumae; identical to SEQ ID NO: 365
  source 1..4278: mol_type = genomic DNA

SEQ ID NO: 367 - protein, 2358 aa, unidentified
  source 1..2358: note = Streptomyces rubrolavendulae
MSSSDAGRAFGVPENVLARFTRYPGGARRRAGRTARARRLGIVLSAVLSATLLPAEAWAIAPPAPRTGPTLDALQQEEEVDPDPAAMEELDDWDGGPVEPPADYTPTEVTPPTGGTAPVPLDSAGEELVPAGTLPVRIGQASPTEEDPAPPAPSGTWDVTVEPRATTEAAAVDGAIIKLTPPASGSTPVDVELDYGRFEDLFGTEWSSRLKLTQLPECFLTTPELEECGTPITIPTSNDPATGTVRATVDPADGQPQGLAAQSGGGPAVLAATDSASGAGGTYKATSLSATGSWTAGGSGGGFSWSYPLTIPDTPAGPAPKISLSYSSQSVDGRTSVANGQASWIGDGWDYHPGFVERRYRSCNDDRSGTPNNDNSADKEKSDLCWASDNVVMSLGGSTTELVRDDTTGTWVAQNDTGARIEYKDKDGGALAAQTAGYDGEHWVVTTRDGTRYWFGRNTLPGRGAPTNSALTVPVFGNHTGEPCHAATYAASSCTQAWRWNLDYVEDVHGNAMVVDWKKEQNRYAKNEKFKAAVSYDRDAYPTQILYGLRADDLAGPPAGKVVFHAAPRCLESAATCSEAKFESKNYADKQPWWDTPATLHCKAGDENCYVTSPTFWSRVRLSAIETQGQRTPGSTALSTVDRWTLHQSFPKQRTDTHPPLWLESITRVGFGRPDASGNQSSKALPAVTFLPNKVDMPNRVLKSTTDQTPDFDRLRVEVIRTETGGETHVTYSAPCPVGGTRPTPASNGTRCFPVHWSPDPAAFSDENLDKSGYEPPLEWFNKYVVTKVTEMDLVAEQPSVETVYTYEGDAAWAKNTDEYGKPALRTYDQWRGYASVVTRTGTTANTGAADATEQSQTRTRYFRGMSGDAGRAKVHVTLTDVTGTATTVEDLLPYQGMAAETLTYTKAGGDVAARELAFPYSRKTASRARPGLPALEAYRTGTTRTDSIQHISGDRTRAAQNHTTYDDAYGLPTQTYSLTLSPNDSGTLVAGDERCTVTTYVHNTAAHIIGLPDRVRATTGDCAAAPNATTGQIVSDSRTAYDALGAFGTAPVKGLPVQVDTISGGGTSWITSARTEYDALGRATKVTDAAGNSTTTTYSPATGPAFEVTVTNAAGHATTTTLDPGRGSALTVTDQNGRKTTSTYDELGRATGVWTPSRPVNQDASVRFVYQIEDSKVPAVHTRVLRDAGTYEESIELYDGFLRPRQTQREALGGGRIVTETLYNANGSAKEVRDGYLAEGEPARELFVPLSLDQVPSATRTAYDGLGRPVRTTTLHRGVPRHSATTAYGGDWELSRTGMSPDGTTPLSGSRAVKATTDALGRPARIQHFTTQNVSAESVDTTYTYDPRGPLAQVTDAQQNTWTYTYDARGRKTSSTDPDAGAAYFGYNALDQQVWSKDNQGRLQYTTYDVLGRQTELRDDSASGPLVAKWTFDTLPGAKGHPVASTRYNDGAAFTSEVTGYDTEYRPTGNKVTIPSTPMTTGLAGTYTYASTYTPTGKVQSVDLPATPGGLAAEKVITRYDGEDSPTTMSGLAWYTADTFLGPYGEVLRTASGEAPRRVWTTNVYDEDTRRLTRTTAHRETAPHPVSTTTYGYDTVGNITSIADQQPAGTEEQCFSYDPMGRLVHAWTDGNSAVCPRTSTAPGAGPARADVSAGVDGGGYWHSYAFDAIGNRTKLTVHDRTDAALDDTYTYTYGKTLPGNPQPVQPHTLTQVDAVLNEPGSRVEPRSTYAYDTSGNTTQRVIGGDTQTLAWDRRNKLTSVDTNNDGTPDVKYLYDASGNRLVEDDGTTRTLFLGEAEIVVNTAGQAVDARRYYSSPGAPTTIRTTGGKTTGHKLTVMLSDHHSTATTAVELTDTQPVTRRRFDPYGNPRGTEPTTWPDRRTYLGVGIDDPATGLTHIGAREYDASTGRFISVDPVMDLTDPLQMNGYTYANADPINNSDPTGLLLDARGGGTQKCVGTCVKDVTNRKGIPLPPGEEWKHEGEAQTDFNGDGFITVFPTVNVPAKWKKAKKYTEAFYKAVDTACFYGRESCADPEYPSRAHSINNWKGKACKAVGGKCPERLSWGEGPAFAGGFAIAAEEYAGRGGYRGGGARRGSPCKCFLAGTEVLMADGSTKSIEDIKLGDEVVATDPVTGEAGAHPVSALIATENDKRFNELVIITSEGVERLTATHEHPFWSPSEGEWLEAGELRTGMTLRSDSGETLVVAGNRAFTQRARTYNLTVADLHTYYVLAGQTPVLVHNANCGPHLKDLQKDYPRRTVGILDVGTDQLPMISGPGGQSGLLKNLPGRTKANGEHVETHAAAFLRMNPGVRKAVLYIDYPTGTCGTCRSTLPDMLPEGVQLWVISPRRTEKFTGLPD

SEQ ID NO: 368 - DNA, 7077 bp, unidentified
  source 1..7077: mol_type = genomic DNA; note = Streptomyces rubrolavendulae
atgtcctcgtccgatgcgggacgcgccttcggcgtgcccgaaaacgtcctggcgcgtttcacgcggtatcccggcggggcgcgacgccgtgccgggcgcacggcgcgcgcccggcgcctgggcatcgtgctgtccgccgtcctctcggcgaccctgctgcccgccgaggcatgggccatcgcgcccccggcgccgcgcaccggtccgaccctggacgccctccagcaggaggaggaggtcgatccggacccggccgccatggaagagctggacgactgggacggtgggccggtcgagcccccggccgactacacccccaccgaggtcacgcctcccaccggcggcaccgccccggtgccgctggacagcgcgggcgaggaactggtcccggccgggaccctgcccgtgcgcatcggccaggcgtcccccaccgaggaggacccggcacccccggcacccagcggcacgtgggacgtcaccgtggagccccgcgccaccaccgaggcggccgccgtggacggcgccatcatcaagctcaccccgcccgccagcggctccacaccggtcgacgtggaactcgactacggccggttcgaggacctgttcggcaccgagtggtcctcccggctcaagctgacgcagctcccggagtgcttcctcacgacgcccgagctggaggagtgcggcacccccatcaccatcccgacgagcaacgacccggccaccgggacggtccgggccaccgtcgacccggccgacgggcagccgcagggcctggccgcgcagtcgggcggcggtcccgccgtcctcgccgcgaccgactcggcgtccggcgccggcggcacgtacaaggcgacctccctctcggccaccggctcctggacggccggcggcagcggcggcggcttctcctggtcgtatccgctcaccatcccggacaccccggccggccccgcgccgaagatctccctgtcgtactcctcccagtccgtcgacggccgcacctccgtcgccaacggccaggcgtcgtggataggcgacggctgggactaccaccccggcttcgtcgagcgccgctaccgctcctgcaacgacgaccgctccggcaccccgaacaacgacaacagtgcggacaaggagaagtccgacctgtgctgggcgagcgacaacgtcgtgatgtcgctcggcggctccaccaccgaactcgtccgcgacgacacgaccggcacgtgggtcgcgcagaacgacaccggtgcccggatcgagtacaaggacaaggacggcggagccctggccgcccagaccgccggctacgacggcgagcactgggtcgtcaccacccgcgacggaacccgctactggttcggccgcaacaccctccccggccgcggcgcccccacgaactccgccctcaccgtccccgtcttcggcaaccacaccggcgagccctgccacgccgccacctacgccgcctcctcctgcacccaggcgtggcgctggaacctcgactacgtcgaggacgtccacggcaacgcgatggtcgtcgactggaagaaggagcagaaccggtacgcgaagaacgagaagttcaaggcggctgtctcctacgaccgcgacgcgtatccgacgcagatcctctacggcctgcgcgccgacgacctggcgggcccgcccgccggcaaggtcgtcttccacgccgccccgcgctgcctcgaaagcgcggccacctgctccgaagccaagttcgagtccaagaactacgcggacaagcagccctggtgggacacaccggccaccctgcactgcaaggccggtgacgagaactgctacgtcacctcgccgacgttctggagccgcgtccgcctgtcggcgatcgagacgcagggtcagcgcacgcccggctcgacggcgctgtccacggtcgaccgctggaccctgcaccagtcgttcccgaagcagcgcaccgacacccacccgccgctctggctggagtcgatcacccgcgtgggcttcggccggccggacgcctccggcaaccagtcgagcaaggccctcccggcggtgaccttcctgcccaacaaggtcgacatgccgaaccgcgtgctgaagagcacgacggaccagacgcccgatttcgaccgcctgcgcgtcgaggtcatccgcacggagaccggcggcgagacccatgtgacgtactccgccccctgccccgtcggcggcacccgccccaccccggcctccaacggcacccgctgcttcccggtccactggtcccccgacccggcggccttctccgacgagaacctggacaagagcggctacgagccgcccctcgagtggttcaacaagtacgtcgtcaccaaggtcaccgagatggacctcgtggcggagcagcccagcgtcgagaccgtctacacctacgagggcgacgccgcctgggcgaagaacaccgacgagtacggcaagcccgccctgcgcacctacgaccagtggcgcggctacgcgagcgtcgtcacccgcacgggcaccacggccaacaccggcgccgccgacgccaccgagcagtcccagacccgcacccggtacttccgcggcatgtccggcgacgcgggccgcgccaaggtgcacgtcacgctcacggacgtgaccggcaccgcgaccaccgtcgaggacctgctcccgtaccagggcatggccgccgagacccttacctacaccaaggcgggcggcgacgtcgccgcccgcgagctggccttcccctacagcaggaagaccgcctcccgcgcccgccccggcctccccgccctggaggcgtaccgcacgggcacgacgcgcacggactccatccagcacatcagcggcgaccggacgcgcgccgctcagaaccacaccacatacgacgacgcgtacggcctgcccacccagacctactcgctgacactctcgccgaacgactccggcacccttgtcgccggtgacgagcggtgcaccgtcacgacgtacgtccacaacaccgccgcgcacatcatcggcctccccgaccgcgtccgcgccacgacgggcgactgcgccgccgcgccgaacgccaccaccggccagatcgtctccgacagccgcaccgcgtacgacgcgctcggcgccttcggcacggccccggtcaagggcctgccggtccaggtggacacgatctccggaggcggcacgagctggatcacctcggcgcgcacggagtacgacgcgctgggccgtgcgaccaaggtcaccgacgcggcgggcaactccaccacgaccacgtacagcccggcgaccggccccgcgttcgaggtcaccgtgaccaacgcggctggtcatgccacgaccaccaccctcgaccccggtcgcggctcggcgctgaccgtcaccgaccagaacggccgcaagaccaccagcacgtacgacgaactcggccgggccaccggcgtgtggacgccctcccgcccggtgaaccaggacgcgtccgtgcgcttcgtctaccagatcgaggacagcaaggtcccggcggtgcacactcgggtcctgcgcgacgccggtacgtacgaggagtcgatcgagctctacgacggcttcctccgcccccgtcagacccagcgcgaggcgctgggcggcggccgaatcgtcaccgagaccctctacaacgccaacggctctgcgaaggaagtgcgcgacggctacctggcggagggcgagcccgcgcgggaactgttcgtcccgctctccctcgaccaggtgccgagcgcgacgaggacggcctatgacggcctgggccggcccgtccggacgacgaccctccacaggggagtcccccggcactccgccaccacggcgtacggcggcgactgggaactgagccgcaccggcatgtcgcccgacggaacgacgccgctctctggcagccgcgccgtgaaggcgacgacggacgcgctcggccgcccggcccgcatccagcacttcaccacccagaacgtgtcggccgagagcgtcgacaccacgtacacctacgacccccgcggcccccttgcccaggtcaccgacgcccagcagaacacctggacgtacacgtacgacgcccgtgggcgcaagacgtcctccaccgacccggacgcgggcgccgcctacttcggctacaacgcgctggaccagcaggtctggtcgaaggacaaccagggccgcctgcagtacacgacgtacgacgtcctgggccgccagaccgagctgcgcgacgactccgcgtccggcccgctggtggcgaagtggaccttcgacaccctgccgggcgccaagggccacccggtcgcgtcgacccgctacaacgacggcgccgcgttcaccagcgaggtgaccggttacgacaccgagtaccgtccgaccggcaacaaggtcaccatccccagcaccccgatgaccacgggcctcgccggcacgtacacgtacgccagcacgtacaccccgaccggcaaggtccagtccgtcgacctgcccgcgacgcccggcgggctcgccgcggagaaggtgatcacccgctacgacggcgaggactcgcccaccacgatgtcgggcctggcctggtacacggccgacaccttcctcggcccgtacggggaagtgctgcgcacggcgtcgggcgaggccccgcgccgcgtgtggacgaccaacgtctacgacgaggacacccgccgcctcaccaggaccaccgcgcaccgggagacggctccccacccggtcagcacgaccacctacggctacgacacggtcggcaacatcacgtccatcgccgaccagcagccggcgggtaccgaggagcagtgcttctcgtacgacccgatggggcgcctcgtccacgcctggacggacggcaacagcgccgtctgccccaggacgtccacggcaccgggcgccggcccggcccgcgccgacgtctcggccggtgtcgacggcggcggatactggcactcgtacgcgttcgacgcgatcggcaaccggacgaagctgaccgtccacgaccgcaccgacgcggccctggacgacacgtacacctacacctacggcaagaccctgccgggtaacccgcagccggtccagccgcacaccctcacccaggtcgacgcggtgctcaacgagcccggatcgagagtcgaaccgcgctccacatacgcctacgacacctccggcaacaccacccagcgcgtcatcggcggcgacacccagaccctggcctgggaccgccgcaacaagctgacgtccgtcgacacgaacaacgacggcacaccggacgtgaagtacctgtacgacgcgtcgggcaaccgcctggtcgaggacgacggcaccacgcgcaccctcttcctcggcgaggccgagatcgtcgtcaacacggccggccaggccgtggacgcgcgccgctactacagcagccccggcgccccgacgacgatccgcacgaccggcggcaagaccacgggccacaagctgaccgtcatgctgtcggaccaccacagcacggcgacgaccgcggtcgagctgaccgacacccagccggtcacccgccgccgcttcgacccgtacggcaacccccgcggcaccgagccgaccacctggcccgaccgccgcacctacctgggcgtcggcatcgacgaccccgccacgggcctgacccacatcggcgcccgcgaatacgacgcatcgacgggccgcttcatctccgtcgatccggtcatggacctcacggacccgctccagatgaacgggtacacctacgccaacgcggacccgatcaacaacagcgaccccaccggactgttgctcgacgcccgaggcggcggcactcagaagtgcgtgggaacctgcgtcaaggacgtcacgaaccgaaagggaattccgctcccgcctggcgaggagtggaagcatgaaggggaggcgcaaaccgatttcaacggtgacggcttcatcaccgtcttcccgaccgtgaatgttccggcgaagtggaagaaggcgaagaagtacacggaggctttctacaaggcggttgatactgcttgcttctatggacgcgaaagctgtgcggatccggagtacccttcgcgggcgcatagcatcaacaactggaagggaaaggcatgcaaagccgtagggggaaaatgccctgagaggttgtcgtggggggagggtccggcgttcgctggtggcttcgcgatagcagcggaagagtatgcggggagagggggctaccggggcggtggggcgaggagggggtcgccctgtaagtgcttccttgccggcaccgaggtgctcatggcggatggcagcactaaaagtatcgaggacatcaagctcggtgacgaagtggttgcgactgatccggtaaccggtgaggccggtgcgcaccctgtctcggcgctgatcgccaccgagaacgacaagcgtttcaacgagctggtcattatcaccagcgagggtgtagagcgtcttaccgcaacgcatgagcaccccttctggtcgccatccgaaggggagtggttggaggcgggtgagctgcgcactggcatgacgctgcgctccgactctggcgaaactctcgtagtcgcaggaaaccgcgccttcacccagcgagcccggacctacaacctcacggttgcagacctccacacgtactatgtgctggcgggccagactccggtactggttcacaatgcaaactgtggacctcacctgaaggacctgcaaaaggactacccccggcgcactgtgggcatccttgacgtcggaactgatcagctcccgatgattagcggcccaggtggccagtcgggacttctcaagaacctcccaggtcgtacgaaggccaacggggagcacgtggagactcacgcagcagcgttcttgcgtatgaacccgggtgtcagaaaggccgtgctctacatcgactacccgacggggacctgcggaacatgtagaagtacattgcctgacatgctgcccgagggtgttcagttgtgggtgatctcgccgcgtaggactgaaaaattcacgggacttcctgactga

SEQ ID NO: 369 - protein, 115 aa, Plantactinospora sp.
MGDRLPAFVDGGDTLGIFSRGGIERDLASGVAGPASSLPKGTPGFNGLVKSHVEGHAAALMRQNGIPNAELYINRVPCGSGNGCAAMLPHMLPEGATLRVYGPNGYDRTFTGLPD

SEQ ID NO: 370 - DNA, 348 bp, Plantactinospora sp.
  source 1..348: mol_type = genomic DNA
ctgggtgaccggctccctgccttcgtggacggtggagacacgttgggcatcttttctcgcggaggtattgagcgggacctcgccagcggagttgcgggtcctgcaagtagccttcctaaaggcacgcctggcttcaatggtcttgtaaagagtcatgttgaagggcatgcggctgcgctaatgagacaaaatggaattccgaacgctgagctgtatatcaacagagtgccgtgcggttcaggtaatggctgcgcagcgatgttgccgcatatgcttccggaaggtgccaccctccgcgtatatgggccgaacgggtacgatagaaccttcactggacttccggactga

SEQ ID NO: 371 - protein, 2427 aa, unidentified
  source 1..2427: note = Kitasatospora setae
MAAVPSAEALAAKRARDTIWTPPNTPLGSQTKSVDGENLVPGRLPGPLEPEPADWTPGGPASVPAPGSADVTLGFDSAEAAAARKATGGAAPASDGAALRAGSLPVVIGAAKDAKSGAHRIRVELVDQAKSRAAHLDSPLIALTDTEPDTPPSGRTTKVSLDLKGIGAQTWADRARLVALPACALETPDRPECQQQTPVQSSVDLRSGLLTAEVILPAATEGTAPPTKSSLGSGTASGVVQAGLTTAAPAKAAPTVLAATAGASGSGGSFSATSLSPSAAWGAGSNVGNFTYSYPIQTPPSLGGTAPSVGLGYDSSAVDGKTSAQNSQSSWLGEGWGYEAGFIERGYKSCNTAGIANSSDMCWGGQNATLSLAGHSGTLVRDDTTGVWHLQSDDGTKIEQLTGAPNGLQNGEHWRITTTDGTQFYFGRNHLPGGDGTDPASNSAFKEPVYSPKSGDPCYNSSTATGSWCTMGWRWNLDYAVDVHGNLITYTYAQETNYYSRGAGQNSGSGTLTDYTRAGYLTQIAYGQRLSEQVTAKGAAKAAALITFTAAERCVPSGSITCTEAQRTTANASYWPDTPLDQVCASTGTCTRAGPTFFTTKRLASLTTQVLVSGAYRTVDTWTLTHSFKDPGDGNAKSLWLDSIQRTGTNGQTAVTMPPVTFTAVMKPNRVDGDLTLKDGTKVTVTPFNRPRLQQVTTETGGQINVVYTTSSDAAHPACSRLAGTMPAAADGNTLACAPVKWYLPGSSSPDPVDDWFNKYLISAVTEQDAISGTTLIKATNYTYNGDAAWHRNDAEFTDAKTRTWDGFRGYQSVTSTTGSAYPGEAPRTQQTATYLRGMDGDVKADGSTRSVQVANPLGGPALTDSPWLAGSSFATQTYDQAGGTVISANGSVAGGQQVTATHAQSGGMPALVARYPASQVTTTSKSKLSDGTWRTNTTVSTSDPAHANRPLSSDDKGDGTPGAELCSTNGYATGTNPMMLNILAERTVTKGACGTPVTSANTVSSARTLYDGKPYGQAGDLAESTSALTLDHYDTGGNPVYVHTAASTFDAYGRLTSVSEANGATYDAAGNQLTAPNLTPATTRTAYTPATGAIATTVTQTTPTGWTTTLTQDPGRAEALVSTDANGRATTQQYDGLGRLTAAWSPERATNLTPSQKFSYAVNGTTGPSVVTSQWLKEAGGYAYKNELYDGLGRLRQVQRTSDTYSGRLITDTVYDSHGWPVKTASPYYEKTTAPNSTVYLPQDSQVPAQTWVTFDGIGRTTRSAFVSYGQQQWATTTAYPGADRTDVTPPNGKYPTSTFTDGRNQVSALWQYRTATPTGNPADATVTTYTYDAANRPATRKDAAGNTWSYGYDLRGRQTTVTDPDTGTTTTAYDVNSRAVSTTDGKGNTLVVSYDLIGRKTGLYQGSIAPANQLAGWTYDTLPGGKGKPTSSTRYVGGAGGSAYTQAVTGYDAGYRPTGTSVTIPASEGKLAGTYTTGLTYNPVLGTLKQTDLPAIGAAPAESVMYTYNISGVLQKSYSDTYYVYDVQYDAFGRPVRTTTGDAGTQVVSTQLDKTDYTYNQAGDVTSVTDVQNGTATDAQCFTYDHLGRLTQAWTDTAGSTSTTSGTWTDTSGTVHNSGSSQSVPALGACANANGPASTGSPAKLSVGGPSPYWQSYGYDSTGNRTTLVQHDTTGNTTKDTTTTQTFGPAGSVNTATGAPNTGGGTGGPHALLTSSTTGPTGTQVTSYQYDQLGNTTAVTETSGTTTLAWNGEDKLASVTKTGQAQATSYLYDADGNQLIRRNPGKTTLNLGSDEVTLDTAANSLTDTRYYSAPGGISIARTTGPTGASALAYQASDPHGTANVQINVDAAQTTTRRPTDPFGNPRGTQPAPNTWAGDKGFVGGTKDDTTGLTNLGAREYQPTTGRFLNPDPLLDAGNPQQWNGYAYSDNDPVNSSDPSGLITNALADGDTYVARPAAFCVTMSCVEQTSGPGFWEDKRVGDAVFAAVVQATTQSNGNGSSQTKKEKGIWGQAWDWTKKNGGAILGALVEGAVFSTCFIGAGFAAPATGGITVIAGAAACGAVAGEAGALTTNILTPDADHSVDGITNDMVVGEITGAAVSAASEGASSLAKPAVRKLLGMEAEEGLEAAGRAATGPCNSFPAGVTVLLADGTTKPIEQIAQGDQVTATDPQTGTTQAEPVTDTIIGHDDTEFTDLTLTNDADPRAPPSEITSTTHHPYWNATTSRWTDAGDLKPGDHVRTPDGTELTVNTVYSYTTQPRTARNLTVADLHTYYVLAGNTPVLVHNTGPGCGEPGFVSDAANSLSGRRITTGQIFDASGNPIGPEITSGGGSLADRAQSYLADSPNIRNLPAKARYASADHVEAQYAVWMRENGVTDASVVINQNYVCGLPLGCQAAVPAILPRGSTMTVWYPGSGSPIVLRGVG

SEQ ID NO: 372 - DNA, 7323 bp, unidentified
  source 1..7323: mol_type = genomic DNA; note = Kitasatospora setae
gtgctggggacagcggccgcgctcgcggtcatgatgtccatggcggcggtgccgtccgccgaggcactggccgcgaagcgggcacgcgacaccatctggacgccgcccaacaccccgctgggcagccagaccaagtccgtcgacggcgagaacctcgtcccgggccgcctgcccggccccctggagccggaaccggccgactggacacccggcggaccggcatccgtgcccgctccgggcagcgcggacgtcaccctcggcttcgactccgcggaggccgccgccgcccgcaaggccaccggcggcgccgcccccgcctccgacggcgcggccctccgcgcgggctccctccccgtcgtcatcggcgcggcgaaggacgccaagagcggcgcccaccggatccgcgtcgagctcgtggaccaggccaagagccgtgccgcacacctcgacagcccgctgatcgcactcaccgacaccgagccggacaccccgccctccggtcggaccacgaaggtgtccctcgacctgaagggcatcggcgcccagacctgggcggaccgcgcgcgactcgtcgccctgcccgcctgcgccctggagacgcccgacaggcccgagtgccagcagcagacccccgtgcagagctccgtcgacctgcgctccggactgctgacggccgaggtcattctgcccgccgccaccgagggcaccgccccgcccaccaagagctccctcggctcgggcaccgcctccggcgtcgtccaggccggcctcaccacggcggcgcccgccaaggccgcgcccacggtgctcgccgcgaccgccggcgcgtccggctcgggcggcagcttctcggcgacctcgctgtcgccctccgcggcctggggcgccggctccaacgtcggcaacttcacctactcgtacccgatccagacgcctccctcgctcggcgggaccgccccctccgtgggcctcgggtacgactcgtccgccgtcgacgggaagacctccgcgcagaactcccagtcctcctggctcggcgagggctggggctacgaggccgggttcatcgagcgcggctacaagtcctgcaacacggccggcatcgcgaactcctcggacatgtgctggggcgggcagaacgccaccctctcgctggccggccactccggcaccctggtgcgcgacgacaccaccggcgtctggcacctgcagagcgacgacggcacgaagatcgaacagctcaccggcgcgcccaacggcctgcagaacggcgagcactggcggatcaccacgaccgacggcacgcagttctacttcggccgcaaccacctgcccggcggcgacggcaccgacccggcgagcaactccgccttcaaggaaccggtgtactcgcccaagagcggcgacccctgctacaactcctccaccgccaccggctcctggtgcacgatgggctggcgctggaacctcgactacgccgtcgacgtccacggcaacctgatcacctacacctacgcccaggagaccaactactacagccgaggcgccggccagaacagcggcagcggcaccctgaccgactacacccgcgccggctacctcacccagatcgcctacggccagcgcctgagcgagcaggtcaccgccaagggcgcggccaaggccgctgccctcatcaccttcaccgccgcggaacgctgcgtcccgtccggctcgatcacctgcaccgaggcacagcgcacgaccgcgaacgcctcgtactggccggacaccccgctcgaccaggtctgcgcctccaccggcacctgcacccgggccggcccgacgttcttcaccaccaagcgcctcgcctccctcaccacccaggtcctggtctccggcgcctaccgcaccgtcgacacctggacgctcacccattccttcaaggacccgggcgacggcaacgccaagtcgctgtggctcgactcgatccagcgcaccggcaccaacgggcagaccgcggtcaccatgccgcccgtcaccttcacggcggtgatgaagccgaaccgggtggacggggacctcaccctcaaggacggcaccaaggtcaccgtcaccccgttcaaccggccccgcctccagcaggtcaccacggagaccggcggccagatcaacgtcgtctacaccacctcctccgacgccgcgcaccccgcctgctcgcgcctggccggcaccatgcccgccgcggcggacggcaacaccctcgcctgcgcccccgtcaagtggtacctgcccggatccagctccccggacccggtcgacgactggttcaacaagtacctgatcagcgccgtcaccgaacaggacgcgatcagcggcaccaccctgatcaaggccaccaactacacctacaacggcgacgccgcctggcaccgcaacgacgccgagttcaccgacgccaagacccgcacctgggacggcttccgcggctaccagtccgtcaccagcaccaccggcagcgcctacccgggcgaggcccccaggacccagcagaccgcgacctacctgcgcggcatggacggcgacgtcaaggccgacggctccacccgcagcgtccaggtcgccaacccgctcggcggcccggccctcaccgacagcccgtggctggccggctccagcttcgccacccagacctacgaccaggccggcggcaccgtcatctccgccaacggctccgtcgccggcggccagcaggtcaccgccacccacgcccagagcggcggcatgccggccctggtcgcccgctaccccgcctcccaggtcaccaccacctccaagtccaagctctccgacgggacctggcgcaccaacaccaccgtcagcaccagcgaccccgcgcacgccaaccgccccctcagcagcgacgacaagggcgacggcacccccggcgccgaactgtgcagcaccaacggctacgccaccggcaccaacccgatgatgctgaacatcctcgccgagcggacggtcaccaagggcgcctgcggcacccccgtgacctcggccaacaccgtctcctccgcccgcaccctctacgacggcaagccctacggccaggccggcgacctcgccgagtccaccagcgccctgaccctggaccactacgacaccggcggcaaccccgtctacgtccacaccgccgcctccaccttcgacgcctacggccggcttaccagcgtcagcgaggccaacggcgccacctacgacgccgcgggcaaccagctcaccgcgcccaacctcacccccgccaccacccgcaccgcctacaccccggccaccggcgccatcgccaccaccgtcacccagaccacgcccaccggctggaccaccaccctcacccaggacccgggccgcgccgaagctctggtctccaccgacgccaacggccgcgccaccacccagcagtacgacggcctcggccgcctgaccgccgcctggtcaccggagcgcgcgaccaacctcacccccagccagaagttctcctacgcggtcaacggcaccaccggcccctccgtcgtcacctcccagtggctcaaggaagccggcggctacgcgtacaagaacgagctgtacgacggcctcggccgcctgcgccaggtccagcgcaccagcgacacctactccgggcggctgatcaccgacaccgtctacgactcgcacggctggcccgtcaagaccgccagcccgtactacgagaagaccaccgcgcccaacagcaccgtctacctgccgcaggactcccaggtgcccgcccagacctgggtcaccttcgacggcatcggccggaccacccgctccgcgttcgtctcctacggacagcagcagtgggccaccaccaccgcctaccccggcgccgaccgcaccgacgtcaccccgcccaacggcaaatacccgaccagcaccttcaccgacggccgcaaccaggtcagcgccctgtggcagtaccgcaccgccacccccaccggcaacccggccgacgcgaccgtcaccacctacacctacgacgccgccaaccggcccgccacccgcaaggacgccgccgggaacacctggagctacggctacgacctgcgcggccgccagaccaccgtcaccgaccccgacaccggcaccaccaccaccgcctacgacgtcaactcgcgcgccgtctccaccaccgacggcaagggcaacaccctcgtcgtcagctacgacctgatcggccgcaagaccggcctctaccagggcagcatcgccccggccaaccagctcgccggctggacgtacgacaccctgccgggcggaaagggcaagcccacctcctccacccgctacgtcgggggcgccggcggctcggcctacacccaggccgtcaccggctacgacgccggctaccggcccaccggcacctcggtgacgatccccgccagcgaaggcaagctcgccggtacctacaccaccggcctgacgtacaacccggtcctcggcacgctcaagcagaccgacctgccggccatcggcgcggcgcccgccgagagcgtcatgtacacctacaacatctccggcgtcctgcagaagtcctacagcgacacctactacgtctacgacgtgcagtacgacgccttcggccgcccggtccgcacgaccaccggcgacgccggaacccaggtcgtctccacccagctcgacaagaccgactacacctacaaccaggccggcgacgtcacctcggtcaccgacgtccagaacggcaccgccaccgacgcccagtgcttcacctacgaccacctcgggcgcctcacccaggcctggaccgacaccgcgggctccaccagcaccaccagcggcacctggaccgacacctccggcaccgtccacaacagcggctcctcccagtccgtccccgcactcggcgcctgcgccaacgccaacggccccgccagcaccggcagccccgccaagctctccgtcggcggcccctccccgtactggcagagctacggctacgacagcaccggcaaccgcaccaccctcgtccagcacgacaccaccggcaacaccaccaaggacaccaccaccacccagaccttcggccccgccggatcggtcaacaccgccaccggcgcccccaacaccggcggcggcaccggcggcccgcacgccctgctcaccagcagcaccaccggacccaccgggacccaggtcaccagctaccagtacgaccagctcggcaacaccaccgcggtcaccgagacgtccggaaccaccaccctcgcctggaacggcgaggacaagctcgcctccgtcaccaagaccggccaggcccaggccaccagctacctctacgacgccgacggcaaccagctcatccgccgcaaccccggcaagaccaccctcaacctcggcagcgacgaggtcaccctcgacaccgccgccaactccctcaccgacacccgctactacagcgcccccggcggcatcagcatcgcccgcaccaccggacccaccggcgcaagcgccctcgcctaccaggcctccgacccccacggcaccgccaacgtccagatcaacgtcgacgccgcccagaccaccacccgccgccccaccgaccccttcggcaacccccgcggcacccagcccgcccccaacacctgggccggcgacaagggcttcgtcggcggcaccaaggacgacaccaccggactcaccaacctcggcgcccgcgaataccaacccaccaccggccgcttcctcaaccccgacccactcctcgacgccggcaacccccagcagtggaacggctacgcctacagcgacaacgaccccgtcaacagctccgaccccagcggactcatcaccaacgccctggccgacggcgacacctacgtcgcccgccccgccgccttctgcgtcaccatgtcgtgcgtcgagcagaccagcggccccggtttctgggaggacaagcgcgtcggtgacgccgtcttcgccgccgtcgtccaggccaccacgcagagcaacggcaacgggtcatcccagaccaagaaagagaagggcatctggggccaggcctgggactggaccaagaagaacggcggcgccatcctcggagcgctggtagagggagcggtcttcagcacatgcttcatcggagctggattcgccgcacctgcaacgggaggaatcaccgtcatcgccggtgctgcggcctgcggggctgtggccggcgaggcaggggcactgaccaccaatatcctcaccccagatgccgaccactccgtcgacggcatcaccaacgacatggtcgttggtgaaatcaccggggcggctgtcagcgcagcgagcgagggcgcaagctccctcgccaagccggcggtccgcaaactcctgggcatggaagccgaggaaggactcgaggcagcaggccgcgccgccaccggaccttgcaacagtttcccggccggcgtcaccgtcctcctcgccgacggcaccaccaagcccatcgaacagatcgcccagggcgaccaggtaaccgccaccgacccgcagacaggcaccacccaggcagaacccgtcaccgacacgatcatcggccacgacgacacggaattcaccgacctcaccctcaccaacgacgcagacccccgcgccccgcccagcgagatcacctccaccacccaccacccctactggaacgccaccaccagccgctggaccgatgccggcgacctcaagcccggcgaccacgtccgcacccccgacggcaccgaactgaccgtcaacaccgtctacagctacaccacacaaccccggaccgcgcgcaacctcaccgtcgcagacctccacacgtactatgtgctcgctggaaatacgccggtcctagtgcataacaccggcccgggatgtggtgagccgggattcgttagtgacgctgctaattctctctcgggcaggcgcatcaccacgggacaaatatttgatgcgagcgggaatccgatcgggcctgagatcacgagcggcggcggcagtctggcagatagggcgcagagttatcttgccgactcccctaatattcgaaatctgcccgctaaggcgagatatgcgtcggctgaccacgttgaggcgcaatatgcagtgtggatgcgagaaaatggagtgaccgacgccagtgtggtcatcaatcaaaactatgtatgtgggctgcccctaggctgccaggcggcggtgcccgctatcctccctcgcggctcgaccatgacggtatggtatccagggtcaggaagtcccatcgtattgcggggagtgggttaa

SEQ ID NO: 373 - protein, 2594 aa, Thauera sp.
MRAFRLIACLLAFSAAAAPAAADTSSMLGRLPEASARQLKERLAPRGLASAAALRQYLDASQRELDTAPEADDVPARSQRFAARAGELTALREQARRDLASLEDAAKASGSAEATQRIGRIRGQVDARFDRLEGLFTTWRNAPQGSERRQARRELRAALATLRHAGTPAPAAIPVPTLGPLQPAGEPAANPPAARLPAYAQADDATGDPFTPGGFRLMKVAALPPAVAAEAATDCSATSADLADDGKDVRLTQPIRDLAASLDYSPARILRWTQQNVAFEPYWGALKGAEGVLQTRAGNSTDQASLLIALLRASNIPARYVRGTVQLNDTAAQDDAGGRAQRWLGTKRYRASAAVLAGGGTSAGLQSIDGTVRGIRFSHVWVQACVPHGAYRGARAEAGGYRWLALDAAVKDHDYQQGIAVDVPLTDAAFYTPYLAARSDQLPHEHFAQKVAEAARATDANAALADVPYAGTPRPLRYDVLPGSLPYEVEAFTNWPGLGSSETASLPDAHRHTFTVTVRNGATTLASAALPYPQNAFKRVTLSYQPTAASQAAWNAWTGDLPAAADGSIQVVPQIKADGTVLAAGAPANALPLAGVHNVILKVSQGERSGAACINDSGNPADPKDTDGTCLNKTVYTNIKAGAYHALGLNALHTSNAFLGQRLEALAAGVQAYPVAPTPAAGAGYEATVGELLHLVLQDYLHQTEQADQRNAALRGFKSVGPYDLGLTASDLETDYLFDIPVAIKPAGVFVDFKGGLYGFVKLDTTAETAAARAAENVDLAKLSIYSGSALEHHVWQQALRTDAVSTVRGLQFAAEQGIPLVTFTAANIGQYDSLMQMSGATSMAAYKSAIQNAVKGSDNGNHGVVTVPRAQIAYADPVDPASKWTGAVYMSQNPVTGEYGAIINGTIAGGFPLLNSTPFSNLYNFDSFVPNTLLGTNGGAGAVQTLPGGTQGESSWITKAGDPVNMLTGNYTLQARDFTIKGRGGLPIVLERWFNAQNATDGPFGFGWTHSFNHQLRFYGIESGQSKVGWVDGTGAQRFYAVAAAGSIAPGTTLAAQAGVFTTLSRLADGRFQVRETNGLTYSFESLTSPTTPPAAGSEPRARLLAIADRHGNTLTLNYSGSQLASVSDSLGRTVLSFTWNGNRIGKVKDVSGREVNYAYEDGNGNLTRVTDPLGQATRYSYYTSADGAKLDHALRRHTLPRGNGMEFEYYAGGQVFRHTPFDTSGNLIPESALTFHYNSYRRESWTVDGRGAEERFLFDTHGNVIQQTAANGATHTYAYADPNDPHLRTRMTDPVGRVTQYSYTAEGYLQTLTLPSGAVQAWRDYDAFGQPRRVKDARGNWTLHHYDTAGTRTDSIRVKSGVVPTVGTAPAAANVVSWIKYQGDSVGNLTGVKRLRDWTGATLGNFASGSGPVVTTTFDAARLNVASVGRSGNRNGSQISETSPIFSHDALGRLTGGVDGRWYPVAFDYDVLDRVTRATDATGQPRRYAFDVNGNRIGTELIAGGSRIDSSVAAFDVQDRVAHVLDHAGNRVAYAYDAVGNRVSVESPDGYAIGFDYDLAGRPYSAYDEDGNRVFSAFDVAGRVRAVIDPNGAATLYDYHGDEQDGRLARVEQPAIPGQNAGRAAETDYDAGGLPIRVRQVSAGGEAREGYRFHDELGRVVRSVSAPDDVGQRLQVCYSYDALSNLTQVRAGATTDTTSAACAGSPAVQLTQSWDDFGNLLTRTDALGRVWKFEYDAHGNLVASQTPEQAKVSTRSTYRYDPALHGLLAGRSVPGSGSAGQSVSYARNALGQVIRAETRDGAGNLVVAYDYQYDAAHRVVRIVDSRGGKALDYAWTPAGRLASITLDGHVWRFQYDGVGRLAAIVAPNGATIAMARDAAGRLTERRWPDGAKSAFDWLPEGSLAAIEHSAGGSALAQFAYAYDAWGNRTSATETLAGTSRSLAYGYDALDRLKTVTTDGATETHAFDLFGNRTSKTTGGVTTDYLFDAAHQLTQVQIAGTPTERLAYDDNGNLRKHCVGSPSGSTSDCTGTTVLSLAWNGLDQLIQAARTGLPAESYAYDDAGRRVTKAVGSSATHFAYDGPDILAEYASPAGSPTAVYAHGAGIDEPLLRLTGATSTPAASAHHYAQDGLGSIVAAYGEIGASGPVSAASVSATHSYSAGSYPPAKLIDGETTGSTGFWAGSSGNFAADPAVITLELGAEKSVSRVRLHRVASYLPDYVVKDAEVQVRKPDNSWQTVGTLTNNTSEDSPEIVLTGAPGSALRVLVKGVRNGSLVLMAEVTMSADGGAASVATARYDAWGNVTQASGSIPAFGYTGREPDATGLVYYRARYYHPALGRFASRDPLGLAAGINPYAYAGGNPILYNDPDGLLAQLAWNTAASYWGQPIVQETVATIRNGAAVAAGNFVPDTVNGATGWFEQFLHQESGSFGRMDSWVDVRNPVAQDVAQDLRGVAAVGLMMTPLRYGRASNASFNPPVANLPLNTGGKTSGMLHIPGQESLSLTSGIAGPSQVVRGQGLPGFNGNQLTHVEGHAAAYMRTHKVSEAVLDINKAPCTAGSGGGCNGLLPRMLPEGAHLTIRHPNGVQVYIGTPD

SEQ ID NO: 374 - DNA, 7785 bp, Thauera sp.
  source 1..7785: mol_type = genomic DNA
atgcgtgccttccgcctgatcgcctgccttctcgccttttcggcggcagccgcacctgctgcggctgacacgtcgtcgatgctggggcgtctgcctgaagcaagcgcccgccagctcaaggagcggttggcgccgcgtggccttgcctccgctgccgccttgcgccagtacctggacgcctcgcaacgcgagctggacaccgcaccggaagcggacgacgtacccgcccgcagccaacgctttgccgcaagggcgggcgaactcaccgcgctgcgcgaacaggcgcgccgggatctcgccagtctggaggacgccgcgaaggcgagcggctcggccgaggcgacgcagcgcatcggtcgaatccgcgggcaggtggacgcacgcttcgaccggctcgaagggctttttaccacttggcgcaatgcgccccagggcagcgaacgccgccaggcccgccgcgaactgcgtgccgcgctcgccacgctccgccatgccggcaccccggctccggctgcgattcctgttcctaccctcggccccctgcaaccggccggcgagccggctgccaacccaccggccgcgcgcttgccagcctatgcgcaagcggatgacgcgactggcgacccctttacccccggtggcttccggctgatgaaggtcgccgcactgccgccggcggtcgcggccgaggcggcaacggactgctccgccaccagcgccgacctggccgacgacggcaaggacgtgcgcctgacccagccgatccgcgacctcgcggcatcgctcgactactcaccggcacgcatcctgcgctggacgcagcagaacgtcgccttcgaaccctactggggggcactcaagggggcggaaggcgtgctgcagacgcgcgccggcaacagcaccgaccaggccagcctgctgatcgcactcttgcgggcctccaacattcccgcccgctacgtacgcggcaccgtgcagctcaacgacactgccgcgcaggacgacgcaggcgggcgggcgcagcgctggctgggcaccaagcgctaccgtgcatcggccgcggtactcgccggcggcggaacttccgccggcctgcagtcgatcgacggcaccgtccgcggcatccgcttcagccatgtctgggtccaggcctgcgttccccatggcgcttaccgcggtgcccgcgcggaagccggcggctatcgctggctggcgctggacgcggcggtgaaggaccatgactaccagcagggcatcgcggtcgatgtgccgctcaccgatgccgcgttctacacgccctatctggcggcgcgcagcgaccagttgccgcacgagcatttcgcacagaaggtggcggaggcggcgcgtgcgaccgacgccaatgcggcgctggccgacgtgccctacgccggtacgccgcggccgctgcgctacgacgtgctgcccggttcgctgccctacgaggtcgaagccttcaccaactggcccggcctcggttcgtccgaaaccgcaagcctgccggacgcacaccgccacaccttcaccgtgacggtcaggaacggcgccaccacgttggcgagcgccgcgctgccctatccgcagaacgccttcaagcgcgtcacgctgtcctatcagccgactgccgcctcgcaggcggcctggaacgcctggacgggcgatctgcccgccgcggccgacggcagcatccaggtcgtgccgcagatcaaggccgacggtaccgtgctcgccgcaggtgcgcccgccaacgcgctgccgctcgccggcgtgcacaacgtcatcctcaaggtctcgcagggcgagcgcagcggtgccgcgtgcatcaacgacagcggcaaccccgccgacccgaaggacaccgacggcacctgcctcaacaagaccgtctacaccaacatcaaggccggcgcctaccacgccctgggcctgaatgcgctgcacacctcgaatgccttcctcggccagcggctcgaagcgctggcggccggcgtgcaggcctatcccgtcgcgcccacgccggccgcgggtgccggctacgaggccacggtcggtgaattgctgcatctggtgctgcaggactacctgcaccagaccgagcaggccgaccagcgcaacgccgcgttgcgcggcttcaagagcgtggggccgtacgacctcgggctgaccgcgtccgacctcgaaaccgactacctcttcgacatcccggtcgcgatcaagccggccggcgtgttcgtggacttcaagggcggcctctacggtttcgtcaaactcgataccacggccgagacggccgcggcacgcgccgccgaaaacgtggatctggccaagctctcgatctactccggctccgcgctcgaacaccacgtctggcagcaggcgctgcgcaccgatgcggtgtccaccgtgcgtgggctgcagttcgccgccgagcagggcattccgctcgtcaccttcaccgcggccaacatcggccagtacgacagcctcatgcagatgagcggcgccaccagcatggccgcttacaagagcgcgatccagaacgcggtgaagggctcggacaacggcaaccacggcgtcgtcaccgtgccgcgcgcccagatcgcctacgccgaccccgtcgatccggcgagcaaatggaccggcgcggtctacatgtctcagaaccccgtcaccggagagtacggggcgatcatcaacggcaccatcgccggcggcttcccgctgctcaacagcacgcccttcagcaatctctacaacttcgattccttcgtgcccaacaccctccttggcacgaacgggggtgccggtgcggtgcagaccctgcccggcggcacccagggcgagagttcctggatcaccaaggccggcgacccggtgaacatgctcaccggcaactacacgctgcaggcacgcgacttcaccatcaagggccggggcggactgccgatcgtgctggagcgctggttcaacgcgcagaacgccaccgacgggccgttcggcttcggctggacgcacagcttcaaccatcagttgcgtttctacggcatcgagagcggccagtccaaggtcggctgggtggacggcactggcgcccagcgcttctacgccgtggccgccgccggcagcattgcgccgggcacgacgctggccgcgcaggccggggtgttcacgacgctgtcgcgtctggccgacggccgcttccaggtgcgcgagaccaacggcctcacctacagcttcgaatcgctcacgagcccgaccaccccgccggccgcgggcagcgaaccgcgcgcaagactgctggccatcgccgaccgccacggcaacaccctgacgctcaactacagcggcagccagcttgcctcggtgagcgacagcctcggccgcacggtgctcagcttcacctggaacggcaaccgcatcggcaaggtgaaggacgtcagcggacgggaagtgaactacgcctacgaggacggcaacggcaacctcacgcgcgtcaccgatccgctgggtcaagccacgcgctacagctactacaccagtgccgacggtgccaagctcgaccacgccctgcgccgccacaccctgccgcgcggcaacggcatggagttcgagtactacgccggtggccaggtcttccgccacacgccgttcgacaccagcggcaacctcattcccgaatcggcgctgaccttccactacaacagttatcggcgcgagagctggacggtcgatggccgcggtgccgaggagcgcttcctgttcgacacgcacggcaacgtgatccagcagaccgccgccaacggtgccacccacacctacgcgtacgccgacccgaacgatccgcatctgcgcacgcgcatgacagacccggtcggccgcgtcacccagtacagctataccgccgaaggctatctgcagaccctgacgctgccgtcgggcgccgtgcaggcgtggcgcgactacgacgccttcggccagccccgccgcgtcaaggacgcgcgcggcaactggacgctccaccactacgacaccgccgggacacggaccgactccatccgggtcaaatcgggcgtggtccccaccgtcggcaccgcgcctgccgcggccaacgtcgtttcctggatcaagtaccagggcgacagcgtgggcaacctcaccggcgtcaagcgcctgcgcgactggacgggcgcgaccctgggcaatttcgccagcggcagcggccccgtcgtcaccaccaccttcgatgcggccaggctcaacgtcgccagcgtcggccgtagcggcaaccgcaacggcagccagatcagcgagaccagcccgatcttctcccacgacgcgctggggcgcctcaccggcggggtggacgggcgctggtatccggtcgccttcgattacgacgtgctcgaccgcgtcacccgcgccaccgacgccacgggccagccgcgccgctacgcgttcgacgtcaacggcaaccgcatcggtacggagctgattgccggcggcagccgtatcgattcctcggtggccgccttcgacgtgcaggaccgcgtcgcccacgtcctcgatcacgccggcaaccgcgtggcctacgcctacgatgcggtgggcaaccgggtgagcgtggaaagccccgacggctacgccatcggcttcgactacgacctcgccggacggccctattcggcctacgacgaagacggcaaccgcgtcttctccgccttcgacgtggccgggcgcgtgcgagcggtcatcgaccccaacggcgccgcgacgctctacgactatcacggcgacgagcaggacgggcgtctcgcgcgcgtggagcagcccgccatcccgggccagaacgcgggccgcgccgccgagaccgactacgatgcgggtgggttgcccatccgcgtgcgccaggtctcggccggcggcgaagcgcgcgaaggctaccgtttccacgacgagcttggccgcgtggtgcgcagcgtctccgcgccggacgacgtcggccagcggctgcaggtctgctacagctacgatgcactctcgaacctcacccaggtgcgcgccggcgccaccaccgacaccaccagtgccgcctgcgccggcagccccgcggtgcagctcacccagagctgggacgactttggcaacctgctgacgcgcaccgacgcgctgggccgggtgtggaagttcgagtacgacgcccacggcaacctcgtcgccagccagacgcccgagcaggccaaggtctcgacgcgcagcacctaccgctacgatccggcgctgcacggcttgctggccgggcgcagcgtgccgggcagcggcagtgcgggccagagcgtgagctatgcgcgcaacgcgctcggccaggtcatccgcgccgagacgcgcgacggcgcgggcaacctcgtcgtcgcctacgactaccagtacgacgccgcccaccgtgtggtgcgcatcgtcgacagccgcggcggcaaggcgctcgactacgcctggacgcccgccgggcggctggcgagcattaccctggacggccatgtctggcgcttccagtacgacggcgtcggccggctcgccgcgatcgtcgcgcccaacggcgccaccatagcgatggcacgcgatgccgccgggcggctcaccgagcggcgctggcccgacggcgcgaagagcgccttcgactggctgcccgaaggcagcctcgccgccatcgagcacagcgcgggcggcagcgcgctcgcacagttcgcctatgcctacgatgcctggggcaaccgcacgagcgccaccgagaccctcgcgggcaccagccgcagcctcgcctacggctacgacgcgctcgaccgcctgaagaccgtcaccaccgacggtgcgaccgaaacccatgccttcgatctcttcggcaatcgcaccagcaagaccacgggcggggtgaccaccgactatctcttcgacgcggcgcaccagctcacccaggtgcagatcgccggcacccccaccgagcggctcgcctacgacgacaacggtaatctccgcaagcactgcgtcggcagtccgagtggcagcaccagcgattgcaccggcaccaccgtgctgagcctcgcctggaacggcctcgaccagttgatccaggccgccaggacgggcctgcccgccgagtcctacgcctacgacgatgccgggcggcgtgtcaccaaggcggtgggcagcagcgccacccacttcgcctacgacggtcccgacatcctggccgagtacgccagcccggccggcagccccaccgccgtctatgcccacggtgccggcatcgacgaaccgctgctgcgcctcaccggcgcgacgagcacgccggccgcttccgcgcaccactacgcgcaggacgggctgggcagcatcgtcgcggcctatggcgagatcggcgccagcggtccggtcagtgccgcgagcgtatcggccacccacagttacagcgccggcagctacccgccggcaaagctgatcgacggcgagacgaccggaagcaccgggttctgggctggcagctcgggcaacttcgctgccgatccagccgtgatcacgctggaactgggtgcggagaaaagcgtgagccgcgtgaggctgcaccgggtggccagctacctgcccgactacgtggtcaaggatgccgaggtgcaggtccgaaaaccggacaattcgtggcagacggtcggcacgctgacaaacaacaccagcgaagacagtcccgagatcgtgctcaccggcgcccccggcagcgcgctgcgcgtgctcgtcaagggcgtgcgcaacggcagcctggtgctgatggccgaggtgacgatgagtgcggacggtggcgcggccagcgtggccaccgcccgctacgacgcctggggcaacgtcacgcaggcgagcggcagcatcccggccttcggctacaccggacgcgagcccgatgccacgggcctggtctactaccgcgcccgctactaccaccccgcgctcggccgcttcgccagccgcgacccgctggggctggcggcggggatcaatccctacgcctacgcgggcggcaatcccatcctctacaacgatccggatggcttgctggcgcaactggcgtggaatacggcggccagctactggggacagccgatagttcaagaaacggtcgccacgattcgaaatggggccgcagtggccgctggcaacttcgttccagacacggtcaacggtgcaacaggttggtttgagcagttcctgcaccaagaatcgggctcgttcgggcgcatggactcgtgggtggatgtgcgaaaccccgttgcgcaggacgtagcccaggacctgcgcggtgtcgcagccgttgggttaatgatgacgccgctgcggtatggtcgtgcctccaacgcgtctttcaatccgccagtagccaatcttccgctcaacactggaggaaaaacatctggcatgttgcacattccagggcaagaatcactgtcgctcacgagcggaattgcggggccgtctcaagtcgttagaggtcaaggtttgccaggattcaacggtaatcagttgacccatgtggaaggtcatgctgctgcttacatgcggactcacaaggtctctgaggctgttctggacataaacaaagcaccttgcaccgctggtagtggtggtggatgtaatgggttgcttccccgaatgctgccggagggggctcatttaacaattcgacacccaaatggtgttcaagtttatattggcactcctgactaa

SEQ ID NO: 375 - protein, 2227 aa, Chondromyces crocatus
MSMSASRSQPAFPFVSASSPRPRRRPPFPRALLLLIAVLLVGACGDAGGPLLWSSSSQALWEPSPIPPLPPLLCLGPGDGPSPFPPDLTQGTTTAAGTLPGSFSVTSTGEATYTIPVPTLPGRAGIEPSLAITYDSAQGEGLLGIGFHLQGLSSVDRCPRNVAQDGHIAPVRDAEDDALCLDGQRLVPVDPQPGRAPREYRTFPDSFTRVEADFAESEGWPAERGPKRLRAHGKAGLIYEYGGESSGRVLAQGEAVRSWLLTRLSDRDGNTMAVVYRNDLHAKGYTVEHAPQRITYTRHPTVPASRMVEFTYGPLEAADVRVHYARGMELRRSLSLRSIQMFGPGHVLARELRFGYGHGPATGRLRLEAVRECAGDGTCKPPTRFTWHTAGAAGYTQQQTLVEVPLSERGTLMTMDVSGDGLDDLVTSDMVVEAGTEEPITRWSVALNRSQELTPGFFEAAVTGQEQPHFIDAEPPYQPELGTPLDYDHDGRMDLFLHDVHGQSMTWEVLLSNGDGRFTRRDTGVPRPFTMGMTPAGLRSPDASTHLVDVDGDGMVDLLQCYLSAHEQLWYLHRWTAAAGGFAPHGDRVHALSSYPCHAELHAVDVDADGRVDLVMQELILVGSQVRAGWQYVAFSYELSDGSWTRALTGLRLTPPGDRVFFLDVNGDGLPDAVQSSRDDEQLYTSMNIGAGFAAPVPSLATPTLGAARFVRFASVLDHNADGRQDLLLAMSDGGSESLPAWKVLQATGEVGPGTFEIVHPGLPMGIVLQQDELPTPDHPLTPRVTDVNGDGAQDLLYAFNNQVHVFENVLGQEDLLAAVTDGMNAHAPEDAEYLPNVQIRYDHLIDRARTTEGFEDAPGIPSPEQRTYRPLEQSDEEPCRYPVRCVVGHRRVVSGYVLNNGADRPRTFQVAYRNGRHHRLGRGFLGFGTRIVRDLDTGAGTAEFYDNVTFDGAFQAFPFRGQVQRSWRWSPSLPLDAHSAEPASLELLTTRSYAVVIPTQAGTYFTLSLLEGKSRHQGTFSPGSGKTLEEAVRALEGDLASRMSDTLRTVSDFDLYGNILAEQTQTEGVDLDLSVTRSFDNDPLSWRLGELTRETTCSKAGGETQCRVMHRSYDGRGHVRLERVGGEPFDPEMQLDVWFSRDALGNIHSTRSRDGTGQVRASCTSYDALGLMPYAHRNLEGHQSYTRYDPAVGVLRASVDPNGLVSRWAYDGFGRVTLESLPGRMPTVIRRTWTKDGGAAGNAWNLKIRTASVGGQDETVQLDGLGREVRWWWQALDVGEEQAPRMMQEVAFDARGEHLAWRSLPIVDPAPPGSVQVRETWQYDGMGRVLRHVTPWGAATTHEYIGRDEVITAPGQAVTRIASDPLGRPTAVGDPEGGVSRYTYGPFGGLREVTTPAGAVTLTERDAFGRVRRQVSPDRGVSTAHYDGYGQKISSLDAAGRAVTTRYDTLGRIFRQVDEDGVTEFRWDDAQHGVGQLALVVSPDGHRLRYGFDHLGRPATTTLEIGGESFTSRLSYDLSGRLERIEYPSAPGIGSFAIEREYDPHGRLRALKDAGSGAEFWRATAIDAGNRITGERFGGGTATTLRTFDAARERVSRIETQTAGGPVQQLSYLWNDRRKLVERSDGLHANVERFRYDLLDRLTCAQFGLINAALCERPFTYGPDGNLLQKPGVGAYEYDPAQPHAVVRAGSAFYGYDAVGNQTSRPGATIAYTAFDLPKRIALTSGDTVDFAYDGLQQRVRKTTATQEIASFGEVYERVTDVVTGAVEHRYHVRNDERVVTLVRRSVAQGTRTLHVHVDHLGSIDVLTDGVTGSVAERRSYDAFGAPRHPDWGSGQPPSPHELSSLGFTGHEADLDLGLVNMKGRIYDPKLGRFLTPDPLVPRPLFGQSWNSYSYVLNSPLSLVDPSGFQEQPPATEDGCSQGCTIWVFGPPREPKPPAPPKVVEGNLEDAAGTGSTQAPVDVGTSGVRSGWSPQLPATLQTLGRGDAIARRIMDGVRIGMARMLLESAKLGILGGTSRVYVAYTNLTAAWNGYKESGLPGALDAVNPASQMVQAGVEAYEAAAAEDWEAAGASLFKAGSIGMSILATAVGVGGAITATVGSTAGAAGRAAARAPSLPAYAGGKTSGVLRTTAGDTALLSGYKGPSASMPRGTPGMNGRIKSHVEAHAAAVMREQGMKEGTLYINRVPCSGATGCDAMLPRMLPPDAHLRVVGPNGYDQVFVGLPD

SEQ ID NO: 376 - DNA, 6684 bp, Chondromyces crocatus
  source 1..6684: mol_type = genomic DNA
atgtccatgtcggcctcacggagtcagcccgcattccccttcgtgtcggcctcctctccgcgtccgcgccggcgccctccctttccccgagcgctgctcctcctcatcgccgtgctcctcgtcggcgcatgcggcgacgctggcggcccgcttctctggtcgagcagctcccaggccctctgggaaccctccccgatcccgccgctccccccgctcctgtgcctcggccccggcgacggtccctccccctttccgcctgaccttacgcaggggaccaccaccgcggcggggaccctgccagggagcttttcggtcacgagcacgggcgaggcgacgtacacgatcccggtccccacgctgcctggccgtgccggcatcgagccctcgctggcgatcacctacgacagtgcgcagggtgaagggctgctcgggatcggcttccacttgcagggcctctcgtcggtcgatcgctgcccccggaacgtcgcgcaggatggtcacatcgcgccggtccgggatgccgaggacgacgccttgtgcctcgatgggcagcggctcgtccccgtggacccgcagccagggcgtgcgccgcgggaataccgcacgttcccggacagcttcacgcgcgtcgaggccgacttcgcggagagcgaggggtggccggcggagcgtgggccgaagcggctgcgggcgcatggcaaagcggggctgatctacgaatacggtggagaatcatcgggccgggtgctcgcgcaaggggaggcggtgcggtcctggttgctgacgcggctcagcgaccgggatggcaacacgatggcggtggtctaccggaatgacctccacgcgaagggctacaccgtcgagcacgcgccgcagcggatcacctacaccaggcacccgactgtgccggcctcgcgcatggtggagttcacgtacgggccgctggaggcggcggacgtgcgcgtacactatgcccgcgggatggagctgcgccgctcgctgagcttgcgctcgatccagatgttcgggccgggacacgtgctcgcgagggagctgcgcttcggttacgggcatgggccggcgacgggtcgcttgcgactggaggcggttcgggagtgcgcaggtgacgggacgtgcaagccgccgacacgcttcacctggcacacggccggagcggctggatacacgcagcagcagacactggtggaggtgccgctgtcggagcgcggcacgttgatgacgatggacgtcagcggcgatggcctcgacgacctggtgacgtccgacatggtggtggaggccggcacggaagagccgatcacccgctggtcggtcgcgctcaaccggagccaggagctgacgccggggttcttcgaggcggccgtcactgggcaggagcagccgcatttcatcgacgcagagccgccgtaccagccggagctggggacgccgctcgactacgaccacgatggccggatggacctgtttctgcacgatgtgcacgggcagtcgatgacgtgggaggtgctgctgtcgaatggagatgggcggttcacgcggcgggatacgggggtgccgcggccgttcacgatgggcatgacgccggcgggattgcgcagcccggatgcgtcgacccatctggtggatgttgacggtgacgggatggtggacctgctgcagtgctacctgagcgcgcacgagcagctctggtacttgcaccgctggacggcagcggcggggggcttcgcgccgcacggcgatcgggtgcatgcgctgagctcctacccgtgccacgccgagctgcacgcggtcgatgtcgacgcggatgggcgggtggacctggtgatgcaggagctgatcctcgtcgggagccaggtgcgggcggggtggcagtacgtggcgttctcgtacgagctgtccgatggatcgtggacgcgcgcgctgacggggctgcggctcacgccgcctggggaccgggtgttcttcctcgacgtcaacggcgatgggctgcccgatgcggtgcagagcagccgggacgatgagcagctgtacacgtcgatgaatatcggcgcgggattcgcggcgccggtaccgagcctggcgacgccgacgctcggggctgcgaggttcgttcggtttgcgtcggtgctcgatcacaacgcggatgggcgacaagacctgctgctggccatgagcgatgggggatcggagtcgctgcccgcgtggaaggtgctccaggcgacgggggaggtcggtccggggacgttcgagatcgtccatcccgggctgccgatgggcatcgtgctccagcaggacgagctgcccacgcccgaccatccgctcacgccgcgggtcactgacgtgaatggggatggggcgcaggatctgctctatgcgttcaacaaccaggtccatgtgttcgagaacgtgctcggccaggaggacctgctcgcggccgtgaccgacggcatgaatgcgcacgctccggaggacgccgagtacctgcccaacgtgcagatccggtacgaccacctgatcgatcgtgcgcggacgacggagggcttcgaggatgctccagggatcccgtcacccgagcagcgcacctaccggcctctggagcaaagcgatgaggagccctgccgctatccggtgcggtgcgtggtcgggcatcggcgggtggtgagcggctatgtgctcaacaatggcgcggatcggccgcgcaccttccaggtggcctaccgcaatggccgtcaccatcgcctgggccgagggtttctggggttcgggacgcggatcgtgcgtgacctcgataccggcgcggggacggccgagttctacgacaacgtcacgtttgatggcgccttccaggccttccctttccgagggcaggtacagcgctcgtggcgctggagtccgagcttgccgctggacgcgcatagcgcggagccggcgtccctcgagctgctgacgacgcggagctacgcggtggtgatccccacgcaagcggggacgtacttcaccctctcgctgctggagggcaagagccgtcatcagggcacgttctcaccggggagtgggaaaacgctcgaagaagccgtgcgcgctctggaaggagatctcgcctcgcgaatgagcgacacgctccgcaccgtcagcgacttcgacctctacgggaacatcctcgccgagcaaacgcagacggagggcgtcgacctcgacctctcggtgacgcgcagcttcgacaacgacccgctctcctggcgccttggcgagctgacgcgagagacgacgtgcagcaaagcgggcggtgagacgcagtgccgggtgatgcaccggagctatgacgggcgcggccacgttcgcctggagcgcgtcgggggagagcccttcgacccggagatgcagctcgatgtctggttctcgcgggacgcgctgggcaacatccacagcacccggtcacgtgatgggacggggcaggtgcgcgcgagctgcaccagctacgacgcgctgggcttgatgccttatgcccaccgcaacctggagggccaccagagctatacgcgctacgacccggccgtgggcgtgctgcgggcgtcggtggatcccaacggcctggtgagccgctgggcctacgatggcttcgggcgggtgacgctggagagcctccccgggcgcatgcccaccgtcatccggcggacctggacgaaggacggcggagcggctggcaacgcctggaacctgaagatccgcaccgcctcggtggggggccaggacgagaccgtgcagctcgatggtctcgggcgggaggtgcgctggtggtggcaagcgctcgacgtgggggaagagcaagcgccgcggatgatgcaggaggtcgccttcgatgcgcggggcgagcacctcgcgtggcgctcgctgccgatcgtggatcccgcgccaccaggctcggtgcaggtgcgagagacgtggcaatacgacgggatggggcgggtgctccggcacgtcacgccgtggggggcggcgacgacgcacgagtacatcgggcgggacgaggtcatcaccgcgcctgggcaggccgtcacccgaatcgccagcgatccgctcgggaggcccacggcagtgggtgatcccgaaggtggcgtcagccggtacacctacggtcccttcggggggctgcgcgaggtgaccacgcccgctggtgccgtgacgctgaccgagcgggatgcgtttggccgcgtgcgacggcaggtgagcccggaccggggagtctctactgcgcactacgacggttacgggcagaagatctcatcgctcgacgcggcaggacgcgcggtcacgacccgctacgacacgctgggtcggattttcaggcaggtcgacgaagacggcgtcaccgagttccgttgggatgacgcgcagcatggagtgggtcagctcgcgctggtggtcagccccgatgggcatcggctgcgctacggcttcgaccacctcgggcgaccagcgacgacgacgctggagatcggaggggaaagcttcaccagccggctgtcttatgatctgagcggccggctcgagcggatcgagtacccgagcgcgccggggattggcagcttcgccatcgagcgggagtacgatcctcacgggcggctgcgggcgctgaaggatgcggggtcgggggcggagttctggcgagccaccgcgatcgatgcggggaatcgcatcacgggggagcgcttcggtggggggaccgccaccacgctccgcacgttcgacgcggcacgggagcgggtgagtcggatcgagacgcagacggcaggtgggcccgtccagcagctctcctacctctggaacgatcgccgcaagctcgtcgagcgctccgatggcctccacgccaacgtcgagcgctttcgttacgacctgctggaccggctgacgtgcgcgcagttcgggctgatcaatgctgccctctgcgagcgaccgttcacctacggacccgacggcaacctgctccagaagcccggcgtcggtgcctacgagtacgaccccgcgcagccccacgccgtcgtccgagctggtagcgcgttctacggctacgacgccgtcggcaaccagacctcacgacccggcgcgaccatcgcctacaccgcgttcgacctaccgaagcgaatcgcgctcaccagcggcgacaccgtcgacttcgcgtacgacggcctccagcagcgggtgcgcaagaccacggcgacgcaggagatcgcctccttcggcgaggtgtacgagcgcgtgaccgatgtcgtcacgggagccgtcgagcatcgctaccacgtgcgcaacgacgagcgcgtcgtcacgctggtgcggcgctcggtcgcgcaaggcacgcgcacgctgcatgtccatgtcgaccacctcgggtcgatcgatgtgctcaccgacggtgtgaccggcagcgtcgccgagcgccgcagctacgatgccttcggcgcaccgcgccatcccgactggggttcgggtcagcctccgtcaccccacgagctgtcgtcgcttggcttcaccgggcacgaggccgacctcgacctcggcctcgtgaacatgaaggggcgcatctacgaccccaagctcggacggttcctcacgcccgatccgctcgtgccgcggcctctcttcgggcagagctggaatagctattcgtacgtgctaaacagcccgctgtcgctggtcgatcccagtgggtttcaagagcagccacctgcgacagaggacggatgctcgcagggctgcaccatctgggtgttcggtcctccccgcgagccgaagccacctgcgccgcccaaggtcgtcgagggcaacctggaggacgccgctggcactggttcgacccaggcgccggtcgatgtcgggacctccggggtccgtagcggatggagtccgcagctcccggccacgttgcagaccttgggccgtggtgacgccatcgccaggcgcatcatggacggcgtccgcatcgggatggccaggatgctgctggagtccgcaaagctcggcatcctgggcggcaccagccgcgtctacgtcgcctacaccaacctcaccgccgcctggaatggctacaaagagagcgggctccccggcgctctcgacgccgtcaatcccgccagccagatggtccaagccggcgtggaggcctacgaggctgccgccgcagaggactgggaggccgccggcgccagcttgttcaaggccgggtcgatcgggatgtcgatcctggcgacggctgttggcgtcgggggagcgatcactgcgacagtgggctcgacggcaggagcggcggggagggcagccgcaagagccccctcactccctgcatatgctggcggaaaaacgtcgggagtactacggaccaccgcaggcgatacagcactgctgagcggctacaaggggccgtccgcatcgatgcctcgaggaacgccaggcatgaacggacgcatcaagtcgcatgtagaagctcatgcggctgccgtgatgcgagagcaagggatgaaggaaggaaccctgtacatcaatcgagtcccctgctctggcgccaccggatgcgacgcgatgctcccaagaatgctcccaccagatgcacaccttcgcgtggtcggtccgaatggttacgatcaagtttttgtcgggctgcccgactga

SEQ ID NO: 377 - protein, 7 aa, Synthetic construct
PKKKRKV

SEQ ID NO: 378 - protein, 8 aa, Synthetic construct
VSRKRPRP

SEQ ID NO: 379 - protein, 9 aa, Synthetic construct
PAAKRVKLD

SEQ ID NO: 380 - protein, 9 aa, Synthetic construct
KLKIKRPVK

SEQ ID NO: 381 - protein, 10 aa, Synthetic construct
EGAPPAKRAR

SEQ ID NO: 382 - protein, 12 aa, Synthetic construct
PPQPKKKPLDGE

SEQ ID NO: 383 - protein, 19 aa, Synthetic construct
MKRTADGSEFESPKKKRKV

SEQ ID NO: 384 - protein, 20 aa, Synthetic construct
AVKRPAATKKAGQAKKKKLD

SEQ ID NO: 385 - protein, 21 aa, Synthetic construct
SGGSKRTADGSEFEPKKKRKV

SEQ ID NO: 386 - protein, 25 aa, Synthetic construct
MSRRRKANPTKLSENAKKLAKEVEN

SEQ ID NO: 387 - protein, 30 aa, Synthetic construct
MDSLLMNRRKFLYQFKNVRWAKGRRETYLC

SEQ ID NO: 388 - protein, 413 aa, Synthetic construct
CLAEGTRIFDPVTGTTHRIEDVVDGRKPIHVVAAAKDGTLLARPVVSWFDQGTRDVIGLRIAGGAIVWATPDHKVLTEYGWRAAGELRKGDRVAGPGGSGNSLALSLTADQMVSALLDAEPPILYSEYDPTSPFSEASMMGLLTNLADRELVHMINWAKRVPGFVDLTLHDQAHLLECAWLEILMIGLVWRSMEHPGKLLFAPNLLLDRNQGKCVEGMVEIFDMLLATSSRFRMMNLQGEEFVCLKSIILLNSGVYTFLSSTLKSLEEKDHIHRALDKITDTLIHLMAKAGLTLQQQHQRLAQLLLILSHIRHMSNKGMEHLYSMKYKNVVPLYDLLLEMLDAHRLHAGGSGASRVQAFADALDDKFLHDMLAEELRYSVIREVLPTRRARTFDLEVEELHTLVAEGVVVHNC

SEQ ID NO: 389 - protein, 413 aa, Synthetic construct
CLAEGTRIFDPVTGTTHRIEDVVDGRKPIHVVAVAKDGTLLARPVVSWFDQGTRDVIGLRIAGGAIVWATPDHKVLTEYGWRAAGELRKGDRVAGPGGSGNSLALSLTADQMVSALLDAEPPILYSEYDPTSPFSEASMMGLLTNLADRELVHMINWAKRVPGFVDLTLHDQAHLLECAWLEILMIGLVWRSMEHPGKLLFAPNLLLDRNQGKCVEGMVEIFDMLLATSSRFRMMNLQGEEFVCLKSIILLNSGVYTFLSSTLKSLEEKDHIHRALDKITDTLIHLMAKAGLTLQQQHQRLAQLLLILSHIRHMSNKGMEHLYSMKYTNVVPLYDLLLEMLDAHRLHAGGSGASRVQAFADALDDKFLHDMLAEELRYSVIREVLPTRRARTFDLEVEELHTLVAEGVVVHNC

SEQ ID NO: 390 - protein, 413 aa, Synthetic construct
CLAEGTRIFDPVTGTTHRIEDVVDGRKPIHVVAAAKDGTLLARPVVSWFDQGTRDVIGLRIAGGATVWATPDHKVLTEYGWRAAGELRKGDRVAGPGGSGNSLALSLTADQMVSALLDAEPPIPYSEYDPTSPFSEASMMGLLTNLADRELVHMINWAKRVPGFVDLTLHDQAHLLECAWLEILMIGLVWRSMEHPGKLLFAPNLLLDRNQGKCVEGMVEIFDMLLATSSRFRMMNLQGEEFVCLKSIILLNSGVYTFLSSTLKSLEEKDHIHRALDKITDTLIHLMAKAGLTLQQQHQRLAQLLLILSHIRHMSNKGMEHLYSMKYKNVVPLYDLLLEMLDAHRLHAGGSGASRVQAFADALDDKFLHDMLAEGLRYSVIREVLPTRRARTFDLEVEELHTLVAEGVVVHNC

SEQ ID NO: 391 - protein, 413 aa, Synthetic construct
CLAEGTRIFDPVTGTTHRIEDVVDGRKPIHVVAAAKDGTLLARPVVSWFDQGTRDVIGLRIAGGATVWATPDHKVLTEYGWRAAGELRKGDRVAGPGGSGNSLALSLTADQMVSALLDAEPPILYSEYDPTSPFSEASMMGLLTNLADRELVHMINWAKRVPGFVDLTLHDQAHLLECAWLEILMIGLVWRSMEHPGKLLFAPNLLLDRNQGKCVEGMVEIFDMLLATSSRFRMMNLQGEEFVCLKSIILLNSGVYTFLSSTLKSLEEKDHIHRALDKITDTLIHLMAKAGLTLQQQHQRLAQLLLILSHIRHMSNKGMEHLYSMKYKNVVPLYDLLLEMLDAHRLHAGGSGASRVQAFADALDDKFLHDMLAEELRYSVIREVLPTRRARTFDLEVEELHTLVAEGVVVHNC

SEQ ID NO: 392 - protein, 413 aa, Synthetic construct
CLAEGTRIFDPVTGTTHRIEDVVDGRKPIHVVAAAKDGTLLARPVVSWFDQGTRDVIGLRIAGGATVWATPDHKVLTEYGWRAAGELRKGDRVAGPGGSGNSLALSLTADQMVSALLDAEPPIPYSEYDPTSPFSEASMMGLLTNLADRELVHMINWAKRVPGFVDLTLHDQAHLLECAWLEILMIGLVWRSMEHPGKLLFAPNLLLDRNQGKCVEGMVEIFDMLLATSSRFRMMNLQGEEFVCLKSIILLNSGVYTFLSSTLKSLEEKDHIHRALDKITDTLIHLMAKAGLTLQQQHQRLAQLLLILSHIRHMSNKGMEHLYSMKYKNVVPLYDLLLEMLDAHRLHAGGSGASRVQAFADALDDKFLHDMLAEELRYSVIREVLPTRRARTFDLEVEELHTLVAEGVVVHNC

SEQ ID NO: 393 - protein, 413 aa, Synthetic construct
CLAEGTRIFDPVTGTTHRIEDVVDGRKPIHVVAAAKDGTLLARPVVSWFDQGTRDVIGLRIAGGATVWATPDHKVLTEYGWRAAGELRKGDRVAGPGGSGNSLALSLTADQMVSALLDAEPPILYSEYNPTSPFSEASMMGLLTNLADRELVHMINWAKRVPGFVDLTLHDQAHLLERAWLEILMIGLVWRSMEHPGKLLFAPNLLLDRNQGKCVEGMVEIFDMLLATSSRFRMMNLQGEEFVCLKSIILLNSGVYTFLSSTLKSLEEKDHIHRALDKITDTLIHLMAKAGLTLQQQHQRLAQLLLILSHIRHMSNKGMEHLYSMKYKNVVPLYDLLLEMLDAHRLHAGGSGASRVQAFADALDDKFLHDMLAEGLRYSVIREVLPTRRARTFDLEVEELHTLVAEGVVVHNC

SEQ ID NO: 394 - protein, 413 aa, Synthetic construct
CLAEGTRIFDPVTGTTHRIEDVVDGRKPIHVVAAAKDGTLLARPVVSWFDQGTRDVIGLRIAGGAIVWATPDHKVLTEYGWRAAGELRKGDRVAGPGGSGNSLALSLTADQMVSALLDAEPPILYSEYDPTSPFSEASMMGLLTNLADRELVHMINWAKRVPGFVDLTLHDQAHLLERAWLEILMIGLVWRSMEHPGKLLFAPNLLLDRNQGKCVEGMVEIFDMLLATSSRFRMMNLQGEEFVCLKSIILLNSGVYTFLSSTLKSLEEKDHIHRALDKITDTLIHLMAKAGLTLQQQHQRLAQLLLILSHIRHMSNKGMEHLYSMKYKNVVPLYDLLLEMLDAHRLHAGGSGASRVQAFADALDDKFLHDMLAEGLRYSVIREVLPTRRARTFDLEVEELHTLVAEGVVVHNC

SEQ ID NO: 395 - protein, 413 aa, Synthetic construct
CLAEGTRIFDPVTGTTHRIEDVVDGRKPIHVVAVAKDGTLLARPVVSWFDQGTRDVIGLRIAGGATVWATPDHKVLTEYGWRAAGELRKGDRVAGPGGSGNSLALSLTADQMVSALLDAEPPILYSEYDPTSPFSEASMMGLLTNLADRELVHMINWAKRVPGFVDLTLHDQAHLLERAWLEILMIGLVWRSMEHPGKLLFAPNLLLDRNQGKCVEGMVEIFDMLLATSSRFRMMNLQGEEFVCLKSIILLNSGVYTFLSSTLKSLEEKDHIHRALDKITDTLIHLMAKAGLTLQQQHQRLAQLLLILSHIRHMSNKGMEHLYSMKYKNVVPLYDLLLEMLDAHRLHAGGSGASRVQAFADALDDKFLHDMLAEELRYSVIREVLPTRRARTFDLEVEELHTLVAEGVVVHNC

SEQ ID NO: 396 - protein, 11 aa, Synthetic construct
FQCRICMRNFS

SEQ ID NO: 397 - protein, 5 aa, Synthetic construct
HTKIH

SEQ ID NO: 398 - skipped

SEQ ID NO: 399 - protein, 8 aa, Synthetic construct
DYKDDDDK

SEQ ID NO: 400 - skipped

SEQ ID NO: 401 - protein, 11 aa, Synthetic construct
LQKKLEELELD

SEQ ID NO: 402 - protein, 49 aa, Synthetic construct
MLGFVGRVAAAPASGALRRLTPSASLPPAQLLLRAAPTAVHPVRDYAAQ

SEQ ID NO: 403 - protein, 18 aa, Synthetic construct
VDEMTKKFGTLTIHDTEK

SEQ ID NO: 404 - protein, 143 aa, Synthetic construct
MAERPFQCRICMRNFSTSGSLSRHIRTHTGEKPFACDICGRKFAQSGSLTRHTKIHTGGQRPFQCRICMRNFSRSDALSQHIRTHTGEKPFACDICGRKFARNDNRITHTKIHTGEKPFQCRICMRKFARSDHLTQHTKIHLR

SEQ ID NO: 405 - protein, 143 aa, Synthetic construct
MAERPFQCRICMRNFSQASNLISHIRTHTGEKPFACDICGRKFATSHSLTEHTKIHTGSQKPFQCRICMRNFSERSHLREHIRTHTGEKPFACDICGRKFAQSGNLTEHTKIHTGEKPFQCRICMRKFASKKALTEHTKIHLR

SEQ ID NO: 406 - protein, 143 aa, Synthetic construct
MAERPFQCRICMRNFSQASNLISHIRTHTGEKPFACDICGRKFAQRANLRAHTKIHTGSQKPFQCRICMRNFSQASNLISHIRTHTGEKPFACDICGRKFATSHSLTEHTKIHTGEKPFQCRICMRKFAERSHLREHTKIHLR

SEQ ID NO: 407 - protein, 86 aa, Synthetic construct
MAERPFQCRICMRNFSTSGSLSRHIRTHTGEKPFACDICGRKFAQSGSLTRHTKIHTGQKPFQCRICMRNFSRSDALSQHTKIHLR

SEQ ID NO: 408 - protein, 86 aa, Synthetic construct
MAERPFQCRICMRNFSQASNLISHIRTHTGEKPFACDICGRKFATSHSLTEHTKIHTGQKPFQCRICMRNFSERSHLREHTKIHLR

SEQ ID NO: 409 - protein, 85 aa, Synthetic construct
MAERPFQCRICMRNFSQRANLRAHIRTHTGEKPFACDICGRKFAQASNLISHTKIHTGQKPFQCRICMRNFSTSHSLTEHTKIHL

SEQ ID NO: 410 - protein, 143 aa, Synthetic construct
MAERPFQCRICMRNFSRSDNLSTHIRTHTGEKPFACDICGRKFADRSDLSRHTKIHTGEKPFQCRICMRKFAQSGDLTRHTKIHTGSQKPFQCRICMRNFSRSDSLSAHIRTHTGEKPFACDICGRKFAQKATRITHTKIHLR

SEQ ID NO: 411 - protein, 143 aa, Synthetic construct
MAERPFQCRICMRNFSQSSSLVRHIRTHTGEKPFACDICGRKFARSDNLVRHTKIHTGSQKPFQCRICMRNFSQAGHLASHIRTHTGEKPFACDICGRKFARKDNLKNHTKIHTGEKPFQCRICMRKFARKDALRGHTKIHLR

SEQ ID NO: 412 - protein, 143 aa, Synthetic construct
MAERPFQCRICMRNFSRSDHLTTHIRTHTGEKPFACDICGRKFAQSSSLVRHTKIHTGSQKPFQCRICMRNFSRSDNLVRHIRTHTGEKPFACDICGRKFAQAGHLASHTKIHTGEKPFQCRICMRKFARKDNLKNHTKIHLR

SEQ ID NO: 413 - protein, 32 aa, Synthetic construct
SGGSSGGSSGSETPGTSESATPESSGGSSGGS

SEQ ID NO: 414 - protein, 124 aa, Synthetic construct
  VARIANT 5..124: note = May be absent
SGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGS

SEQ ID NO: 415 - protein, 124 aa, Synthetic construct
  VARIANT 5..124: note = May be absent
GGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGS

SEQ ID NO: 416 - protein, 155 aa, Synthetic construct
  VARIANT 6..155: note = May be absent
GGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGS

SEQ ID NO: 417 - protein, 31 aa, Synthetic construct
  VARIANT 2..31: note = May be absent
GGGGGGGGGGGGGGGGGGGGGGGGGGGGGGG

SEQ ID NO: 418 - protein, 155 aa, Synthetic construct
EAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAK

SEQ ID NO: 419 - protein, 24 aa, Synthetic construct
  VARIANT 1..4: note = May be repeated between 1 and 30 times
  VARIANT 20..24: note = May be repeated between 1 and 30 times
SGGSSGSETPGTSESATPESSGGS

SEQ ID NO: 420 - protein, 93 aa, Synthetic construct
  VARIANT 4..93: note = May be absent
GGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGS

SEQ ID NO: 421 - protein, 62 aa, Synthetic construct
  VARIANT 3..62: note = May be absent
XPXPXPXPXPXPXPXPXPXPXPXPXPXPXPXPXPXPXPXPXPXPXPXPXPXPXPXPXPXPXP

SEQ ID NO: 422 - protein, 24 aa, Synthetic construct
SGGSSGSETPGTSESATPESSGGS

SEQ ID NO: 423 - protein, 104 aa, Synthetic construct
GGSGGSPGSPAGSPTSTEEGTSESATPESGPGTSTEPSEGSAPGSPAGSPTSTEEGTSTEPSEGSAPGTSTEPSEGSAPGTSESATPESGPGSEPATSGGSGGS

SEQ ID NO: 424 - protein, 24 aa, Synthetic construct
SGGSSGGSSGSETPGTSESATPES

SEQ ID NO: 425 - protein, 40 aa, Synthetic construct
SGGSSGGSSGSETPGTSESATPESSGGSSGGSSGGSSGGS

SEQ ID NO: 426 - protein, 64 aa, Synthetic construct
SGGSSGGSSGSETPGTSESATPESSGGSSGGSSGGSSGGSSGSETPGTSESATPESSGGSSGGS

SEQ ID NO: 427 - protein, 92 aa, Synthetic construct
PGSPAGSPTSTEEGTSESATPESGPGTSTEPSEGSAPGSPAGSPTSTEEGTSTEPSEGSAPGTSTEPSEGSAPGTSESATPESGPGSEPATS

SEQ ID NO: 428 - protein, 106 aa, Synthetic construct
CAAACTACCACCTACCTCCCTCACCAAAGCCCATAAAAATAAAAAATTATAACGTTTGATGGTGGATGGAGGGAGTGGTTTCGGGTATTTTTATTTTTTAATATTG

SEQ ID NO: 429 - protein, 6 aa, Synthetic construct
TGSEKP

SEQ ID NO: 430 - protein, 13 aa, Synthetic construct
YACPVESCGRSFS

SEQ ID NO: 431 - protein, 28 aa, Synthetic construct
FACDICGRKFAQSGSLTRHTKIHTGQKP

SEQ ID NO: 432 - protein, 25 aa, Synthetic construct
FQCRICMRNFSRSDNLTRHTKIHLR

SEQ ID NO: 433 - protein, 27 aa, Synthetic construct
FQCRICMRNFSTSGLSRHIRTHTGEKP

SEQ ID NO: 434 - protein, 5 aa, Synthetic construct
MAERP

SEQ ID NO: 435 - protein, 7 aa, Synthetic construct
HTKIHLR

SEQ ID NO: 436 - protein, 25 aa, Synthetic construct
FACDICGRKFADSGNLRVHTKIHLR

SEQ ID NO: 437 - RNA, 73 nt, Synthetic construct
  source 1..73: mol_type = other RNA
accagtgacatttctccacaaccaagaaaattagaaattgaattttccaattacgattcaatcgaaatgtcac

SEQ ID NO: 438 - protein, 28 aa, Synthetic construct
FQCRICMRNFSRSDNLTRHIRTHTGEKP

SEQ ID NO: 439 - protein, 6 aa, Synthetic construct
TGSQKP

SEQ ID NO: 440 - protein, 28 aa, Synthetic construct
FACDICGRKFADSGNLRVHTKIHTGEKP

SEQ ID NO: 441 - protein, 25 aa, Synthetic construct
FQCRICMRKFAQSGHLTEHTKIHLR

SEQ ID NO: 442 - protein, 11 aa, Synthetic construct
FQCRICMRKFA

SEQ ID NO: 443 - protein, 29 aa, Synthetic construct
FACDICGRKFADSGNLRVHTKIHTGSQKP

SEQ ID NO: 444 - protein, 28 aa, Synthetic construct
FQCRICMRKFAQSGHLTEHIRTHTGEKP

SEQ ID NO: 445 - protein, 25 aa, Synthetic construct
FQCRICMRNFSRRDELNVHTKIHLR

SEQ ID NO: 446 - protein, 30 aa, Synthetic construct
AIPVKRGATGETKVFTGKSNSPKSATKGGC

SEQ ID NO: 447 - protein, 30 aa, Synthetic construct
AIPVKRGATGETKVFTGKSNSPKSKTKGGC

SEQ ID NO: 448 - RNA, 68 nt, Synthetic construct
  source 1..68: mol_type = other RNA
accagtgatacctctaaaattccaaagattgaaattgaattttccaattgcgagaatcgaagtatcac

SEQ ID NO: 449 - protein, 5 aa, Synthetic construct
TGQKP

SEQ ID NO: 450 - protein, 1368 aa, Synthetic construct [sequence omitted]

SEQ ID NO: 451 - protein, 1368 aa, Synthetic construct
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 452 - DNA, 15 bp, Synthetic construct
  source 1..15: mol_type = other DNA
aatttcatatgaagt

SEQ ID NO: 453 - DNA, 15 bp, Synthetic construct
  source 1..15: mol_type = other DNA
ttaaagtatacttca

SEQ ID NO: 454 - DNA, 13 bp, Synthetic construct
  source 1..13: mol_type = other DNA
tagaagtcagggc

SEQ ID NO: 455 - DNA, 13 bp, Synthetic construct
  source 1..13: mol_type = other DNA
atcttcagtcccg

SEQ ID NO: 456 - DNA, 12 bp, Synthetic construct
  source 1..12: mol_type = other DNA
caacctccctca

SEQ ID NO: 457 - DNA, 12 bp, Synthetic construct
  source 1..12: mol_type = other DNA
gttggagggagt

SEQ ID NO: 458 - DNA, 10 bp, Synthetic construct
  source 1..10: mol_type = other DNA
ccctcaccaa

SEQ ID NO: 459 - DNA, 10 bp, Synthetic construct
  source 1..10: mol_type = other DNA
gggagtggtt

SEQ ID NO: 460 - DNA, 86 bp, Synthetic construct
  source 1..86: mol_type = other DNA
aaaacccgccacatctaccatcaccctctacatcaccgccccgttttgggcggtgtagatggtagtgggagatgtagtggcggggc

SEQ ID NO: 461 - DNA, 106 bp, Synthetic construct
  source 1..106: mol_type = other DNA
cctctctcctactcctgctcgcatctgctatagtggaggccggagcaggaacaggagagaggatgaggacgagcgtagacgatatcacctccggcctcgtccttgt

SEQ ID NO: 462 - DNA, 108 bp, Synthetic construct
  source 1..108: mol_type = other DNA
acagaccgcaacctcaacaccaccttcttcgaccccgccggaggaggagacccctgtctggcgttggagttgtggtggaagaagctggggcggcctcctcctctgggg

SEQ ID NO: 463 - DNA, 84 bp, Synthetic construct
  source 1..84: mol_type = other DNA
gagtacaccgactacggcggactaatcttcaactcctacatactcatgtggctgatgccgcctgattagaagttgaggatgtat

SEQ ID NO: 464 - DNA, 98 bp, Synthetic construct
  source 1..98: mol_type = other DNA
gtttaccacaaccaccaccccatcatactctttcacccacagcaccaatcaaatggtgttggtggtggggtagtatgagaaagtgggtgtcgtggtta

SEQ ID NO: 465 - DNA, 78 bp, Synthetic construct
  source 1..78: mol_type = other DNA
ttttaagttaaagattaagagaaccaacacctctttacaaaaattcaatttctaattctcttggttgtggagaaatgt

SEQ ID NO: 466 - DNA, 92 bp, Synthetic construct
  source 1..92: mol_type = other DNA
aaccttttaagttaaagttagagaccttaaaatctccatagtgatattggaaaattcaatttcaatctctggaattttagaggtatcactat

SEQ ID NO: 467 - DNA, 96 bp, Synthetic construct
  source 1..96: mol_type = other DNA
ccgtagcccaaacaatttcatatgaagtaaccatagctattatcctttggcatcgggtttgttaaagtatacttcattggtatcgataataggaaa

SEQ ID NO: 468 - protein, 5 aa, Synthetic construct
SYEVT

SEQ ID NO: 469 - DNA, 15 bp, Synthetic construct
  source 1..15: mol_type = other DNA
tcatatgaagtaacc

SEQ ID NO: 470 - protein, 5 aa, Synthetic construct
SYKVT

SEQ ID NO: 471 - DNA, 15 bp, Synthetic construct
  source 1..15: mol_type = other DNA
tcatataaagtaacc

SEQ ID NO: 472 - DNA, 80 bp, Synthetic construct
  source 1..80: mol_type = other DNA
ctctcctactcctgctcgcatctgctatagtggaggccgggagaggatgaggacgagcgtagacgatatcacctccggcc

SEQ ID NO: 473 - DNA, 78 bp, Synthetic construct
  source 1..78: mol_type = other DNA
gcctcctactcaggacacccacccttgggtggaggccaccggaggatgagtcctgtgggtcccaacccacctccggtg

SEQ ID NO: 474 - DNA, 84 bp, Synthetic construct
  source 1..84: mol_type = other DNA
cactcctactctccccatcagggaggaatgagtggaggcctagtgaggatgagaggggtagtccctccttactcacctccggat

SEQ ID NO: 475 - DNA, 74 bp, Synthetic construct
  source 1..74: mol_type = other DNA
accaagacctcaacccctgacccccatgcctcaggattggttctggagttggggactgggggtacggagtccta

SEQ ID NO: 476 - DNA, 70 bp, Synthetic construct
  source 1..70: mol_type = other DNA
cctggagcctccgtagacctaaccatcttctccttggacctcggaggcatctgcattggtagaagaggaa

SEQ ID NO: 477 - DNA, 72 bp, Synthetic construct
  source 1..72: mol_type = other DNA
cttggagcctcactgacttctcactatcttctcctggaacctcggagtgactgaagagtgatagaagaggac

SEQ ID NO: 478 - DNA, 93 bp, Synthetic construct
  source 1..93: mol_type = other DNA
ggcaggagccagggctgggcatagaagtcagggcagagcatctattccgtcctcggtcccgacccgtatcttcagtcccgtctcggtagataa

SEQ ID NO: 479 - DNA, 16 bp, Synthetic construct
  source 1..16: mol_type = other DNA
ctacctccctcaccaa

SEQ ID NO: 480 - DNA, 16 bp, Synthetic construct
  source 1..16: mol_type = other DNA
gatggagggagtggtt

SEQ ID NO: 481 - DNA, 19 bp, Synthetic construct
  source 1..19: mol_type = other DNA
ctacctccctcaccaaagc

SEQ ID NO: 482 - DNA, 19 bp, Synthetic construct
  source 1..19: mol_type = other DNA
gatggagggagtggtttcg

SEQ ID NO: 483 - DNA, 13 bp, Synthetic construct
  source 1..13: mol_type = other DNA
cgtagacctaacc

SEQ ID NO: 484 - DNA, 13 bp, Synthetic construct
  source 1..13: mol_type = other DNA
gcatctggattgg

SEQ ID NO: 485 - DNA, 20 bp, Synthetic construct
  source 1..20: mol_type = other DNA
tcacttcaacctccctcacc

SEQ ID NO: 486 - DNA, 20 bp, Synthetic construct
  source 1..20: mol_type = other DNA
agtgaagttggagggagtgg

SEQ ID NO: 487 - DNA, 13 bp, Synthetic construct
  source 1..13: mol_type = other DNA
atctaccatcacc

SEQ ID NO: 488 - DNA, 13 bp, Synthetic construct
  source 1..13: mol_type = other DNA
tagatggtagtgg

SEQ ID NO: 489 - DNA, 18 bp, Synthetic construct
  source 1..18: mol_type = other DNA
ctgctcgcatctgctata

SEQ ID NO: 490 - DNA, 18 bp, Synthetic construct
  source 1..18: mol_type = other DNA
gacgagcgtagacgatat

SEQ ID NO: 491 - DNA, 13 bp, Synthetic construct
  source 1..13: mol_type = other DNA
cccatcatactct

SEQ ID NO: 492 - DNA, 13 bp, Synthetic construct
  source 1..13: mol_type = other DNA
gggtagtatgaga

SEQ ID NO: 493 - DNA, 13 bp, Synthetic construct
  source 1..13: mol_type = other DNA
tactccacctcaa

SEQ ID NO: 494 - DNA, 13 bp, Synthetic construct
  source 1..13: mol_type = other DNA
atgaggtggagtt

SEQ ID NO: 495 - DNA, 12 bp, Synthetic construct
  source 1..12: mol_type = other DNA
ctccctctctcc

SEQ ID NO: 496 - DNA, 12 bp, Synthetic construct
  source 1..12: mol_type = other DNA
gagggagagagg

SEQ ID NO: 497 - DNA, 12 bp, Synthetic construct
  source 1..12: mol_type = other DNA
gtcctcatcgcc

SEQ ID NO: 498 - DNA, 12 bp, Synthetic construct
  source 1..12: mol_type = other DNA
caggagtagcgg

SEQ ID NO: 499 - DNA, 17 bp, Synthetic construct
  source 1..17: mol_type = other DNA
aggacacccagggttgg

SEQ ID NO: 500 - DNA, 17 bp, Synthetic construct
  source 1..17: mol_type = other DNA
tcctgtgggtcccaacc

SEQ ID NO: 501 - DNA, 20 bp, Synthetic construct
  source 1..20: mol_type = other DNA
tccccatcagggaggaatga

SEQ ID NO: 502 - DNA, 20 bp, Synthetic construct
  source 1..20: mol_type = other DNA
aggggtagtccctccttact

SEQ ID NO: 503 - DNA, 12 bp, Synthetic construct
  source 1..12: mol_type = other DNA
aacccctgaccc

SEQ ID NO: 504 - DNA, 12 bp, Synthetic construct
  source 1..12: mol_type = other DNA
ttggggactggg

SEQ ID NO: 505 - DNA, 14 bp, Synthetic construct
  source 1..14: mol_type = other DNA
actgacttctcact

SEQ ID NO: 506 - DNA, 14 bp, Synthetic construct
  source 1..14: mol_type = other DNA
tgactgaagagtga

SEQ ID NO: 507 - DNA, 11 bp, Synthetic construct
  source 1..11: mol_type = other DNA
tagaagtcagg

SEQ ID NO: 508 - DNA, 11 bp, Synthetic construct
  source 1..11: mol_type = other DNA
atcttcagtcc

SEQ ID NO: 509 - RNA, 78 nt, Synthetic construct
  source 1..78: mol_type = other RNA
accacaattcttctccttaacttggagactgacatttcaaaattcaaaatacgctaatggcccgggacggtagaattg

SEQ ID NO: 510 - DNA, 94 bp, Synthetic construct
  source 1..94: mol_type = other DNA
taagttttatgcgattaccgggccctgccatcttaacaaaccctgttattcaaaatacgctaatggcccgggacggtagaattgtttgggacaa

SEQ ID NO: 511 - protein, 521 aa, Synthetic construct
MLGFVGRVAAAPASGALRRLTPSASLPPAQLLLRAAPTAVHPVRDYAAQDYKDDDDKVDEMTKKFGTLTIHDTEKGSLQKKLEELELDAAMAERPFQCRICMRNFSTSGSLSRHIRTHTGEKPFACDICGRKFAQSGSLTRHTKIHTGQKPFQCRICMRNFSRSDALSQHTKIHLRGSGGGGSGGSGGSGSYALGPYQISAPQLPAYNGQTVGTFYYVNDAGGLESKVFSSGGPTPYPNYANAGHVEGQSALFMRDNGISEGLVFHNNPEGTCGFCVNMTETLLPENAKMTVVPPEGSGGSTNLSDIIEKETGKQLVIQESILMLPEEVEEVIGNKPESDILVHTAYDESTDENVMLLTSDAPEYKPWALVIQDSNGENKIKMLGSGATNFSLLKQAGDVEENPGPMASVLTPLLLRGLTGSARRLPVPRAKIHSLGSTNLSDIIEKETGKQLVIQESILMLPEEVEEVIGNKPESDILVHTAYDESTDENVMLLTSDAPEYKPWALVIQDSNGENKIKML

SEQ ID NO: 512 - protein, 521 aa, Synthetic construct
MLGFVGRVAAAPASGALRRLTPSASLPPAQLLLRAAPTAVHPVRDYAAQDYKDDDDKVDEMTKKFGTLTIHDTEKGSLQKKLEELELDAAMAERPYKCEECGKAFNTSGSLSRHMKIHTGEKPYKCEECGKAFNQSGSLTRHMKIHTGEKPYKCEECGKAFNRSDALSQHMKIHLRGSGGGGSGGSGGSGSYALGPYQISAPQLPAYNGQTVGTFYYVNDAGGLESKVFSSGGPTPYPNYANAGHVEGQSALFMRDNGISEGLVFHNNPEGTCGFCVNMTETLLPENAKMTVVPPEGSGGSTNLSDIIEKETGKQLVIQESILMLPEEVEEVIGNKPESDILVHTAYDESTDENVMLLTSDAPEYKPWALVIQDSNGENKIKMLGSGATNFSLLKQAGDVEENPGPMASVLTPLLLRGLTGSARRLPVPRAKIHSLGSTNLSDIIEKETGKQLVIQESILMLPEEVEEVIGNKPESDILVHTAYDESTDENVMLLTSDAPEYKPWALVIQDSNGENKIKML

SEQ ID NO: 513 - protein, 521 aa, Synthetic construct
MLGFVGRVAAAPASGALRRLTPSASLPPAQLLLRAAPTAVHPVRDYAAQDYKDDDDKVDEMTKKFGTLTIHDTEKGSLQKKLEELELDAAMAERPYKCEECGKAFNTSGSLSRHMKIHTGEKPYKCEECGKAFNQSGSLTRHMKIHTGEKPYKCEECGKAFNRSDALSQHMKIHLRGSGGGGSGGSGGSGSYALGPYQISAPQLPAYNGQTVGTFYYVNDAGGLESKVFSSGGPTPYPNYANAGHVEGQSALFMRDNGISEGLVFHNNPEGTCGFCVNMIETLLPENAKMTVVPPKGSGGSTNLSDIIEKETGKQLVIQESILMLPEEVEEVIGNKPESDILVHTAYDESTDENVMLLTSDAPEYKPWALVIQDSNGENKIKMLGSGATNFSLLKQAGDVEENPGPMASVLTPLLLRGLTGSARRLPVPRAKIHSLGSTNLSDIIEKETGKQLVIQESILMLPEEVEEVIGNKPESDILVHTAYDESTDENVMLLTSDAPEYKPWALVIQDSNGENKIKML

SEQ ID NO: 514 - protein, 384 aa, Synthetic construct
MLGFVGRVAAAPASGALRRLTPSASLPPAQLLLRAAPTAVHPVRDYAAQDYKDDDDKVDEMTKKFGTLTIHDTEKGSLQKKLEELELDAAMAERPYKCEECGKAFNTSGSLSRHMKIHTGEKPYKCEECGKAFNQSGSLTRHMKIHTGEKPYKCEECGKAFNRSDALSQHMKIHLRGSGGGGSGGSGGSGSYALGPYQISAPQLPAYNGQTVGTFYYVNDAGGLESKVFSSGGPTPYPNYANAGHVEGQSALFMRDNGISEGLVFHNNPEGTCGFCVNMIETLLPENAKMTVVPPKGSGGSTNLSDIIEKETGKQLVIQESILMLPEEVEEVIGNKPESDILVHTAYDESTDENVMLLTSDAPEYKPWALVIQDSNGENKIKML

SEQ ID NO: 515 - protein, 276 aa, Synthetic construct
MLGFVGRVAAAPASGALRRLTPSASLPPAQLLLRAAPTAVHPVRDYAAQTSVDEMTKKFGTLTIHDTEKAAMAERPFQCRICMRNFSQASNLISHIRTHTGEKPFACDICGRKFATSHSLTEHTKIHTGQKPFQCRICMRNFSERSHLREHTKIHLRGSAIPVKRGATGETKVFTGNSNSPKSPTKGGCSGGSTNLSDIIEKETGKQLVIQESILMLPEEVEEVIGNKPESDILVHTAYDESTDENVMLLTSDAPEYKPWALVIQDSNGENKIKML

SEQ ID NO: 516 - protein, 287 aa, Synthetic construct
MLGFVGRVAAAPASGALRRLTPSASLPPAQLLLRAAPTAVHPVRDYAAQTSVDEMTKKFGTLTIHDTEKAAMAERPFQCRICMRNFSQASNLISHIRTHTGEKPFACDICGRKFATSHSLTEHTKIHTGQKPFQCRICMRNFSERSHLREHTKIHLRGSGGGGSGGSGGSAIPVKRGATGETKVFTGNSNSPKSPTKGGCSGGSTNLSDIIEKETGKQLVIQESILMLPEEVEEVIGNKPESDILVHTAYDESTDENVMLLTSDAPEYKPWALVIQDSNGENKIKML

SEQ ID NO: 517 - protein, 293 aa, Synthetic construct
MLGFVGRVAAAPASGALRRLTPSASLPPAQLLLRAAPTAVHPVRDYAAQDYKDDDDKVDEMTKKFGTLTIHDTEKAAMAERPFQCRICMRNFSQASNLISHIRTHTGEKPFACDICGRKFATSHSLTEHTKIHTGQKPFQCRICMRNFSERSHLREHTKIHLRGSGGGGSGGSGGSAIPVKRGATGETKVFTGNSNSPKSPTKGGCSGGSTNLSDIIEKETGKQLVIQESILMLPEEVEEVIGNKPESDILVHTAYDESTDENVMLLTSDAPEYKPWALVIQDSNGENKIKML

SEQ ID NO: 518 - protein, 306 aa, Synthetic construct
MLGFVGRVAAAPASGALRRLTPSASLPPAQLLLRAAPTAVHPVRDYAAQDYKDDDDKVDEMTKKFGTLTIHDTEKGSLQKKLEELELDAAMAERPFQCRICMRNFSQASNLISHIRTHTGEKPFACDICGRKFATSHSLTEHTKIHTGQKPFQCRICMRNFSERSHLREHTKIHLRGSGGGGSGGSGGSAIPVKRGATGETKVFTGNSNSPKSPTKGGCSGGSTNLSDIIEKETGKQLVIQESILMLPEEVEEVIGNKPESDILVHTAYDESTDENVMLLTSDAPEYKPWALVIQDSNGENKIKML

SEQ ID NO: 519 - protein, 443 aa, Synthetic construct
MLGFVGRVAAAPASGALRRLTPSASLPPAQLLLRAAPTAVHPVRDYAAQDYKDDDDKVDEMTKKFGTLTIHDTEKGSLQKKLEELELDAAMAERPFQCRICMRNFSQASNLISHIRTHTGEKPFACDICGRKFATSHSLTEHTKIHTGQKPFQCRICMRNFSERSHLREHTKIHLRGSGGGGSGGSGGSAIPVKRGATGETKVFTGNSNSPKSPTKGGCSGGSTNLSDIIEKETGKQLVIQESILMLPEEVEEVIGNKPESDILVHTAYDESTDENVMLLTSDAPEYKPWALVIQDSNGENKIKMLGSGATNFSLLKQAGDVEENPGPMASVLTPLLLRGLTGSARRLPVPRAKIHSLGSTNLSDIIEKETGKQLVIQESILMLPEEVEEVIGNKPESDILVHTAYDESTDENVMLLTSDAPEYKPWALVIQDSNGENKIKML

SEQ ID NO: 520 - protein, 443 aa, Synthetic construct
MLGFVGRVAAAPASGALRRLTPSASLPPAQLLLRAAPTAVHPVRDYAAQDYKDDDDKVDEMTKKFGTLTIHDTEKGSLQKKLEELELDAAMAERPYKCEECGKAFNQASNLISHMKIHTGEKPYKCEECGKAFNTSHSLTEHMKIHTGEKPYKCEECGKAFNERSHLREHMKIHLRGSGGGGSGGSGGSAIPVKRGATGETKVFTGNSNSPKSPTKGGCSGGSTNLSDIIEKETGKQLVIQESILMLPEEVEEVIGNKPESDILVHTAYDESTDENVMLLTSDAPEYKPWALVIQDSNGENKIKMLGSGATNFSLLKQAGDVEENPGPMASVLTPLLLRGLTGSARRLPVPRAKIHSLGSTNLSDIIEKETGKQLVIQESILMLPEEVEEVIGNKPESDILVHTAYDESTDENVMLLTSDAPEYKPWALVIQDSNGENKIKML

SEQ ID NO: 521 - protein, 443 aa, Synthetic construct
MLGFVGRVAAAPASGALRRLTPSASLPPAQLLLRAAPTAVHPVRDYAAQDYKDDDDKVDEMTKKFGTLTIHDTEKGSLQKKLEELELDAAMAERPYKCEECGKAFNQASNLISHMKIHTGEKPYKCEECGKAFNTSHSLTEHMKIHTGEKPYKCEECGKAFNERSHLREHMKIHLRGSGGGGSGGSGGSAIPVKRGATGETKVFIGNSNSPKSPTKGGCSGGSTNLSDIIEKETGKQLVIQESILMLPEEVEEVIGNKPESDILVHTAYDESTDENVMLLTSDAPEYKPWALVIQDSNGENKIKMLGSGATNFSLLKQAGDVEENPGPMASVLTPLLLRGLTGSARRLPVPRAKIHSLGSTNLSDIIEKETGKQLVIQESILMLPEEVEEVIGNKPESDILVHTAYDESTDENVMLLTSDAPEYKPWALVIQDSNGENKIKML

SEQ ID NO: 522 - protein, 306 aa, Synthetic construct
MLGFVGRVAAAPASGALRRLTPSASLPPAQLLLRAAPTAVHPVRDYAAQDYKDDDDKVDEMTKKFGTLTIHDTEKGSLQKKLEELELDAAMAERPYKCEECGKAFNQASNLISHMKIHTGEKPYKCEECGKAFNTSHSLTEHMKIHTGEKPYKCEECGKAFNERSHLREHMKIHLRGSGGGGSGGSGGSAIPVKRGATGETKVFIGNSNSPKSPTKGGCSGGSTNLSDIIEKETGKQLVIQESILMLPEEVEEVIGNKPESDILVHTAYDESTDENVMLLTSDAPEYKPWALVIQDSNGENKIKML

SEQ ID NO: 523 - protein, 362 aa, Synthetic construct
MLGFVGRVAAAPASGALRRLTPSASLPPAQLLLRAAPTAVHPVRDYAAQVDEMTKKFGTLTIHDTEKAASGGSGSYALGPYQISAPQLPAYNGQTVGTFYYVNDAGGLESKVFSSGGPTPYPNYANAGHVEGQSALFMRDNGISEGLVFHNNPEGTCGFCVNMTETLLPENAKMTVVPPEGGSMAERPFQCRICMRNFSRSDNLVRHIRTHTGEKPFACDICGRKFAQSSSLVRHTKIHTGQKPFQCRICMRNFSTSGNLVRHTKIHLRSGGSGGSGGSTNLSDIIEKETGKQLVIQESILMLPEEVEEVIGNKPESDILVHTAYDESTDENVMLLTSDAPEYKPWALVIQDSNGENKIKML

SEQ ID NO: 524 - protein, 531 aa, Synthetic construct
MLGFVGRVAAAPASGALRRLTPSASLPPAQLLLRAAPTAVHPVRDYAAQDYKDDDDKVDEMTKKFGTLTIHDTEKGSLQKKLEELELDAASGGSGSYALGPYQISAPQLPAYNGQTVGTFYYVNDAGGLESKVFSSGGPTPYPNYANAGHVEGQSALFMRDNGISEGLVFHNNPEGTCGFCVNMTETLLPENAKMTVVPPEGGSGGGGSGGSGGSMAERPFQCRICMRNFSRSDNLVRHIRTHTGEKPFACDICGRKFAQSSSLVRHTKIHTGQKPFQCRICMRNFSTSGNLVRHTKIHLRSGGSGGSGGSTNLSDIIEKETGKQLVIQESILMLPEEVEEVIGNKPESDILVHTAYDESTDENVMLLTSDAPEYKPWALVIQDSNGENKIKMLGSGATNFSLLKQAGDVEENPGPMASVLTPLLLRGLTGSARRLPVPRAKIHSLGSTNLSDIIEKETGKQLVIQESILMLPEEVEEVIGNKPESDILVHTAYDESTDENVMLLTSDAPEYKPWALVIQDSNGENKIKML

SEQ ID NO: 525 - protein, 531 aa, Synthetic construct
MLGFVGRVAAAPASGALRRLTPSASLPPAQLLLRAAPTAVHPVRDYAAQDYKDDDDKVDEMTKKFGTLTIHDTEKGSLQKKLEELELDAASGGSGSYALGPYQISAPQLPAYNGQTVGTFYYVNDAGGLESKVFSSGGPTPYPNYANAGHVEGQSALFMRDNGISEGLVFHNNPEGTCGFCVNMTETLLPENAKMTVVPPEGGSGGGGSGGSGGSMAERPYKCEECGKAFNRSDNLVRHMKIHTGEKPYKCEECGKAFNQSSSLVRHMKIHTGEKPYKCEECGKAFNTSGNLVRHTKIHLRSGGSGGSGGSTNLSDIIEKETGKQLVIQESILMLPEEVEEVIGNKPESDILVHTAYDESTDENVMLLTSDAPEYKPWALVIQDSNGENKIKMLGSGATNFSLLKQAGDVEENPGPMASVLTPLLLRGLTGSARRLPVPRAKIHSLGSTNLSDIIEKETGKQLVIQESILMLPEEVEEVIGNKPESDILVHTAYDESTDENVMLLTSDAPEYKPWALVIQDSNGENKIKML

SEQ ID NO: 526 - protein, 531 aa, Synthetic construct
MLGFVGRVAAAPASGALRRLTPSASLPPAQLLLRAAPTAVHPVRDYAAQDYKDDDDKVDEMTKKFGTLTIHDTEKGSLQKKLEELELDAASGGSGSYALGPYQISAPQLPAYNGQTVGTFYYVNDAGGLESKVFSSGGPTPYPNYANAGHVEGQSALFMRDNGISEGLVFHNNPEGTCGFCVNMIETLLPENAKMTVVPPKGGSGGGGSGGSGGSMAERPYKCEECGKAFNRSDNLVRHMKIHTGEKPYKCEECGKAFNQSSSLVRHMKIHTGEKPYKCEECGKAFNTSGNLVRHTKIHLRSGGSGGSGGSTNLSDIIEKETGKQLVIQESILMLPEEVEEVIGNKPESDILVHTAYDESTDENVMLLTSDAPEYKPWALVIQDSNGENKIKMLGSGATNFSLLKQAGDVEENPGPMASVLTPLLLRGLTGSARRLPVPRAKIHSLGSTNLSDIIEKETGKQLVIQESILMLPEEVEEVIGNKPESDILVHTAYDESTDENVMLLTSDAPEYKPWALVIQDSNGENKIKML

SEQ ID NO: 527 - protein, 394 aa, Synthetic construct
MLGFVGRVAAAPASGALRRLTPSASLPPAQLLLRAAPTAVHPVRDYAAQDYKDDDDKVDEMTKKFGTLTIHDTEKGSLQKKLEELELDAASGGSGSYALGPYQISAPQLPAYNGQTVGTFYYVNDAGGLESKVFSSGGPTPYPNYANAGHVEGQSALFMRDNGISEGLVFHNNPEGTCGFCVNMIETLLPENAKMTVVPPKGGSGGGGSGGSGGSMAERPYKCEECGKAFNRSDNLVRHMKIHTGEKPYKCEECGKAFNQSSSLVRHMKIHTGEKPYKCEECGKAFNTSGNLVRHTKIHLRSGGSGGSGGSTNLSDIIEKETGKQLVIQESILMLPEEVEEVIGNKPESDILVHTAYDESTDENVMLLTSDAPEYKPWALVIQDSNGENKIKML

SEQ ID NO: 528 - protein, 284 aa, Synthetic construct
MLGFVGRVAAAPASGALRRLTPSASLPPAQLLLRAAPTAVHPVRDYAAQVDEMTKKFGTLTIHDTEKAASGGSAIPVKRGATGETKVFTGNSNSPKSPTKGGCGSMAERPFQCRICMRNFSQSSNLVRHIRTHTGEKPFACDICGRKFATSGHLVRHTKIHTGQKPFQCRICMRNFSRSDELVRHTKIHLRSGGSGGSGGSTNLSDIIEKETGKQLVIQESILMLPEEVEEVIGNKPESDILVHTAYDESTDENVMLLTSDAPEYKPWALVIQDSNGENKIKML

SEQ ID NO: 529 - protein, 453 aa, Synthetic construct
MLGFVGRVAAAPASGALRRLTPSASLPPAQLLLRAAPTAVHPVRDYAAQDYKDDDDKVDEMTKKFGTLTIHDTEKGSLQKKLEELELDAASGGSAIPVKRGATGETKVFTGNSNSPKSPTKGGCGSGGGGSGGSGGSMAERPFQCRICMRNFSQSSNLVRHIRTHTGEKPFACDICGRKFATSGHLVRHTKIHTGQKPFQCRICMRNFSRSDELVRHTKIHLRSGGSGGSGGSTNLSDIIEKETGKQLVIQESILMLPEEVEEVIGNKPESDILVHTAYDESTDENVMLLTSDAPEYKPWALVIQDSNGENKIKMLGSGATNFSLLKQAGDVEENPGPMASVLTPLLLRGLTGSARRLPVPRAKIHSLGSTNLSDIIEKETGKQLVIQESILMLPEEVEEVIGNKPESDILVHTAYDESTDENVMLLTSDAPEYKPWALVIQDSNGENKIKML

SEQ ID NO: 530 - protein, 453 aa, Synthetic construct
MLGFVGRVAAAPASGALRRLTPSASLPPAQLLLRAAPTAVHPVRDYAAQDYKDDDDKVDEMTKKFGTLTIHDTEKGSLQKKLEELELDAASGGSAIPVKRGATGETKVFTGNSNSPKSPTKGGCGSGGGGSGGSGGSMAERPYACPECGKSFSQSSNLVRHIRTHTGEKPYACPECGKSFSTSGHLVRHIRTHTGEKPYACPECGKSFSRSDELVRHTKIHLRSGGSGGSGGSTNLSDIIEKETGKQLVIQESILMLPEEVEEVIGNKPESDILVHTAYDESTDENVMLLTSDAPEYKPWALVIQDSNGENKIKMLGSGATNFSLLKQAGDVEENPGPMASVLTPLLLRGLTGSARRLPVPRAKIHSLGSTNLSDIIEKETGKQLVIQESILMLPEEVEEVIGNKPESDILVHTAYDESTDENVMLLTSDAPEYKPWALVIQDSNGENKIKML

SEQ ID NO: 531 - protein, 453 aa, Synthetic construct
MLGFVGRVAAAPASGALRRLTPSASLPPAQLLLRAAPTAVHPVRDYAAQDYKDDDDKVDEMTKKFGTLTIHDTEKGSLQKKLEELELDAASGGSAIPVKRGATGETKVFIGNSNSPKSPTKGGCGSGGGGSGGSGGSMAERPYACPECGKSFSQSSNLVRHIRTHTGEKPYACPECGKSFSTSGHLVRHIRTHTGEKPYACPECGKSFSRSDELVRHTKIHLRSGGSGGSGGSTNLSDIIEKETGKQLVIQESILMLPEEVEEVIGNKPESDILVHTAYDESTDENVMLLTSDAPEYKPWALVIQDSNGENKIKMLGSGATNFSLLKQAGDVEENPGPMASVLTPLLLRGLTGSARRLPVPRAKIHSLGSTNLSDIIEKETGKQLVIQESILMLPEEVEEVIGNKPESDILVHTAYDESTDENVMLLTSDAPEYKPWALVIQDSNGENKIKML

SEQ ID NO: 532 - protein, 316 aa, Synthetic construct
MLGFVGRVAAAPASGALRRLTPSASLPPAQLLLRAAPTAVHPVRDYAAQDYKDDDDKVDEMTKKFGTLTIHDTEKGSLQKKLEELELDAASGGSAIPVKRGATGETKVFIGNSNSPKSPTKGGCGSGGGGSGGSGGSMAERPYACPECGKSFSQSSNLVRHIRTHTGEKPYACPECGKSFSTSGHLVRHIRTHTGEKPYACPECGKSFSRSDELVRHTKIHLRSGGSGGSGGSTNLSDIIEKETGKQLVIQESILMLPEEVEEVIGNKPESDILVHTAYDESTDENVMLLTSDAPEYKPWALVIQDSNGENKIKML

SEQ ID NO: 533 - protein, 363 aa, Synthetic construct
MKRTADGSEFESPKKKRKVSGSPAAKRVKLDSGSKRTADGSEFESPKKKRKVSGSPAAKRVKLDSGSAAMAERPFACDICGRKFARSDNLVRHIRTHTGEKPFACDICGRKFAREDNLHTHIRTHTGEKPFACDICGRKFARSDNLVRHTKIHLRGSGGGGSGGSGGSGSYALGPYQISAPQLPAYNGQTVGTFYYVNDAGGLESKVFSSGGPTPYPNYANAGHVEGQSALFMRDNGISEGLVFHNNPEGTCGFCVNMIETLLPENAKMTVVPPKGSGGSTNLSDIIEKETGKQLVIQESILMLPEEVEEVIGNKPESDILVHTAYDESTDENVMLLTSDAPEYKPWALVIQDSNGENKIKML

SEQ ID NO: 534 - protein, 369 aa, Synthetic construct
MKRTADGSEFESPKKKRKVSGSPAAKRVKLDSGSKRTADGSEFESPKKKRKVSGSPAAKRVKLDSGSAAMAERPFACDICGRKFARNDALTEHIRTHTGEKPFACDICGRKFATSGELVRHIRTHTGEKPFACDICGRKFARTDTLRDHIRTHTGEKPFACDICGRKFADCRDLARHIRTHTGEKPFACDICGRKFARSDNLVRHIRTHTGEKPFACDICGRKFARSDELVRHTKIHLRGSGGGGSGGSGGSAIPVKRGATGETKVFIGNSNSPKSPTKGGCSGGSTNLSDIIEKETGKQLVIQESILMLPEEVEEVIGNKPESDILVHTAYDESTDENVMLLTSDAPEYKPWALVIQDSNGENKIKML

SEQ ID NO: 535 - protein, 373 aa, Synthetic construct
MKRTADGSEFESPKKKRKVSGSPAAKRVKLDSGSKRTADGSEFESPKKKRKVSGSPAAKRVKLDSGSAASGGSGSYALGPYQISAPQLPAYNGQTVGTFYYVNDAGGLESKVFSSGGPTPYPNYANAGHVEGQSALFMRDNGISEGLVFHNNPEGTCGFCVNMIETLLPENAKMTVVPPKGGSGGGGSGGSGGSMAERPFACDICGRKFAQSGDLRRHIRTHTGEKPFACDICGRKFARSDHLTTHIRTHTGEKPFACDICGRKFADPGHLVRHTKIHLRSGGSGGSGGSTNLSDIIEKETGKQLVIQESILMLPEEVEEVIGNKPESDILVHTAYDESTDENVMLLTSDAPEYKPWALVIQDSNGENKIKML

SEQ ID NO: 536 - protein, 379 aa, Synthetic construct
MKRTADGSEFESPKKKRKVSGSPAAKRVKLDSGSKRTADGSEFESPKKKRKVSGSPAAKRVKLDSGSAASGGSAIPVKRGATGETKVFIGNSNSPKSPTKGGCGSGGGGSGGSGGSMAERPFACDICGRKFARLRDIQFHIRTHTGEKPFACDICGRKFADPGHLVRHIRTHTGEKPFACDICGRKFATSGNLVRHIRTHTGEKPFACDICGRKFAQKSSLIAHIRTHTGEKPFACDICGRKFAQSGDLRRHIRTHTGEKPFACDICGRKFAQASNLISHTKIHLRSGGSGGSGGSTNLSDIIEKETGKQLVIQESILMLPEEVEEVIGNKPESDILVHTAYDESTDENVMLLTSDAPEYKPWALVIQDSNGENKIKML

SEQ ID NO: 537 - DNA, 209 bp, Homo sapiens
  source 1..209: mol_type = genomic DNA
ctttacagtgaaatgccccaactaaatactaccgtatggcccaccataattacccccatactccttacactattcctcatcacccaactaaaaatattaaacacaaactaccacctacctccctcaccaaagcccataaaaataaaaaattataacaaaccctgagaaccaaaatgaacgaaaatctgttcgcttcattcattgccccc

SEQ ID NO: 538 - DNA, 210 bp, Homo sapiens
  source 1..210: mol_type = genomic DNA
cgggtccatcatccacaaccttaacaatgaacaagatattcgaaaaataggaggactactcaaaaccatacctctcacttcaacctccctcaccattggcagcctagcattagcaggaatacctttcctcacaggtttctactccaaagaccacatcatcgaaaccgcaaacatatcatacacaaacgcctgagccctatctattactct

SEQ ID NO: 539 - DNA, 217 bp, Homo sapiens
  source 1..217: mol_type = genomic DNA
aaagtttaccacaaccaccaccccatcatactctttcacccacagcaccaatcctacctccatcgctaaccccactaaaacactcaccaagacctcaacccctgacccccatgcctcaggatactcctcaatagccatcgctgtagtatatccaaagacaaccatcattccccctaaataaattaaaaaaactattaaacccatataacctccccca

SEQ ID NO: 540 - DNA, 213 bp, Homo sapiens
  source 1..213: mol_type = genomic DNA
cctactcctgctcgcatctgctatagtggaggccggagcaggaacaggttgaacagtctaccctcccttagcagggaactactcccaccctggagcctccgtagacctaaccatcttctccttacacctagcaggtgtctcctctatcttaggggccatcaatttcatcacaacaattatcaatataaaaccccctgccataacccaatacca

SEQ ID NO: 541 - DNA, 226 bp, Homo sapiens
  source 1..226: mol_type = genomic DNA
ggctatatacaactacgcaaaggccccaacgttgtaggcccctacgggctactacaacccttcgctgacgccataaaactcttcaccaaagagcccctaaaacccgccacatctaccatcaccctctacatcaccgccccgaccttagctctcaccatcgctcttctactatgaacccccctccccatacccaaccccctggtcaacctcaacctaggcctcctat

SEQ ID NO: 542 - DNA, 217 bp, Homo sapiens
  source 1..217: mol_type = genomic DNA
aatcggaggctttggcaactgactagttcccctaataatcggtgcccccgatatggcgtttccccgcataaacaacataagcttctgactcttacctccctctctcctactcctgctcgcatctgctatagtggaggccggagcaggaacaggttgaacagtctaccctcccttagcagggaactactcccaccctggagcctccgtagacctaacc

SEQ ID NO: 543 - DNA, 222 bp, Homo sapiens
  source 1..222: mol_type = genomic DNA
ataccaaacgcccctcttcgtctgatccgtcctaatcacagcagtcctacttctcctatctctcccagtcctagctgctggcatcactatactactaacagaccgcaacctcaacaccaccttcttcgaccccgccggaggaggagaccccattctataccaacacctattctgatttttcggtcaccctgaagtttatattcttatcctaccaggcttcgg

SEQ ID NO: 544 - DNA, 213 bp, Homo sapiens
  source 1..213: mol_type = genomic DNA
gcatcctttacataacagacgaggtcaacgatccctcccttaccatcaaatcaattggccaccaatggtactgaacctacgagtacaccgactacggcggactaatcttcaactcctacatacttcccccattattcctagaaccaggcgacctgcgactccttgacgttgacaatcgagtagtactcccgattgaagcccccattcgtataa

SEQ ID NO: 545 - DNA, 228 bp, Homo sapiens
  source 1..228: mol_type = genomic DNA
cccataatcatacaaagcccccgcaccaataggatcctcccgaatcaaccctgacccctctccttcataaattattcagcttcctacactattaaagtttaccacaaccaccaccccatcatactctttcacccacagcaccaatcctacctccatcgctaaccccactaaaacactcaccaagacctcaacccctgacccccatgcctcaggatactcctcaatagc

SEQ ID NO: 546 - DNA, 202 bp, Homo sapiens
  source 1..202: mol_type = genomic DNA
gacttcaaactctactcccactaatagctttttgatgacttctagcaagcctcgctaacctcgccttaccccccactattaacctactgggagaactctctgtgctagtaaccacgttctcctgatcaaatatcactctcctacttacaggactcaacatactagtcacagccctatactccctctacatatttaccacaac

SEQ ID NO: 547 - DNA, 247 bp, Homo sapiens
  source 1..247: mol_type = genomic DNA
ggagcaaaccacagtttcatgcccatcgtcctagaattaattcccctaaaaatctttgaaatagggcccgtatttaccctatagcaccccctctaccccctctagagcccactgtaaagctaacttagcattaaccttttaagttaaagattaagagaaccaacacctctttacagtgaaatgccccaactaaatactaccgtatggcccaccataattacccccatactccttacactattcctca

SEQ ID NO: 548 - DNA, 211 bp, Mus musculus
  source 1..211: mol_type = genomic DNA
gatctaaccatagctttatgcccattgtcctagaaatggttccactaaaatatttcgaaaactgatctgcttcaataatttaatttcactatgaagctaagagcgttaaccttttaagttaaagttagagaccttaaaatctccatagtgatatgccacaactagatacatcaacatgatttatcacaattatctcatcaataattaccct

SEQ ID NO: 549 - DNA, 236 bp, Mus musculus
  source 1..236: mol_type = genomic DNA
ctagcctatcagtttactccattctatgatcaggatgagcctcaaactccaaatactcactattcggagctttacgagccgtagcccaaacaatttcatatgaagtaaccatagctattatccttttatcagttctattaataaatggatcctactctctacaaacacttattacaacccaagaacacatatgattacttctgccagcctgacccatagccataatatgatttatc

SEQ ID NO: 550 - DNA, 246 bp, Homo sapiens
  source 1..246: mol_type = genomic DNA
accatcgctcttctactatgaacccccctccccatacccaaccccctggtcaacctcaacctaggcctcctatttattctagccacctctagcctagccgtttactcaatcctctgatcagggtgagcatcaaactcaaactacgccctgatcggcgcactgcgagcagtagcccaaacaatctcatatgaagtcaccctagccatcattctactatcaacattactaataagtggctcctttaac

SEQ ID NO: 551 - DNA, 231 bp, Homo sapiens
  source 1..231: mol_type = genomic DNA
gggccattatcgaagaattcacaaaaaacaatagcctcatcatccccaccatcatagccaccatcaccctccttaacctttacttctacctacgcctaatctactccacctcaatcacactactccccatatctaacaacgtaaaaataaaatgacagtttgaacatacaaaacccaccccattcctccccacactcatcgcccttaccacgctactcctacctatctccc

SEQ ID NO: 552 - DNA, 216 bp, Homo sapiens
  source 1..216: mol_type = genomic DNA
tcataaccctcaacacccactccctcttagccaatattgtgcctattgccatactagtctttgccgcctgcgaagcagcggtgggcctagccctactagtctcaatctccaacacatatggcctagactacgtacataacctaaacctactccaatgctaaaactaatcgtcccaacaattatattactaccactgacatgactttccaaaaaaca

SEQ ID NO: 553 - DNA, 222 bp, Homo sapiens
  source 1..222: mol_type = genomic DNA
cttatccagtgaaccactatcacgaaaaaaactctacctctctatactaatctccctacaaatctccttaattataacattcacagccacagaactaatcatattttatatcttcttcgaaaccacacttatccccaccttggctatcatcacccgatgaggcaaccagccagaacgcctgaacgcaggcacatacttcctattctacaccctagtaggctc

SEQ ID NO: 554 - DNA, 231 bp, Homo sapiens
  source 1..231: mol_type = genomic DNA
ccttcttgctcatcagttgatgatacgcccgagcagatgccaacacagcagccattcaagcaatcctatacaaccgtatcggcgatatcggtttcatcctcgccttagcatgatttatcctacactccaactcatgagacccacaacaaatagcccttctaaacgctaatccaagcctcaccccactactaggcctcctcctagcagcagcaggcaaatcagcccaattag

SEQ ID NO: 555 - DNA, 230 bp, Homo sapiens
  source 1..230: mol_type = genomic DNA
gctattacctaaaacaatttcacagcaccaaatctccacctccatcatcacctcaacccaaaaaggcataattaaactttacttcctctctttcttcttcccactcatcctaaccctactcctaatcacataacctattcccccgagcaatctcaattacaatatatacaccaacaaacaatgttcaaccagtaactactactaatcaacgcccataatcatacaaagcc

SEQ ID NO: 556 - DNA, 213 bp, Homo sapiens
  source 1..213: mol_type = genomic DNA
catcataatcggaggctttggcaactgactagttcccctaataatcggtgcccccgatatggcgtttccccgcataaacaacataagcttctgactcttacctccctctctcctactcctgctcgcatctgctatagtggaggccggagcaggaacaggttgaacagtctaccctcccttagcagggaactactcccaccctggagcctccgt

SEQ ID NO: 557 - DNA, 228 bp, Homo sapiens
  source 1..228: mol_type = genomic DNA
tgcccttttcctaacactcacaacaaaactaactaatactaacatctcagacgctcaggaaatagaaaccgtctgaactatcctgcccgccatcatcctagtcctcatcgccctcccatccctacgcatcctttacataacagacgaggtcaacgatccctcccttaccatcaaatcaattggccaccaatggtactgaacctacgagtacaccgactacggcggact

SEQ ID NO: 558 - DNA, 239 bp, Homo sapiens
  source 1..239: mol_type = genomic DNA
cgggcgaggcctatattacggatcatttctctactcagaaacctgaaacatcggcattatcctcctgcttgcaactatagcaacagccttcataggctatgtcctcccgtgaggccaaatatcattctgaggggccacagtaattacaaacttactatccgccatcccatacattgggacagacctagttcaatgaatctgaggaggctactcagtagacagtcccaccctcacacgat

SEQ ID NO: 559 - DNA, 260 bp, Mus musculus
  source 1..260: mol_type = genomic DNA
acaaaggtttggtcctggccttataattaattagaggtaaaattacacatgcaaacctccatagaccggtgtaaaatcccttaaacatttacttaaaatttaaggagagggtatcaagcacattaaaatagcttaagacaccttgcctagccacacccccacgggactcagcagtgataaatattaagcaataaacgaaagtttgactaagttatacctcttagggttggtaaatttcgtgccagccaccgcggtcatac

SEQ ID NO: 560 - DNA, 249 bp, Mus musculus
  source 1..249: mol_type = genomic DNA
gtcatttataatacacgacagctaagacccaaactgggattagataccccactatgcttagccataaacctaaataattaaatttaacaaaactatttgccagagaactactagccatagcttaaaactcaaaggacttggcggtactttatatccatctagaggagcctgttctataatcgataaaccccgctctacctcaccatctcttgctaattcagcctatataccgccatcttcagcaaaccc

SEQ ID NO: 561 - DNA, 261 bp, Mus musculus
  source 1..261: mol_type = genomic DNA
gcctacacccagaagatttcatgaccaatgaacactctgaactaatcctagccctagccctacacaaatataattatactattatataaatcaaaacatttatcctactaaaagtattggagaaagaaattcgtacatctaggagctatagaactagtaccgcaagggaaagatgaaagactaattaaaagtaagaacaagcaaagattaaaccttgtaccttttgcataatgaactaactagaaaacttctaactaaaag

SEQ ID NO: 562 - DNA, 278 bp, Mus musculus
  source 1..278: mol_type = genomic DNA
ctttaatcagtgaaattgacctttcagtgaagaggctgaaatataataataagacgagaagaccctatggagcttaaattatataacttatctatttaatttattaaacctaatggcccaaaaactatagtataagtttgaaatttcggttggggtgacctcggagaataaaaaatcctccgaatgattataacctagacttacaagtcaaagtaaaatcaacatatcttattgacccagatatattttgatcaacggaccaagttaccctagggata

SEQ ID NO: 563 - DNA, 286 bp, Mus musculus
  source 1..286: mol_type = genomic DNA
acaaacacttattacaacccaagaacacatatgattacttctgccagcctgacccatagccataatatgatttatctcaaccctagcagaaacaaaccgggcccccttcgacctgacagaaggagaatcagaattagtatcagggtttaacgtagaatacgcagccggcccattcgcgttattctttatagcagagtacactaacattattctaataaacgccctaacaactattatcttcctaggacccctatactatatcaatttaccagaactctactcaact

SEQ ID NO: 564 - DNA, 265 bp, Mus musculus
  source 1..265: mol_type = genomic DNA
caactgtctaattatagcaacactcatagcaataatagctctactaaacctattcttttatactcgcctaatttattccacttcactaacaatatttccaaccaacaataactcaaaaataataactcaccaaacaaaaactaaacccaacctaatattttccaccctagctatcataagcacaataaccctacccctagccccccaactaattacctagaagtttaggatatactagtccgcgagccttcaaagccctaagaaa

SEQ ID NO: 565 - DNA, 264 bp, Mus musculus
  source 1..264: mol_type = genomic DNA
taatataagtttttgactcctaccaccatcatttctccttctcctagcatcatcaatagtagaagcaggagcaggaacaggatgaacagtctacccacctctagccggaaatctagcccatgcaggagcatcagtagacctaacaattttctcccttcatttagctggagtgtcatctattttaggtgcaattaattttattaccactattatcaacatgaaacccccagccataacacagtatcaaactccactatttgtctg

SEQ ID NO: 566 - DNA, 272 bp, Mus musculus
  source 1..272: mol_type = genomic DNA
cttcaacaaatttagaatgacttcatggctgccctccaccatatcacacattcgaggaaccaacctatgtaaaagtaaaataagaaaggaaggaatcgaaccccctaaaattggtttcaagccaatctcatatcctatatgtctttctcaataagatattagtaaaatcaattacataactttgtcaaagttaaattatagatcaataatctatatatcttatatggcctacccattccaacttggtctacaagacgccacatcccctatta

SEQ ID NO: 567 - DNA, 241 bp, Mus musculus
  source 1..241: mol_type = genomic DNA
tcatcatagccttatagaaggtaaacgaaaccacataaatcaagccctactaattaccattatactaggactttacttcaccatcctccaagcttcagaatactttgaaacatcattctccatttcagatggtatctatggttctacattcttcatggctactggattccatggactccatgtaattattggatcaacattccttattgtttgcctactacgacaactaaaatttcacttc

SEQ ID NO: 568 - DNA, 275 bp, Mus musculus
  source 1..275: mol_type = genomic DNA
accatagccttctcactatcacttctagggacacttatatttcgctctcacctaatatccacattactatgcctggaaggcatagtattatccttatttattataacttcagtaacttccctaaactccaactccataagctccataccaatccccatcaccatcttagttttcgcagcctgcgaagcagctgtaggactagccctactagtaaaagtttcaaacacgtacggaacagattacgtccaaaatctcaacctactacaatgctaaaa

SEQ ID NO: 569 - DNA, 254 bp, Mus musculus
  source 1..254: mol_type = genomic DNA
ccttagacgcttcatgatctaacaacttactatggttggcatgcataatagcatttcttattaaaataccattatatggagttcacctatgactaccaaaagcccatgttgaagctccaattgctgggtcaataattctagcagctattcttctaaaattaggtagttacggaataattcgcatctccattattctagacccactaacaaaatatatagcataccccttcatccttctctccctatgaggaata

SEQ ID NO: 570 - DNA, 274 bp, Mus musculus
  source 1..274: mol_type = genomic DNA
cttttcattggctgagaaggggtgggaattatatctttcctactaattggatgatggtacggacgaacagacgcaaatactgcagccctacaagcaatcctctataaccgcatcggagacatcggattcattttagctatagtttgattttccctaaacataaactcatgagaacttcaacagattatattctccaacaacaacgacaatctaattccacttataggcctattaatcgcagctacaggaaaatcagcacaatttggcctccacc

SEQ ID NO: 571 - DNA, 225 bp, Homo sapiens
  source 1..225: mol_type = genomic DNA
cctagggttggccaatctactcccaggagcagggagggcaggagccagggctgggcatagaagtcagggcagagccatctattgcttacatttgcttctgacacaactgtgttcactagcaacctcaaacagacaccatggtgcatctgactcctgaggagaagtctgccgttactgccctgtggggcaaggtaaaccagctttacactccctcacactgatcac

SEQ ID NO: 572 - DNA, 223 bp, Homo sapiens
  source 1..223: mol_type = genomic DNA
tggtgctgtgggtggggtcctctctattttgtcctctgtaggtgccttttcctccgtacccctcttcagaccaggcctctgagatttgcctcctactcaggacacccagggttgggtggaggccacggctctgcccacacccccctgccccttggccaacaccctgtcttcgcctgtggctctcctgcaccctggggcctgggctccagaatcagcctccctt

SEQ ID NO: 573 - DNA, 233 bp, Homo sapiens
  source 1..233: mol_type = genomic DNA
atggtttcacaacagcacatggtgaaaaggttaaaaaaaaaaagtcaccctatacattgtcaatgaaattcatatgaggagctataacctgacactcctactctccccatcagggaggaatgagtggaggcctagacagcgaccagaactcccaaccccaaccaggagtaaccaggaacatccctcactcaggagtcaatgggggccagggatggaacactcttttacactca

SEQ ID NO: 574 - DNA, 226 bp, Homo sapiens
  source 1..226: mol_type = genomic DNA
tgaagtctggagactcactgaatttgttctgggtatgggggaggttacatgggtttaatggtttttaaatttatttggggggaataagggttgtctttgggtacactacagcgatggctattgaggagtatcctgaggcatgggggtcaggggttgaggtcttggtaagtgttttagtggggttagcgattctggtacagtggcttgtgcctgtaatcccagcact

SEQ ID NO: 575 - DNA, 281 bp, Homo sapiens
  source 1..281: mol_type = genomic DNA
tctagcttatattcactaatgggaaaacgtattctaataaactagcactgactcattacatgaatagatctaagccatcaagtgacagacagaaaattatgtgctgttgtgaaagacaaggaagggcaggagaagatagtgagaagtcagtgaggctccaagcaaaattatgctgcatttgatatatttctccacatgtaaatacacaacaggtgtagctgaaacttgcttgctagtcatgtaaaacattgcactatgaaactttacttacaattagagac

SEQ ID NO: 576 - DNA, 59 bp, Synthetic construct
  source 1..59: mol_type = other DNA
acactctttccctacacgacgctcttccgatctnnnnctttacagtgaaatgccccaac

SEQ ID NO: 577 - DNA, 48 bp, Synthetic construct
  source 1..48: mol_type = other DNA
tggagttcagacgtgtgctcttccgatctgggggcaatgaatgaagcg

SEQ ID NO: 578 - DNA, 56 bp, Synthetic construct
  source 1..56: mol_type = other DNA
acactctttccctacacgacgctcttccgatctnnnncgggtccatcatccacaac

SEQ ID NO: 579 - DNA, 52 bp, Synthetic construct
  source 1..52: mol_type = other DNA
tggagttcagacgtgtgctcttccgatctagagtaatagatagggctcaggc

SEQ ID NO: 580 - DNA, 59 bp, Synthetic construct
  source 1..59: mol_type = other DNA
acactctttccctacacgacgctcttccgatctnnnnaaagtttaccacaaccaccacc

SEQ ID NO: 581 - DNA, 55 bp, Synthetic construct
  source 1..55: mol_type = other DNA
tggagttcagacgtgtgctcttccgatcttgggggaggttatatgggtttaatag

SEQ ID NO: 582 - DNA, 57 bp, Synthetic construct
  source 1..57: mol_type = other DNA
acactctttccctacacgacgctcttccgatctnnnncctactcctgctcgcatctg

SEQ ID NO: 583 - DNA, 50 bp, Synthetic construct
  source 1..50: mol_type = other DNA
tggagttcagacgtgtgctcttccgatcttggtattgggttatggcaggg

SEQ ID NO: 584 - DNA, 61 bp, Synthetic construct
  source 1..61: mol_type = other DNA
acactctttccctacacgacgctcttccgatctnnnnggctatatacaactacgcaaaggc

SEQ ID NO: 585 - DNA, 54 bp, Synthetic construct
  source 1..54: mol_type = other DNA
tggagttcagacgtgtgctcttccgatctataggaggcctaggttgaggttgac

SEQ ID NO: 586 - DNA, 60 bp, Synthetic construct
  source 1..60: mol_type = other DNA
acactctttccctacacgacgctcttccgatctnnnnaatcggaggctttggcaactgac

SEQ ID NO: 587 - DNA, 53 bp, Synthetic construct
  source 1..53: mol_type = other DNA
tggagttcagacgtgtgctcttccgatctggttaggtctacggaggctccagg

SEQ ID NO: 588 - DNA, 60 bp, Synthetic construct
  source 1..60: mol_type = other DNA
acactctttccctacacgacgctcttccgatctnnnnataccaaacgcccctcttcgtct

SEQ ID NO: 589 - DNA, 55 bp, Synthetic construct
  source 1..55: mol_type = other DNA
tggagttcagacgtgtgctcttccgatctccgaagcctggtaggataagaatata

SEQ ID NO: 590 - DNA, 62 bp, Synthetic construct
  source 1..62: mol_type = other DNA
acactctttccctacacgacgctcttccgatctnnnngcatcctttacataacagacgaggt

SEQ ID NO: 591 - DNA, 57 bp, Synthetic construct
  source 1..57: mol_type = other DNA
tggagttcagacgtgtgctcttccgatctttatacgaatgggggcttcaatcgggag

SEQ ID NO: 592 - DNA, 63 bp, Synthetic construct
  source 1..63: mol_type = other DNA
acactctttccctacacgacgctcttccgatctnnnncccataatcatacaaagcccccgcac

SEQ ID NO: 593 - DNA, 53 bp, Synthetic construct
  source 1..53: mol_type = other DNA
tggagttcagacgtgtgctcttccgatctgctattgaggagtatcctgaggca

SEQ ID NO: 594 - DNA, 64 bp, Synthetic construct
  source 1..64: mol_type = other DNA
acactctttccctacacgacgctcttccgatctnnnngacttcaaactctactcccactaatag

SEQ ID NO: 595 - DNA, 53 bp, Synthetic construct
  source 1..53: mol_type = other DNA
tggagttcagacgtgtgctcttccgatctgttgtggtaaatatgtagagggag

SEQ ID NO: 596 - DNA, 58 bp, Synthetic construct
  source 1..58: mol_type = other DNA
acactctttccctacacgacgctcttccgatctnnnnggagcaaaccacagtttcatg

SEQ ID NO: 597 - DNA, 54 bp, Synthetic construct
  source 1..54: mol_type = other DNA
tggagttcagacgtgtgctcttccgatcttgaggaatagtgtaaggagtatggg

SEQ ID NO: 598 - DNA, 63 bp, Synthetic construct
  source 1..63: mol_type = other DNA
acactctttccctacacgacgctcttccgatctnnnngatctaaccatagctttatgcccatt

SEQ ID NO: 599 - DNA, 56 bp, Synthetic construct
  source 1..56: mol_type = other DNA
tggagttcagacgtgtgctcttccgatctagggtaattattgatgagataattgtg

SEQ ID NO: 600 - DNA, 67 bp, Synthetic construct
  source 1..67: mol_type = other DNA
acactctttccctacacgacgctcttccgatctnnnnctagcctatcagtttactccattctatgat

SEQ ID NO: 601 - DNA, 59 bp, Synthetic construct
  source 1..59: mol_type = other DNA
tggagttcagacgtgtgctcttccgatctgataaatcatattatggctatgggtcaggc

SEQ ID NO: 602 - DNA, 63 bp, Synthetic construct
  source 1..63: mol_type = other DNA
acactctttccctacacgacgctcttccgatctnnnnaccatcgctcttctactatgaacccc

SEQ ID NO: 603 - DNA, 57 bp, Synthetic construct
  source 1..57: mol_type = other DNA
tggagttcagacgtgtgctcttccgatctgttaaaggagccacttattagtaatgtt

SEQ ID NO: 604 - DNA, 63 bp, Synthetic construct
  source 1..63: mol_type = other DNA
acactctttccctacacgacgctcttccgatctnnnngggccattatcgaagaattcacaaaa

SEQ ID NO: 605 - DNA, 58 bp, Synthetic construct
  source 1..58: mol_type = other DNA
tggagttcagacgtgtgctcttccgatctgggagataggtaggagtagcgtggtaagg

SEQ ID NO: 606 - DNA, 63 bp, Synthetic construct
  source 1..63: mol_type = other DNA
acactctttccctacacgacgctcttccgatctnnnntcataaccctcaacacccactccctc

SEQ ID NO: 607 - DNA, 57 bp, Synthetic construct
  source 1..57: mol_type = other DNA
tggagttcagacgtgtgctcttccgatcttgttttttggaaagtcatgtcagtggta

SEQ ID NO: 608 - DNA, 64 bp, Synthetic construct
  source 1..64: mol_type = other DNA
acactctttccctacacgacgctcttccgatctnnnncttatccagtgaaccactatcacgaaa

SEQ ID NO: 609 - DNA, 56 bp, Synthetic construct
  source 1..56: mol_type = other DNA
tggagttcagacgtgtgctcttccgatctgagcctactagggtgtagaataggaag

SEQ ID NO: 610 - DNA, 67 bp, Synthetic construct
  source 1..67: mol_type = other DNA
acactctttccctacacgacgctcttccgatctnnnnccttcttgctcatcagttgatgatacgccc

SEQ ID NO: 611 - DNA, 55 bp, Synthetic construct
  source 1..55: mol_type = other DNA
tggagttcagacgtgtgctcttccgatctctaattgggctgatttgcctgctgct

SEQ ID NO: 612 - DNA, 66 bp, Synthetic construct
  source 1..66: mol_type = other DNA
acactctttccctacacgacgctcttccgatctnnnngctattacctaaaacaatttcacagcacc

SEQ ID NO: 613 - DNA, 59 bp, Synthetic construct
  source 1..59: mol_type = other DNA
tggagttcagacgtgtgctcttccgatctggctttgtatgattatgggcgttgattagt

SEQ ID NO: 614 - DNA, 63 bp, Synthetic construct
  source 1..63: mol_type = other DNA
acactctttccctacacgacgctcttccgatctnnnncatcataatcggaggctttggcaact

SEQ ID NO: 615 - DNA, 54 bp, Synthetic construct
  source 1..54: mol_type = other DNA
tggagttcagacgtgtgctcttccgatctacggaggctccagggtgggagtagt

SEQ ID NO: 616 - DNA, 65 bp, Synthetic construct
  source 1..65: mol_type = other DNA
acactctttccctacacgacgctcttccgatctnnnntgcccttttcctaacactcacaacaaaa

SEQ ID NO: 617 - DNA, 60 bp, Synthetic construct
  source 1..60: mol_type = other DNA
tggagttcagacgtgtgctcttccgatctagtccgccgtagtcggtgtactcgtaggttc

SEQ ID NO: 618 - DNA, 64 bp, Synthetic construct
  source 1..64: mol_type = other DNA
acactctttccctacacgacgctcttccgatctnnnncgggcgaggcctatattacggatcatt

SEQ ID NO: 619 - DNA, 57 bp, Synthetic construct
  source 1..57: mol_type = other DNA
tggagttcagacgtgtgctcttccgatctatcgtgtgagggtgggactgtctactga

SEQ ID NO: 620 - DNA, 65 bp, Synthetic construct
  source 1..65: mol_type = other DNA
acactctttccctacacgacgctcttccgatctnnnnacaaaggtttggtcctggccttataatt

SEQ ID NO: 621 - DNA, 56 bp, Synthetic construct
  source 1..56: mol_type = other DNA
tggagttcagacgtgtgctcttccgatctgtatgaccgcggtggctggcacgaaat

SEQ ID NO: 622 - DNA, 67 bp, Synthetic construct
  source 1..67: mol_type = other DNA
acactctttccctacacgacgctcttccgatctnnnngtcatttataatacacgacagctaagaccc

SEQ ID NO: 623 - DNA, 57 bp, Synthetic construct
  source 1..57: mol_type = other DNA
tggagttcagacgtgtgctcttccgatctgggtttgctgaagatggcggtatatagg

SEQ ID NO: 624 - DNA, 66 bp, Synthetic construct
  source 1..66: mol_type = other DNA
acactctttccctacacgacgctcttccgatctnnnngcctacacccagaagatttcatgaccaat

SEQ ID NO: 625 - DNA, 59 bp, Synthetic construct
  source 1..59: mol_type = other DNA
tggagttcagacgtgtgctcttccgatctcttttagttagaagttttctagttagttca

SEQ ID NO: 626 - DNA, 64 bp, Synthetic construct
  source 1..64: mol_type = other DNA
acactctttccctacacgacgctcttccgatctnnnnctttaatcagtgaaattgacctttcag

SEQ ID NO: 627 - DNA, 57 bp, Synthetic construct
  source 1..57: mol_type = other DNA
tggagttcagacgtgtgctcttccgatcttatccctagggtaacttggtccgttgat

SEQ ID NO: 628 - DNA, 67 bp, Synthetic construct
  source 1..67: mol_type = other DNA
acactctttccctacacgacgctcttccgatctnnnnacaaacacttattacaacccaagaacacat

SEQ ID NO: 629 - DNA, 57 bp, Synthetic construct
  source 1..57: mol_type = other DNA
tggagttcagacgtgtgctcttccgatctagttgagtagagttctggtaaattgata

SEQ ID NO: 630 - DNA, 67 bp, Synthetic construct
  source 1..67: mol_type = other DNA
acactctttccctacacgacgctcttccgatctnnnncaactgtctaattatagcaacactcatagc

SEQ ID NO: 631 - DNA, 58 bp, Synthetic construct
  source 1..58: mol_type = other DNA
tggagttcagacgtgtgctcttccgatcttttcttagggctttgaaggctcgcggact

SEQ ID NO: 632 - DNA, 67 bp, Synthetic construct
  source 1..67: mol_type = other DNA
acactctttccctacacgacgctcttccgatctnnnntaatataagtttttgactcctaccaccatc

SEQ ID NO: 633 - DNA, 59 bp, Synthetic construct
  source 1..59: mol_type = other DNA
tggagttcagacgtgtgctcttccgatctcagacaaatagtggagtttgatactgtgtt

SEQ ID NO: 634 - DNA, 66 bp, Synthetic construct
  source 1..66: mol_type = other DNA
acactctttccctacacgacgctcttccgatctnnnncttcaacaaatttagaatgacttcatggc

SEQ ID NO: 635 - DNA, 59 bp, Synthetic construct
  source 1..59: mol_type = other DNA
tggagttcagacgtgtgctcttccgatcttaataggggatgtggcgtcttgtagaccaa

SEQ ID NO: 636 - DNA, 67 bp, Synthetic construct
  source 1..67: mol_type = other DNA
acactctttccctacacgacgctcttccgatctnnnntcatcatagccttatagaaggtaaacgaaa

SEQ ID NO: 637 - DNA, 59 bp, Synthetic construct
  source 1..59: mol_type = other DNA
tggagttcagacgtgtgctcttccgatctgaagtgaaattttagttgtcgtagtaggca

SEQ ID NO: 638 - DNA, 66 bp, Synthetic construct
  source 1..66: mol_type = other DNA
acactctttccctacacgacgctcttccgatctnnnnaccatagccttctcactatcacttctagg

SEQ ID NO: 639 - DNA, 60 bp, Synthetic construct
  source 1..60: mol_type = other DNA
tggagttcagacgtgtgctcttccgatctttttagcattgtagtaggttgagattttgga

SEQ ID NO: 640 - DNA, 67 bp, Synthetic construct
  source 1..67: mol_type = other DNA
acactctttccctacacgacgctcttccgatctnnnnccttagacgcttcatgatctaacaacttac

SEQ ID NO: 641 - DNA, 58 bp, Synthetic construct
  source 1..58: mol_type = other DNA
tggagttcagacgtgtgctcttccgatcttattcctcatagggagagaaggatgaagg

SEQ ID NO: 642 - DNA, 66 bp, Synthetic construct
  source 1..66: mol_type = other DNA
acactctttccctacacgacgctcttccgatctnnnncttttcattggctgagaaggggtgggaat

SEQ ID NO: 643 - DNA, 59 bp, Synthetic construct
  source 1..59: mol_type = other DNA
tggagttcagacgtgtgctcttccgatctggtggaggccaaattgtgctgattttcctg

SEQ ID NO: 644 - DNA, 66 bp, Synthetic construct
  source 1..66: mol_type = other DNA
acactctttccctacacgacgctcttccgatctnnnngtgatcagtgtgagggagtgtaaagctgg

SEQ ID NO: 645 - DNA, 53 bp, Synthetic construct
  source 1..53: mol_type = other DNA
tggagttcagacgtgtgctcttccgatctcctagggttggccaatctactccc

SEQ ID NO: 646 - DNA, 67 bp, Synthetic construct
  source 1..67: mol_type = other DNA
acactctttccctacacgacgctcttccgatctnnnntggtgctgtgggtggggtcctctctatttt

SEQ ID NO: 647 - DNA, 57 bp, Synthetic construct
  source 1..57: mol_type = other DNA
tggagttcagacgtgtgctcttccgatctaagggaggctgattctggagcccaggcc

SEQ ID NO: 648 - DNA, 69 bp, Synthetic construct
  source 1..69: mol_type = other DNA
acactctttccctacacgacgctcttccgatctnnnnatggtttcacaacagcacatggtgaaaaggtt

SEQ ID NO: 649 - DNA, 59 bp, Synthetic construct
  source 1..59: mol_type = other DNA
tggagttcagacgtgtgctcttccgatcttgagtgtaaaagagtgttccatccctggcc

SEQ ID NO: 650 - DNA, 67 bp, Synthetic construct
  source 1..67: mol_type = other DNA
acactctttccctacacgacgctcttccgatctnnnntgaagtctggagactcactgaatttgttct

SEQ ID NO: 651 - DNA, 58 bp, Synthetic construct
  source 1..58: mol_type = other DNA
tggagttcagacgtgtgctcttccgatctagtgctgggattacaggcacaagccactg

SEQ ID NO: 652 - DNA, 69 bp, Synthetic construct
  source 1..69: mol_type = other DNA
acactctttccctacacgacgctcttccgatctnnnntctagcttatattcactaatgggaaaacgtat

SEQ ID NO: 653 - DNA, 61 bp, Synthetic construct
  source 1..61: mol_type = other DNA
tggagttcagacgtgtgctcttccgatctgtctctaattgtaagtaaagtttcatagtgca

SEQ ID NO: 654 - DNA, 15 bp, Homo sapiens
  source 1..15: mol_type = genomic DNA
aggtaggtggtagtt

SEQ ID NO: 655 - DNA, 15 bp, Homo sapiens
  source 1..15: mol_type = genomic DNA
caccaaagcccataa

SEQ ID NO: 656 - DNA, 15 bp, Homo sapiens
  source 1..15: mol_type = genomic DNA
agcccataaaaataa

SEQ ID NO: 657 - DNA, 15 bp, Homo sapiens
  source 1..15: mol_type = genomic DNA
ttgaagtgagaggta

SEQ ID NO: 658 - DNA, 15 bp, Homo sapiens
  source 1..15: mol_type = genomic DNA
aagtgagaggtatgg

SEQ ID NO: 659 - DNA, 15 bp, Homo sapiens
  source 1..15: mol_type = genomic DNA
ccattggcagcctag

SEQ ID NO: 660 - DNA, 12 bp, Homo sapiens
  source 1..12: mol_type = genomic DNA
taggtggtagtt

SEQ ID NO: 661 - DNA, 12 bp, Homo sapiens
  source 1..12: mol_type = genomic DNA
aggtaggtggta

SEQ ID NO: 662 - DNA, 12 bp, Homo sapiens
  source 1..12: mol_type = genomic DNA
caaagcccataa

SEQ ID NO: 663 - DNA, 12 bp, Homo sapiens
  source 1..12: mol_type = genomic DNA
caccaaagccca

SEQ ID NO: 664 - DNA, 12 bp, Homo sapiens
  source 1..12: mol_type = genomic DNA
ccataaaaataa

SEQ ID NO: 665 - DNA, 12 bp, Homo sapiens
  source 1..12: mol_type = genomic DNA
agcccataaaaa

SEQ ID NO: 666 - DNA, 12 bp, Homo sapiens
  source 1..12: mol_type = genomic DNA
gattgagtaaac

SEQ ID NO: 667 - DNA, 12 bp, Homo sapiens
  source 1..12: mol_type = genomic DNA
gatgctcaccct

SEQ ID NO: 668 - DNA, 12 bp, Homo sapiens
  source 1..12: mol_type = genomic DNA
gattaggcgtag

SEQ ID NO: 669 - DNA, 12 bp, Homo sapiens
  source 1..12: mol_type = genomic DNA
ggagtagtgtga

SEQ ID NO: 670 - DNA, 12 bp, Homo sapiens
  source 1..12: mol_type = genomic DNA
gactagtagggc

SEQ ID NO: 671 - DNA, 12 bp, Homo sapiens
  source 1..12: mol_type = genomic DNA
aacacatatggc

SEQ ID NO: 672 - DNA, 12 bp, Homo sapiens
  source 1..12: mol_type = genomic DNA
gatataaaatat

SEQ ID NO: 673 - DNA, 12 bp, Homo sapiens
  source 1..12: mol_type = genomic DNA
aaccacacttat

SEQ ID NO: 674 - DNA, 12 bp, Homo sapiens
  source 1..12: mol_type = genomic DNA
tgaaaccgatat

SEQ ID NO: 675 - DNA, 12 bp, Homo sapiens
  source 1..12: mol_type = genomic DNA
aatcatgctaag

SEQ ID NO: 676 - DNA, 12 bp, Homo sapiens
  source 1..12: mol_type = genomic DNA
gtgggaagaaga

SEQ ID NO: 677 - DNA, 12 bp, Homo sapiens
  source 1..12: mol_type = genomic DNA
taggagtagggt

SEQ ID NO: 678 - DNA, 12 bp, Homo sapiens
  source 1..12: mol_type = genomic DNA
gtaagagtcaga

SEQ ID NO: 679 - DNA, 12 bp, Homo sapiens
  source 1..12: mol_type = genomic DNA
cgagcaggagta

SEQ ID NO: 680 - DNA, 12 bp, Homo sapiens
  source 1..12: mol_type = genomic DNA
taggatgatggc

SEQ ID NO: 681 - DNA, 12 bp, Homo sapiens
  source 1..12: mol_type = genomic DNA
tagggatgggag

SEQ ID NO: 682 - DNA, 12 bp, Homo sapiens
  source 1..12: mol_type = genomic DNA
atagcctatgaa

SEQ ID NO: 683 - DNA, 12 bp, Homo sapiens
  source 1..12: mol_type = genomic DNA
tgaggccaaata

SEQ ID NO: 684 - DNA, 12 bp, Homo sapiens
  source 1..12: mol_type = genomic DNA
taagttaaagat

SEQ ID NO: 685 - DNA, 12 bp, Homo sapiens
  source 1..12: mol_type = genomic DNA
aaagaggtgttg

SEQ ID NO: 686 - DNA, 15 bp, Homo sapiens
  source 1..15: mol_type = genomic DNA
tgtaaagaggtgttg

SEQ ID NO: 687 - DNA, 12 bp, Homo sapiens
  source 1..12: mol_type = genomic DNA
gtagatgtggcg

SEQ ID NO: 688 - DNA, 12 bp, Homo sapiens
  source 1..12: mol_type = genomic DNA
gatgtggcgggt

SEQ ID NO: 689 - DNA, 15 bp, Homo sapiens
  source 1..15: mol_type = genomic DNA
gtagatgtggcgggt

SEQ ID NO: 690 - DNA, 12 bp, Homo sapiens
  source 1..12: mol_type = genomic DNA
gatgtagagggt

SEQ ID NO: 691 - DNA, 12 bp, Homo sapiens
  source 1..12: mol_type = genomic DNA
ggtgatgtagag

SEQ ID NO: 692 - DNA, 12 bp, Homo sapiens
  source 1..12: mol_type = genomic DNA
ggcggtgatgta

SEQ ID NO: 693 - DNA, 15 bp, Homo sapiens
  source 1..15: mol_type = genomic DNA
ggtgatgtagagggt

SEQ ID NO: 694 - DNA, 15 bp, Homo sapiens
  source 1..15: mol_type = genomic DNA
ggcggtgatgtagag

SEQ ID NO: 695 - DNA, 12 bp, Homo sapiens
  source 1..12: mol_type = genomic DNA
gcaggagtagga

SEQ ID NO: 696 - DNA, 12 bp, Homo sapiens
  source 1..12: mol_type = genomic DNA
ggagtaggagag

SEQ ID NO: 697 - DNA, 15 bp, Homo sapiens
  source 1..15: mol_type = genomic DNA
gcaggagtaggagag

SEQ ID NO: 698 - DNA, 12 bp, Homo sapiens
  source 1..12: mol_type = genomic DNA
gtggaggccgga

SEQ ID NO: 699 - DNA, 12 bp, Homo sapiens
  source 1..12: mol_type = genomic DNA
gaggccggagca

SEQ ID NO: 700 - DNA, 12 bp, Homo sapiens
  source 1..12: mol_type = genomic DNA
gccggagcagga

SEQ ID NO: 701 - DNA, 15 bp, Homo sapiens
  source 1..15: mol_type = genomic DNA
gtggaggccggagca

SEQ ID NO: 702 - DNA, 15 bp, Homo sapiens
  source 1..15: mol_type = genomic DNA
gaggccggagcagga

SEQ ID NO: 703 - DNA, 12 bp, Homo sapiens
  source 1..12: mol_type = genomic DNA
ggcggggtcgaa

SEQ ID NO: 704 - DNA, 12 bp, Homo sapiens
  source 1..12: mol_type = genomic DNA
ggggtcgaagaa

SEQ ID NO: 705 - DNA, 12 bp, Homo sapiens
  source 1..12: mol_type = genomic DNA
gtcgaagaaggt

SEQ ID NO: 706 - DNA, 12 bp, Homo sapiens
  source 1..12: mol_type = genomic DNA
gaagaaggtggt

SEQ ID NO: 707 - DNA, 12 bp, Homo sapiens
  source 1..12: mol_type = genomic DNA
gaaggtggtgtt

SEQ ID NO: 708 - DNA, 12 bp, Homo sapiens
  source 1..12: mol_type = genomic DNA
ggtggtgttgag

SEQ ID NO: 709 - DNA, 12 bp, Homo sapiens
  source 1..12: mol_type = genomic DNA
ggtgttgaggtt

SEQ ID NO: 710 - DNA, 12 bp, Homo sapiens
  source 1..12: mol_type = genomic DNA
gttgaggttgcg

SEQ ID NO: 711 - DNA, 12 bp, Homo sapiens
  source 1..12: mol_type = genomic DNA
gaggttgcggtc

SEQ ID NO: 712 - DNA, 15 bp, Homo sapiens
  source 1..15: mol_type = genomic DNA
ggcggggtcgaagaa

SEQ ID NO: 713 - DNA, 15 bp, Homo sapiens
  source 1..15: mol_type = genomic DNA
ggggtcgaagaaggt

SEQ ID NO: 714 - DNA, 15 bp, Homo sapiens
  source 1..15: mol_type = genomic DNA
gtcgaagaaggtggt

SEQ ID NO: 715 - DNA, 15 bp, Homo sapiens
  source 1..15: mol_type = genomic DNA
gaagaaggtggtgtt

SEQ ID NO: 716 - DNA, 15 bp, Homo sapiens
  source 1..15: mol_type = genomic DNA
gaaggtggtgttgag

SEQ ID NO: 717 - DNA, 15 bp, Homo sapiens
  source 1..15: mol_type = genomic DNA
ggtggtgttgaggtt

SEQ ID NO: 718 - DNA, 15 bp, Homo sapiens
  source 1..15: mol_type = genomic DNA
ggtgttgaggttgcg

SEQ ID NO: 719 - DNA, 15 bp, Homo sapiens
  source 1..15: mol_type = genomic DNA
gttgaggttgcggtc

SEQ ID NO: 720 - DNA, 12 bp, Homo sapiens
  source 1..12: mol_type = genomic DNA
gccggaggagga

SEQ ID NO: 721 - DNA, 12 bp, Homo sapiens
  source 1..12: mol_type = genomic DNA
ggaggaggagac

SEQ ID NO: 722 - DNA, 15 bp, Homo sapiens
  source 1..15: mol_type = genomic DNA
gccggaggaggagac

SEQ ID NO: 723 - DNA, 12 bp, Homo sapiens
  source 1..12: mol_type = genomic DNA
gccgtagtcggt

SEQ ID NO: 724 - DNA, 12 bp, Homo sapiens
  source 1..12: mol_type = genomic DNA
gtagtcggtgta

SEQ ID NO: 725 - DNA, 15 bp, Homo sapiens
  source 1..15: mol_type = genomic DNA
gccgtagtcggtgta

SEQ ID NO: 726 - DNA, 12 bp, Homo sapiens
  source 1..12: mol_type = genomic DNA
ggagttgaagat

SEQ ID NO: 727 - DNA, 12 bp, Homo sapiens
  source 1..12: mol_type = genomic DNA
gtaggagttgaa

SEQ ID NO: 728 - DNA, 15 bp, Homo sapiens
  source 1..15: mol_type = genomic DNA
gtaggagttgaagat

SEQ ID NO: 729 - DNA, 12 bp, Homo sapiens
  source 1..12: mol_type = genomic DNA
gatggggtggtg

SEQ ID NO: 730 - DNA, 12 bp, Homo sapiens
  source 1..12: mol_type = genomic DNA
ggggtggtggtt

SEQ ID NO: 731 - DNA, 12 bp, Homo sapiens
  source 1..12: mol_type = genomic DNA
gtggtggttgtg

SEQ ID NO: 732 - DNA, 12 bp, Homo sapiens
  source 1..12: mol_type = genomic DNA
gtggttgtggta

SEQ ID NO: 733 - DNA, 15 bp, Homo sapiens
  source 1..15: mol_type = genomic DNA
gatggggtggtggtt

SEQ ID NO: 734 - DNA, 15 bp, Homo sapiens
  source 1..15: mol_type = genomic DNA
ggggtggtggttgtg

SEQ ID NO: 735 - DNA, 15 bp, Homo sapiens
  source 1..15: mol_type = genomic DNA
gtggtggttgtggta

SEQ ID NO: 736 - DNA, 12 bp, Homo sapiens
  source 1..12: mol_type = genomic DNA
gctgtgggtgaa

SEQ ID NO: 737 - DNA, 12 bp, Homo sapiens
  source 1..12: mol_type = genomic DNA
ggtgctgtgggt

SEQ ID NO: 738 - DNA, 15 bp, Homo sapiens
  source 1..15: mol_type = genomic DNA
ggtgctgtgggtgaa

SEQ ID NO: 739 - DNA, 12 bp, Mus musculus
  source 1..12: mol_type = genomic DNA
gttaaagttaga

SEQ ID NO: 740 - DNA, 15 bp, Mus musculus
  source 1..15: mol_type = genomic DNA
taagttaaagttaga

SEQ ID NO: 741 - DNA, 12 bp, Mus musculus
  source 1..12: mol_type = genomic DNA
actatggagatt

SEQ ID NO: 742 - DNA, 12 bp, Mus musculus
  source 1..12: mol_type = genomic DNA
cactatggagat

SEQ ID NO: 743 - DNA, 15 bp, Mus musculus
  source 1..15: mol_type = genomic DNA
tatcactatggagat

SEQ ID NO: 744 - DNA, 18 bp, Mus musculus
  source 1..18: mol_type = genomic DNA
gcatatcactatggagat

SEQ ID NO: 745 - DNA, 12 bp, Mus musculus
  source 1..12: mol_type = genomic DNA
gtttgggctacg

SEQ ID NO: 746 - DNA, 15 bp, Mus musculus
  source 1..15: mol_type = genomic DNA
gtttgggctacggct

SEQ ID NO: 747 - DNA, 15 bp, Mus musculus
  source 1..15: mol_type = genomic DNA
accatagctattatc

SEQ ID NO: 748 - DNA, 18 bp, Mus musculus
  source 1..18: mol_type = genomic DNA
accatagctattatcctt

SEQ ID NO: 749 - DNA, 12 bp, Mus musculus
  source 1..12: mol_type = genomic DNA
atagctatggtt

SEQ ID NO: 750 - DNA, 15 bp, Mus musculus
  source 1..15: mol_type = genomic DNA
ataatagctatggtt

SEQ ID NO: 751 - DNA, 18 bp, Mus musculus
  source 1..18: mol_type = genomic DNA
aggataatagctatggtt

SEQ ID NO: 752 - DNA, 12 bp, Mus musculus
  source 1..12: mol_type = genomic DNA
ataatagctatg

SEQ ID NO: 753 - protein, 7 aa, Synthetic construct
QRANLRA

SEQ ID NO: 754 - protein, 7 aa, Synthetic construct
DSGNLRV

SEQ ID NO: 755 - protein, 7 aa, Synthetic construct
RKDNLKN

SEQ ID NO: 756 - protein, 7 aa, Synthetic construct
TTGNLTV

SEQ ID NO: 757 - protein, 7 aa, Synthetic construct
SPADLTR

SEQ ID NO: 758 - protein, 7 aa, Synthetic construct
DKKDLTR

SEQ ID NO: 759 - protein, 7 aa, Synthetic construct
RTDTLRD

SEQ ID NO: 760 - protein, 7 aa, Synthetic construct
THLDLIR

SEQ ID NO: 761 - protein, 7 aa, Synthetic construct
QLAHLRA

SEQ ID NO: 762 - protein, 7 aa, Synthetic construct
ERSHLRE

SEQ ID NO: 763 - protein, 7 aa, Synthetic construct
RSDHLTN

SEQ ID NO: 764 - protein, 7 aa, Synthetic construct
HRTTLTN

SEQ ID NO: 765 - protein, 7 aa, Synthetic construct
QKSSLIA

SEQ ID NO: 766 - protein, 7 aa, Synthetic construct
RRSACRR

SEQ ID NO: 767 - protein, 7 aa, Synthetic construct
RRDELNV

SEQ ID NO: 768 - protein, 7 aa, Synthetic construct
HKNALQN

SEQ ID NO: 769 - protein, 7 aa, Synthetic construct
QSGNLTE

SEQ ID NO: 770 - protein, 7 aa, Synthetic construct
SKKALTE

SEQ ID NO: 771 - protein, 7 aa, Synthetic construct
RADNLTE

SEQ ID NO: 772 - protein, 7 aa, Synthetic construct
TSGNLTE

SEQ ID NO: 773 - protein, 7 aa, Synthetic construct
TSHSLTE

SEQ ID NO: 774 - protein, 7 aa, Synthetic construct
SKKHLAE

SEQ ID NO: 775 - protein, 7 aa, Synthetic construct
RNDTLTE

SEQ ID NO: 776 - protein, 7 aa, Synthetic construct
TKNSLTE

SEQ ID NO: 777 - protein, 7 aa, Synthetic construct
QSGHLTE

SEQ ID NO: 778 - protein, 7 aa, Synthetic construct
HTGHLLE

SEQ ID NO: 779 - protein, 7 aa, Synthetic construct
RSDKLTE

SEQ ID NO: 780 - protein, 7 aa, Synthetic construct
SRRTCRA

SEQ ID NO: 781 - protein, 7 aa, Synthetic construct
QNSTLTE

SEQ ID NO: 782 - protein, 7 aa, Synthetic construct
QRHHLVE

SEQ ID NO: 783 - protein, 7 aa, Synthetic construct
RNDALTE

SEQ ID NO: 784 - protein, 7 aa, Synthetic construct
TTGALTE

SEQ ID NO: 785 - protein, 7 aa, Synthetic construct
QSSNLVR

SEQ ID NO: 786 - protein, 7 aa, Synthetic construct
DPGNLVR

SEQ ID NO: 787 - protein, 7 aa, Synthetic construct
RSDNLVR

SEQ ID NO: 788 - protein, 7 aa, Synthetic construct
TSGNLVR

SEQ ID NO: 789 - protein, 7 aa, Synthetic construct
QSGDLRR

SEQ ID NO: 790 - protein, 7 aa, Synthetic construct
DCRDLAR

SEQ ID NO: 791 - protein, 7 aa, Synthetic construct
RSDDLVR

SEQ ID NO: 792 - protein, 7 aa, Synthetic construct
TSGELVR

SEQ ID NO: 793 - protein, 7 aa, Synthetic construct
QRAHLER

SEQ ID NO: 794 - protein, 7 aa, Synthetic construct
DPGHLVR

SEQ ID NO: 795 - protein, 7 aa, Synthetic construct
RSDKLVR

SEQ ID NO: 796 - protein, 7 aa, Synthetic construct
TSGHLVR

SEQ ID NO: 797 - protein, 7 aa, Synthetic construct
QSSSLVR

SEQ ID NO: 798 - protein, 7 aa, Synthetic construct
DPGALVR

SEQ ID NO: 799 - protein, 7 aa, Synthetic construct
RSDELVR

SEQ ID NO: 800 - protein, 7 aa, Synthetic construct
TSGSLVR

SEQ ID NO: 801 - protein, 7 aa, Homo sapiens
QASNLIS

SEQ ID NO: 802 - protein, 7 aa, Synthetic construct
SRGNLKS

SEQ ID NO: 803 - protein, 7 aa, Synthetic construct
REDNLHT

SEQ ID NO: 804 - protein, 7 aa, Synthetic construct
ARGNLRT

SEQ ID NO: 805 - protein, 7 aa, Synthetic construct
RSDHLTT

SEQ ID NO: 806 - protein, 7 aa, Synthetic construct
RSDERKR

SEQ ID NO: 807 - protein, 7 aa, Synthetic construct
RLRALDR

SEQ ID NO: 808 - protein, 7 aa, Synthetic construct
RLRDIQF

SEQ ID NO: 809 - protein, 7 aa, Synthetic construct
QAGHLAS

SEQ ID NO: 810 - protein, 7 aa, Synthetic construct
APKALGW

SEQ ID NO: 811 - protein, 7 aa, Synthetic construct
RSDHLTT

SEQ ID NO: 812 - protein, 7 aa, Synthetic construct
WRDSLLA

SEQ ID NO: 813 - protein, 7 aa, Synthetic construct
QKWPRDS

SEQ ID NO: 814 - protein, 7 aa, Synthetic construct
DNSYLPR

SEQ ID NO: 815 - protein, 7 aa, Synthetic construct
RKDALRG

SEQ ID NO: 816 - DNA, 21 bp, Synthetic construct
  source 1..21: mol_type = other DNA
cagagagctaatctcagggcc

SEQ ID NO: 817 - DNA, 21 bp, Synthetic construct
  source 1..21: mol_type = other DNA
gattcagggaatctccgggtt

SEQ ID NO: 818 - DNA, 21 bp, Synthetic construct
  source 1..21: mol_type = other DNA
cgaaaagataatctgaagaat

SEQ ID NO: 819 - DNA, 21 bp, Synthetic construct
  source 1..21: mol_type = other DNA
accactggaaacctcacggtg

SEQ ID NO: 820 - DNA, 21 bp, Synthetic construct
  source 1..21: mol_type = other DNA
agtcctgcagatcttacccga

SEQ ID NO: 821 - DNA, 21 bp, Synthetic construct
  source 1..21: mol_type = other DNA
gacaagaaggatctgacacga

SEQ ID NO: 822 - DNA, 21 bp, Synthetic construct
  source 1..21: mol_type = other DNA
aggactgatacgctgcgcgat

SEQ ID NO: 823 - DNA, 21 bp, Synthetic construct
  source 1..21: mol_type = other DNA
acccacctggacctcatcaga

SEQ ID NO: 824 - DNA, 21 bp, Synthetic construct
  source 1..21: mol_type = other DNA
caactcgctcatctgcgagca

SEQ ID NO: 825 - DNA, 21 bp, Synthetic construct
  source 1..21: mol_type = other DNA
gaacgaagccacctgcgcgaa

SEQ ID NO: 826 - DNA, 21 bp, Synthetic construct
  source 1..21: mol_type = other DNA
cgcagcgaccatttgactaac

SEQ ID NO: 827 - DNA, 21 bp, Synthetic construct
  source 1..21: mol_type = other DNA
caccgaacgaccttgactaac

SEQ ID NO: 828 - DNA, 21 bp, Synthetic construct
  source 1..21: mol_type = other DNA
cagaaatcttctttgatagct

SEQ ID NO: 829 - DNA, 21 bp, Synthetic construct
  source 1..21: mol_type = other DNA
cggagatcagcctgtcgacgc

SEQ ID NO: 830 - DNA, 21 bp, Synthetic construct
  source 1..21: mol_type = other DNA
aggcgggacgaactgaacgtg

SEQ ID NO: 831 - DNA, 21 bp, Synthetic construct
  source 1..21: mol_type = other DNA
cacaaaaatgccttgcaaaac

SEQ ID NO: 832 - DNA, 21 bp, Synthetic construct
  source 1..21: mol_type = other DNA
caatctggcaatcttacagag

SEQ ID NO: 833 - DNA, 21 bp, Synthetic construct
  source 1..21: mol_type = other DNA
tctaaaaaggcgctgacggag

SEQ ID NO: 834 - DNA, 21 bp, Synthetic construct
  source 1..21: mol_type = other DNA
cgggcggataatctcactgag

SEQ ID NO: 835 - DNA, 21 bp, Synthetic construct
  source 1..21: mol_type = other DNA
acgagtggaaatcttacggaa

SEQ ID NO: 836 - DNA, 21 bp, Synthetic construct
  source 1..21: mol_type = other DNA
acgtcccacagtttgaccgaa

SEQ ID NO: 837 - DNA, 21 bp, Synthetic construct
  source 1..21: mol_type = other DNA
agcaagaaacaccttgcagaa

SEQ ID NO: 838 - DNA, 21 bp, Synthetic construct
  source 1..21: mol_type = other DNA
aggaatgatactcttaccgag

SEQ ID NO: 839 - DNA, 21 bp, Synthetic construct
  source 1..21: mol_type = other DNA
acaaagaacagcctcaccgag

SEQ ID NO: 840 - DNA, 21 bp, Synthetic construct
  source 1..21: mol_type = other DNA
cagtcagggcatctcacggag

SEQ ID NO: 841 - DNA, 21 bp, Synthetic construct
  source 1..21: mol_type = other DNA
cacacaggccatttgttggag

SEQ ID NO: 842 - DNA, 21 bp, Synthetic construct
  source 1..21: mol_type = other DNA
cggagtgataaactcaccgaa

SEQ ID NO: 843 - DNA, 21 bp, Synthetic construct
  source 1..21: mol_type = other DNA
tcacgacgcacctgtagagcg

SEQ ID NO: 844 - DNA, 21 bp, Synthetic construct
  source 1..21: mol_type = other DNA
cagaattcaactctcaccgaa

SEQ ID NO: 845 - DNA, 21 bp, Synthetic construct
  source 1..21: mol_type = other DNA
cagcgacaccatttggtcgag

SEQ ID NO: 846 - DNA, 21 bp, Synthetic construct
  source 1..21: mol_type = other DNA
cggaacgatgcacttaccgag

SEQ ID NO: 847 - DNA, 21 bp, Synthetic construct
  source 1..21: mol_type = other DNA
actacaggggctctcactgaa

SEQ ID NO: 848 - DNA, 21 bp, Synthetic construct
  source 1..21: mol_type = other DNA
cagagtagtaacctggtgagg

SEQ ID NO: 849 - DNA, 21 bp, Synthetic construct
  source 1..21: mol_type = other DNA
gatcccgggaacctcgttaga

SEQ ID NO: 850 - DNA, 21 bp, Synthetic construct
  source 1..21: mol_type = other DNA
cgctctgataacctggtcaga

SEQ ID NO: 851 - DNA, 21 bp, Synthetic construct
  source 1..21: mol_type = other DNA
actagcgggaacctcgtccgg

SEQ ID NO: 852 - DNA, 21 bp, Synthetic construct
  source 1..21: mol_type = other DNA
caaagcggggacttgagaagg

SEQ ID NO: 853 - DNA, 21 bp, Synthetic construct
  source 1..21: mol_type = other DNA
gattgccgagatcttgctcgg

SEQ ID NO: 854 - DNA, 21 bp, Synthetic construct
  source 1..21: mol_type = other DNA
cgctcagatgatctggttcgc

SEQ ID NO: 855 - DNA, 21 bp, Synthetic construct
  source 1..21: mol_type = other DNA
acgtctggggagttggttagg

SEQ ID NO: 856 - DNA, 21 bp, Synthetic construct
  source 1..21: mol_type = other DNA
caaagagcccatctggaaagg

SEQ ID NO: 857 - DNA, 21 bp, Synthetic construct
  source 1..21: mol_type = other DNA
gatcccggacacttggttcga

SEQ ID NO: 858 - DNA, 21 bp, Synthetic construct
  source 1..21: mol_type = other DNA
cgcagcgacaaactcgttaga

SEQ ID NO: 859 - DNA, 21 bp, Synthetic construct
  source 1..21: mol_type = other DNA
acttcaggccatcttgtaaga

SEQ ID NO: 860 - DNA, 21 bp, Synthetic construct
  source 1..21: mol_type = other DNA
caatcttcctcacttgtgagg

SEQ ID NO: 861 - DNA, 21 bp, Synthetic construct
  source 1..21: mol_type = other DNA
gacccaggggctttggttcgg

SEQ ID NO: 862 - DNA, 21 bp, Synthetic construct
  source 1..21: mol_type = other DNA
cggtcagatgagctggtacgc

SEQ ID NO: 863 - DNA, 21 bp, Synthetic construct
  source 1..21: mol_type = other DNA
acaagcggctctctcgttaga

SEQ ID NO: 864 - DNA, 21 bp, Synthetic construct
  source 1..21: mol_type = other DNA
caagcctctaacttgattagc

SEQ ID NO: 865 - DNA, 21 bp, Synthetic construct
  source 1..21: mol_type = other DNA
agcaggggtaacttgaaatcc

SEQ ID NO: 866 - DNA, 21 bp, Synthetic construct
  source 1..21: mol_type = other DNA
cgggaagacaaccttcatacg

SEQ ID NO: 867 - DNA, 21 bp, Synthetic construct
  source 1..21: mol_type = other DNA
gcacgcgggaacttgcggact

SEQ ID NO: 868 - DNA, 21 bp, Synthetic construct
  source 1..21: mol_type = other DNA
cgaagtgatcacttgacaacc

SEQ ID NO: 869 - DNA, 21 bp, Synthetic construct
  source 1..21: mol_type = other DNA
cggtcagacgagagaaagcga

SEQ ID NO: 870 - DNA, 21 bp, Synthetic construct
  source 1..21: mol_type = other DNA
cgcttgcgggcgctcgaccga

SEQ ID NO: 871 - DNA, 21 bp, Synthetic construct
  source 1..21: mol_type = other DNA
agactcagggatatacaattt

SEQ ID NO: 872 - DNA, 21 bp, Synthetic construct
  source 1..21: mol_type = other DNA
caagcgggccacctcgccagc

SEQ ID NO: 873 - DNA, 21 bp, Synthetic construct
  source 1..21: mol_type = other DNA
gccccaaaagcactgggctgg

SEQ ID NO: 874 - DNA, 21 bp, Synthetic construct
  source 1..21: mol_type = other DNA
cggagcgaccatctcactact

SEQ ID NO: 875 - DNA, 21 bp, Synthetic construct
  source 1..21: mol_type = other DNA
tggcgcgactcccttctcgcg

SEQ ID NO: 876 - DNA, 21 bp, Synthetic construct
  source 1..21: mol_type = other DNA
cagaagtggcccagggattca

SEQ ID NO: 877 - DNA, 21 bp, Synthetic construct
  source 1..21: mol_type = other DNA
gacaattcttacttgcccagg

SEQ ID NO: 878 - DNA, 21 bp, Synthetic construct
  source 1..21: mol_type = other DNA
aggaaagatgcgcttagaggg

SEQ ID NO: 879 - protein, 7 aa, Synthetic construct
KSSNLRR

SEQ ID NO: 880 - protein, 7 aa, Synthetic construct
QKSNLIR

SEQ ID NO: 881 - protein, 7 aa, Synthetic construct
QSGSLTR

SEQ ID NO: 882 - protein, 7 aa, Synthetic construct
QSNTLKQ

SEQ ID NO: 883 - protein, 7 aa, Synthetic construct
QSSHLNV

SEQ ID NO: 884 - protein, 7 aa, Synthetic construct
QSSSLIR

SEQ ID NO: 885 - protein, 7 aa, Synthetic construct
QSTHLTQ

SEQ ID NO: 886 - protein, 7 aa, Synthetic construct
RSDALSQ

SEQ ID NO: 887 - protein, 7 aa, Synthetic construct
RSDNLST

SEQ ID NO: 888 - protein, 7 aa, Synthetic construct
RSDNLTQ

SEQ ID NO: 889 - protein, 7 aa, Synthetic construct
TSGSLSR

SEQ ID NO: 890 - protein, 7 aa, Synthetic construct
VKDYLTK

SEQ ID NO: 891 - protein, 7 aa, Synthetic construct
WPSNLTR

SEQ ID NO: 892 - DNA, 12 bp, Synthetic construct
  source 1..12: mol_type = other DNA
gagtaggaggca

SEQ ID NO: 893 - DNA, 12 bp, Synthetic construct
  source 1..12: mol_type = other DNA
gtggaggccacg

SEQ ID NO: 894 - DNA, 12 bp, Synthetic construct
  source 1..12: mol_type = other DNA
gagtaggagtgt

SEQ ID NO: 895 - DNA, 12 bp, Synthetic construct
  source 1..12: mol_type = other DNA
gtggaggcctag

SEQ ID NO: 896 - DNA, 12 bp, Synthetic construct
  source 1..12: mol_type = other DNA
gaggtcttggta

SEQ ID NO: 897 - DNA, 12 bp, Synthetic construct
  source 1..12: mol_type = other DNA
gttgaggtcttg

SEQ ID NO: 898 - DNA, 12 bp, Synthetic construct
  source 1..12: mol_type = other DNA
cctgaggcatgg

SEQ ID NO: 899 - DNA, 12 bp, Synthetic construct
  source 1..12: mol_type = other DNA
gaggctccaagc

SEQ ID NO: 900 - DNA, 12 bp, Synthetic construct
  source 1..12: mol_type = other DNA
gcaggagaagat

SEQ ID NO: 901 - DNA, 15 bp, Synthetic construct
  source 1..15: mol_type = other DNA
gagtaggaggcaaat

SEQ ID NO: 902 - DNA, 15 bp, Synthetic construct
  source 1..15: mol_type = other DNA
gtggaggccacggct

SEQ ID NO: 903 - DNA, 15 bp, Synthetic construct
  source 1..15: mol_type = other DNA
gagtaggagtgtcag

SEQ ID NO: 904 - DNA, 15 bp, Synthetic construct
  source 1..15: mol_type = other DNA
gtggaggcctagaca

SEQ ID NO: 905 - DNA, 15 bp, Synthetic construct
  source 1..15: mol_type = other DNA
gaggtcttggtaagt

SEQ ID NO: 906 - DNA, 15 bp, Synthetic construct
  source 1..15: mol_type = other DNA
gttgaggtcttggta

SEQ ID NO: 907 - DNA, 15 bp, Synthetic construct
  source 1..15: mol_type = other DNA
tatcctgaggcatgg

SEQ ID NO: 908 - DNA, 15 bp, Synthetic construct
  source 1..15: mol_type = other DNA
gaggctccaagcaaa

SEQ ID NO: 909 - DNA, 15 bp, Synthetic construct
  source 1..15: mol_type = other DNA
agggcaggagaagat

SEQ ID NO: 910 - DNA, 18 bp, Synthetic construct
  source 1..18: mol_type = other DNA
gagtaggaggcaaatctc

SEQ ID NO: 911 - DNA, 18 bp, Synthetic construct
  source 1..18: mol_type = other DNA
gtggaggccacggctctg

SEQ ID NO: 912 - DNA, 18 bp, Synthetic construct
  source 1..18: mol_type = other DNA
gagtaggagtgtcaggtt

SEQ ID NO: 913 - DNA, 18 bp, Synthetic construct
  source 1..18: mol_type = other DNA
gtggaggcctagacagcg

SEQ ID NO: 914 - DNA, 18 bp, Synthetic construct
  source 1..18: mol_type = other DNA
gaggtcttggtaagtgtt

SEQ ID NO: 915 - DNA, 18 bp, Synthetic construct
  source 1..18: mol_type = other DNA
gttgaggtcttggtaagt

SEQ ID NO: 916 - DNA, 18 bp, Synthetic construct
  source 1..18: mol_type = other DNA
gagtatcctgaggcatgg

SEQ ID NO: 917 - DNA, 18 bp, Synthetic construct
  source 1..18: mol_type = other DNA
gaggctccaagcaaaatt

SEQ ID NO: 918 - DNA, 18 bp, Synthetic construct
  source 1..18: mol_type = other DNA
ggaagggcaggagaagat

SEQ ID NO: 919 - DNA, 18 bp, Synthetic construct
  source 1..18: mol_type = other DNA
taagcaatagatggctct

SEQ ID NO: 920 - protein, 30 aa, Synthetic construct
AIPVKRGATGETKVFIGNSNSPKSPTKGGC

SEQ ID NO: 921 - protein, 29 aa, Synthetic construct
IPVKRGATGETKVFIGNSNSPKSPTKGGC

SEQ ID NO: 922 - protein, 28 aa, Synthetic construct
PVKRGATGETKVFIGNSNSPKSPTKGGC

SEQ ID NO: 923 - protein, 27 aa, Synthetic construct
VKRGATGETKVFIGNSNSPKSPTKGGC

SEQ ID NO: 924 - protein, 26 aa, Synthetic construct
KRGATGETKVFIGNSNSPKSPTKGGC

SEQ ID NO: 925 - protein, 25 aa, Synthetic construct
RGATGETKVFIGNSNSPKSPTKGGC

SEQ ID NO: 926 - protein, 24 aa, Synthetic construct
GATGETKVFIGNSNSPKSPTKGGC

SEQ ID NO: 927 - protein, 23 aa, Synthetic construct
ATGETKVFIGNSNSPKSPTKGGC

SEQ ID NO: 928 - protein, 22 aa, Synthetic construct
TGETKVFIGNSNSPKSPTKGGC

SEQ ID NO: 929 - protein, 21 aa, Synthetic construct
GETKVFIGNSNSPKSPTKGGC

SEQ ID NO: 930 - protein, 20 aa, Synthetic construct
ETKVFIGNSNSPKSPTKGGC

SEQ ID NO: 931 - protein, 19 aa, Synthetic construct
TKVFIGNSNSPKSPTKGGC

SEQ ID NO: 932 - protein, 18 aa, Synthetic construct
KVFIGNSNSPKSPTKGGC

SEQ ID NO: 933 - protein, 17 aa, Synthetic construct
VFIGNSNSPKSPTKGGC

SEQ ID NO: 934 - protein, 16 aa, Synthetic construct
FIGNSNSPKSPTKGGC

SEQ ID NO: 935 - protein, 15 aa, Synthetic construct
IGNSNSPKSPTKGGC

SEQ ID NO: 936 - protein, 29 aa, Synthetic construct
AIPVKRGATGETKVFIGNSNSPKSPTKGG

SEQ ID NO: 937 - protein, 28 aa, Synthetic construct
AIPVKRGATGETKVFIGNSNSPKSPTKG

SEQ ID NO: 938 - protein, 27 aa, Synthetic construct
AIPVKRGATGETKVFIGNSNSPKSPTK

SEQ ID NO: 939 - protein, 26 aa, Synthetic construct
AIPVKRGATGETKVFIGNSNSPKSPT

SEQ ID NO: 940 - protein, 25 aa, Synthetic construct
AIPVKRGATGETKVFIGNSNSPKSP

SEQ ID NO: 941 - protein, 24 aa, Synthetic construct
AIPVKRGATGETKVFIGNSNSPKS

SEQ ID NO: 942 - protein, 23 aa, Synthetic construct
AIPVKRGATGETKVFIGNSNSPK

SEQ ID NO: 943 - protein, 22 aa, Synthetic construct
AIPVKRGATGETKVFIGNSNSP

SEQ ID NO: 944 - protein, 21 aa, Synthetic construct
AIPVKRGATGETKVFIGNSNS

SEQ ID NO: 945 - protein, 108 aa, Synthetic construct
GSYALGPYQISAPQLPAYNGQTVGTFYYVNDAGGLESKVFSSGGPTPYPNYANAGHVEGQSALFMRDNGISEGLVFHNNPEGTCGFCVNMIETLLPENAKMTVVPPKG

SEQ ID NO: 946 - protein, 107 aa, Synthetic construct
GSYALGPYQISAPQLPAYNGQTVGTFYYVNDAGGLESKVFSSGGPTPYPNYANAGHVEGQSALFMRDNGISEGLVFHNNPEGTCGFCVNMIETLLPENAKMTVVPPK

SEQ ID NO: 947 - protein, 106 aa, Synthetic construct
GSYALGPYQISAPQLPAYNGQTVGTFYYVNDAGGLESKVFSSGGPTPYPNYANAGHVEGQSALFMRDNGISEGLVFHNNPEGTCGFCVNMIETLLPENAKMTVVPP

SEQ ID NO: 948 - protein, 105 aa, Synthetic construct
GSYALGPYQISAPQLPAYNGQTVGTFYYVNDAGGLESKVFSSGGPTPYPNYANAGHVEGQSALFMRDNGISEGLVFHNNPEGTCGFCVNMIETLLPENAKMTVVP

SEQ ID NO: 949 - protein, 104 aa, Synthetic construct
GSYALGPYQISAPQLPAYNGQTVGTFYYVNDAGGLESKVFSSGGPTPYPNYANAGHVEGQSALFMRDNGISEGLVFHNNPEGTCGFCVNMIETLLPENAKMTVV

SEQ ID NO: 950 - protein, 103 aa, Synthetic construct
GSYALGPYQISAPQLPAYNGQTVGTFYYVNDAGGLESKVFSSGGPTPYPNYANAGHVEGQSALFMRDNGISEGLVFHNNPEGTCGFCVNMIETLLPENAKMTV

SEQ ID NO: 951 - protein, 102 aa, Synthetic construct
GSYALGPYQISAPQLPAYNGQTVGTFYYVNDAGGLESKVFSSGGPTPYPNYANAGHVEGQSALFMRDNGISEGLVFHNNPEGTCGFCVNMIETLLPENAKMT

SEQ ID NO: 952 - protein, 109 aa, Synthetic construct
GSYALGPYQISAPQLPAYNGQTVGTFYYVNDAGGLESKVFSSGGPTPYPNYANAGHVEGQSALFMRDNGISEGLVFHNNPEGTCGFCVNMIETLLPENAKMTVVPPKGA

SEQ ID NO: 953 - protein, 110 aa, Synthetic construct
GSYALGPYQISAPQLPAYNGQTVGTFYYVNDAGGLESKVFSSGGPTPYPNYANAGHVEGQSALFMRDNGISEGLVFHNNPEGTCGFCVNMIETLLPENAKMTVVPPKGAI

SEQ ID NO: 954 - protein, 111 aa, Synthetic construct
GSYALGPYQISAPQLPAYNGQTVGTFYYVNDAGGLESKVFSSGGPTPYPNYANAGHVEGQSALFMRDNGISEGLVFHNNPEGTCGFCVNMIETLLPENAKMTVVPPKGAIP

SEQ ID NO: 955 - protein, 112 aa, Synthetic construct
GSYALGPYQISAPQLPAYNGQTVGTFYYVNDAGGLESKVFSSGGPTPYPNYANAGHVEGQSALFMRDNGISEGLVFHNNPEGTCGFCVNMIETLLPENAKMTVVPPKGAIPV

SEQ ID NO: 956 - protein, 113 aa, Synthetic construct
GSYALGPYQISAPQLPAYNGQTVGTFYYVNDAGGLESKVFSSGGPTPYPNYANAGHVEGQSALFMRDNGISEGLVFHNNPEGTCGFCVNMIETLLPENAKMTVVPPKGAIPVK

SEQ ID NO: 957 - protein, 114 aa, Synthetic construct
GSYALGPYQISAPQLPAYNGQTVGTFYYVNDAGGLESKVFSSGGPTPYPNYANAGHVEGQSALFMRDNGISEGLVFHNNPEGTCGFCVNMIETLLPENAKMTVVPPKGAIPVKR

SEQ ID NO: 958 - protein, 115 aa, Synthetic construct
GSYALGPYQISAPQLPAYNGQTVGTFYYVNDAGGLESKVFSSGGPTPYPNYANAGHVEGQSALFMRDNGISEGLVFHNNPEGTCGFCVNMIETLLPENAKMTVVPPKGAIPVKRG

SEQ ID NO: 959 - protein, 116 aa, Synthetic construct
GSYALGPYQISAPQLPAYNGQTVGTFYYVNDAGGLESKVFSSGGPTPYPNYANAGHVEGQSALFMRDNGISEGLVFHNNPEGTCGFCVNMIETLLPENAKMTVVPPKGAIPVKRGA

SEQ ID NO: 960 - protein, 117 aa, Synthetic construct
GSYALGPYQISAPQLPAYNGQTVGTFYYVNDAGGLESKVFSSGGPTPYPNYANAGHVEGQSALFMRDNGISEGLVFHNNPEGTCGFCVNMIETLLPENAKMTVVPPKGAIPVKRGAT

SEQ ID NO: 961 - protein, 118 aa, Synthetic construct
GSYALGPYQISAPQLPAYNGQTVGTFYYVNDAGGLESKVFSSGGPTPYPNYANAGHVEGQSALFMRDNGISEGLVFHNNPEGTCGFCVNMIETLLPENAKMTVVPPKGAIPVKRGATG

SEQ ID NO: 962 - protein, 119 aa, Synthetic construct
GSYALGPYQISAPQLPAYNGQTVGTFYYVNDAGGLESKVFSSGGPTPYPNYANAGHVEGQSALFMRDNGISEGLVFHNNPEGTCGFCVNMIETLLPENAKMTVVPPKGAIPVKRGATGE

SEQ ID NO: 963 - protein, 120 aa, Synthetic construct
GSYALGPYQISAPQLPAYNGQTVGTFYYVNDAGGLESKVFSSGGPTPYPNYANAGHVEGQSALFMRDNGISEGLVFHNNPEGTCGFCVNMIETLLPENAKMTVVPPKGAIPVKRGATGET

SEQ ID NO: 964 - protein, 121 aa, Synthetic construct
GSYALGPYQISAPQLPAYNGQTVGTFYYVNDAGGLESKVFSSGGPTPYPNYANAGHVEGQSALFMRDNGISEGLVFHNNPEGTCGFCVNMIETLLPENAKMTVVPPKGAIPVKRGATGETK

SEQ ID NO: 965 - protein, 122 aa, Synthetic construct
GSYALGPYQISAPQLPAYNGQTVGTFYYVNDAGGLESKVFSSGGPTPYPNYANAGHVEGQSALFMRDNGISEGLVFHNNPEGTCGFCVNMIETLLPENAKMTVVPPKGAIPVKRGATGETKV

SEQ ID NO: 966 - protein, 123 aa, Synthetic construct
GSYALGPYQISAPQLPAYNGQTVGTFYYVNDAGGLESKVFSSGGPTPYPNYANAGHVEGQSALFMRDNGISEGLVFHNNPEGTCGFCVNMIETLLPENAKMTVVPPKGAIPVKRGATGETKVF

SEQ ID NO: 967 - protein, 30 aa, Synthetic construct; identical to SEQ ID NO: 920

SEQ ID NO: 968 - protein, 30 aa, Synthetic construct
AAPVKRGATGETKVFIGNSNSPKSPTKGGC

SEQ ID NO: 969 - protein, 30 aa, Synthetic construct
AIAVKRGATGETKVFIGNSNSPKSPTKGGC

SEQ ID NO: 970 - protein, 30 aa, Synthetic construct
AIPAKRGATGETKVFIGNSNSPKSPTKGGC

SEQ ID NO: 971 - protein, 30 aa, Synthetic construct
AIPVARGATGETKVFIGNSNSPKSPTKGGC

SEQ ID NO: 972 - protein, 30 aa, Synthetic construct
AIPVKAGATGETKVFIGNSNSPKSPTKGGC

SEQ ID NO: 973 - protein, 30 aa, Synthetic construct
AIPVKRAATGETKVFIGNSNSPKSPTKGGC

SEQ ID NO: 974 - protein, 30 aa, Synthetic construct
AIPVKRGAAGETKVFIGNSNSPKSPTKGGC

SEQ ID NO: 975 - protein, 30 aa, Synthetic construct
AIPVKRGATAETKVFIGNSNSPKSPTKGGC

SEQ ID NO: 976 - protein, 30 aa, Synthetic construct
AIPVKRGATGATKVFIGNSNSPKSPTKGGC

SEQ ID NO: 977 - protein, 30 aa, Synthetic construct
AIPVKRGATGEAKVFIGNSNSPKSPTKGGC

SEQ ID NO: 978 - protein, 30 aa, Synthetic construct
AIPVKRGATGETAVFIGNSNSPKSPTKGGC

SEQ ID NO: 979 - protein, 30 aa, Synthetic construct
AIPVKRGATGETKAFIGNSNSPKSPTKGGC

SEQ ID NO: 980 - protein, 30 aa, Synthetic construct
AIPVKRGATGETKVAIGNSNSPKSPTKGGC

SEQ ID NO: 981 - protein, 30 aa, Synthetic construct; identical to SEQ ID NO: 153

SEQ ID NO: 982 - protein, 30 aa, Synthetic construct
AIPVKRGATGETKVFIANSNSPKSPTKGGC

SEQ ID NO: 983 - protein, 30 aa, Synthetic construct
AIPVKRGATGETKVFIGASNSPKSPTKGGC

SEQ ID NO: 984 - protein, 30 aa, Synthetic construct
AIPVKRGATGETKVFIGNANSPKSPTKGGC

SEQ ID NO: 985 - protein, 30 aa, Synthetic construct
AIPVKRGATGETKVFIGNSASPKSPTKGGC

SEQ ID NO: 986 - protein, 30 aa, Synthetic construct
AIPVKRGATGETKVFIGNSNAPKSPTKGGC

SEQ ID NO: 987 - protein, 30 aa, Synthetic construct
AIPVKRGATGETKVFIGNSNSAKSPTKGGC

SEQ ID NO: 988 - protein, 30 aa, Synthetic construct
AIPVKRGATGETKVFIGNSNSPASPTKGGC

SEQ ID NO: 989 - protein, 30 aa, Synthetic construct
AIPVKRGATGETKVFIGNSNSPKAPTKGGC

SEQ ID NO: 990 - protein, 30 aa, Synthetic construct
AIPVKRGATGETKVFIGNSNSPKSATKGGC

SEQ ID NO: 991 - protein, 30 aa, Synthetic construct
AIPVKRGATGETKVFIGNSNSPKSPAKGGC

SEQ ID NO: 992 - protein, 30 aa, Synthetic construct
AIPVKRGATGETKVFIGNSNSPKSPTAGGC

SEQ ID NO: 993 - protein, 30 aa, Synthetic construct
AIPVKRGATGETKVFIGNSNSPKSPTKAGC

SEQ ID NO: 994 - protein, 30 aa, Synthetic construct
AIPVKRGATGETKVFIGNSNSPKSPTKGAC

SEQ ID NO: 995 - protein, 30 aa, Synthetic construct
AIPVKRGATGETKVFIGNSNSPKSPTKGGA

SEQ ID NO: 996 - protein, 30 aa, Synthetic construct; identical to SEQ ID NO: 920

SEQ ID NO: 997 - protein, 30 aa, Synthetic construct
KIPVKRGATGETKVFIGNSNSPKSPTKGGC

SEQ ID NO: 998 - protein, 30 aa, Synthetic construct
AKPVKRGATGETKVFIGNSNSPKSPTKGGC

SEQ ID NO: 999 - protein, 30 aa, Synthetic construct
AIKVKRGATGETKVFIGNSNSPKSPTKGGC

SEQ ID NO: 1000 - protein, 30 aa, Synthetic construct
AIPKKRGATGETKVFIGNSNSPKSPTKGGC

SEQ ID NO: 1001 - protein, 30 aa, Synthetic construct
AIPVKKGATGETKVFIGNSNSPKSPTKGGC

SEQ ID NO: 1002 - protein, 30 aa, Synthetic construct
AIPVKRKATGETKVFIGNSNSPKSPTKGGC

SEQ ID NO: 1003 - protein, 30 aa, Synthetic construct
AIPVKRGKTGETKVFIGNSNSPKSPTKGGC

SEQ ID NO: 1004 - protein, 30 aa, Synthetic construct
AIPVKRGAKGETKVFIGNSNSPKSPTKGGC

SEQ ID NO: 1005 - protein, 30 aa, Synthetic construct
AIPVKRGATKETKVFIGNSNSPKSPTKGGC

SEQ ID NO: 1006 - protein, 30 aa, Synthetic construct
AIPVKRGATGKTKVFIGNSNSPKSPTKGGC

SEQ ID NO: 1007 - protein, 30 aa, Synthetic construct
AIPVKRGATGEKKVFIGNSNSPKSPTKGGC

SEQ ID NO: 1008 - protein, 30 aa, Synthetic construct
AIPVKRGATGETKKFIGNSNSPKSPTKGGC

SEQ ID NO: 1009 - protein, 30 aa, Synthetic construct
AIPVKRGATGETKVKIGNSNSPKSPTKGGC

SEQ ID NO: 1010 - protein, 30 aa, Synthetic construct; identical to SEQ ID NO: 181

SEQ ID NO: 1011 - protein, 30 aa, Synthetic construct
AIPVKRGATGETKVFIKNSNSPKSPTKGGC

SEQ ID NO: 1012 - protein, 30 aa, Synthetic construct
AIPVKRGATGETKVFIGKSNSPKSPTKGGC

SEQ ID NO: 1013 - protein, 30 aa, Synthetic construct
AIPVKRGATGETKVFIGNKNSPKSPTKGGC

SEQ ID NO: 1014 - protein, 30 aa, Synthetic construct
AIPVKRGATGETKVFIGNSKSPKSPTKGGC

SEQ ID NO: 1015 - protein, 30 aa, Synthetic construct
AIPVKRGATGETKVFIGNSNKPKSPTKGGC

SEQ ID NO: 1016 - protein, 30 aa, Synthetic construct
AIPVKRGATGETKVFIGNSNSKKSPTKGGC

SEQ ID NO: 1017 - protein, 30 aa, Synthetic construct
AIPVKRGATGETKVFIGNSNSPKKPTKGGC

SEQ ID NO: 1018 - protein, 30 aa, Synthetic construct
AIPVKRGATGETKVFIGNSNSPKSKTKGGC

SEQ ID NO: 1019 - protein, 30 aa, Synthetic construct
AIPVKRGATGETKVFIGNSNSPKSPKKGGC

SEQ ID NO: 1020 - protein, 30 aa, Synthetic construct
AIPVKRGATGETKVFIGNSNSPKSPTKKGC

SEQ ID NO: 1021 - protein, 30 aa, Synthetic construct
AIPVKRGATGETKVFIGNSNSPKSPTKGKC

SEQ ID NO: 1022 - protein, 30 aa, Synthetic construct
AIPVKRGATGETKVFIGNSNSPKSPTKGGK

SEQ ID NO: 1023 - protein, 30 aa, Synthetic construct; identical to SEQ ID NO: 920

SEQ ID NO: 1024 - protein, 30 aa, Synthetic construct
DIPVKRGATGETKVFIGNSNSPKSPTKGGC

SEQ ID NO: 1025 - protein, 30 aa, Synthetic construct
ADPVKRGATGETKVFIGNSNSPKSPTKGGC

SEQ ID NO: 1026 - protein, 30 aa, Synthetic construct
AIDVKRGATGETKVFIGNSNSPKSPTKGGC

SEQ ID NO: 1027 - protein, 30 aa, Synthetic construct
AIPDKRGATGETKVFIGNSNSPKSPTKGGC

SEQ ID NO: 1028 - protein, 30 aa, Synthetic construct
AIPVDRGATGETKVFIGNSNSPKSPTKGGC

SEQ ID NO: 1029 - protein, 30 aa, Synthetic construct
AIPVKDGATGETKVFIGNSNSPKSPTKGGC

SEQ ID NO: 1030 - protein, 30 aa, Synthetic construct
AIPVKRDATGETKVFIGNSNSPKSPTKGGC

SEQ ID NO: 1031 - protein, 30 aa, Synthetic construct
AIPVKRGDTGETKVFIGNSNSPKSPTKGGC

SEQ ID NO: 1032 - protein, 30 aa, Synthetic construct
AIPVKRGADGETKVFIGNSNSPKSPTKGGC

SEQ ID NO: 1033 - protein, 30 aa, Synthetic construct
AIPVKRGATDETKVFIGNSNSPKSPTKGGC

SEQ ID NO: 1034 - protein, 30 aa, Synthetic construct
AIPVKRGATGDTKVFIGNSNSPKSPTKGGC

SEQ ID NO: 1035 - protein, 30 aa, Synthetic construct
AIPVKRGATGEDKVFIGNSNSPKSPTKGGC

SEQ ID NO: 1036 - protein, 30 aa, Synthetic construct
AIPVKRGATGETDVFIGNSNSPKSPTKGGC

SEQ ID NO: 1037 - protein, 30 aa, Synthetic construct
AIPVKRGATGETKDFIGNSNSPKSPTKGGC

SEQ ID NO: 1038 - protein, 30 aa, Synthetic construct
AIPVKRGATGETKVDIGNSNSPKSPTKGGC

SEQ ID NO: 1039 - protein, 30 aa, Synthetic construct; identical to SEQ ID NO: 209

SEQ ID NO: 1040 - protein, 30 aa, Synthetic construct
AIPVKRGATGETKVFIDNSNSPKSPTKGGC

SEQ ID NO: 1041 - protein, 30 aa, Synthetic construct
AIPVKRGATGETKVFIGDSNSPKSPTKGGC

SEQ ID NO: 1042 - protein, 30 aa, Synthetic construct
AIPVKRGATGETKVFIGNDNSPKSPTKGGC

SEQ ID NO: 1043 - protein, 30 aa, Synthetic construct
AIPVKRGATGETKVFIGNSDSPKSPTKGGC

SEQ ID NO: 1044 - protein, 30 aa, Synthetic construct
AIPVKRGATGETKVFIGNSNDPKSPTKGGC

SEQ ID NO: 1045 - protein, 30 aa, Synthetic construct
AIPVKRGATGETKVFIGNSNSDKSPTKGGC

SEQ ID NO: 1046 - protein, 30 aa, Synthetic construct
AIPVKRGATGETKVFIGNSNSPDSPTKGGC

SEQ ID NO: 1047 - protein, 30 aa, Synthetic construct
AIPVKRGATGETKVFIGNSNSPKDPTKGGC

SEQ ID NO: 1048 - protein, 30 aa, Synthetic construct
AIPVKRGATGETKVFIGNSNSPKSDTKGGC

SEQ ID NO: 1049 - protein, 30 aa, Synthetic construct
AIPVKRGATGETKVFIGNSNSPKSPDKGGC

SEQ ID NO: 1050 - protein, 30 aa, Synthetic construct
AIPVKRGATGETKVFIGNSNSPKSPTDGGC

SEQ ID NO: 1051 - protein, 30 aa, Synthetic construct
AIPVKRGATGETKVFIGNSNSPKSPTKDGC

SEQ ID NO: 1052 - protein, 30 aa, Synthetic construct
AIPVKRGATGETKVFIGNSNSPKSPTKGDC

SEQ ID NO: 1053 - protein, 30 aa, Synthetic construct
AIPVKRGATGETKVFIGNSNSPKSPTKGGD

SEQ ID NO: 1054 - protein, 30 aa, Synthetic construct; identical to SEQ ID NO: 920

SEQ ID NO: 1055 - protein, 30 aa, Synthetic construct
EIPVKRGATGETKVFIGNSNSPKSPTKGGC

SEQ ID NO: 1056 - protein, 30 aa, Synthetic construct
AEPVKRGATGETKVFIGNSNSPKSPTKGGC

SEQ ID NO: 1057 - protein, 30 aa, Synthetic construct
AIEVKRGATGETKVFIGNSNSPKSPTKGGC

SEQ ID NO: 1058 - protein, 30 aa, Synthetic construct
AIPEKRGATGETKVFIGNSNSPKSPTKGGC

SEQ ID NO: 1059 - protein, 30 aa, Synthetic construct
AIPVERGATGETKVFIGNSNSPKSPTKGGC

SEQ ID NO: 1060 - protein, 30 aa, Synthetic construct
AIPVKEGATGETKVFIGNSNSPKSPTKGGC

SEQ ID NO: 1061 - protein, 30 aa, Synthetic construct
AIPVKREATGETKVFIGNSNSPKSPTKGGC

SEQ ID NO: 1062 - protein, 30 aa, Synthetic construct
AIPVKRGETGETKVFIGNSNSPKSPTKGGC

SEQ ID NO: 1063 - protein, 30 aa, Synthetic construct
AIPVKRGAEGETKVFIGNSNSPKSPTKGGC

SEQ ID NO: 1064 - protein, 30 aa, Synthetic construct
AIPVKRGATEETKVFIGNSNSPKSPTKGGC

SEQ ID NO: 1065 - protein, 30 aa, Synthetic construct
AIPVKRGATGEEKVFIGNSNSPKSPTKGGC

SEQ ID NO: 1066 - protein, 30 aa, Synthetic construct
AIPVKRGATGETEVFIGNSNSPKSPTKGGC

SEQ ID NO: 1067 - protein, 30 aa, Synthetic construct
AIPVKRGATGETKEFIGNSNSPKSPTKGGC

SEQ ID NO: 1068 - protein, 30 aa, Synthetic construct
AIPVKRGATGETKVEIGNSNSPKSPTKGGC

SEQ ID NO: 1069 - protein, 30 aa, Synthetic construct; identical to SEQ ID NO: 238

SEQ ID NO: 1070 - protein, 30 aa, Synthetic construct
AIPVKRGATGETKVFIENSNSPKSPTKGGC

SEQ ID NO: 1071 - protein, 30 aa, Synthetic construct
AIPVKRGATGETKVFIGESNSPKSPTKGGC

SEQ ID NO: 1072 - protein, 30 aa, Synthetic construct
AIPVKRGATGETKVFIGNENSPKSPTKGGC

SEQ ID NO: 1073 - protein, 30 aa, Synthetic construct
AIPVKRGATGETKVFIGNSESPKSPTKGGC

SEQ ID NO: 1074 - protein, 30 aa, Synthetic construct
AIPVKRGATGETKVFIGNSNEPKSPTKGGC

SEQ ID NO: 1075 - protein, 30 aa, Synthetic construct
AIPVKRGATGETKVFIGNSNSEKSPTKGGC

SEQ ID NO: 1076 - protein, 30 aa, Synthetic construct
AIPVKRGATGETKVFIGNSNSPESPTKGGC

SEQ ID NO: 1077 - protein, 30 aa, Synthetic construct
AIPVKRGATGETKVFIGNSNSPKEPTKGGC

SEQ ID NO: 1078 - protein, 30 aa, Synthetic construct
AIPVKRGATGETKVFIGNSNSPKSETKGGC

SEQ ID NO: 1079 - protein, 30 aa, Synthetic construct
AIPVKRGATGETKVFIGNSNSPKSPEKGGC

SEQ ID NO: 1080 - protein, 30 aa, Synthetic construct
AIPVKRGATGETKVFIGNSNSPKSPTEGGC

SEQ ID NO: 1081 - protein, 30 aa, Synthetic construct
AIPVKRGATGETKVFIGNSNSPKSPTKEGC

SEQ ID NO: 1082 - protein, 30 aa, Synthetic construct
AIPVKRGATGETKVFIGNSNSPKSPTKGEC

SEQ ID NO: 1083 - protein, 30 aa, Synthetic construct
AIPVKRGATGETKVFIGNSNSPKSPTKGGE

SEQ ID NO: 1084 - protein, 6 aa, Synthetic construct
GGSGGS

SEQ ID NO: 1085 - protein, 20 aa, Synthetic construct
GSGGGSGGSGGGGSGGSGGS

SEQ ID NO: 1086 - protein, 108 aa, Synthetic construct
GSYALGPYQISAPQLPAYNGQTVGTFYYVNDAGGLESKVFSSGGPTPYPNYANAGHVAGQSALFMRDNGISEGLVFHNNPEGTCGFCVNMIETLLPENAKMTVVPPKG

SEQ ID NO: 1087 - protein, 13 aa, Synthetic construct
YKCPECGKSFSQK